(19) United States
(12) Patent Application Publication
(10) Pub. No.: US 2023/0225196 A1
(43) Pub. Date: Jul. 13, 2023

(54) MATERIALS FOR ELECTRONIC DEVICES

(71) Applicant: Merck Patent GmbH, Darmstadt (DE)

(72) Inventors: Jens ENGELHART, Darmstadt (DE); Elvira MONTENEGRO, Darmstadt (DE); Simon SIEMIANOWSKI, Darmstadt (DE)

(21) Appl. No.: 17/927,376

(22) PCT Filed: May 25, 2021
(86) PCT No.: PCT/EP2021/063873

§ 371 Date: Nov. 23, 2022

(30) Foreign Application Priority Data
May 27, 2020 (EP) 20176926.2

(51) Int. Cl. H10K 85/60; C07D 307/91; C09K 11/06; C07D 333/76

(52) U.S. Cl. CPC H10K 85/633; C07D 307/91; C09K 11/06; H10K 85/636; C07D 333/76; H10K 50/181

(57) ABSTRACT

The present application relates to materials for use in electronic devices, to processes for preparing the materials, and to electronic devices containing the materials.

The present application relates to aromatic and heteroaromatic compounds, to the preparation thereof, to mixtures and formulations comprising the compounds and to electronic devices comprising the compounds or the mixtures.

Electronic devices in the context of this application are understood to mean what are called organic electronic devices, which comprise organic semiconductor materials as functional materials. More particularly, these are understood to mean organic electroluminescent devices, and OLEDs (organic light-emitting diodes) are very particularly preferred organic electroluminescent devices. The term OLEDs is understood to mean organic electroluminescent devices which have one or more layers comprising organic compounds and emit light on application of electrical voltage. The construction and general principle of function of OLEDs are known to those skilled in the art.

In electronic devices, especially OLEDs, there is great interest in an improvement in the performance data. In these aspects, it has not yet been possible to find any entirely satisfactory solution.

A great influence on the performance data of electronic devices is possessed by emission layers and layers having a hole-transporting function. These include not only the emission layer but especially also the hole transport layer (HTL), the electron blocker layer (EBL) and the hole injection layer (HIL). There is a continuing search for novel compounds for use in these layers, especially hole-transporting compounds and compounds that can serve as hole-conducting electron blocker material in an electron blocker layer, as hole conductor in a hole transport layer or as hole-transporting matrix material, especially for phosphorescent emitters, in an emitting layer. For this purpose, there is a search especially for compounds that have a high glass transition temperature, high stability, and high conductivity for holes. A high stability of the compound is a prerequisite for achieving a long lifetime of the electronic device.

In the prior art, triarylamine compounds in particular are known as electron blocker and hole transport materials, and hole-transporting matrix materials for electronic devices. The triarylamine compounds known for use in electronic devices also include fluorenylamine compounds, i.e. triarylamine compounds in which at least one aryl group is a fluorenyl group.

However, there is still a need for alternative compounds suitable for use in electronic devices, especially for compounds having one or more of the abovementioned advantageous properties. There is still a need for improvement in the performance data achieved when the compounds are used in electronic devices, especially in respect of lifetime, operating voltage and also efficiency of the devices.

It has been found that particular fluorenylamine compounds specified in detail further down that contain particular linkers L between a fluorenyl group and an amine group, and that are substituted by aromatic and heteroaromatic groups in a specific manner on the amine, are of excellent suitability for use in organic electronic devices, especially for use in OLEDs, more particularly for use as hole transport materials, for use as hole-transporting matrix materials, especially for phosphorescent emitters, and very particularly for use as electron block materials (EBM) in an electron blocker layer (EBL). Performance data of the devices containing the compounds are distinctly improved over the prior art. More particularly, the lifetimes and operating voltages of the devices have distinctly improved values.

The compounds themselves also have high stability, especially with respect to air and light. The compounds are notable for high storage stability. In addition, the compounds have a high glass transition temperature and high conductivity for holes.

The inventive compounds relate to those of the following formula (1):

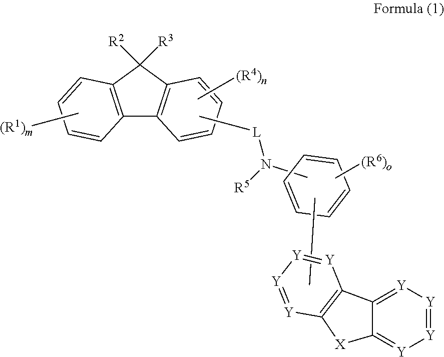

where the variables that occur are as follows:
X is either O or S, where X in a very particularly preferred embodiment is O; in another preferred embodiment, X is S;
Y is the same or different at each instance and is CR⁷ or N, it being preferable when Y is CR⁷;
L is a divalent group that connects the fluorenyl group to the amine group in formula (1), where L is a divalent aromatic ring system having 6 to 40 aromatic ring atoms, L preferably being a divalent group selected from the following formulae (L-1), (L-2), (L-3), (L-4) and (L-5):

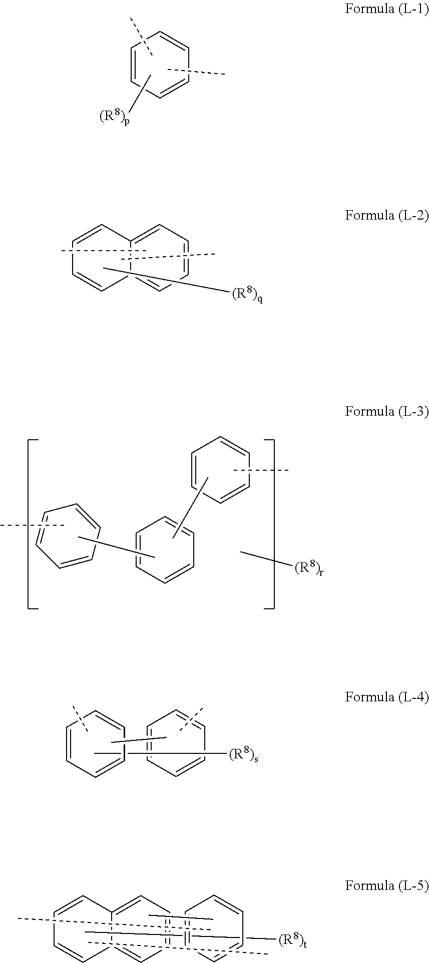

where one of the two dotted lines indicates the bond of the L group to the nitrogen on the one hand and the other dotted line the bond to the fluorenyl group on the other hand, and where the groups of the formulae (L-1), (L-2), (L-3), (L-4) and (L-5) are a phenylene, naphthylene, terphenylene, biphenylene or naphthyl-phenyl group which is substituted by one or more R⁸ radicals or unsubstituted.

The notation for the R⁹ group in formula (L-2) and in formula (L-3) means that the R⁸ radical(s) may occur in both rings of the formula (L-2) or in all three rings of the formula (L-3).

R¹, R⁴, R⁶, R⁷ and R⁸ are the same or different at each instance and are selected from H, D, F, Cl, Br, I, C(═O)R¹¹, CN, Si(R¹¹)₃, N(R¹¹)₂, P(═O)(R¹¹)₂, OR¹¹, S(═O)R¹¹, S(═O)₂R¹¹, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ radicals may be joined to one another and may form a ring and/or two or more R⁴ radicals may be joined to one another and may form a ring and/or two or more R⁶ radicals may be joined to one another and may form a ring and/or two or more R⁷ radicals may be joined to one another and may form a ring and/or two or more R⁸ radicals may be joined to one another and may form a ring; where the alkyl, alkoxy, alkenyl and alkynyl groups mentioned and the aromatic ring systems and heteroaromatic ring systems mentioned may each be substituted by R¹¹ radicals; and where one or more CH₂ groups in the alkyl, alkoxy, alkenyl and alkynyl groups may be replaced by —R¹¹C═CR¹¹—, —C≡C—, Si(R¹¹)₂, C═O, C═NR¹¹, —C(═O)O—, —C(═O)NR¹¹—, NR¹¹, P(═O)(R¹¹), —O—, —S—, SO or SO₂; preferably, two or more R¹ radicals do not form a ring with one another and/or two or more R⁴ radicals do not form a ring with one another, and/or two or more R⁶ radicals do not form a ring with one another and/or two or more R⁷ radicals do not form a ring with one another and/or two or more R⁸ radicals do not form a ring with one another;

R² and R³ are the same or different at each instance and are selected from straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the two R² and R³ radicals may be joined to one another and may form a ring; where the alkyl, alkoxy, alkenyl and alkynyl groups mentioned and the aromatic ring systems and heteroaromatic ring systems mentioned may each be substituted by R¹¹ radicals; and where one or more CH₂ groups in the alkyl, alkoxy, alkenyl and alkynyl groups may be replaced by —R¹¹C═CR¹¹—, —C≡C—, Si(R¹¹)₂, C═O, C═NR¹¹, —C(═O)O—, —C(═O)NR¹¹—, NR¹¹, P(═O)(R¹¹), —O—, —S—, SO or SO₂; if the two R² and R³ radicals form a ring, the result is a spiro compound, preferably a spirobifluorene; it is particularly preferable when the two R² and R³ radicals do not form a ring with one another;
R⁵ is an aromatic ring system which has 6 to 40 aromatic ring atoms and may be substituted by one or more R¹¹ radicals, or a heteroaromatic ring system which has 5 to 40 aromatic ring atoms and may be substituted by one or more R¹¹ radicals;
R¹¹ is the same or different at each instance and is selected from H, D, F, Cl, Br, I, C(═O)R¹², CN, Si(R¹²)₃, N(R¹²)₂, P(═O)(R¹²)₂, OR¹², S(═O)R¹², S(═O)₂R¹², straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹¹ radicals may be joined to one another and may form a ring; where the alkyl, alkoxy, alkenyl and alkynyl groups mentioned and the aromatic ring systems and heteroaromatic ring systems mentioned are each substituted by R¹² radicals; and where one or more CH₂ groups in the alkyl, alkoxy, alkenyl and alkynyl groups mentioned may be replaced by —R¹²C═CR¹²—, —C≡C—, Si(R¹²)₂, C═O, C═NR¹², —C(═O)O—, —C(═O)NR¹²—, NR¹², P(═O)(R¹²), —O—, —S—, SO or SO₂; where two or more R¹¹ radicals may not form a ring with one another;
R¹² is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where the alkyl, alkoxy, alkenyl and alkynyl groups, aromatic ring systems and heteroaromatic ring systems mentioned may be substituted by one or more radicals selected from F and CN;
m is 0, 1, 2, 3 or 4; it is preferable when m is 0 or 1, very preferable when m is 1 and most preferable when m is 0;
n is 0, 1, 2 or 3; it is preferable when n is 0 or 1, very preferable when n is 1 and most preferable when n is 0;
o is 0, 1, 2 or 3; it is preferable when o is 0 or 1, very preferable when o is 1 and most preferable when o is 0;
p is 0, 1, 2, 3 or 4; it is preferable when p is 0 or 1, very preferable when p is 1 and most preferable when p is 0;
q is 0, 1, 2, 3, 4, 5 or 6; it is preferable when q is 0 or 1, very preferable when q is 1 and most preferable when q is 0;
r is 0, 1, 2, 3, 4, 5, 7, 8, 9, 10, 11 or 12; it is preferable when r is 0 or 1, very preferable when r is 1 and most preferable when r is 0;
s is 0, 1, 2, 3, 4, 5, 7 or 8; it is preferable when s is 0 or 1, very preferable when s is 1 and most preferable when s is 0;
t is 0, 1, 2, 3, 4, 5, 7, 8, 9 or 10; it is preferable when t is 0 or 1, very preferable when t is 1 and most preferable when t is 0.

In a preferred embodiment, the R¹ radical does not contain any fused aromatic and/or heteroaromatic ring systems having more than 12 aromatic carbon atoms as ring atoms in the aromatic or heteroaromatic ring system; very preferably, the R¹ radical does not contain any fused aromatic and/or heteroaromatic ring systems having more than 10 aromatic carbon atoms as ring atoms in the aromatic or heteroaromatic ring system; even more preferably, the R¹ radical does not contain any carbazoles; especially preferably, the R¹ radical does not contain any fused heteroaromatic ring systems; and most preferably, the R¹ radical contains neither aromatic nor heteroaromatic fused ring systems.

In a preferred embodiment, the R² radical does not contain any fused aromatic and/or heteroaromatic ring systems having more than 12 aromatic carbon atoms as ring atoms in the aromatic or heteroaromatic ring system; very preferably, the R² radical does not contain any fused aromatic and/or heteroaromatic ring systems having more than 10 aromatic carbon atoms as ring atoms in the aromatic or heteroaromatic ring system; even more preferably, the R² radical does not contain any carbazoles; especially preferably, the R² radical does not contain any fused heteroaromatic ring systems; and most preferably, the R² radical contains neither aromatic nor heteroaromatic fused ring systems.

In a further preferred embodiment, the R³ radical does not contain any fused aromatic and/or heteroaromatic ring systems having more than 12 aromatic carbon atoms as ring atoms in the aromatic or heteroaromatic ring system; very preferably, the R³ radical does not contain any fused aromatic and/or heteroaromatic ring systems having more than 10 aromatic carbon atoms as ring atoms in the aromatic or heteroaromatic ring system; even more preferably, the R³ radical does not contain any carbazoles; especially preferably, the R³ radical does not contain any fused heteroaromatic ring systems; and most preferably, the R³ radical contains neither aromatic nor heteroaromatic fused ring systems.

In yet a further preferred embodiment, the R⁴ radical does not contain any fused aromatic and/or heteroaromatic ring systems having more than 12 aromatic carbon atoms as ring atoms in the aromatic or heteroaromatic ring system; very preferably, the R⁴ radical does not contain any fused aromatic and/or heteroaromatic ring systems having more than 10 aromatic carbon atoms as ring atoms in the aromatic or heteroaromatic ring system; even more preferably, the R⁴ radical does not contain any carbazoles; especially preferably, the R⁴ radical does not contain any fused heteroaromatic ring systems; and most preferably, the R⁴ radical contains neither aromatic nor heteroaromatic fused ring systems.

In yet a further preferred embodiment, the R⁵ radical does not contain any fused aromatic and/or heteroaromatic ring systems having more than 12 aromatic carbon atoms as ring atoms in the aromatic or heteroaromatic ring system; very preferably, the R⁵ radical does not contain any fused aromatic and/or heteroaromatic ring systems having more than 10 aromatic carbon atoms as ring atoms in the aromatic or heteroaromatic ring system; even more preferably, the R⁵ radical does not contain any carbazoles; especially preferably, the R⁵ radical does not contain any fused heteroaromatic ring systems; and most preferably, the R⁵ radical contains neither aromatic nor heteroaromatic fused ring systems.

In yet a further preferred embodiment, the R⁶ radical does not contain any fused aromatic and/or heteroaromatic ring systems having more than 12 aromatic carbon atoms as ring atoms in the aromatic or heteroaromatic ring system; very preferably, the R⁶ radical does not contain any fused aromatic and/or heteroaromatic ring systems having more than 10 aromatic carbon atoms as ring atoms in the aromatic or heteroaromatic ring system; even more preferably, the R⁶ radical does not contain any carbazoles; especially preferably, the R⁶ radical does not contain any fused heteroaromatic ring systems; and most preferably, the R⁶ radical contains neither aromatic nor heteroaromatic fused ring systems.

In yet a further preferred embodiment, the R⁷ radical does not contain any fused aromatic and/or heteroaromatic ring systems having more than 12 aromatic carbon atoms as ring atoms in the aromatic or heteroaromatic ring system; very preferably, the R⁷ radical does not contain any fused aromatic and/or heteroaromatic ring systems having more than 10 aromatic carbon atoms as ring atoms in the aromatic or heteroaromatic ring system; even more preferably, the R⁷ radical does not contain any carbazoles; especially preferably, the R⁷ radical does not contain any fused heteroaromatic ring systems; and most preferably, the R⁷ radical contains neither aromatic nor heteroaromatic fused ring systems.

In yet a further preferred embodiment, the R⁸ radical does not contain any fused aromatic and/or heteroaromatic ring systems having more than 12 aromatic carbon atoms as ring atoms in the aromatic or heteroaromatic ring system; very preferably, the R⁸ radical does not contain any fused aromatic and/or heteroaromatic ring systems having more than 10 aromatic carbon atoms as ring atoms in the aromatic or heteroaromatic ring system; even more preferably, the R⁸ radical does not contain any carbazoles; especially preferably, the R⁸ radical does not contain any fused heteroaromatic ring systems; and most preferably, the R⁸ radical contains neither aromatic nor heteroaromatic fused ring systems.

In yet a further preferred embodiment, the R¹¹ radical does not contain any fused aromatic and/or heteroaromatic ring systems having more than 12 aromatic carbon atoms as ring atoms in the aromatic or heteroaromatic ring system; very preferably, the R¹¹ radical does not contain any fused aromatic and/or heteroaromatic ring systems having more than 10 aromatic carbon atoms as ring atoms in the aromatic or heteroaromatic ring system; even more preferably, the R¹¹ radical does not contain any carbazoles; especially preferably, the R¹¹ radical does not contain any fused heteroaromatic ring systems; and most preferably, the R¹¹ radical contains neither aromatic nor heteroaromatic fused ring systems.

In yet a further preferred embodiment, the R¹² radical does not contain any fused aromatic and/or heteroaromatic ring systems having more than 12 aromatic carbon atoms as ring atoms in the aromatic or heteroaromatic ring system; very preferably, the R¹² radical does not contain any fused aromatic and/or heteroaromatic ring systems having more than 10 aromatic carbon atoms as ring atoms in the aromatic or heteroaromatic ring system; even more preferably, the R¹² radical does not contain any carbazoles; especially preferably, the R¹² radical does not contain any fused heteroaromatic ring systems; and most preferably, the R¹² radical contains neither aromatic nor heteroaromatic fused ring systems.

In a particularly preferred embodiment, none of the R¹ to R⁸ radicals contains fused aromatic and/or heteroaromatic ring systems having more than 12 aromatic carbon atoms as ring atoms in the aromatic or heteroaromatic ring system; very preferably, none of the R¹ to R⁸ radicals contains fused aromatic and/or heteroaromatic ring systems having more than 10 aromatic carbon atoms as ring atoms in the aromatic or heteroaromatic ring system; even more preferably, none of the R¹ to R⁸ radicals contains carbazoles; especially preferably, none of the R¹ to R⁸ radicals contains fused heteroaromatic ring systems; and most preferably, none of the R¹ to R⁸ radicals contains aromatic nor heteroaromatic fused ring systems.

In a particularly preferred embodiment, none of the R¹ to R⁸, R¹¹ and R¹² radicals contains fused aromatic and/or heteroaromatic ring systems having more than 12 aromatic carbon atoms as ring atoms in the aromatic or heteroaromatic ring system; very preferably, none of the R¹ to R⁸, R¹¹ and R¹² radicals contains fused aromatic and/or heteroaromatic ring systems having more than 10 aromatic carbon atoms as ring atoms in the aromatic or heteroaromatic ring system; even more preferably, none of the R¹ to R⁸, R¹¹ and R¹² radicals contains carbazoles; especially preferably, none of the R¹ to R⁸, R¹¹ and R¹² radicals contains fused heteroaromatic ring systems; and most preferably, none of the R¹ to R⁸, R¹¹ and R¹² radicals contains aromatic nor heteroaromatic fused ring systems.

The definitions which follow are applicable to the chemical groups that are used in the present applications. They are applicable unless any more specific definitions are given.

An aryl group in the context of this invention is understood to mean either a single aromatic cycle, i.e. benzene, or a fused aromatic polycycle, for example naphthalene, phenanthrene or anthracene. A fused aromatic polycycle in the context of the present application consists of two or more single aromatic cycles fused to one another. Fusion between cycles is understood here to mean that the cycles share at least one edge with one another. An aryl group in the context of this invention contains 6 to 40 aromatic ring atoms of which none is a heteroatom.

A heteroaryl group in the context of this invention is understood to mean either a single heteroaromatic cycle, for example pyridine, pyrimidine or thiophene, or a fused heteroaromatic polycycle, for example quinoline or carbazole. A fused heteroaromatic polycycle in the context of the present application consists of two or more single aromatic or heteroaromatic cycles that are fused to one another, where at least one of the aromatic and heteroaromatic cycles is a heteroaromatic cycle. Fusion between cycles is understood here to mean that the cycles share at least one edge with one another. A heteroaryl group in the context of this invention contains 5 to 40 aromatic ring atoms of which at least one is a heteroatom. The heteroatoms of the heteroaryl group are preferably selected from N, O and S.

An aryl or heteroaryl group, each of which may be substituted by the abovementioned radicals, is especially understood to mean groups derived from benzene, naphthalene, anthracene, phenanthrene, pyrene, dihydropyrene, chrysene, perylene, triphenylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, benzimidazolo[1,2-a]benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-tiazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the context of this invention is a system which does not necessarily contain solely aryl groups, but which may additionally contain one or more nonaromatic rings fused to at least one aryl group. These nonaromatic rings contain exclusively carbon atoms as ring atoms. Examples of groups covered by this definition are tetrahydronaphthalene, fluorene and spirobifluorene. In addition, the term “aromatic ring system” includes systems that consist of two or more aromatic ring systems joined to one another via single bonds, for example biphenyl, terphenyl, 7-phenyl-2-fluorenyl, quaterphenyl and 3,5-diphenyl-1-phenyl. An aromatic ring system in the context of this invention contains 6 to 40 carbon atoms and no heteroatoms in the ring system. The definition of “aromatic ring system” does not include heteroaryl groups.

A heteroaromatic ring system conforms to the abovementioned definition of an aromatic ring system, except that it must contain at least one heteroatom as ring atom. As is the case for the aromatic ring system, the heteroaromatic ring system need not contain exclusively aryl groups and heteroaryl groups, but may additionally contain one or more nonaromatic rings fused to at least one aryl or heteroaryl group. The nonaromatic rings may contain exclusively carbon atoms as ring atoms, or they may additionally contain one or more heteroatoms, where the heteroatoms are preferably selected from N, O and S. One example of such a heteroaromatic ring system is benzopyranyl. In addition, the term “heteroaromatic ring system” is understood to mean systems that consist of two or more aromatic or heteroaromatic ring systems that are bonded to one another via single bonds, for example 4,6-diphenyl-2-triazinyl. A heteroaromatic ring system in the context of this invention contains 5 to 40 ring atoms selected from carbon and heteroatoms, where at least one of the ring atoms is a heteroatom. The heteroatoms of the heteroaromatic ring system are preferably selected from N, O and S.

The terms “heteroaromatic ring system” and “aromatic ring system” as defined in the present application thus differ from one another in that an aromatic ring system cannot have a heteroatom as ring atom, whereas a heteroaromatic ring system must have at least one heteroatom as ring atom. This heteroatom may be present as a ring atom of a nonaromatic heterocyclic ring or as a ring atom of an aromatic heterocyclic ring.

In accordance with the above definitions, any aryl group is covered by the term “aromatic ring system”, and any heteroaryl group is covered by the term “heteroaromatic ring system”.

An aromatic ring system having 6 to 40 aromatic ring atoms or a heteroaromatic ring system having 5 to 40 aromatic ring atoms is especially understood to mean groups derived from the groups mentioned above under aryl groups and heteroaryl groups, and from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, indenocarbazole, or from combinations of these groups.

In the context of the present invention, a straight-chain alkyl group having 1 to 20 carbon atoms and a branched or cyclic alkyl group having 3 to 20 carbon atoms and an alkenyl or alkynyl group having 2 to 40 carbon atoms in which individual hydrogen atoms or CH₂ groups may also be substituted by the groups mentioned above in the definition of the radicals are preferably understood to mean the methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals.

An alkoxy or thioalkyl group having 1 to 20 carbon atoms in which individual hydrogen atoms or CH₂ groups may also be replaced by the groups mentioned above in the definition of the radicals is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The wording that two or more radicals together may form a ring, in the context of the present application, shall be understood to mean, inter alia, that the two radicals are joined to one another by a chemical bond. In addition, however, the abovementioned wording shall also be understood to mean that, if one of the two radicals is hydrogen, the second radical binds to the position to which the hydrogen atom was bonded, forming a ring. The compound of the formula (1) is preferably a monoamine. A monoamine is understood to mean a compound containing a single triarylamino group and no further triarylamino groups, more preferably a compound containing a single amino group and no further amino groups.

The compound of the invention preferably has the general formula (2)

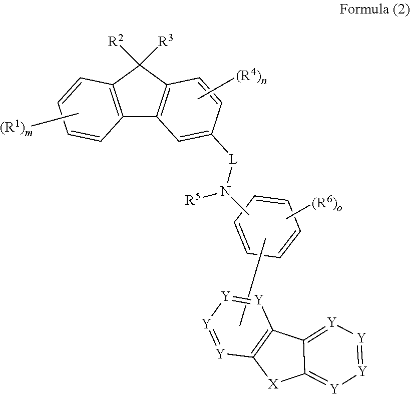

In a preferred embodiment, the L group is a group of the formula (L-1). In another preferred embodiment, the L group is a group of the formula (L-2).

And in yet another preferred embodiment, the L group is a group of the formula (L-3), where the following groups are very preferred among the groups of the formula (L-3):

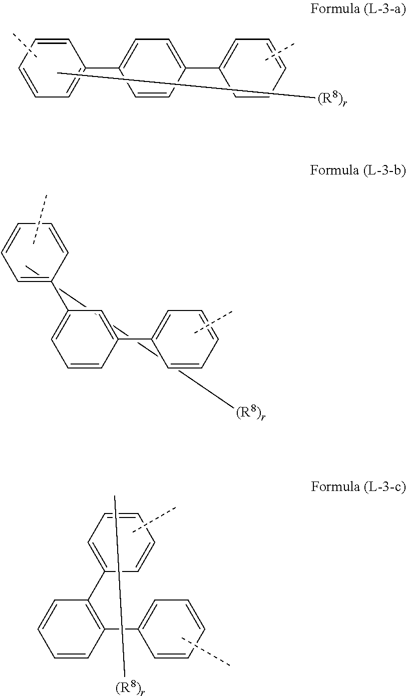

Entirely analogously to the above description of the formula (L-3), the notation in the formulae (L-3-a) to (L-3-c) means that the R⁸ radicals may occur in all three aromatic rings.

In another preferred embodiment, the L group is a group of the formula (L-4).

In another preferred embodiment, the L group is a group of the formula (L-5).

L is most preferably selected from the following groups:

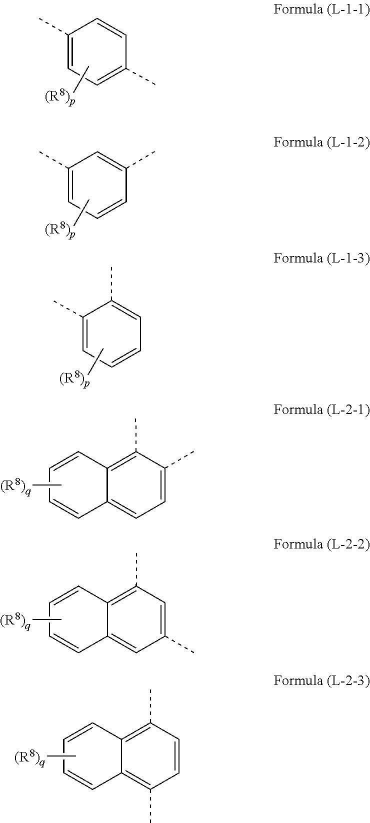

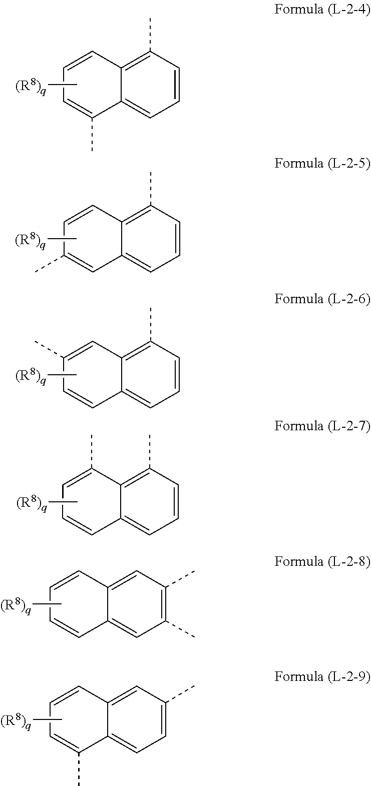

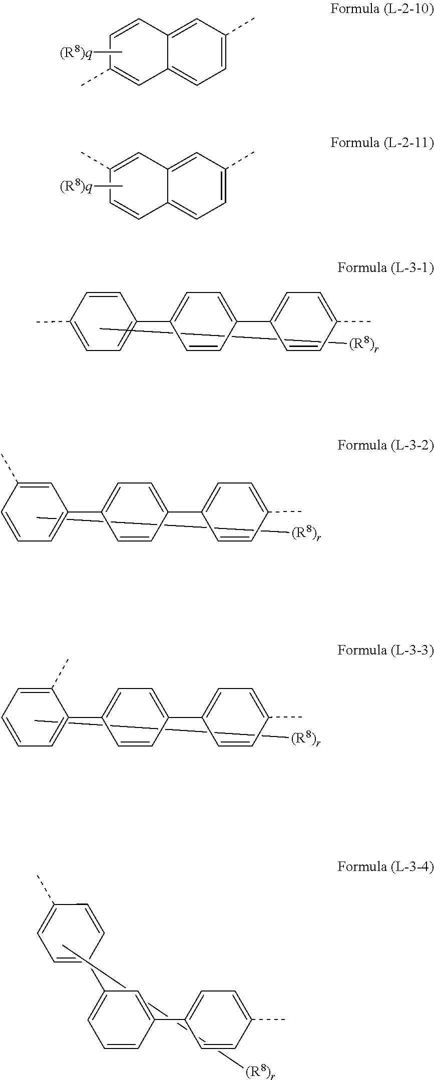

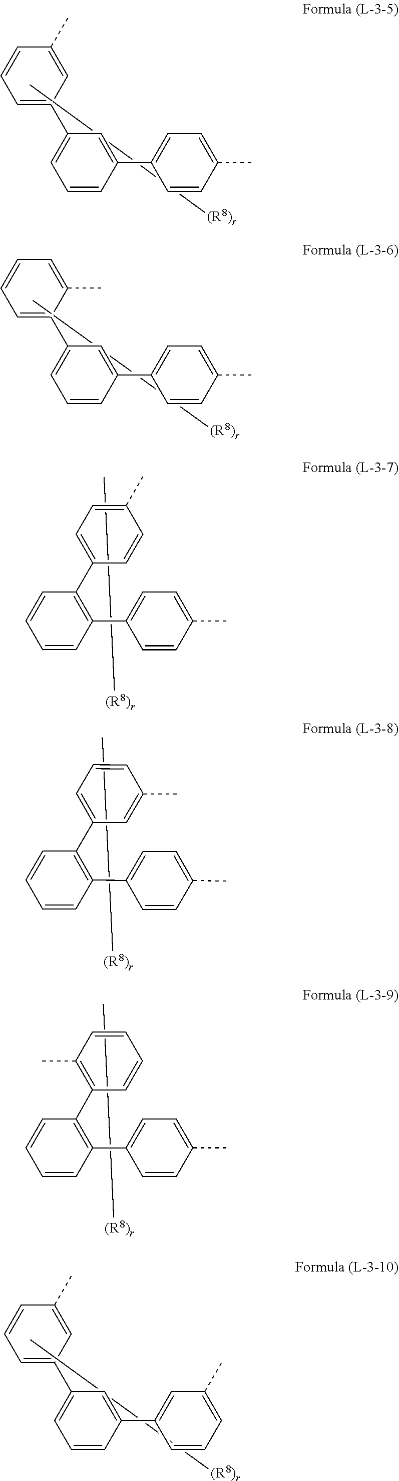

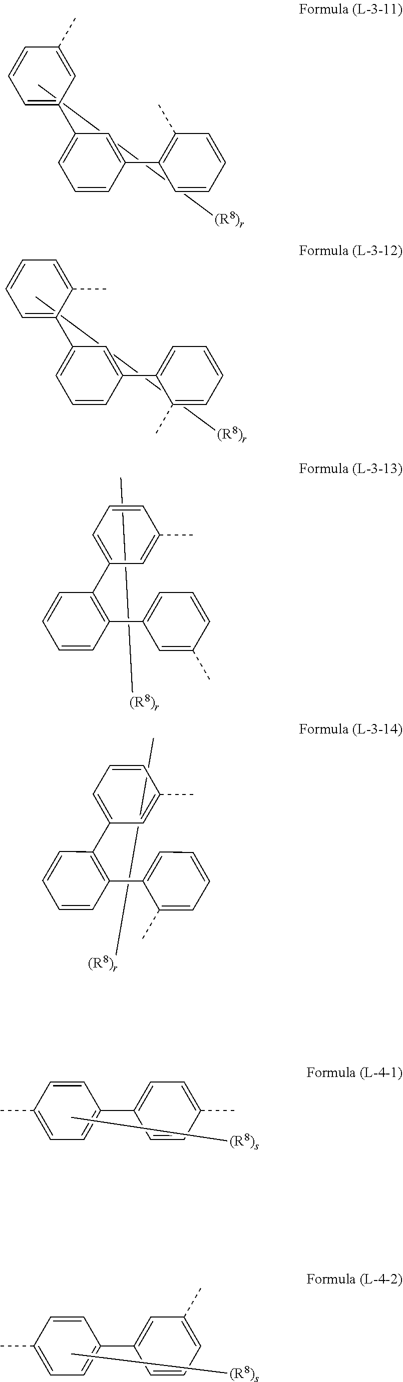

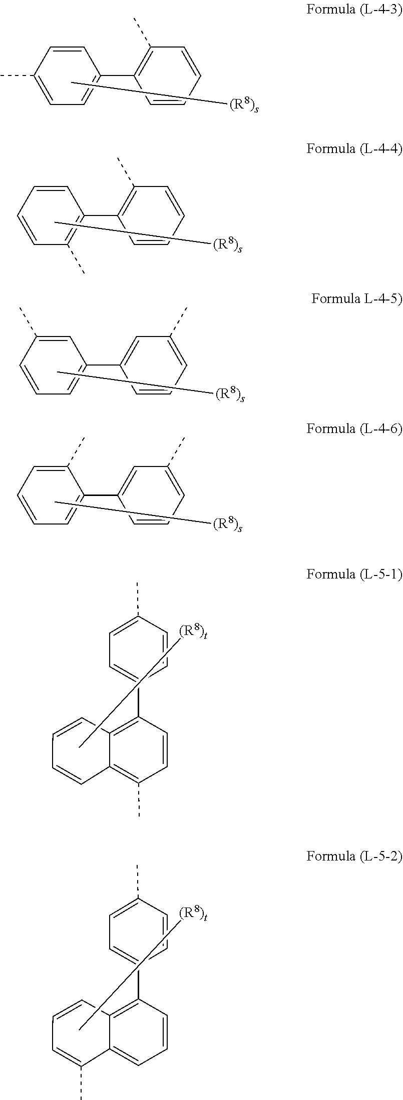

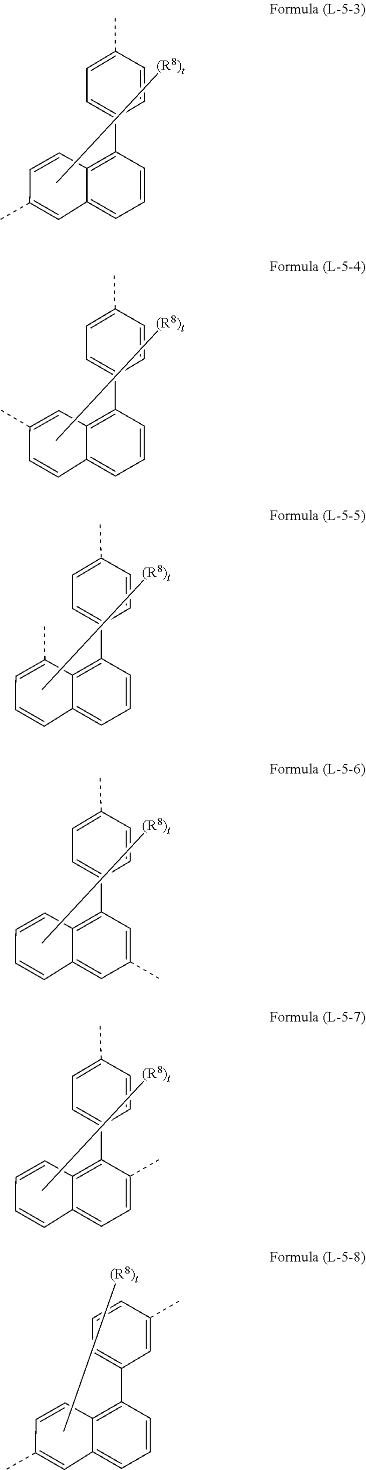

Among these, especially preferred L groups are those of the formulae (L-1-1) to (L-1-3).

Further especially preferred L groups are those of the formulae (L-2-1) to (L-2-11).

Further especially preferred L groups are those of the formulae (L-3-1) to (L-3-14).

Further especially preferred L groups are those of the formulae (L-4-1) to (L-4-6).

Further especially preferred L groups are those of the formulae (L-5-1) to (L-5-8).

Further preferred compounds of the invention are selected from the following formulae (3) to (6):

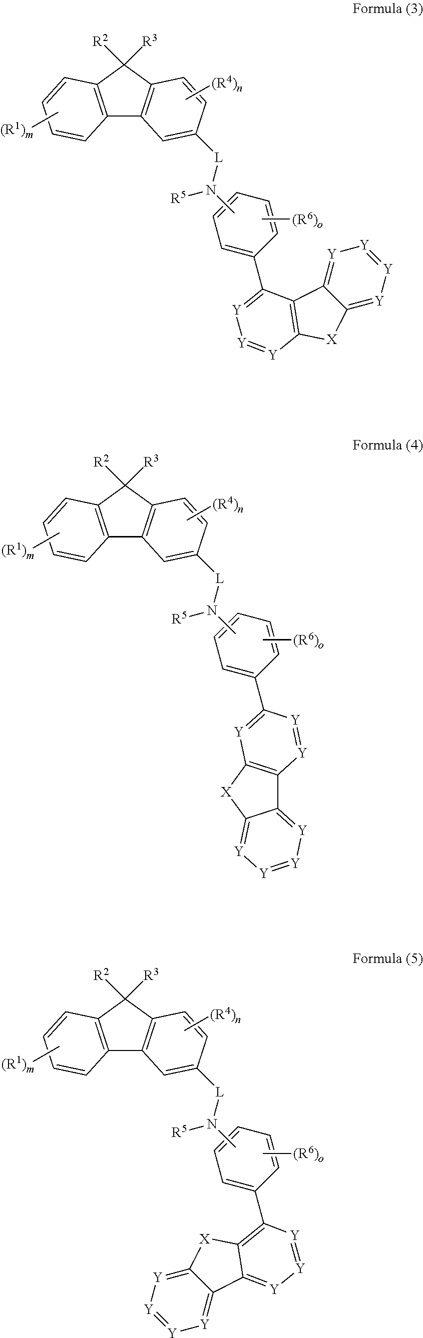

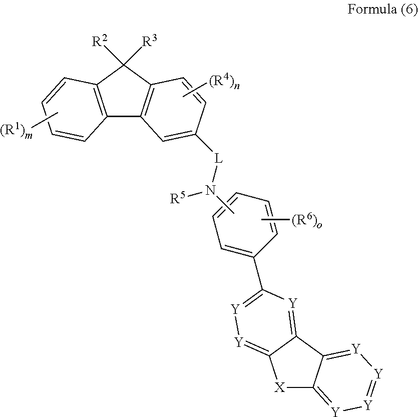

Even more preferred compounds of the invention are selected from the following formulae (7) to (10):

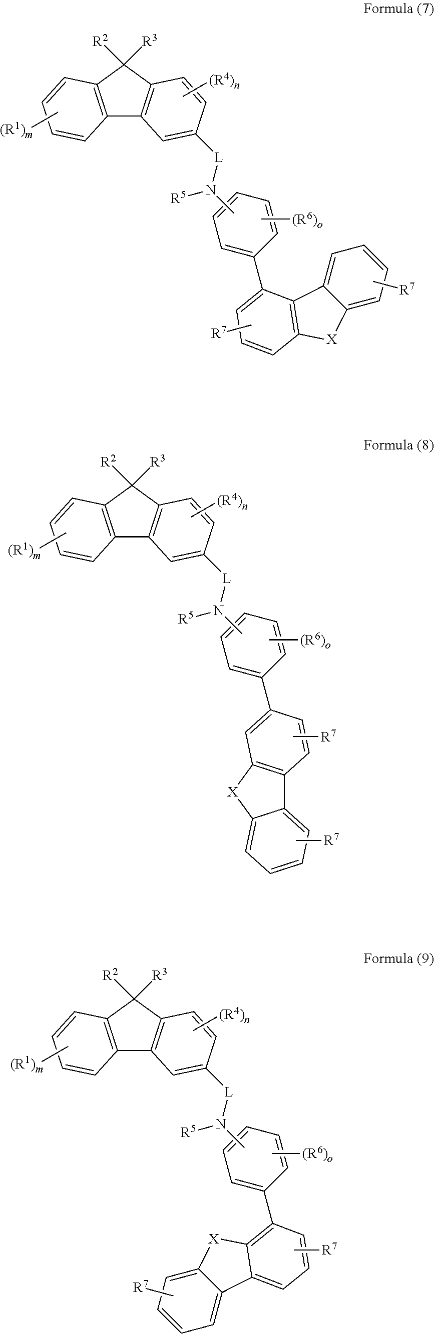

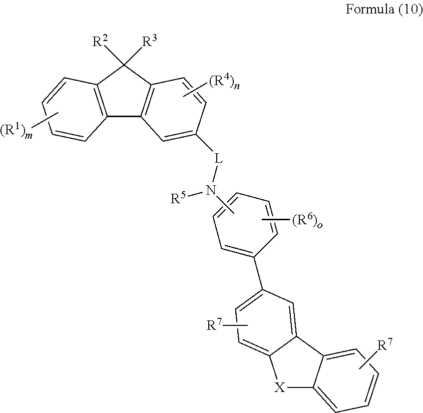

Very particularly preferred compounds of the invention are those of the formulae (11) to (22).

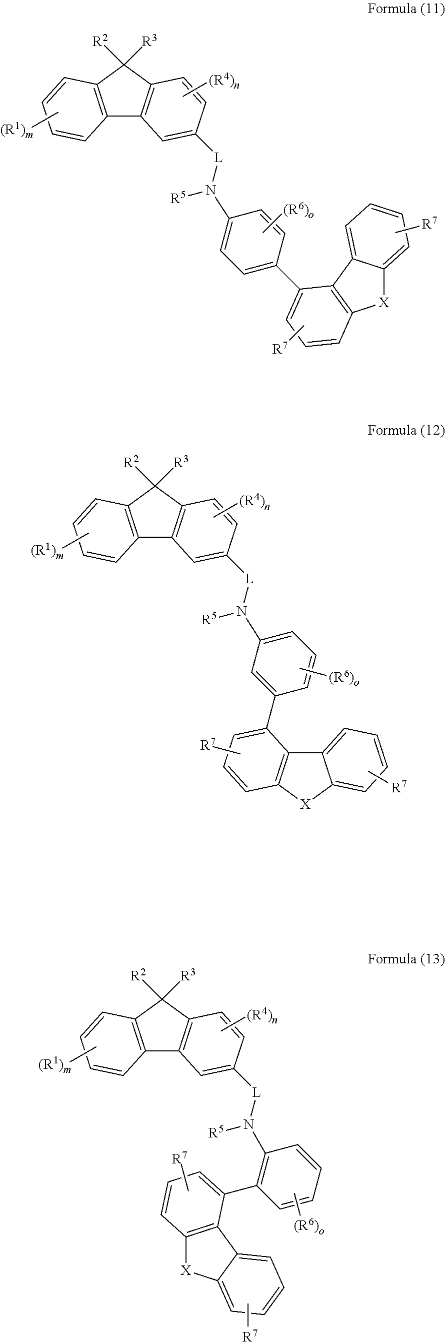

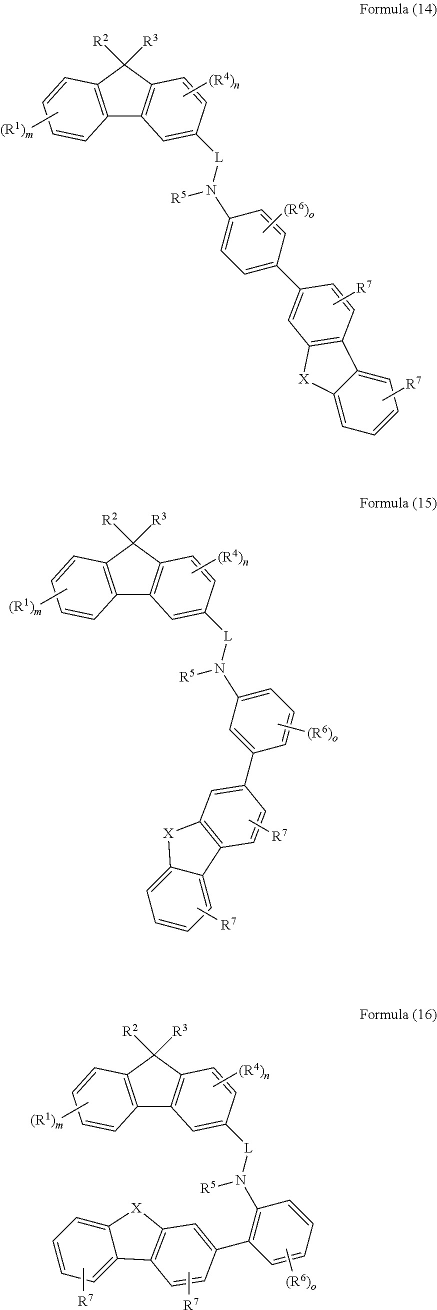

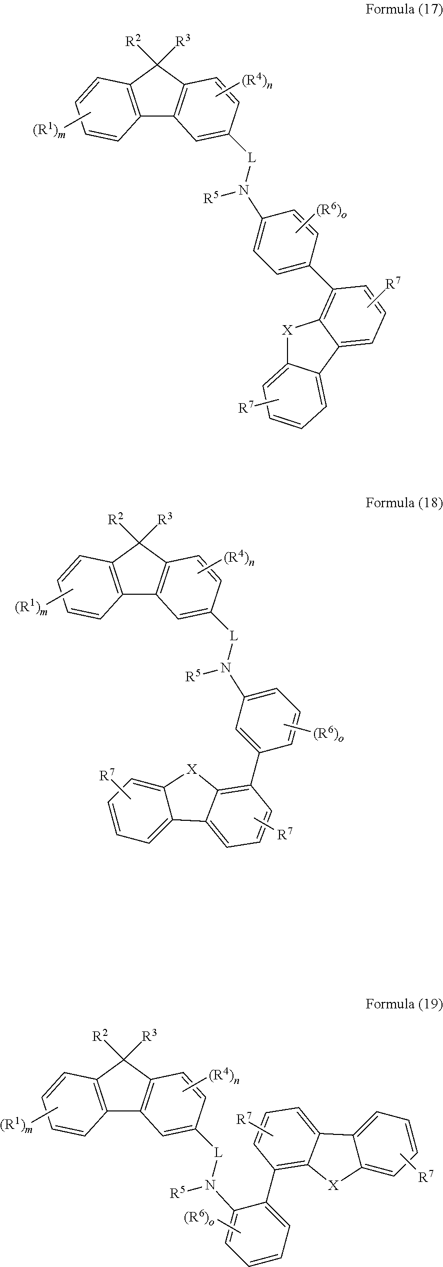

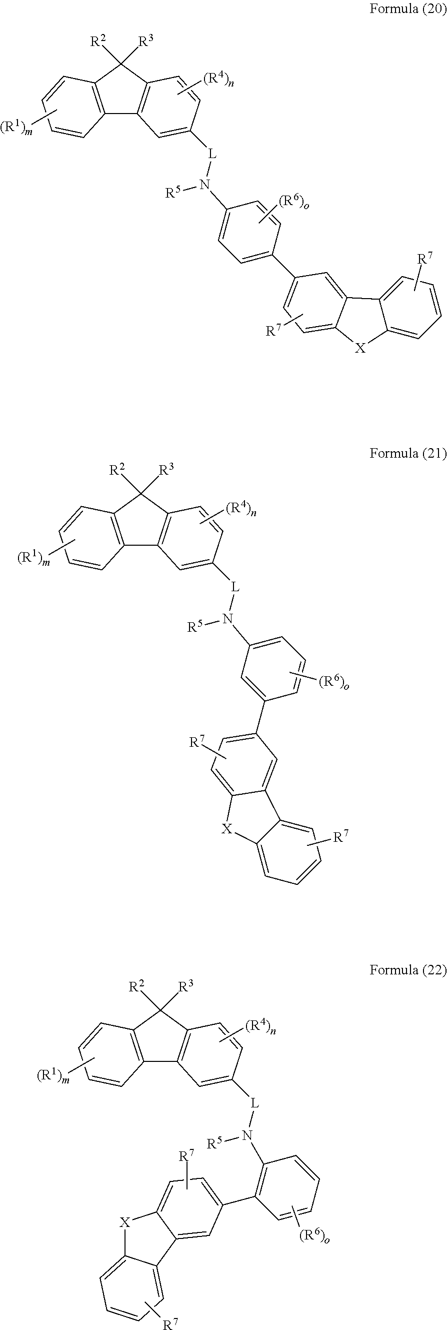

Very particularly preferred compounds of the invention are those of the formulae (23) to (34).

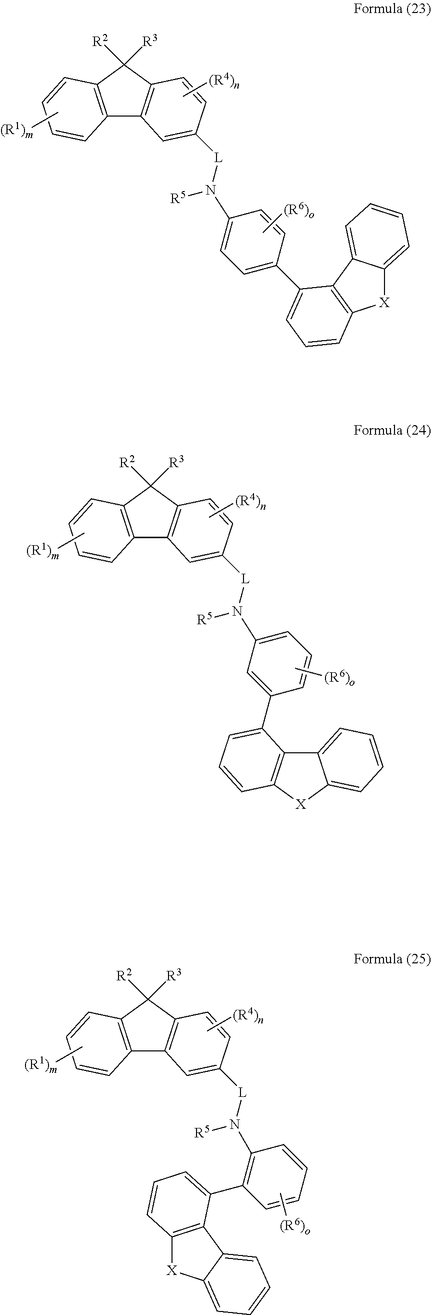

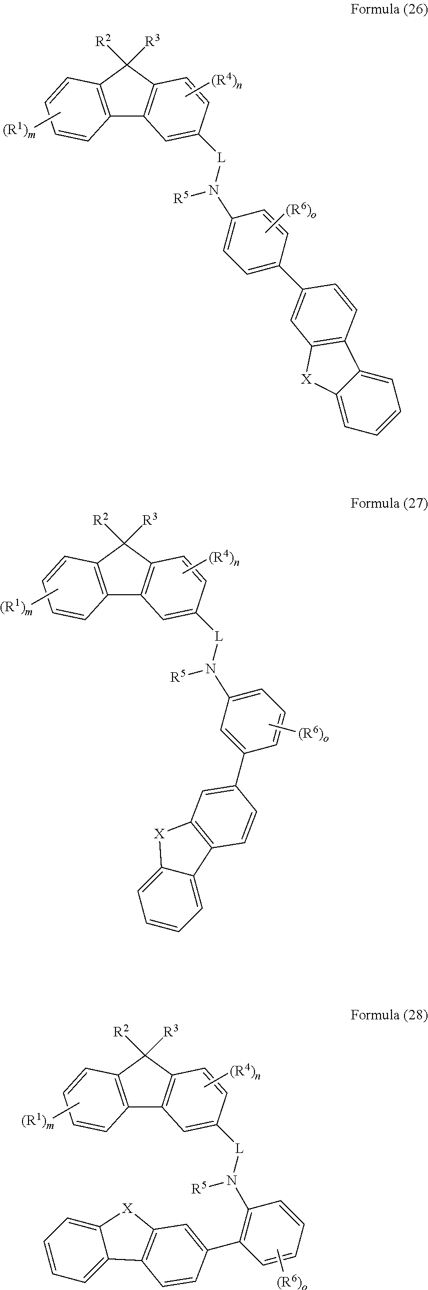

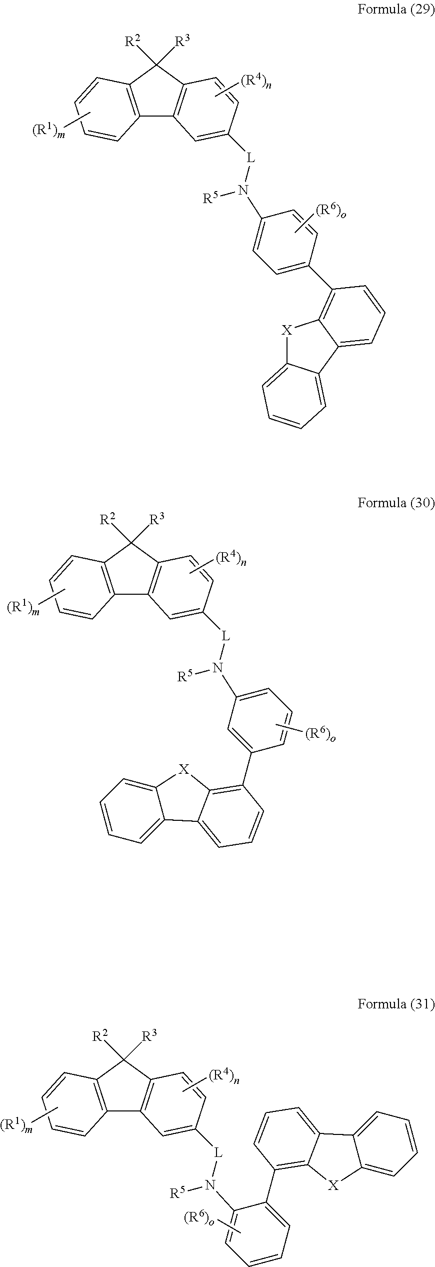

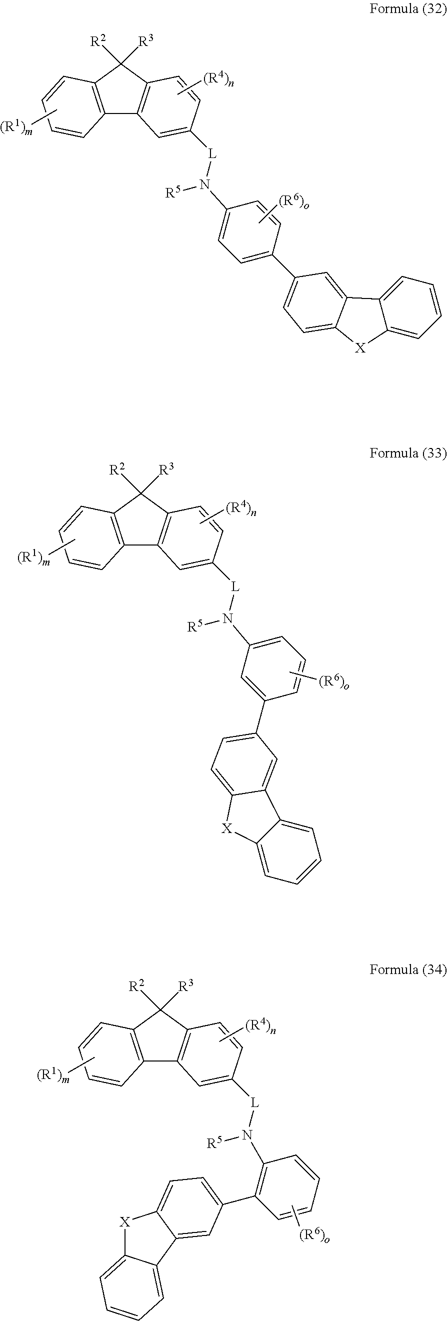

A preferred embodiment of the present invention relates to compounds of the formula (3), very preferably of the formula (7), particularly preferably of the formulae (11) to (13), very particularly preferably of the formulae (23) to (25), especially preferably of the formula (11) and most preferably of the formula (23), where the L group in the compounds mentioned has the formula (L-1), preferably one of the formulae (L-1-1) to (L-1-3) and very preferably the formula (L-1-1).

A further preferred embodiment of the present invention relates to compounds of the formula (4), very preferably those of the formula (8), particularly preferably those of the formulae (14) to (16), very particularly those of the formulae (26) to (28), especially preferably those of the formula (14) and most preferably those of the formula (26), where the L group in the compounds mentioned has the formula (L-1), preferably one of the formulae (L-1-1) to (L-1-3) and very preferably the formula (L-1-1).

A further preferred embodiment of the present invention relates to compounds of the formula (3) or (4), very preferably those of the formula (7) or (8), particularly preferably those of the formulae (11) to (16) and very particularly preferably those of the formulae (11) or (14), where the L group in the compounds mentioned has the formula (L-1), preferably one of the formulae (L-1-1) to (L-1-3) and very preferably the formula (L-1-1).

Yet a further preferred embodiment of the present invention relates to compounds of the formula (5), very preferably those of the formula (9), particularly preferably those of the formulae (17) to (19) and very particularly preferably those of the formulae (29) to (31).

And yet a further preferred embodiment of the present invention relates to compounds of the formula (6), very preferably those of the formula (10), particularly preferably those of the formulae (20) to (22) and very particularly preferably those of the formulae (32) to (34).

It is further preferred when, in the aforementioned compounds, the R² and R³ radicals are the same or different at each instance and are selected from straight-chain alkyl groups having 1 to 20 carbon atoms, branched or cyclic alkyl groups having 3 to 20 carbon atoms, aromatic ring systems having 6 to 40 aromatic ring atoms, and heteroaromatic ring systems having 5 to 40 aromatic ring atoms; where the two R² and R³ radicals may be joined to one another and may form a ring; where the alkyl groups mentioned and the aromatic ring systems and heteroaromatic ring systems mentioned may each be substituted by R¹¹ radicals; and where one or more CH₂ groups in the alkyl groups may be replaced by —R¹¹C═CR¹¹—, —C≡C—, Si(R¹¹)₂, C═O, C═NR¹¹, —C(═O)O—, —C(═O)NR¹¹—, NR¹¹, P(═O)(R¹¹), —O—, —S—, SO or SO₂; if the two R² and R³ radicals form a ring, the result is a spiro compound, preferably a spirobifluorene; it is particularly preferable when the two R² and R³ radicals do not form a ring with one another.

It is even more preferred in the context of the present invention when, in the aforementioned compounds, the R² and R³ radicals are the same or different at each instance and are selected from straight-chain alkyl groups having 1 to 10 carbon atoms, branched or cyclic alkyl groups having 3 to 10 carbon atoms, aromatic ring systems having 6 to 18 aromatic ring atoms, and heteroaromatic ring systems having 5 to 18 aromatic ring atoms; where the two R² and R³ radicals may be joined to one another and may form a ring; where the alkyl groups mentioned and the aromatic ring systems and heteroaromatic ring systems mentioned may each be substituted by R¹¹ radicals; and where one or more CH₂ groups in the alkyl groups may be replaced by —R¹¹C═CR¹¹—, —C≡C—, Si(R¹¹)₂, C═O, C═NR¹¹, —C(═O)O—, —C(═O)NR¹¹—, NR¹¹, P(═O)(R¹¹), —O—, —S—, SO or SO₂; if the two R² and R³ radicals form a ring, the result is a spiro compound, preferably a spirobifluorene; it is particularly preferable when the two R² and R³ radicals do not form a ring with one another.

It is even more preferred in the context of the present invention when, in the aforementioned compounds, the R² and R³ radicals are the same or different at each instance and are selected from straight-chain alkyl groups having 1 to 10 carbon atoms, branched or cyclic alkyl groups having 3 to 10 carbon atoms or aromatic ring systems having 6 to 18 aromatic atoms; where the two R² and R³ radicals may be joined to one another and may form a ring; where the alkyl groups mentioned and the aromatic ring systems mentioned may each be substituted by R¹¹ radicals; if the two R² and R³ radicals form a ring, the result is a spiro compound, preferably a spirobifluorene; it is particularly preferable when the two R² and R³ radicals do not form a ring with one another.

It is especially preferable in the context of the present invention when, in the aforementioned compounds, the R² and R³ radicals are the same or different at each instance and are selected from straight-chain alkyl groups having 1 to 10 carbon atoms, branched or cyclic alkyl groups having 3 to 10 carbon atoms and aromatic ring systems having 6 to 18 aromatic ring atoms; where the alkyl groups mentioned and the aromatic ring systems mentioned may each be substituted by R¹¹ radicals; preferably, the two R² and R³ radicals are in unsubstituted form.

It is also especially preferable in the context of the present invention when, in the aforementioned compounds, the R² and R³ radicals are the same or different at each instance and are selected from straight-chain alkyl groups having 1 to 10 carbon atoms and aromatic ring systems having 6 to 18 aromatic ring atoms; where the alkyl groups mentioned and the aromatic ring systems mentioned may each be substituted by R¹¹ radicals; preferably, the two R² and R³ radicals are in unsubstituted form.

It is very particularly preferable in the context of the present invention when, in the aforementioned compounds, the R² and R³ radicals are the same or different at each instance and are selected from straight-chain alkyl groups having 1 to 5 carbon atoms, where methyl groups are the most preferred.

It is very particularly preferable in the context of the present invention when, in the aforementioned compounds, the R² and R³ radicals are the same or different at each instance and are selected from aromatic ring systems having 6 to 12 aromatic ring atoms; where phenyl groups are the most preferred.

In a preferred embodiment of the present invention, R² and R³ are the same.

In yet another preferred embodiment of the present invention, R² and R³ are different.

R⁵ radicals are preferably selected from monovalent groups derived from benzene, biphenyl, terphenyl, quaterphenyl, naphthalene, fluorene, especially 9,9′-dimethylfluorene and 9,9′-diphenylfluorene, 9-silafluorene, especially 9,9′-dimethyl-9-silafluorene and 9,9′-diphenyl-9-silafluorene, benzofluorene, spirobifluorene, indenofluorene, indenocarbazole, dibenzofuran, dibenzothiophene, benzocarbazole, carbazole, benzofuran, benzothiophene, indole, quinoline, pyridine, pyrimidine, pyrazine, pyridazine and triazine, where the monovalent groups are each substituted by one or more R¹¹ radicals. Alternatively, the R⁵ group may preferably be selected from combinations of groups derived from benzene, biphenyl, terphenyl, quaterphenyl, naphthalene, fluorene, especially 9,9′-dimethylfluorene and 9,9′-diphenylfluorene, 9-silafluorene, especially 9,9′-dimethyl-9-silafluorene and 9,9′-diphenyl-9-silafluorene, benzofluorene, spirobifluorene, indenofluorene, indenocarbazole, dibenzofuran, dibenzothiophene, carbazole, benzofuran, benzothiophene, indole, quinoline, pyridine, pyrimidine, pyrazine, pyridazine and triazine, where the groups are each substituted by one or more R¹¹ radicals.

Particularly preferred R⁵ groups are selected from phenyl, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, and triazinyl-substituted phenyl, where the groups mentioned are each substituted by one or more R¹¹ radicals.

Particularly preferred groups for R⁵ are selected from the following formulae:

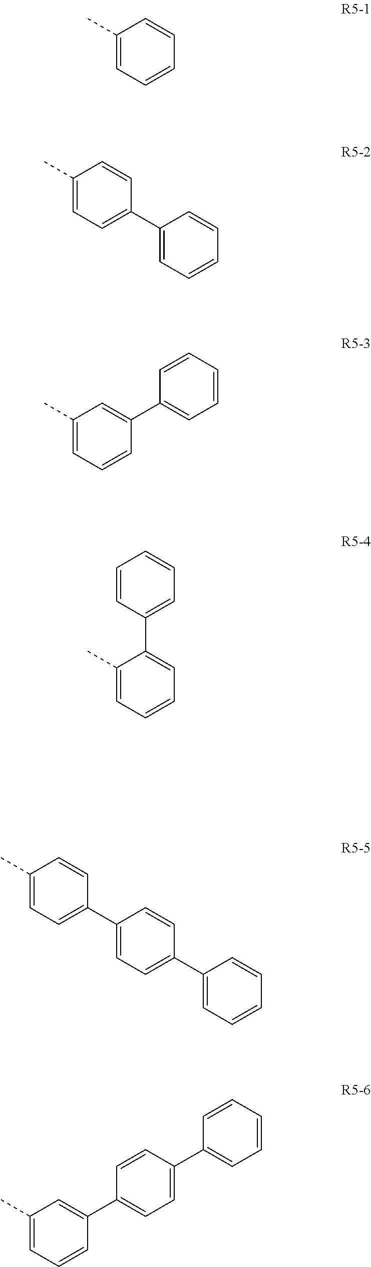

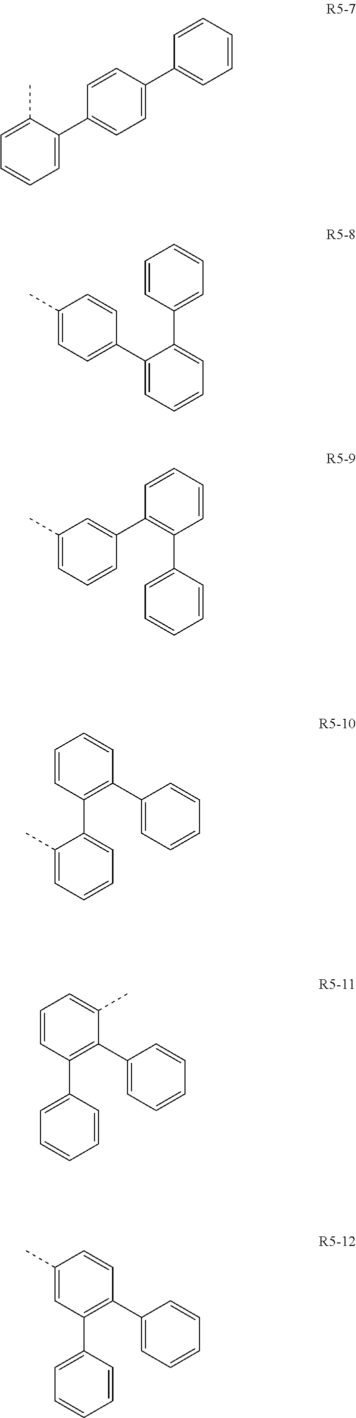

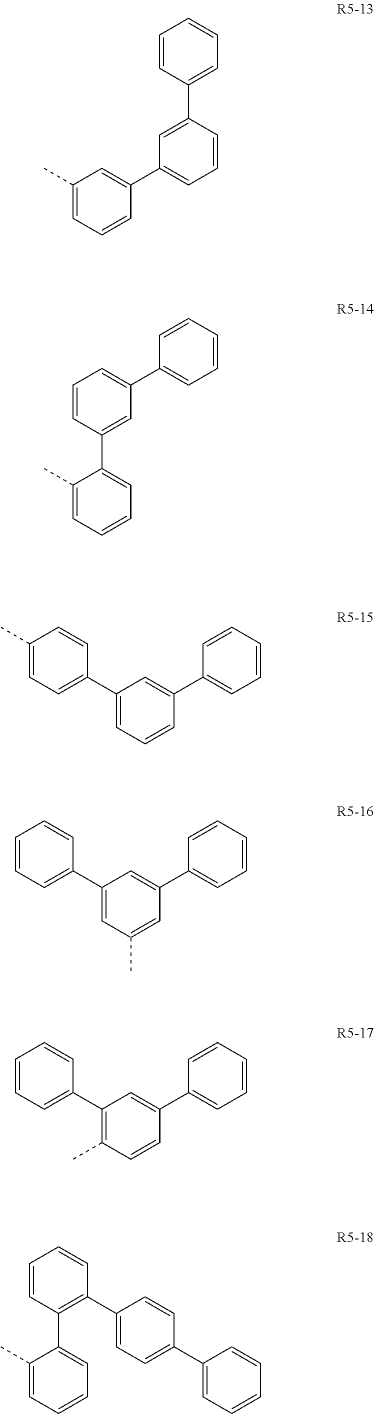

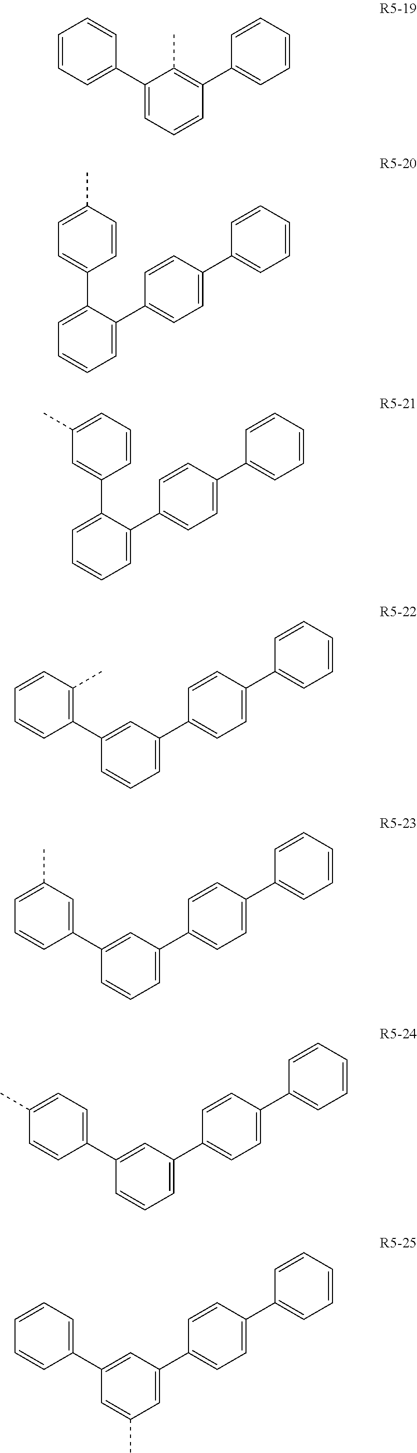

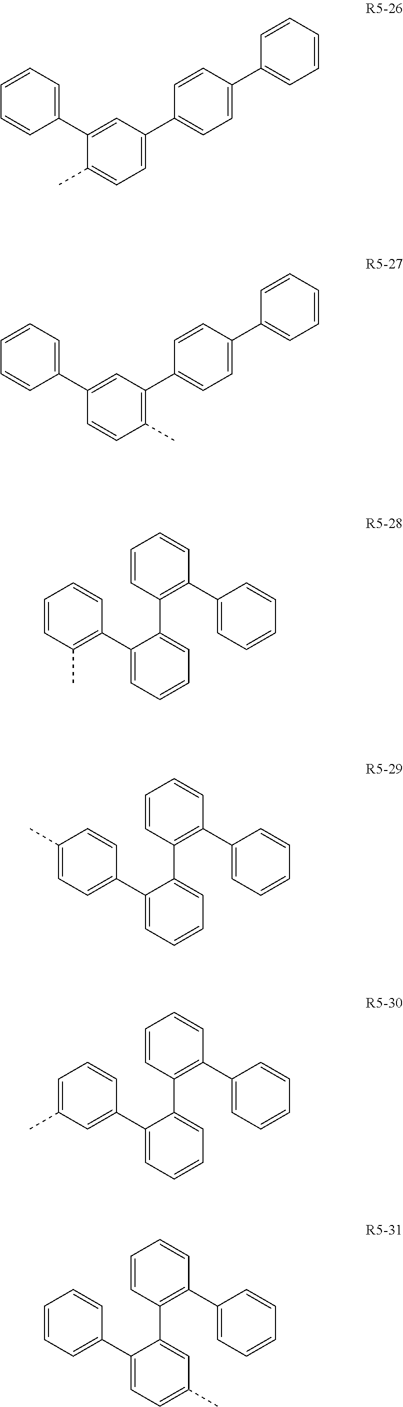

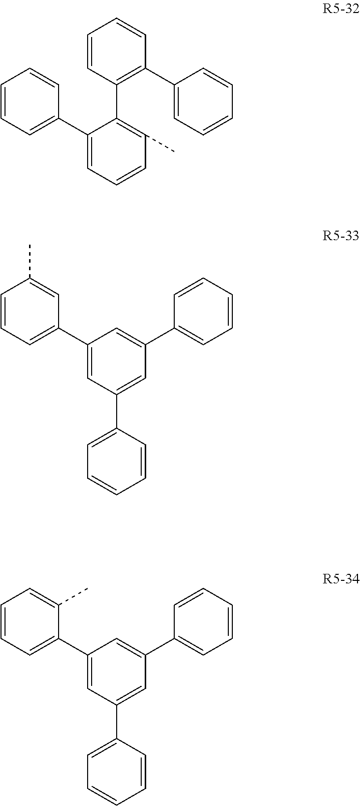

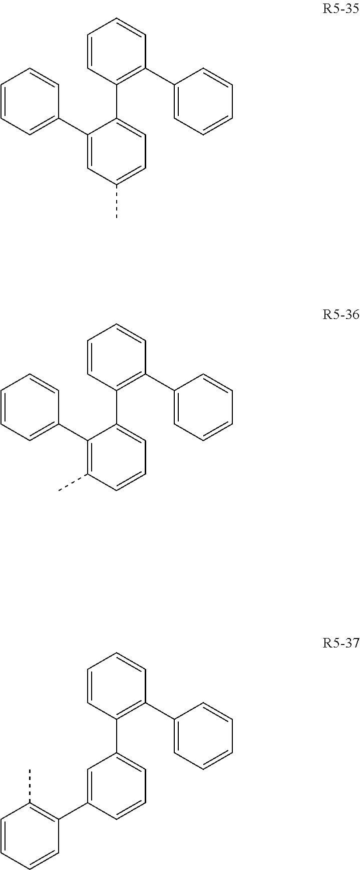

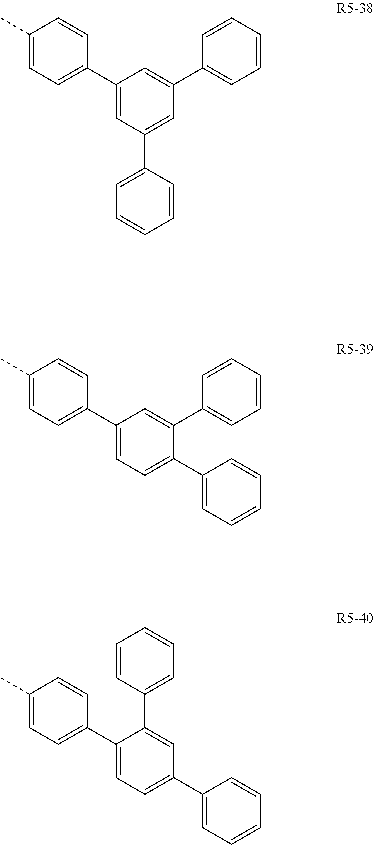

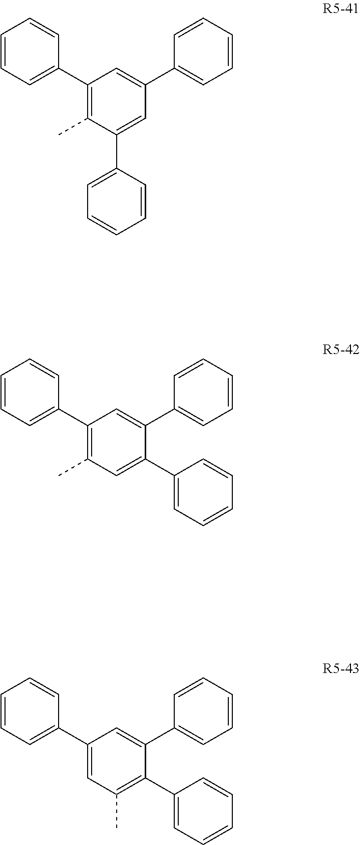

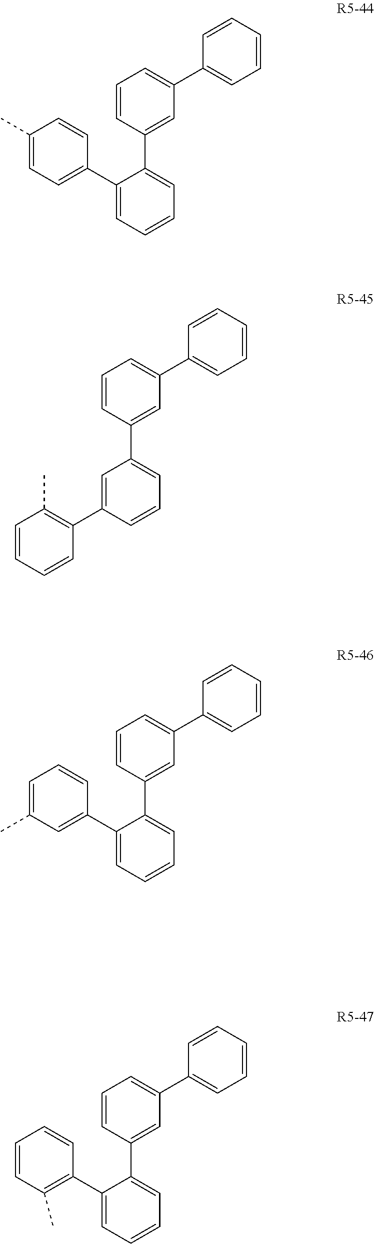

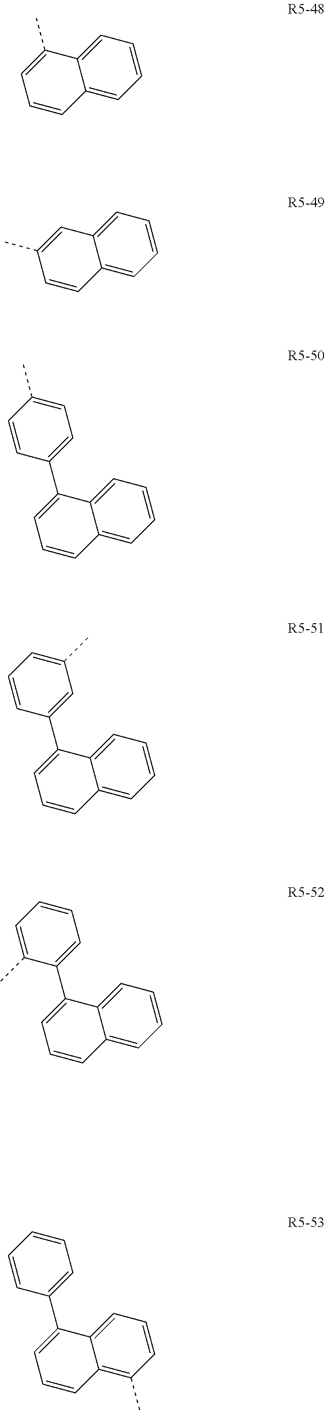

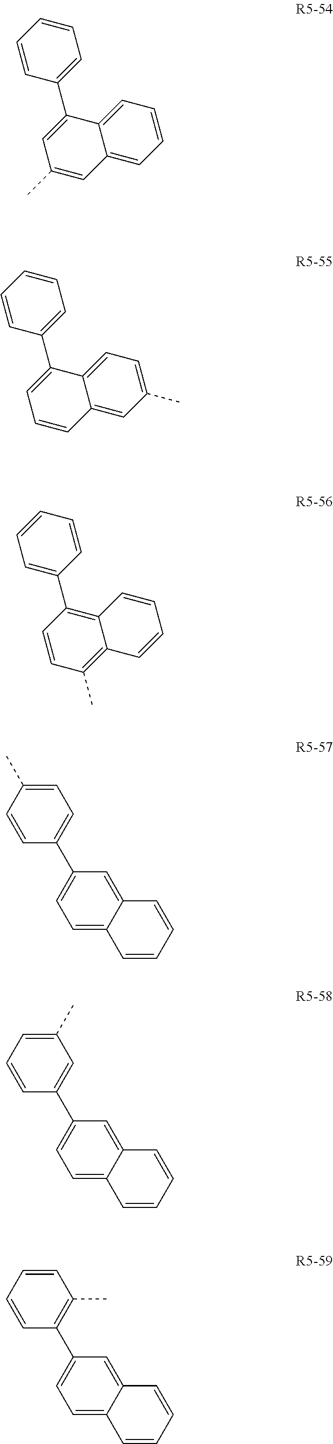

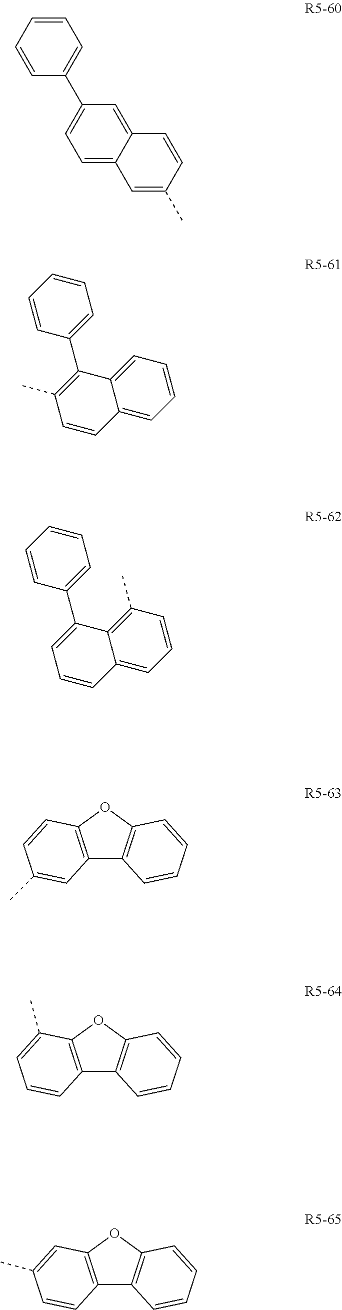

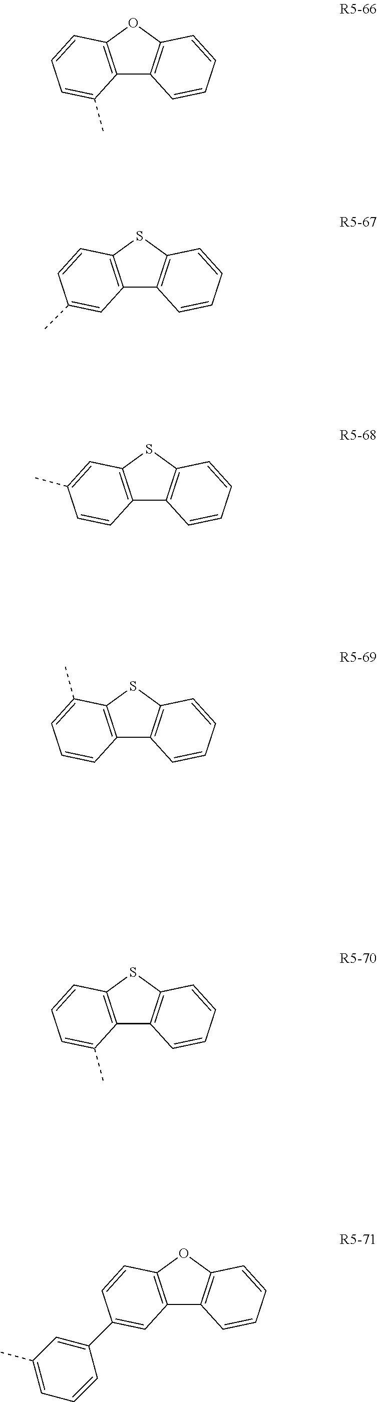

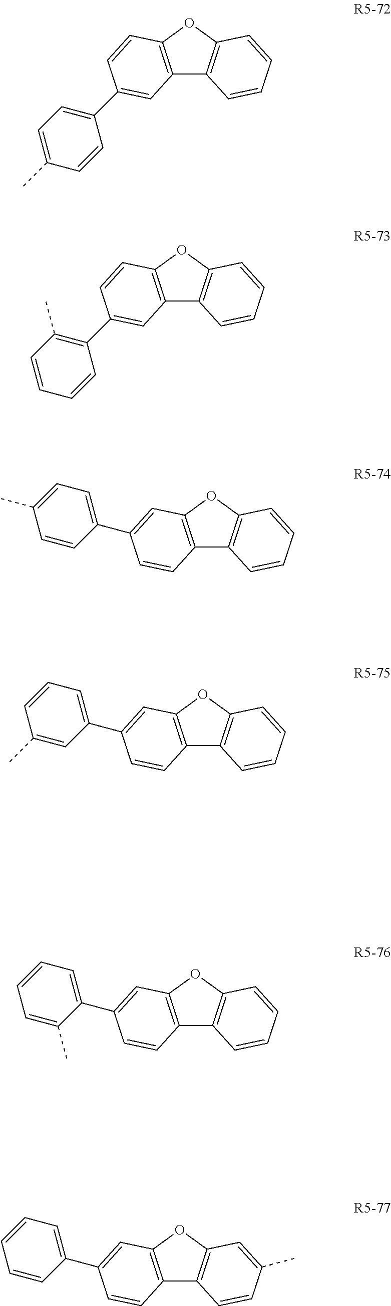

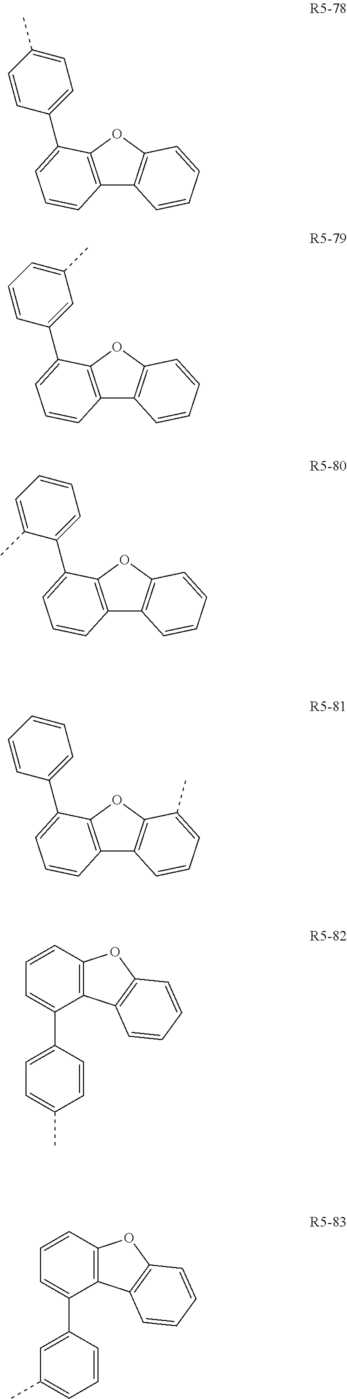

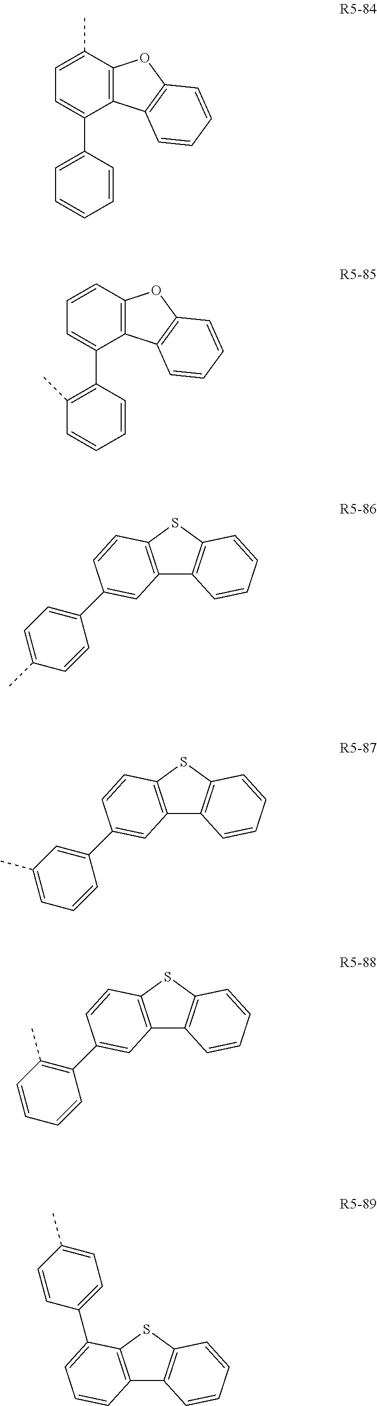

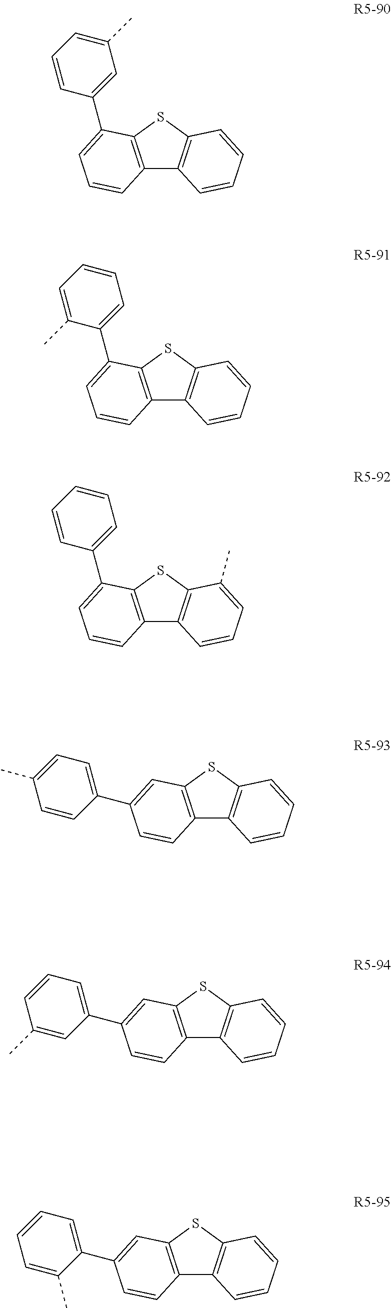

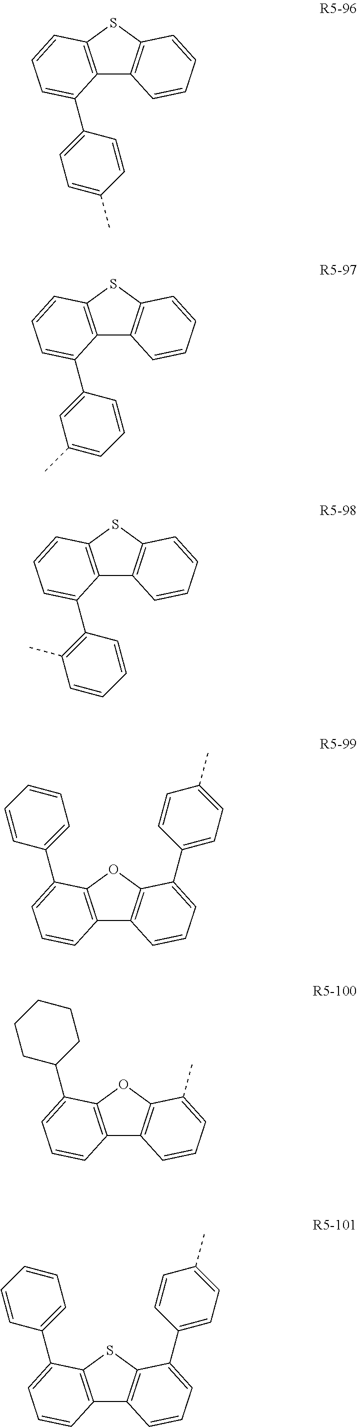

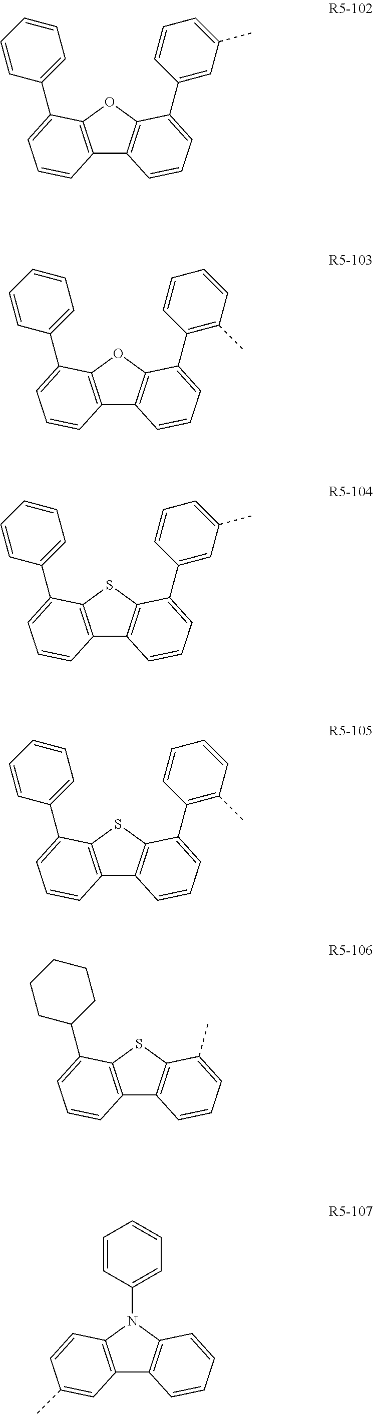

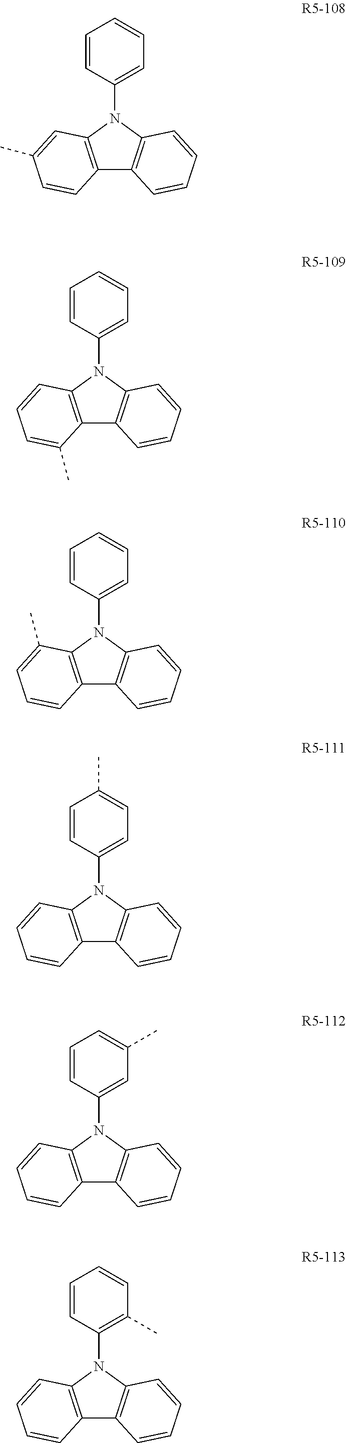

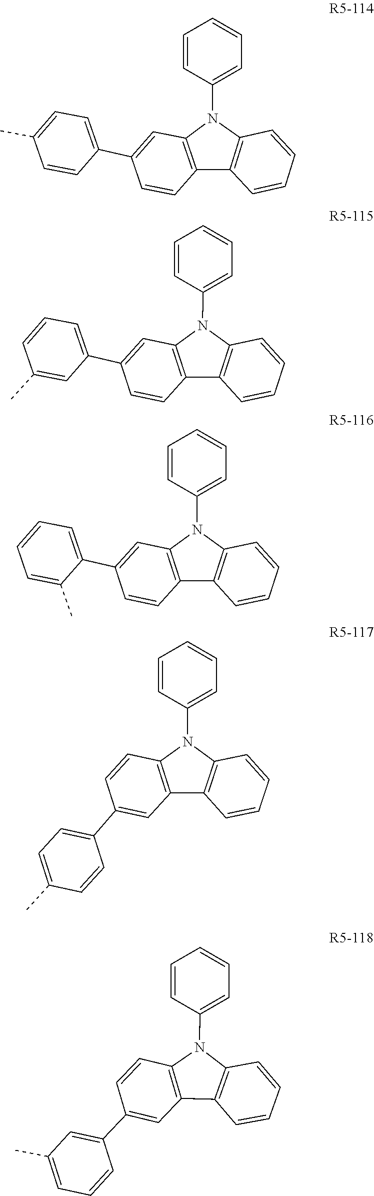

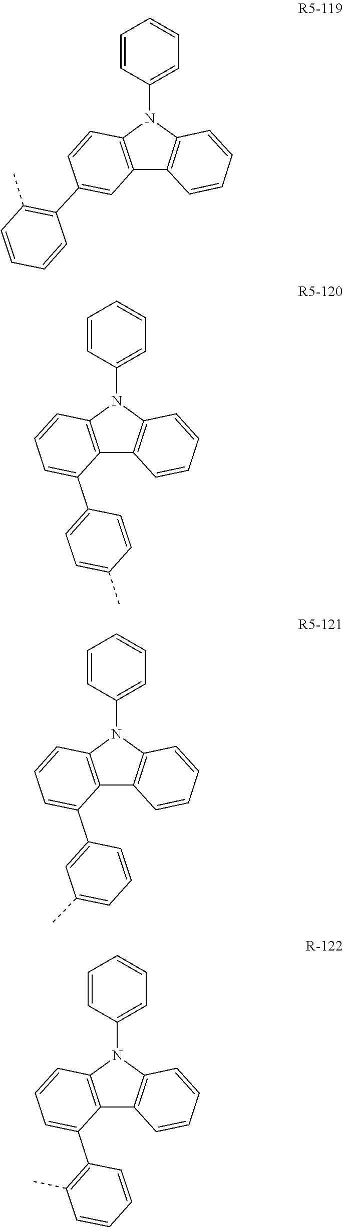

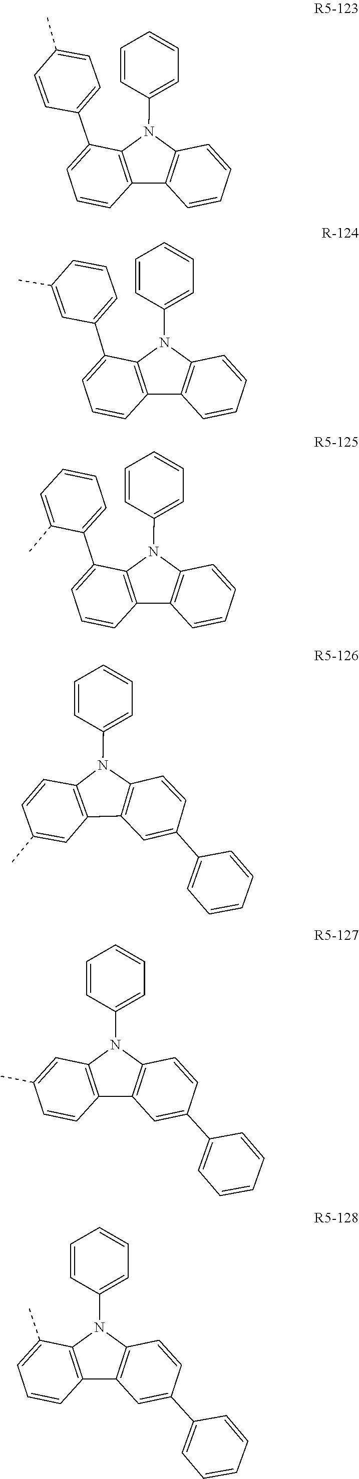

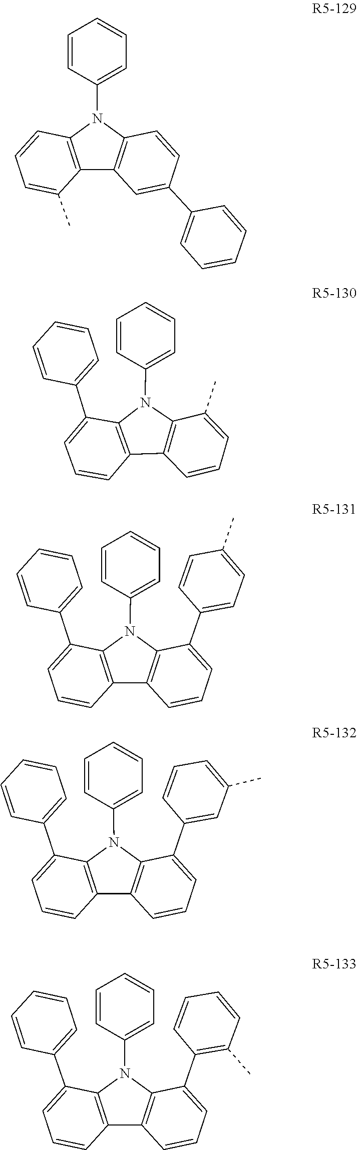

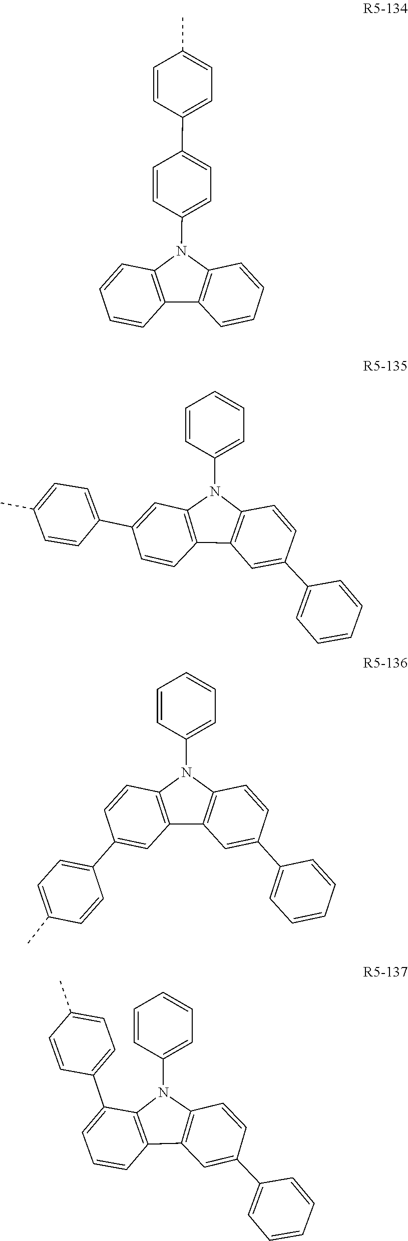

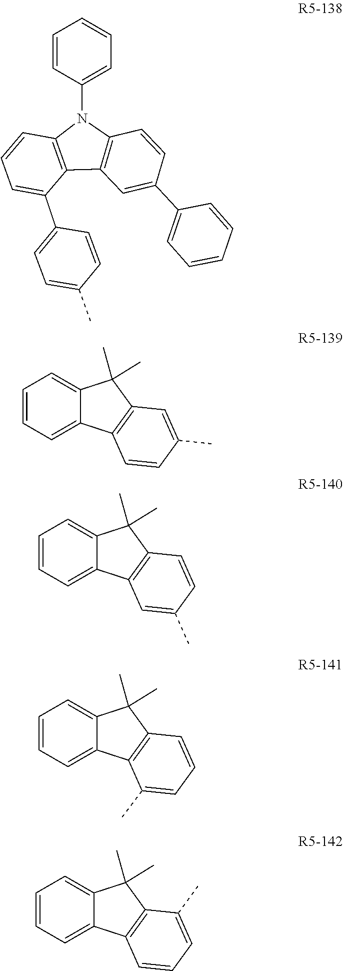

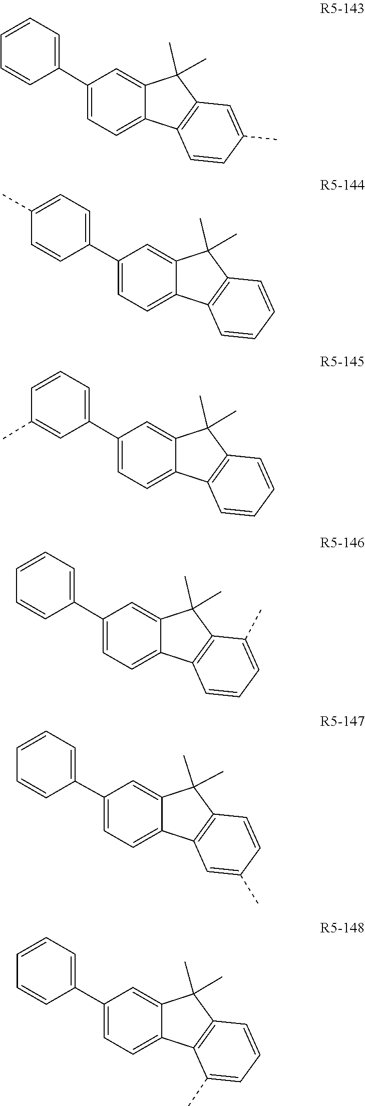

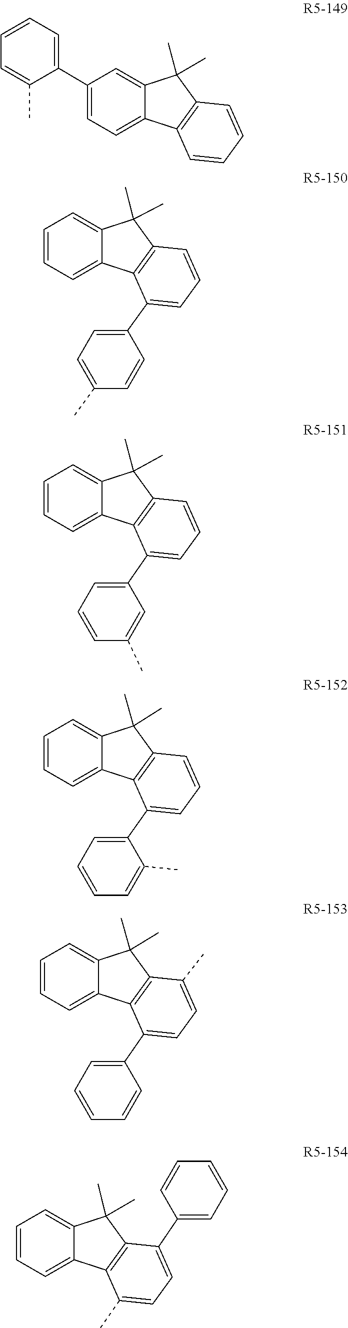

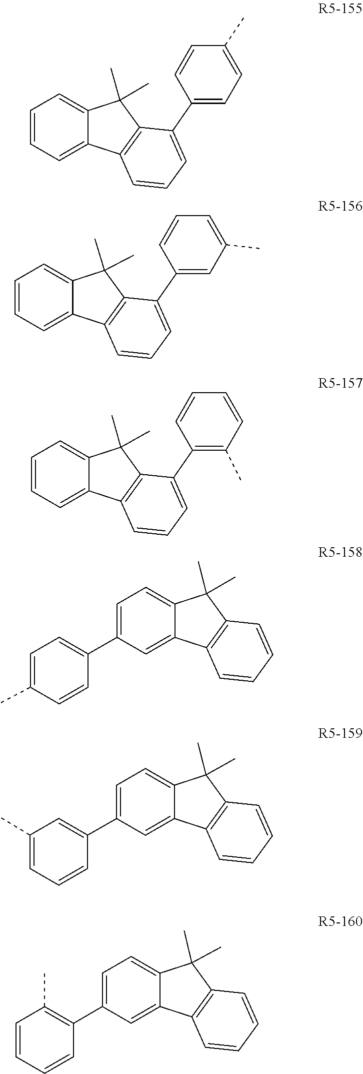

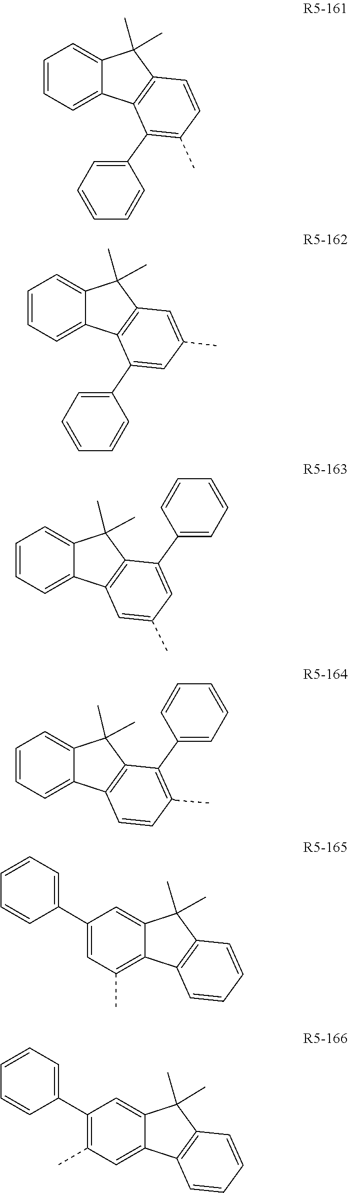

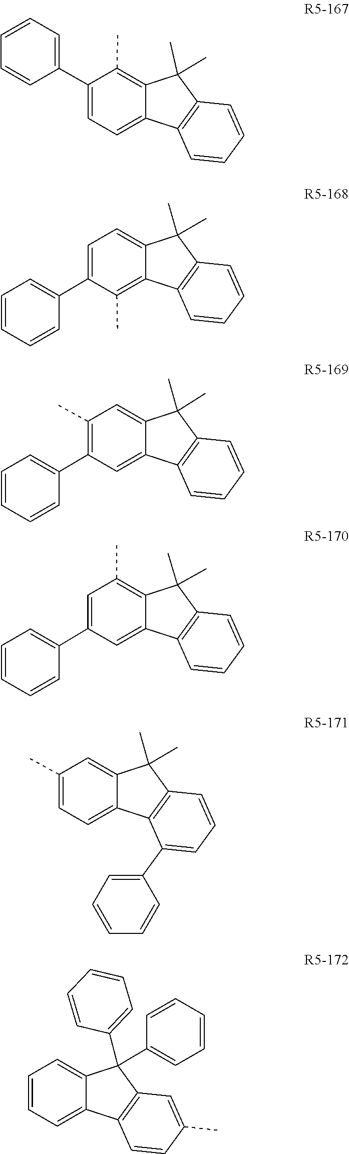

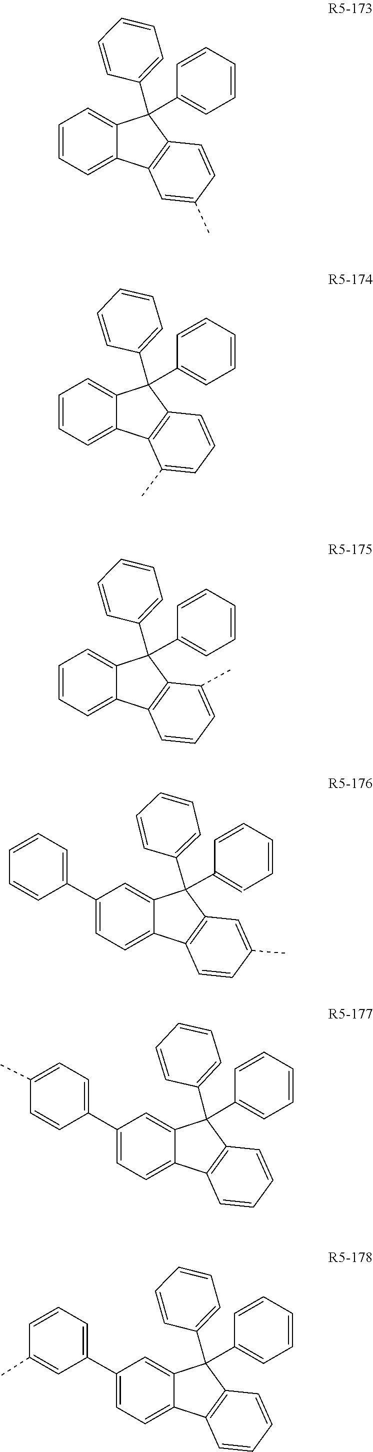

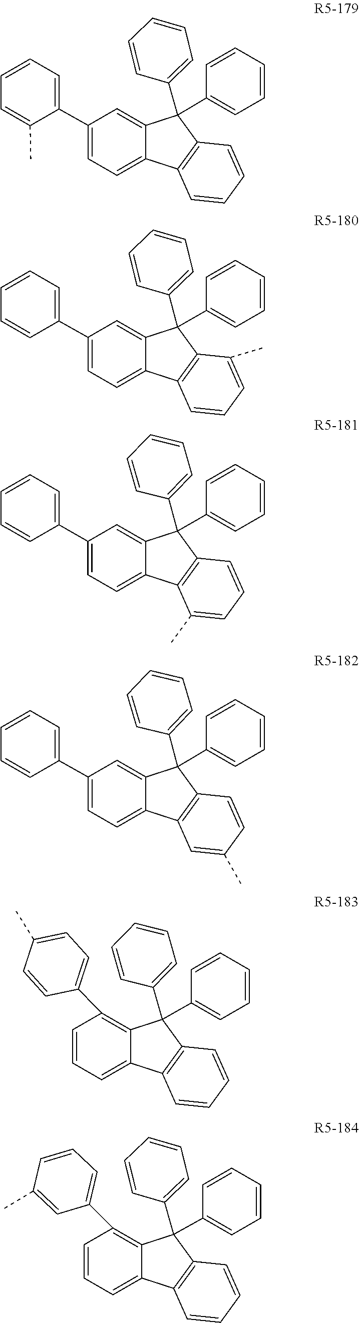

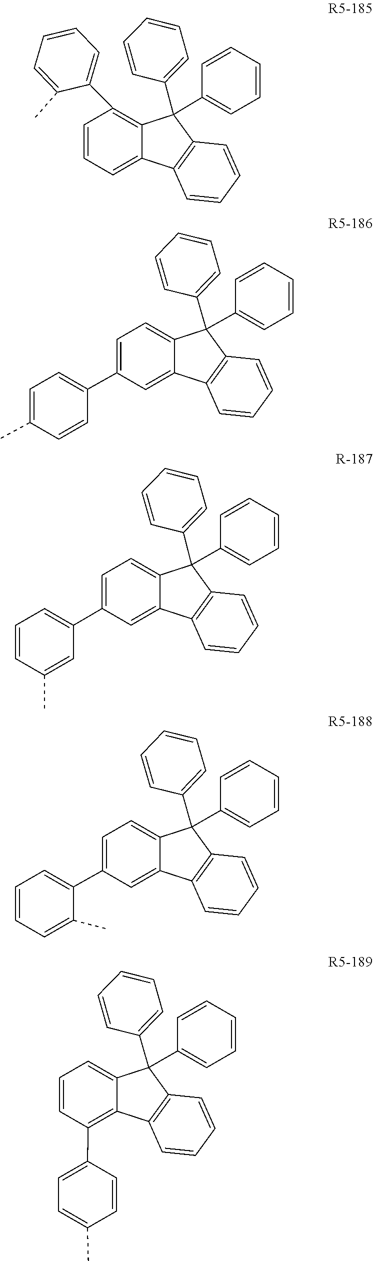

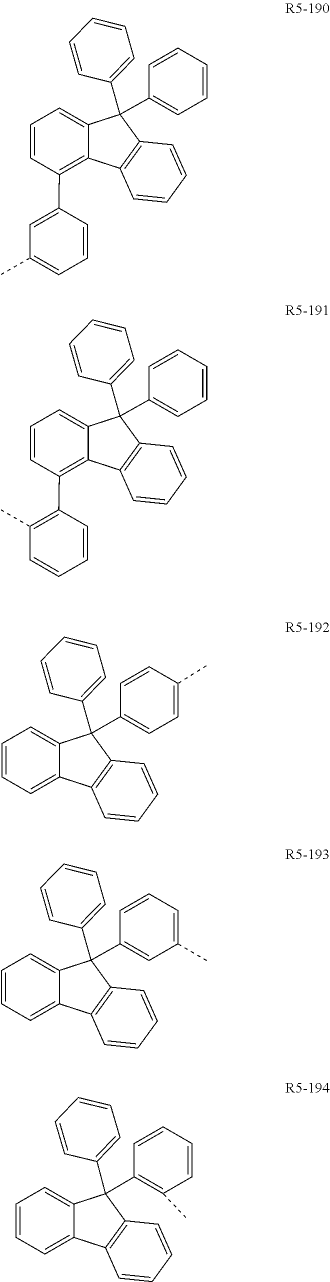

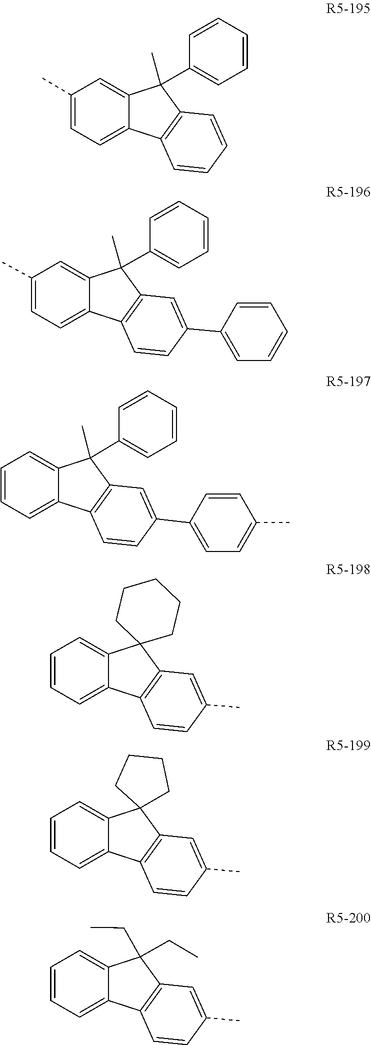

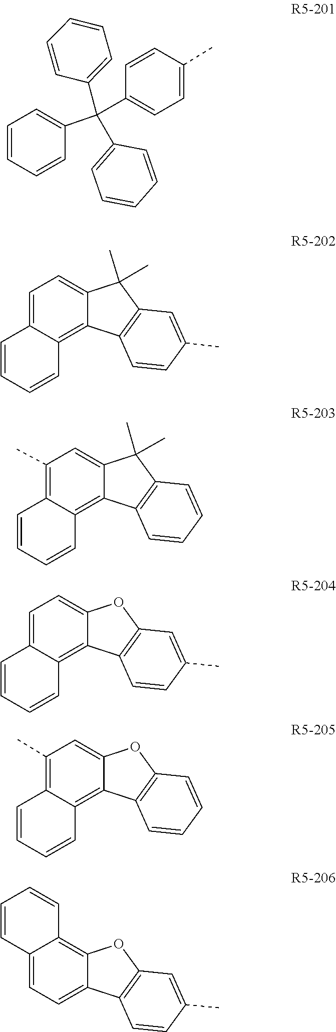

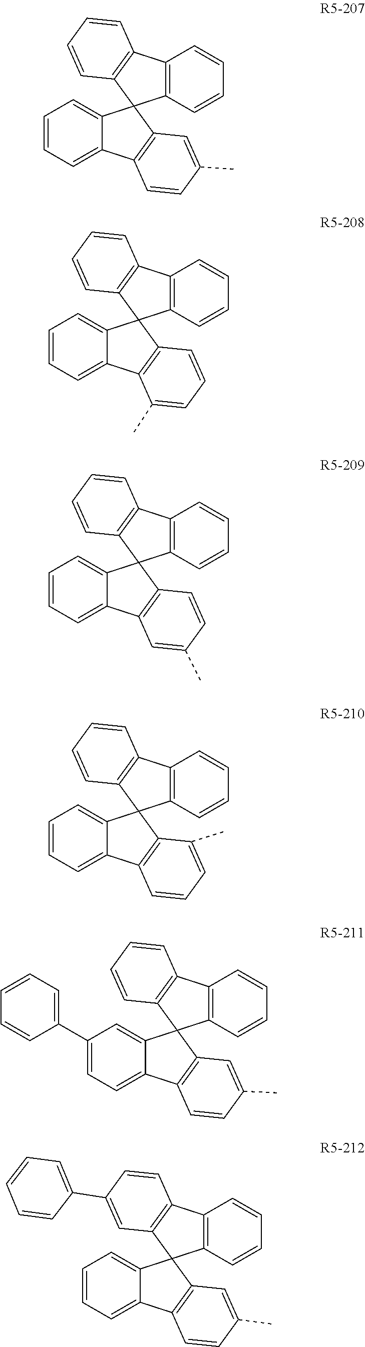

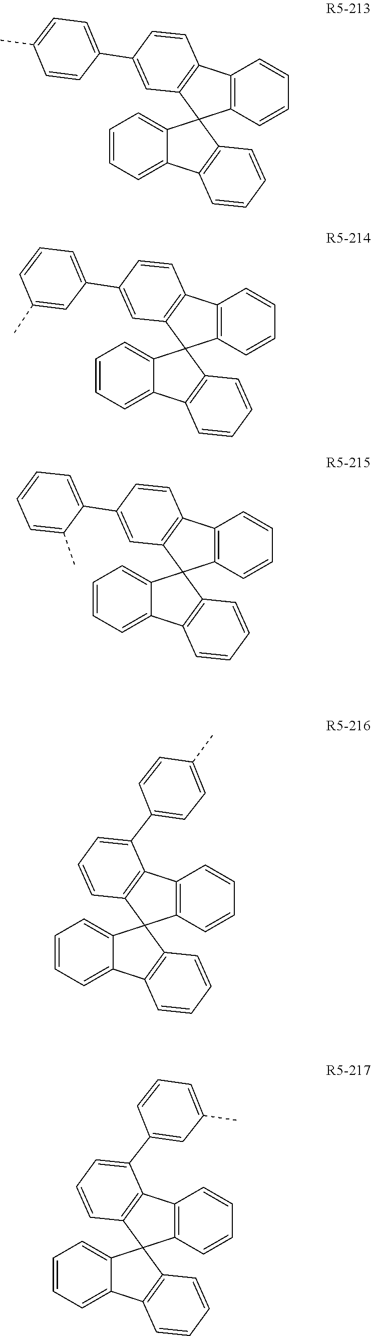

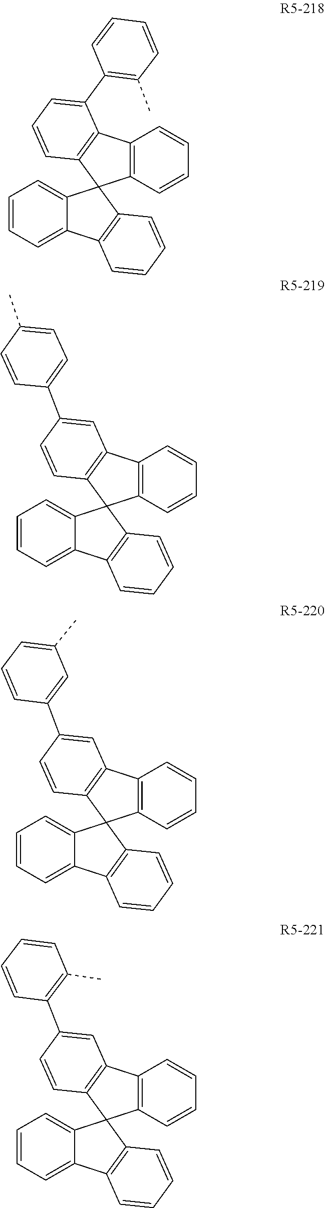

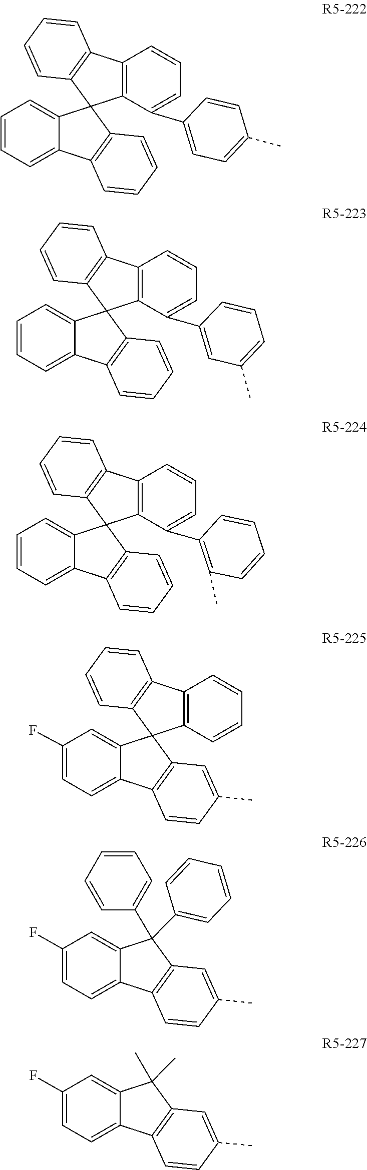

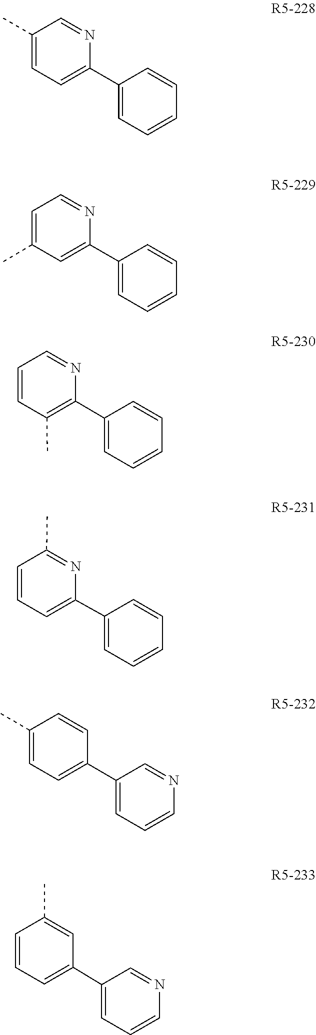

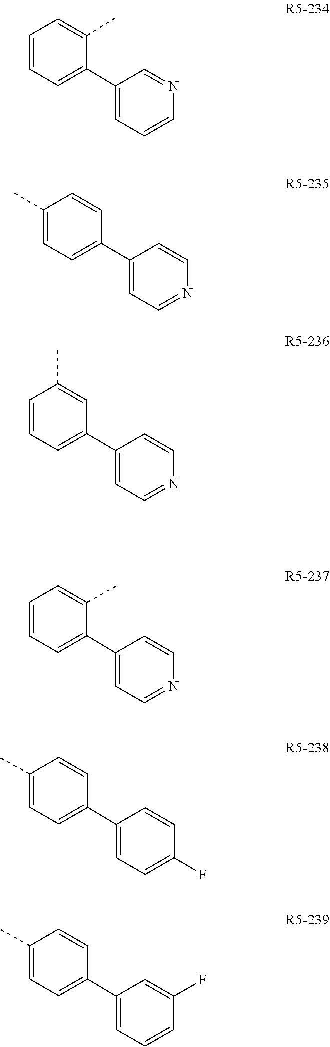

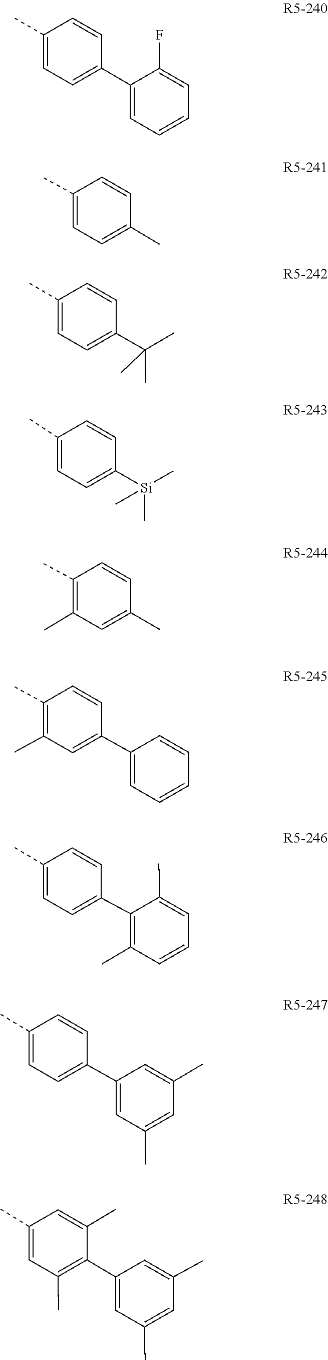

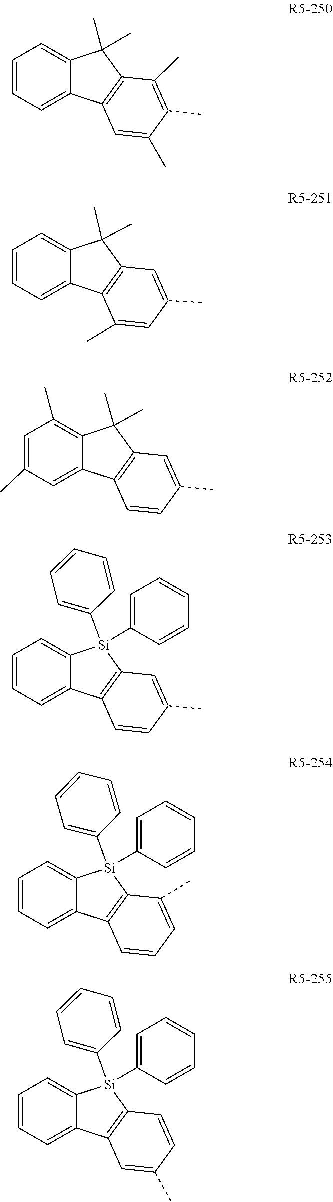

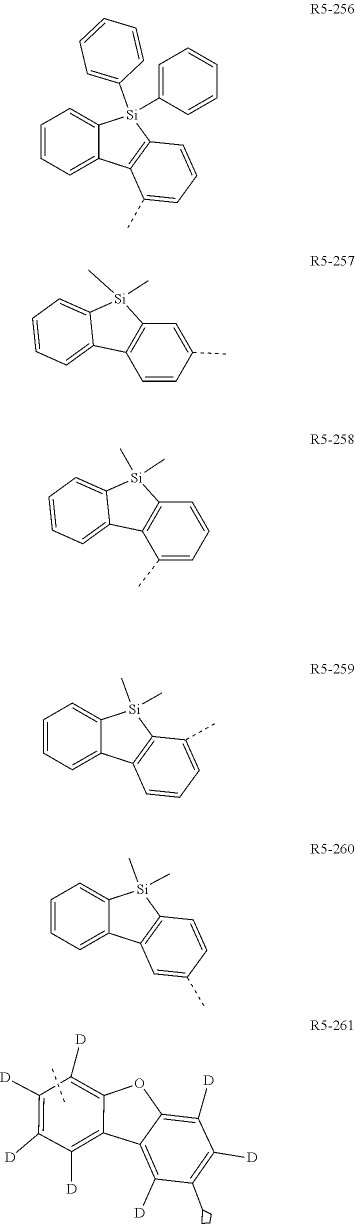

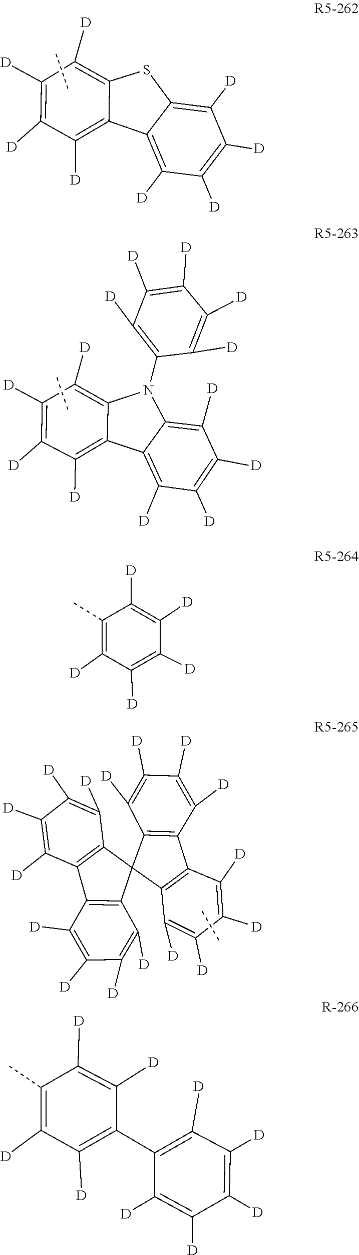

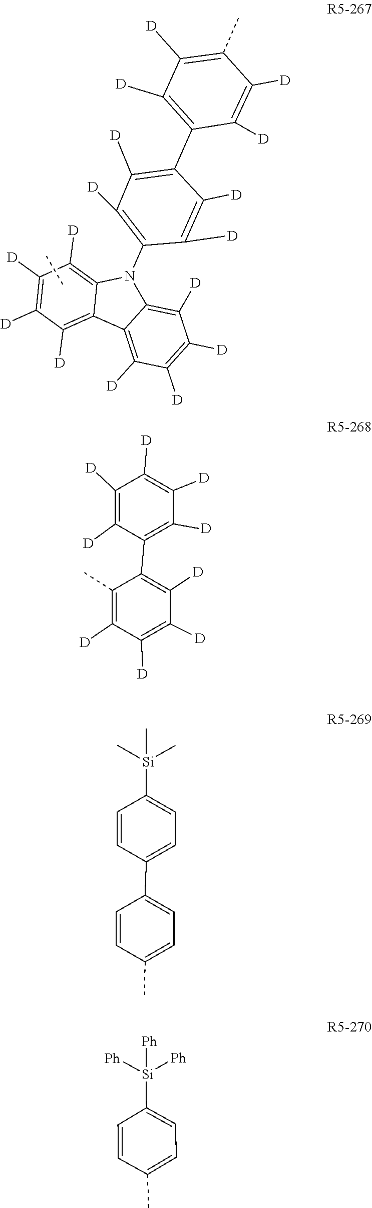

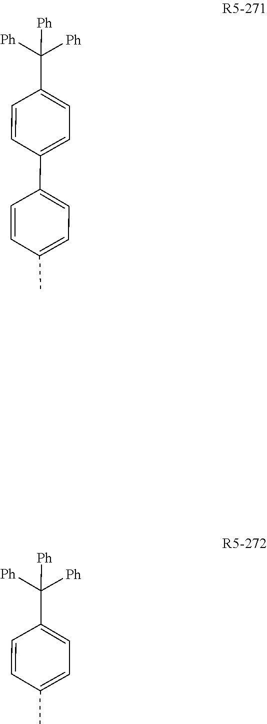

where the groups at the positions shown as unsubstituted may be substituted by R¹¹ radicals, where R¹¹ in these positions is preferably H, and where the dotted bond is the bond to the amine nitrogen atom.

It is further preferable when the abovementioned compounds of the invention having the formulae mentioned and the preferred compounds described in the various embodiments have just one R¹ radical and preferably have no R¹ radical.

It is further preferable when the abovementioned compounds of the invention having the formulae mentioned and the preferred compounds described in the various embodiments have just one R⁴ radical and preferably have no R⁴ radical.

It is further preferable when the abovementioned compounds of the invention having the formulae mentioned and the preferred compounds described in the various embodiments have just one R⁶ radical and preferably have no R⁶ radical.

It is further preferable when the abovementioned compounds of the invention having the formulae mentioned and the preferred compounds described in the various embodiments have just one R⁷ radical and preferably have no R⁷ radical.

Finally, it is even more preferable when the abovementioned compounds of the invention having the formulae mentioned and the preferred compounds described in the various embodiments have just one R¹ radical and just one R⁴ radical and just one R⁸ radical and just one R⁷ radical, and the compounds preferably have none of the R¹, R⁴, R⁶ and R⁷ radicals.

In a preferred embodiment, X in the abovementioned compounds of the invention having the formulae mentioned and in the preferred compounds that are described in the various embodiments is O. Electronic devices comprising these compounds have very good lifetimes. Particularly good lifetimes can be observed in green-emitting organic electronic devices. In another preferred embodiment, X in the abovementioned compounds of the invention having the formulae mentioned and in the preferred compounds that are described in the various embodiments is S.

Preferred embodiments of inventive compounds of the formula (1) are shown below:

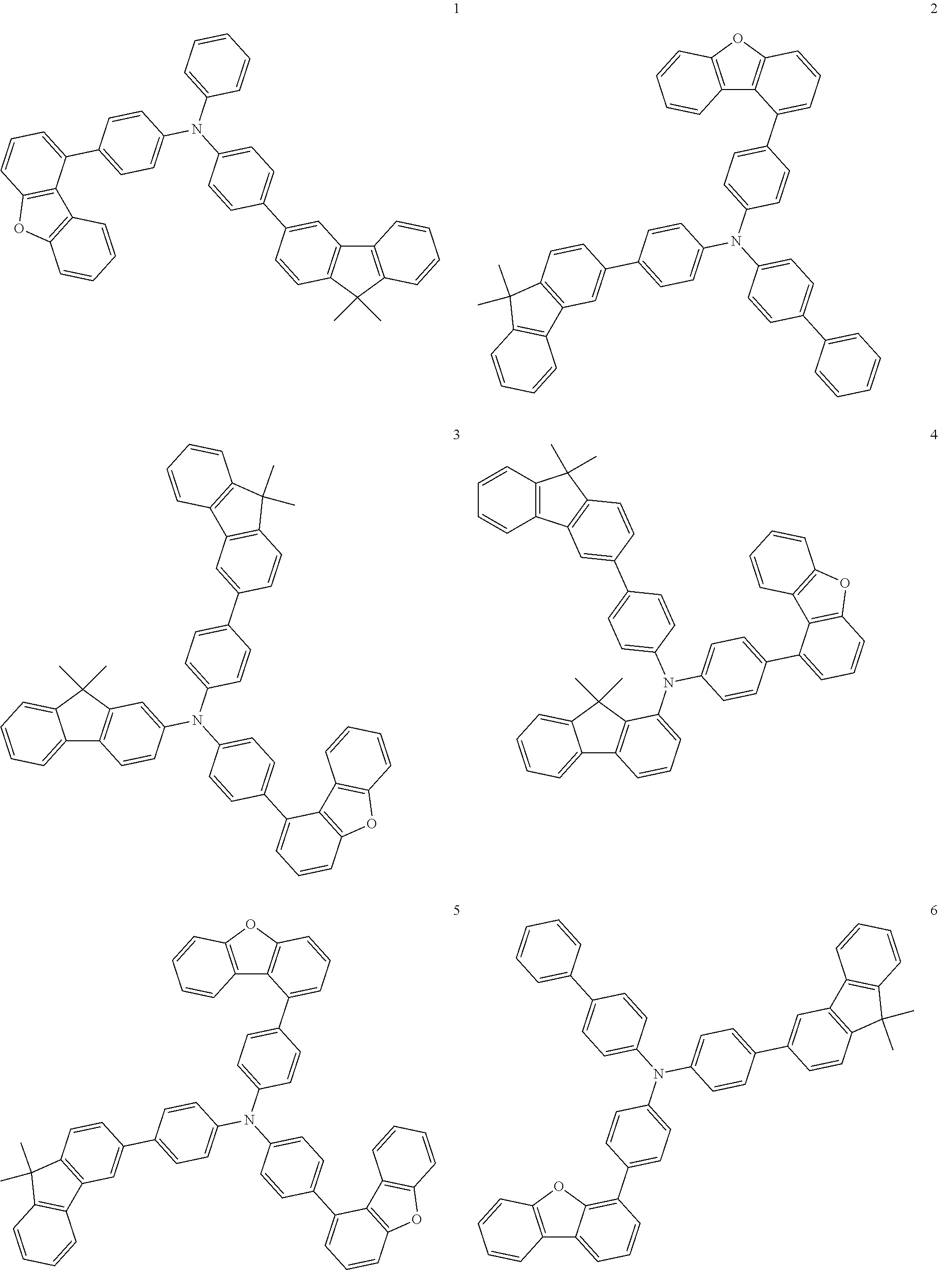

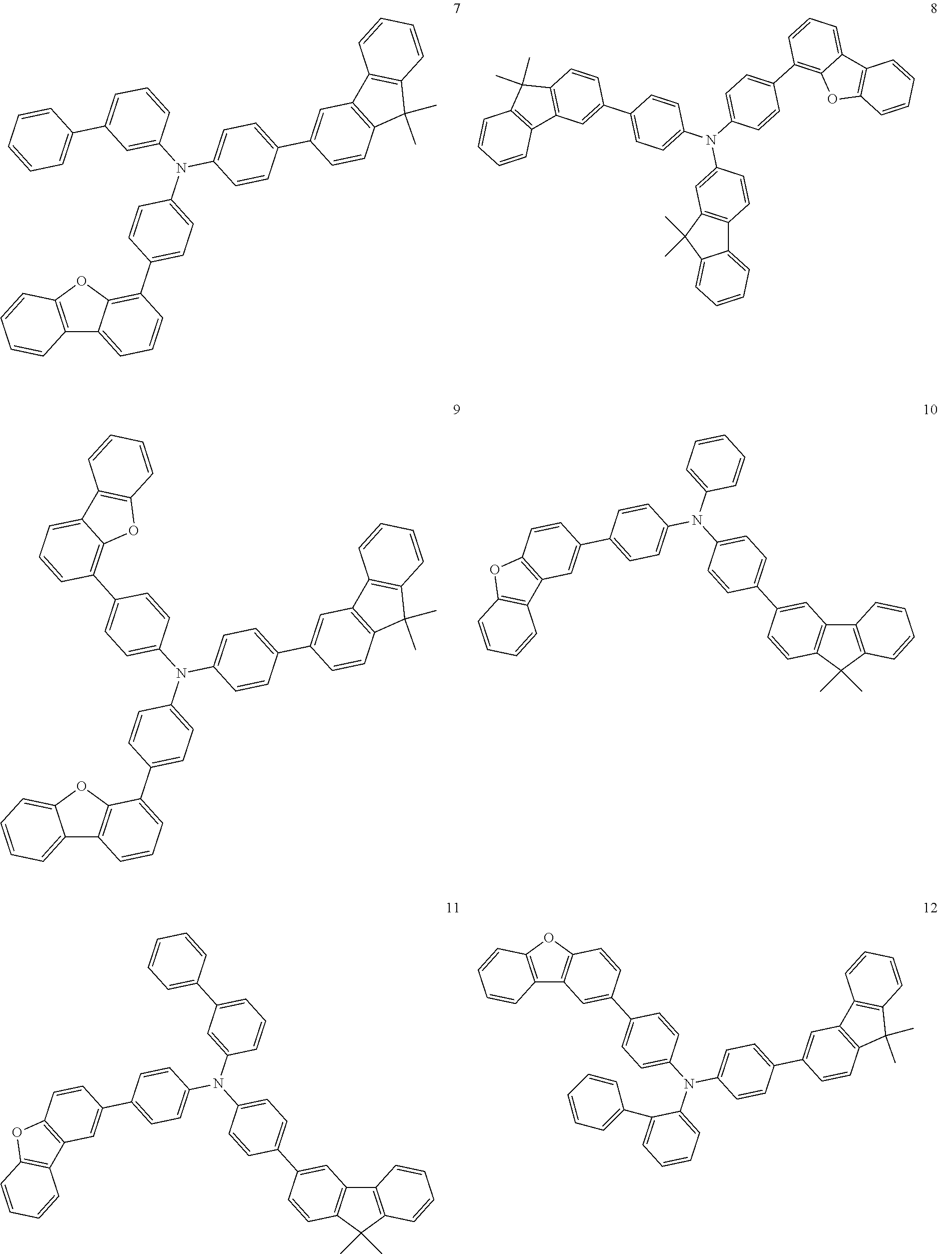

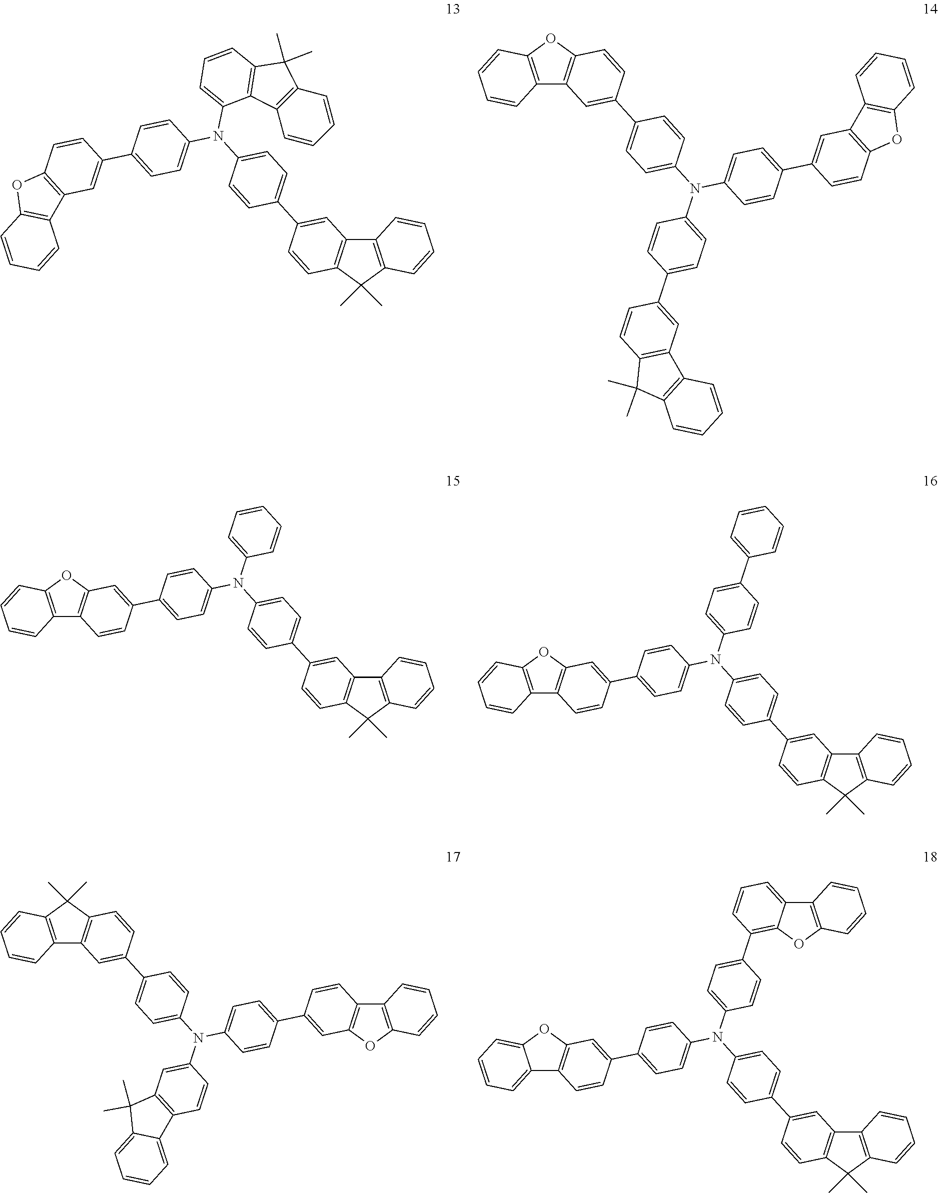

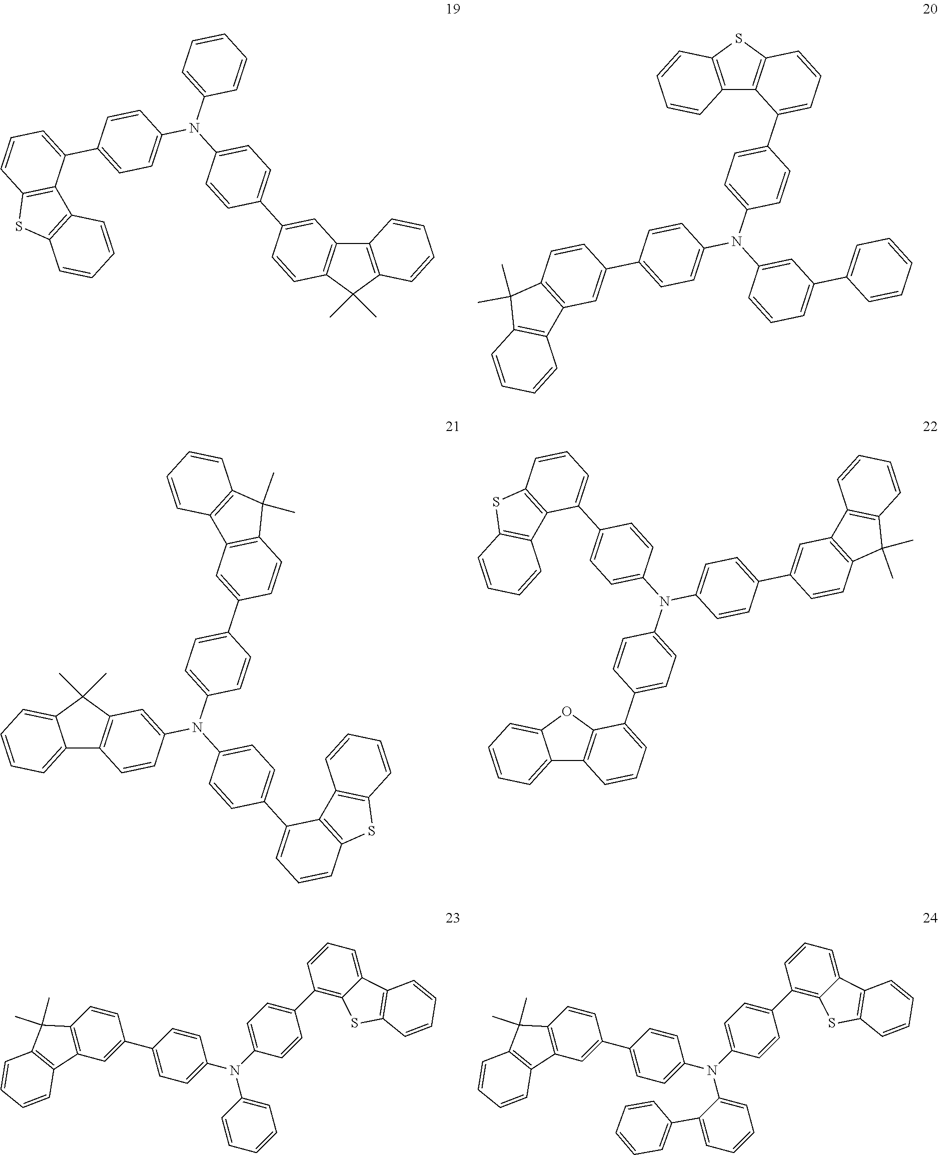

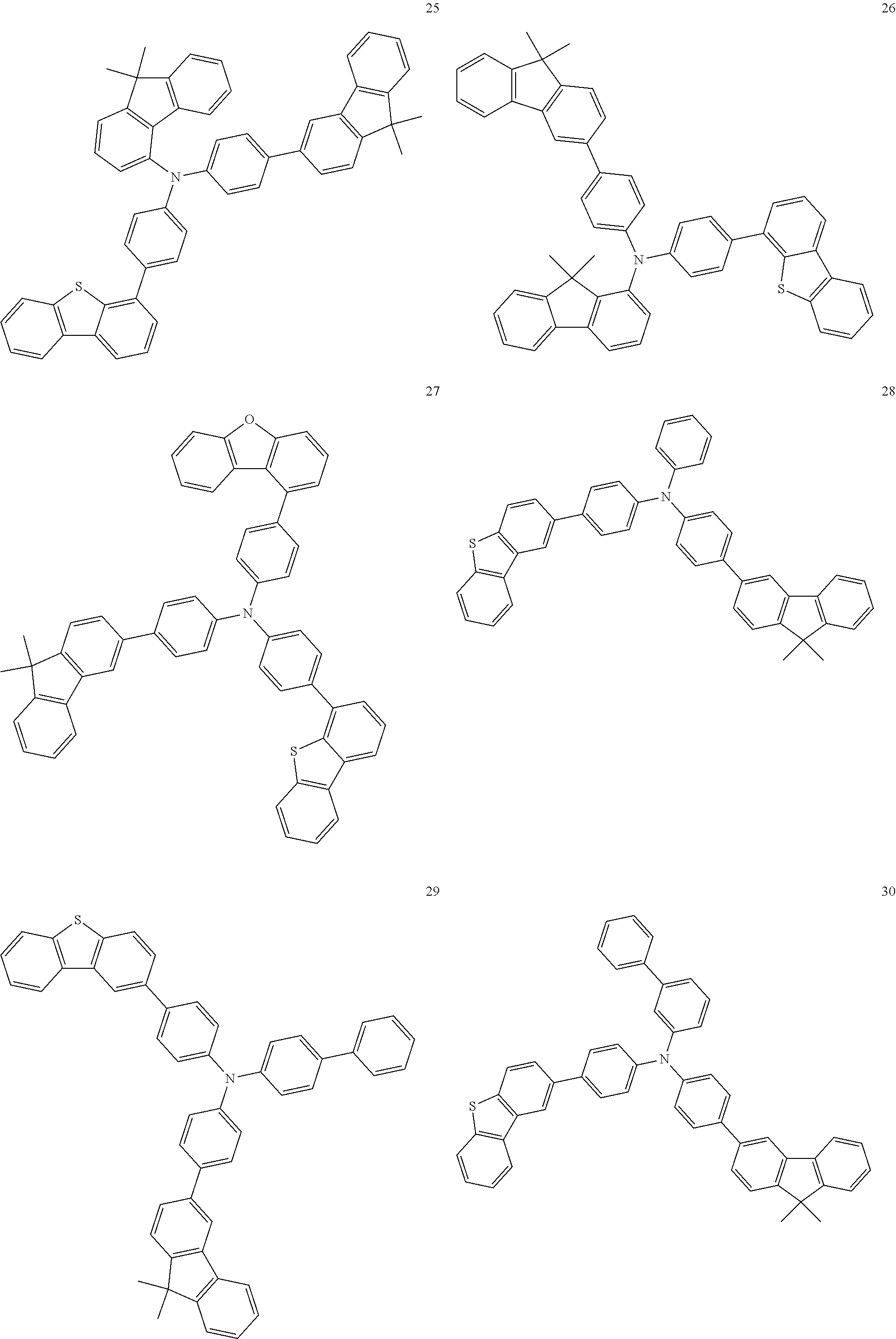

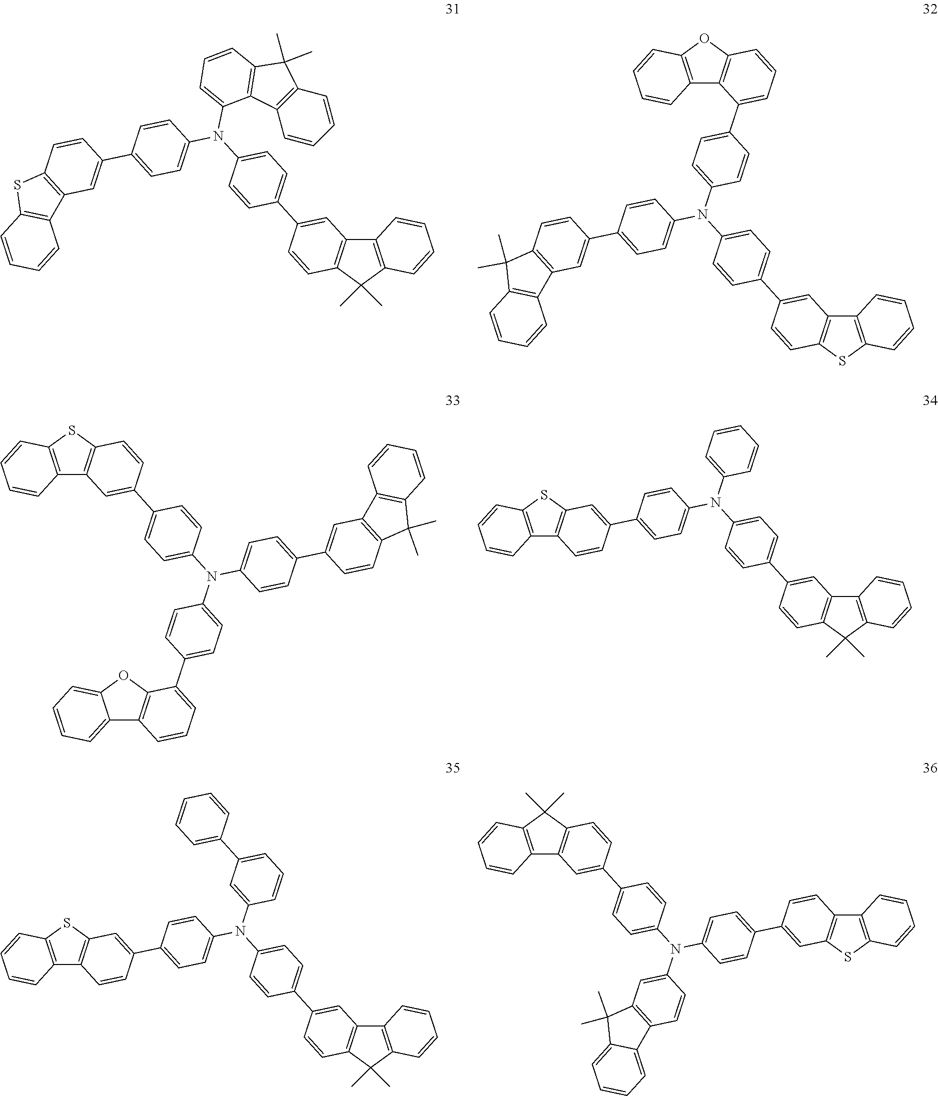

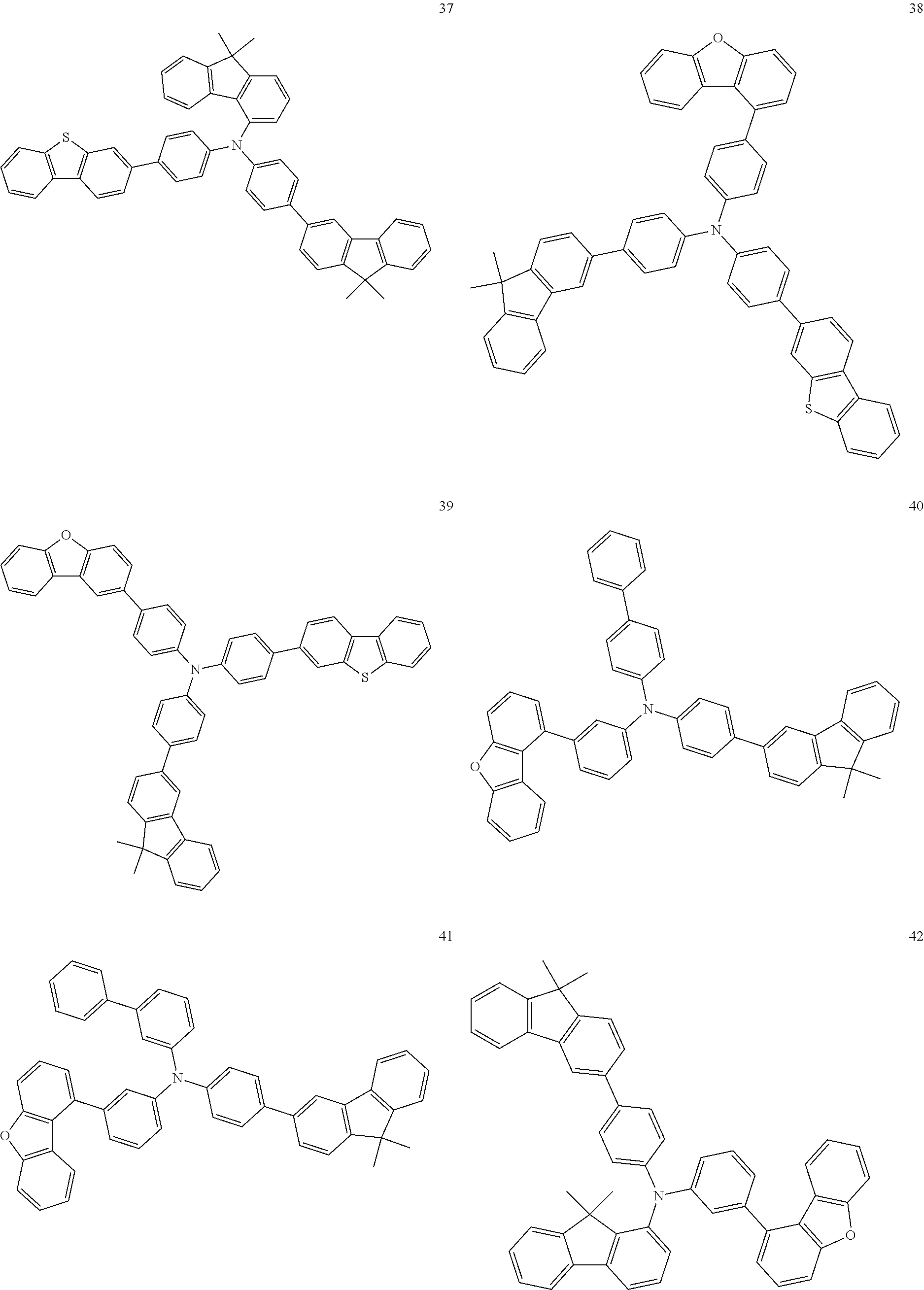

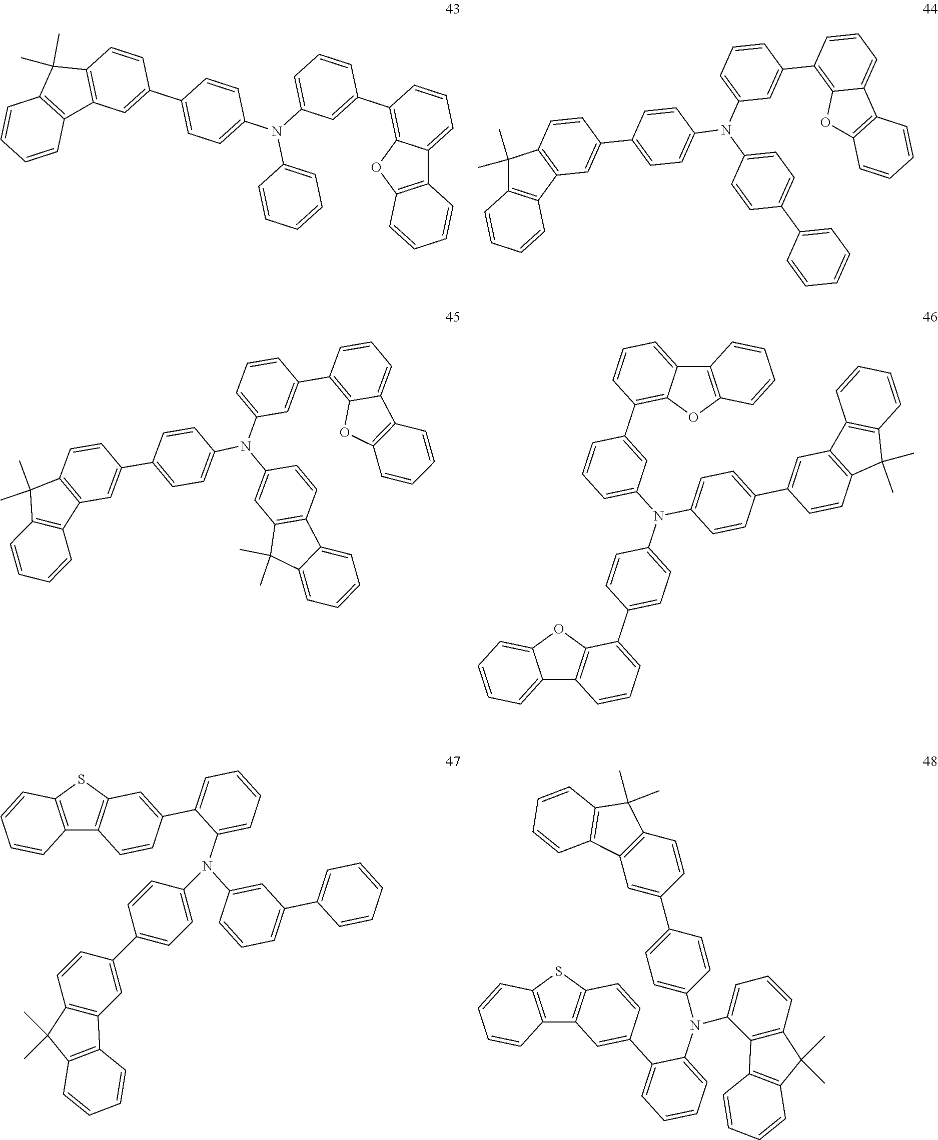

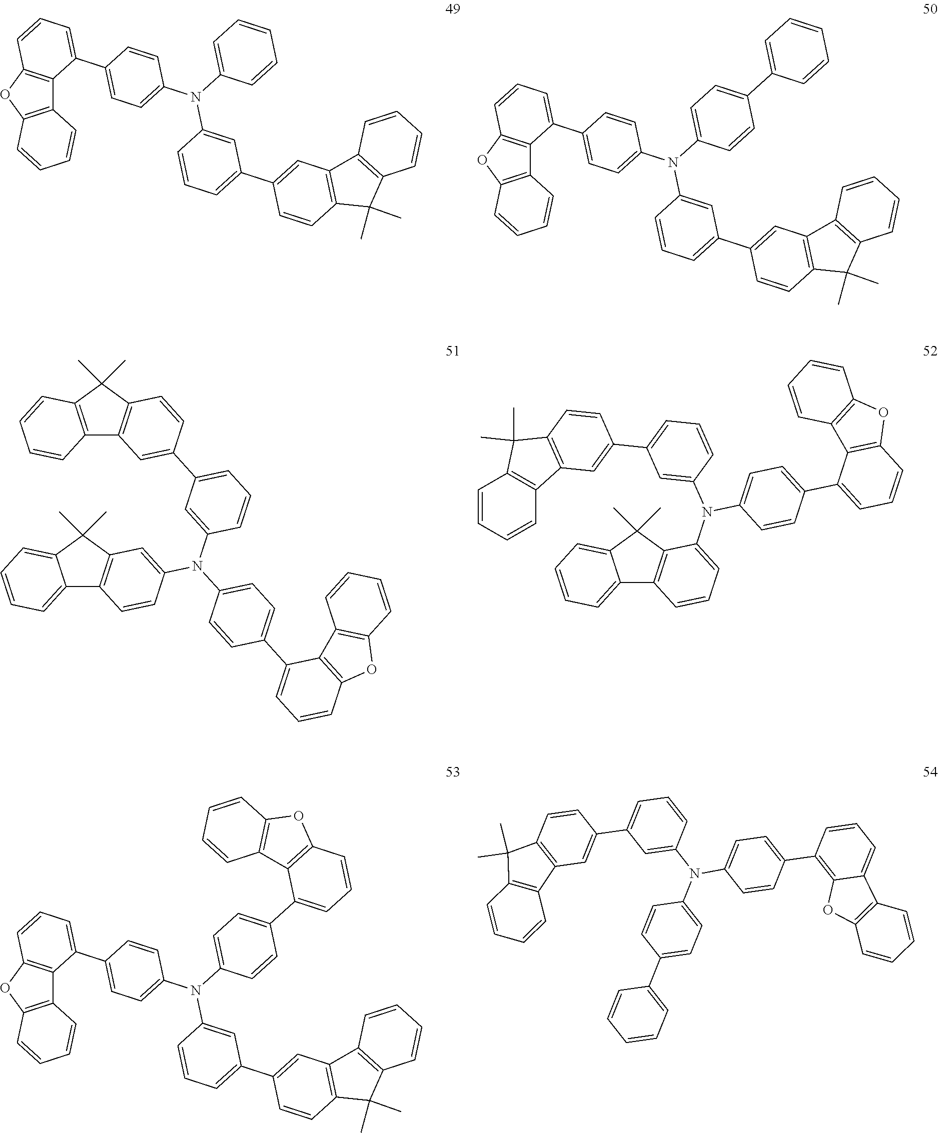

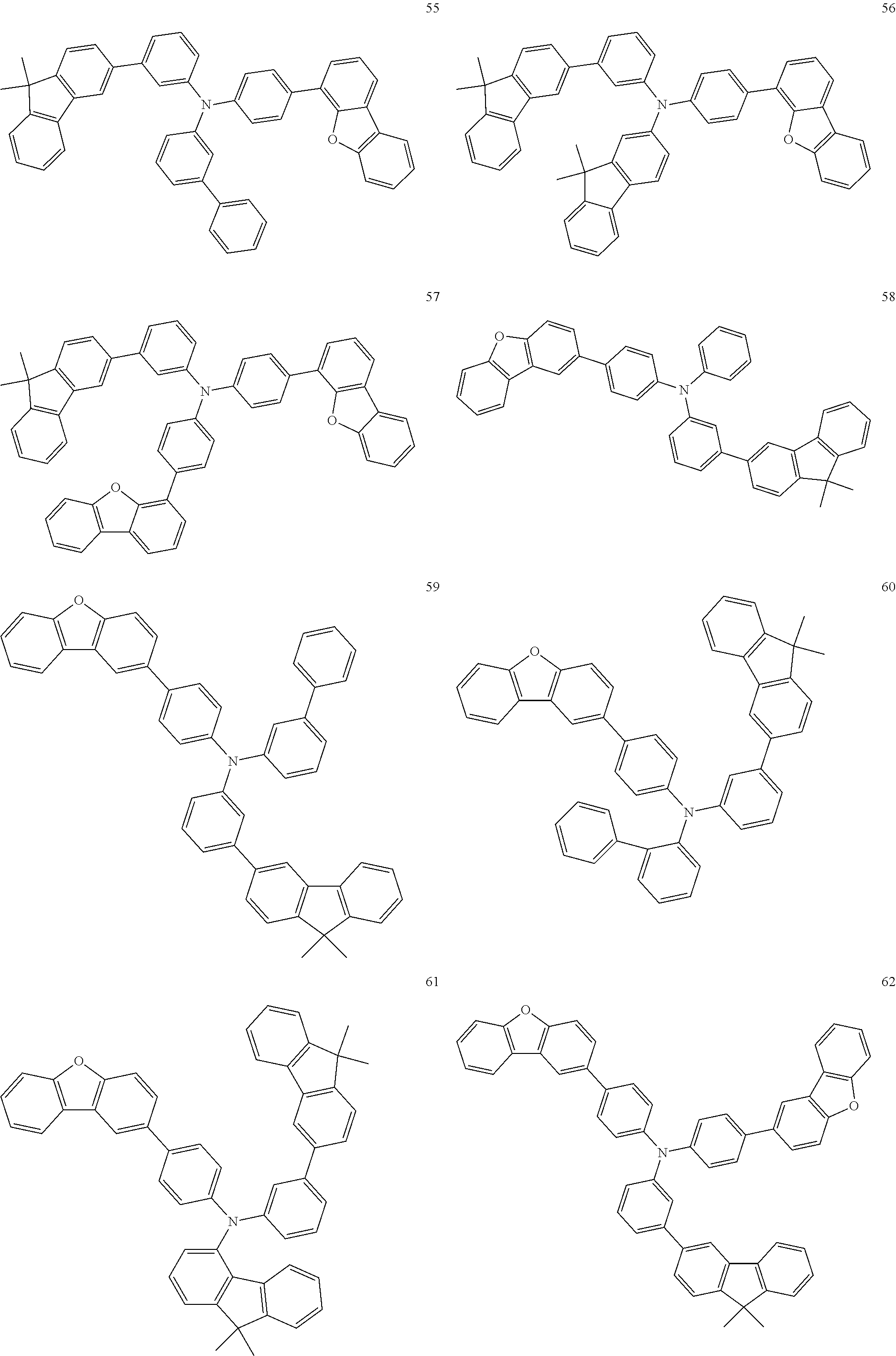

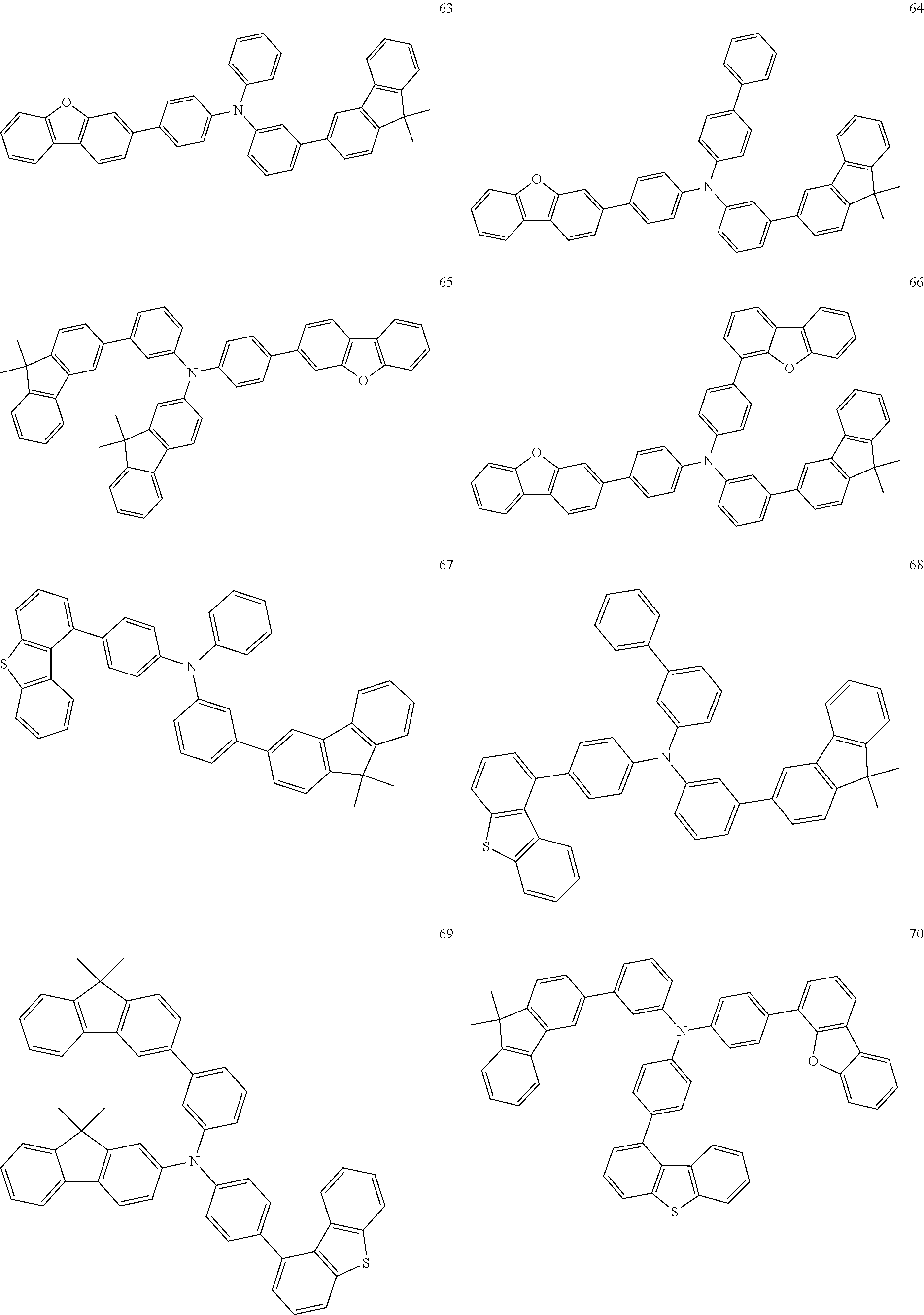

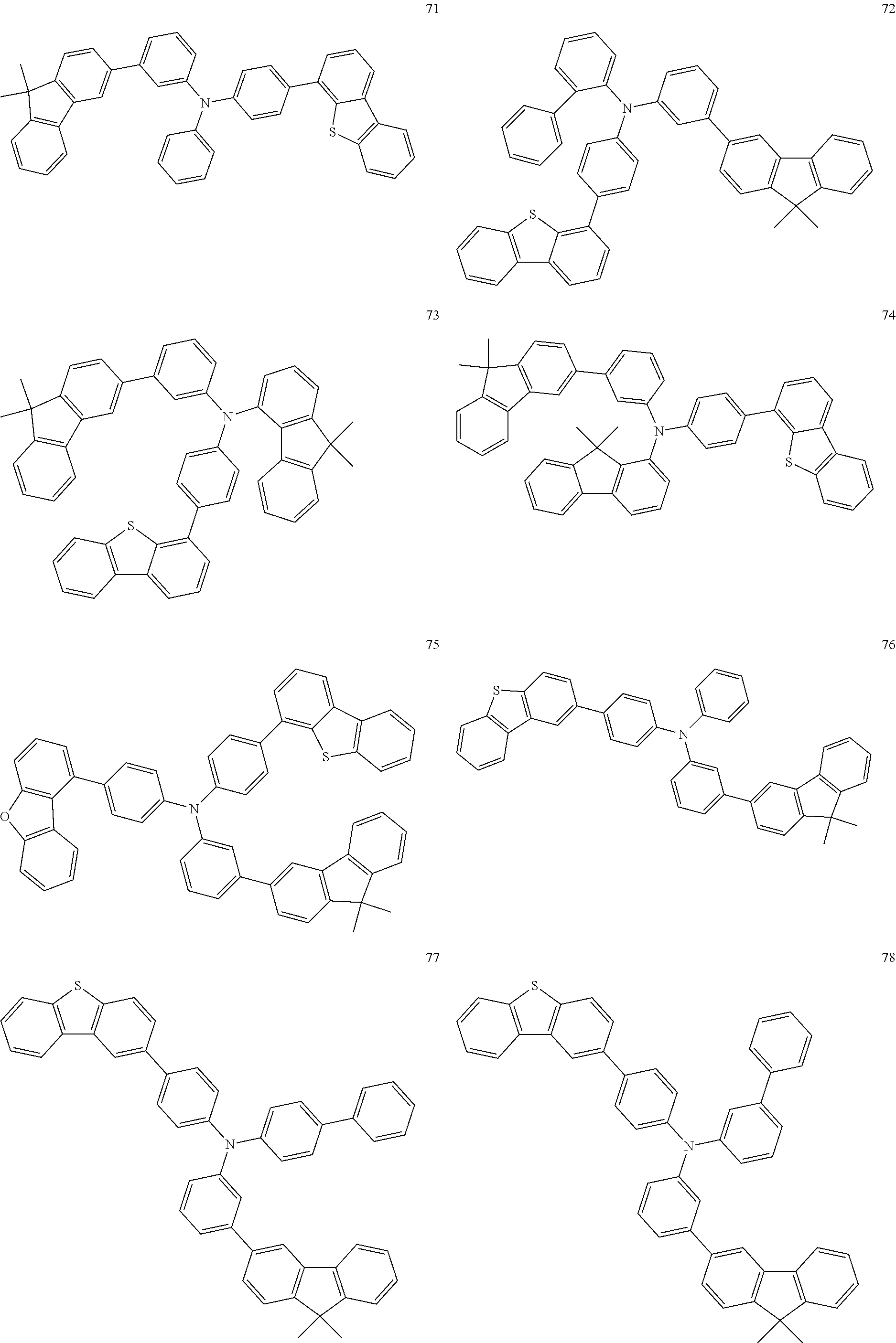

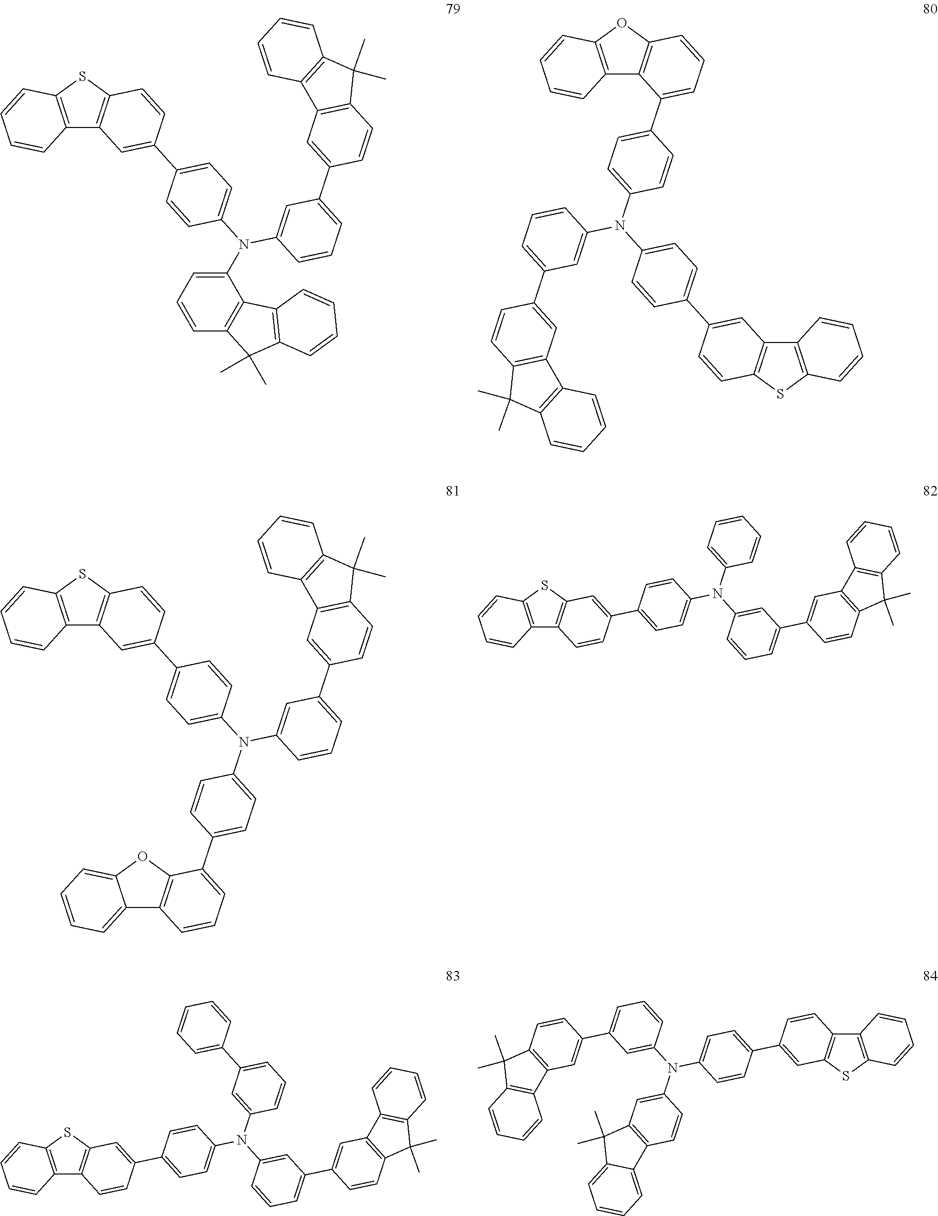

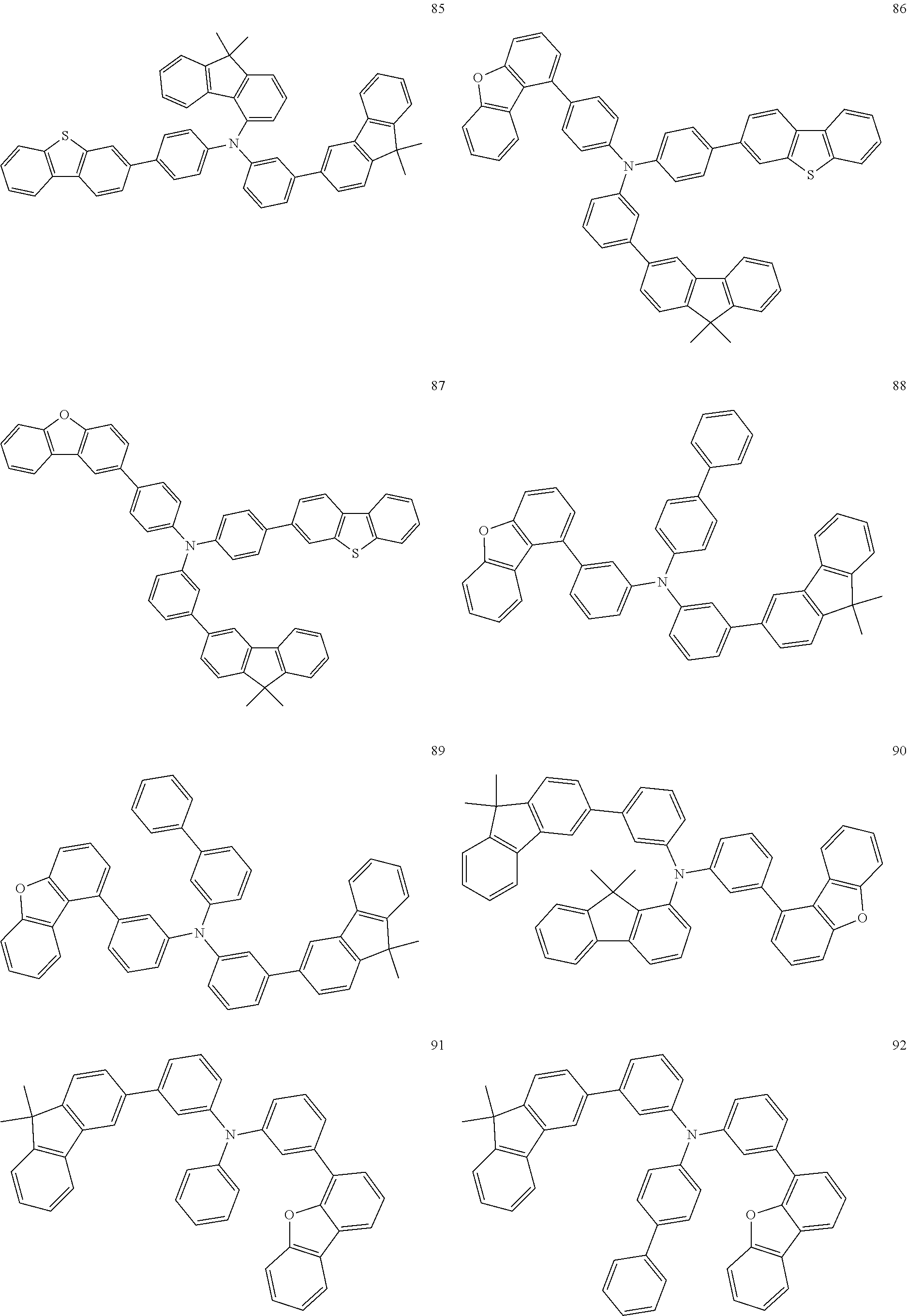

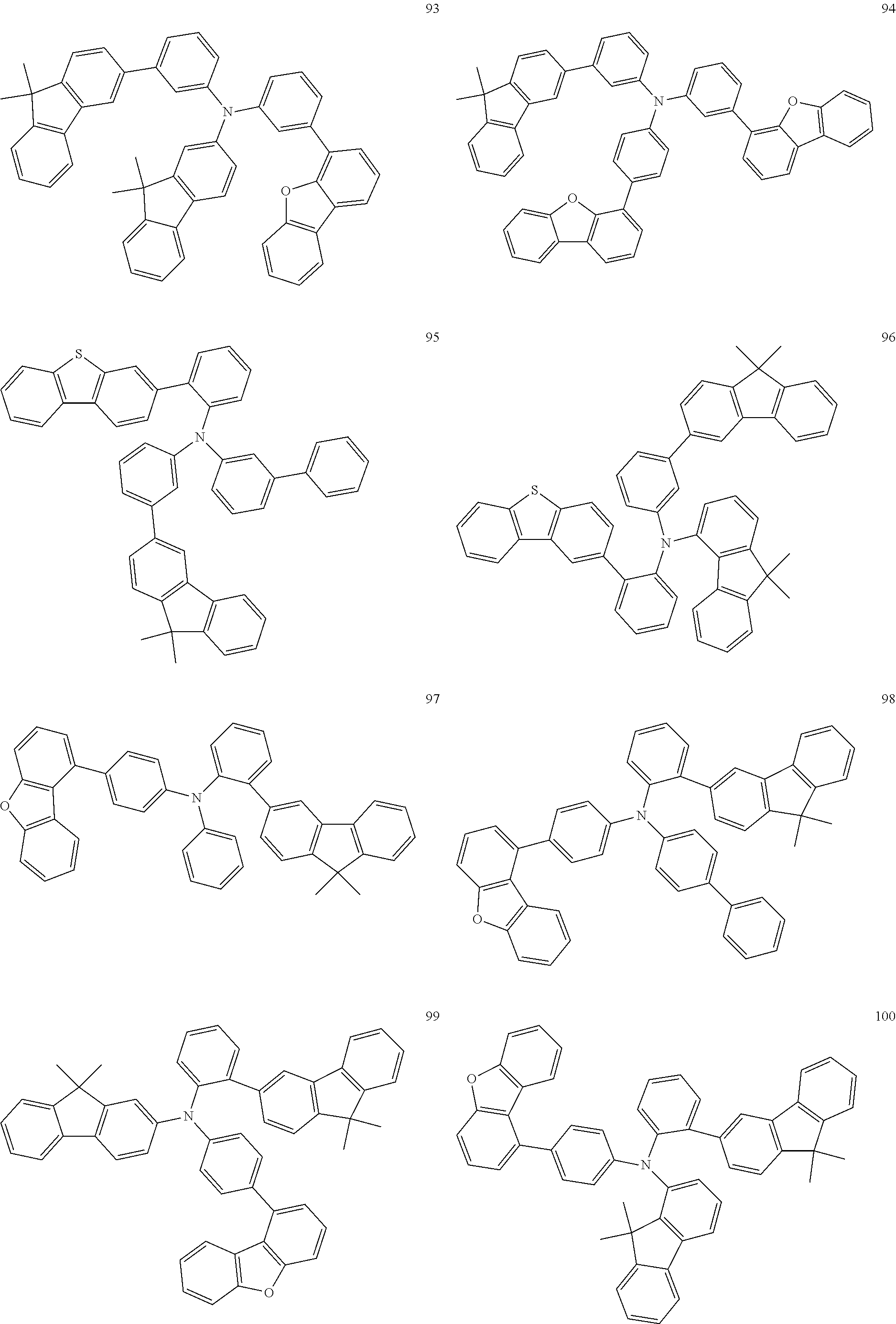

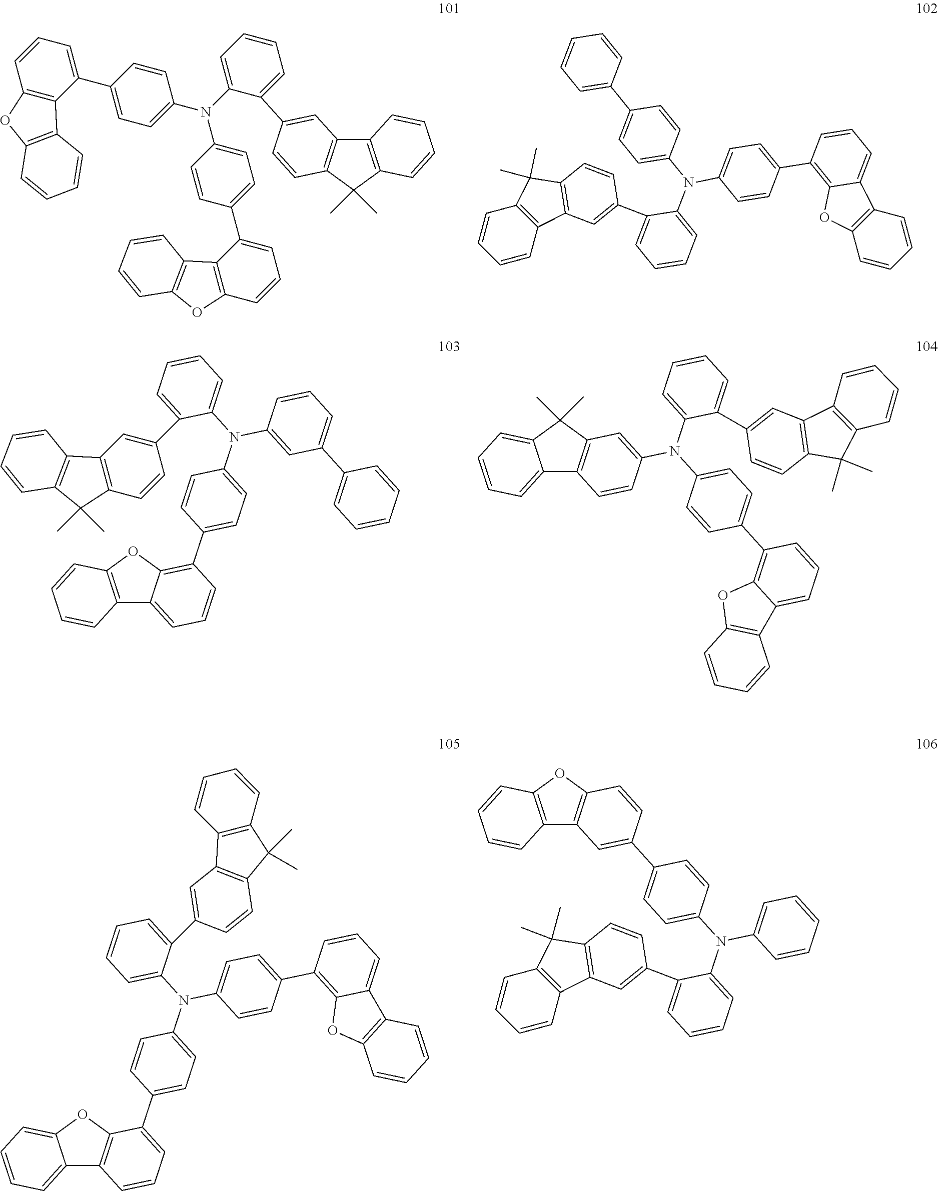

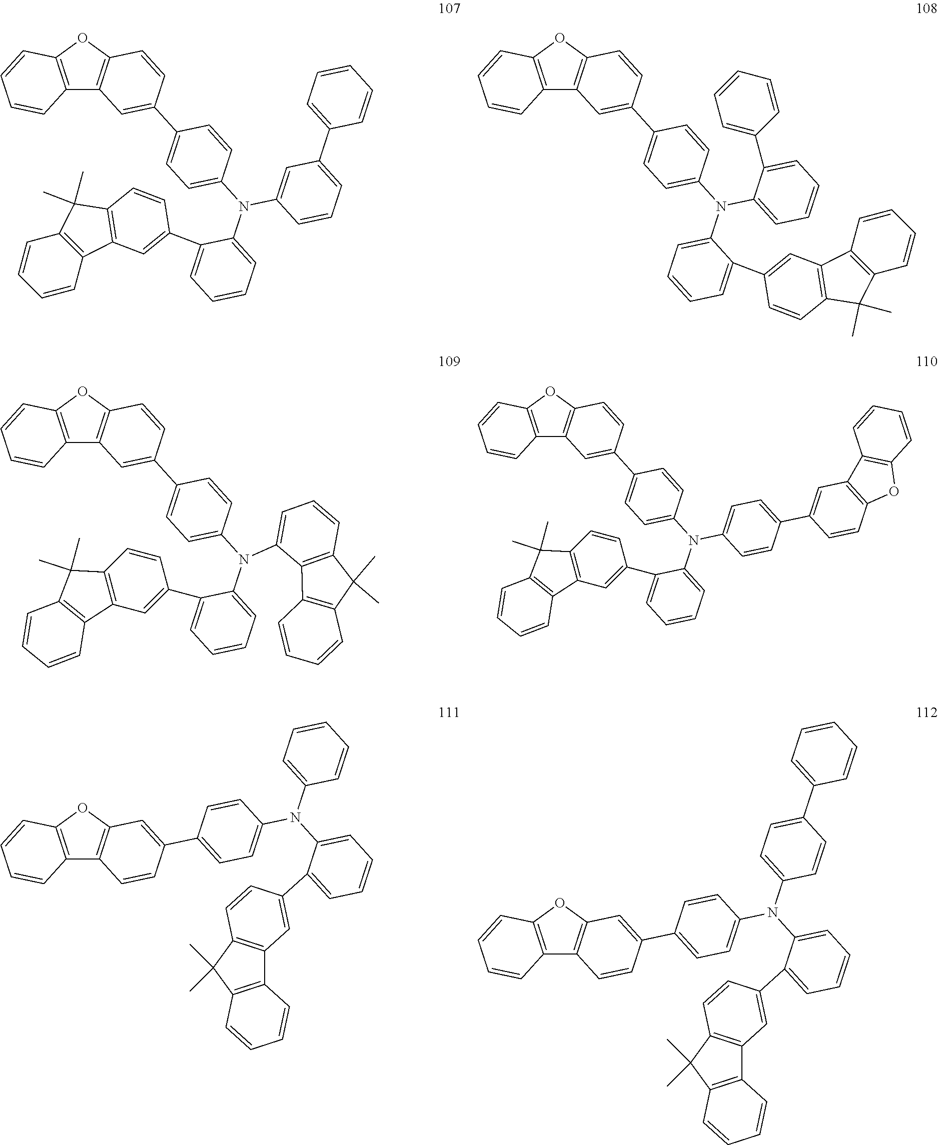

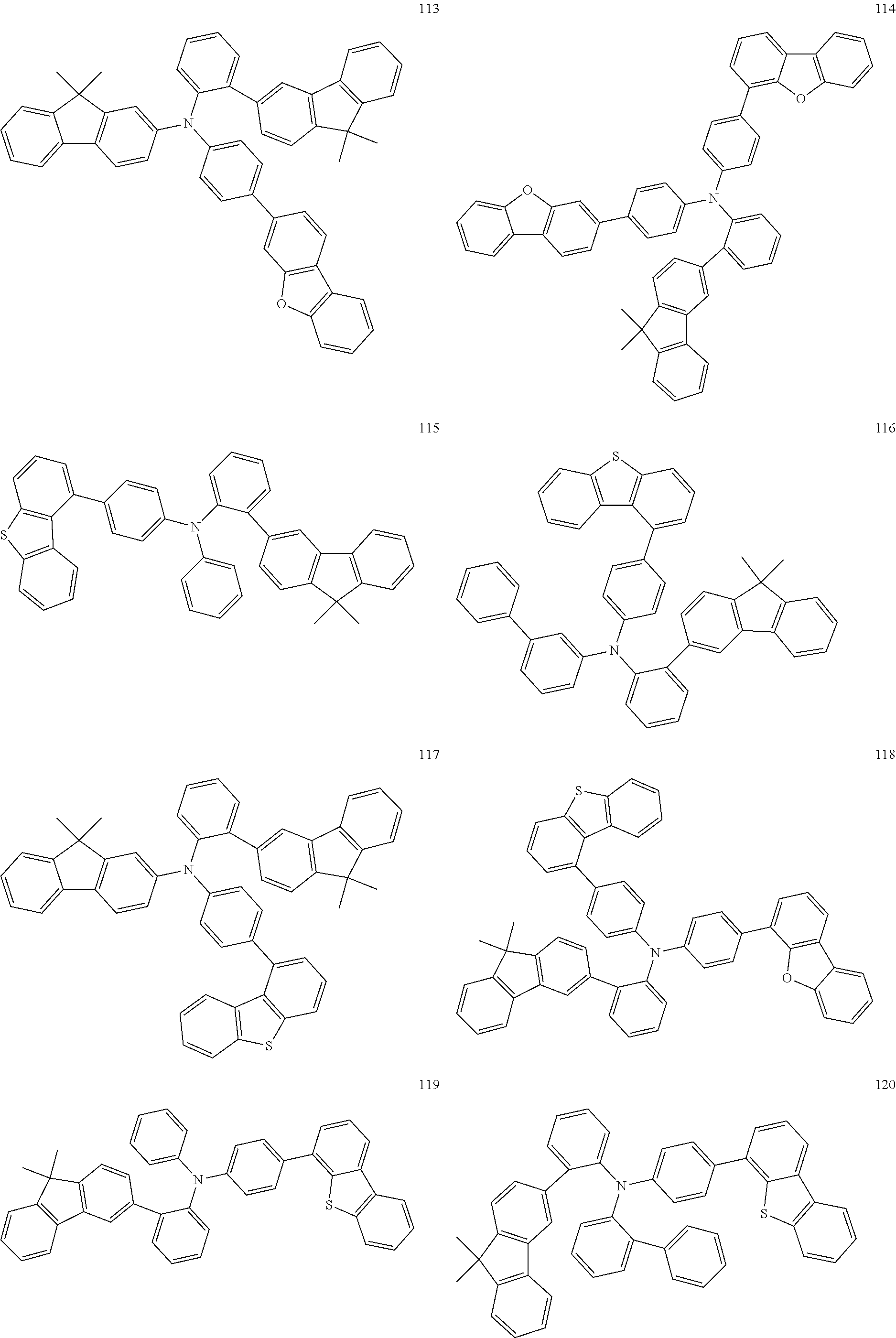

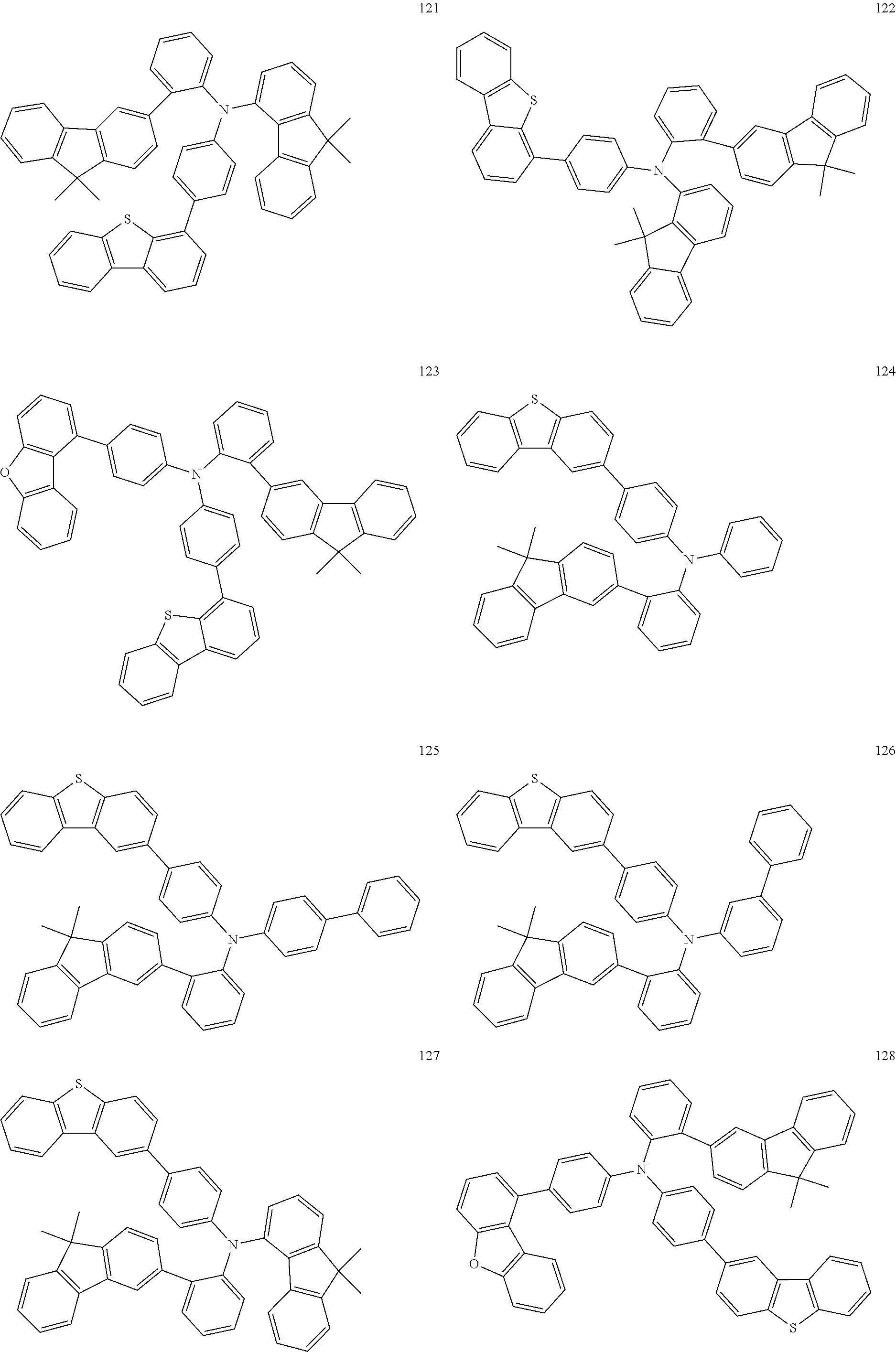

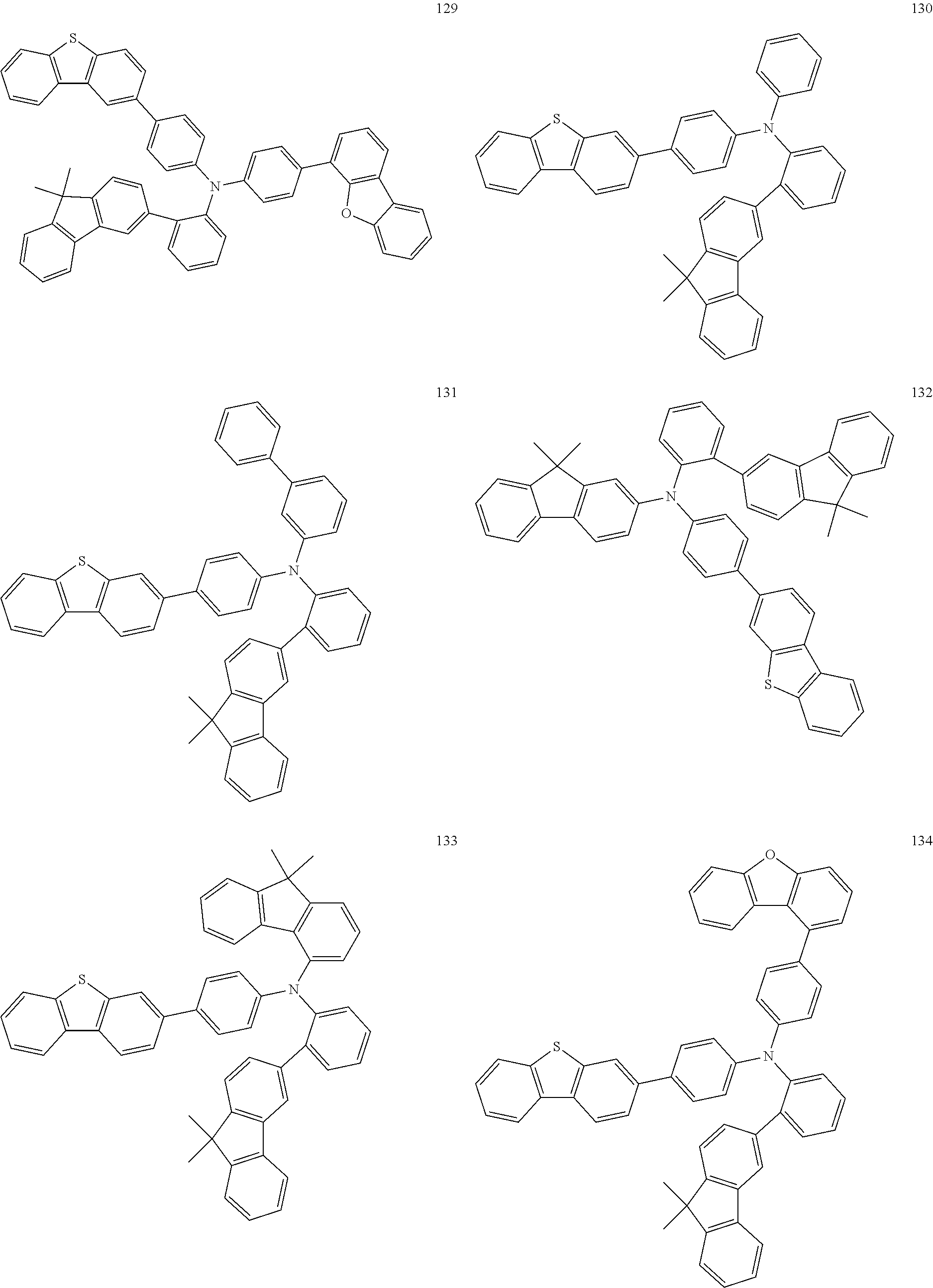

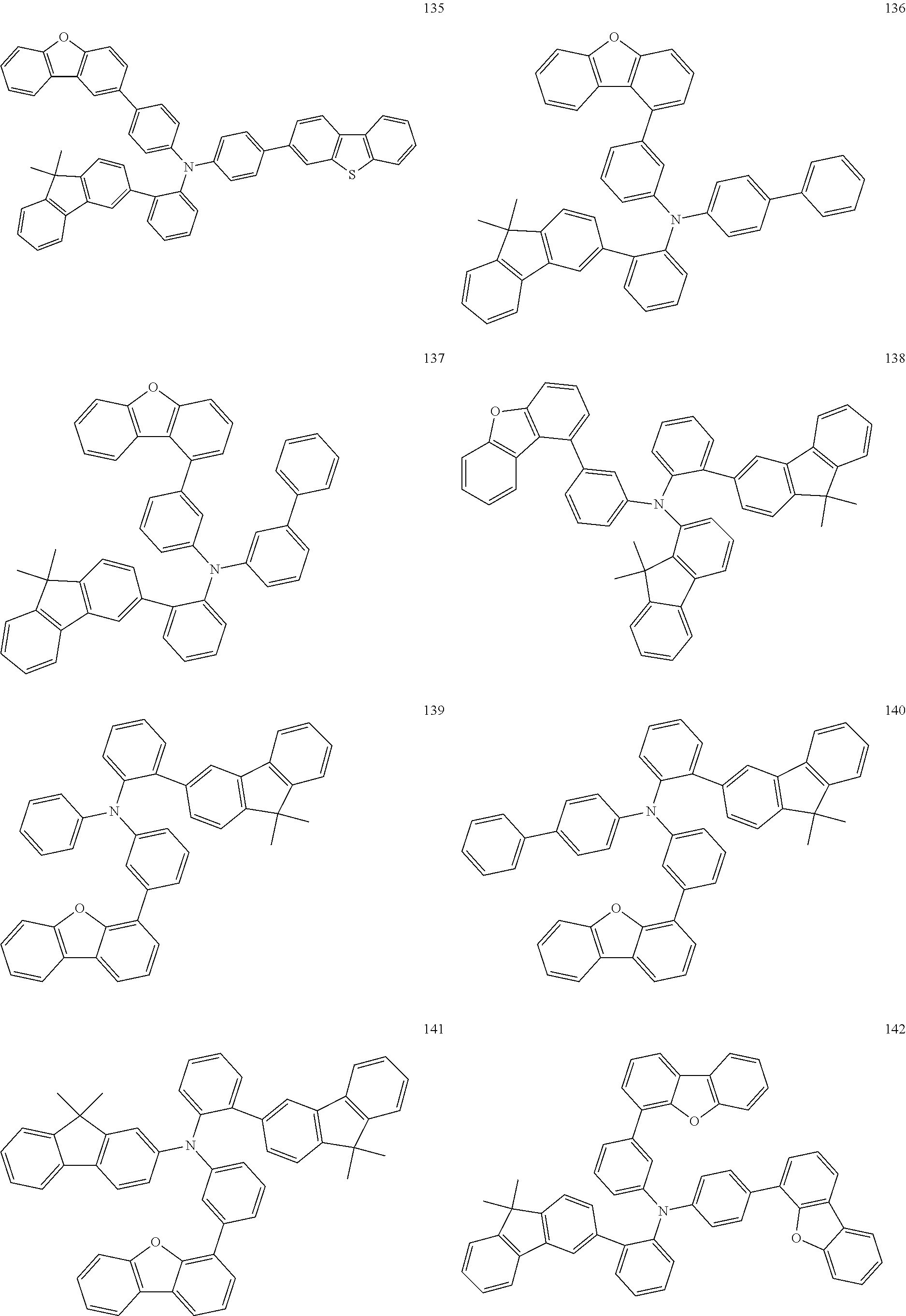

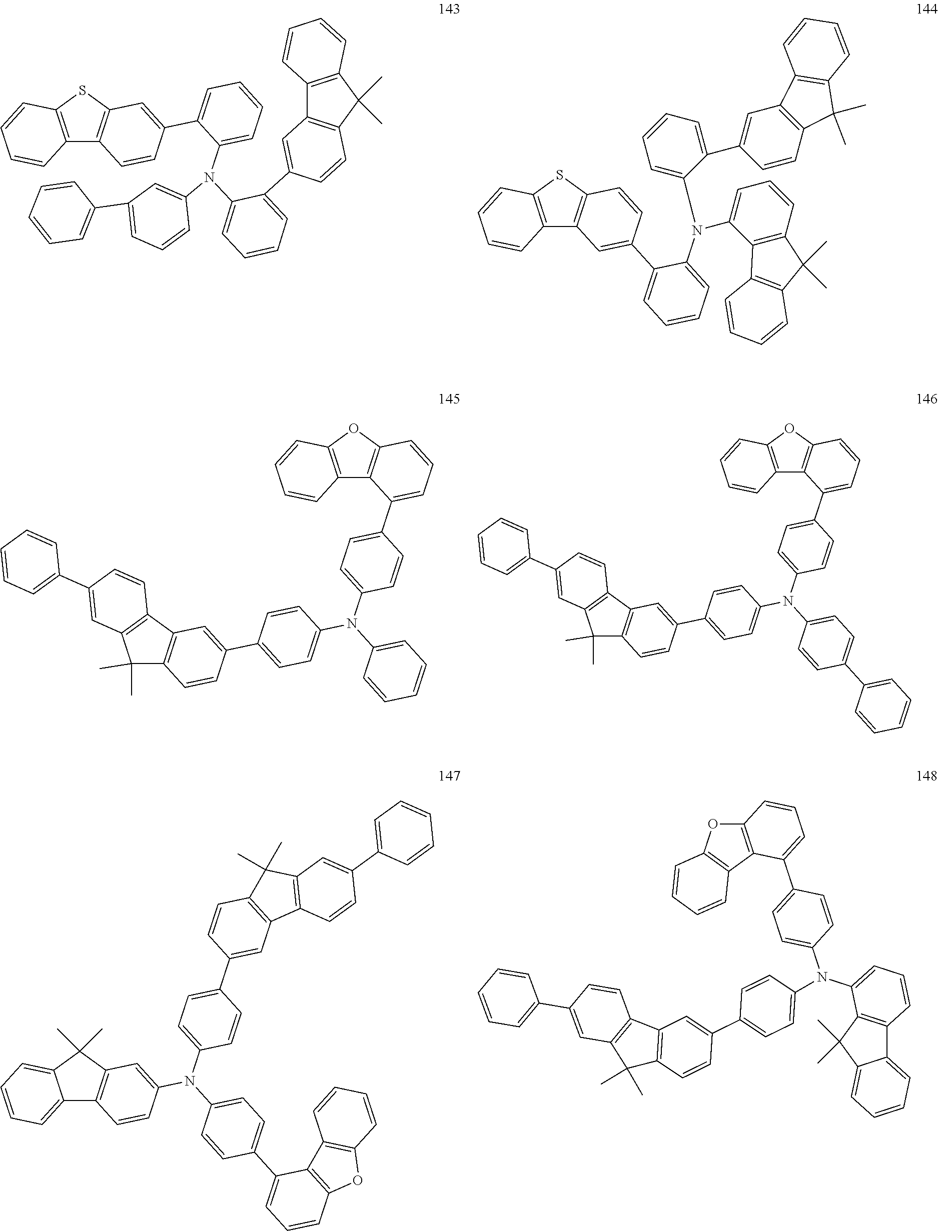

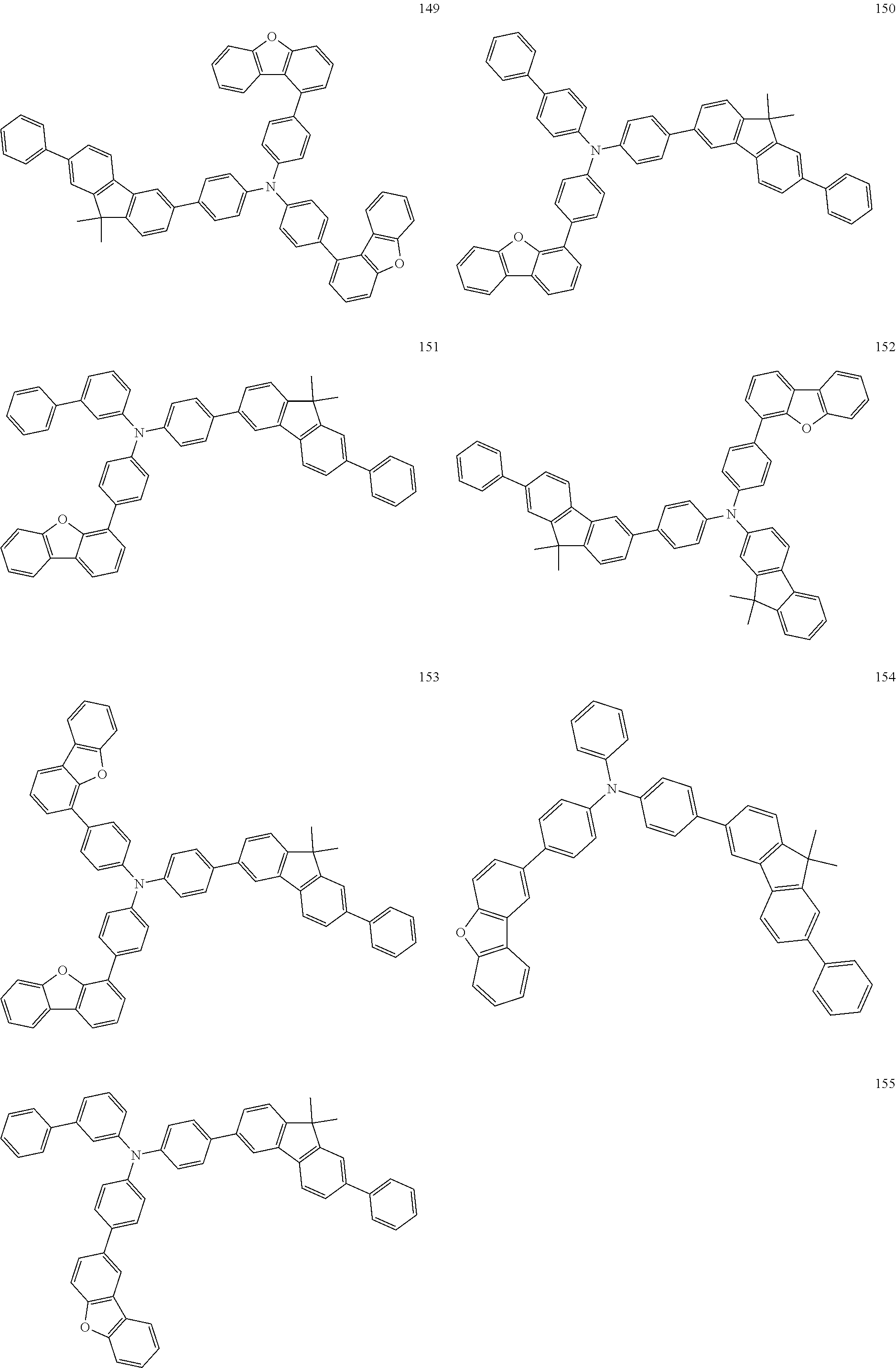

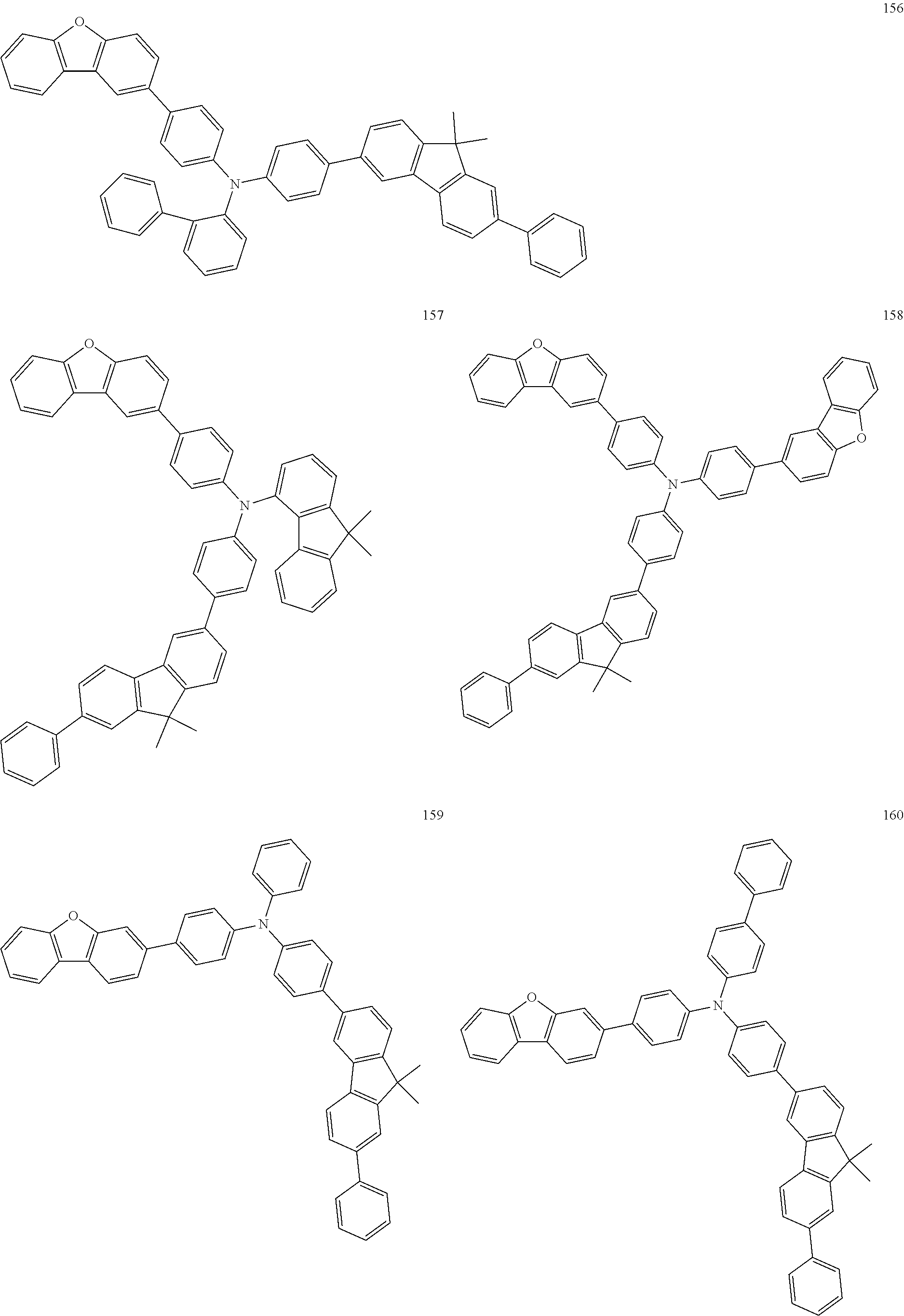

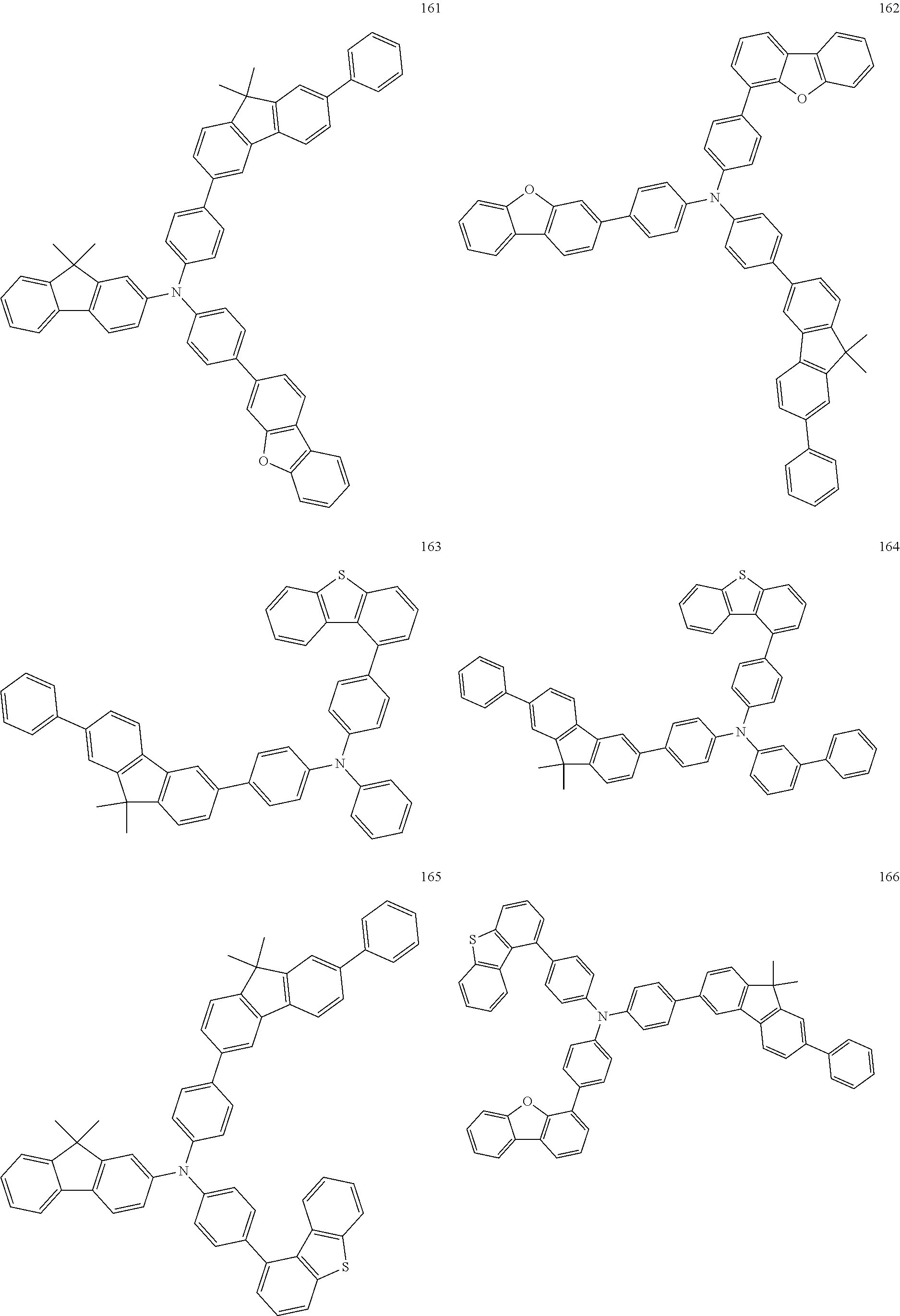

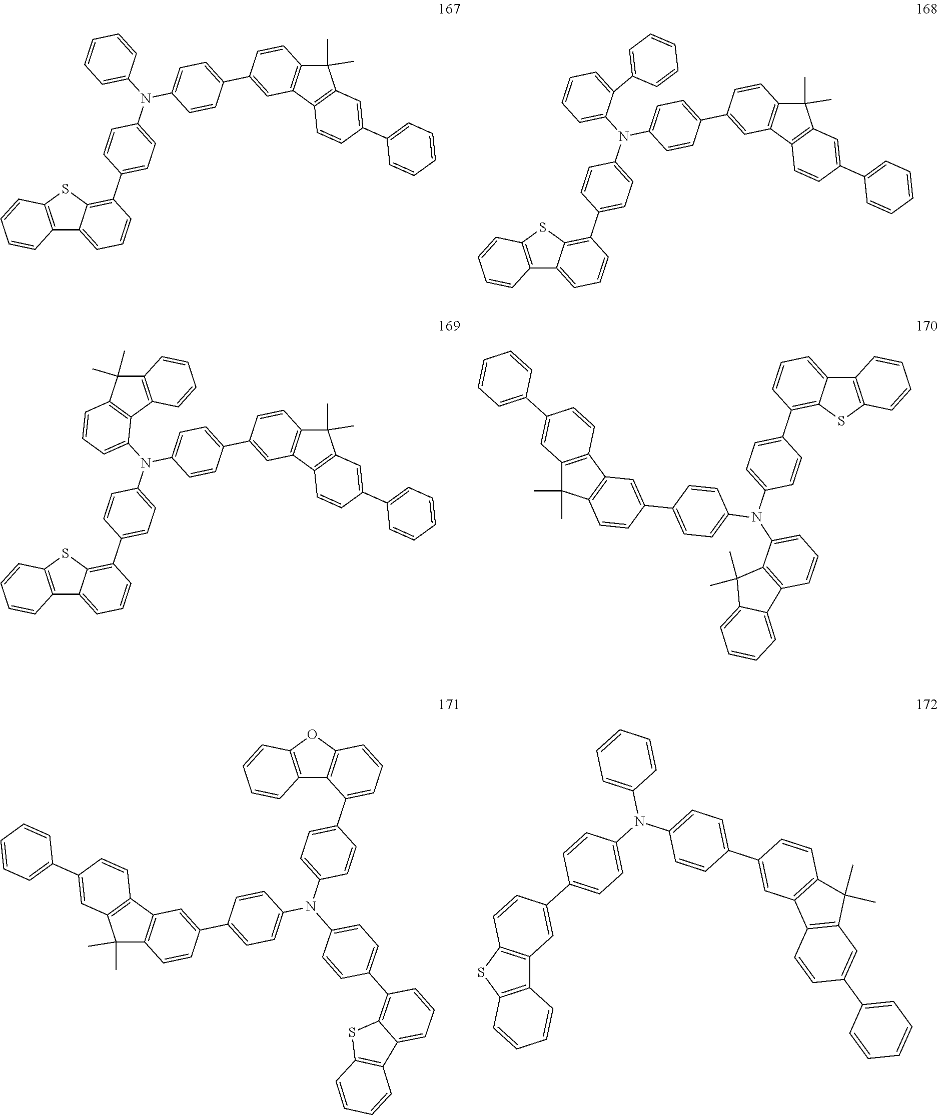

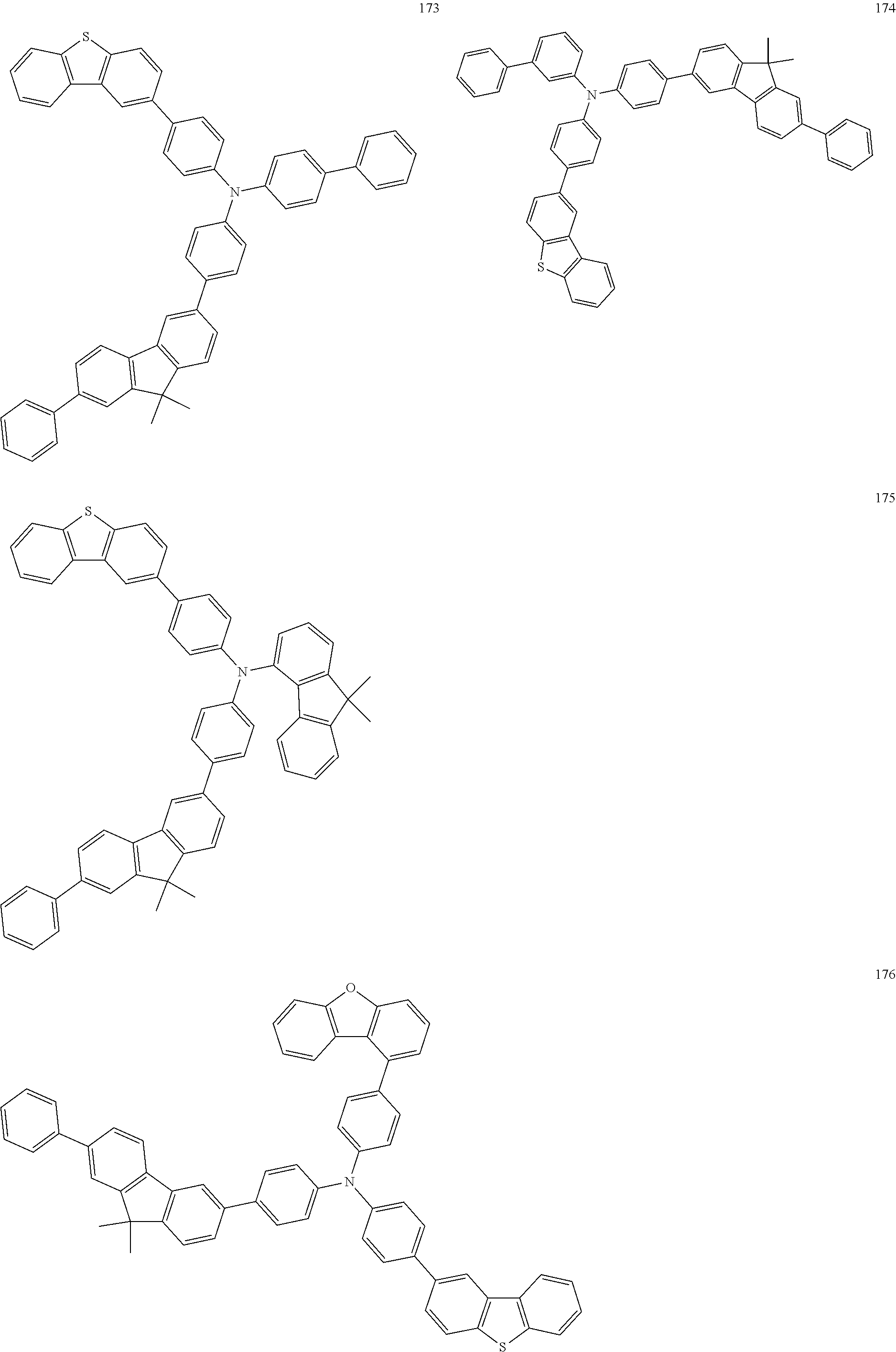 [SMILES: CC1(C)c2ccc(-c3ccc(N(c4ccc(-c5ccccc5)cc4)c4ccc(-c5ccc6sc7ccccc7c6c5)cc4)cc3)cc2-c2ccc(-c3ccccc3)cc21 ; CC1(C)c2ccc(-c3ccc(N(c4ccc(-c5ccc6sc7ccccc7c6c5)cc4)c4cccc(-c5ccccc5)c4)cc3)cc2-c2ccc(-c3ccccc3)cc21 ; CC1(C)c2ccc(-c3ccc(N(c4ccc(-c5ccc6sc7ccccc7c6c5)cc4)c4cccc5c4-c4ccccc4C5(C)C)cc3)cc2-c2ccc(-c3ccccc3)cc21 ; CC1(C)c2ccc(-c3ccc(N(c4ccc(-c5ccc6sc7ccccc7c6c5)cc4)c4ccc(-c5cccc6oc7ccccc7c56)cc4)cc3)cc2-c2ccc(-c3ccccc3)cc21]

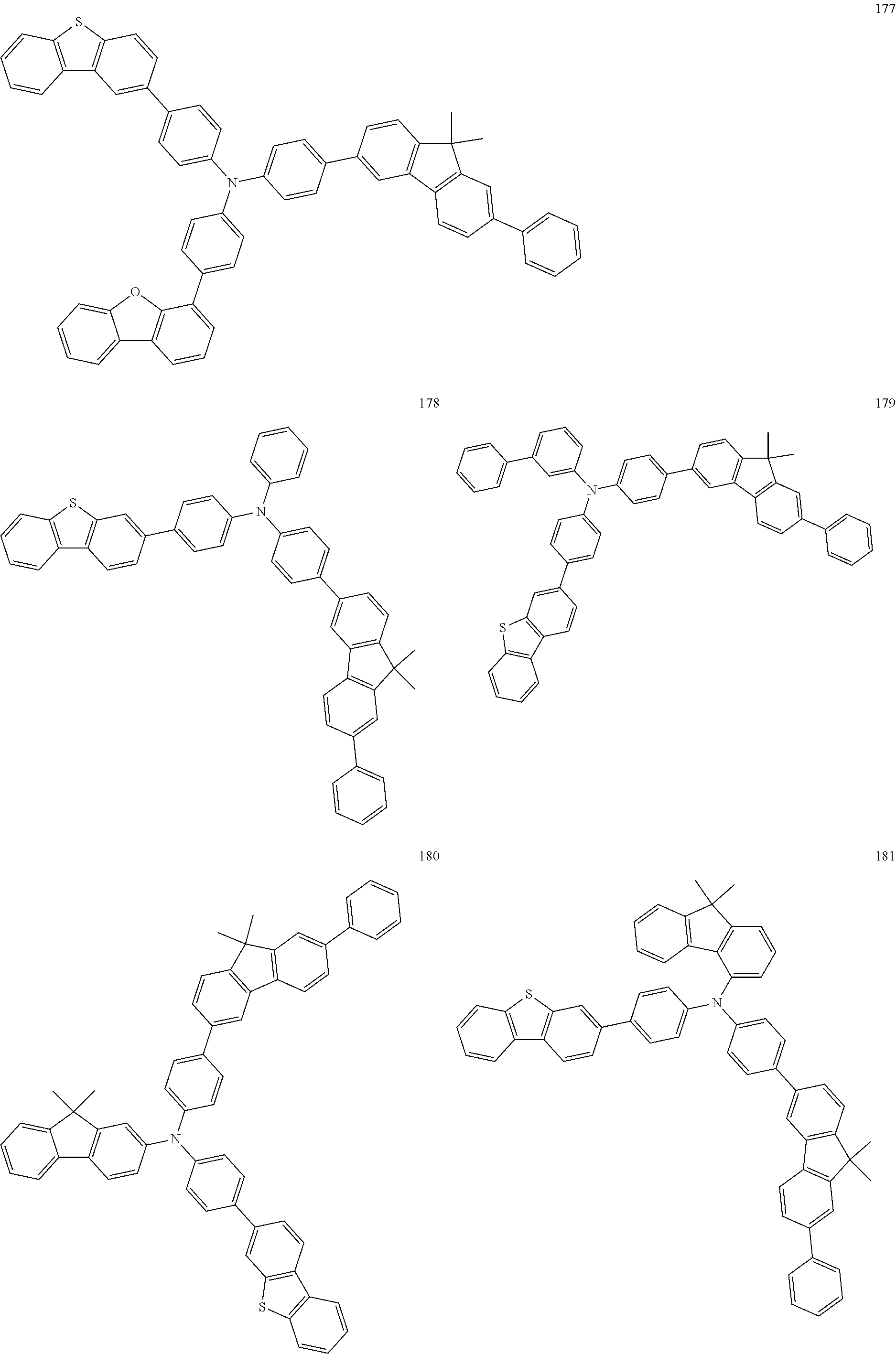

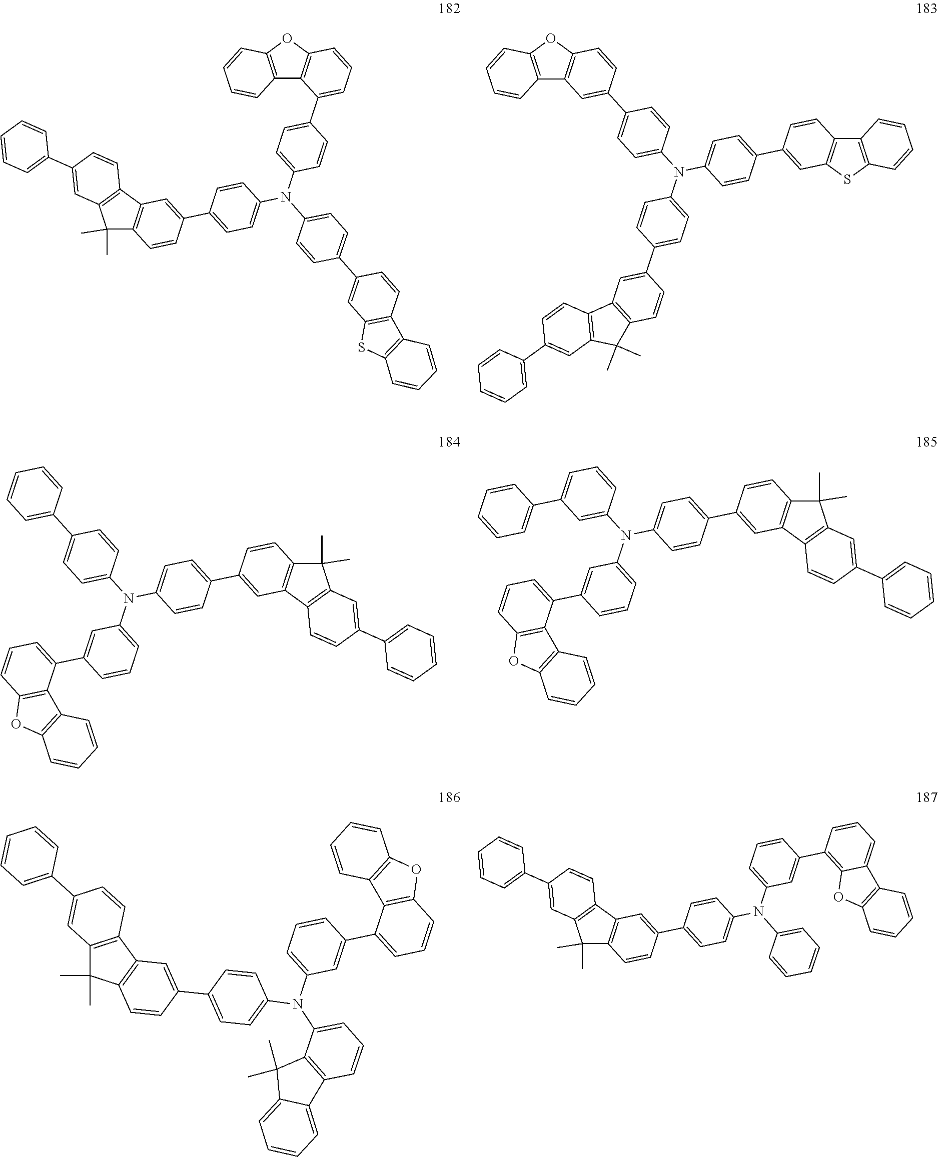

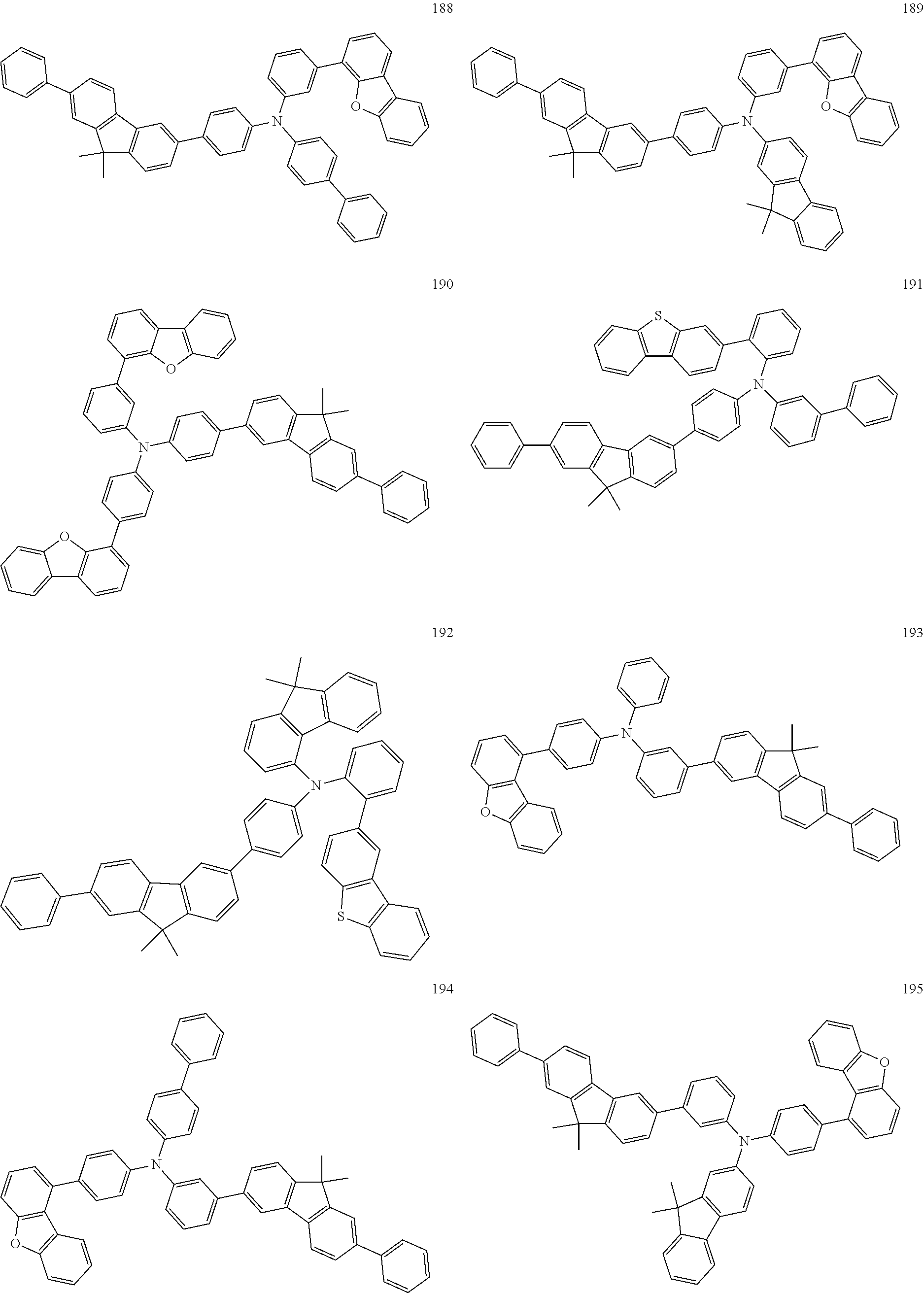

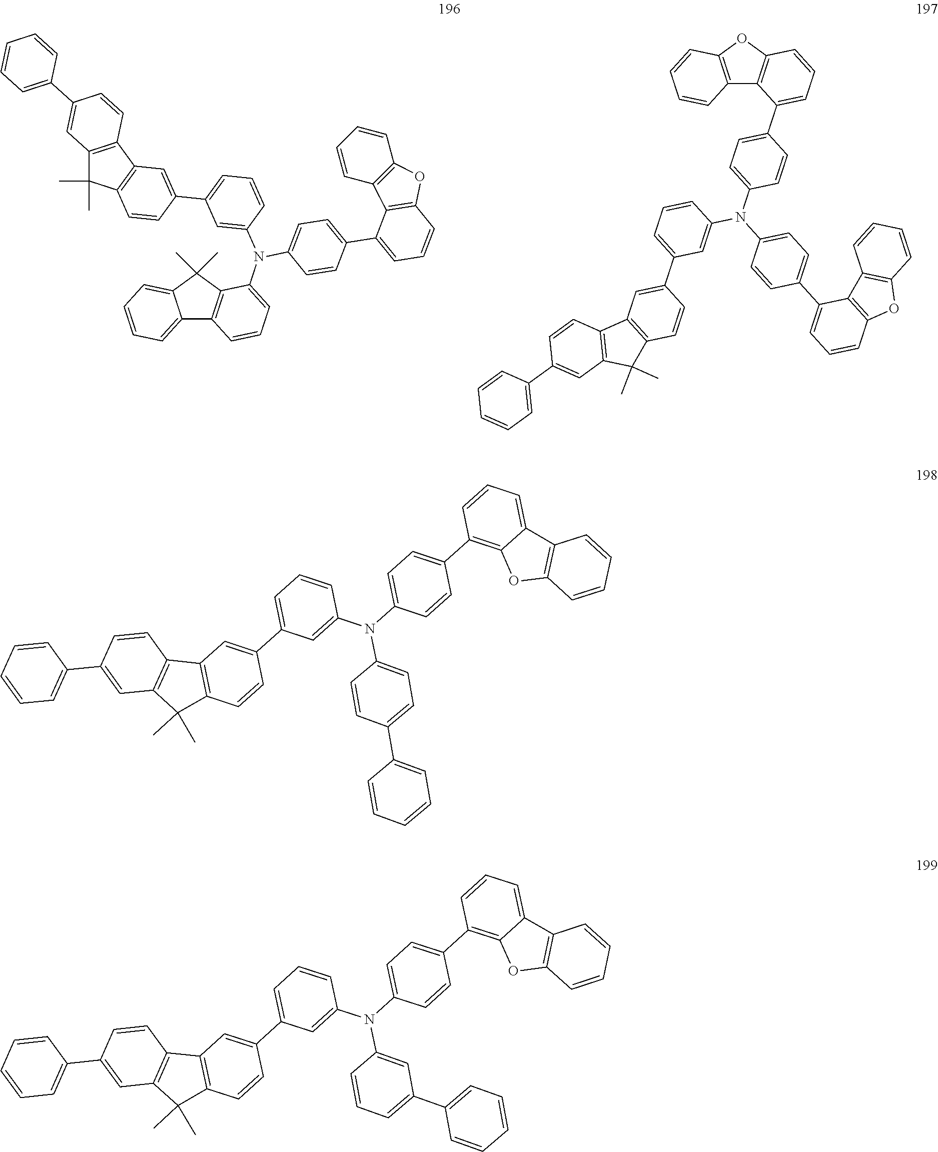 [SMILES: CC1(C)c2ccc(-c3cccc(N(c4ccc(-c5cccc6oc7ccccc7c56)cc4)c4cccc5c4C(C)(C)c4ccccc4-5)c3)cc2-c2ccc(-c3ccccc3)cc21 ; CC1(C)c2ccc(-c3cccc(N(c4ccc(-c5cccc6oc7ccccc7c56)cc4)c4ccc(-c5cccc6oc7ccccc7c56)cc4)c3)cc2-c2ccc(-c3ccccc3)cc21 ; CC1(C)c2ccc(-c3cccc(N(c4ccc(-c5ccccc5)cc4)c4ccc(-c5cccc6c5oc5ccccc56)cc4)c3)cc2-c2ccc(-c3ccccc3)cc21 ; CC1(C)c2ccc(-c3cccc(N(c4ccc(-c5cccc6c5oc5ccccc56)cc4)c4cccc(-c5ccccc5)c4)c3)cc2-c2ccc(-c3ccccc3)cc21]

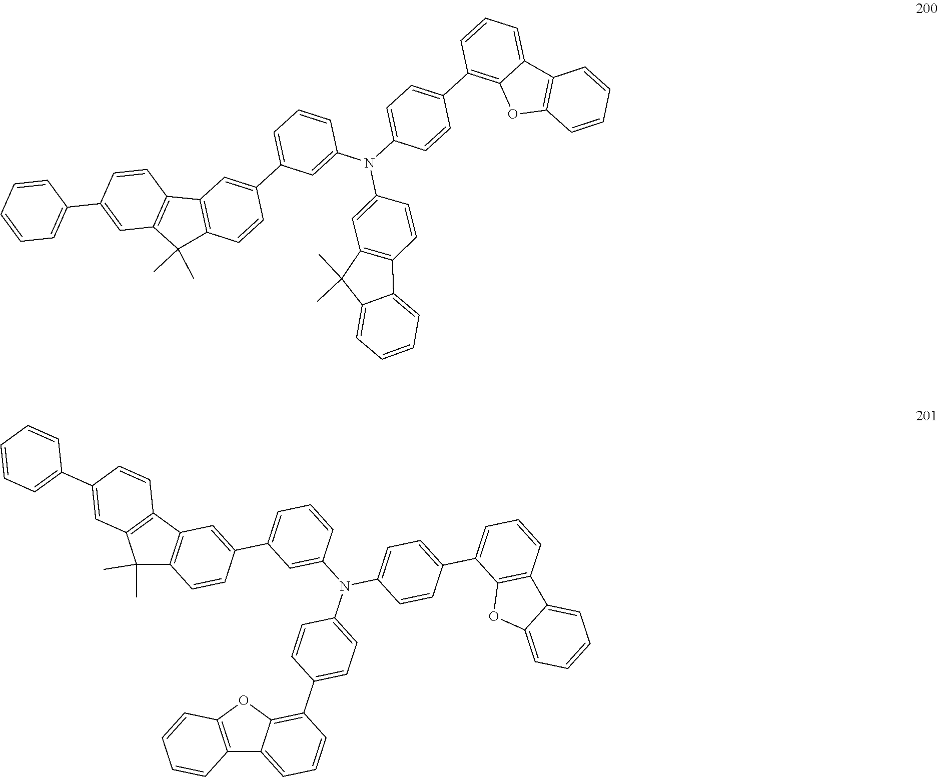 [SMILES: CC1(C)c2ccc(-c3cccc(N(c4ccc(-c5cccc6c5oc5ccccc56)cc4)c4ccc5c(c4)C(C)(C)c4ccccc4-5)c3)cc2-c2ccc(-c3ccccc3)cc21 ; CC1(C)c2ccc(-c3cccc(N(c4ccc(-c5cccc6c5oc5ccccc56)cc4)c4ccc(-c5cccc6c5oc5ccccc56)cc4)c3)cc2-c2ccc(-c3ccccc3)cc21]

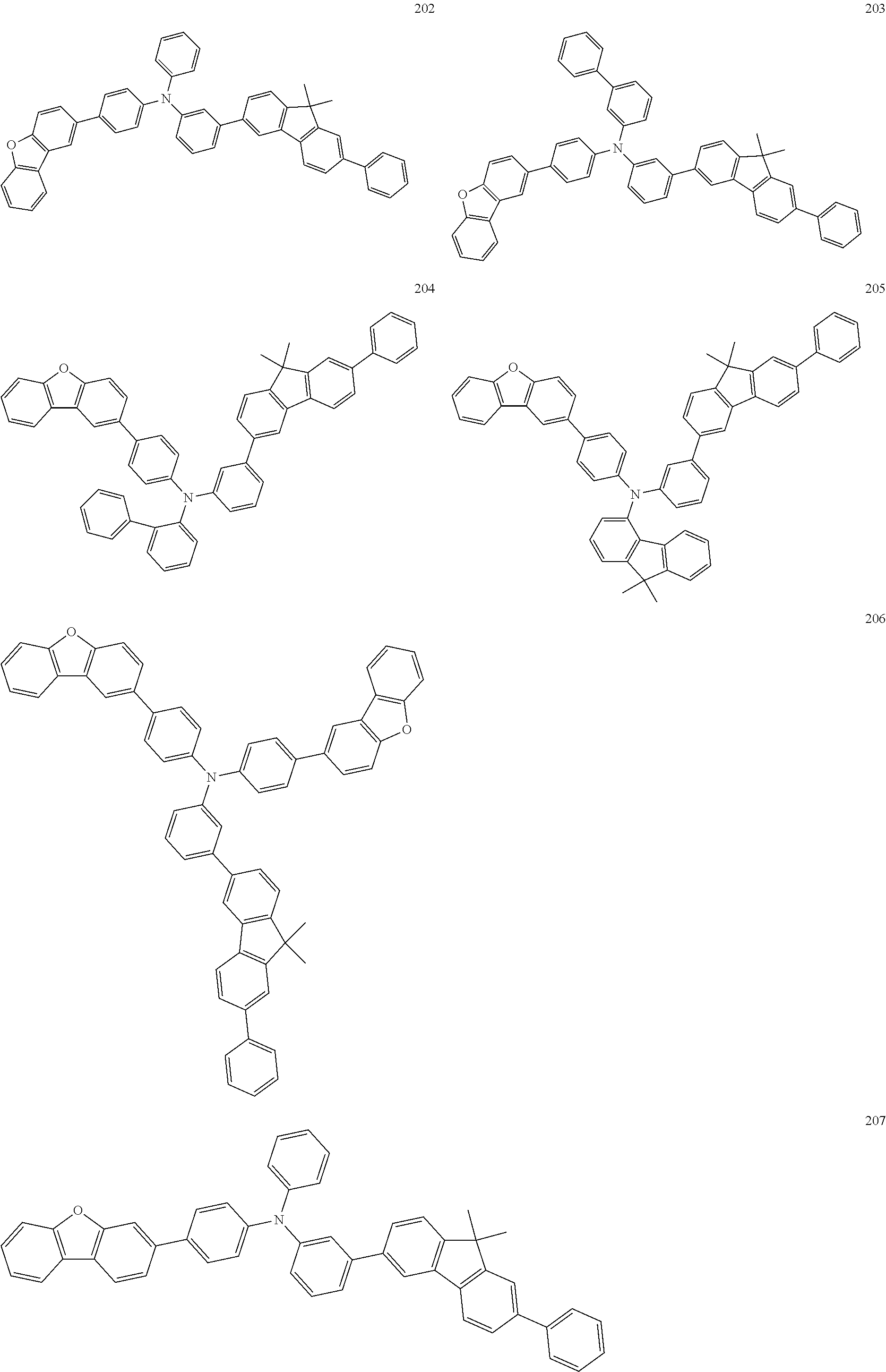

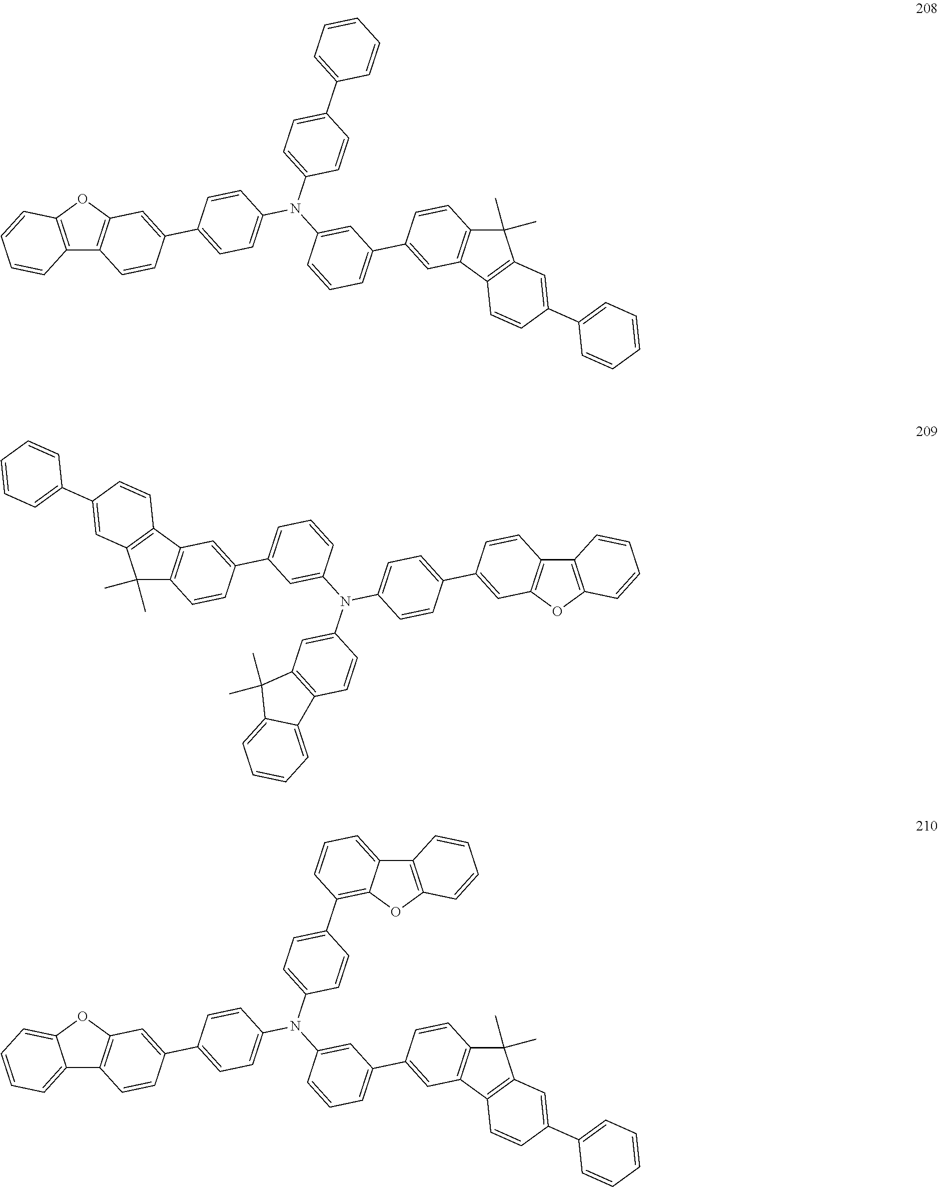 [SMILES: CC1(C)c2ccc(-c3cccc(N(c4ccc(-c5ccccc5)cc4)c4ccc(-c5ccc6c(c5)oc5ccccc56)cc4)c3)cc2-c2ccc(-c3ccccc3)cc21 ; CC1(C)c2ccc(-c3cccc(N(c4ccc(-c5ccc6c(c5)oc5ccccc56)cc4)c4ccc5c(c4)C(C)(C)c4ccccc4-5)c3)cc2-c2ccc(-c3ccccc3)cc21 ; CC1(C)c2ccc(-c3cccc(N(c4ccc(-c5ccc6c(c5)oc5ccccc56)cc4)c4ccc(-c5cccc6c5oc5ccccc56)cc4)c3)cc2-c2ccc(-c3ccccc3)cc21]

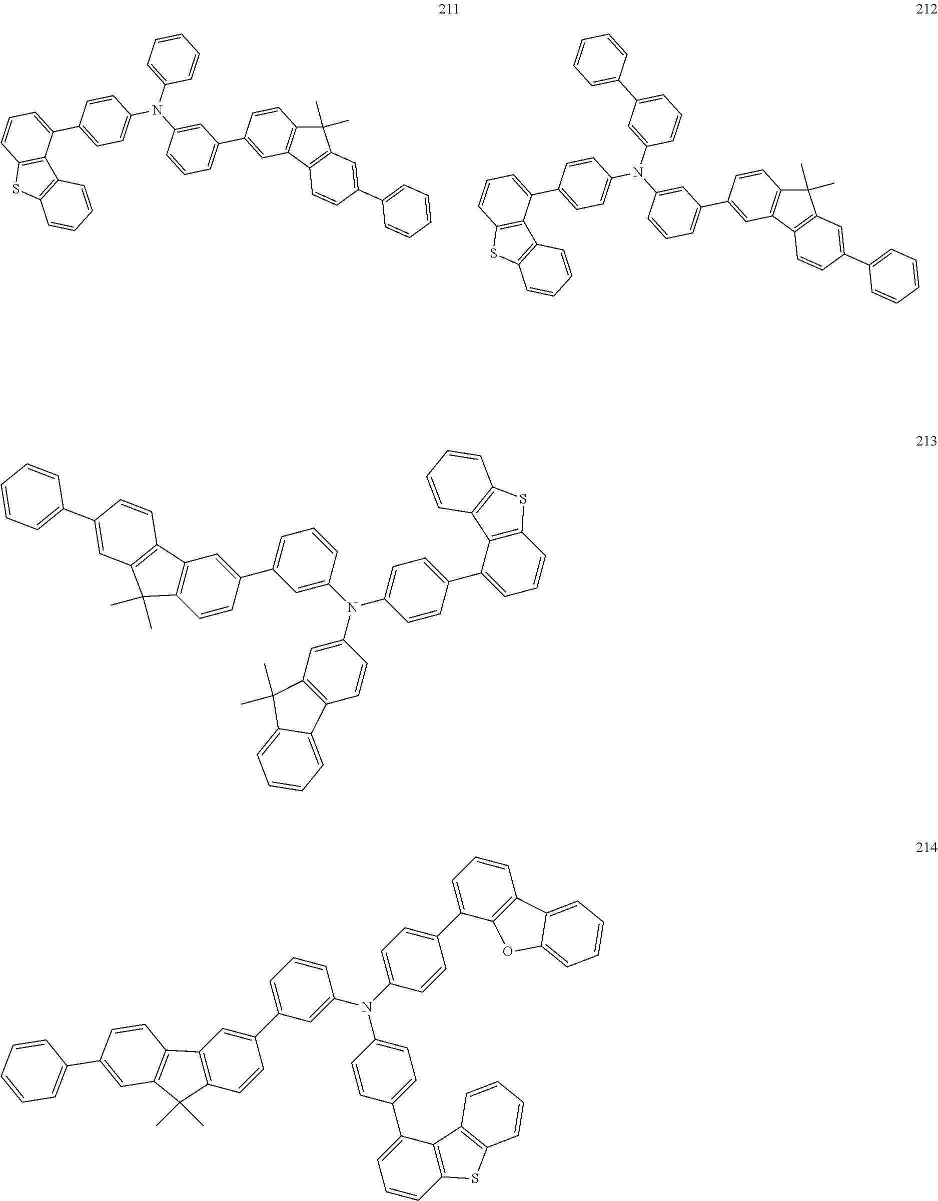 [SMILES: CC1(C)c2ccc(-c3cccc(N(c4ccccc4)c4ccc(-c5cccc6sc7ccccc7c56)cc4)c3)cc2-c2ccc(-c3ccccc3)cc21 ; CC1(C)c2ccc(-c3cccc(N(c4ccc(-c5cccc6sc7ccccc7c56)cc4)c4cccc(-c5ccccc5)c4)c3)cc2-c2ccc(-c3ccccc3)cc21 ; CC1(C)c2ccc(-c3cccc(N(c4ccc(-c5cccc6sc7ccccc7c56)cc4)c4ccc5c(c4)C(C)(C)c4ccccc4-5)c3)cc2-c2ccc(-c3ccccc3)cc21 ; CC1(C)c2ccc(-c3cccc(N(c4ccc(-c5cccc6c5oc5ccccc56)cc4)c4ccc(-c5cccc6sc7ccccc7c56)cc4)c3)cc2-c2ccc(-c3ccccc3)cc21]

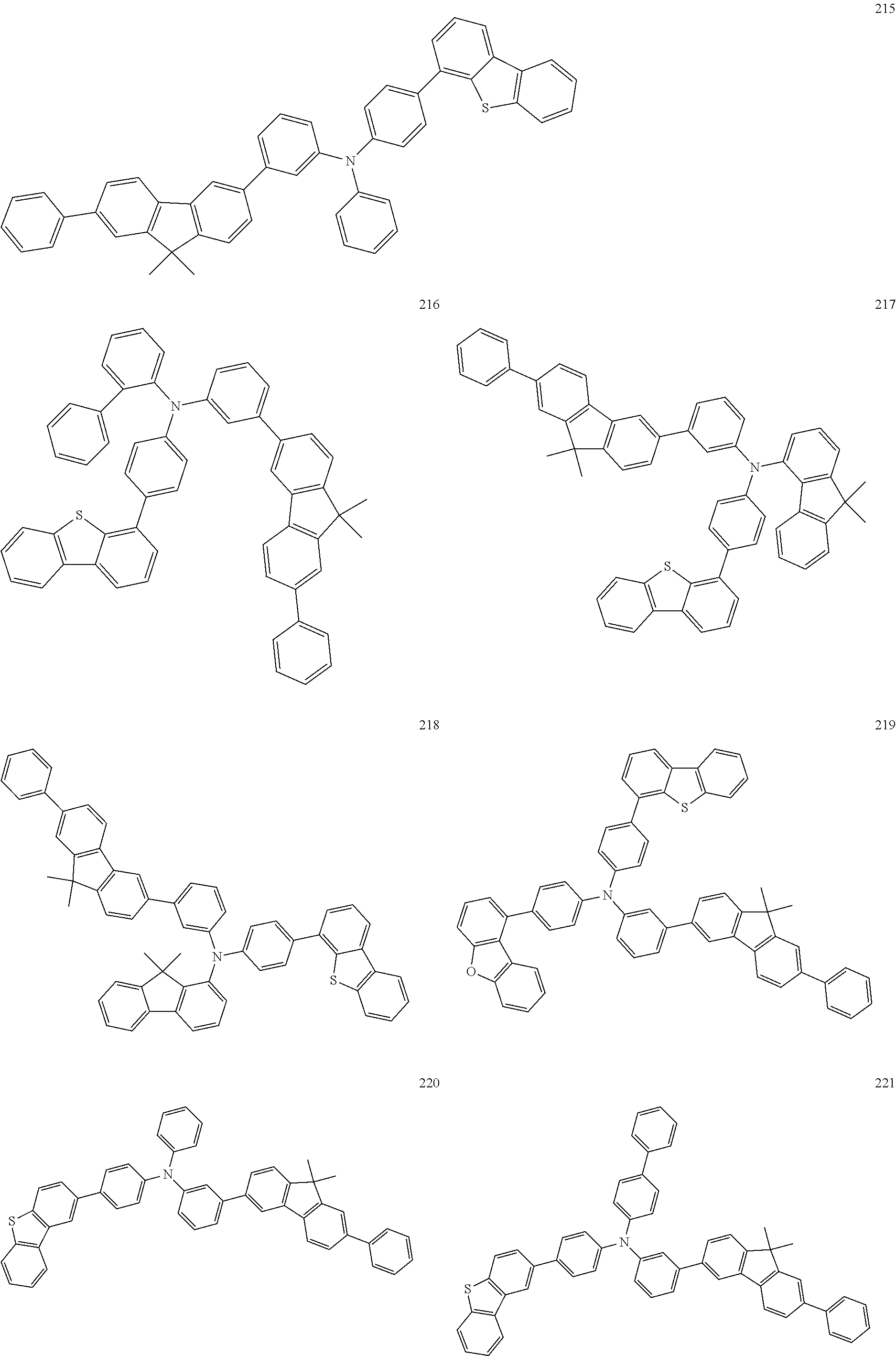

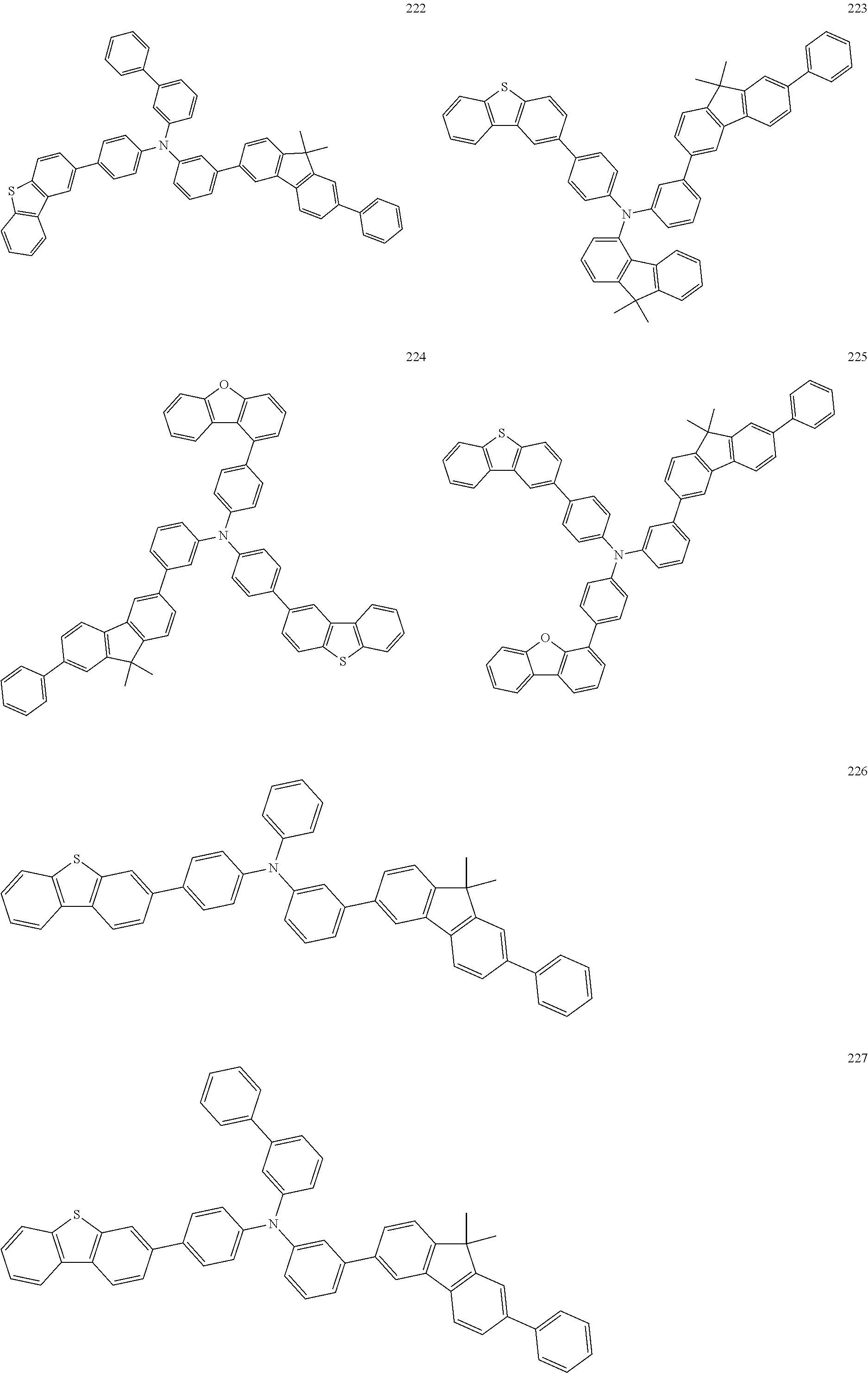

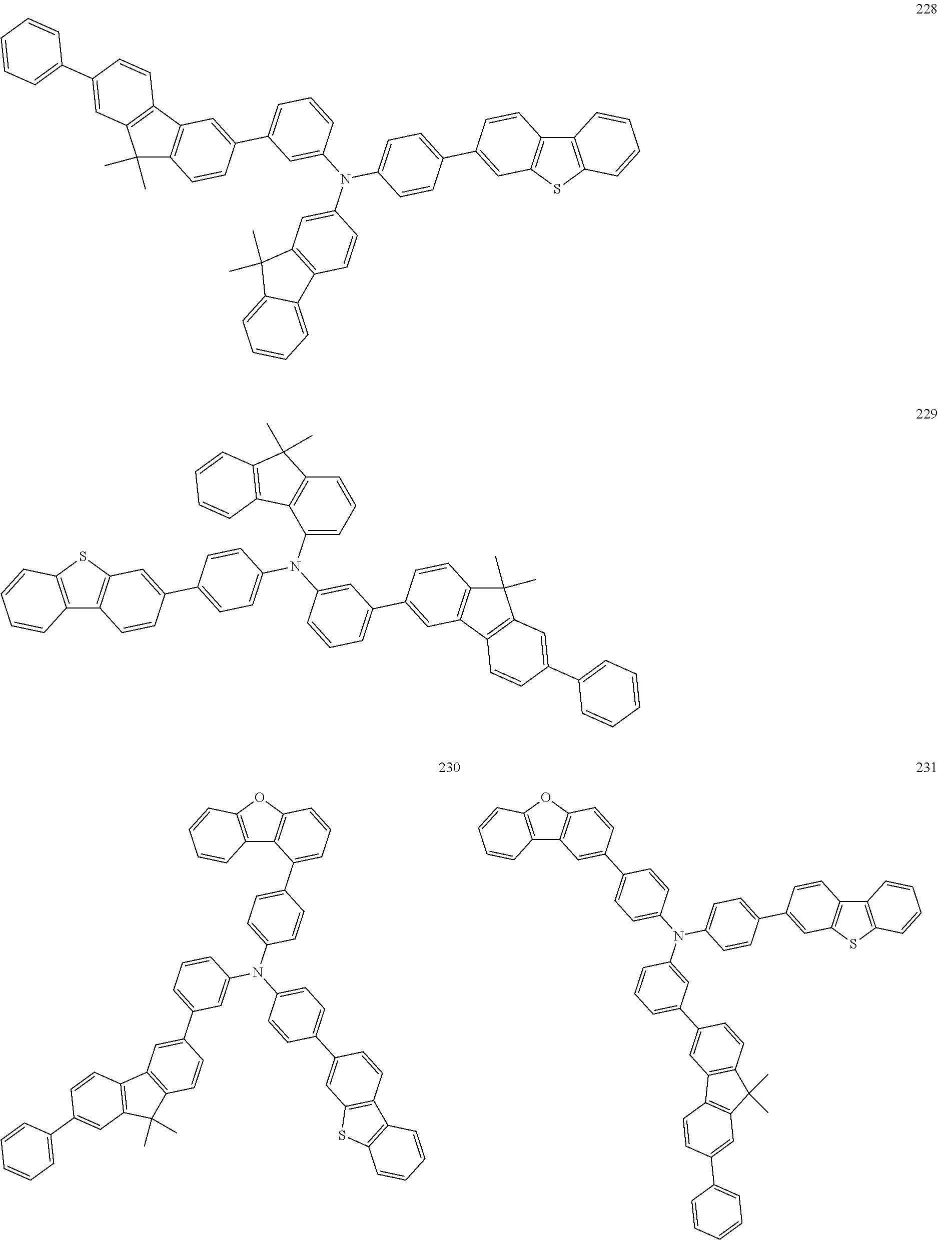 [SMILES: CC1(C)c2ccc(-c3cccc(N(c4ccc(-c5ccc6c(c5)sc5ccccc56)cc4)c4ccc5c(c4)C(C)(C)c4ccccc4-5)c3)cc2-c2ccc(-c3ccccc3)cc21 ; CC1(C)c2ccc(-c3cccc(N(c4ccc(-c5ccc6c(c5)sc5ccccc56)cc4)c4cccc5c4-c4ccccc4C5(C)C)c3)cc2-c2ccc(-c3ccccc3)cc21 ; CC1(C)c2ccc(-c3cccc(N(c4ccc(-c5ccc6c(c5)sc5ccccc56)cc4)c4ccc(-c5cccc6oc7ccccc7c56)cc4)c3)cc2-c2ccc(-c3ccccc3)cc21 ; CC1(C)c2ccc(-c3cccc(N(c4ccc(-c5ccc6c(c5)sc5ccccc56)cc4)c4ccc(-c5ccc6oc7ccccc7c6c5)cc4)c3)cc2-c2ccc(-c3ccccc3)cc21]

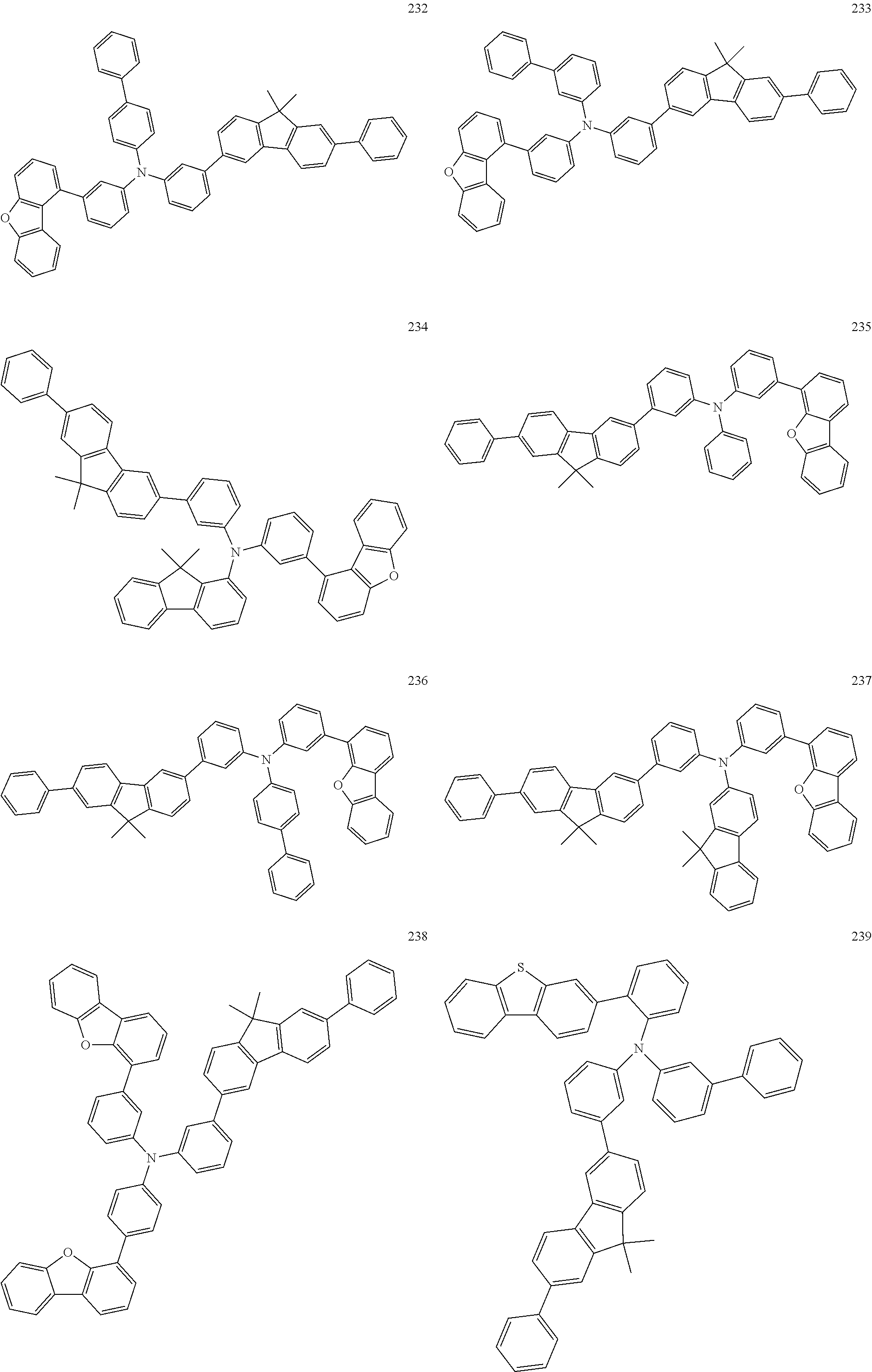

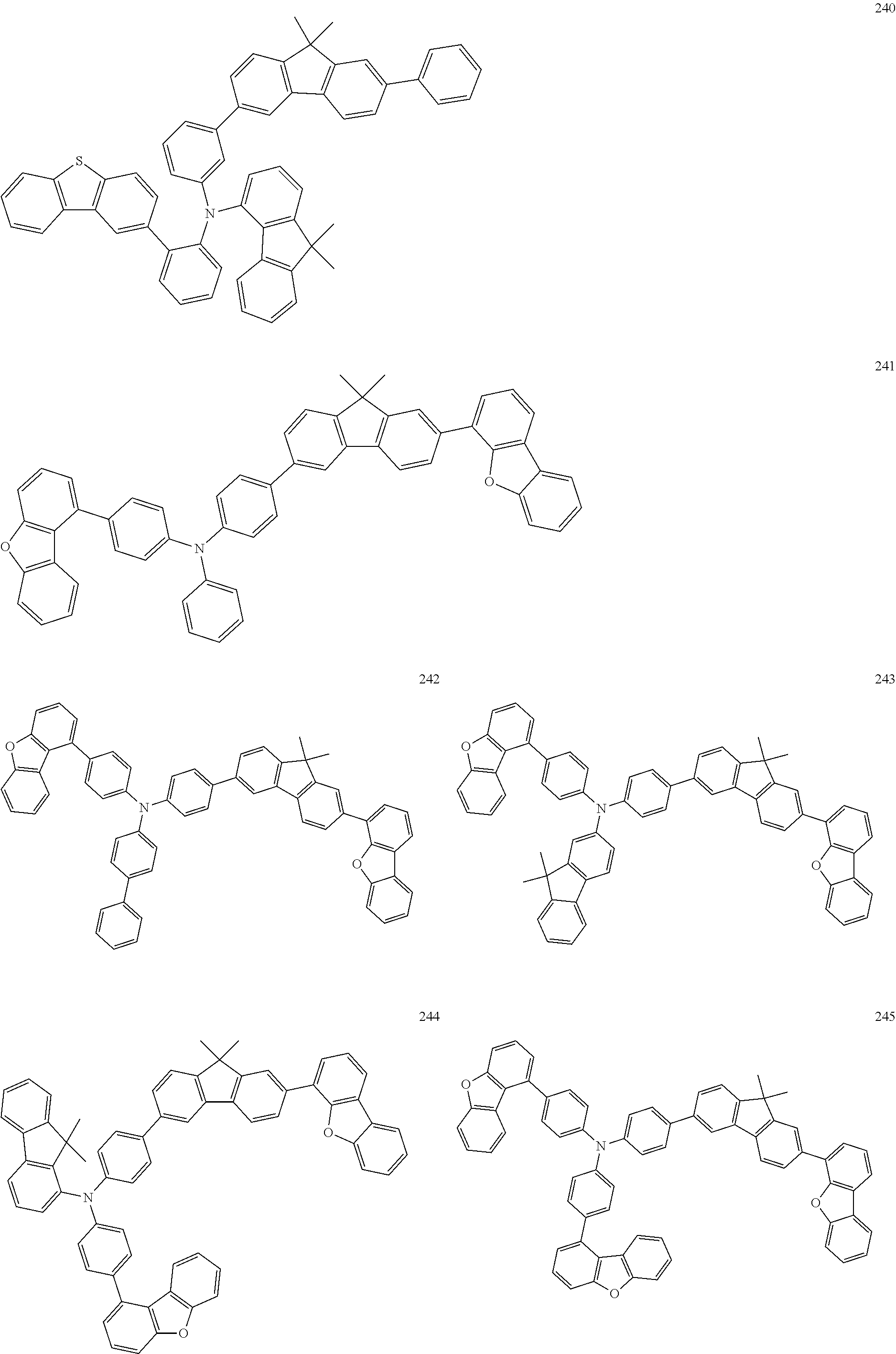

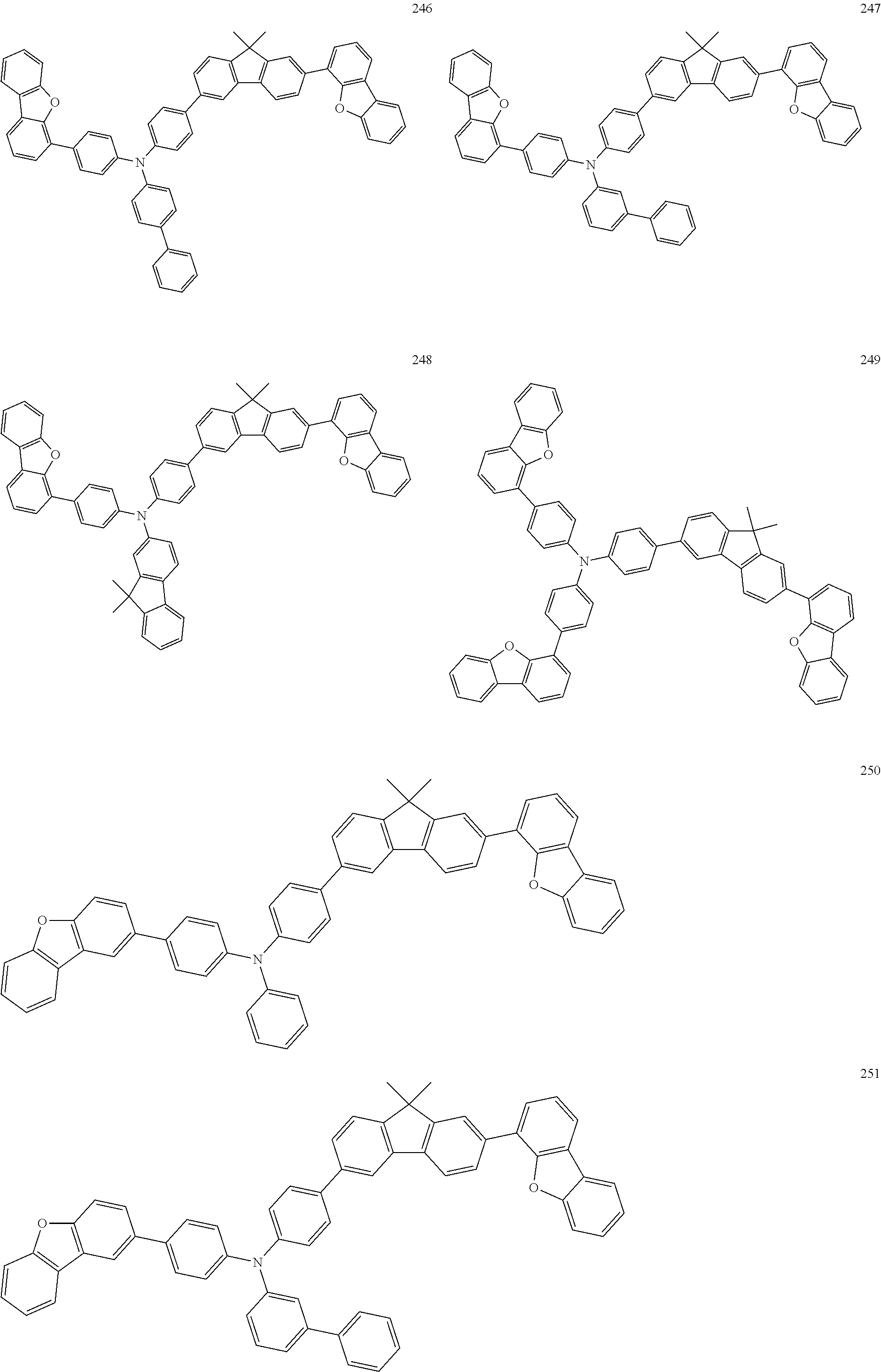

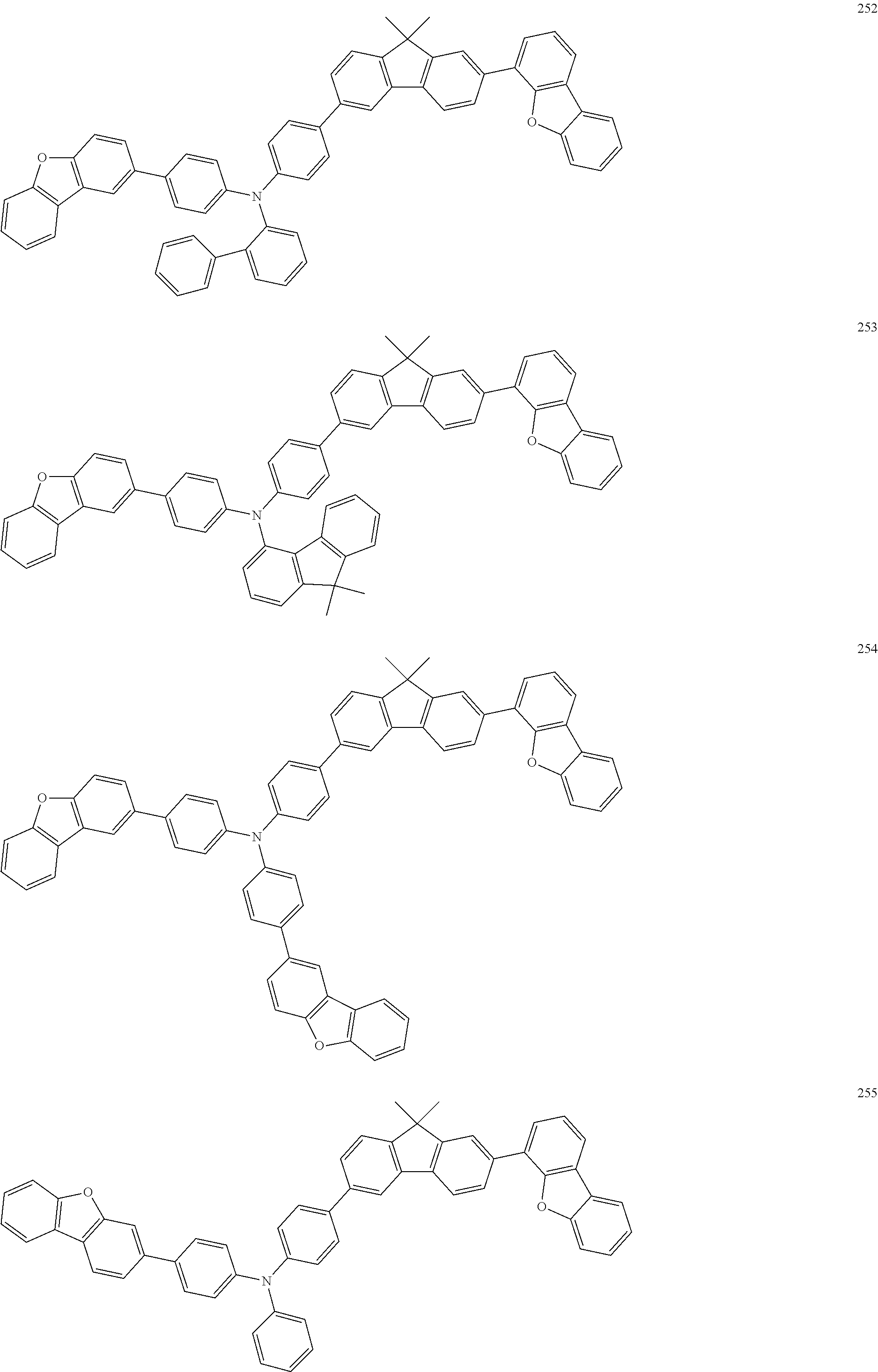 [SMILES: CC1(C)c2ccc(-c3ccc(N(c4ccc(-c5ccc6oc7ccccc7c6c5)cc4)c4ccccc4-c4ccccc4)cc3)cc2-c2ccc(-c3cccc4c3oc3ccccc34)cc21 ; CC1(C)c2ccc(-c3ccc(N(c4ccc(-c5ccc6oc7ccccc7c6c5)cc4)c4cccc5c4-c4ccccc4C5(C)C)cc3)cc2-c2ccc(-c3cccc4c3oc3ccccc34)cc21 ; CC1(C)c2ccc(-c3ccc(N(c4ccc(-c5ccc6oc7ccccc7c6c5)cc4)c4ccc(-c5ccc6oc7ccccc7c6c5)cc4)cc3)cc2-c2ccc(-c3cccc4c3oc3ccccc34)cc21 ; CC1(C)c2ccc(-c3ccc(N(c4ccccc4)c4ccc(-c5ccc6c(c5)oc5ccccc56)cc4)cc3)cc2-c2ccc(-c3cccc4c3oc3ccccc34)cc21]

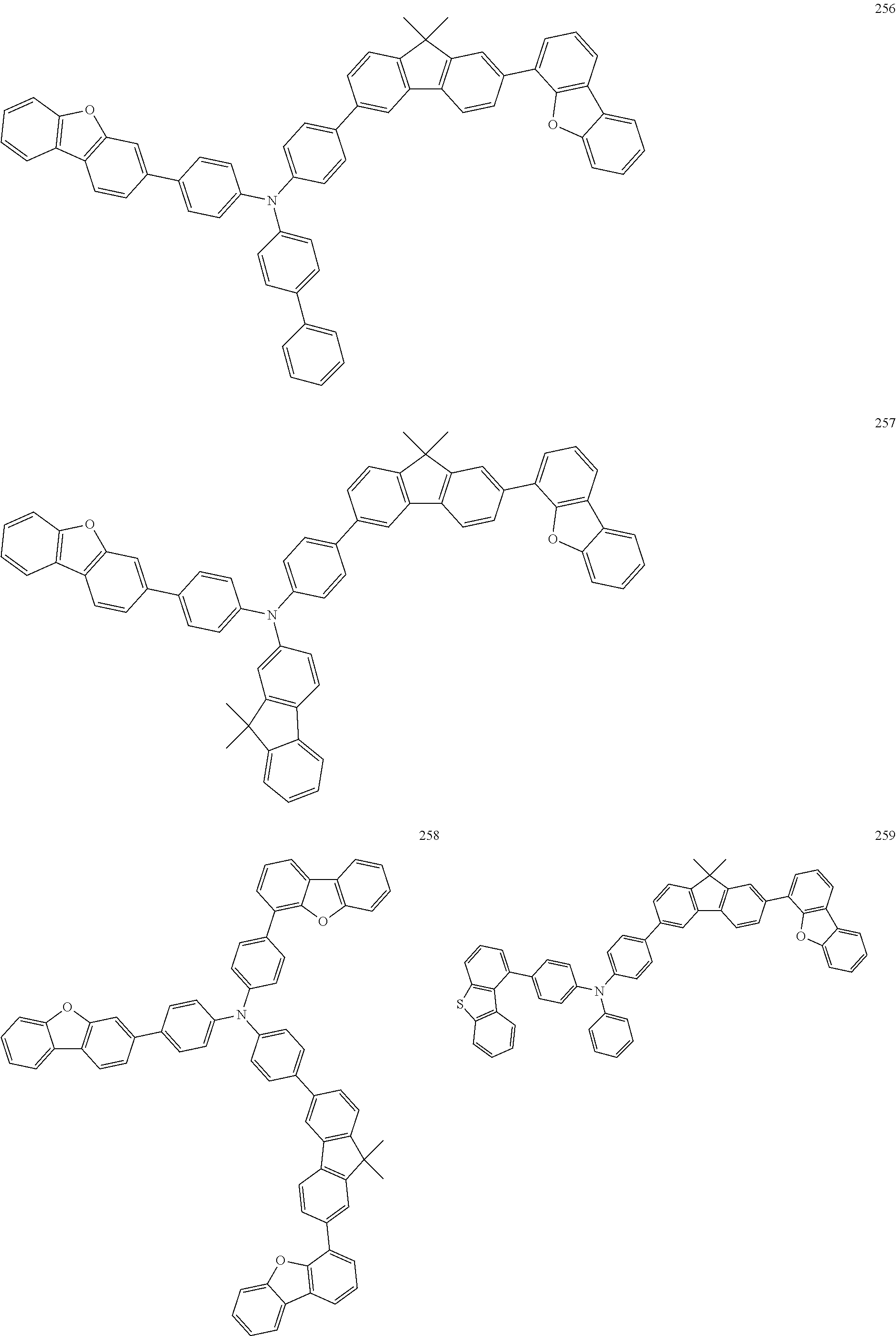 [SMILES: CC1(C)c2ccc(-c3ccc(N(c4ccc(-c5ccccc5)cc4)c4ccc(-c5ccc6c(c5)oc5ccccc56)cc4)cc3)cc2-c2ccc(-c3cccc4c3oc3ccccc34)cc21 ; CC1(C)c2ccc(-c3ccc(N(c4ccc(-c5ccc6c(c5)oc5ccccc56)cc4)c4ccc5c(c4)C(C)(C)c4ccccc4-5)cc3)cc2-c2ccc(-c3cccc4c3oc3ccccc34)cc21 ; CC1(C)c2ccc(-c3ccc(N(c4ccc(-c5ccc6c(c5)oc5ccccc56)cc4)c4ccc(-c5cccc6c5oc5ccccc56)cc4)cc3)cc2-c2ccc(-c3cccc4c3oc3ccccc34)cc21 ; CC1(C)c2ccc(-c3ccc(N(c4ccccc4)c4ccc(-c5cccc6sc7ccccc7c56)cc4)cc3)cc2-c2ccc(-c3cccc4c3oc3ccccc34)cc21]

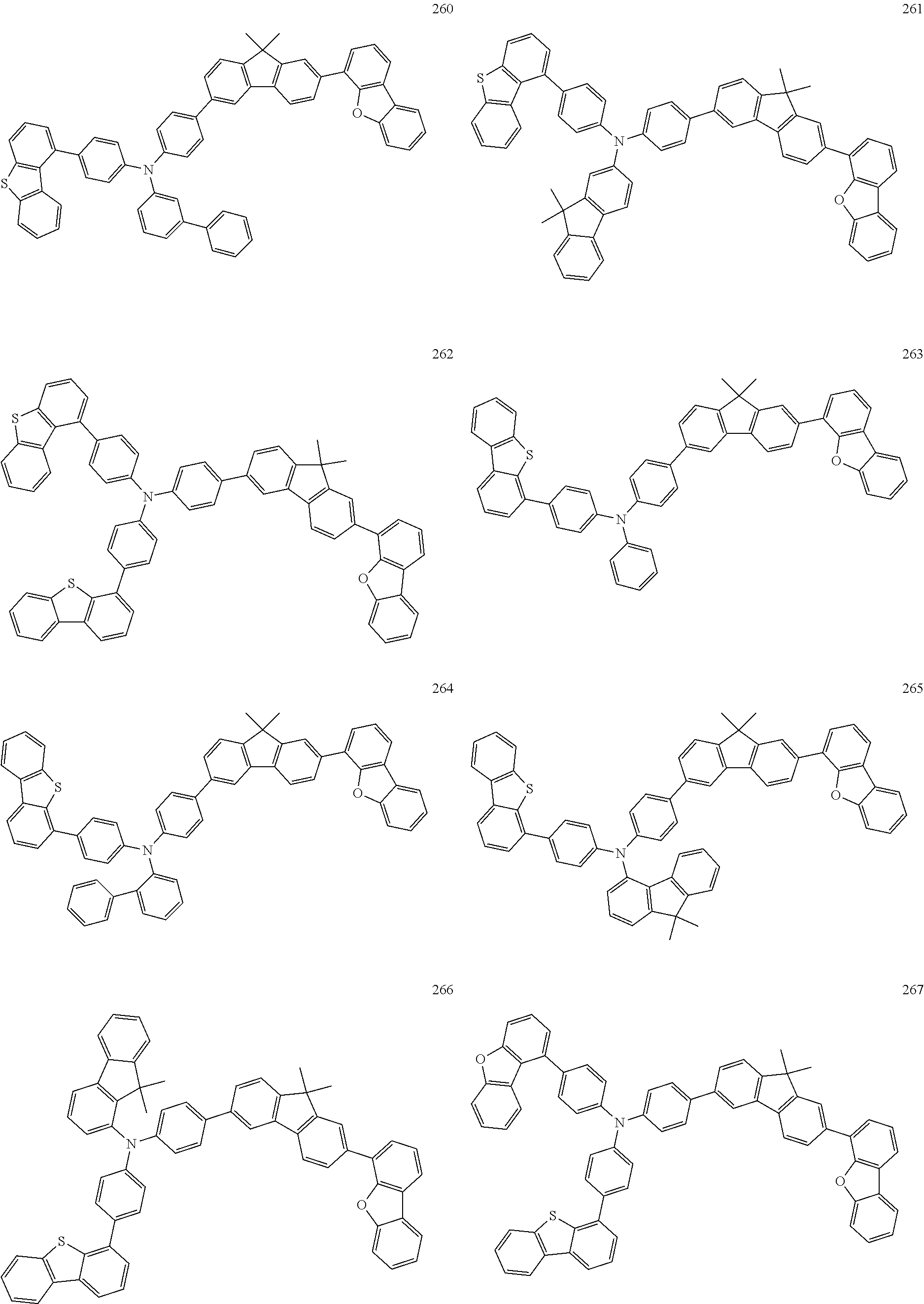

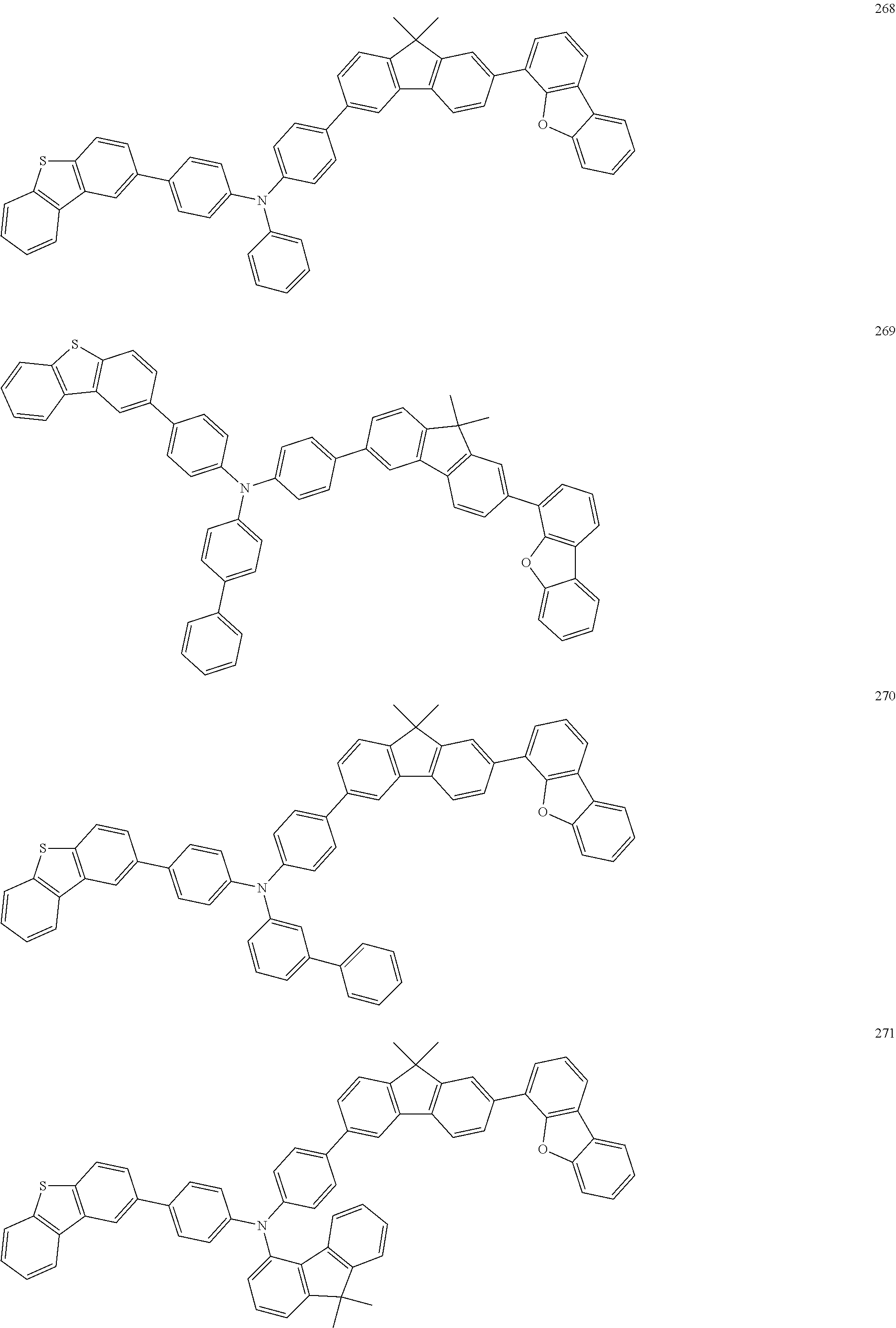

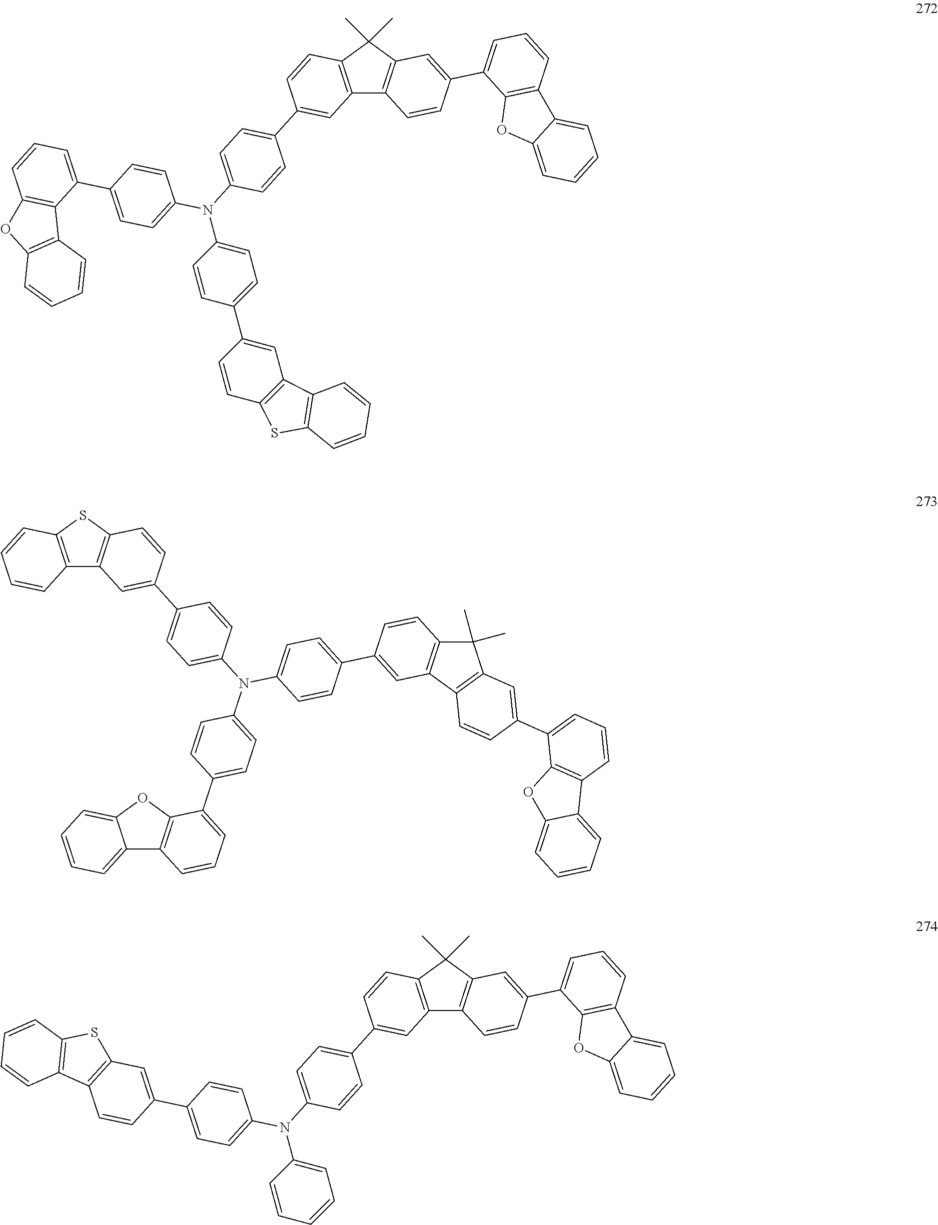 [SMILES: CC1(C)c2ccc(-c3ccc(N(c4ccc(-c5ccc6sc7ccccc7c6c5)cc4)c4ccc(-c5cccc6oc7ccccc7c56)cc4)cc3)cc2-c2ccc(-c3cccc4c3oc3ccccc34)cc21 ; CC1(C)c2ccc(-c3ccc(N(c4ccc(-c5ccc6sc7ccccc7c6c5)cc4)c4ccc(-c5cccc6c5oc5ccccc56)cc4)cc3)cc2-c2ccc(-c3cccc4c3oc3ccccc34)cc21 ; CC1(C)c2ccc(-c3ccc(N(c4ccccc4)c4ccc(-c5ccc6c(c5)sc5ccccc56)cc4)cc3)cc2-c2ccc(-c3cccc4c3oc3ccccc34)cc21]

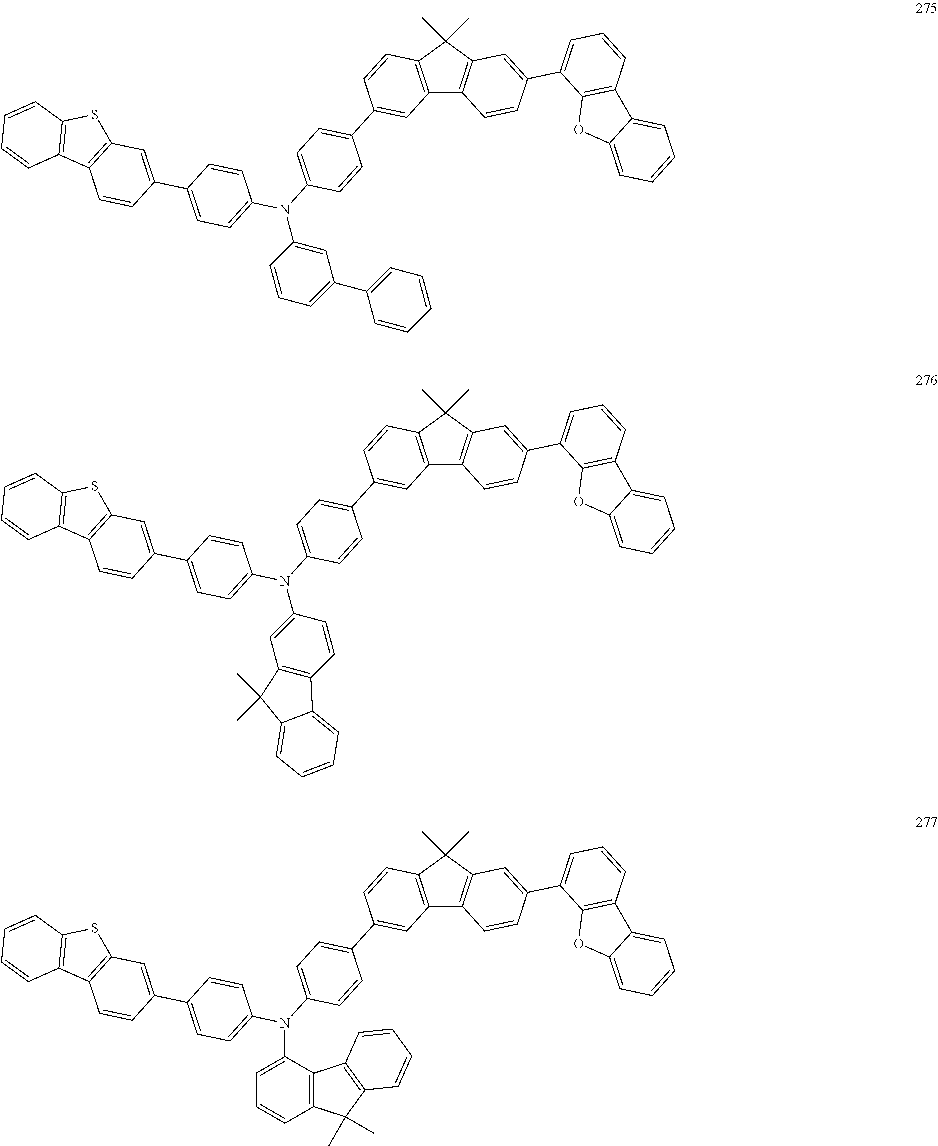 [SMILES: CC1(C)c2ccc(-c3ccc(N(c4ccc(-c5ccc6c(c5)sc5ccccc56)cc4)c4cccc(-c5ccccc5)c4)cc3)cc2-c2ccc(-c3cccc4c3oc3ccccc34)cc21 ; CC1(C)c2ccc(-c3ccc(N(c4ccc(-c5ccc6c(c5)sc5ccccc56)cc4)c4ccc5c(c4)C(C)(C)c4ccccc4-5)cc3)cc2-c2ccc(-c3cccc4c3oc3ccccc34)cc21 ; CC1(C)c2ccc(-c3ccc(N(c4ccc(-c5ccc6c(c5)sc5ccccc56)cc4)c4cccc5c4-c4ccccc4C5(C)C)cc3)cc2-c2ccc(-c3cccc4c3oc3ccccc34)cc21]

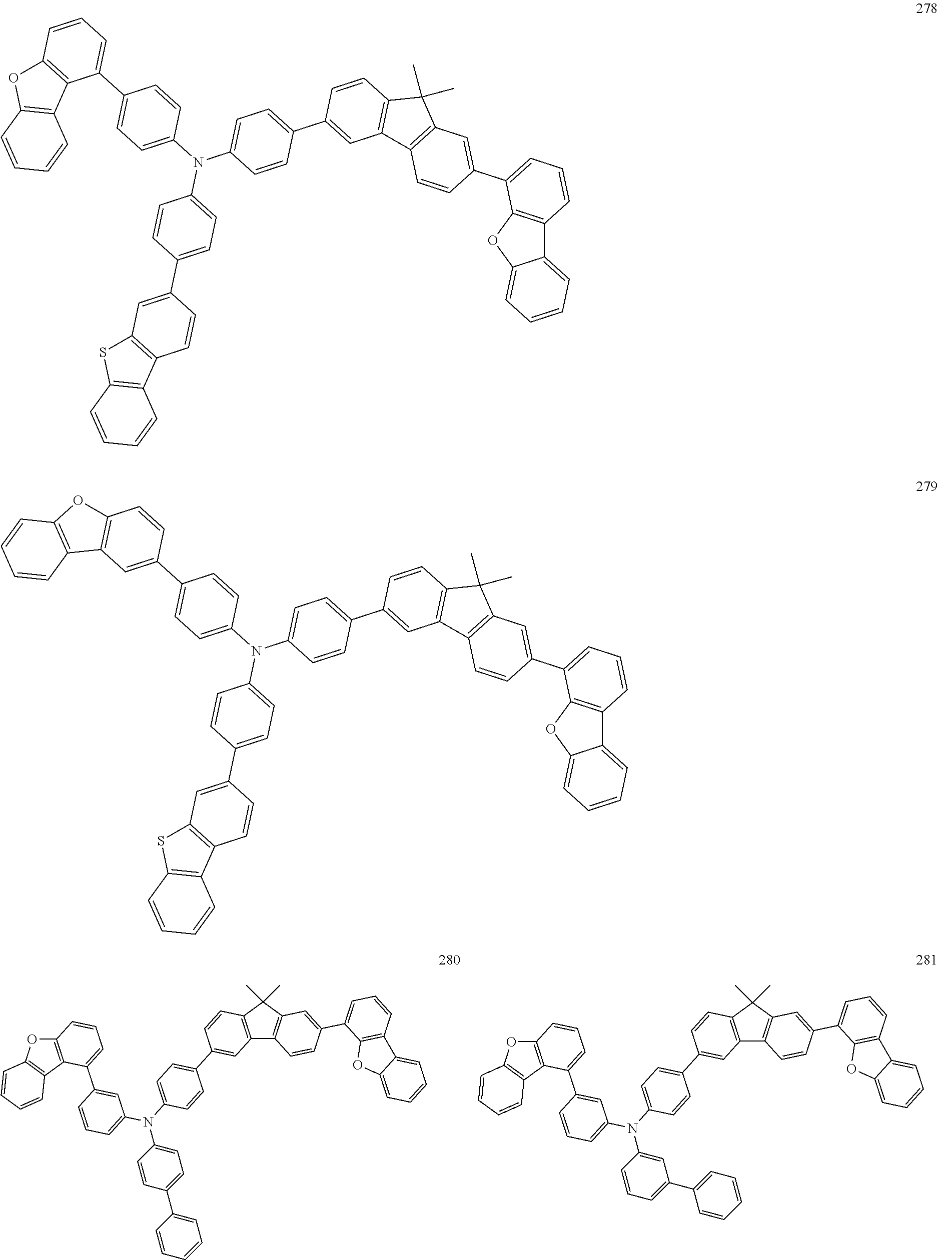 [SMILES: CC1(C)c2ccc(-c3ccc(N(c4ccc(-c5ccc6c(c5)sc5ccccc56)cc4)c4ccc(-c5cccc6oc7ccccc7c56)cc4)cc3)cc2-c2ccc(-c3cccc4c3oc3ccccc34)cc21 ; CC1(C)c2ccc(-c3ccc(N(c4ccc(-c5ccc6c(c5)sc5ccccc56)cc4)c4ccc(-c5ccc6oc7ccccc7c6c5)cc4)cc3)cc2-c2ccc(-c3cccc4c3oc3ccccc34)cc21 ; CC1(C)c2ccc(-c3ccc(N(c4ccc(-c5ccccc5)cc4)c4cccc(-c5cccc6oc7ccccc7c56)c4)cc3)cc2-c2ccc(-c3cccc4c3oc3ccccc34)cc21 ; CC1(C)c2ccc(-c3ccc(N(c4cccc(-c5ccccc5)c4)c4cccc(-c5cccc6oc7ccccc7c56)c4)cc3)cc2-c2ccc(-c3cccc4c3oc3ccccc34)cc21]

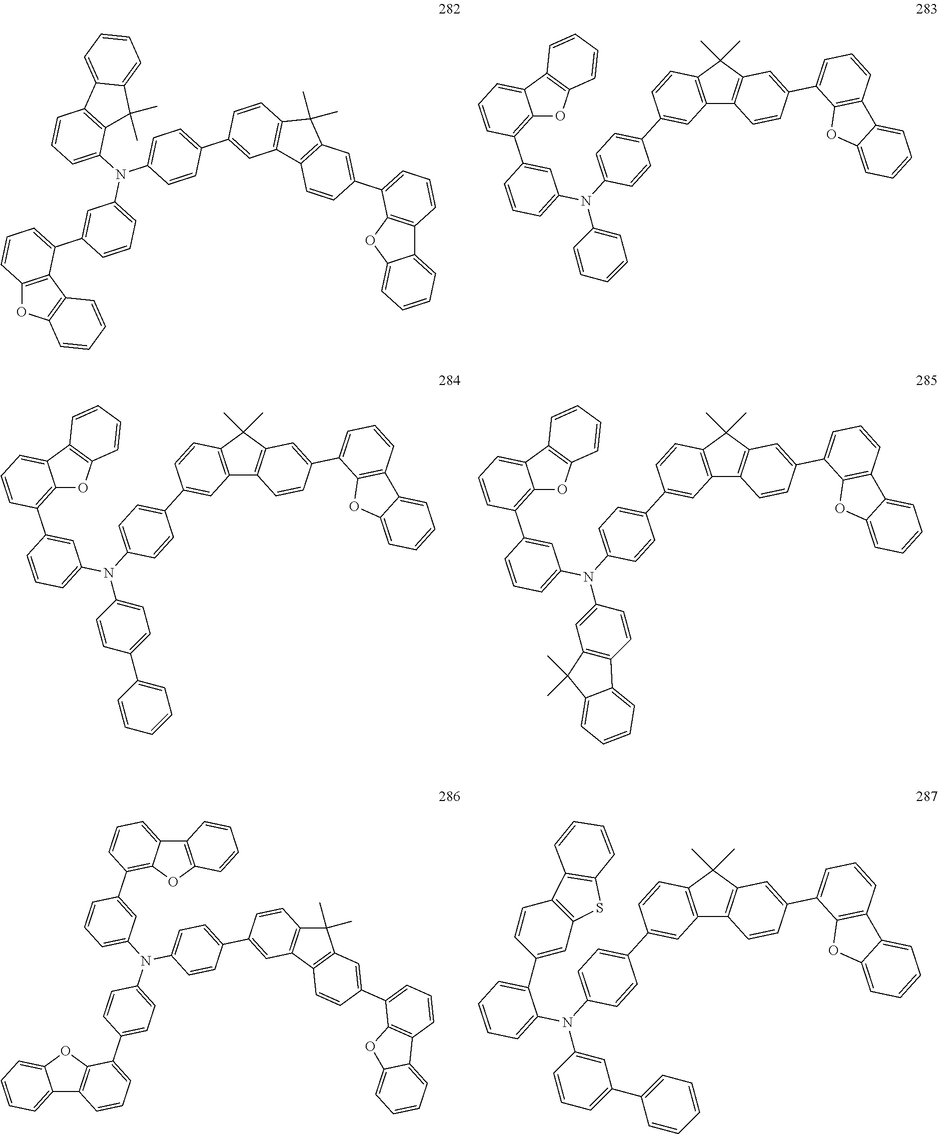

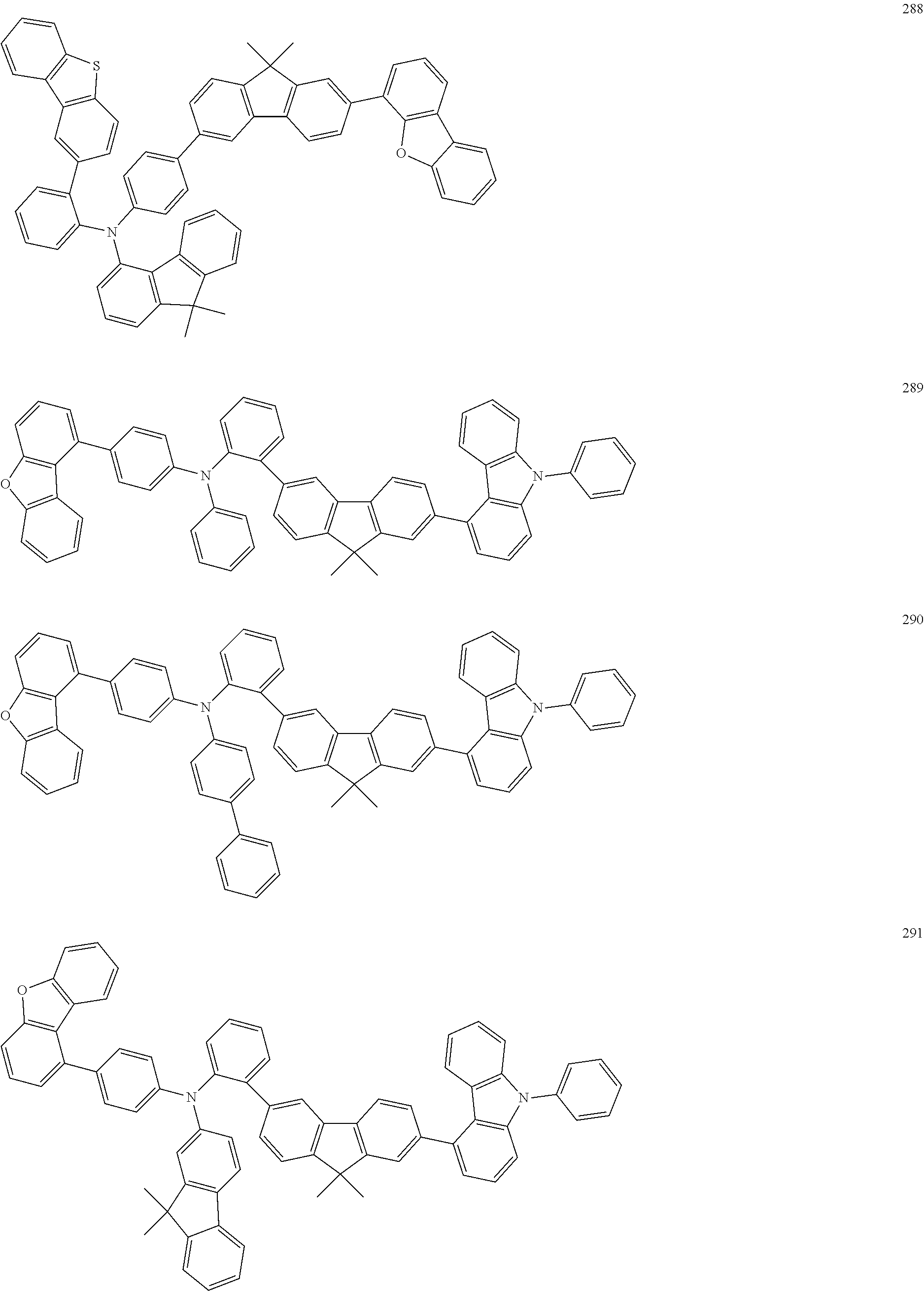 [SMILES: CC1(C)c2ccc(-c3ccc(N(c4ccccc4-c4ccc5sc6ccccc6c5c4)c4cccc5c4-c4ccccc4C5(C)C)cc3)cc2-c2ccc(-c3cccc4c3oc3ccccc34)cc21 ; CC1(C)c2ccc(-c3ccccc3N(c3ccccc3)c3ccc(-c4cccc5oc6ccccc6c45)cc3)cc2-c2ccc(-c3cccc4c3c3ccccc3n4-c3ccccc3)cc21 ; CC1(C)c2ccc(-c3ccccc3N(c3ccc(-c4ccccc4)cc3)c3ccc(-c4cccc5oc6ccccc6c45)cc3)cc2-c2ccc(-c3cccc4c3c3ccccc3n4-c3ccccc3)cc21 ; CC1(C)c2ccc(-c3ccccc3N(c3ccc(-c4cccc5oc6ccccc6c45)cc3)c3ccc4c(c3)C(C)(C)c3ccccc3-4)cc2-c2ccc(-c3cccc4c3c3ccccc3n4-c3ccccc3)cc21]

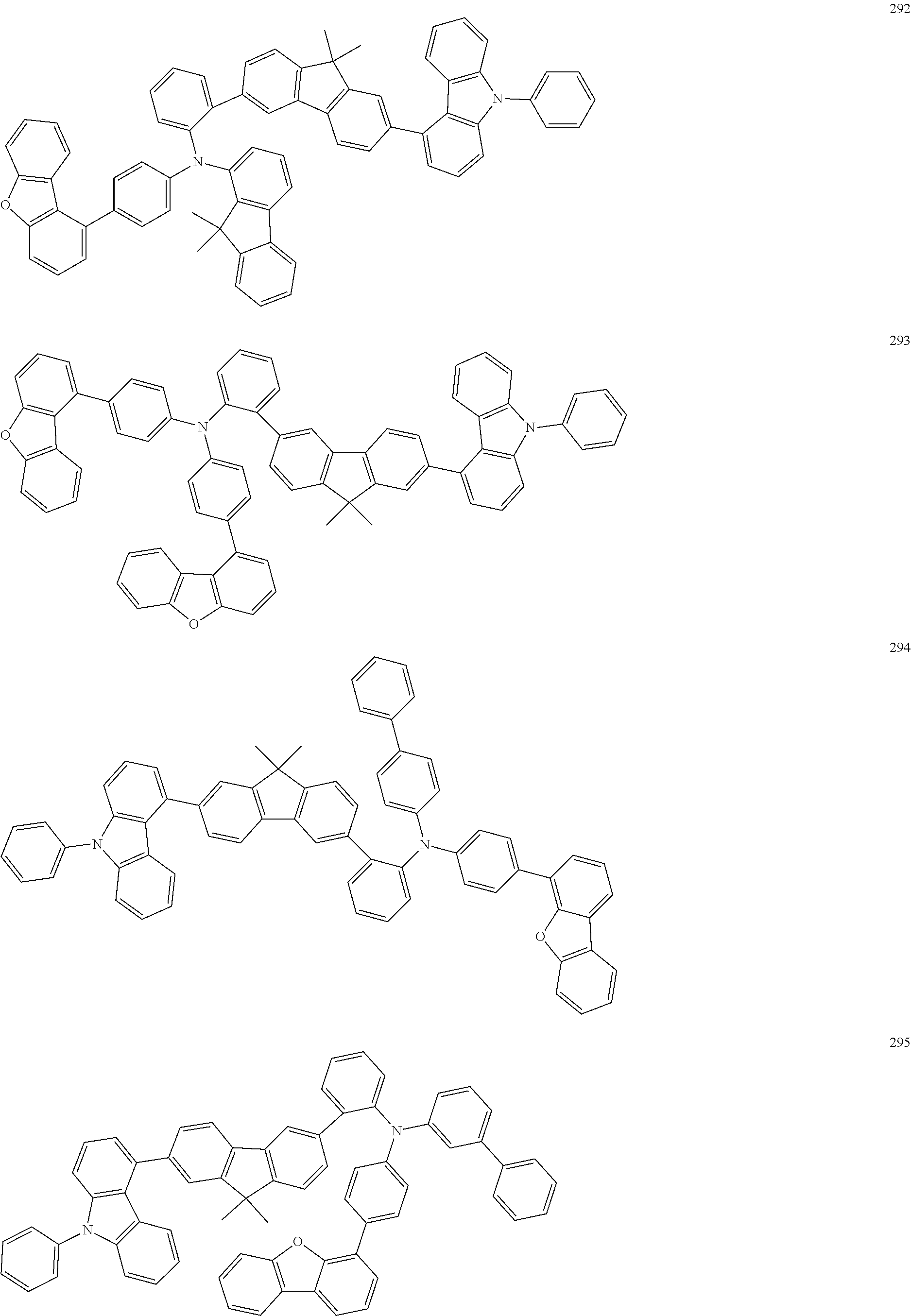 [SMILES: CC1(C)c2ccc(-c3ccccc3N(c3ccc(-c4cccc5oc6ccccc6c45)cc3)c3cccc4c3C(C)(C)c3ccccc3-4)cc2-c2ccc(-c3cccc4c3c3ccccc3n4-c3ccccc3)cc21 ; CC1(C)c2ccc(-c3ccccc3N(c3ccc(-c4cccc5oc6ccccc6c45)cc3)c3ccc(-c4cccc5oc6ccccc6c45)cc3)cc2-c2ccc(-c3cccc4c3c3ccccc3n4-c3ccccc3)cc21 ; CC1(C)c2ccc(-c3ccccc3N(c3ccc(-c4ccccc4)cc3)c3ccc(-c4cccc5c4oc4ccccc45)cc3)cc2-c2ccc(-c3cccc4c3c3ccccc3n4-c3ccccc3)cc21 ; CC1(C)c2ccc(-c3ccccc3N(c3ccc(-c4cccc5c4oc4ccccc45)cc3)c3cccc(-c4ccccc4)c3)cc2-c2ccc(-c3cccc4c3c3ccccc3n4-c3ccccc3)cc21]

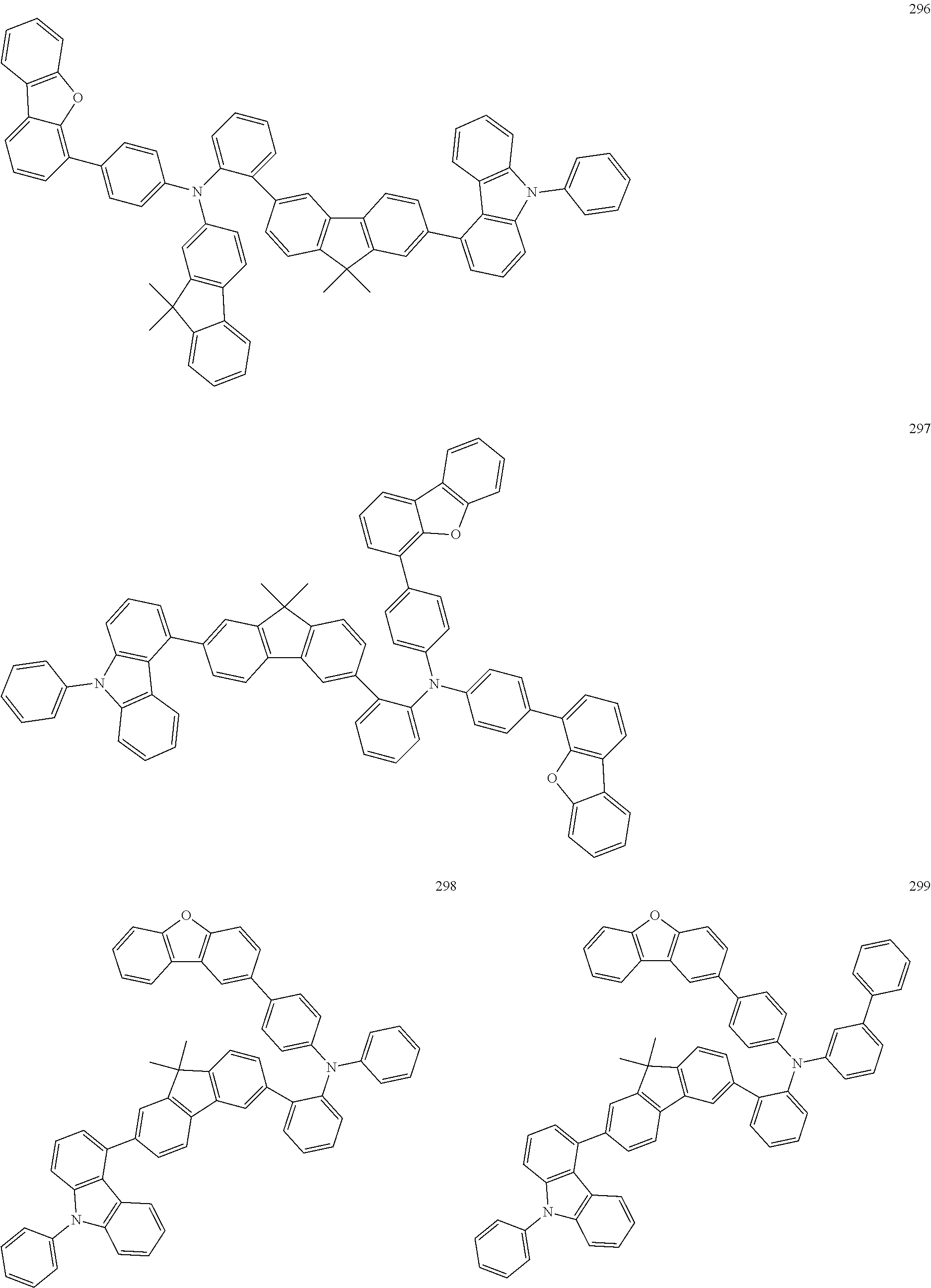 [SMILES: CC1(C)c2ccc(-c3ccccc3N(c3ccc(-c4cccc5c4oc4ccccc45)cc3)c3ccc4c(c3)C(C)(C)c3ccccc3-4)cc2-c2ccc(-c3cccc4c3c3ccccc3n4-c3ccccc3)cc21 ; CC1(C)c2ccc(-c3ccccc3N(c3ccc(-c4cccc5c4oc4ccccc45)cc3)c3ccc(-c4cccc5c4oc4ccccc45)cc3)cc2-c2ccc(-c3cccc4c3c3ccccc3n4-c3ccccc3)cc21 ; CC1(C)c2ccc(-c3ccccc3N(c3ccccc3)c3ccc(-c4ccc5oc6ccccc6c5c4)cc3)cc2-c2ccc(-c3cccc4c3c3ccccc3n4-c3ccccc3)cc21 ; CC1(C)c2ccc(-c3ccccc3N(c3ccc(-c4ccc5oc6ccccc6c5c4)cc3)c3cccc(-c4ccccc4)c3)cc2-c2ccc(-c3cccc4c3c3ccccc3n4-c3ccccc3)cc21]

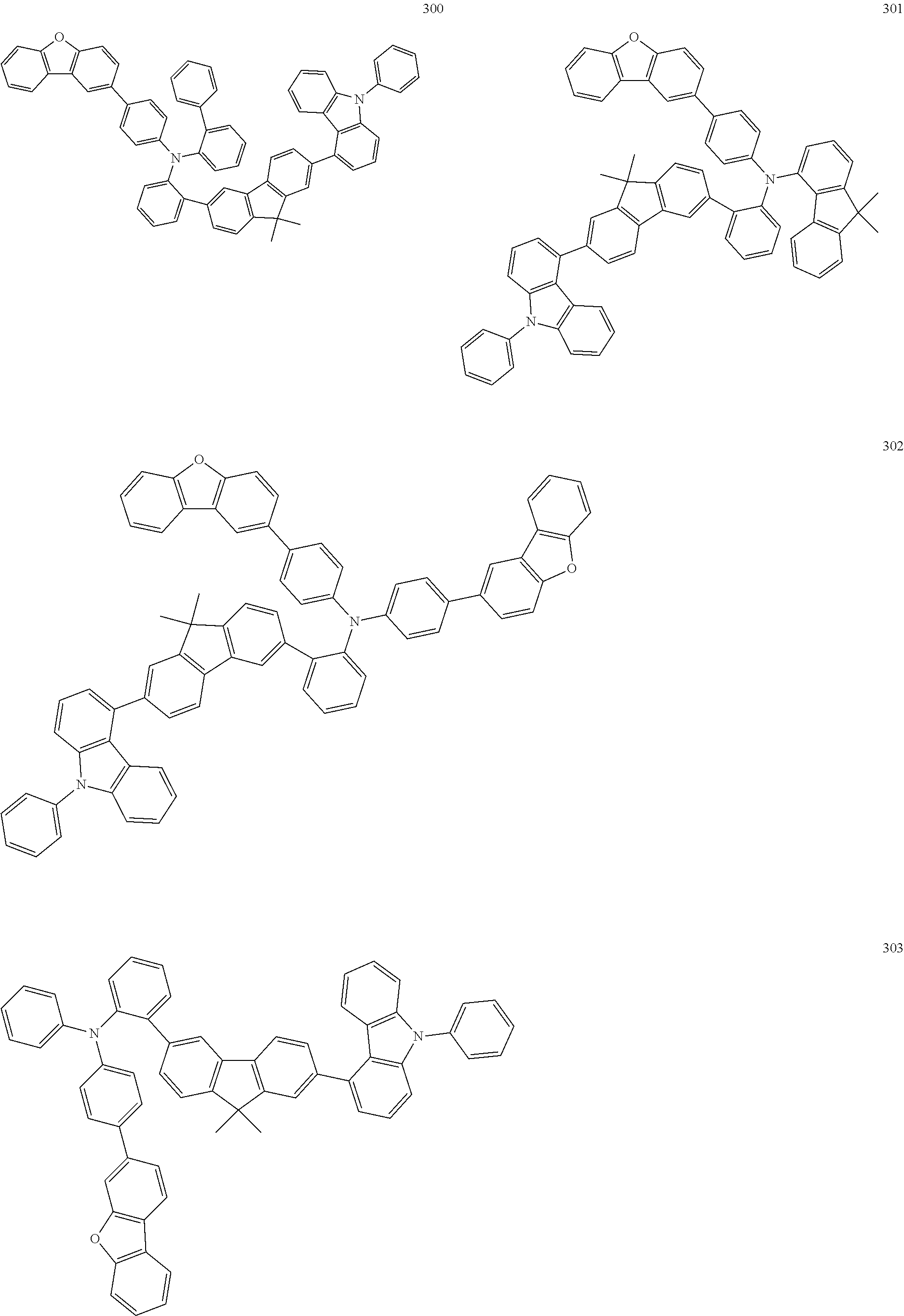 [SMILES: CC1(C)c2ccc(-c3ccccc3N(c3ccc(-c4ccc5oc6ccccc6c5c4)cc3)c3ccccc3-c3ccccc3)cc2-c2ccc(-c3cccc4c3c3ccccc3n4-c3ccccc3)cc21 ; CC1(C)c2ccc(-c3ccccc3N(c3ccc(-c4ccc5oc6ccccc6c5c4)cc3)c3cccc4c3-c3ccccc3C4(C)C)cc2-c2ccc(-c3cccc4c3c3ccccc3n4-c3ccccc3)cc21 ; CC1(C)c2ccc(-c3ccccc3N(c3ccc(-c4ccc5oc6ccccc6c5c4)cc3)c3ccc(-c4ccc5oc6ccccc6c5c4)cc3)cc2-c2ccc(-c3cccc4c3c3ccccc3n4-c3ccccc3)cc21 ; CC1(C)c2ccc(-c3ccccc3N(c3ccccc3)c3ccc(-c4ccc5c(c4)oc4ccccc45)cc3)cc2-c2ccc(-c3cccc4c3c3ccccc3n4-c3ccccc3)cc21]

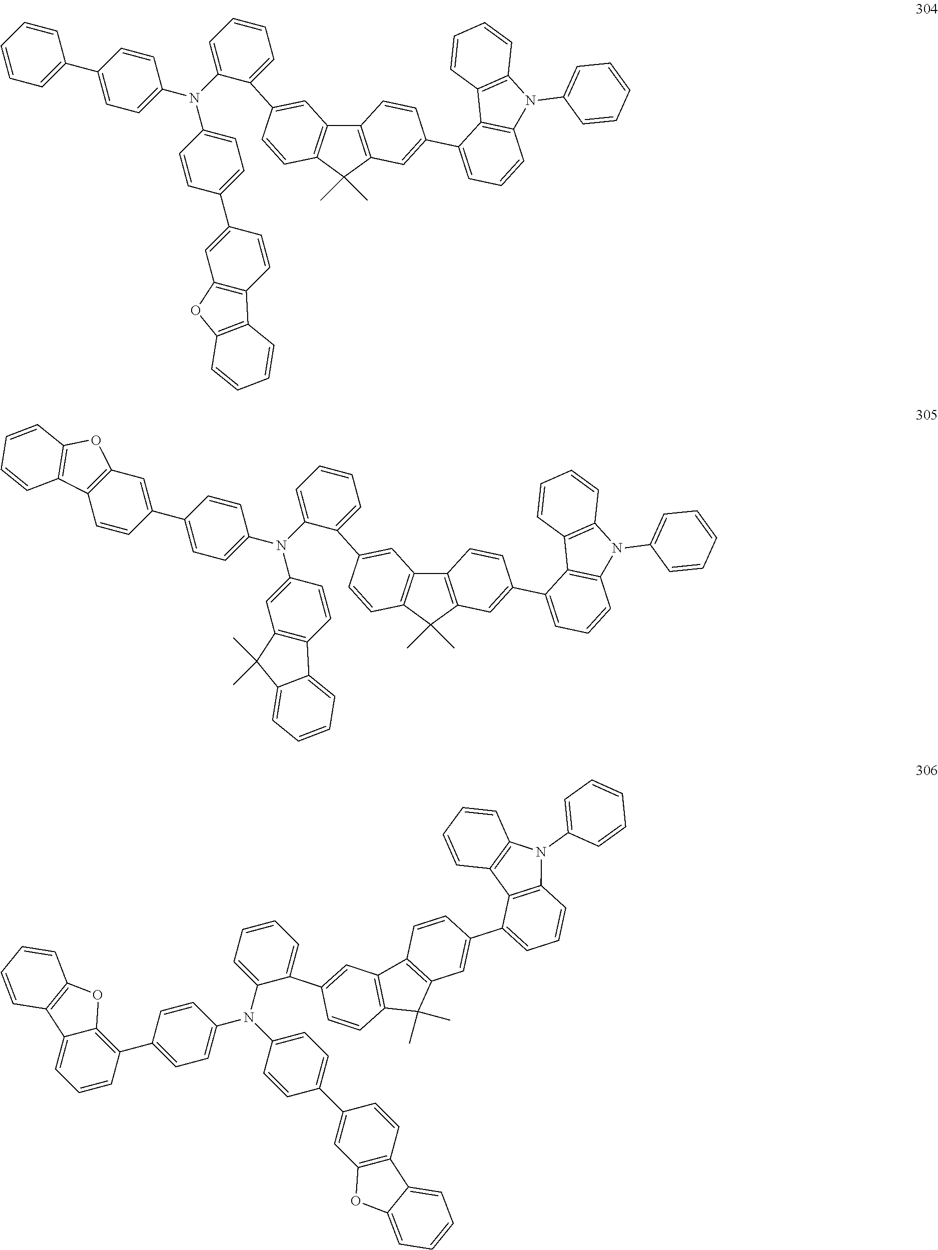 [SMILES: CC1(C)c2ccc(-c3ccccc3N(c3ccc(-c4ccccc4)cc3)c3ccc(-c4ccc5c(c4)oc4ccccc45)cc3)cc2-c2ccc(-c3cccc4c3c3ccccc3n4-c3ccccc3)cc21 ; CC1(C)c2ccc(-c3ccccc3N(c3ccc(-c4ccc5c(c4)oc4ccccc45)cc3)c3ccc4c(c3)C(C)(C)c3ccccc3-4)cc2-c2ccc(-c3cccc4c3c3ccccc3n4-c3ccccc3)cc21 ; CC1(C)c2ccc(-c3ccccc3N(c3ccc(-c4ccc5c(c4)oc4ccccc45)cc3)c3ccc(-c4cccc5c4oc4ccccc45)cc3)cc2-c2ccc(-c3cccc4c3c3ccccc3n4-c3ccccc3)cc21]

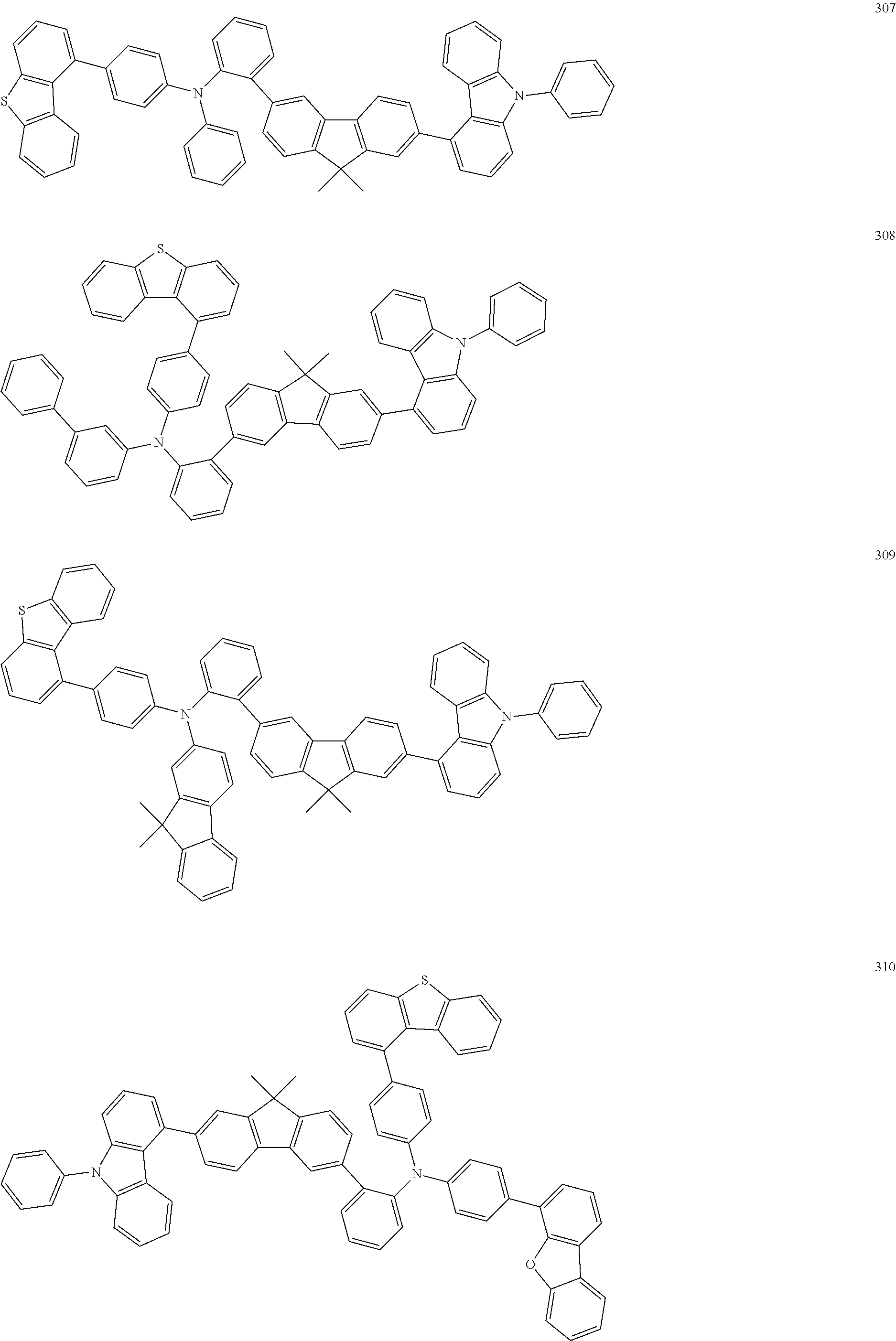 [SMILES: CC1(C)c2ccc(-c3ccccc3N(c3ccccc3)c3ccc(-c4cccc5sc6ccccc6c45)cc3)cc2-c2ccc(-c3cccc4c3c3ccccc3n4-c3ccccc3)cc21 ; CC1(C)c2ccc(-c3ccccc3N(c3ccc(-c4cccc5sc6ccccc6c45)cc3)c3cccc(-c4ccccc4)c3)cc2-c2ccc(-c3cccc4c3c3ccccc3n4-c3ccccc3)cc21 ; CC1(C)c2ccc(-c3ccccc3N(c3ccc(-c4cccc5sc6ccccc6c45)cc3)c3ccc4c(c3)C(C)(C)c3ccccc3-4)cc2-c2ccc(-c3cccc4c3c3ccccc3n4-c3ccccc3)cc21 ; CC1(C)c2ccc(-c3ccccc3N(c3ccc(-c4cccc5c4oc4ccccc45)cc3)c3ccc(-c4cccc5sc6ccccc6c45)cc3)cc2-c2ccc(-c3cccc4c3c3ccccc3n4-c3ccccc3)cc21]

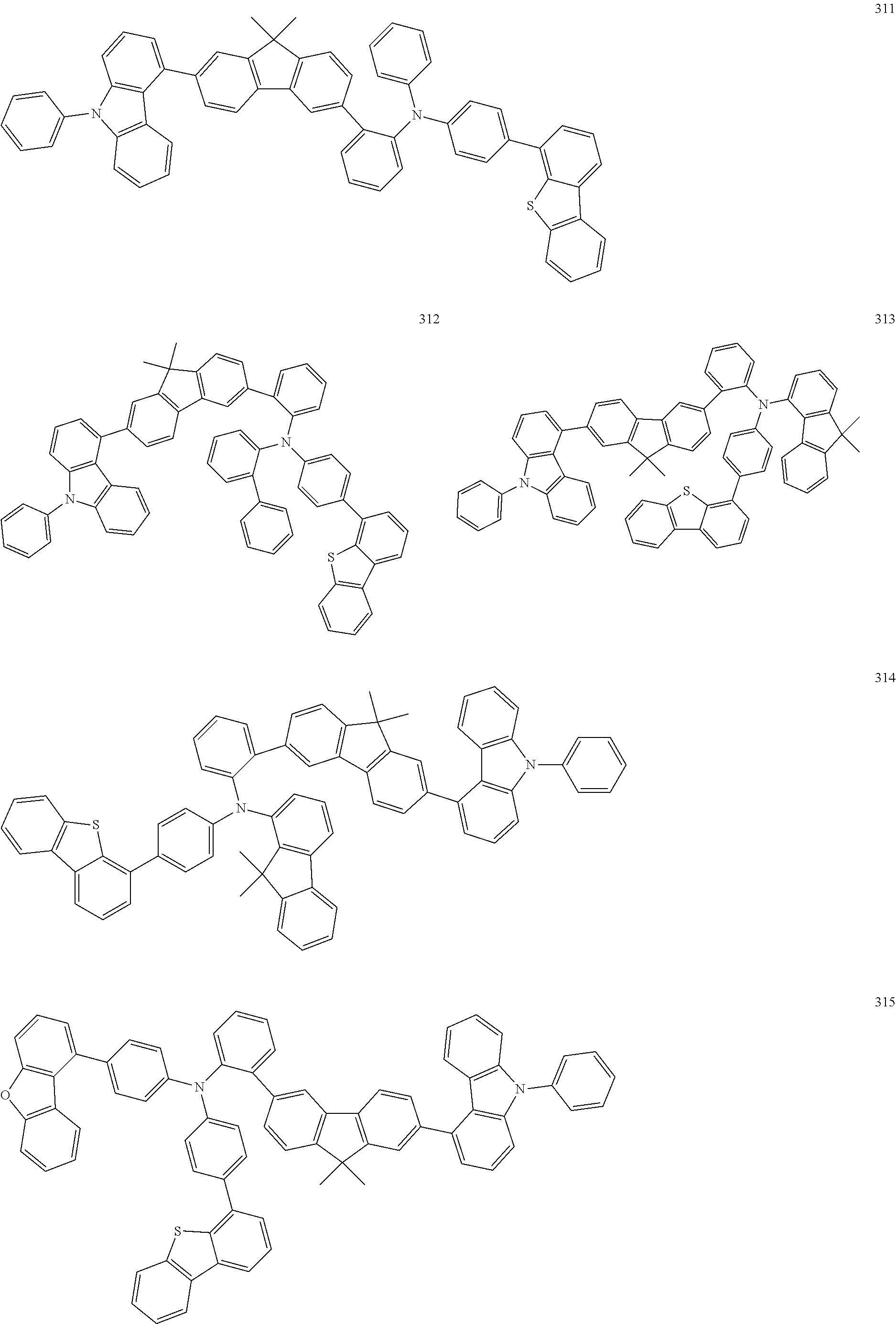

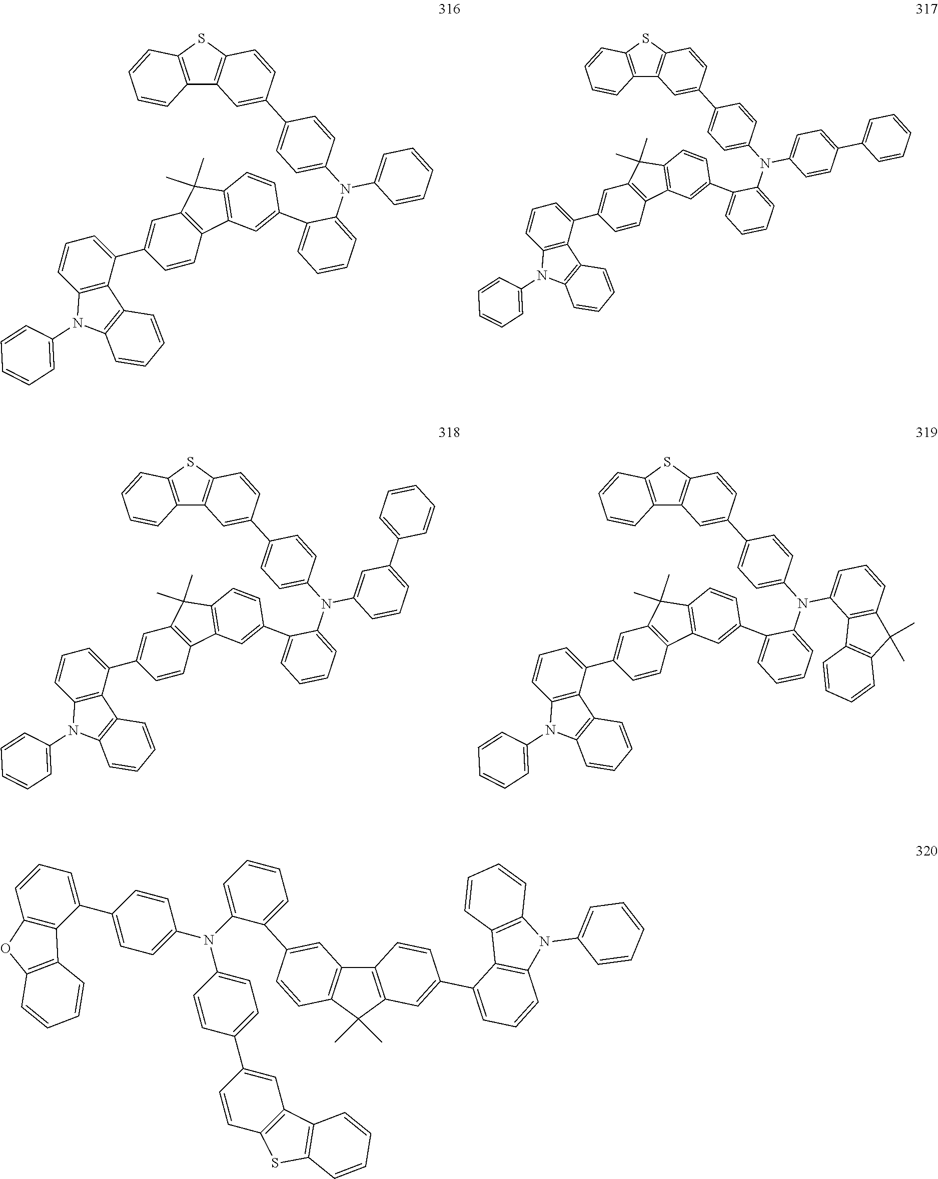

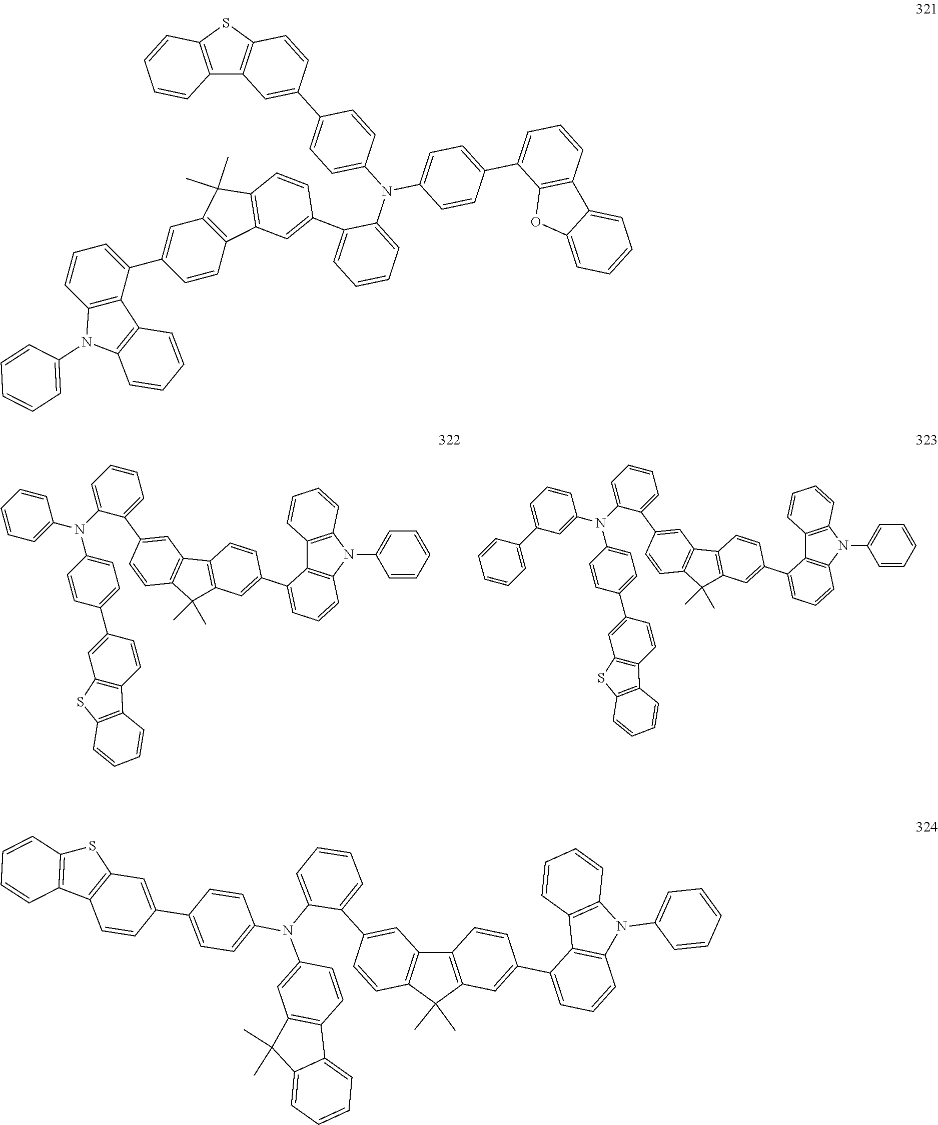 [SMILES: CC1(C)c2ccc(-c3ccccc3N(c3ccc(-c4ccc5sc6ccccc6c5c4)cc3)c3ccc(-c4cccc5c4oc4ccccc45)cc3)cc2-c2ccc(-c3cccc4c3c3ccccc3n4-c3ccccc3)cc21 ; CC1(C)c2ccc(-c3ccccc3N(c3ccccc3)c3ccc(-c4ccc5c(c4)sc4ccccc45)cc3)cc2-c2ccc(-c3cccc4c3c3ccccc3n4-c3ccccc3)cc21 ; CC1(C)c2ccc(-c3ccccc3N(c3ccc(-c4ccc5c(c4)sc4ccccc45)cc3)c3cccc(-c4ccccc4)c3)cc2-c2ccc(-c3cccc4c3c3ccccc3n4-c3ccccc3)cc21 ; CC1(C)c2ccc(-c3ccccc3N(c3ccc(-c4ccc5c(c4)sc4ccccc45)cc3)c3ccc4c(c3)C(C)(C)c3ccccc3-4)cc2-c2ccc(-c3cccc4c3c3ccccc3n4-c3ccccc3)cc21]

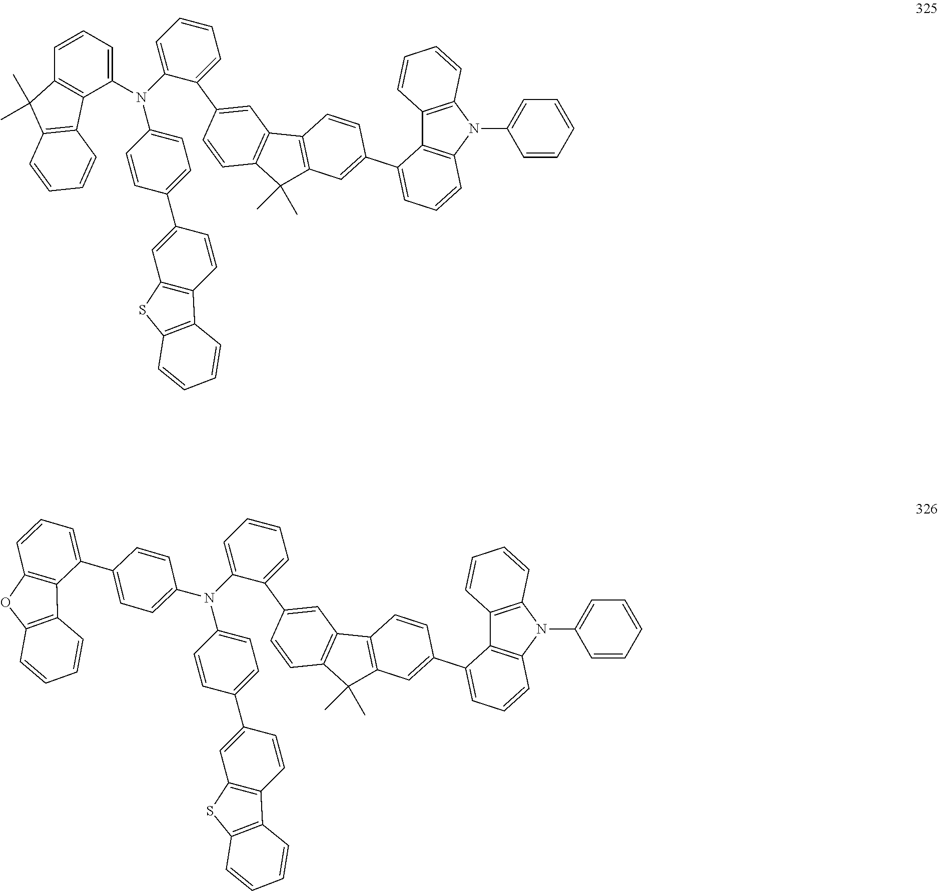 [SMILES: CC1(C)c2ccc(-c3ccccc3N(c3ccc(-c4ccc5c(c4)sc4ccccc45)cc3)c3cccc4c3-c3ccccc3C4(C)C)cc2-c2ccc(-c3cccc4c3c3ccccc3n4-c3ccccc3)cc21 ; CC1(C)c2ccc(-c3ccccc3N(c3ccc(-c4ccc5c(c4)sc4ccccc45)cc3)c3ccc(-c4cccc5oc6ccccc6c45)cc3)cc2-c2ccc(-c3cccc4c3c3ccccc3n4-c3ccccc3)cc21]

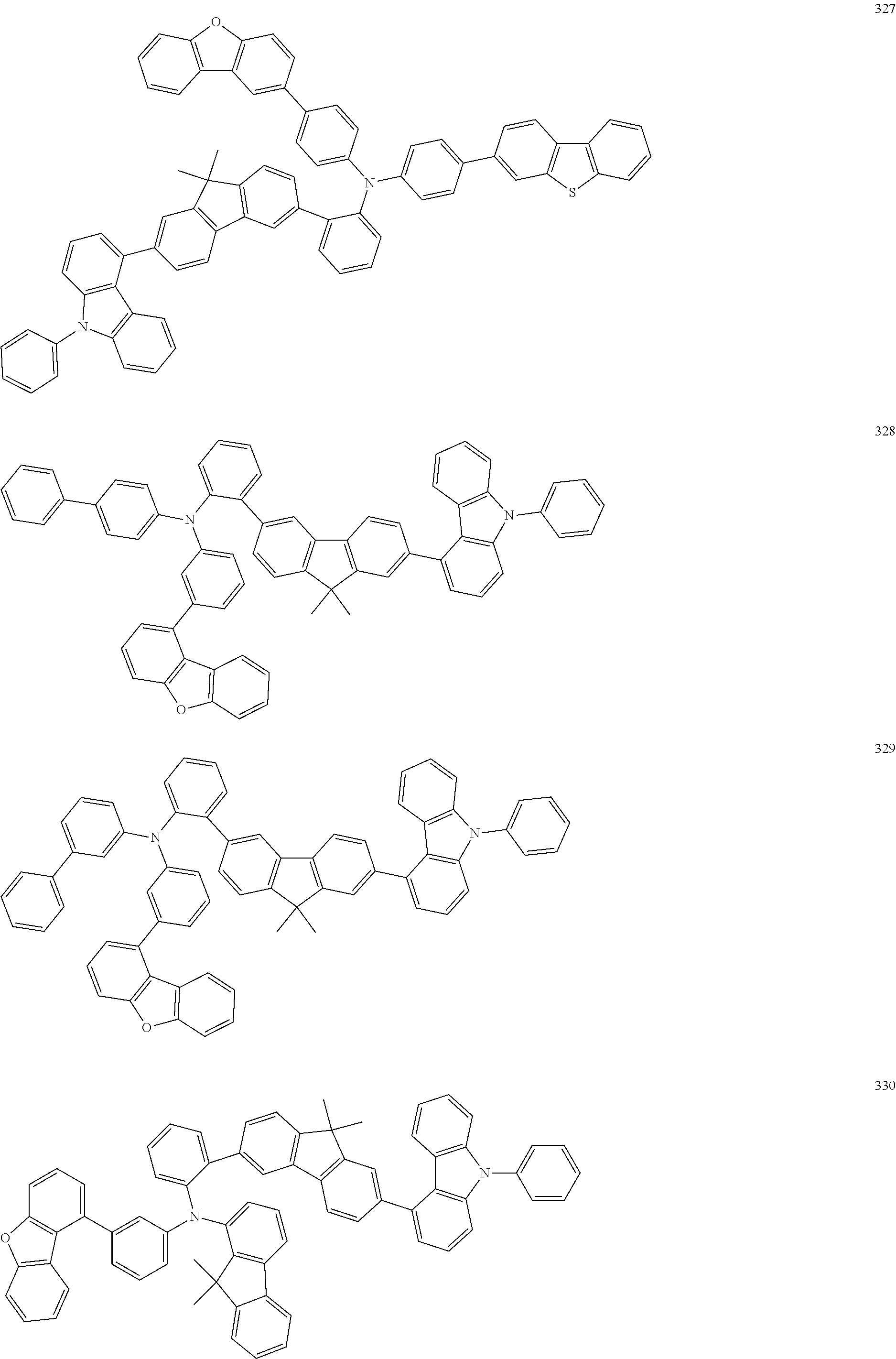

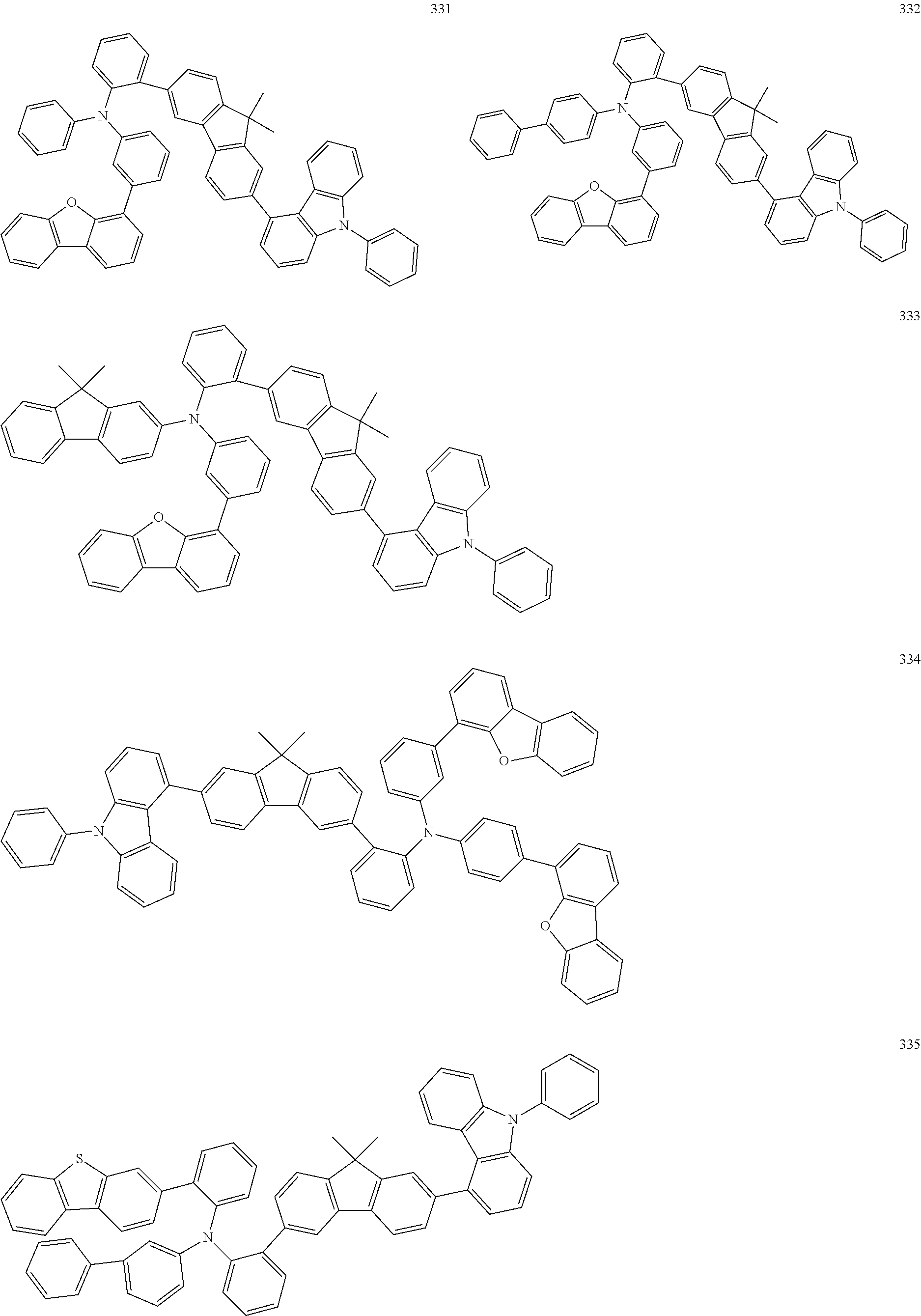

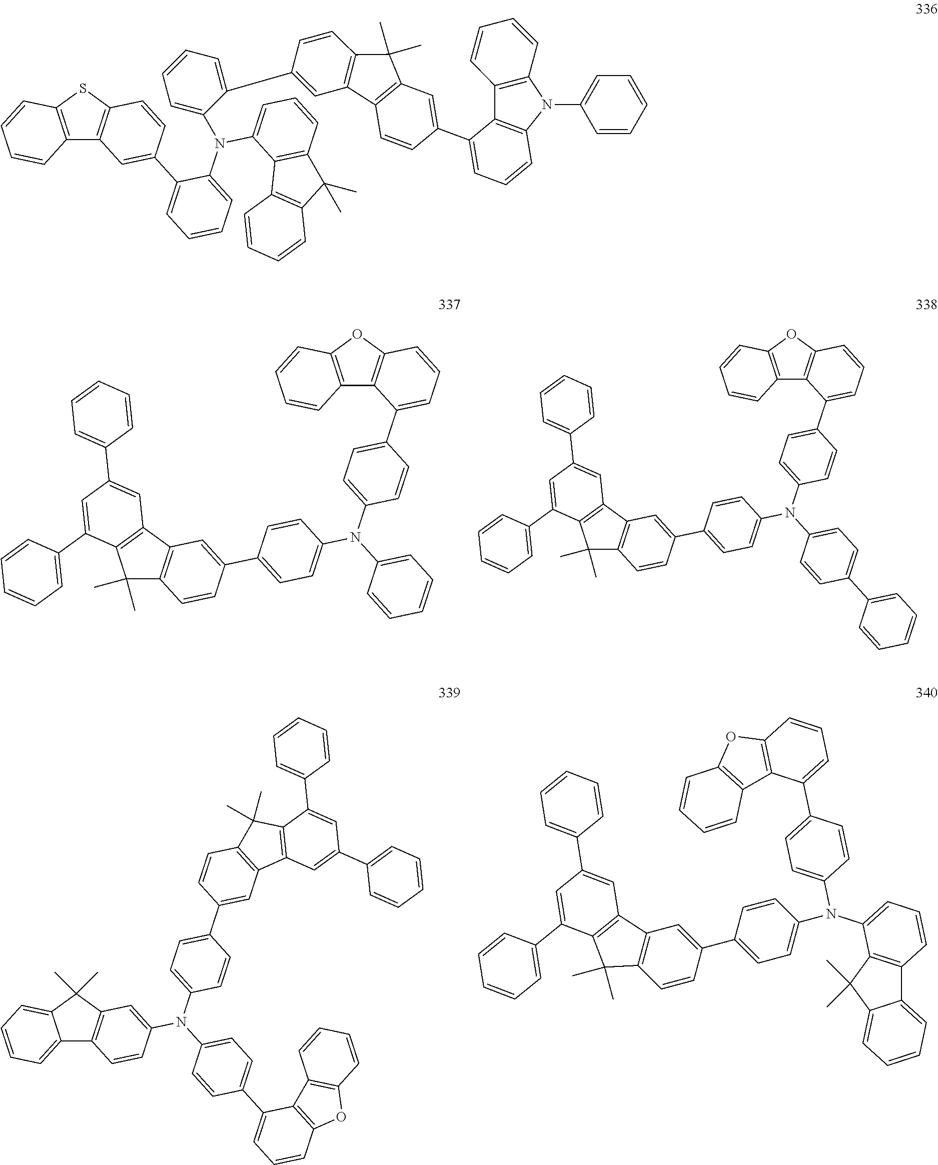

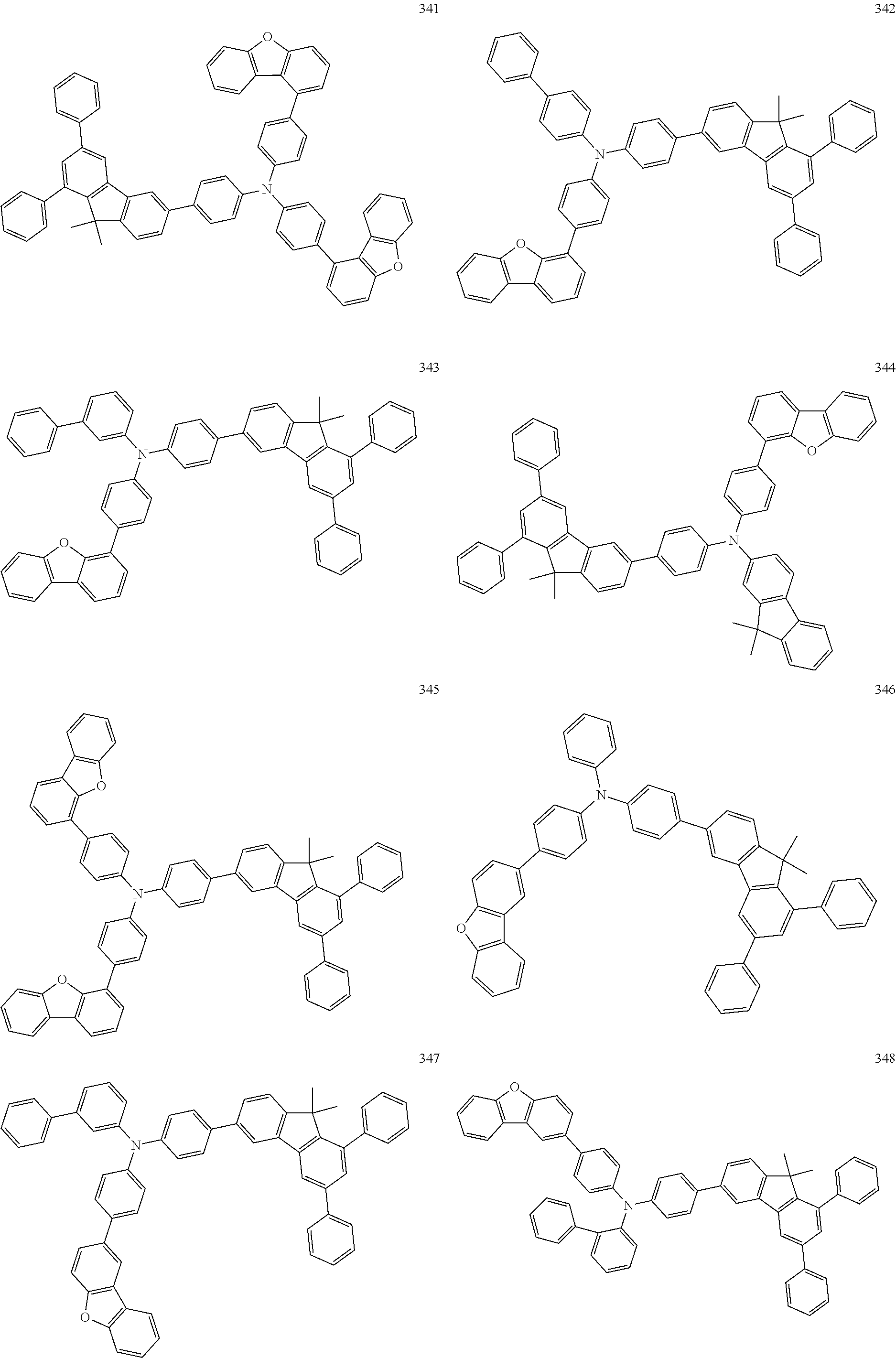

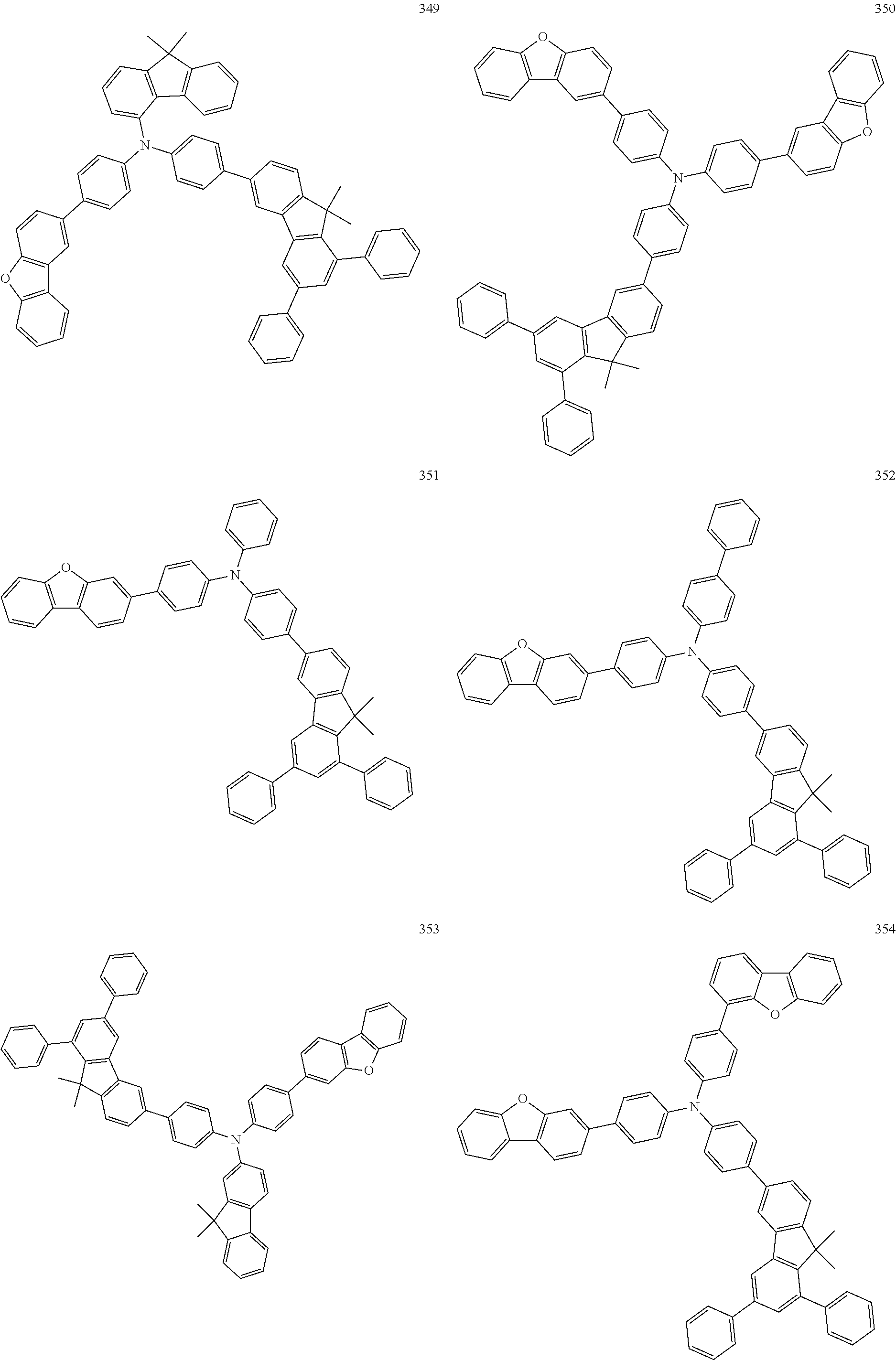

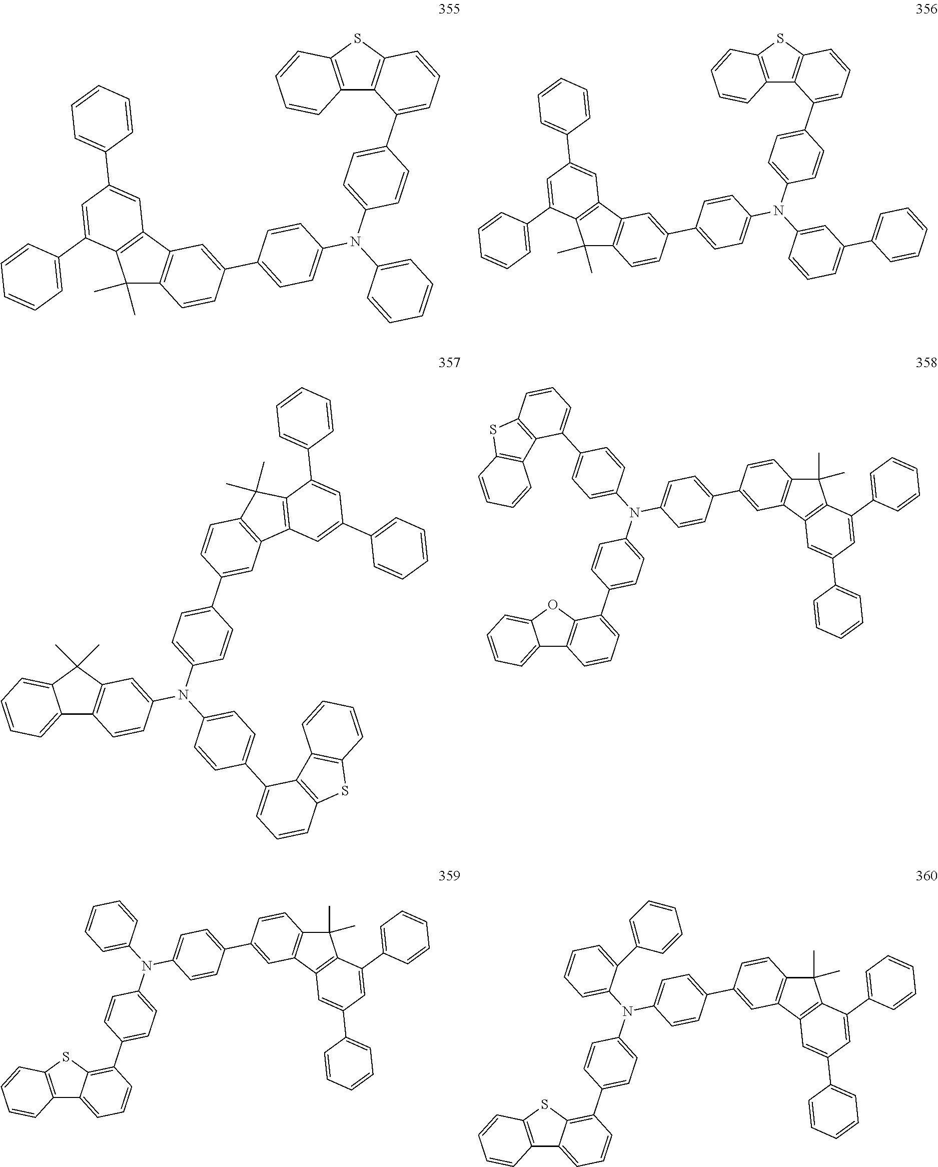

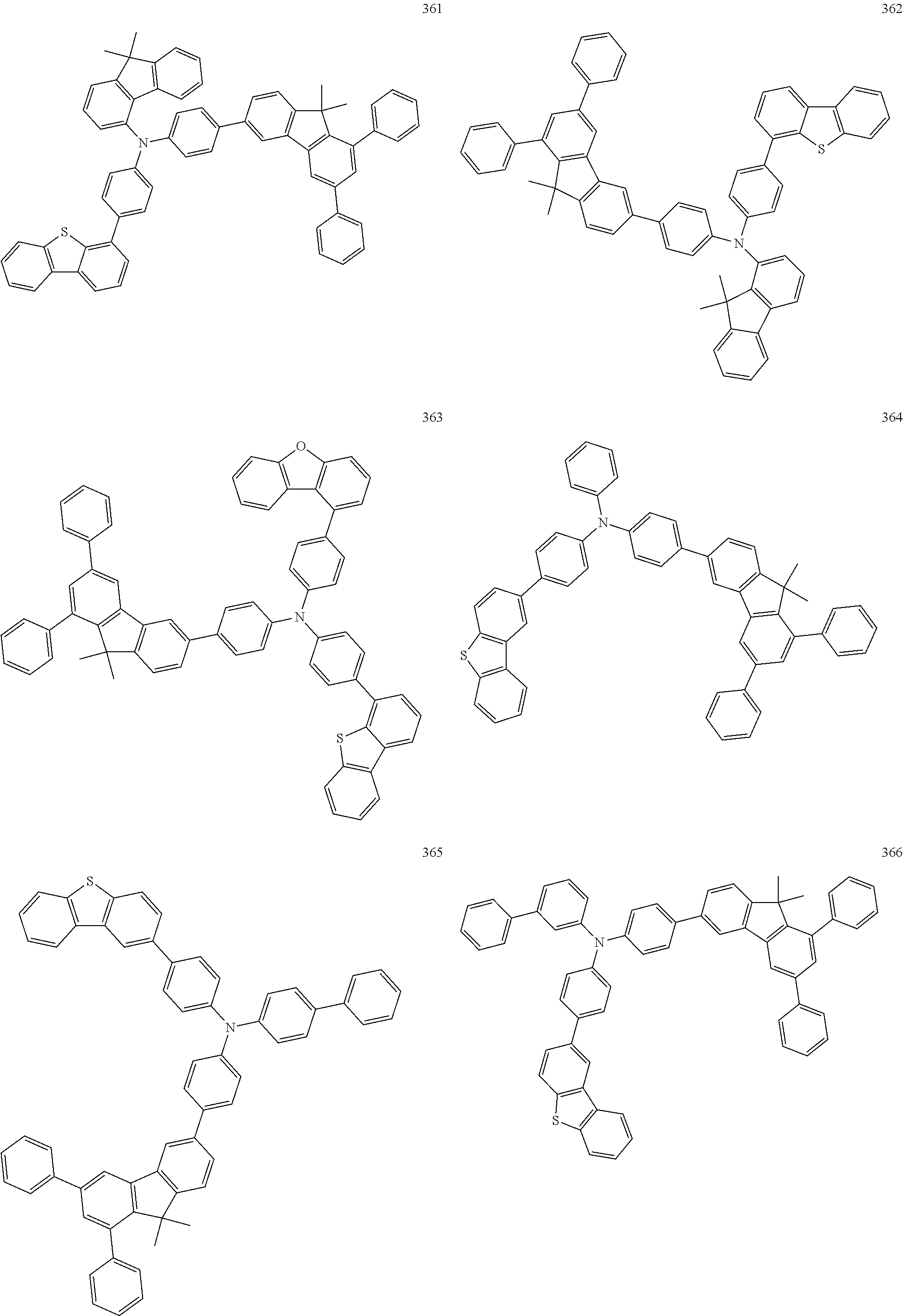

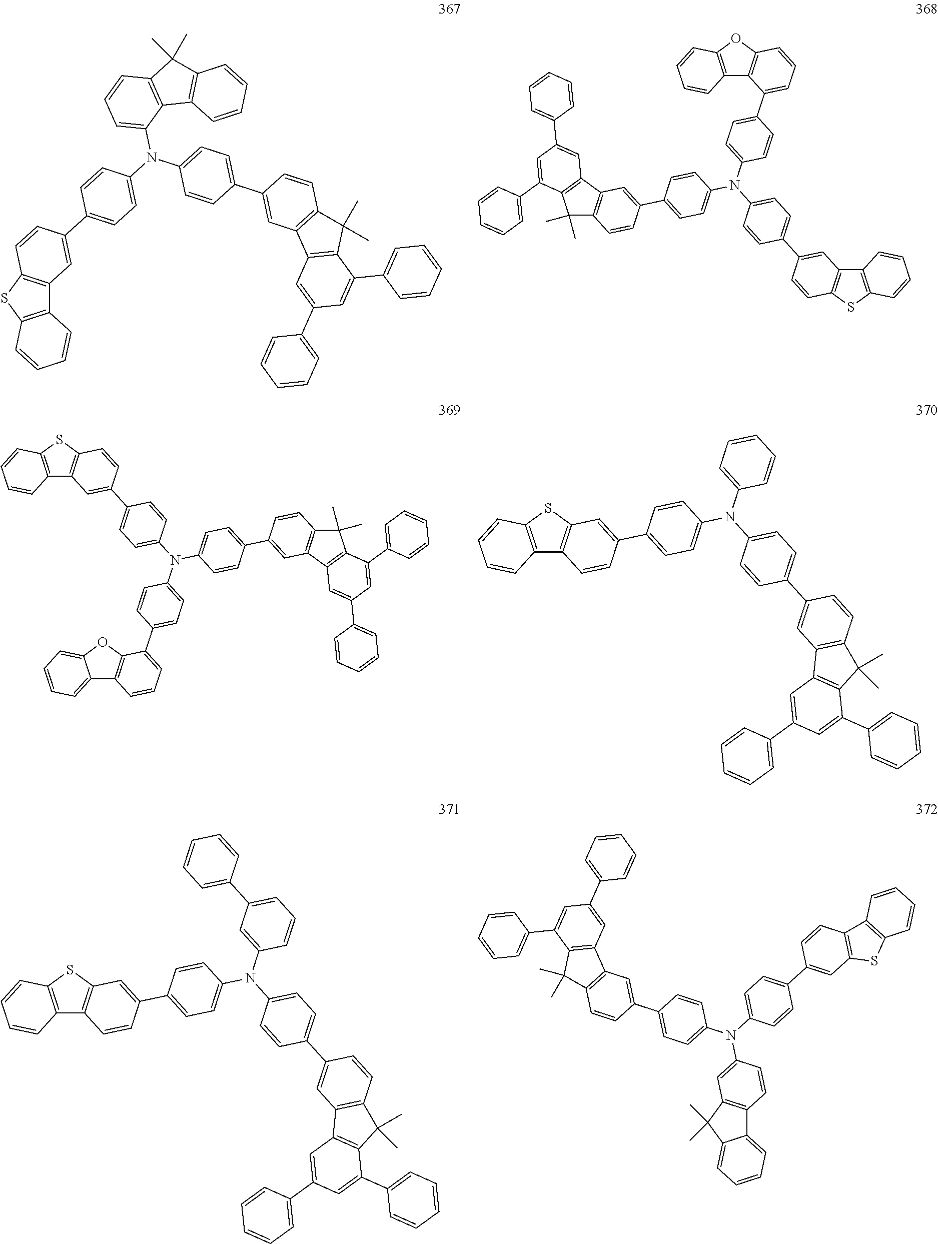

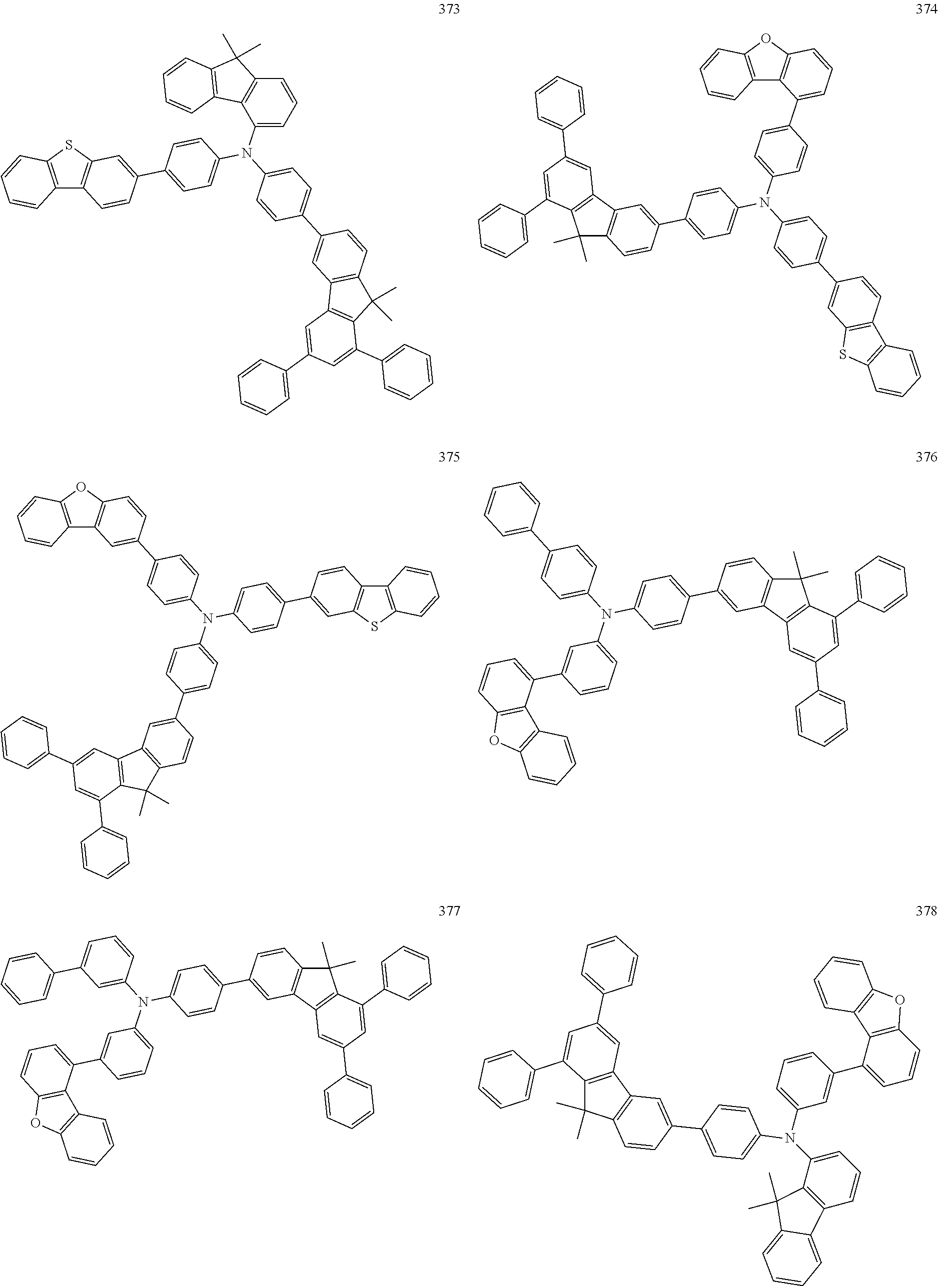

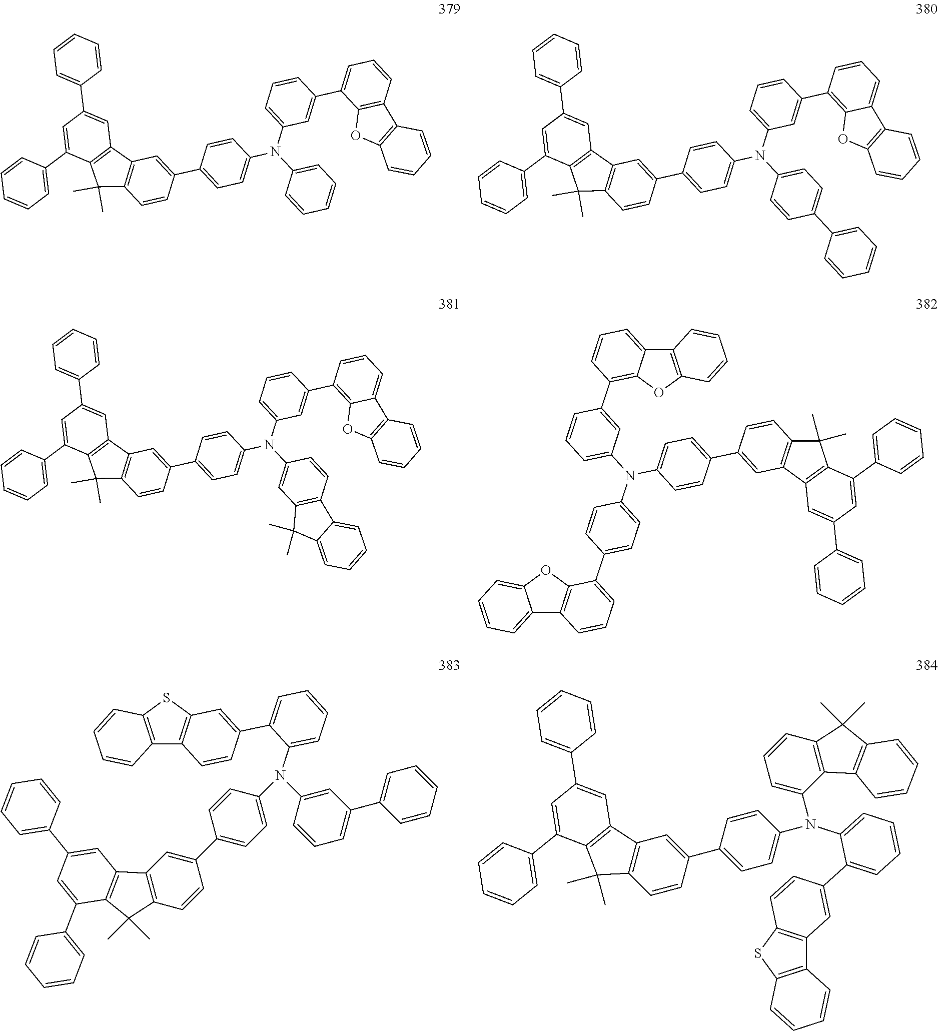

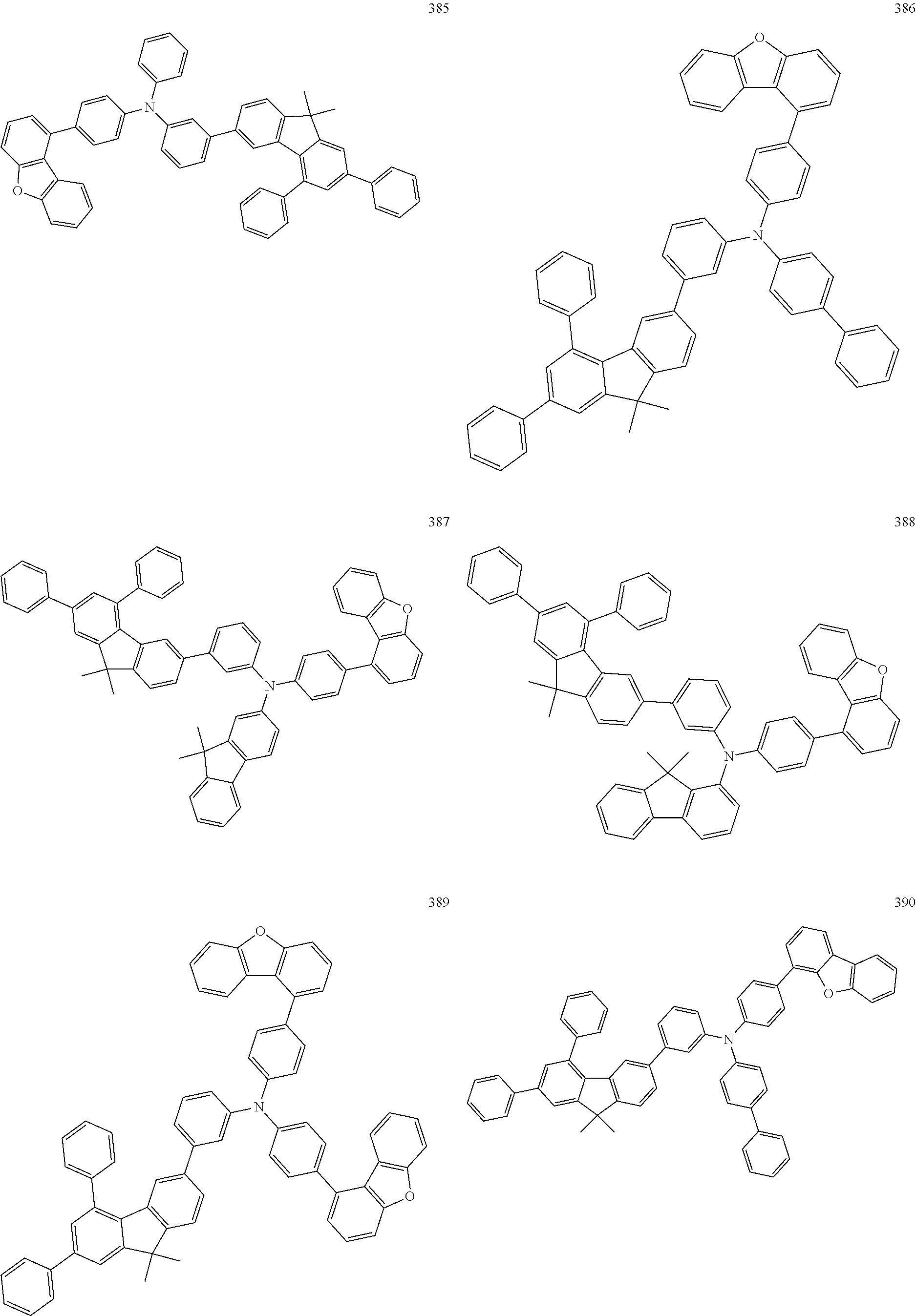

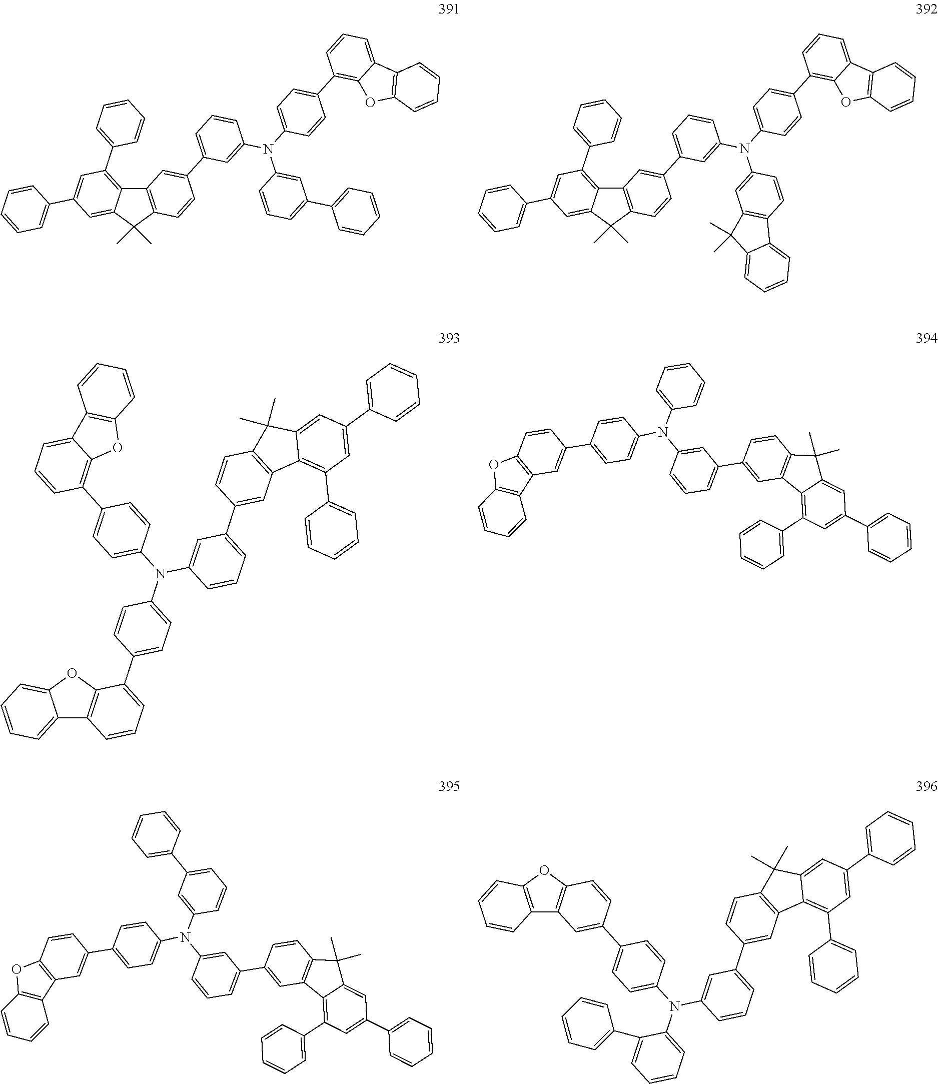

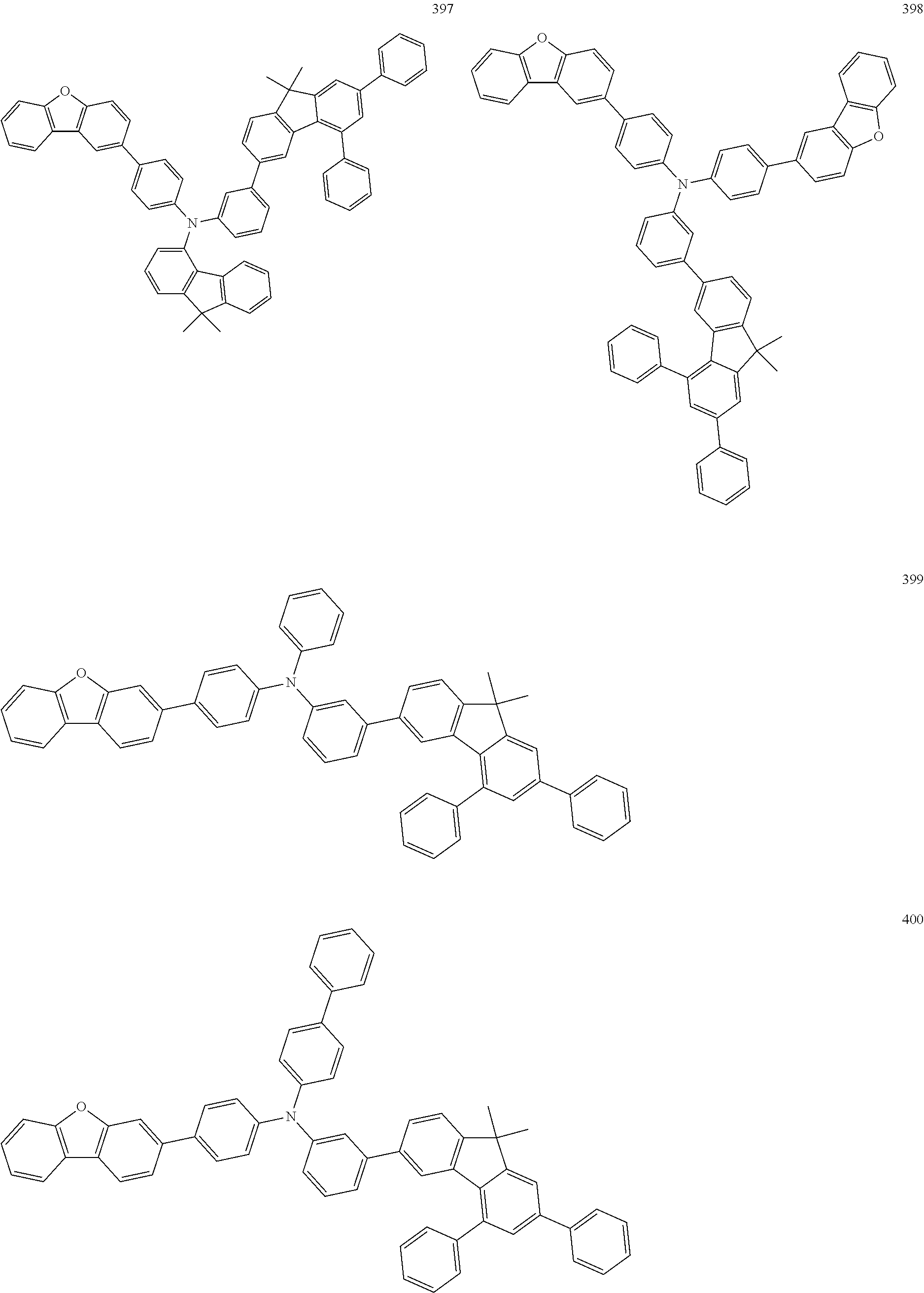 [SMILES: CC1(C)c2ccc(-c3cccc(N(c4ccc(-c5ccc6oc7ccccc7c6c5)cc4)c4cccc5c4-c4ccccc4C5(C)C)c3)cc2-c2c(-c3ccccc3)cc(-c3ccccc3)cc21 ; CC1(C)c2ccc(-c3cccc(N(c4ccc(-c5ccc6oc7ccccc7c6c5)cc4)c4ccc(-c5ccc6oc7ccccc7c6c5)cc4)c3)cc2-c2c(-c3ccccc3)cc(-c3ccccc3)cc21 ; CC1(C)c2ccc(-c3cccc(N(c4ccccc4)c4ccc(-c5ccc6c(c5)oc5ccccc56)cc4)c3)cc2-c2c(-c3ccccc3)cc(-c3ccccc3)cc21 ; CC1(C)c2ccc(-c3cccc(N(c4ccc(-c5ccccc5)cc4)c4ccc(-c5ccc6c(c5)oc5ccccc56)cc4)c3)cc2-c2c(-c3ccccc3)cc(-c3ccccc3)cc21]

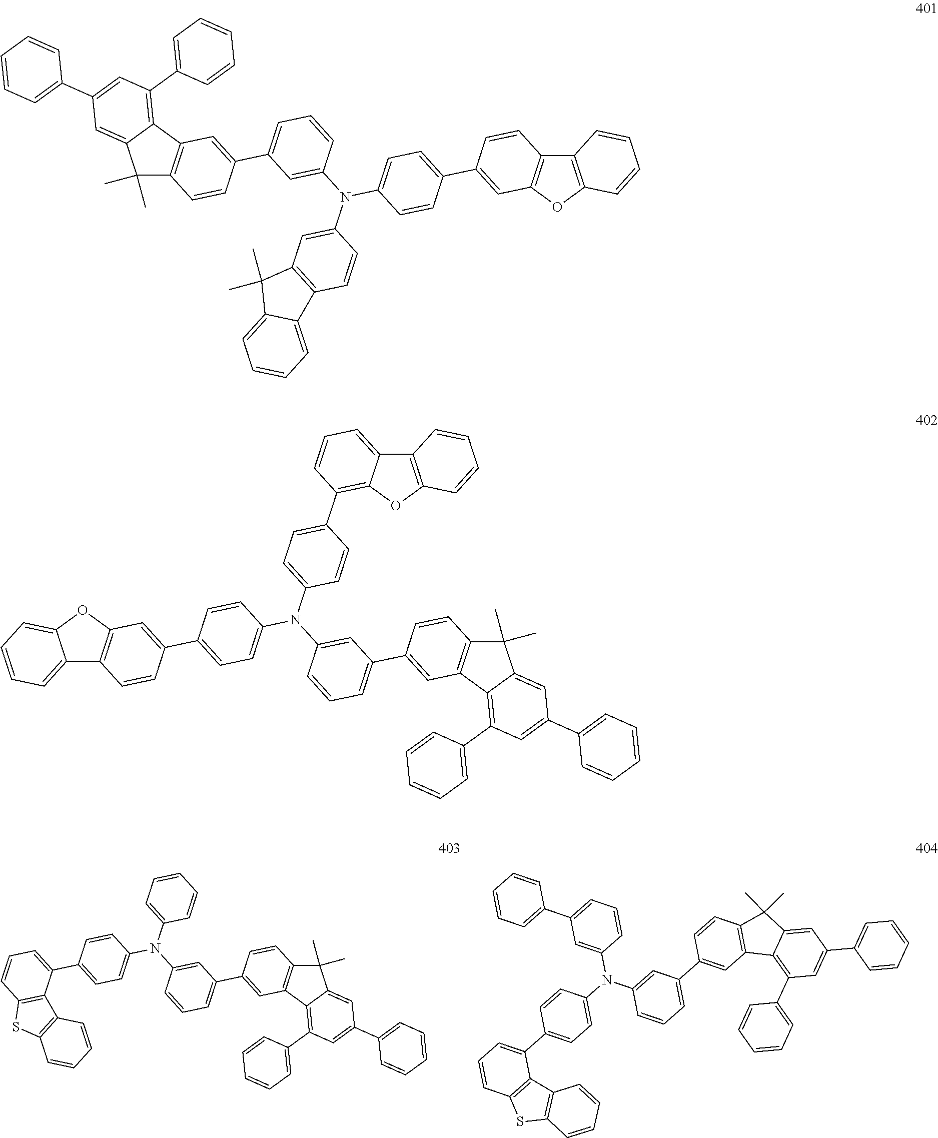 [SMILES: CC1(C)c2ccccc2-c2ccc(N(c3ccc(-c4ccc5c(c4)oc4ccccc45)cc3)c3cccc(-c4ccc5c(c4)-c4c(-c6ccccc6)cc(-c6ccccc6)cc4C5(C)C)c3)cc21 ; CC1(C)c2ccc(-c3cccc(N(c4ccc(-c5ccc6c(c5)oc5ccccc56)cc4)c4ccc(-c5cccc6c5oc5ccccc56)cc4)c3)cc2-c2c(-c3ccccc3)cc(-c3ccccc3)cc21 ; CC1(C)c2ccc(-c3cccc(N(c4ccccc4)c4ccc(-c5cccc6sc7ccccc7c56)cc4)c3)cc2-c2c(-c3ccccc3)cc(-c3ccccc3)cc21 ; CC1(C)c2ccc(-c3cccc(N(c4ccc(-c5cccc6sc7ccccc7c56)cc4)c4cccc(-c5ccccc5)c4)c3)cc2-c2c(-c3ccccc3)cc(-c3ccccc3)cc21]

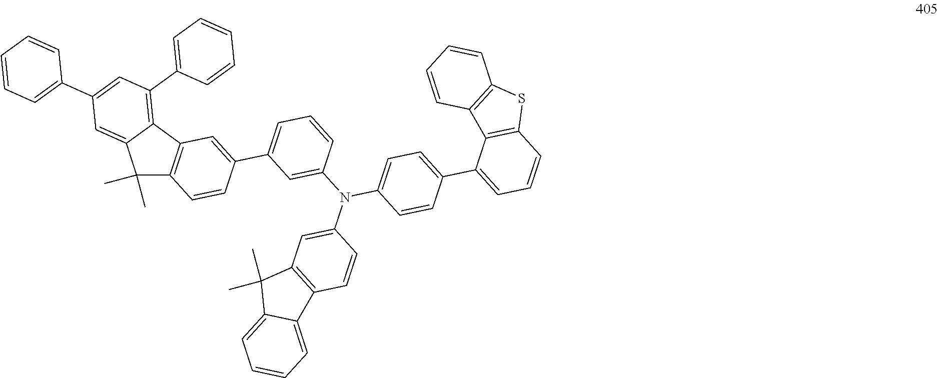 [SMILES: CC1(C)c2ccccc2-c2ccc(N(c3ccc(-c4cccc5sc6ccccc6c45)cc3)c3cccc(-c4ccc5c(c4)-c4c(-c6ccccc6)cc(-c6ccccc6)cc4C5(C)C)c3)cc21]

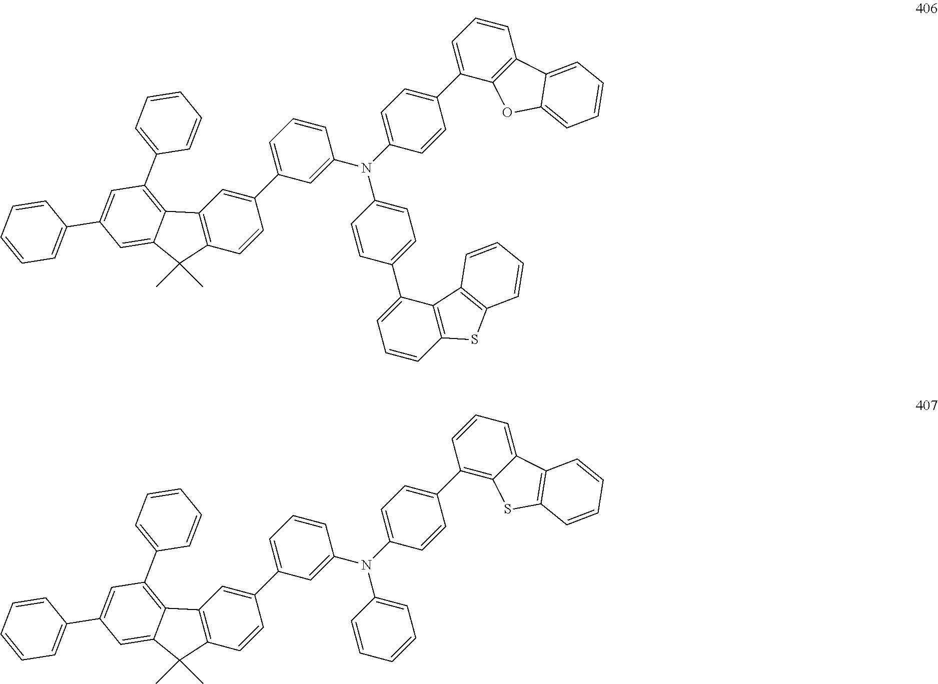 [SMILES: CC1(C)c2ccc(-c3cccc(N(c4ccc(-c5cccc6c5oc5ccccc56)cc4)c4ccc(-c5cccc6sc7ccccc7c56)cc4)c3)cc2-c2c(-c3ccccc3)cc(-c3ccccc3)cc21 ; CC1(C)c2ccc(-c3cccc(N(c4ccccc4)c4ccc(-c5cccc6c5sc5ccccc56)cc4)c3)cc2-c2c(-c3ccccc3)cc(-c3ccccc3)cc21]

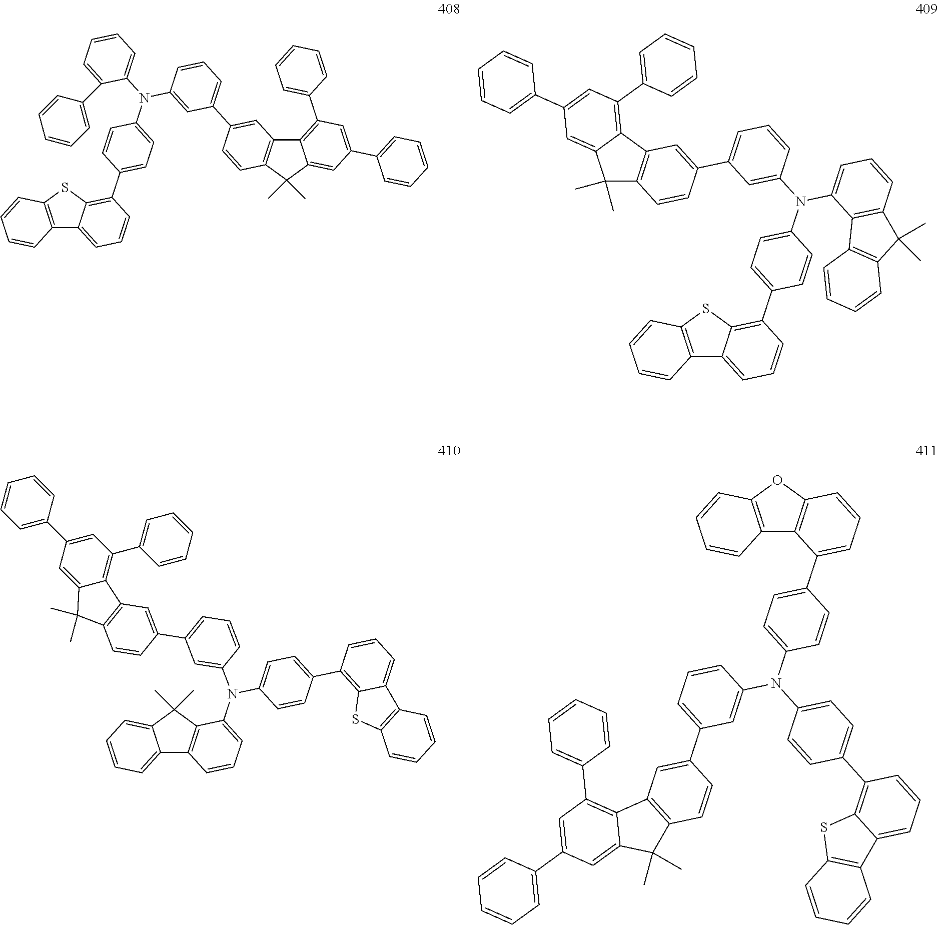 [SMILES: CC1(C)c2ccc(-c3cccc(N(c4ccc(-c5cccc6c5sc5ccccc56)cc4)c4ccccc4-c4ccccc4)c3)cc2-c2c(-c3ccccc3)cc(-c3ccccc3)cc21 ; CC1(C)c2ccc(-c3cccc(N(c4ccc(-c5cccc6c5sc5ccccc56)cc4)c4cccc5c4-c4ccccc4C5(C)C)c3)cc2-c2c(-c3ccccc3)cc(-c3ccccc3)cc21 ; CC1(C)c2ccc(-c3cccc(N(c4ccc(-c5cccc6c5sc5ccccc56)cc4)c4cccc5c4C(C)(C)c4ccccc4-5)c3)cc2-c2c(-c3ccccc3)cc(-c3ccccc3)cc21 ; CC1(C)c2ccc(-c3cccc(N(c4ccc(-c5cccc6c5sc5ccccc56)cc4)c4ccc(-c5cccc6oc7ccccc7c56)cc4)c3)cc2-c2c(-c3ccccc3)cc(-c3ccccc3)cc21]

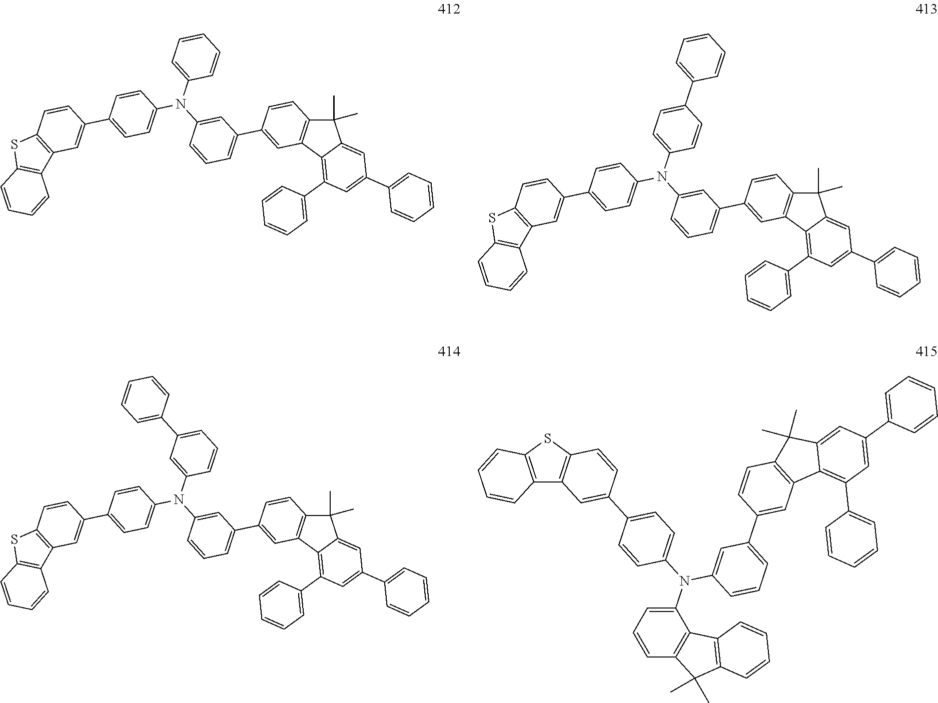 [SMILES: CC1(C)c2ccc(-c3cccc(N(c4ccccc4)c4ccc(-c5ccc6sc7ccccc7c6c5)cc4)c3)cc2-c2c(-c3ccccc3)cc(-c3ccccc3)cc21 ; CC1(C)c2ccc(-c3cccc(N(c4ccc(-c5ccccc5)cc4)c4ccc(-c5ccc6sc7ccccc7c6c5)cc4)c3)cc2-c2c(-c3ccccc3)cc(-c3ccccc3)cc21 ; CC1(C)c2ccc(-c3cccc(N(c4ccc(-c5ccc6sc7ccccc7c6c5)cc4)c4cccc(-c5ccccc5)c4)c3)cc2-c2c(-c3ccccc3)cc(-c3ccccc3)cc21 ; CC1(C)c2ccc(-c3cccc(N(c4ccc(-c5ccc6sc7ccccc7c6c5)cc4)c4cccc5c4-c4ccccc4C5(C)C)c3)cc2-c2c(-c3ccccc3)cc(-c3ccccc3)cc21]

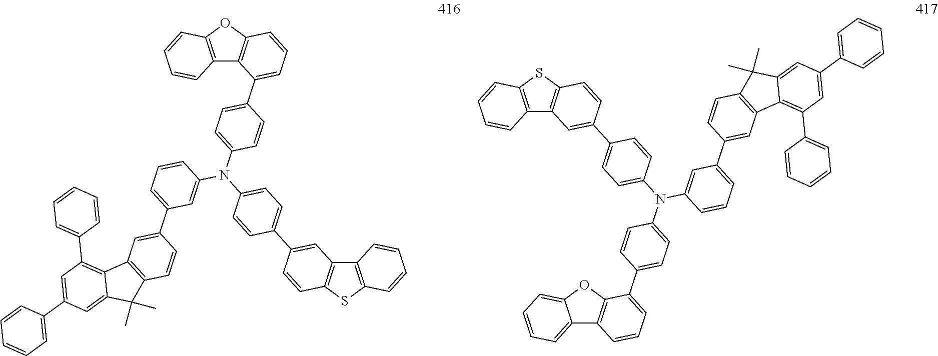 [SMILES: CC1(C)c2ccc(-c3cccc(N(c4ccc(-c5ccc6sc7ccccc7c6c5)cc4)c4ccc(-c5cccc6oc7ccccc7c56)cc4)c3)cc2-c2c(-c3ccccc3)cc(-c3ccccc3)cc21 ; CC1(C)c2ccc(-c3cccc(N(c4ccc(-c5ccc6sc7ccccc7c6c5)cc4)c4ccc(-c5cccc6c5oc5ccccc56)cc4)c3)cc2-c2c(-c3ccccc3)cc(-c3ccccc3)cc21]

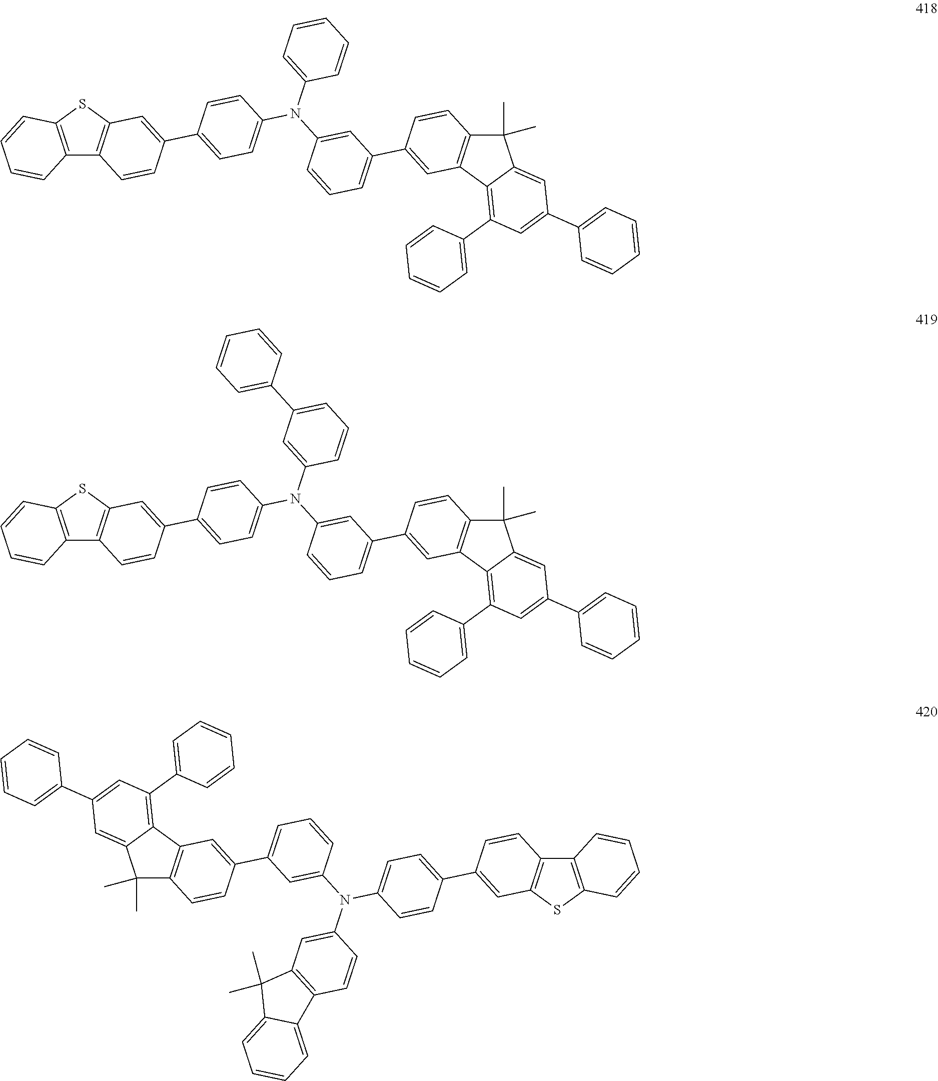 [SMILES: CC1(C)c2ccc(-c3cccc(N(c4ccccc4)c4ccc(-c5ccc6c(c5)sc5ccccc56)cc4)c3)cc2-c2c(-c3ccccc3)cc(-c3ccccc3)cc21 ; CC1(C)c2ccc(-c3cccc(N(c4ccc(-c5ccc6c(c5)sc5ccccc56)cc4)c4cccc(-c5ccccc5)c4)c3)cc2-c2c(-c3ccccc3)cc(-c3ccccc3)cc21 ; CC1(C)c2ccccc2-c2ccc(N(c3ccc(-c4ccc5c(c4)sc4ccccc45)cc3)c3cccc(-c4ccc5c(c4)-c4c(-c6ccccc6)cc(-c6ccccc6)cc4C5(C)C)c3)cc21]

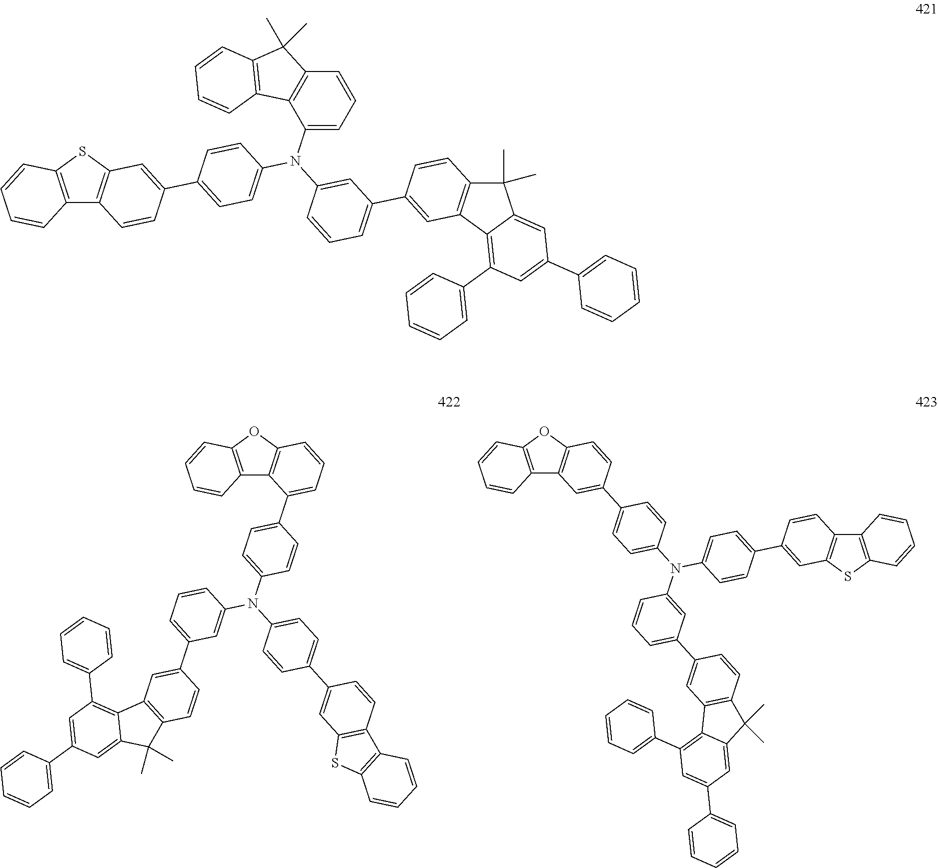 [SMILES: CC1(C)c2ccc(-c3cccc(N(c4ccc(-c5ccc6c(c5)sc5ccccc56)cc4)c4cccc5c4-c4ccccc4C5(C)C)c3)cc2-c2c(-c3ccccc3)cc(-c3ccccc3)cc21 ; CC1(C)c2ccc(-c3cccc(N(c4ccc(-c5ccc6c(c5)sc5ccccc56)cc4)c4ccc(-c5cccc6oc7ccccc7c56)cc4)c3)cc2-c2c(-c3ccccc3)cc(-c3ccccc3)cc21 ; CC1(C)c2ccc(-c3cccc(N(c4ccc(-c5ccc6c(c5)sc5ccccc56)cc4)c4ccc(-c5ccc6oc7ccccc7c6c5)cc4)c3)cc2-c2c(-c3ccccc3)cc(-c3ccccc3)cc21]

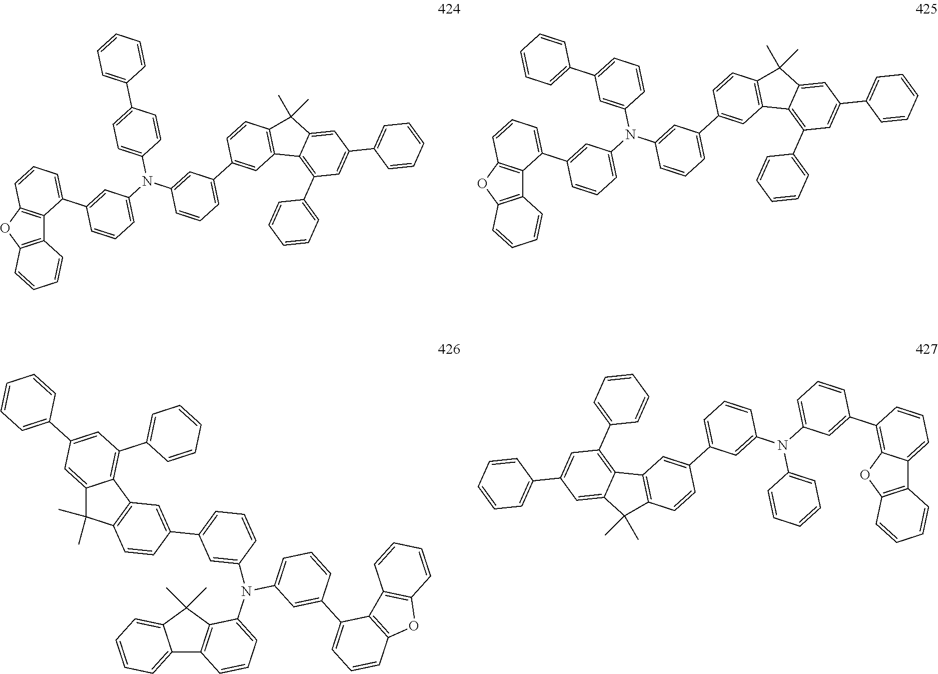 [SMILES: CC1(C)c2ccc(-c3cccc(N(c4ccc(-c5ccccc5)cc4)c4cccc(-c5cccc6oc7ccccc7c56)c4)c3)cc2-c2c(-c3ccccc3)cc(-c3ccccc3)cc21 ; CC1(C)c2ccc(-c3cccc(N(c4cccc(-c5ccccc5)c4)c4cccc(-c5cccc6oc7ccccc7c56)c4)c3)cc2-c2c(-c3ccccc3)cc(-c3ccccc3)cc21 ; CC1(C)c2ccc(-c3cccc(N(c4cccc(-c5cccc6oc7ccccc7c56)c4)c4cccc5c4C(C)(C)c4ccccc4-5)c3)cc2-c2c(-c3ccccc3)cc(-c3ccccc3)cc21 ; CC1(C)c2ccc(-c3cccc(N(c4ccccc4)c4cccc(-c5cccc6c5oc5ccccc56)c4)c3)cc2-c2c(-c3ccccc3)cc(-c3ccccc3)cc21]

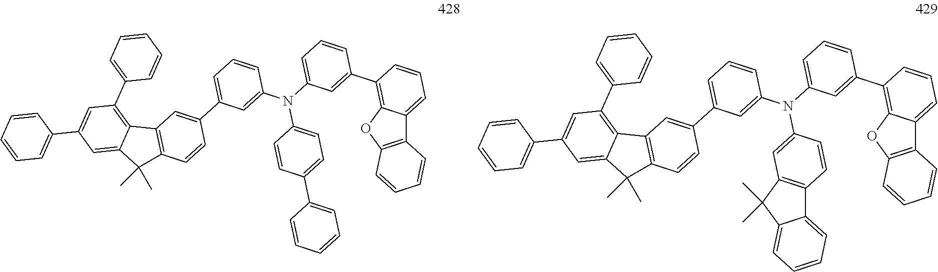 [SMILES: CC1(C)c2ccc(-c3cccc(N(c4ccc(-c5ccccc5)cc4)c4cccc(-c5cccc6c5oc5ccccc56)c4)c3)cc2-c2c(-c3ccccc3)cc(-c3ccccc3)cc21 ; CC1(C)c2ccccc2-c2ccc(N(c3cccc(-c4ccc5c(c4)-c4c(-c6ccccc6)cc(-c6ccccc6)cc4C5(C)C)c3)c3cccc(-c4cccc5c4oc4ccccc45)c3)cc21]

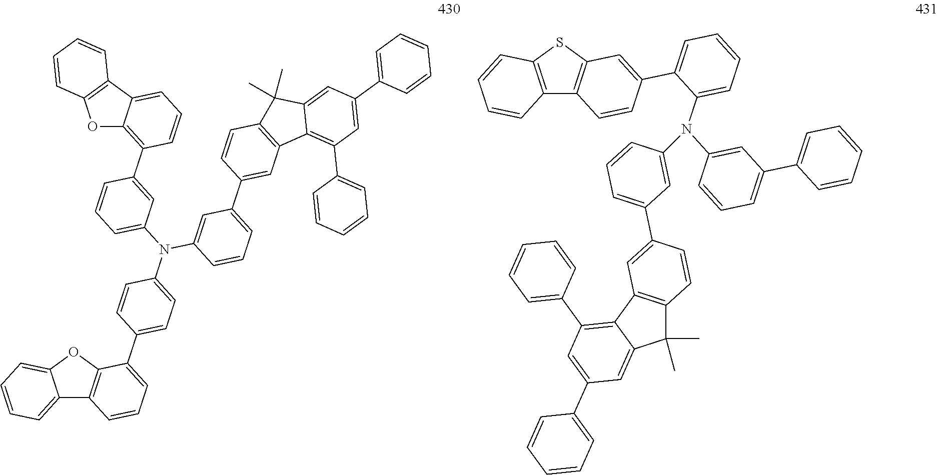 [SMILES: CC1(C)c2ccc(-c3cccc(N(c4ccc(-c5cccc6c5oc5ccccc56)cc4)c4cccc(-c5cccc6c5oc5ccccc56)c4)c3)cc2-c2c(-c3ccccc3)cc(-c3ccccc3)cc21 ; CC1(C)c2ccc(-c3cccc(N(c4cccc(-c5ccccc5)c4)c4ccccc4-c4ccc5c(c4)sc4ccccc45)c3)cc2-c2c(-c3ccccc3)cc(-c3ccccc3)cc21]

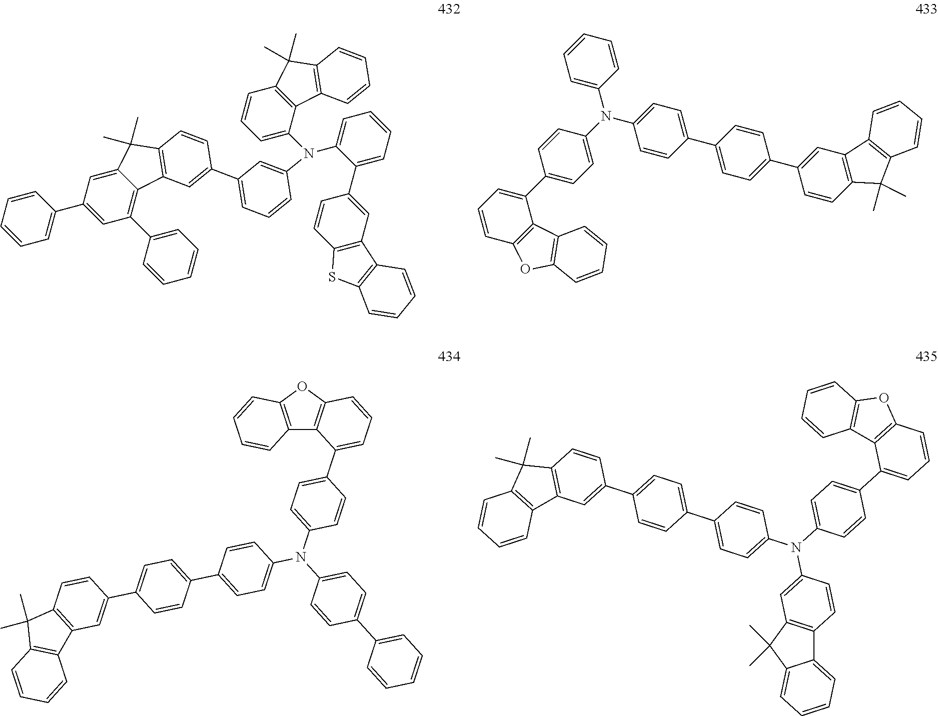 [SMILES: CC1(C)c2ccc(-c3cccc(N(c4ccccc4-c4ccc5sc6ccccc6c5c4)c4cccc5c4-c4ccccc4C5(C)C)c3)cc2-c2c(-c3ccccc3)cc(-c3ccccc3)cc21 ; CC1(C)c2ccccc2-c2cc(-c3ccc(-c4ccc(N(c5ccccc5)c5ccc(-c6cccc7oc8ccccc8c67)cc5)cc4)cc3)ccc21 ; CC1(C)c2ccccc2-c2cc(-c3ccc(-c4ccc(N(c5ccc(-c6ccccc6)cc5)c5ccc(-c6cccc7oc8ccccc8c67)cc5)cc4)cc3)ccc21 ; CC1(C)c2ccccc2-c2cc(-c3ccc(-c4ccc(N(c5ccc(-c6cccc7oc8ccccc8c67)cc5)c5ccc6c(c5)C(C)(C)c5ccccc5-6)cc4)cc3)ccc21]

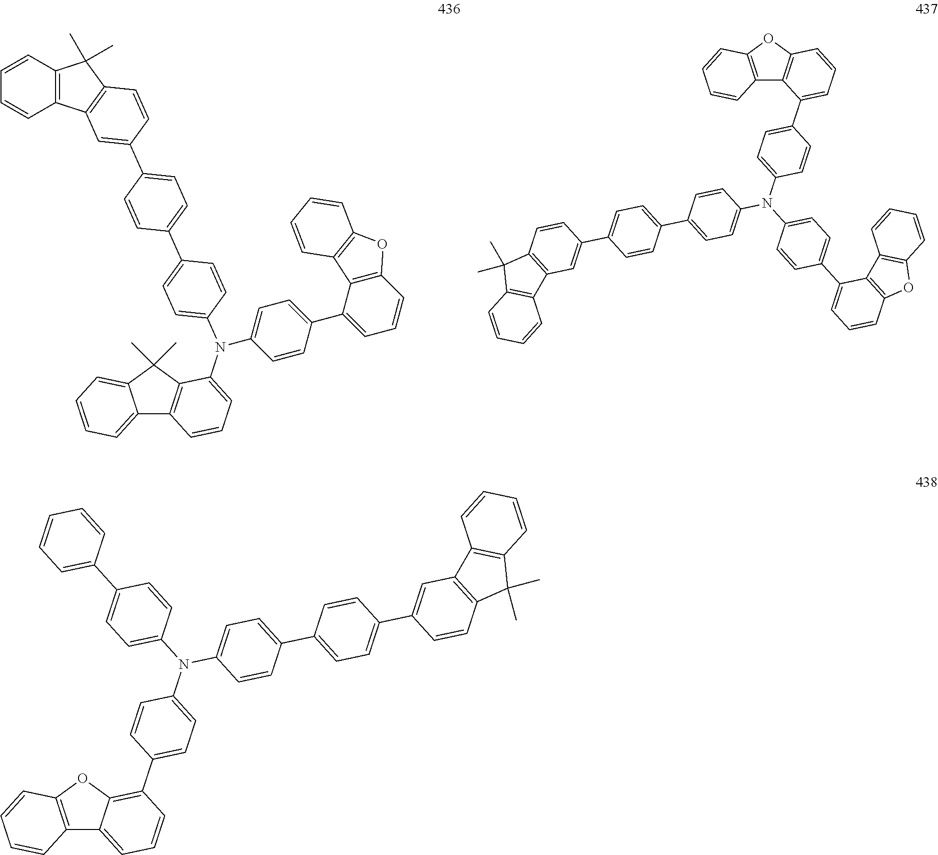 [SMILES: CC1(C)c2ccccc2-c2cc(-c3ccc(-c4ccc(N(c5ccc(-c6cccc7oc8ccccc8c67)cc5)c5cccc6c5C(C)(C)c5ccccc5-6)cc4)cc3)ccc21 ; CC1(C)c2ccccc2-c2cc(-c3ccc(-c4ccc(N(c5ccc(-c6cccc7oc8ccccc8c67)cc5)c5ccc(-c6cccc7oc8ccccc8c67)cc5)cc4)cc3)ccc21 ; CC1(C)c2ccccc2-c2cc(-c3ccc(-c4ccc(N(c5ccc(-c6ccccc6)cc5)c5ccc(-c6cccc7c6oc6ccccc67)cc5)cc4)cc3)ccc21]

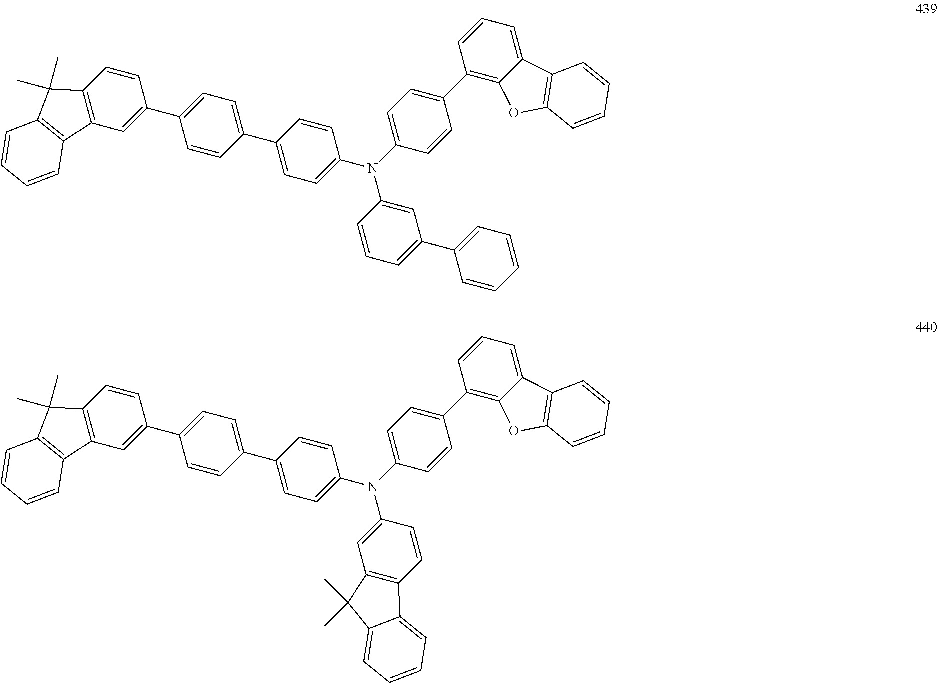 [SMILES: CC1(C)c2ccccc2-c2cc(-c3ccc(-c4ccc(N(c5ccc(-c6cccc7c6oc6ccccc67)cc5)c5cccc(-c6ccccc6)c5)cc4)cc3)ccc21 ; CC1(C)c2ccccc2-c2cc(-c3ccc(-c4ccc(N(c5ccc(-c6cccc7c6oc6ccccc67)cc5)c5ccc6c(c5)C(C)(C)c5ccccc5-6)cc4)cc3)ccc21]

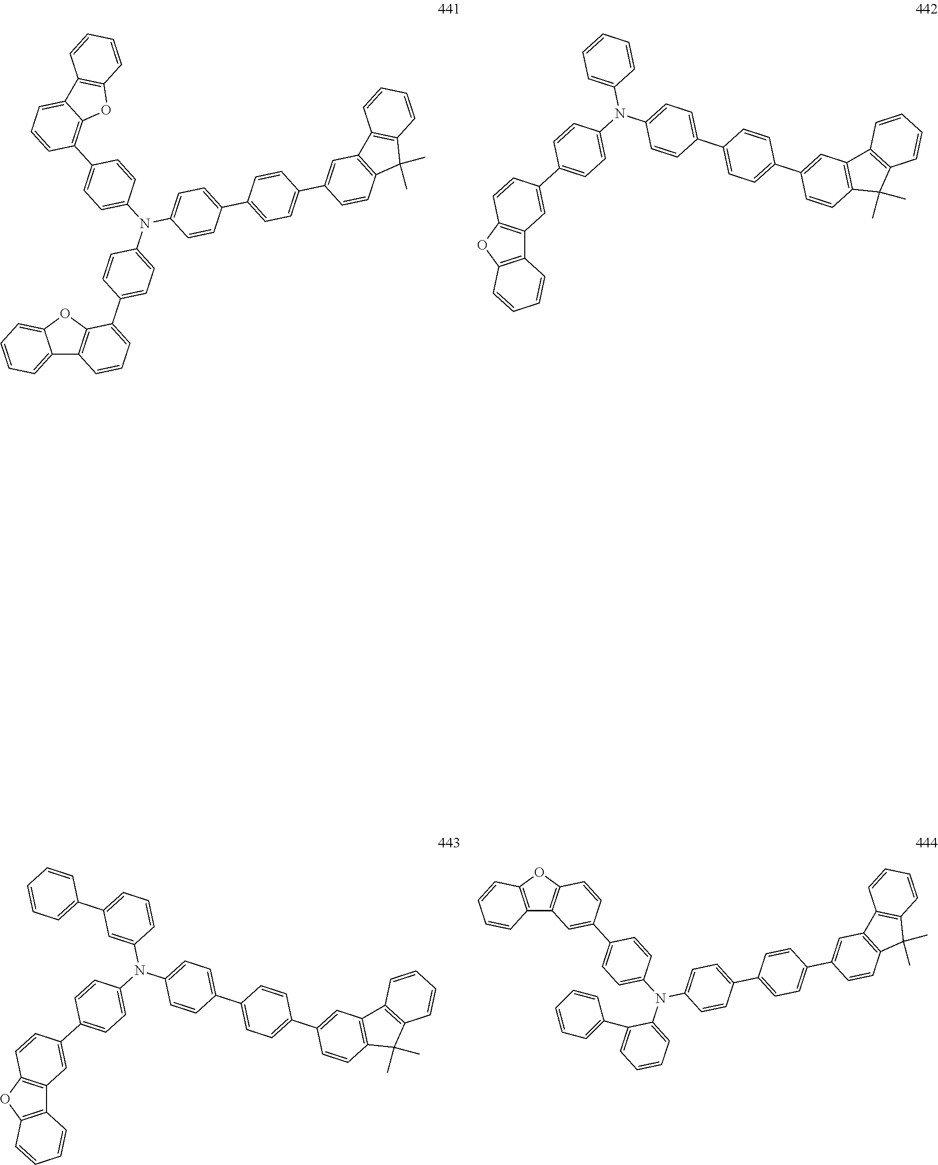 [SMILES: CC1(C)c2ccccc2-c2cc(-c3ccc(-c4ccc(N(c5ccc(-c6cccc7c6oc6ccccc67)cc5)c5ccc(-c6cccc7c6oc6ccccc67)cc5)cc4)cc3)ccc21 ; CC1(C)c2ccccc2-c2cc(-c3ccc(-c4ccc(N(c5ccccc5)c5ccc(-c6ccc7oc8ccccc8c7c6)cc5)cc4)cc3)ccc21 ; CC1(C)c2ccccc2-c2cc(-c3ccc(-c4ccc(N(c5ccc(-c6ccc7oc8ccccc8c7c6)cc5)c5cccc(-c6ccccc6)c5)cc4)cc3)ccc21 ; CC1(C)c2ccccc2-c2cc(-c3ccc(-c4ccc(N(c5ccc(-c6ccc7oc8ccccc8c7c6)cc5)c5ccccc5-c5ccccc5)cc4)cc3)ccc21]

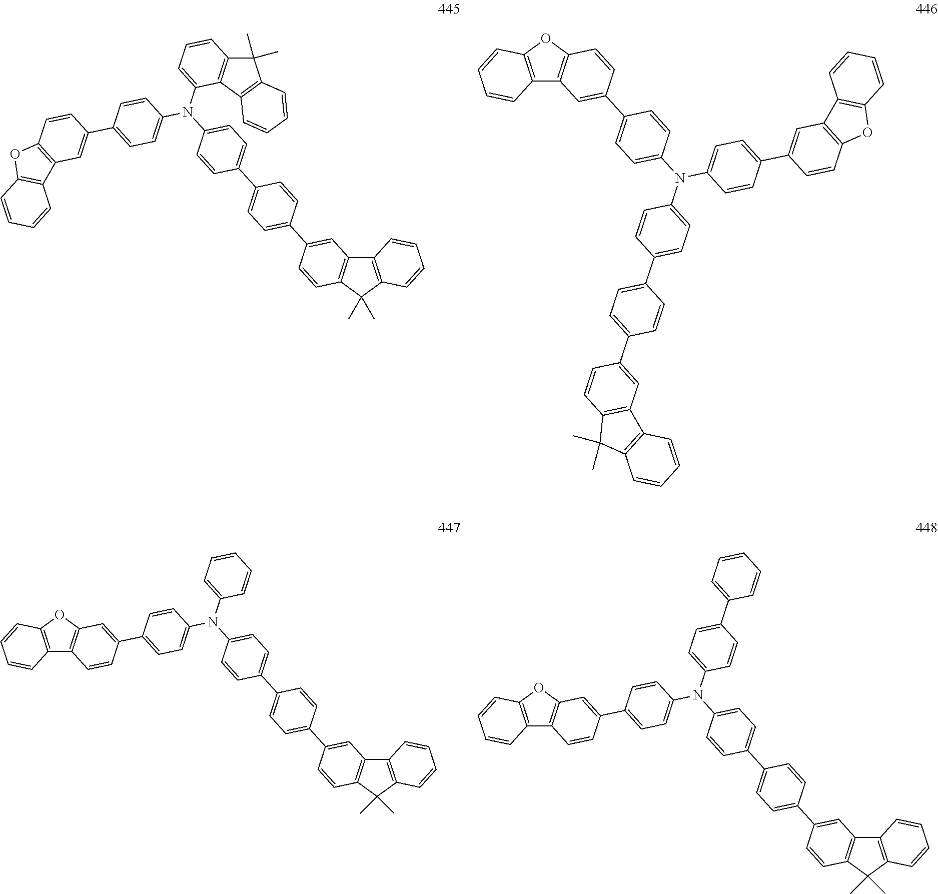 [SMILES: CC1(C)c2ccccc2-c2cc(-c3ccc(-c4ccc(N(c5ccc(-c6ccc7oc8ccccc8c7c6)cc5)c5cccc6c5-c5ccccc5C6(C)C)cc4)cc3)ccc21 ; CC1(C)c2ccccc2-c2cc(-c3ccc(-c4ccc(N(c5ccc(-c6ccc7oc8ccccc8c7c6)cc5)c5ccc(-c6ccc7oc8ccccc8c7c6)cc5)cc4)cc3)ccc21 ; CC1(C)c2ccccc2-c2cc(-c3ccc(-c4ccc(N(c5ccccc5)c5ccc(-c6ccc7c(c6)oc6ccccc67)cc5)cc4)cc3)ccc21 ; CC1(C)c2ccccc2-c2cc(-c3ccc(-c4ccc(N(c5ccc(-c6ccccc6)cc5)c5ccc(-c6ccc7c(c6)oc6ccccc67)cc5)cc4)cc3)ccc21]

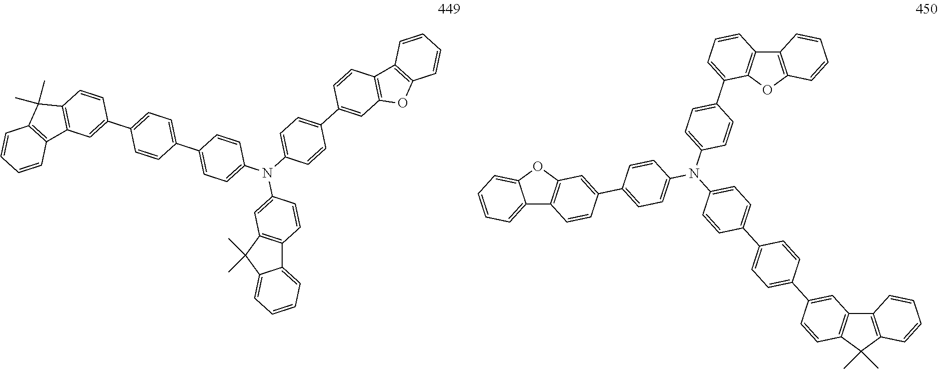 [SMILES: CC1(C)c2ccccc2-c2cc(-c3ccc(-c4ccc(N(c5ccc(-c6ccc7c(c6)oc6ccccc67)cc5)c5ccc6c(c5)C(C)(C)c5ccccc5-6)cc4)cc3)ccc21 ; CC1(C)c2ccccc2-c2cc(-c3ccc(-c4ccc(N(c5ccc(-c6ccc7c(c6)oc6ccccc67)cc5)c5ccc(-c6cccc7c6oc6ccccc67)cc5)cc4)cc3)ccc21]

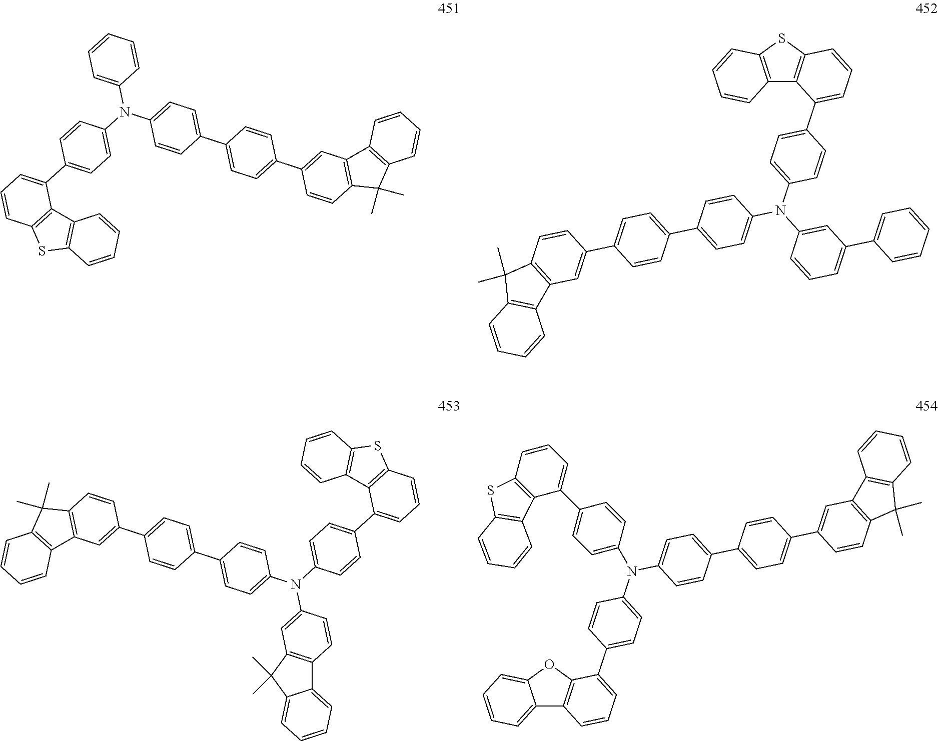 [SMILES: CC1(C)c2ccccc2-c2cc(-c3ccc(-c4ccc(N(c5ccccc5)c5ccc(-c6cccc7sc8ccccc8c67)cc5)cc4)cc3)ccc21 ; CC1(C)c2ccccc2-c2cc(-c3ccc(-c4ccc(N(c5ccc(-c6cccc7sc8ccccc8c67)cc5)c5cccc(-c6ccccc6)c5)cc4)cc3)ccc21 ; CC1(C)c2ccccc2-c2cc(-c3ccc(-c4ccc(N(c5ccc(-c6cccc7sc8ccccc8c67)cc5)c5ccc6c(c5)C(C)(C)c5ccccc5-6)cc4)cc3)ccc21 ; CC1(C)c2ccccc2-c2cc(-c3ccc(-c4ccc(N(c5ccc(-c6cccc7c6oc6ccccc67)cc5)c5ccc(-c6cccc7sc8ccccc8c67)cc5)cc4)cc3)ccc21]

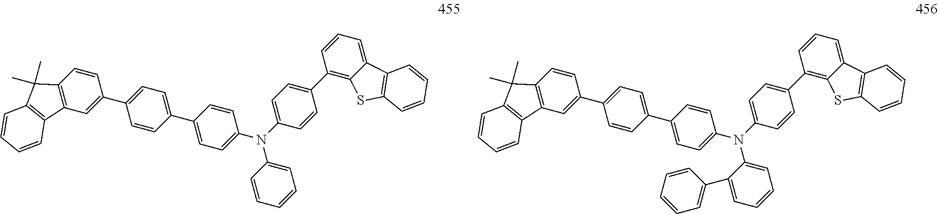 [SMILES: CC1(C)c2ccccc2-c2cc(-c3ccc(-c4ccc(N(c5ccccc5)c5ccc(-c6cccc7c6sc6ccccc67)cc5)cc4)cc3)ccc21 ; CC1(C)c2ccccc2-c2cc(-c3ccc(-c4ccc(N(c5ccc(-c6cccc7c6sc6ccccc67)cc5)c5ccccc5-c5ccccc5)cc4)cc3)ccc21]

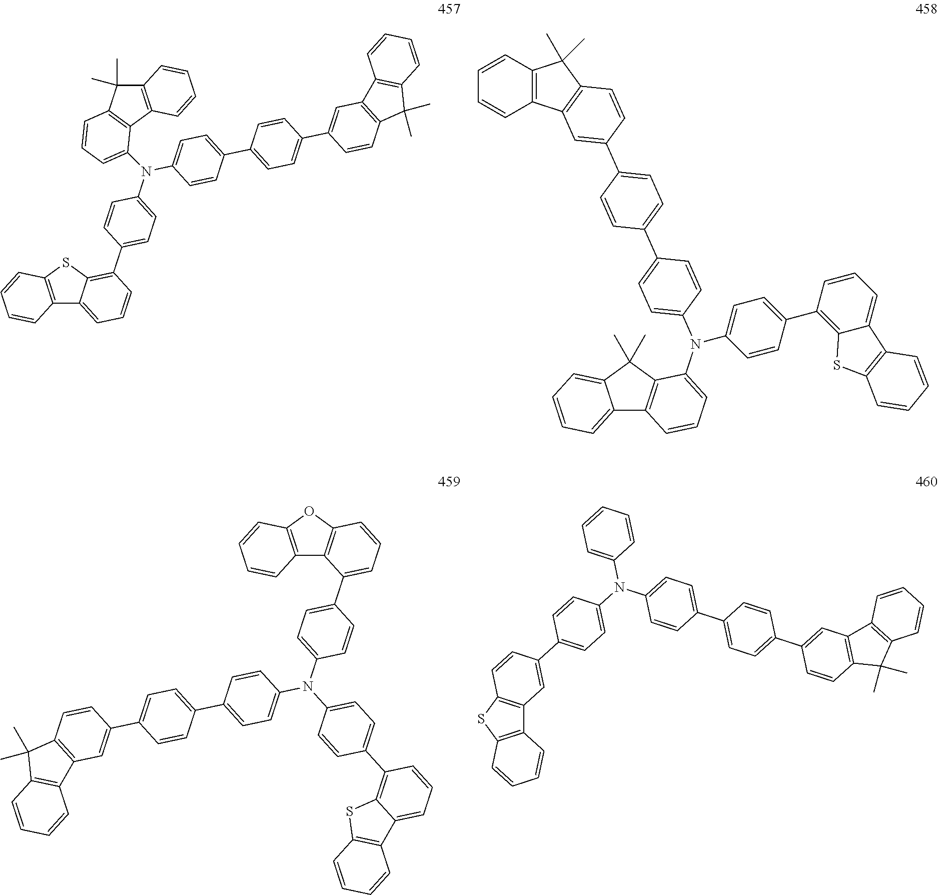 [SMILES: CC1(C)c2ccccc2-c2cc(-c3ccc(-c4ccc(N(c5ccc(-c6cccc7c6sc6ccccc67)cc5)c5cccc6c5-c5ccccc5C6(C)C)cc4)cc3)ccc21 ; CC1(C)c2ccccc2-c2cc(-c3ccc(-c4ccc(N(c5ccc(-c6cccc7c6sc6ccccc67)cc5)c5cccc6c5C(C)(C)c5ccccc5-6)cc4)cc3)ccc21 ; CC1(C)c2ccccc2-c2cc(-c3ccc(-c4ccc(N(c5ccc(-c6cccc7c6sc6ccccc67)cc5)c5ccc(-c6cccc7oc8ccccc8c67)cc5)cc4)cc3)ccc21 ; CC1(C)c2ccccc2-c2cc(-c3ccc(-c4ccc(N(c5ccccc5)c5ccc(-c6ccc7sc8ccccc8c7c6)cc5)cc4)cc3)ccc21]

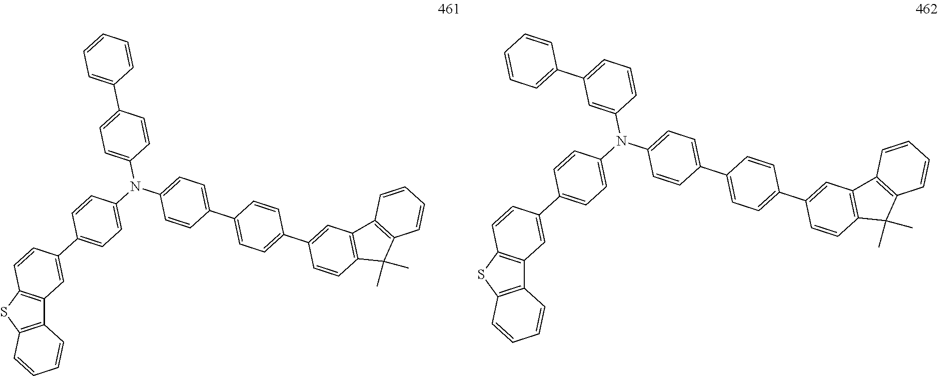 [SMILES: CC1(C)c2ccccc2-c2cc(-c3ccc(-c4ccc(N(c5ccc(-c6ccccc6)cc5)c5ccc(-c6ccc7sc8ccccc8c7c6)cc5)cc4)cc3)ccc21 ; CC1(C)c2ccccc2-c2cc(-c3ccc(-c4ccc(N(c5ccc(-c6ccc7sc8ccccc8c7c6)cc5)c5cccc(-c6ccccc6)c5)cc4)cc3)ccc21]

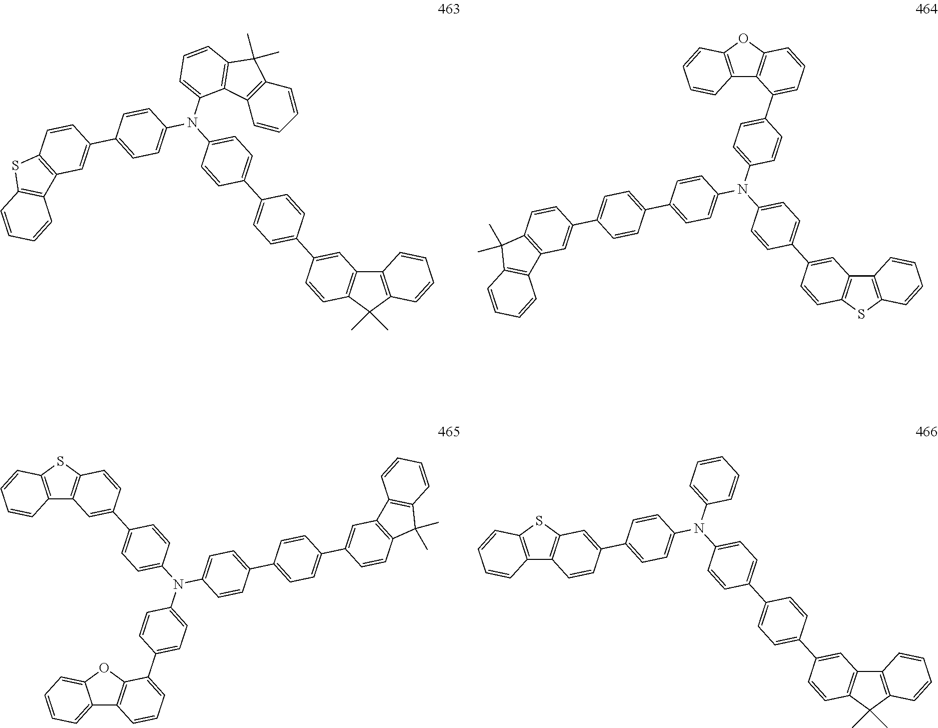 [SMILES: CC1(C)c2ccccc2-c2cc(-c3ccc(-c4ccc(N(c5ccc(-c6ccc7sc8ccccc8c7c6)cc5)c5cccc6c5-c5ccccc5C6(C)C)cc4)cc3)ccc21 ; CC1(C)c2ccccc2-c2cc(-c3ccc(-c4ccc(N(c5ccc(-c6ccc7sc8ccccc8c7c6)cc5)c5ccc(-c6cccc7oc8ccccc8c67)cc5)cc4)cc3)ccc21 ; CC1(C)c2ccccc2-c2cc(-c3ccc(-c4ccc(N(c5ccc(-c6ccc7sc8ccccc8c7c6)cc5)c5ccc(-c6cccc7c6oc6ccccc67)cc5)cc4)cc3)ccc21 ; CC1(C)c2ccccc2-c2cc(-c3ccc(-c4ccc(N(c5ccccc5)c5ccc(-c6ccc7c(c6)sc6ccccc67)cc5)cc4)cc3)ccc21]

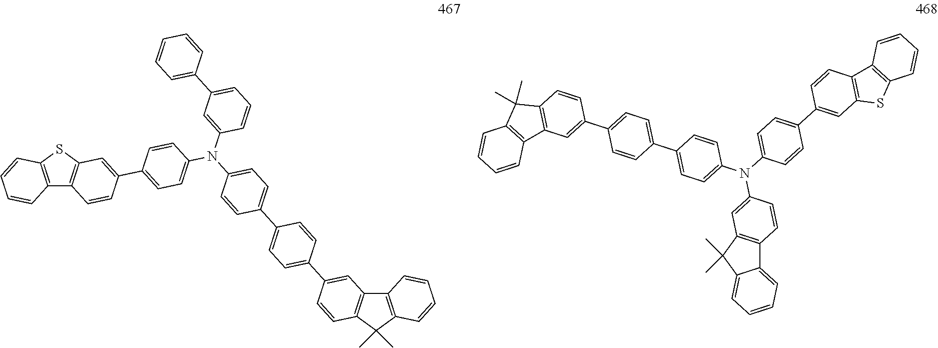 [SMILES: CC1(C)c2ccccc2-c2cc(-c3ccc(-c4ccc(N(c5ccc(-c6ccc7c(c6)sc6ccccc67)cc5)c5cccc(-c6ccccc6)c5)cc4)cc3)ccc21 ; CC1(C)c2ccccc2-c2cc(-c3ccc(-c4ccc(N(c5ccc(-c6ccc7c(c6)sc6ccccc67)cc5)c5ccc6c(c5)C(C)(C)c5ccccc5-6)cc4)cc3)ccc21]

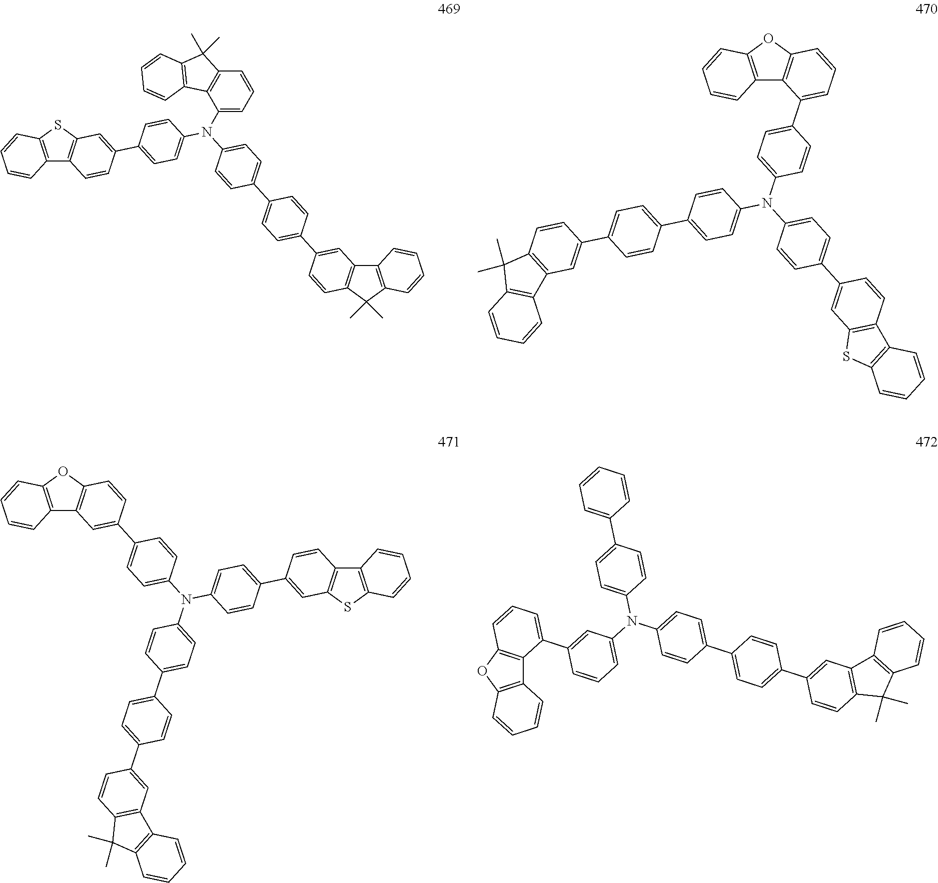 [SMILES: CC1(C)c2ccccc2-c2cc(-c3ccc(-c4ccc(N(c5ccc(-c6ccc7c(c6)sc6ccccc67)cc5)c5cccc6c5-c5ccccc5C6(C)C)cc4)cc3)ccc21 ; CC1(C)c2ccccc2-c2cc(-c3ccc(-c4ccc(N(c5ccc(-c6ccc7c(c6)sc6ccccc67)cc5)c5ccc(-c6cccc7oc8ccccc8c67)cc5)cc4)cc3)ccc21 ; CC1(C)c2ccccc2-c2cc(-c3ccc(-c4ccc(N(c5ccc(-c6ccc7c(c6)sc6ccccc67)cc5)c5ccc(-c6ccc7oc8ccccc8c7c6)cc5)cc4)cc3)ccc21 ; CC1(C)c2ccccc2-c2cc(-c3ccc(-c4ccc(N(c5ccc(-c6ccccc6)cc5)c5cccc(-c6cccc7oc8ccccc8c67)c5)cc4)cc3)ccc21]

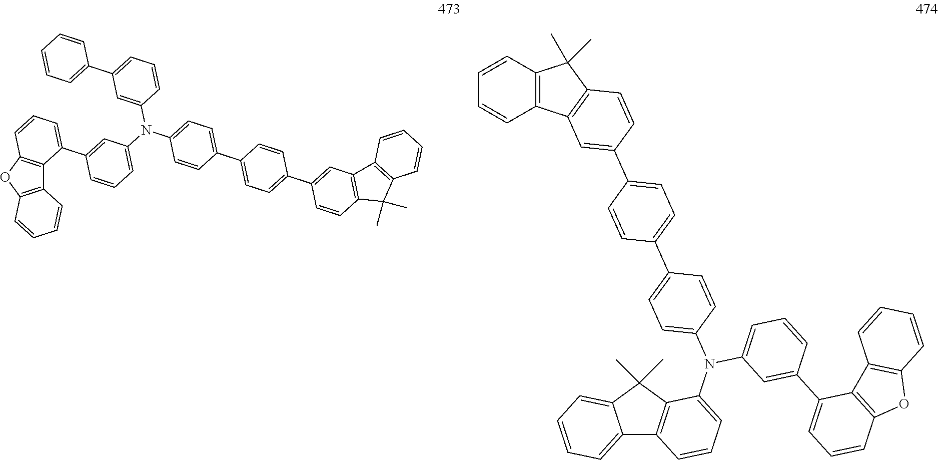 [SMILES: CC1(C)c2ccccc2-c2cc(-c3ccc(-c4ccc(N(c5cccc(-c6ccccc6)c5)c5cccc(-c6cccc7oc8ccccc8c67)c5)cc4)cc3)ccc21 ; CC1(C)c2ccccc2-c2cc(-c3ccc(-c4ccc(N(c5cccc(-c6cccc7oc8ccccc8c67)c5)c5cccc6c5C(C)(C)c5ccccc5-6)cc4)cc3)ccc21]

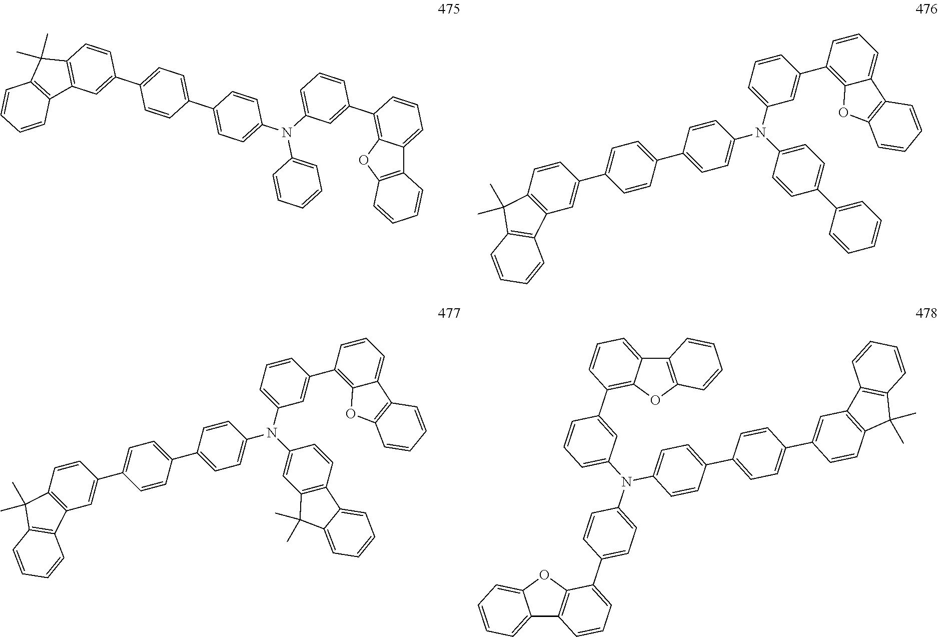 [SMILES: CC1(C)c2ccccc2-c2cc(-c3ccc(-c4ccc(N(c5ccccc5)c5cccc(-c6cccc7c6oc6ccccc67)c5)cc4)cc3)ccc21 ; CC1(C)c2ccccc2-c2cc(-c3ccc(-c4ccc(N(c5ccc(-c6ccccc6)cc5)c5cccc(-c6cccc7c6oc6ccccc67)c5)cc4)cc3)ccc21 ; CC1(C)c2ccccc2-c2cc(-c3ccc(-c4ccc(N(c5cccc(-c6cccc7c6oc6ccccc67)c5)c5ccc6c(c5)C(C)(C)c5ccccc5-6)cc4)cc3)ccc21 ; CC1(C)c2ccccc2-c2cc(-c3ccc(-c4ccc(N(c5ccc(-c6cccc7c6oc6ccccc67)cc5)c5cccc(-c6cccc7c6oc6ccccc67)c5)cc4)cc3)ccc21]

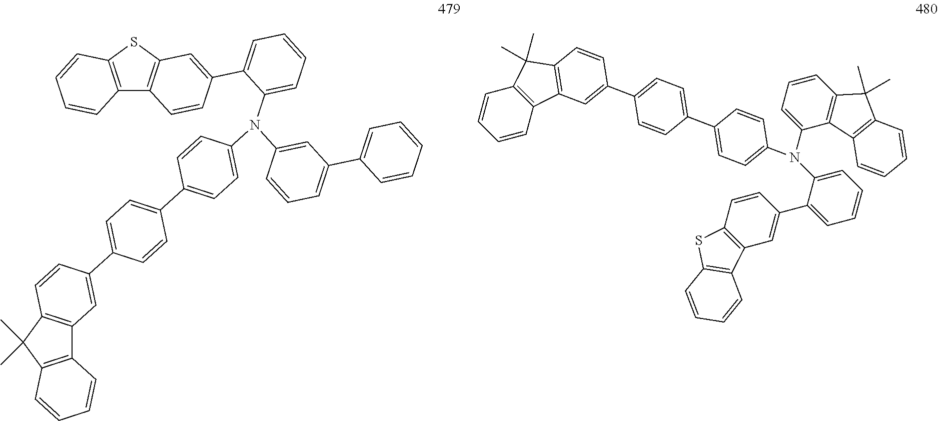 [SMILES: CC1(C)c2ccccc2-c2cc(-c3ccc(-c4ccc(N(c5cccc(-c6ccccc6)c5)c5ccccc5-c5ccc6c(c5)sc5ccccc56)cc4)cc3)ccc21 ; CC1(C)c2ccccc2-c2cc(-c3ccc(-c4ccc(N(c5ccccc5-c5ccc6sc7ccccc7c6c5)c5cccc6c5-c5ccccc5C6(C)C)cc4)cc3)ccc21]

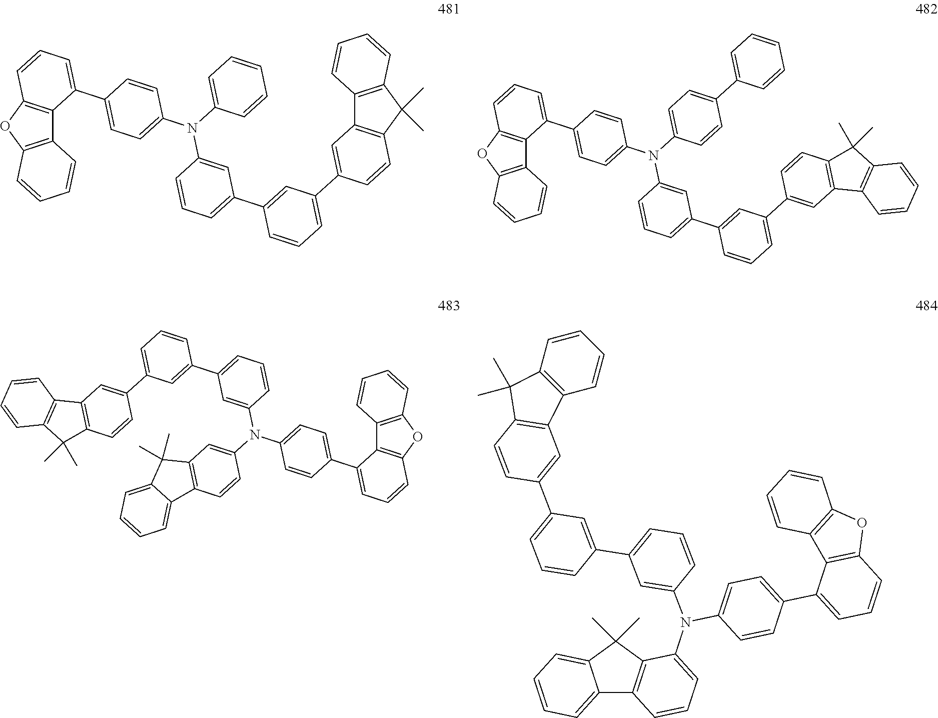 [SMILES: CC1(C)c2ccccc2-c2cc(-c3cccc(-c4cccc(N(c5ccccc5)c5ccc(-c6cccc7oc8ccccc8c67)cc5)c4)c3)ccc21 ; CC1(C)c2ccccc2-c2cc(-c3cccc(-c4cccc(N(c5ccc(-c6ccccc6)cc5)c5ccc(-c6cccc7oc8ccccc8c67)cc5)c4)c3)ccc21 ; CC1(C)c2ccccc2-c2cc(-c3cccc(-c4cccc(N(c5ccc(-c6cccc7oc8ccccc8c67)cc5)c5ccc6c(c5)C(C)(C)c5ccccc5-6)c4)c3)ccc21 ; CC1(C)c2ccccc2-c2cc(-c3cccc(-c4cccc(N(c5ccc(-c6cccc7oc8ccccc8c67)cc5)c5cccc6c5C(C)(C)c5ccccc5-6)c4)c3)ccc21]

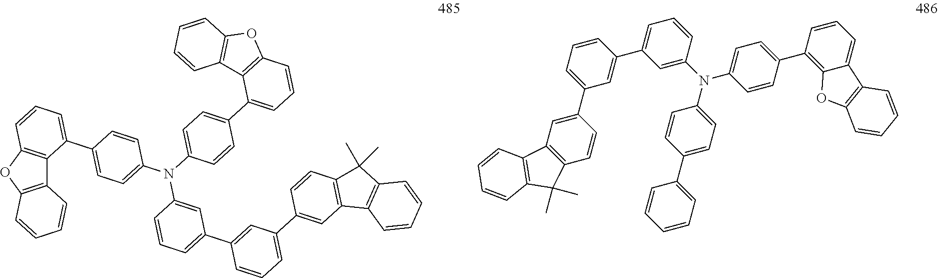 [SMILES: CC1(C)c2ccccc2-c2cc(-c3cccc(-c4cccc(N(c5ccc(-c6cccc7oc8ccccc8c67)cc5)c5ccc(-c6cccc7oc8ccccc8c67)cc5)c4)c3)ccc21 ; CC1(C)c2ccccc2-c2cc(-c3cccc(-c4cccc(N(c5ccc(-c6ccccc6)cc5)c5ccc(-c6cccc7c6oc6ccccc67)cc5)c4)c3)ccc21]

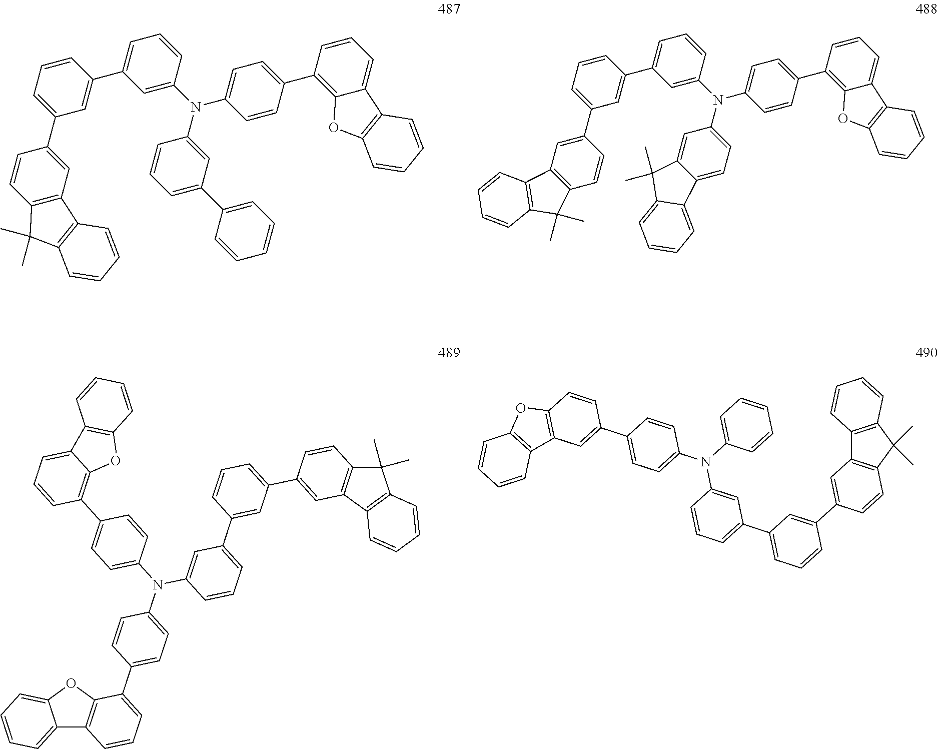 [SMILES: CC1(C)c2ccccc2-c2cc(-c3cccc(-c4cccc(N(c5ccc(-c6cccc7c6oc6ccccc67)cc5)c5cccc(-c6ccccc6)c5)c4)c3)ccc21 ; CC1(C)c2ccccc2-c2cc(-c3cccc(-c4cccc(N(c5ccc(-c6cccc7c6oc6ccccc67)cc5)c5ccc6c(c5)C(C)(C)c5ccccc5-6)c4)c3)ccc21 ; CC1(C)c2ccccc2-c2cc(-c3cccc(-c4cccc(N(c5ccc(-c6cccc7c6oc6ccccc67)cc5)c5ccc(-c6cccc7c6oc6ccccc67)cc5)c4)c3)ccc21 ; CC1(C)c2ccccc2-c2cc(-c3cccc(-c4cccc(N(c5ccccc5)c5ccc(-c6ccc7oc8ccccc8c7c6)cc5)c4)c3)ccc21]

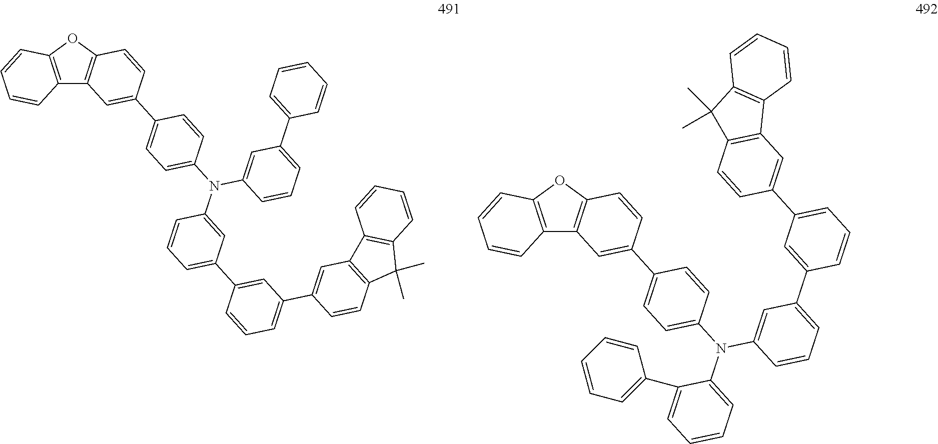 [SMILES: CC1(C)c2ccccc2-c2cc(-c3cccc(-c4cccc(N(c5ccc(-c6ccc7oc8ccccc8c7c6)cc5)c5cccc(-c6ccccc6)c5)c4)c3)ccc21 ; CC1(C)c2ccccc2-c2cc(-c3cccc(-c4cccc(N(c5ccc(-c6ccc7oc8ccccc8c7c6)cc5)c5ccccc5-c5ccccc5)c4)c3)ccc21]

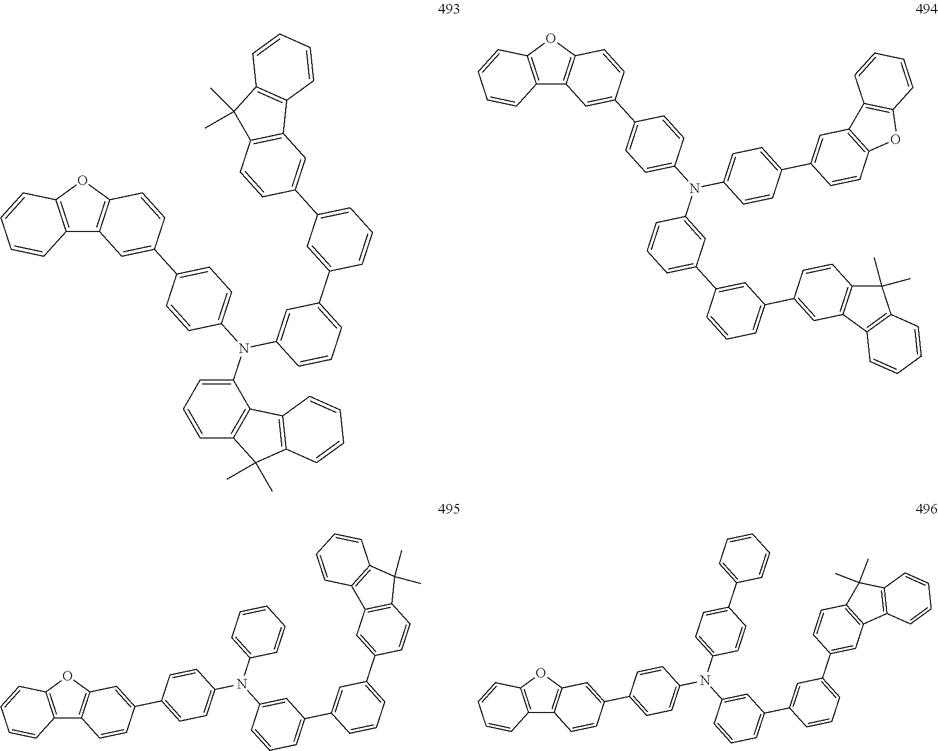 [SMILES: CC1(C)c2ccccc2-c2cc(-c3cccc(-c4cccc(N(c5ccc(-c6ccc7oc8ccccc8c7c6)cc5)c5cccc6c5-c5ccccc5C6(C)C)c4)c3)ccc21 ; CC1(C)c2ccccc2-c2cc(-c3cccc(-c4cccc(N(c5ccc(-c6ccc7oc8ccccc8c7c6)cc5)c5ccc(-c6ccc7oc8ccccc8c7c6)cc5)c4)c3)ccc21 ; CC1(C)c2ccccc2-c2cc(-c3cccc(-c4cccc(N(c5ccccc5)c5ccc(-c6ccc7c(c6)oc6ccccc67)cc5)c4)c3)ccc21 ; CC1(C)c2ccccc2-c2cc(-c3cccc(-c4cccc(N(c5ccc(-c6ccccc6)cc5)c5ccc(-c6ccc7c(c6)oc6ccccc67)cc5)c4)c3)ccc21]

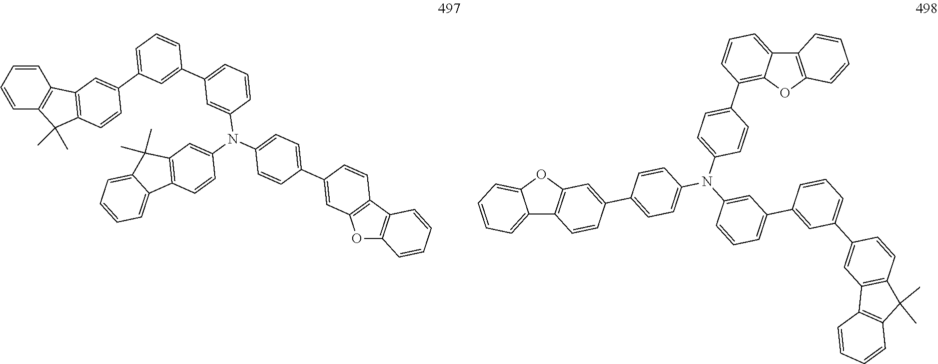 [SMILES: CC1(C)c2ccccc2-c2cc(-c3cccc(-c4cccc(N(c5ccc(-c6ccc7c(c6)oc6ccccc67)cc5)c5ccc6c(c5)C(C)(C)c5ccccc5-6)c4)c3)ccc21 ; CC1(C)c2ccccc2-c2cc(-c3cccc(-c4cccc(N(c5ccc(-c6ccc7c(c6)oc6ccccc67)cc5)c5ccc(-c6cccc7c6oc6ccccc67)cc5)c4)c3)ccc21]

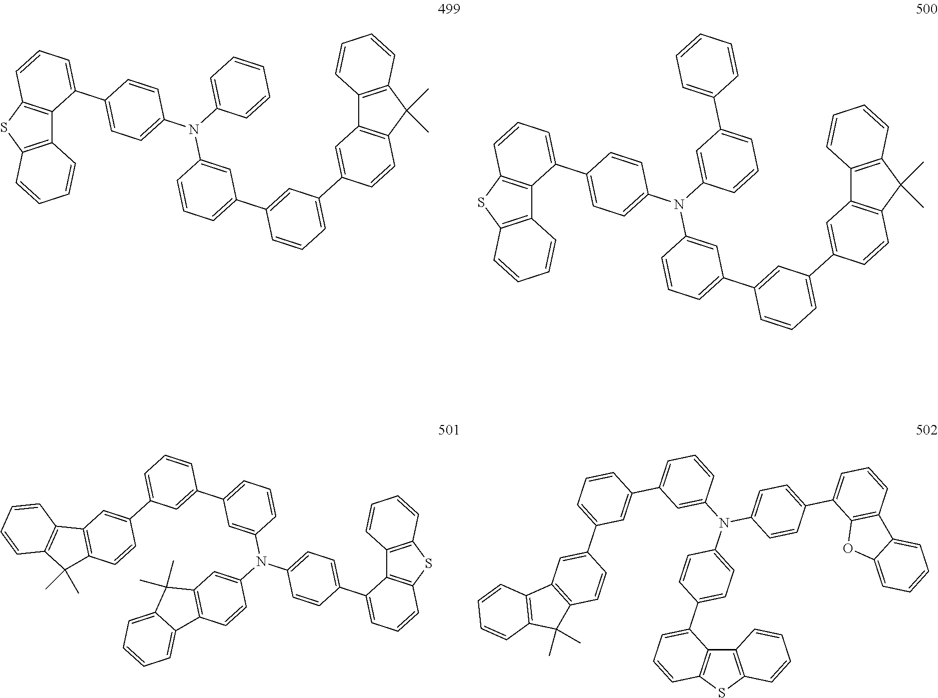 [SMILES: CC1(C)c2ccccc2-c2cc(-c3cccc(-c4cccc(N(c5ccccc5)c5ccc(-c6cccc7sc8ccccc8c67)cc5)c4)c3)ccc21 ; CC1(C)c2ccccc2-c2cc(-c3cccc(-c4cccc(N(c5ccc(-c6cccc7sc8ccccc8c67)cc5)c5cccc(-c6ccccc6)c5)c4)c3)ccc21 ; CC1(C)c2ccccc2-c2cc(-c3cccc(-c4cccc(N(c5ccc(-c6cccc7sc8ccccc8c67)cc5)c5ccc6c(c5)C(C)(C)c5ccccc5-6)c4)c3)ccc21 ; CC1(C)c2ccccc2-c2cc(-c3cccc(-c4cccc(N(c5ccc(-c6cccc7c6oc6ccccc67)cc5)c5ccc(-c6cccc7sc8ccccc8c67)cc5)c4)c3)ccc21]

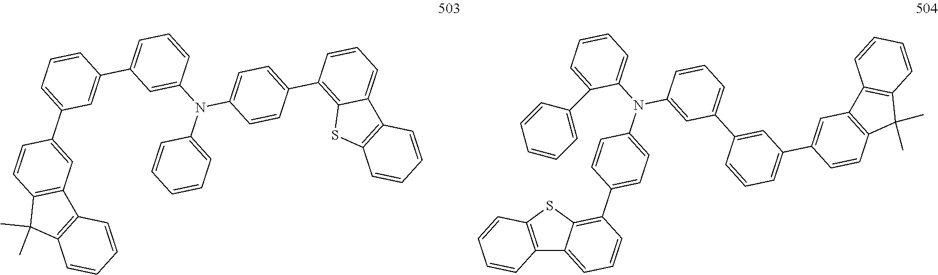 [SMILES: CC1(C)c2ccccc2-c2cc(-c3cccc(-c4cccc(N(c5ccccc5)c5ccc(-c6cccc7c6sc6ccccc67)cc5)c4)c3)ccc21 ; CC1(C)c2ccccc2-c2cc(-c3cccc(-c4cccc(N(c5ccc(-c6cccc7c6sc6ccccc67)cc5)c5ccccc5-c5ccccc5)c4)c3)ccc21]

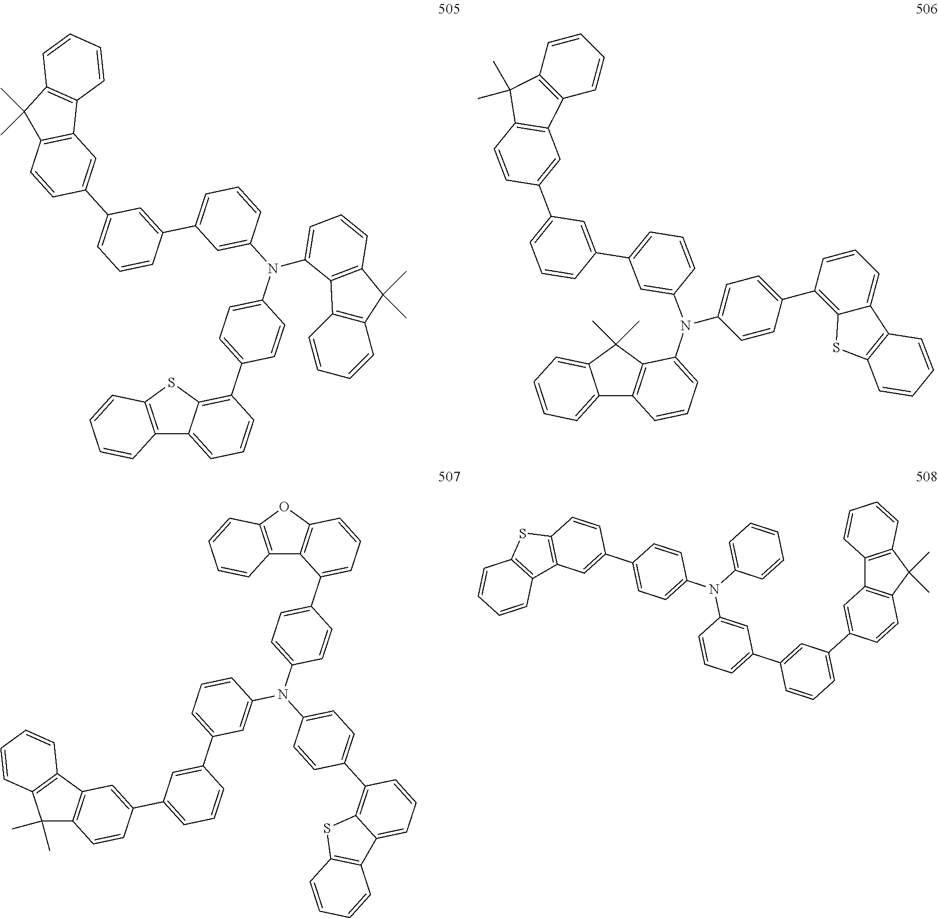

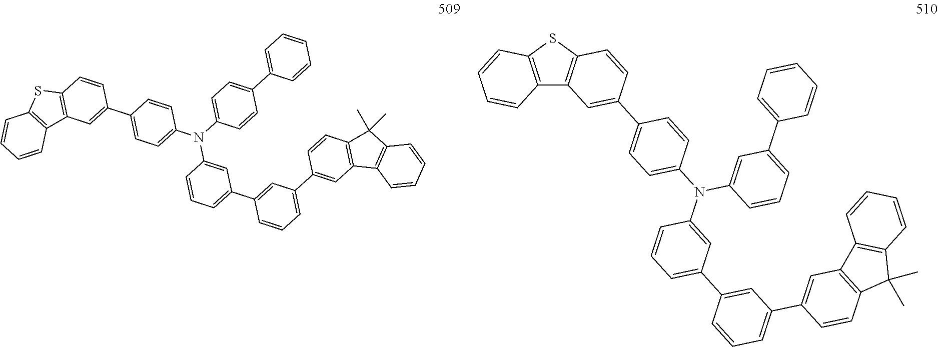 [SMILES: CC1(C)c2ccccc2-c2cc(-c3cccc(-c4cccc(N(c5ccc(-c6ccccc6)cc5)c5ccc(-c6ccc7sc8ccccc8c7c6)cc5)c4)c3)ccc21 ; CC1(C)c2ccccc2-c2cc(-c3cccc(-c4cccc(N(c5ccc(-c6ccc7sc8ccccc8c7c6)cc5)c5cccc(-c6ccccc6)c5)c4)c3)ccc21]

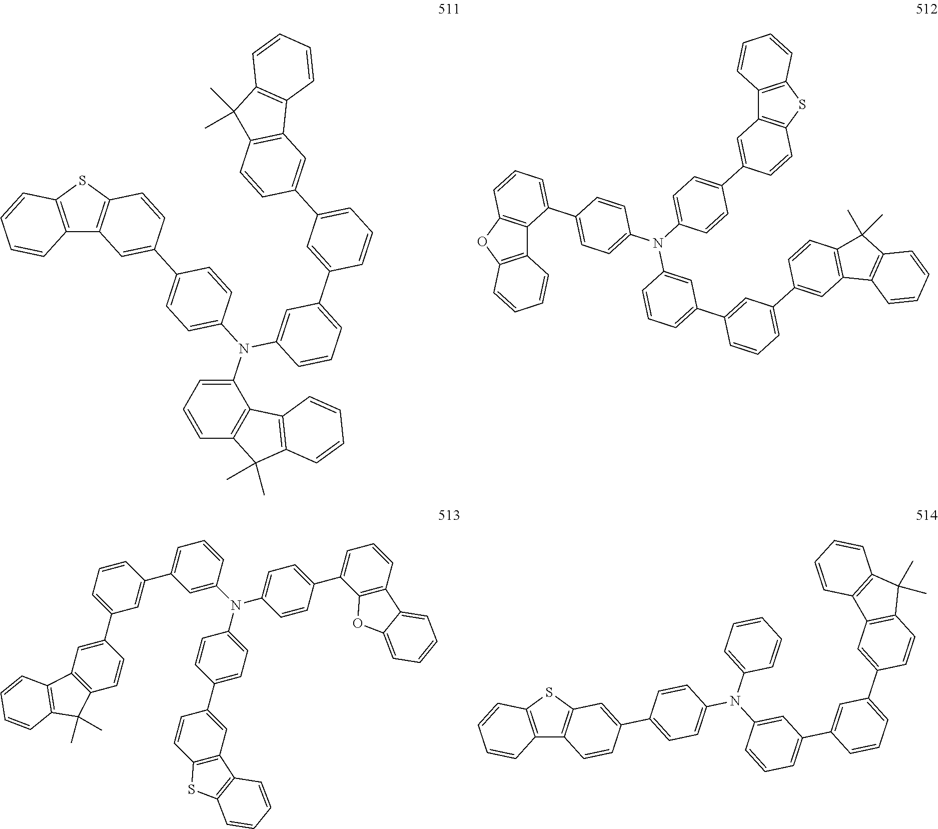 [SMILES: CC1(C)c2ccccc2-c2cc(-c3cccc(-c4cccc(N(c5ccc(-c6ccc7sc8ccccc8c7c6)cc5)c5cccc6c5-c5ccccc5C6(C)C)c4)c3)ccc21 ; CC1(C)c2ccccc2-c2cc(-c3cccc(-c4cccc(N(c5ccc(-c6ccc7sc8ccccc8c7c6)cc5)c5ccc(-c6cccc7oc8ccccc8c67)cc5)c4)c3)ccc21 ; CC1(C)c2ccccc2-c2cc(-c3cccc(-c4cccc(N(c5ccc(-c6ccc7sc8ccccc8c7c6)cc5)c5ccc(-c6cccc7c6oc6ccccc67)cc5)c4)c3)ccc21 ; CC1(C)c2ccccc2-c2cc(-c3cccc(-c4cccc(N(c5ccccc5)c5ccc(-c6ccc7c(c6)sc6ccccc67)cc5)c4)c3)ccc21]

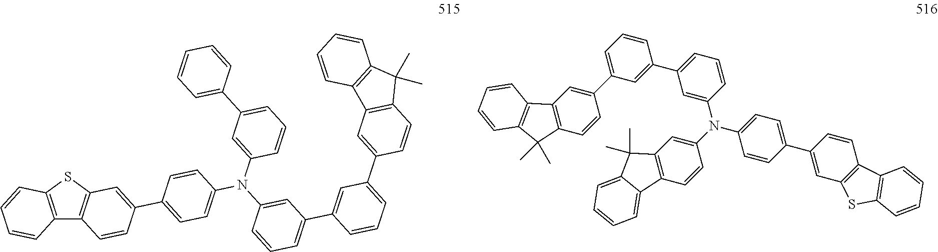 [SMILES: CC1(C)c2ccccc2-c2cc(-c3cccc(-c4cccc(N(c5ccc(-c6ccc7c(c6)sc6ccccc67)cc5)c5cccc(-c6ccccc6)c5)c4)c3)ccc21 ; CC1(C)c2ccccc2-c2cc(-c3cccc(-c4cccc(N(c5ccc(-c6ccc7c(c6)sc6ccccc67)cc5)c5ccc6c(c5)C(C)(C)c5ccccc5-6)c4)c3)ccc21]

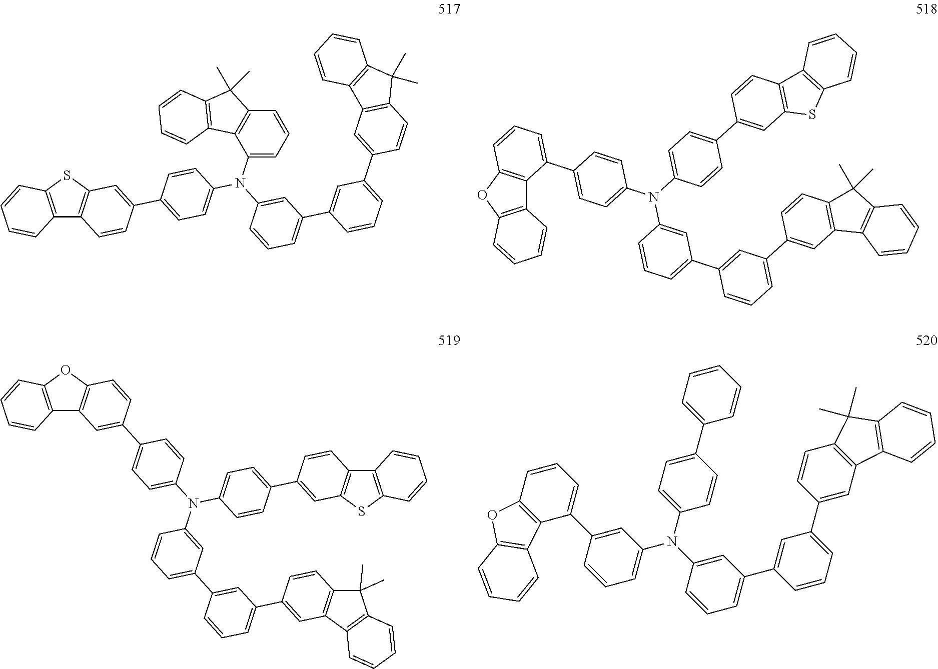 [SMILES: CC1(C)c2ccccc2-c2cc(-c3cccc(-c4cccc(N(c5ccc(-c6ccc7c(c6)sc6ccccc67)cc5)c5cccc6c5-c5ccccc5C6(C)C)c4)c3)ccc21 ; CC1(C)c2ccccc2-c2cc(-c3cccc(-c4cccc(N(c5ccc(-c6ccc7c(c6)sc6ccccc67)cc5)c5ccc(-c6cccc7oc8ccccc8c67)cc5)c4)c3)ccc21 ; CC1(C)c2ccccc2-c2cc(-c3cccc(-c4cccc(N(c5ccc(-c6ccc7c(c6)sc6ccccc67)cc5)c5ccc(-c6ccc7oc8ccccc8c7c6)cc5)c4)c3)ccc21 ; CC1(C)c2ccccc2-c2cc(-c3cccc(-c4cccc(N(c5ccc(-c6ccccc6)cc5)c5cccc(-c6cccc7oc8ccccc8c67)c5)c4)c3)ccc21]

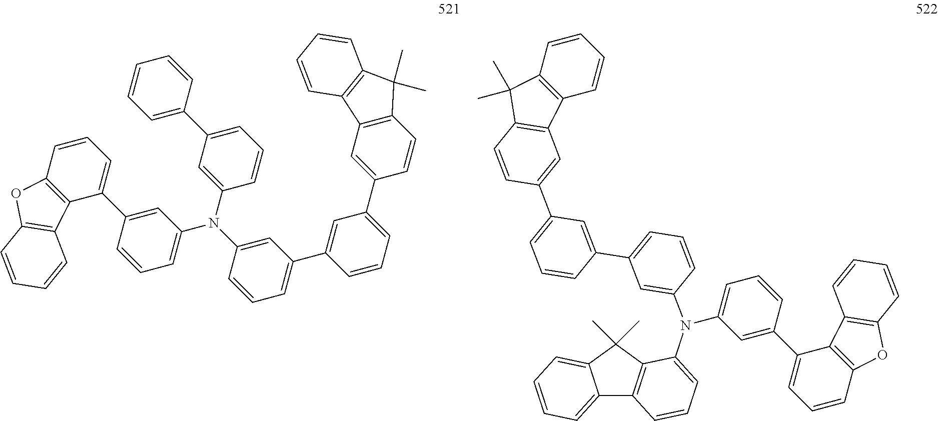 [SMILES: CC1(C)c2ccccc2-c2cc(-c3cccc(-c4cccc(N(c5cccc(-c6ccccc6)c5)c5cccc(-c6cccc7oc8ccccc8c67)c5)c4)c3)ccc21 ; CC1(C)c2ccccc2-c2cc(-c3cccc(-c4cccc(N(c5cccc(-c6cccc7oc8ccccc8c67)c5)c5cccc6c5C(C)(C)c5ccccc5-6)c4)c3)ccc21]

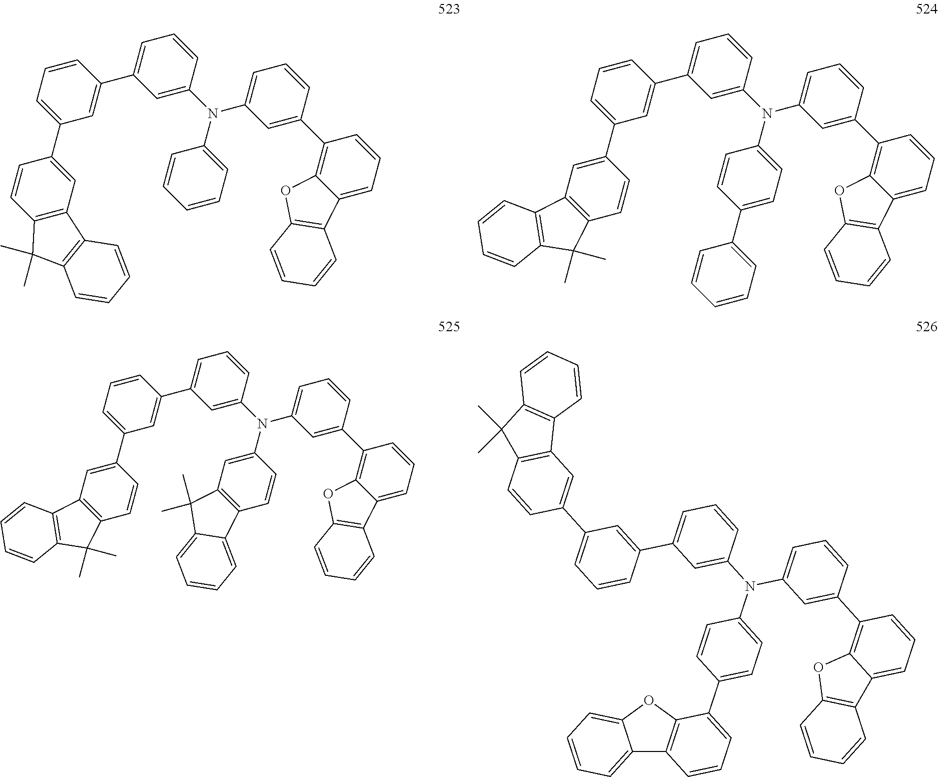 [SMILES: CC1(C)c2ccccc2-c2cc(-c3cccc(-c4cccc(N(c5ccccc5)c5cccc(-c6cccc7c6oc6ccccc67)c5)c4)c3)ccc21 ; CC1(C)c2ccccc2-c2cc(-c3cccc(-c4cccc(N(c5ccc(-c6ccccc6)cc5)c5cccc(-c6cccc7c6oc6ccccc67)c5)c4)c3)ccc21 ; CC1(C)c2ccccc2-c2cc(-c3cccc(-c4cccc(N(c5cccc(-c6cccc7c6oc6ccccc67)c5)c5ccc6c(c5)C(C)(C)c5ccccc5-6)c4)c3)ccc21 ; CC1(C)c2ccccc2-c2cc(-c3cccc(-c4cccc(N(c5ccc(-c6cccc7c6oc6ccccc67)cc5)c5cccc(-c6cccc7c6oc6ccccc67)c5)c4)c3)ccc21]

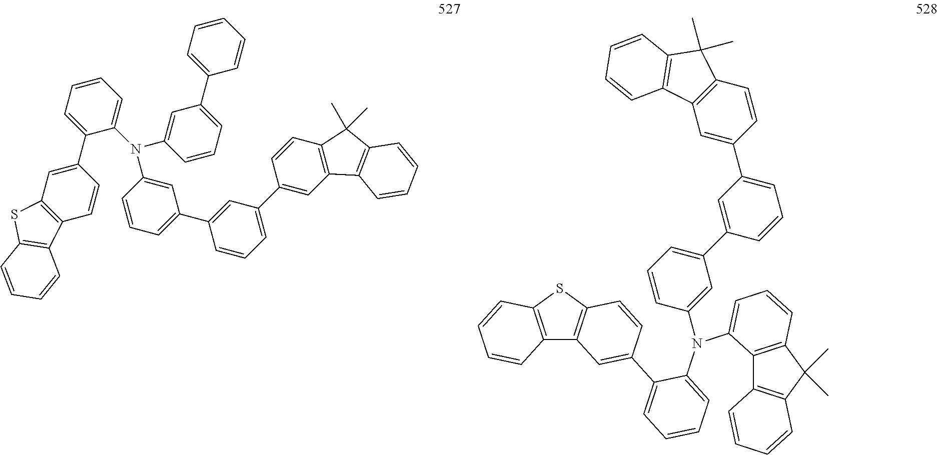 [SMILES: CC1(C)c2ccccc2-c2cc(-c3cccc(-c4cccc(N(c5cccc(-c6ccccc6)c5)c5ccccc5-c5ccc6c(c5)sc5ccccc56)c4)c3)ccc21 ; CC1(C)c2ccccc2-c2cc(-c3cccc(-c4cccc(N(c5ccccc5-c5ccc6sc7ccccc7c6c5)c5cccc6c5-c5ccccc5C6(C)C)c4)c3)ccc21]

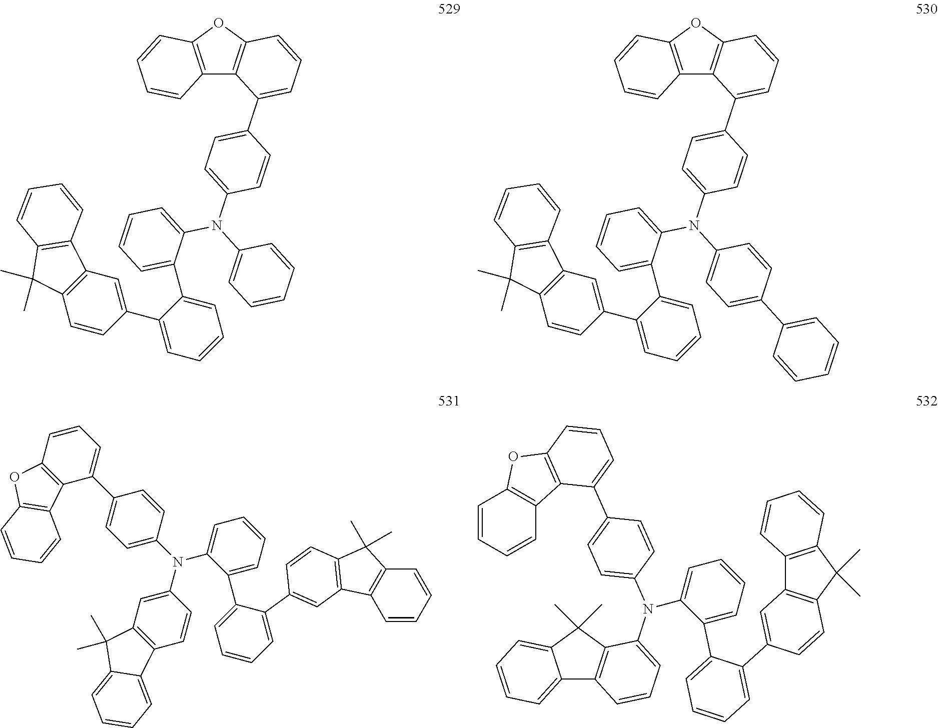 [SMILES: CC1(C)c2ccccc2-c2cc(-c3ccccc3-c3ccccc3N(c3ccccc3)c3ccc(-c4cccc5oc6ccccc6c45)cc3)ccc21 ; CC1(C)c2ccccc2-c2cc(-c3ccccc3-c3ccccc3N(c3ccc(-c4ccccc4)cc3)c3ccc(-c4cccc5oc6ccccc6c45)cc3)ccc21 ; CC1(C)c2ccccc2-c2cc(-c3ccccc3-c3ccccc3N(c3ccc(-c4cccc5oc6ccccc6c45)cc3)c3ccc4c(c3)C(C)(C)c3ccccc3-4)ccc21 ; CC1(C)c2ccccc2-c2cc(-c3ccccc3-c3ccccc3N(c3ccc(-c4cccc5oc6ccccc6c45)cc3)c3cccc4c3C(C)(C)c3ccccc3-4)ccc21]

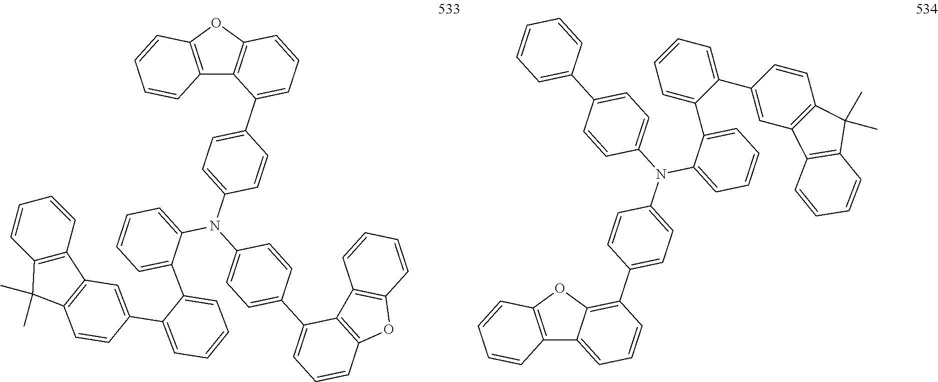 [SMILES: CC1(C)c2ccccc2-c2cc(-c3ccccc3-c3ccccc3N(c3ccc(-c4cccc5oc6ccccc6c45)cc3)c3ccc(-c4cccc5oc6ccccc6c45)cc3)ccc21 ; CC1(C)c2ccccc2-c2cc(-c3ccccc3-c3ccccc3N(c3ccc(-c4ccccc4)cc3)c3ccc(-c4cccc5c4oc4ccccc45)cc3)ccc21]

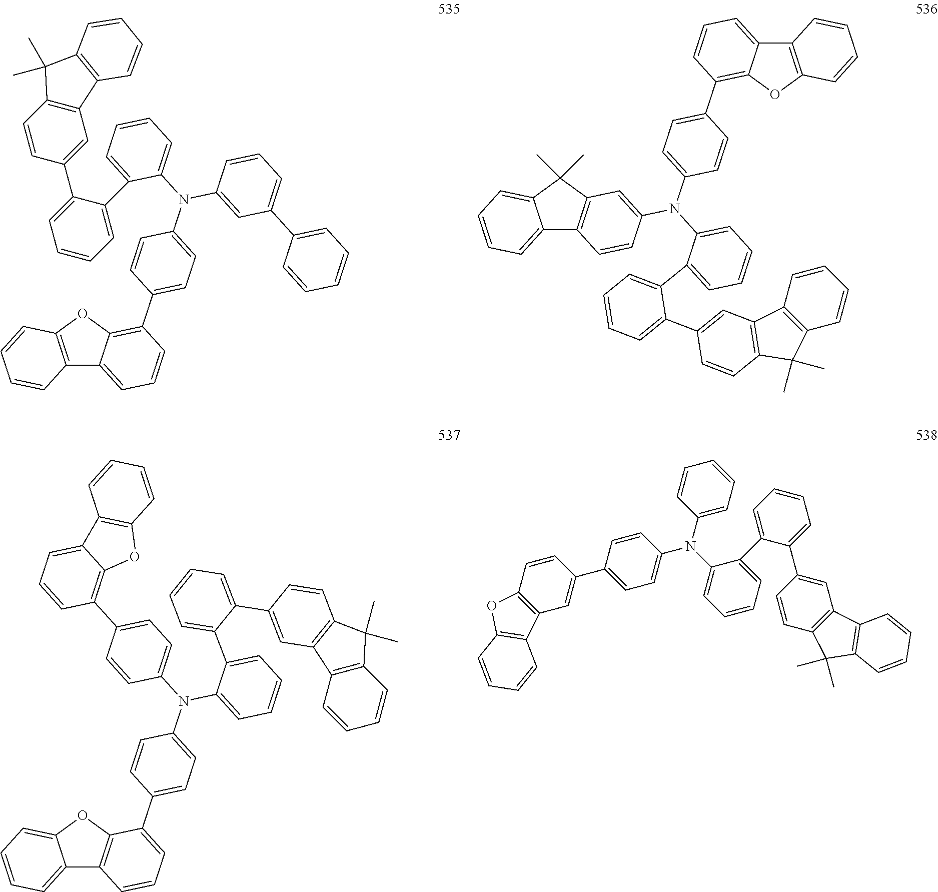 [SMILES: CC1(C)c2ccccc2-c2cc(-c3ccccc3-c3ccccc3N(c3ccc(-c4cccc5c4oc4ccccc45)cc3)c3cccc(-c4ccccc4)c3)ccc21 ; CC1(C)c2ccccc2-c2cc(-c3ccccc3-c3ccccc3N(c3ccc(-c4cccc5c4oc4ccccc45)cc3)c3ccc4c(c3)C(C)(C)c3ccccc3-4)ccc21 ; CC1(C)c2ccccc2-c2cc(-c3ccccc3-c3ccccc3N(c3ccc(-c4cccc5c4oc4ccccc45)cc3)c3ccc(-c4cccc5c4oc4ccccc45)cc3)ccc21 ; CC1(C)c2ccccc2-c2cc(-c3ccccc3-c3ccccc3N(c3ccccc3)c3ccc(-c4ccc5oc6ccccc6c5c4)cc3)ccc21]

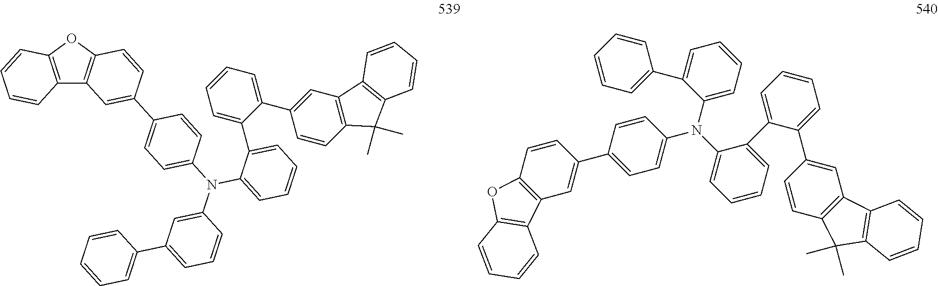 [SMILES: CC1(C)c2ccccc2-c2cc(-c3ccccc3-c3ccccc3N(c3ccc(-c4ccc5oc6ccccc6c5c4)cc3)c3cccc(-c4ccccc4)c3)ccc21 ; CC1(C)c2ccccc2-c2cc(-c3ccccc3-c3ccccc3N(c3ccc(-c4ccc5oc6ccccc6c5c4)cc3)c3ccccc3-c3ccccc3)ccc21]

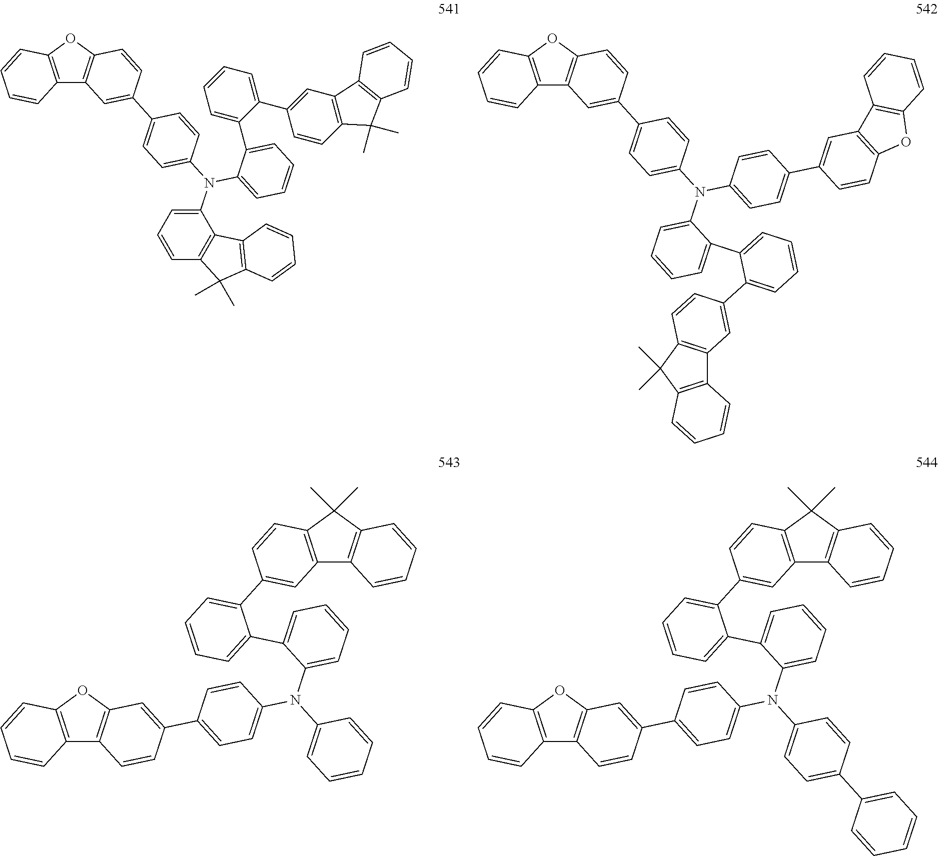 [SMILES: CC1(C)c2ccccc2-c2cc(-c3ccccc3-c3ccccc3N(c3ccc(-c4ccc5oc6ccccc6c5c4)cc3)c3cccc4c3-c3ccccc3C4(C)C)ccc21 ; CC1(C)c2ccccc2-c2cc(-c3ccccc3-c3ccccc3N(c3ccc(-c4ccc5oc6ccccc6c5c4)cc3)c3ccc(-c4ccc5oc6ccccc6c5c4)cc3)ccc21 ; CC1(C)c2ccccc2-c2cc(-c3ccccc3-c3ccccc3N(c3ccccc3)c3ccc(-c4ccc5c(c4)oc4ccccc45)cc3)ccc21 ; CC1(C)c2ccccc2-c2cc(-c3ccccc3-c3ccccc3N(c3ccc(-c4ccccc4)cc3)c3ccc(-c4ccc5c(c4)oc4ccccc45)cc3)ccc21]

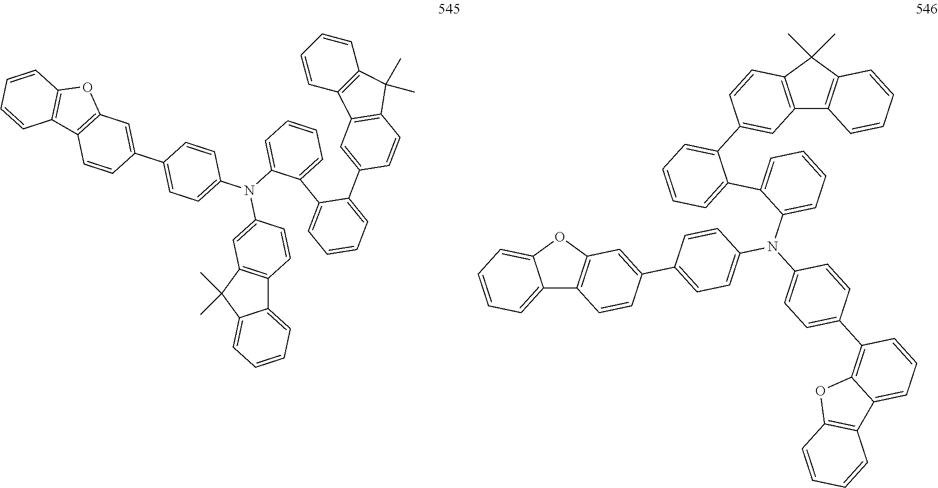 [SMILES: CC1(C)c2ccccc2-c2cc(-c3ccccc3-c3ccccc3N(c3ccc(-c4ccc5c(c4)oc4ccccc45)cc3)c3ccc4c(c3)C(C)(C)c3ccccc3-4)ccc21 ; CC1(C)c2ccccc2-c2cc(-c3ccccc3-c3ccccc3N(c3ccc(-c4ccc5c(c4)oc4ccccc45)cc3)c3ccc(-c4cccc5c4oc4ccccc45)cc3)ccc21]

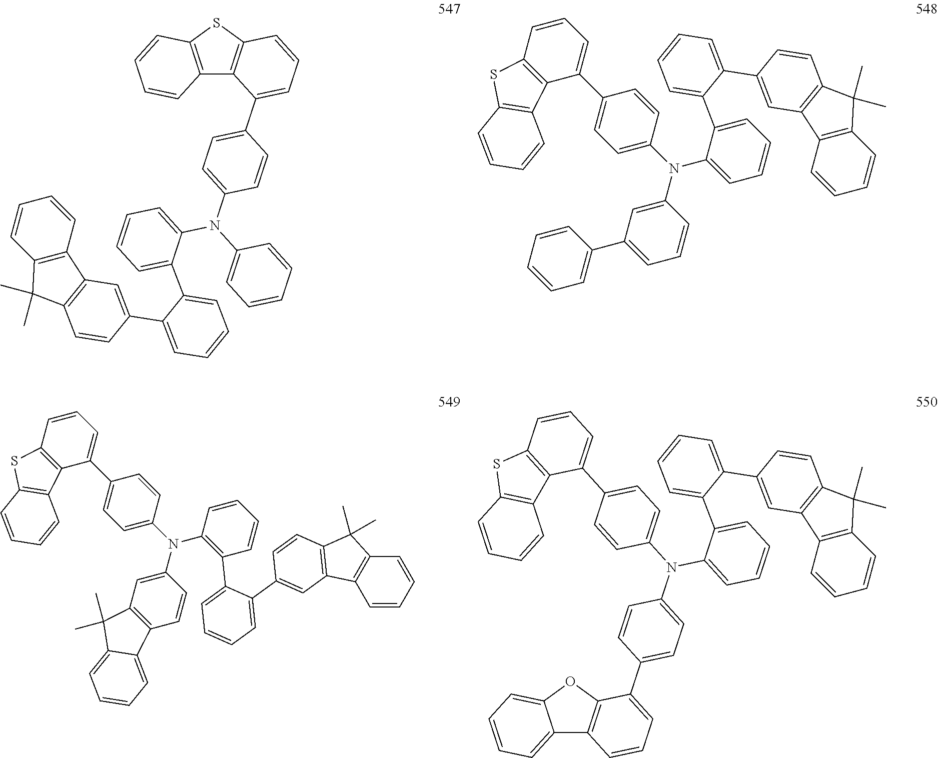 [SMILES: CC1(C)c2ccccc2-c2cc(-c3ccccc3-c3ccccc3N(c3ccccc3)c3ccc(-c4cccc5sc6ccccc6c45)cc3)ccc21 ; CC1(C)c2ccccc2-c2cc(-c3ccccc3-c3ccccc3N(c3ccc(-c4cccc5sc6ccccc6c45)cc3)c3cccc(-c4ccccc4)c3)ccc21 ; CC1(C)c2ccccc2-c2cc(-c3ccccc3-c3ccccc3N(c3ccc(-c4cccc5sc6ccccc6c45)cc3)c3ccc4c(c3)C(C)(C)c3ccccc3-4)ccc21 ; CC1(C)c2ccccc2-c2cc(-c3ccccc3-c3ccccc3N(c3ccc(-c4cccc5c4oc4ccccc45)cc3)c3ccc(-c4cccc5sc6ccccc6c45)cc3)ccc21]

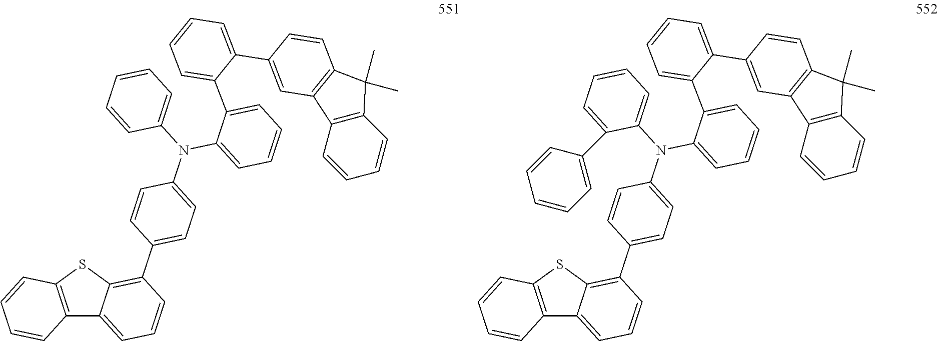 [SMILES: CC1(C)c2ccccc2-c2cc(-c3ccccc3-c3ccccc3N(c3ccccc3)c3ccc(-c4cccc5c4sc4ccccc45)cc3)ccc21 ; CC1(C)c2ccccc2-c2cc(-c3ccccc3-c3ccccc3N(c3ccc(-c4cccc5c4sc4ccccc45)cc3)c3ccccc3-c3ccccc3)ccc21]

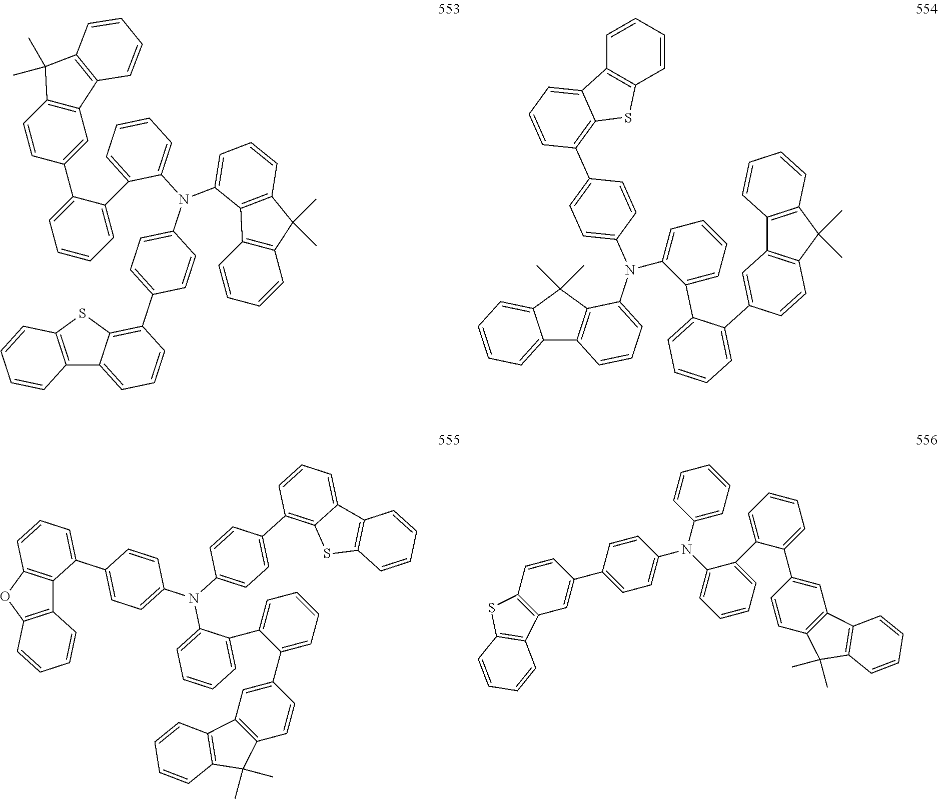 [SMILES: CC1(C)c2ccccc2-c2cc(-c3ccccc3-c3ccccc3N(c3ccc(-c4cccc5c4sc4ccccc45)cc3)c3cccc4c3-c3ccccc3C4(C)C)ccc21 ; CC1(C)c2ccccc2-c2cc(-c3ccccc3-c3ccccc3N(c3ccc(-c4cccc5c4sc4ccccc45)cc3)c3cccc4c3C(C)(C)c3ccccc3-4)ccc21 ; CC1(C)c2ccccc2-c2cc(-c3ccccc3-c3ccccc3N(c3ccc(-c4cccc5c4sc4ccccc45)cc3)c3ccc(-c4cccc5oc6ccccc6c45)cc3)ccc21 ; CC1(C)c2ccccc2-c2cc(-c3ccccc3-c3ccccc3N(c3ccccc3)c3ccc(-c4ccc5sc6ccccc6c5c4)cc3)ccc21]

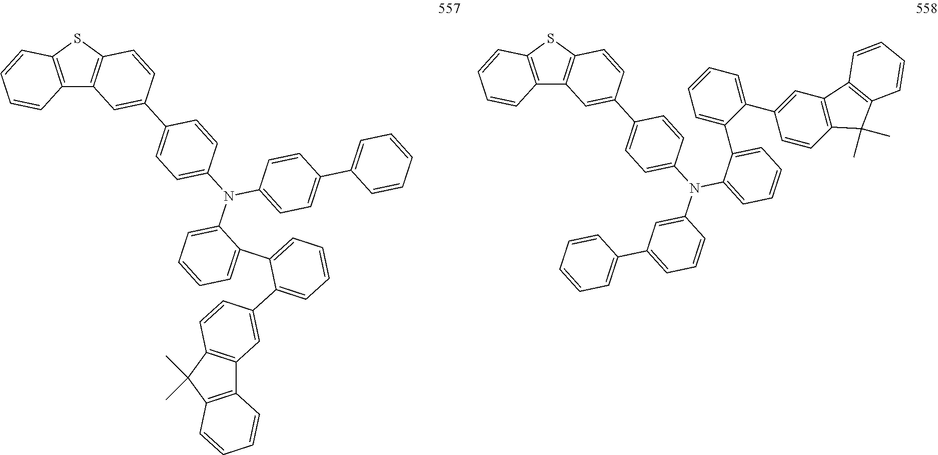 [SMILES: CC1(C)c2ccccc2-c2cc(-c3ccccc3-c3ccccc3N(c3ccc(-c4ccccc4)cc3)c3ccc(-c4ccc5sc6ccccc6c5c4)cc3)ccc21 ; CC1(C)c2ccccc2-c2cc(-c3ccccc3-c3ccccc3N(c3ccc(-c4ccc5sc6ccccc6c5c4)cc3)c3cccc(-c4ccccc4)c3)ccc21]

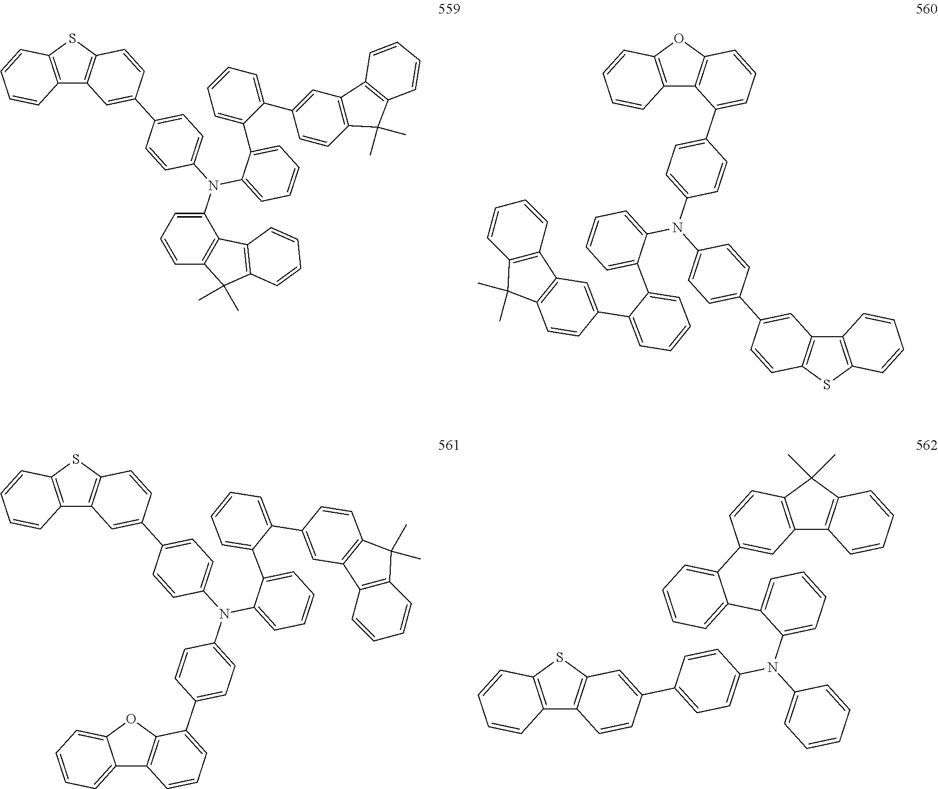 [SMILES: CC1(C)c2ccccc2-c2cc(-c3ccccc3-c3ccccc3N(c3ccc(-c4ccc5sc6ccccc6c5c4)cc3)c3cccc4c3-c3ccccc3C4(C)C)ccc21 ; CC1(C)c2ccccc2-c2cc(-c3ccccc3-c3ccccc3N(c3ccc(-c4ccc5sc6ccccc6c5c4)cc3)c3ccc(-c4cccc5oc6ccccc6c45)cc3)ccc21 ; CC1(C)c2ccccc2-c2cc(-c3ccccc3-c3ccccc3N(c3ccc(-c4ccc5sc6ccccc6c5c4)cc3)c3ccc(-c4cccc5c4oc4ccccc45)cc3)ccc21 ; CC1(C)c2ccccc2-c2cc(-c3ccccc3-c3ccccc3N(c3ccccc3)c3ccc(-c4ccc5c(c4)sc4ccccc45)cc3)ccc21]

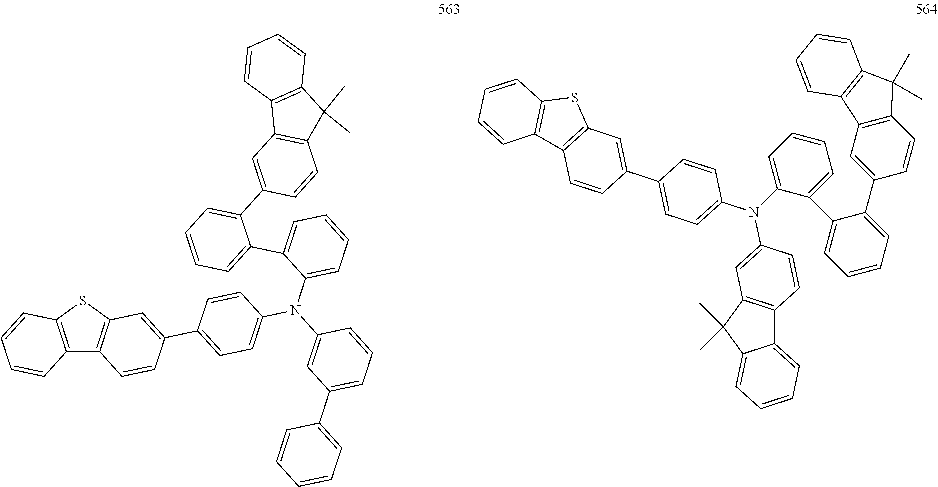 [SMILES: CC1(C)c2ccccc2-c2cc(-c3ccccc3-c3ccccc3N(c3ccc(-c4ccc5c(c4)sc4ccccc45)cc3)c3cccc(-c4ccccc4)c3)ccc21 ; CC1(C)c2ccccc2-c2cc(-c3ccccc3-c3ccccc3N(c3ccc(-c4ccc5c(c4)sc4ccccc45)cc3)c3ccc4c(c3)C(C)(C)c3ccccc3-4)ccc21]

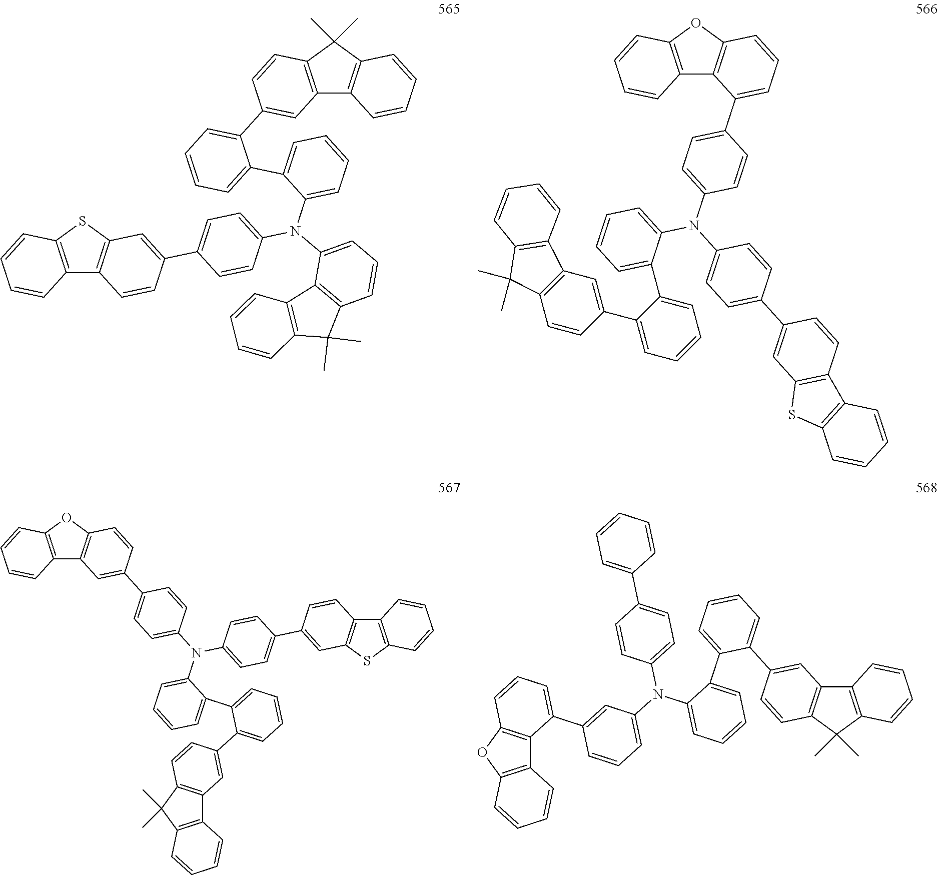 [SMILES: CC1(C)c2ccccc2-c2cc(-c3ccccc3-c3ccccc3N(c3ccc(-c4ccc5c(c4)sc4ccccc45)cc3)c3cccc4c3-c3ccccc3C4(C)C)ccc21 ; CC1(C)c2ccccc2-c2cc(-c3ccccc3-c3ccccc3N(c3ccc(-c4ccc5c(c4)sc4ccccc45)cc3)c3ccc(-c4cccc5oc6ccccc6c45)cc3)ccc21 ; CC1(C)c2ccccc2-c2cc(-c3ccccc3-c3ccccc3N(c3ccc(-c4ccc5c(c4)sc4ccccc45)cc3)c3ccc(-c4ccc5oc6ccccc6c5c4)cc3)ccc21 ; CC1(C)c2ccccc2-c2cc(-c3ccccc3-c3ccccc3N(c3ccc(-c4ccccc4)cc3)c3cccc(-c4cccc5oc6ccccc6c45)c3)ccc21]

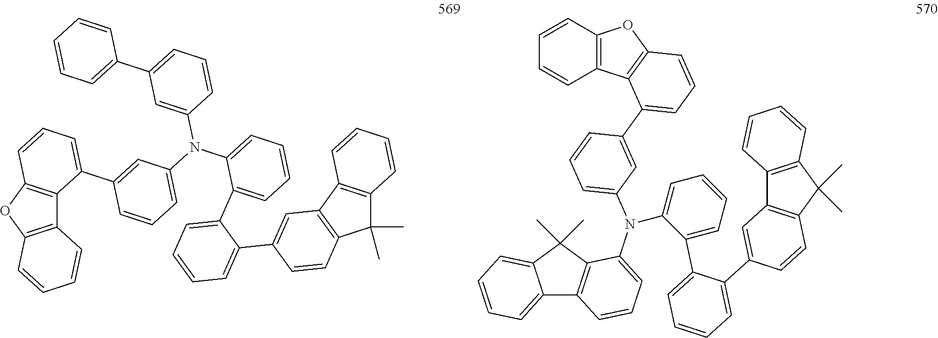 [SMILES: CC1(C)c2ccccc2-c2cc(-c3ccccc3-c3ccccc3N(c3cccc(-c4ccccc4)c3)c3cccc(-c4cccc5oc6ccccc6c45)c3)ccc21 ; CC1(C)c2ccccc2-c2cc(-c3ccccc3-c3ccccc3N(c3cccc(-c4cccc5oc6ccccc6c45)c3)c3cccc4c3C(C)(C)c3ccccc3-4)ccc21]

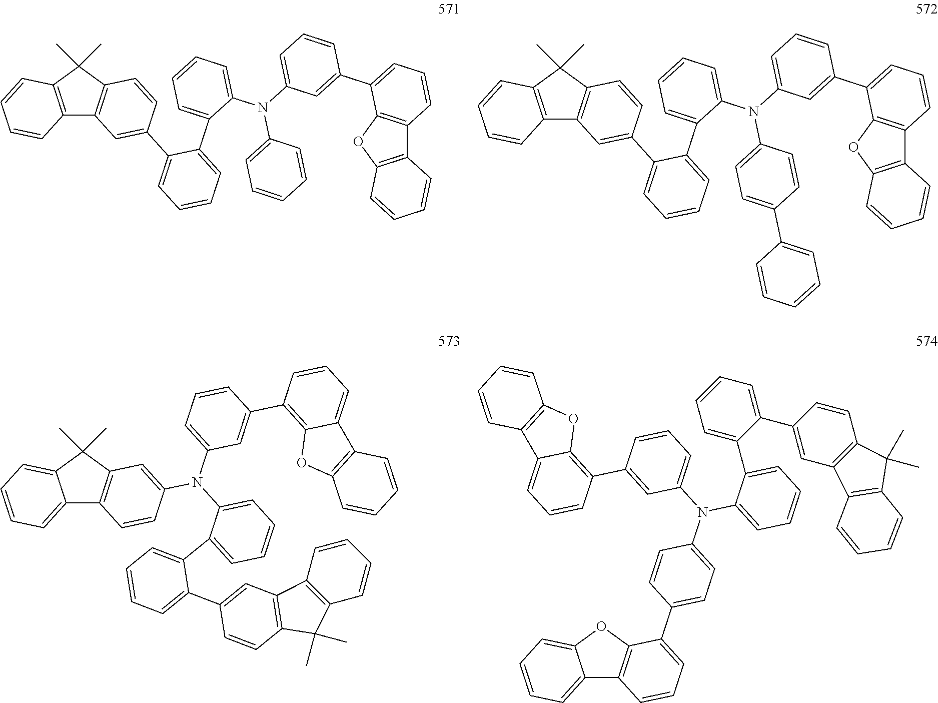 [SMILES: CC1(C)c2ccccc2-c2cc(-c3ccccc3-c3ccccc3N(c3ccccc3)c3cccc(-c4cccc5c4oc4ccccc45)c3)ccc21 ; CC1(C)c2ccccc2-c2cc(-c3ccccc3-c3ccccc3N(c3ccc(-c4ccccc4)cc3)c3cccc(-c4cccc5c4oc4ccccc45)c3)ccc21 ; CC1(C)c2ccccc2-c2cc(-c3ccccc3-c3ccccc3N(c3cccc(-c4cccc5c4oc4ccccc45)c3)c3ccc4c(c3)C(C)(C)c3ccccc3-4)ccc21 ; CC1(C)c2ccccc2-c2cc(-c3ccccc3-c3ccccc3N(c3ccc(-c4cccc5c4oc4ccccc45)cc3)c3cccc(-c4cccc5c4oc4ccccc45)c3)ccc21]

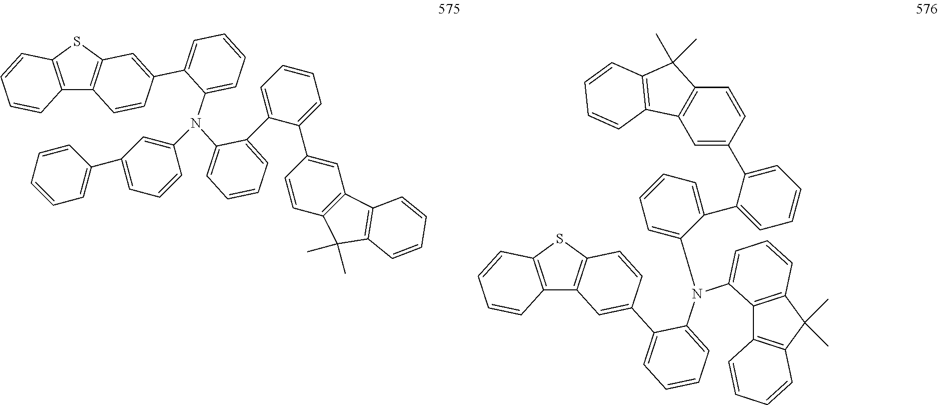 [SMILES: CC1(C)c2ccccc2-c2cc(-c3ccccc3-c3ccccc3N(c3cccc(-c4ccccc4)c3)c3ccccc3-c3ccc4c(c3)sc3ccccc34)ccc21 ; CC1(C)c2ccccc2-c2cc(-c3ccccc3-c3ccccc3N(c3ccccc3-c3ccc4sc5ccccc5c4c3)c3cccc4c3-c3ccccc3C4(C)C)ccc21]

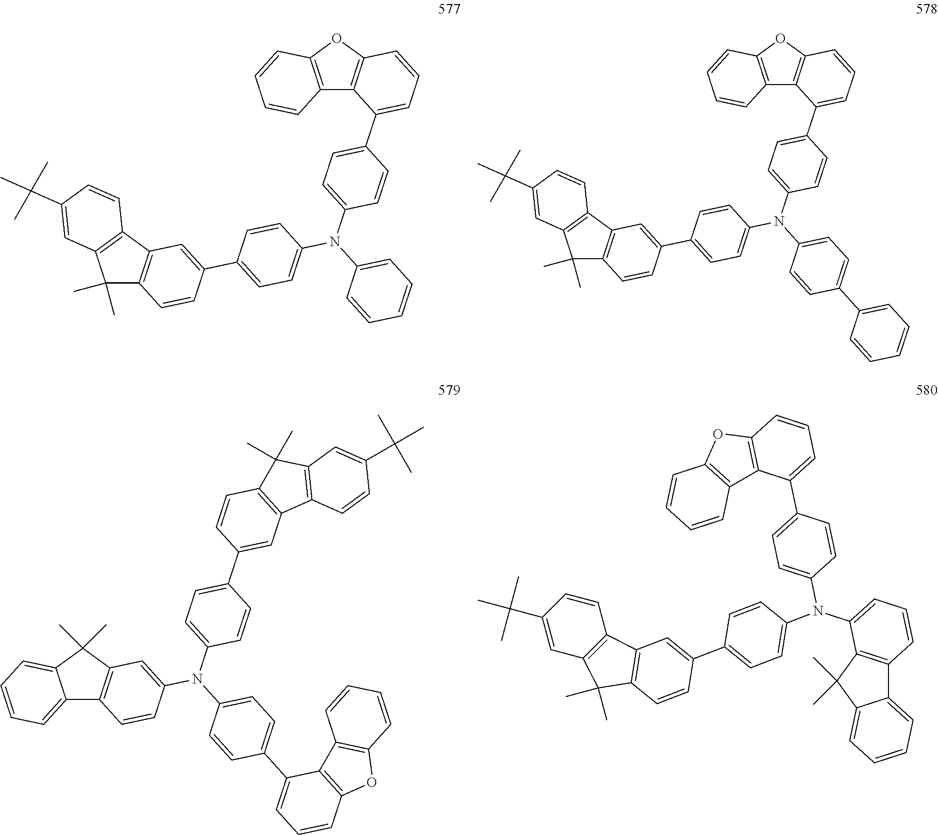 [SMILES: CC(C)(C)c1ccc2c(c1)C(C)(C)c1ccc(-c3ccc(N(c4ccccc4)c4ccc(-c5cccc6oc7ccccc7c56)cc4)cc3)cc1-2 ; CC(C)(C)c1ccc2c(c1)C(C)(C)c1ccc(-c3ccc(N(c4ccc(-c5ccccc5)cc4)c4ccc(-c5cccc6oc7ccccc7c56)cc4)cc3)cc1-2 ; CC(C)(C)c1ccc2c(c1)C(C)(C)c1ccc(-c3ccc(N(c4ccc(-c5cccc6oc7ccccc7c56)cc4)c4ccc5c(c4)C(C)(C)c4ccccc4-5)cc3)cc1-2 ; CC(C)(C)c1ccc2c(c1)C(C)(C)c1ccc(-c3ccc(N(c4ccc(-c5cccc6oc7ccccc7c56)cc4)c4cccc5c4C(C)(C)c4ccccc4-5)cc3)cc1-2]

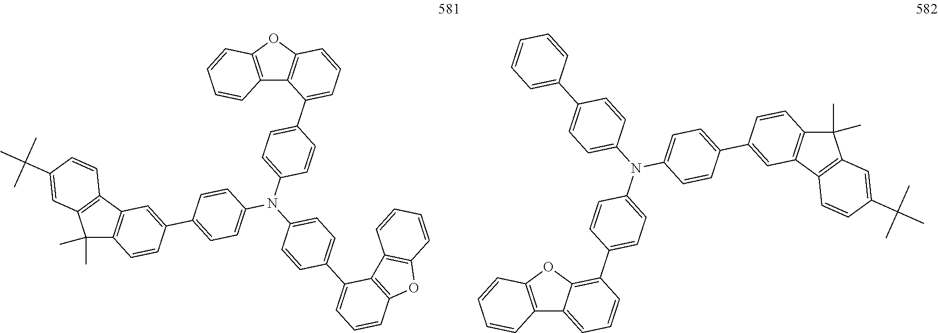 [SMILES: CC(C)(C)c1ccc2c(c1)C(C)(C)c1ccc(-c3ccc(N(c4ccc(-c5cccc6oc7ccccc7c56)cc4)c4ccc(-c5cccc6oc7ccccc7c56)cc4)cc3)cc1-2 ; CC(C)(C)c1ccc2c(c1)C(C)(C)c1ccc(-c3ccc(N(c4ccc(-c5ccccc5)cc4)c4ccc(-c5cccc6c5oc5ccccc56)cc4)cc3)cc1-2]

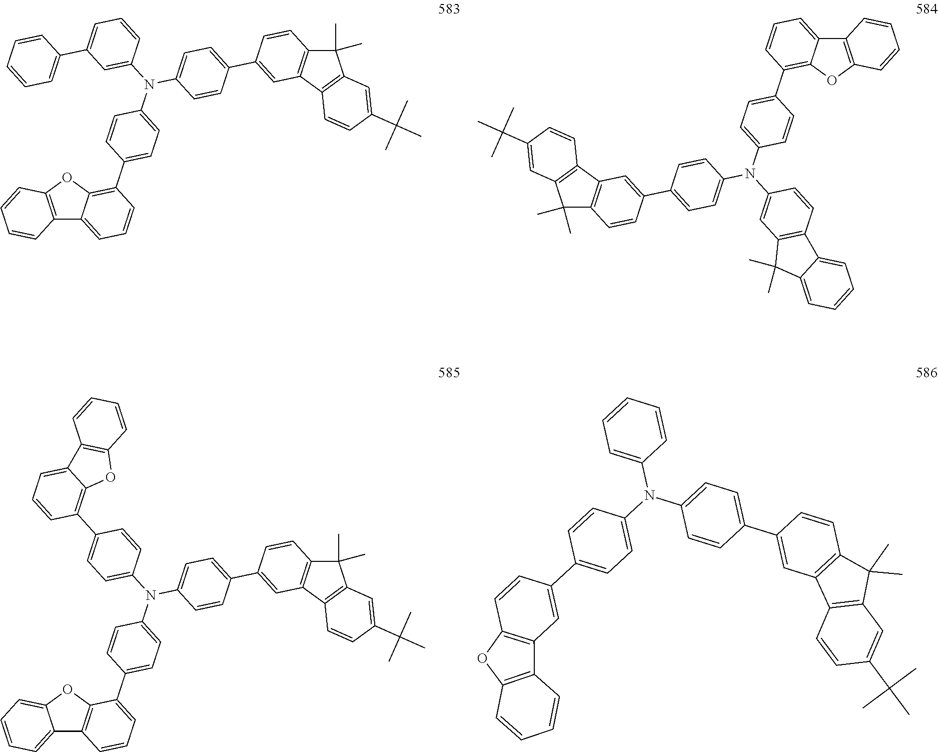 [SMILES: CC(C)(C)c1ccc2c(c1)C(C)(C)c1ccc(-c3ccc(N(c4ccc(-c5cccc6c5oc5ccccc56)cc4)c4cccc(-c5ccccc5)c4)cc3)cc1-2 ; CC(C)(C)c1ccc2c(c1)C(C)(C)c1ccc(-c3ccc(N(c4ccc(-c5cccc6c5oc5ccccc56)cc4)c4ccc5c(c4)C(C)(C)c4ccccc4-5)cc3)cc1-2 ; CC(C)(C)c1ccc2c(c1)C(C)(C)c1ccc(-c3ccc(N(c4ccc(-c5cccc6c5oc5ccccc56)cc4)c4ccc(-c5cccc6c5oc5ccccc56)cc4)cc3)cc1-2 ; CC(C)(C)c1ccc2c(c1)C(C)(C)c1ccc(-c3ccc(N(c4ccccc4)c4ccc(-c5ccc6oc7ccccc7c6c5)cc4)cc3)cc1-2]

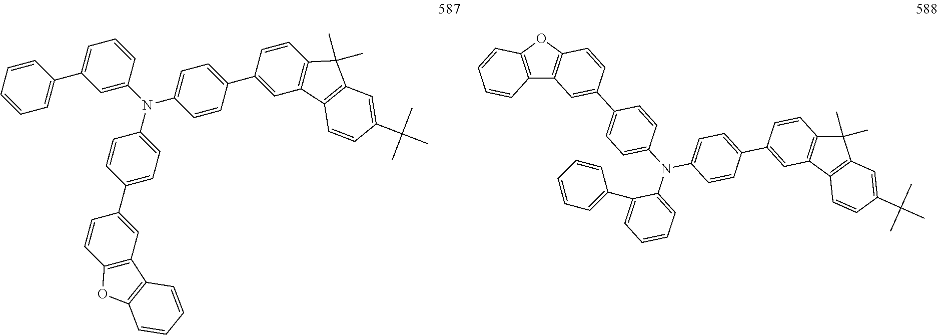 [SMILES: CC(C)(C)c1ccc2c(c1)C(C)(C)c1ccc(-c3ccc(N(c4ccc(-c5ccc6oc7ccccc7c6c5)cc4)c4cccc(-c5ccccc5)c4)cc3)cc1-2 ; CC(C)(C)c1ccc2c(c1)C(C)(C)c1ccc(-c3ccc(N(c4ccc(-c5ccc6oc7ccccc7c6c5)cc4)c4ccccc4-c4ccccc4)cc3)cc1-2]

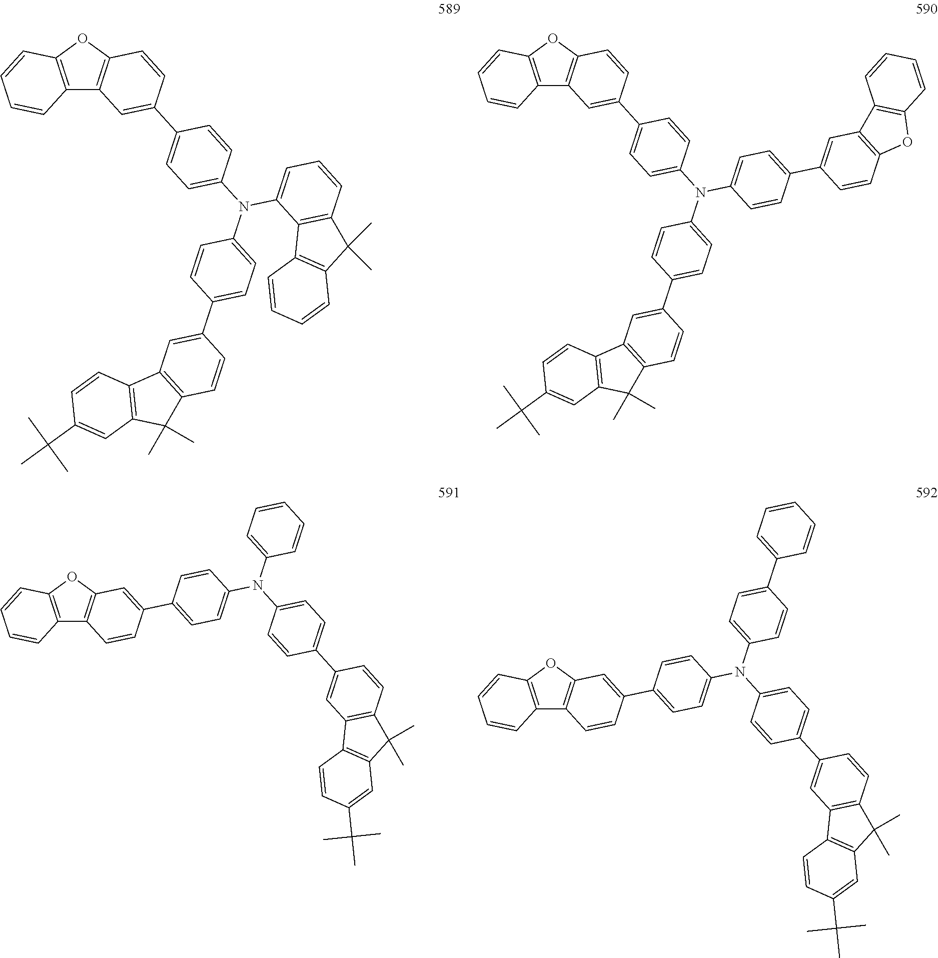 [SMILES: CC(C)(C)c1ccc2c(c1)C(C)(C)c1ccc(-c3ccc(N(c4ccc(-c5ccc6oc7ccccc7c6c5)cc4)c4cccc5c4-c4ccccc4C5(C)C)cc3)cc1-2 ; CC(C)(C)c1ccc2c(c1)C(C)(C)c1ccc(-c3ccc(N(c4ccc(-c5ccc6oc7ccccc7c6c5)cc4)c4ccc(-c5ccc6oc7ccccc7c6c5)cc4)cc3)cc1-2 ; CC(C)(C)c1ccc2c(c1)C(C)(C)c1ccc(-c3ccc(N(c4ccccc4)c4ccc(-c5ccc6c(c5)oc5ccccc56)cc4)cc3)cc1-2 ; CC(C)(C)c1ccc2c(c1)C(C)(C)c1ccc(-c3ccc(N(c4ccc(-c5ccccc5)cc4)c4ccc(-c5ccc6c(c5)oc5ccccc56)cc4)cc3)cc1-2]

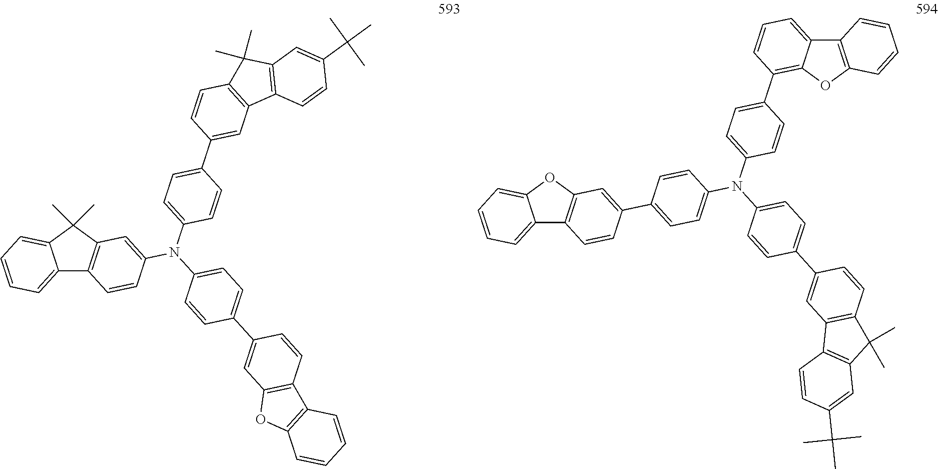 [SMILES: CC(C)(C)c1ccc2c(c1)C(C)(C)c1ccc(-c3ccc(N(c4ccc(-c5ccc6c(c5)oc5ccccc56)cc4)c4ccc5c(c4)C(C)(C)c4ccccc4-5)cc3)cc1-2 ; CC(C)(C)c1ccc2c(c1)C(C)(C)c1ccc(-c3ccc(N(c4ccc(-c5ccc6c(c5)oc5ccccc56)cc4)c4ccc(-c5cccc6c5oc5ccccc56)cc4)cc3)cc1-2]

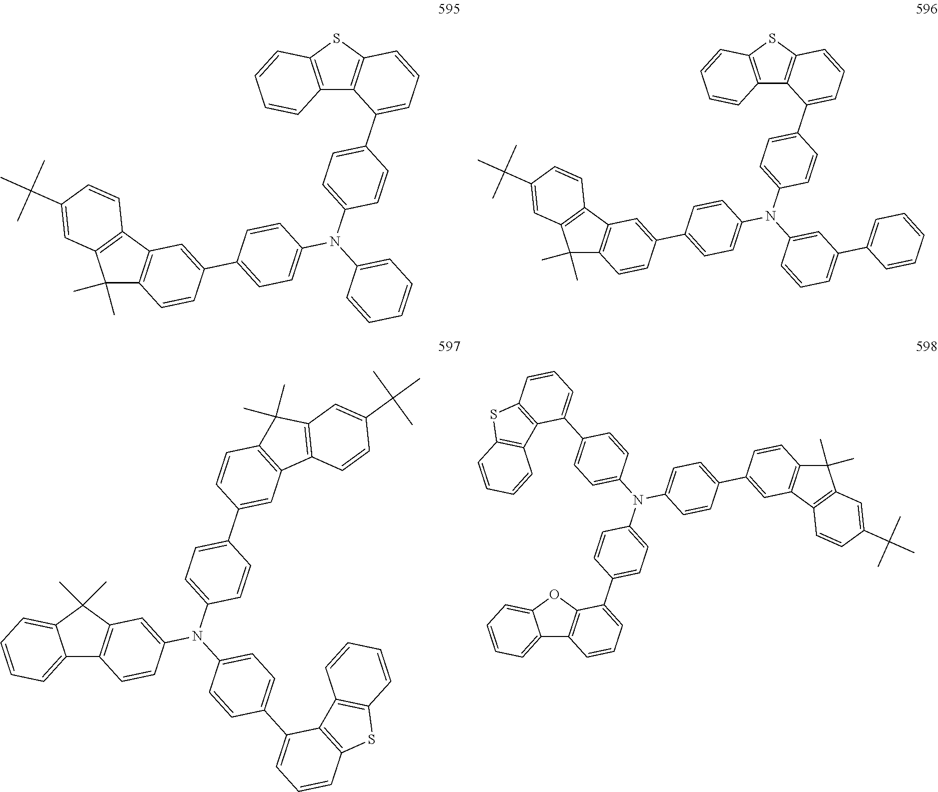 [SMILES: CC(C)(C)c1ccc2c(c1)C(C)(C)c1ccc(-c3ccc(N(c4ccccc4)c4ccc(-c5cccc6sc7ccccc7c56)cc4)cc3)cc1-2 ; CC(C)(C)c1ccc2c(c1)C(C)(C)c1ccc(-c3ccc(N(c4ccc(-c5cccc6sc7ccccc7c56)cc4)c4cccc(-c5ccccc5)c4)cc3)cc1-2 ; CC(C)(C)c1ccc2c(c1)C(C)(C)c1ccc(-c3ccc(N(c4ccc(-c5cccc6sc7ccccc7c56)cc4)c4ccc5c(c4)C(C)(C)c4ccccc4-5)cc3)cc1-2 ; CC(C)(C)c1ccc2c(c1)C(C)(C)c1ccc(-c3ccc(N(c4ccc(-c5cccc6c5oc5ccccc56)cc4)c4ccc(-c5cccc6sc7ccccc7c56)cc4)cc3)cc1-2]

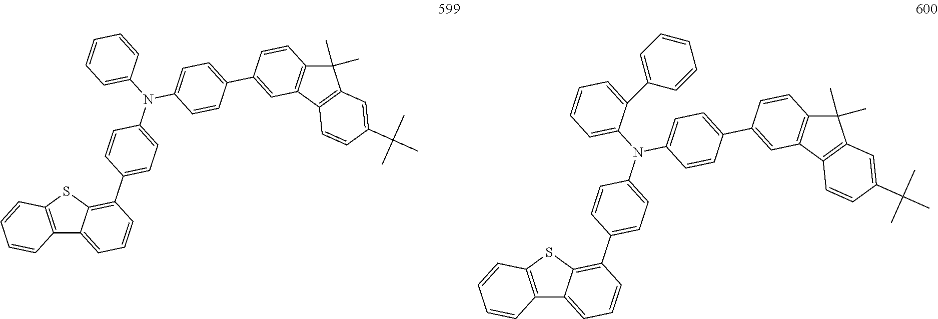 [SMILES: CC(C)(C)c1ccc2c(c1)C(C)(C)c1ccc(-c3ccc(N(c4ccccc4)c4ccc(-c5cccc6c5sc5ccccc56)cc4)cc3)cc1-2 ; CC(C)(C)c1ccc2c(c1)C(C)(C)c1ccc(-c3ccc(N(c4ccc(-c5cccc6c5sc5ccccc56)cc4)c4ccccc4-c4ccccc4)cc3)cc1-2]

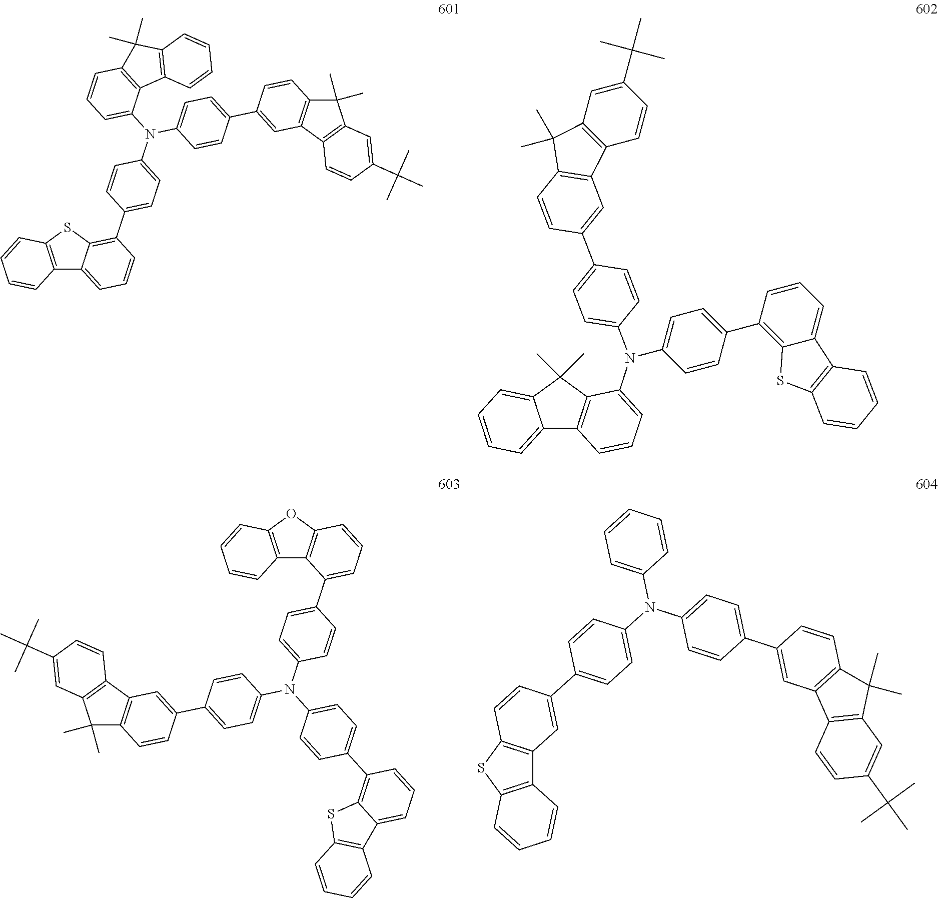 [SMILES: CC(C)(C)c1ccc2c(c1)C(C)(C)c1ccc(-c3ccc(N(c4ccc(-c5cccc6c5sc5ccccc56)cc4)c4cccc5c4-c4ccccc4C5(C)C)cc3)cc1-2 ; CC(C)(C)c1ccc2c(c1)C(C)(C)c1ccc(-c3ccc(N(c4ccc(-c5cccc6c5sc5ccccc56)cc4)c4cccc5c4C(C)(C)c4ccccc4-5)cc3)cc1-2 ; CC(C)(C)c1ccc2c(c1)C(C)(C)c1ccc(-c3ccc(N(c4ccc(-c5cccc6c5sc5ccccc56)cc4)c4ccc(-c5cccc6oc7ccccc7c56)cc4)cc3)cc1-2 ; CC(C)(C)c1ccc2c(c1)C(C)(C)c1ccc(-c3ccc(N(c4ccccc4)c4ccc(-c5ccc6sc7ccccc7c6c5)cc4)cc3)cc1-2]

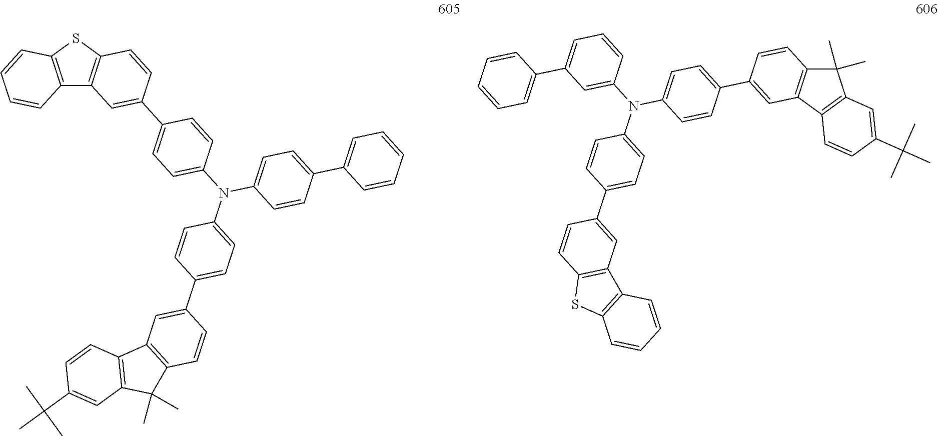 [SMILES: CC(C)(C)c1ccc2c(c1)C(C)(C)c1ccc(-c3ccc(N(c4ccc(-c5ccccc5)cc4)c4ccc(-c5ccc6sc7ccccc7c6c5)cc4)cc3)cc1-2 ; CC(C)(C)c1ccc2c(c1)C(C)(C)c1ccc(-c3ccc(N(c4ccc(-c5ccc6sc7ccccc7c6c5)cc4)c4cccc(-c5ccccc5)c4)cc3)cc1-2]

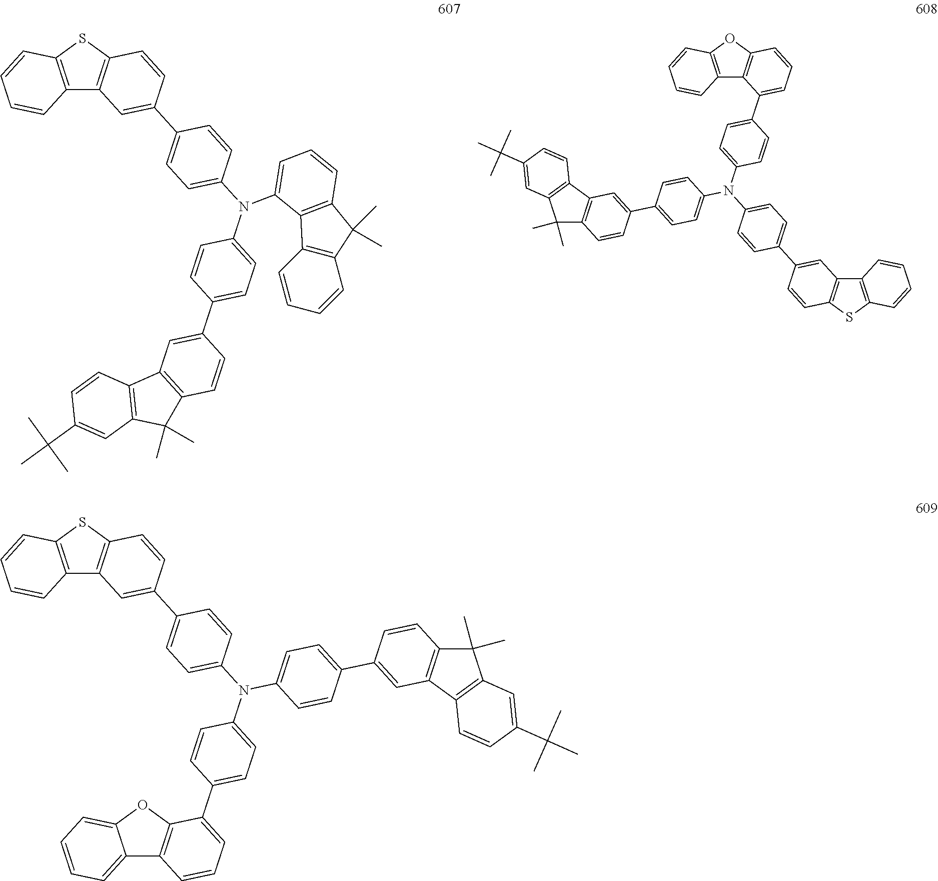 [SMILES: CC(C)(C)c1ccc2c(c1)C(C)(C)c1ccc(-c3ccc(N(c4ccc(-c5ccc6sc7ccccc7c6c5)cc4)c4cccc5c4-c4ccccc4C5(C)C)cc3)cc1-2 ; CC(C)(C)c1ccc2c(c1)C(C)(C)c1ccc(-c3ccc(N(c4ccc(-c5ccc6sc7ccccc7c6c5)cc4)c4ccc(-c5cccc6oc7ccccc7c56)cc4)cc3)cc1-2 ; CC(C)(C)c1ccc2c(c1)C(C)(C)c1ccc(-c3ccc(N(c4ccc(-c5ccc6sc7ccccc7c6c5)cc4)c4ccc(-c5cccc6c5oc5ccccc56)cc4)cc3)cc1-2]

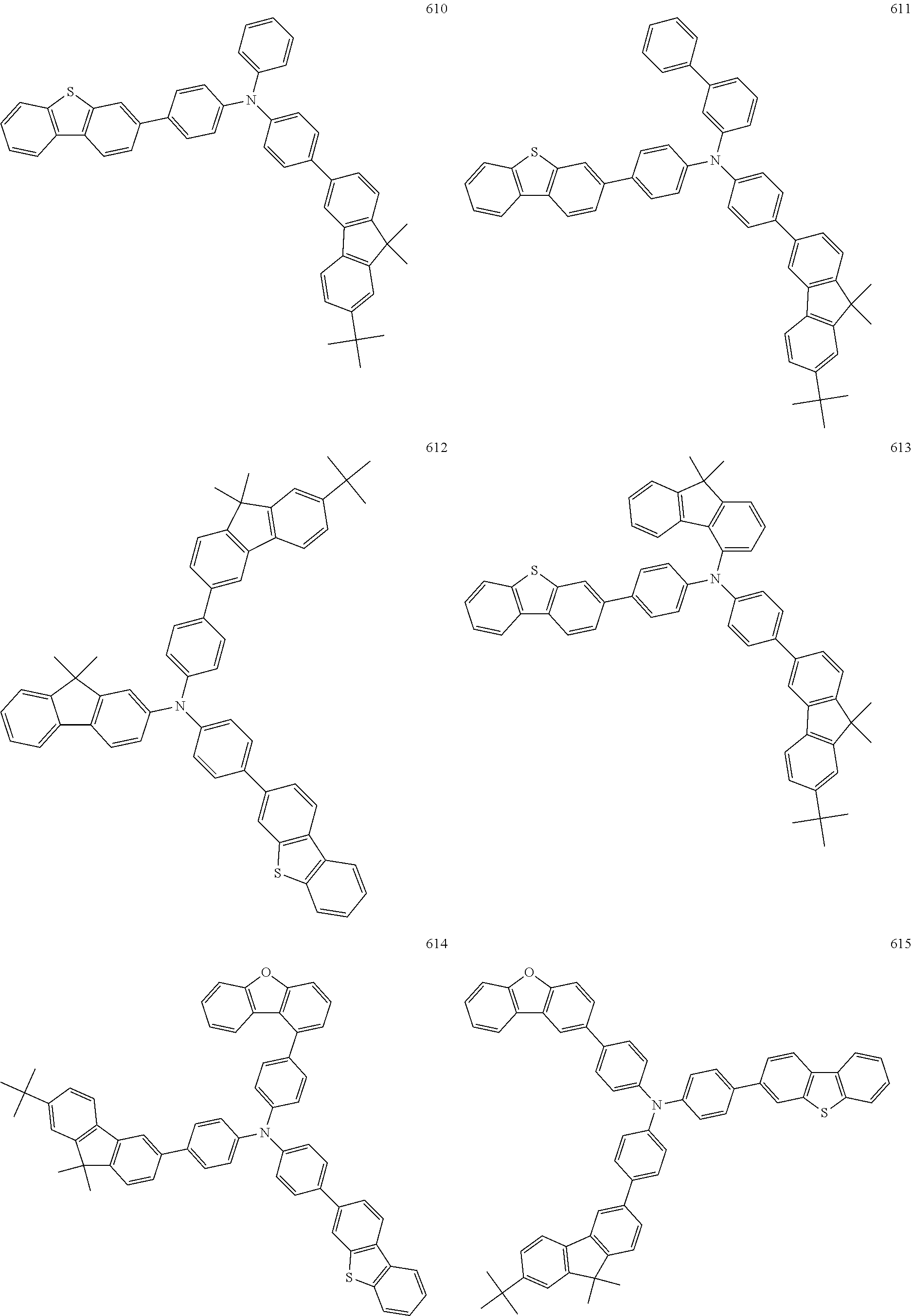

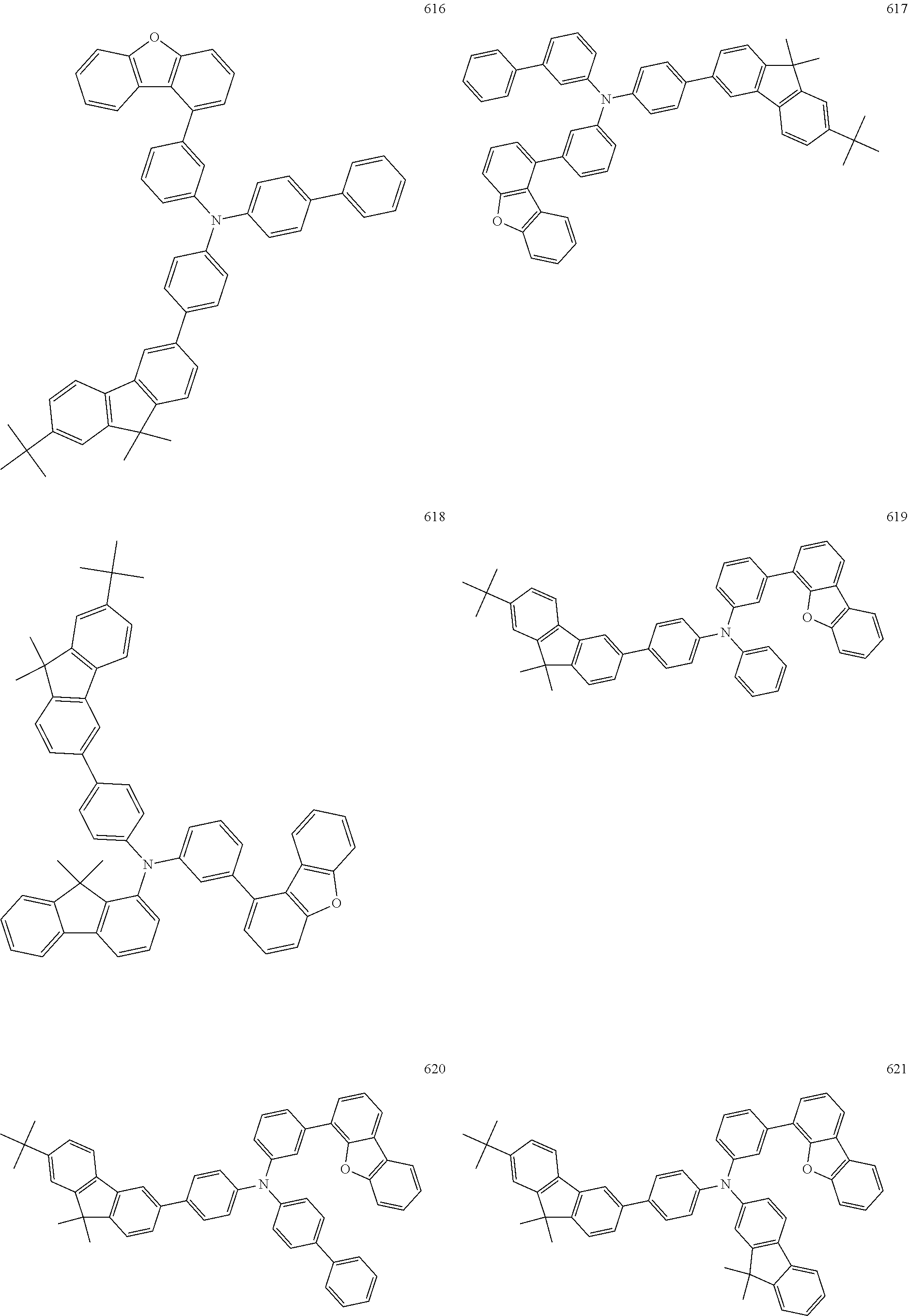

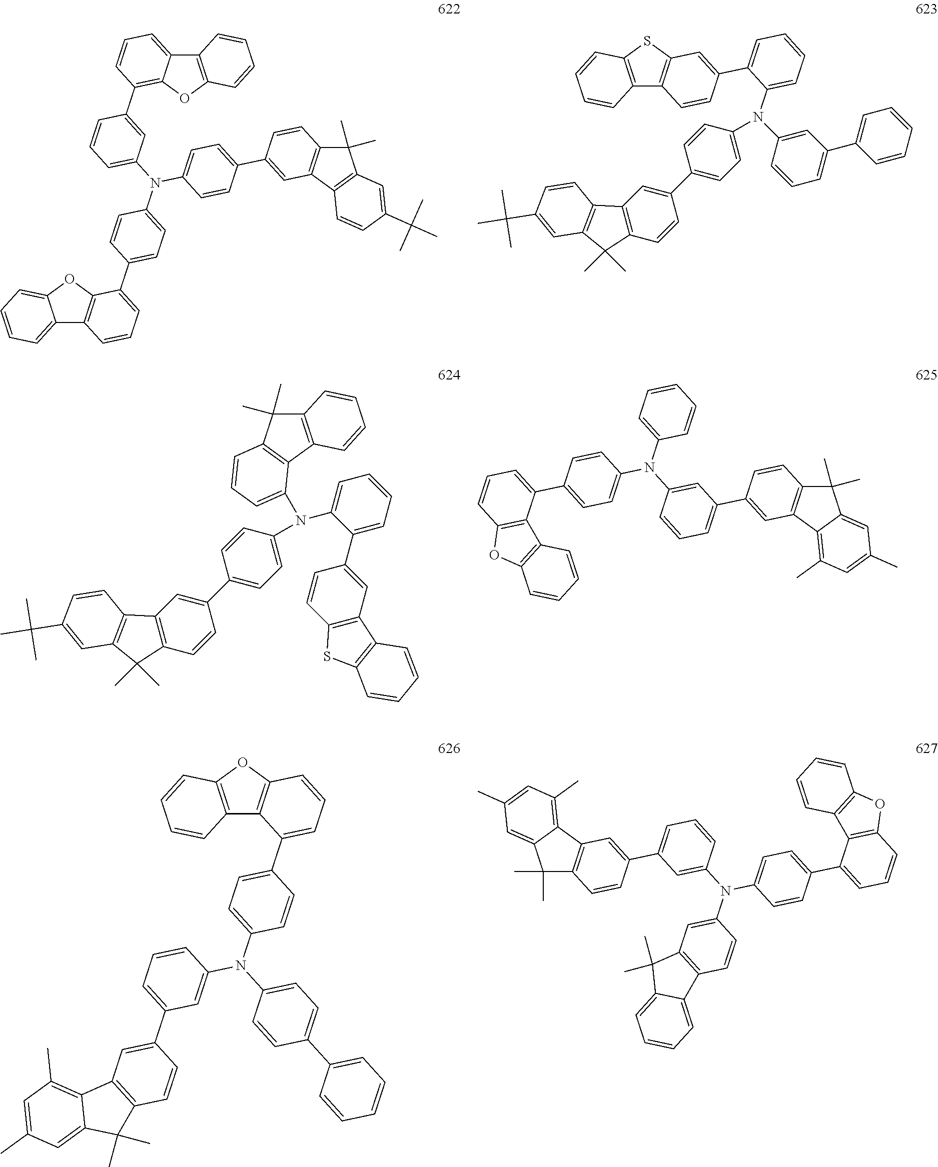

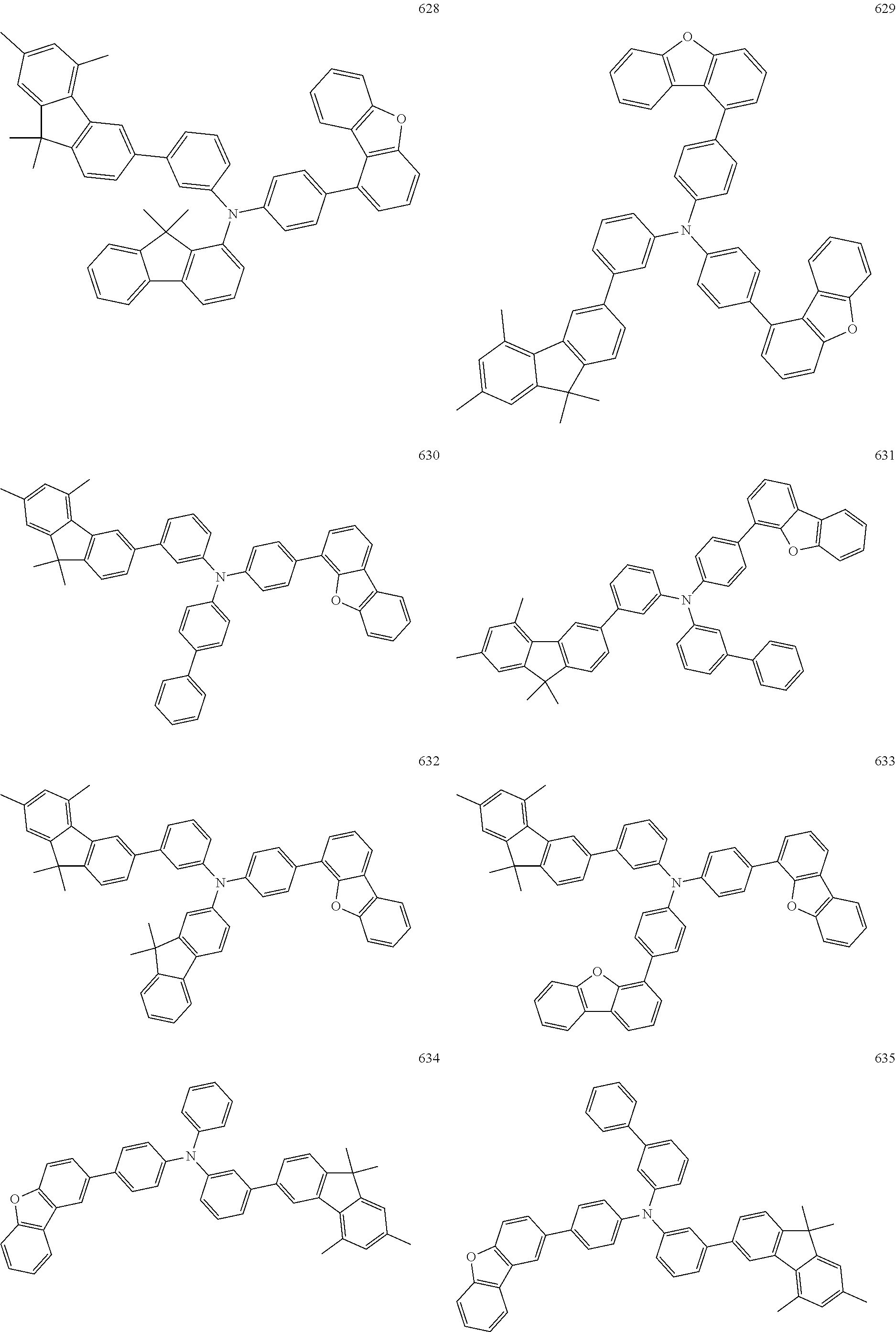

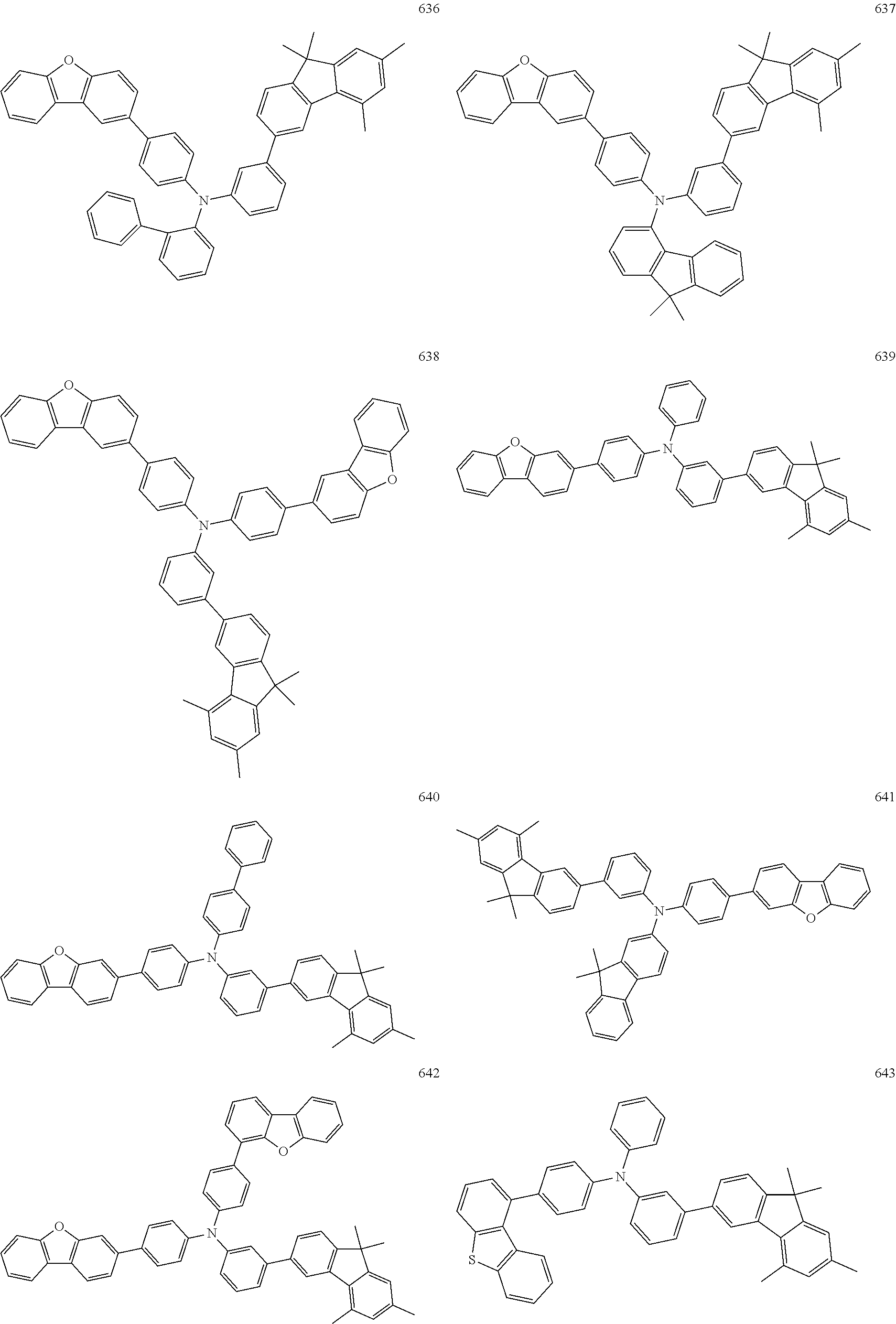

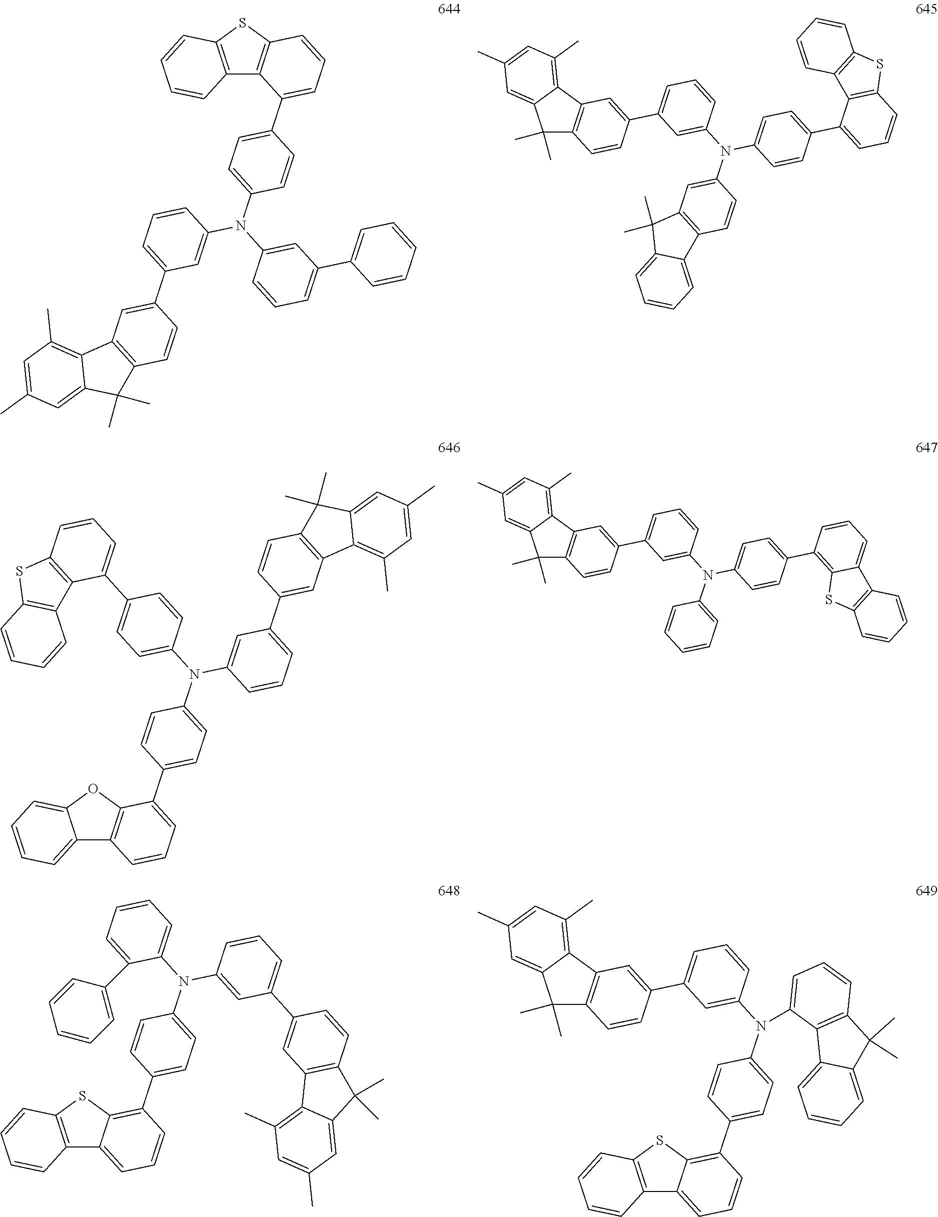

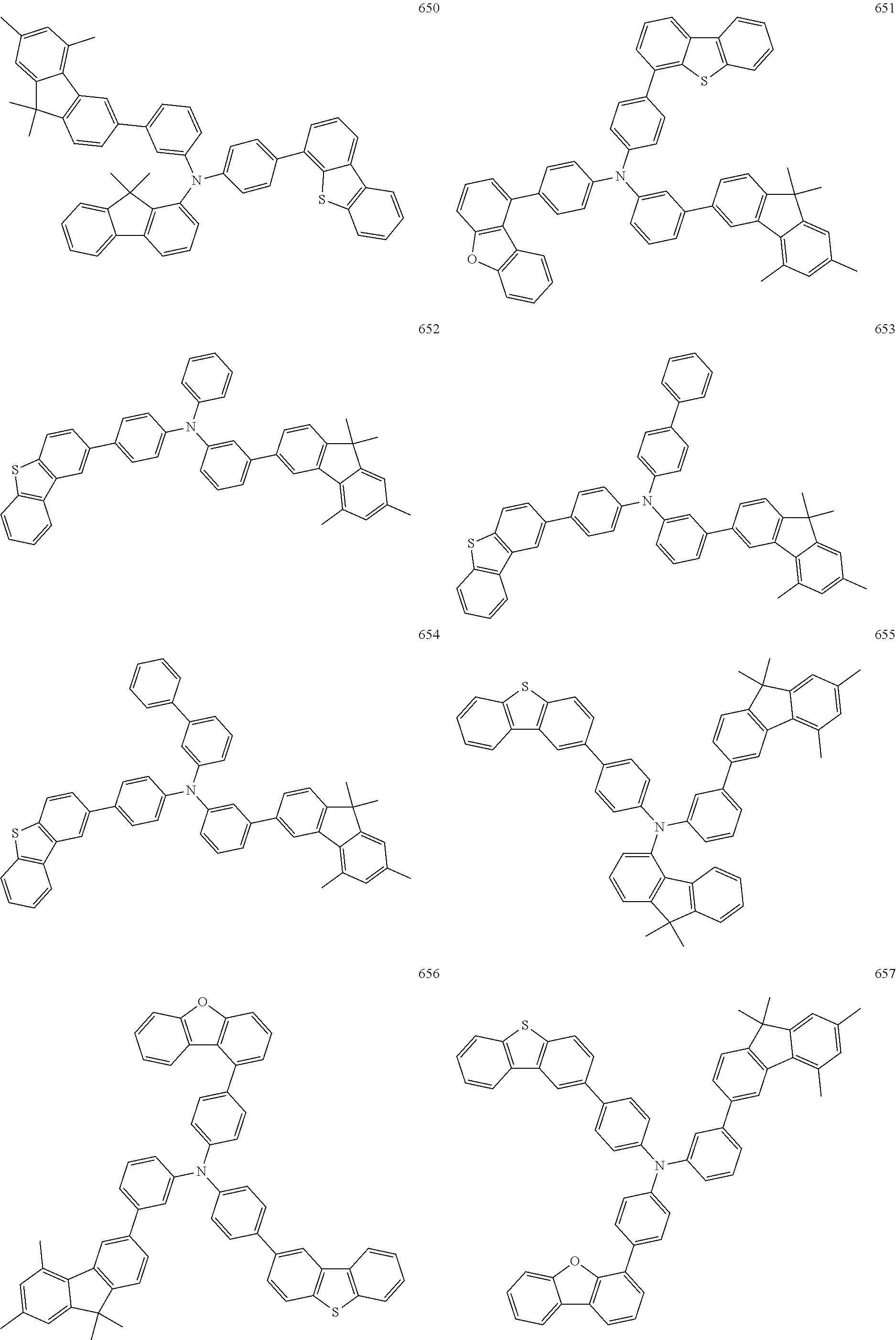

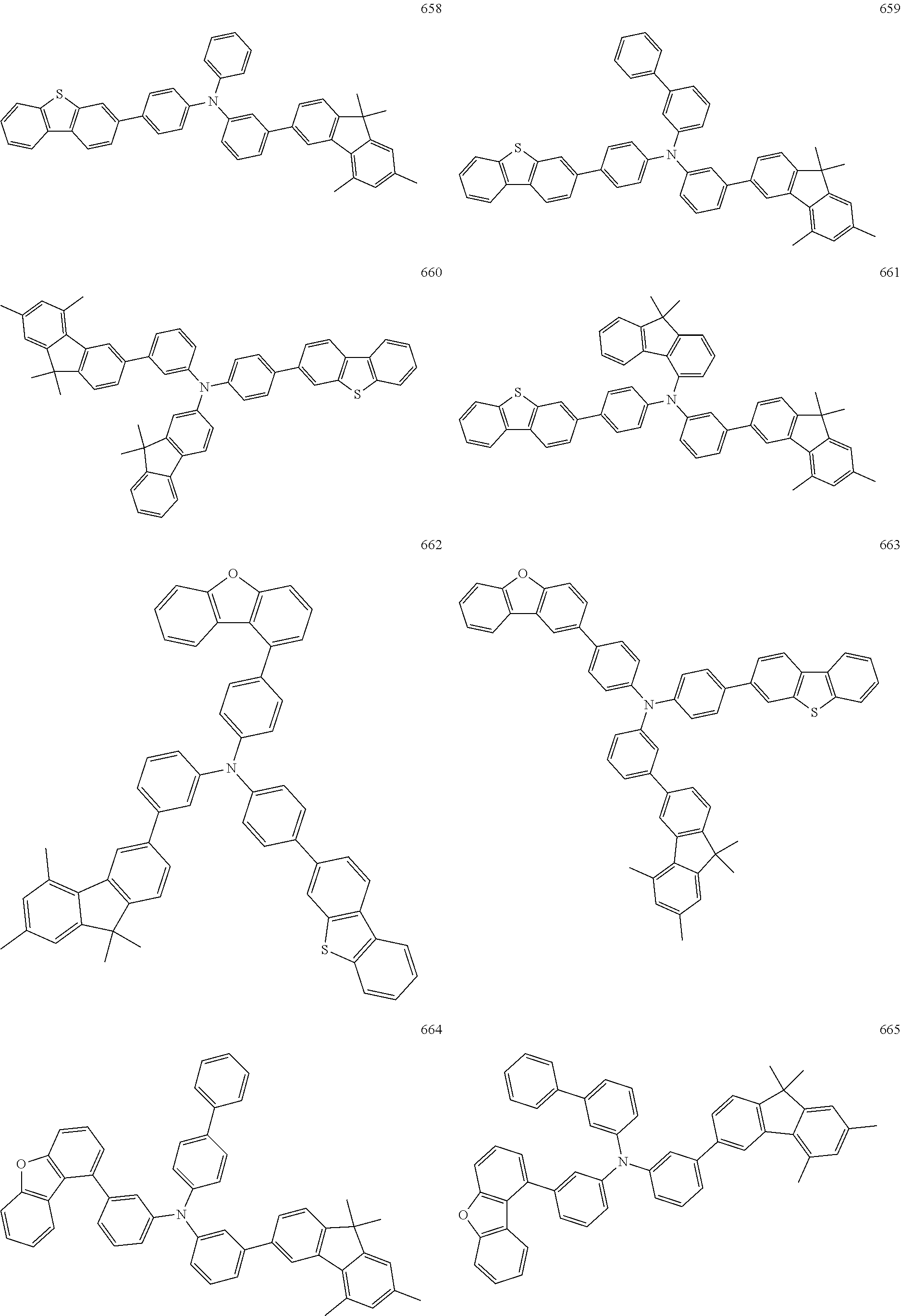

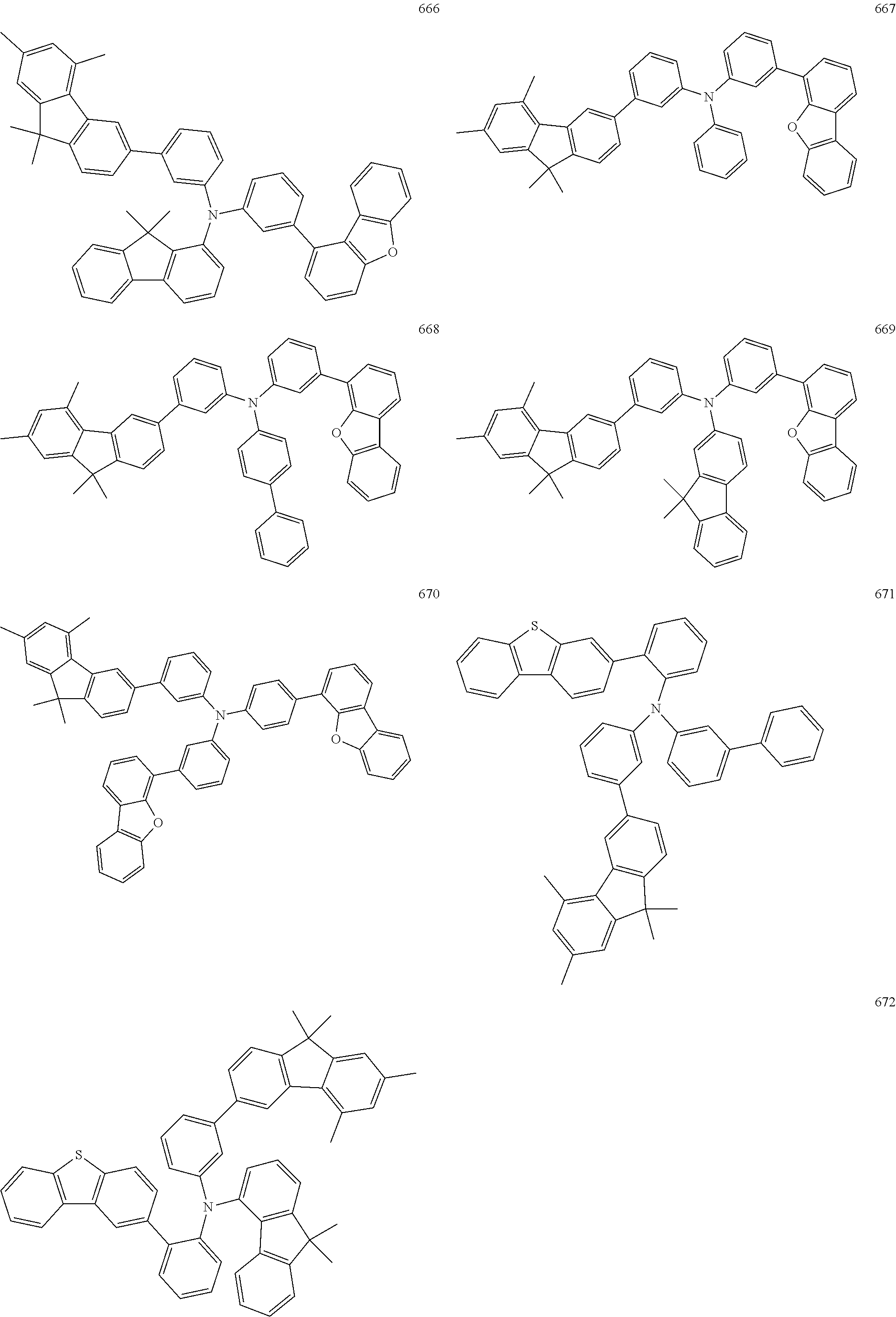

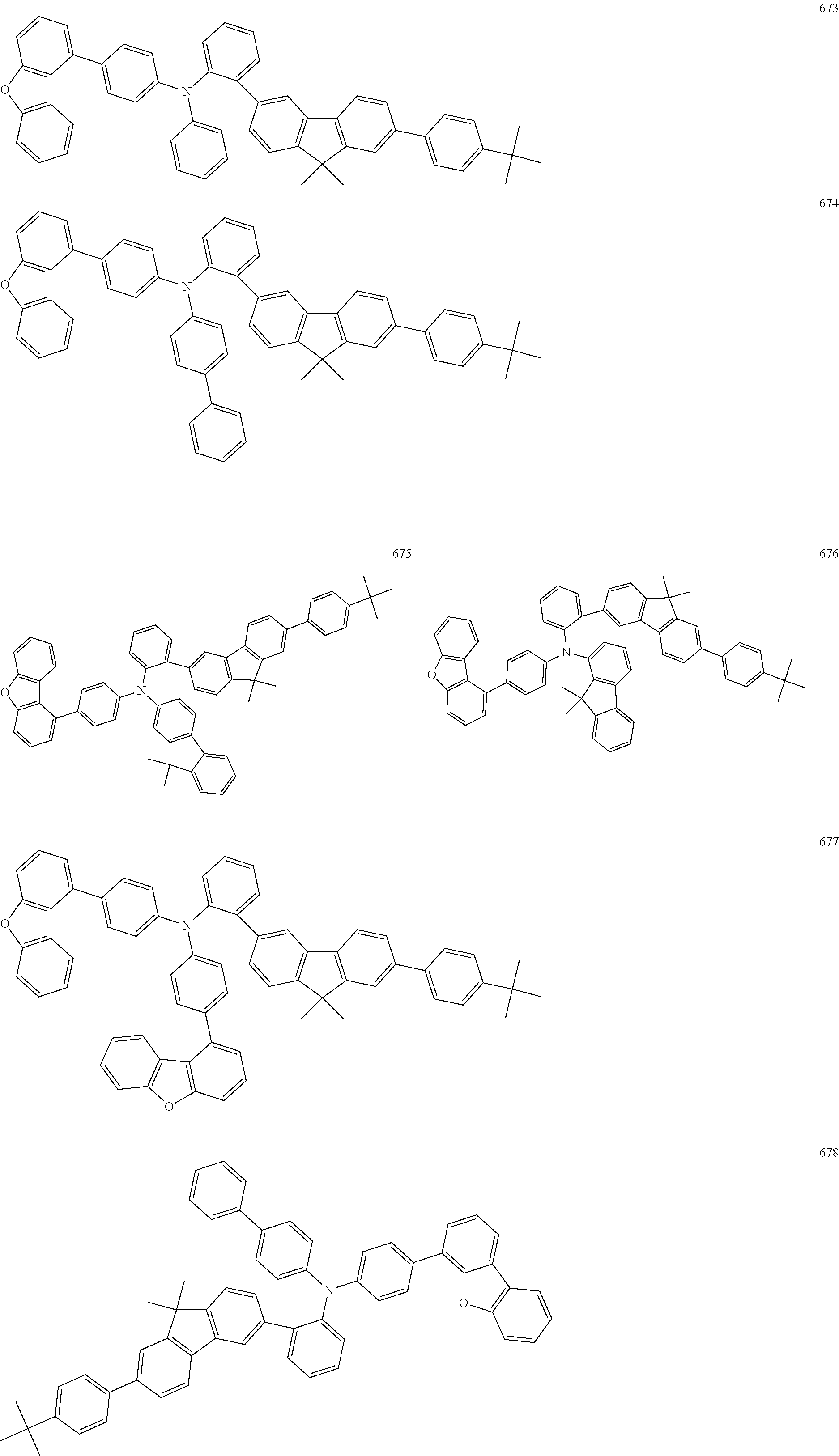

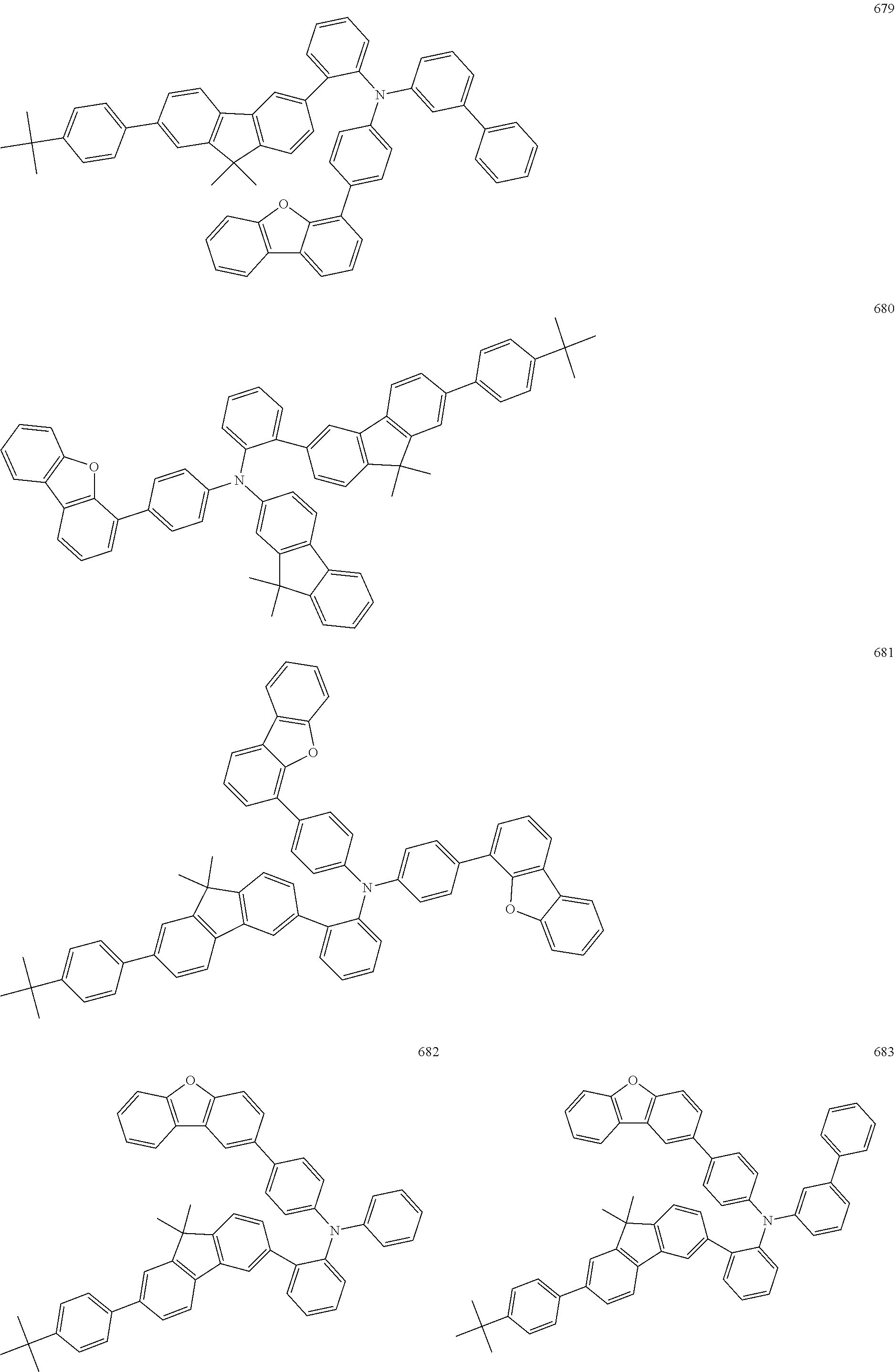

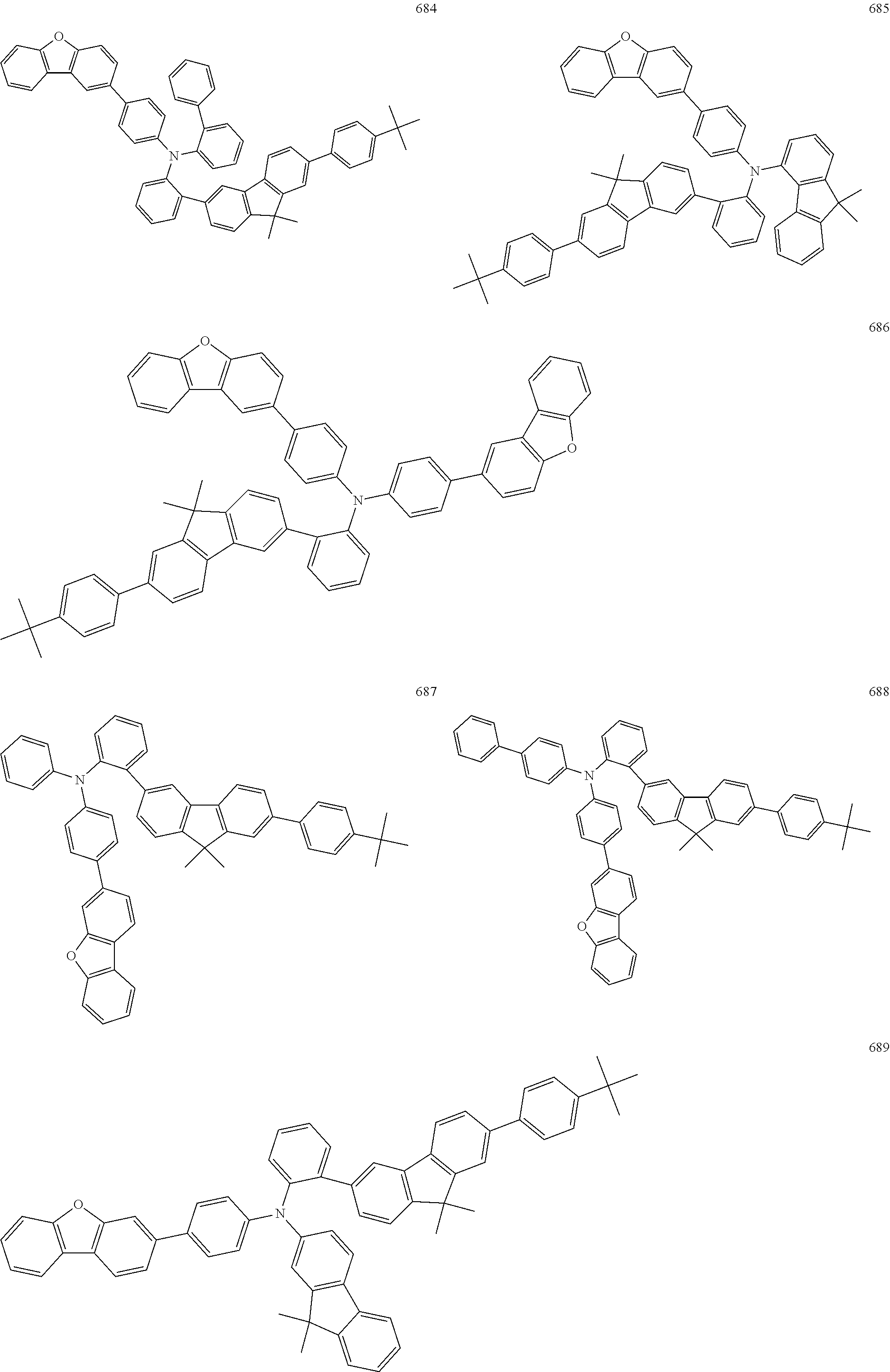

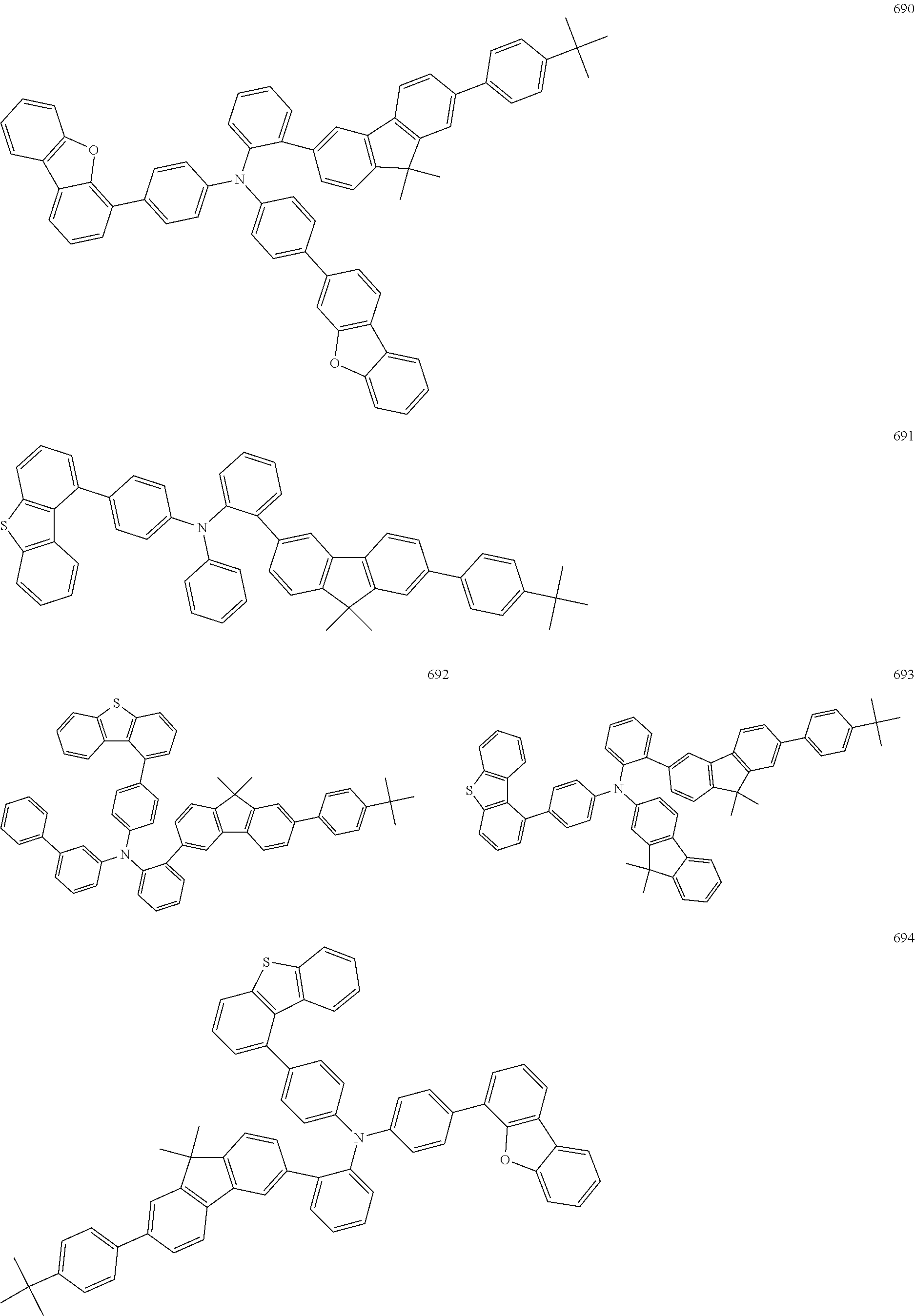

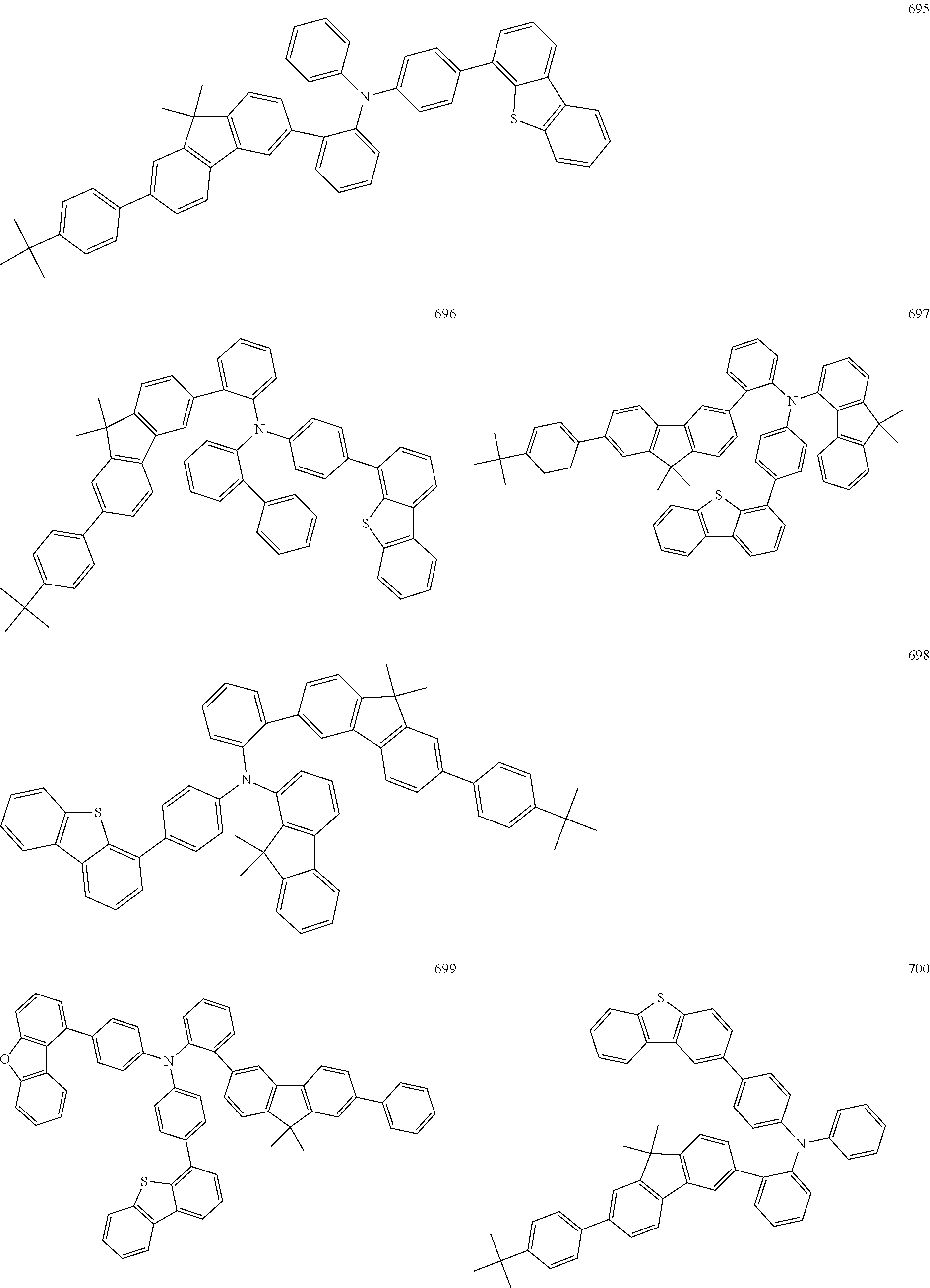

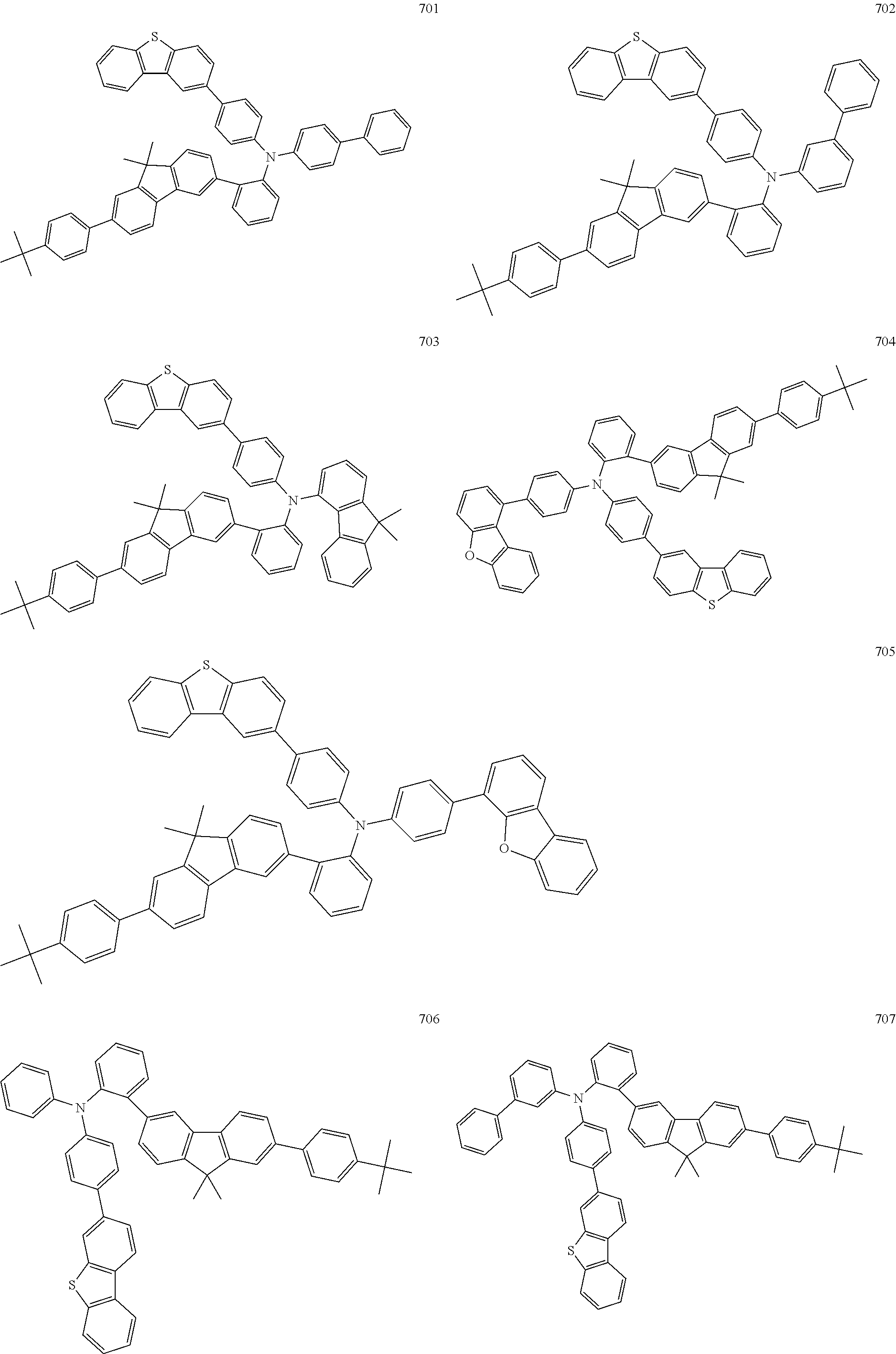

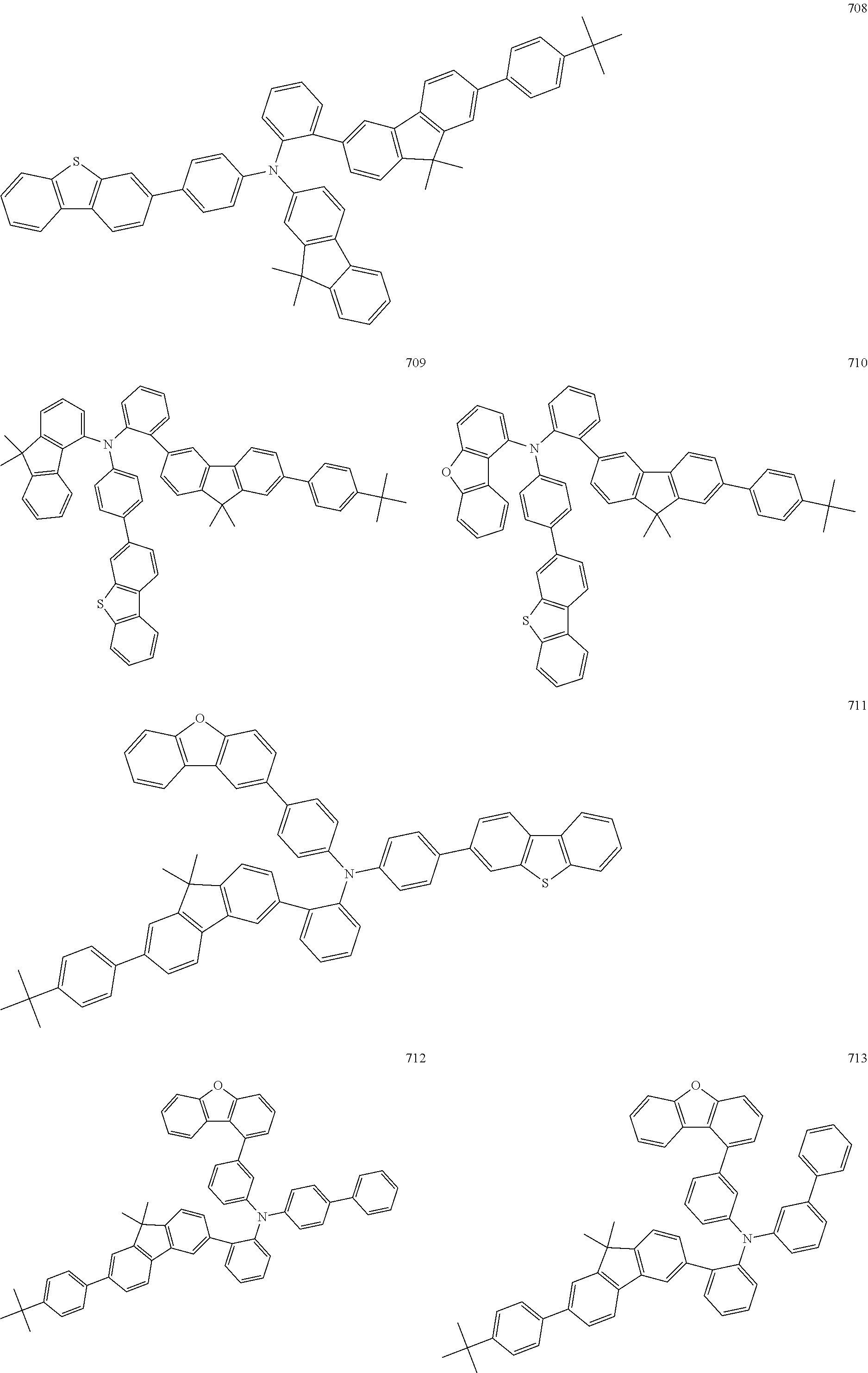

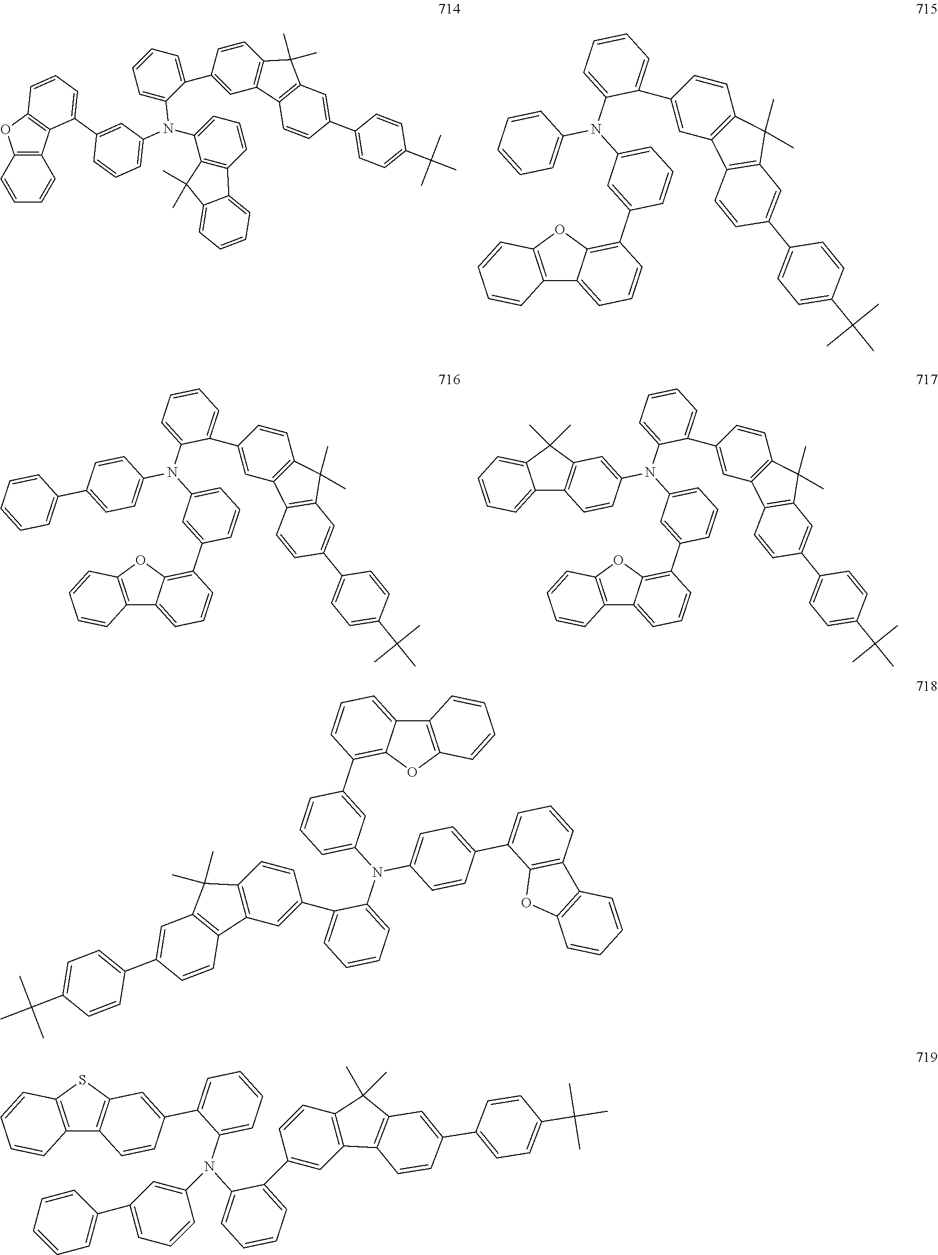

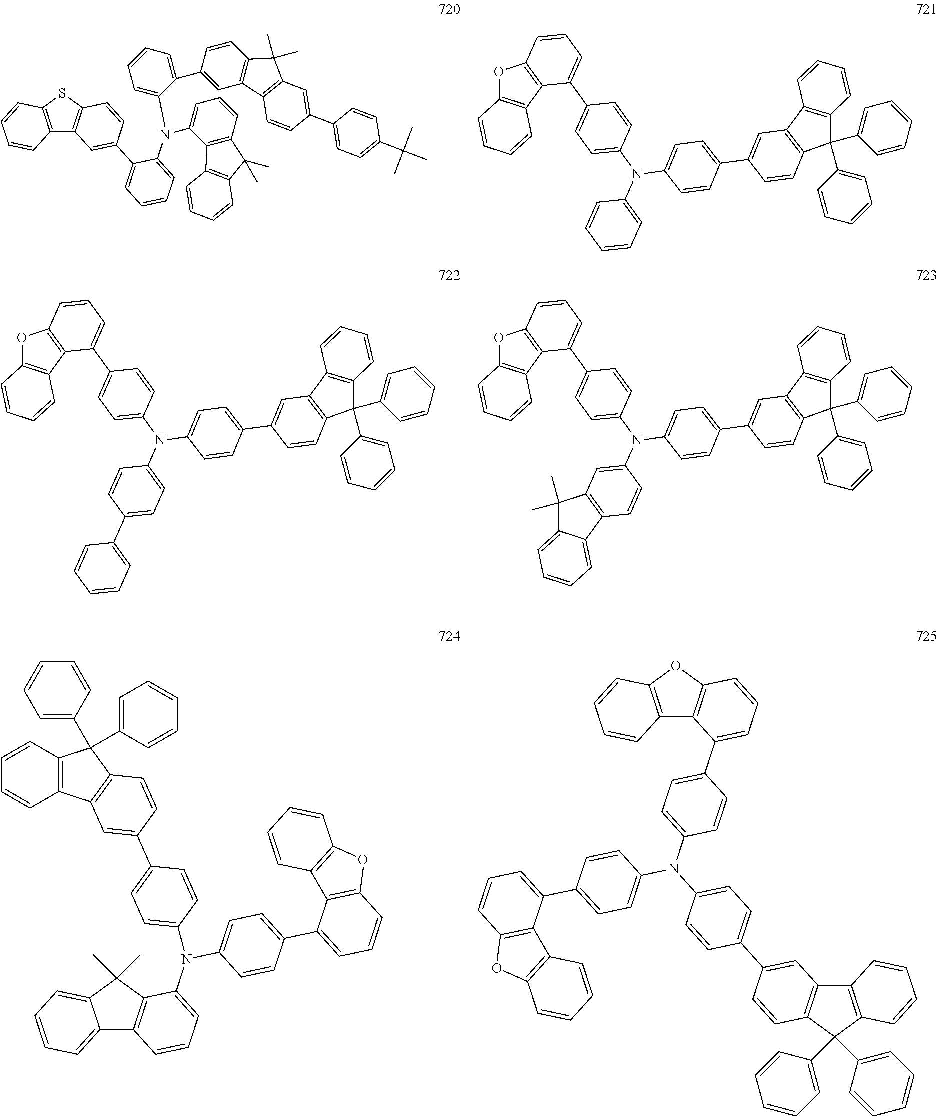

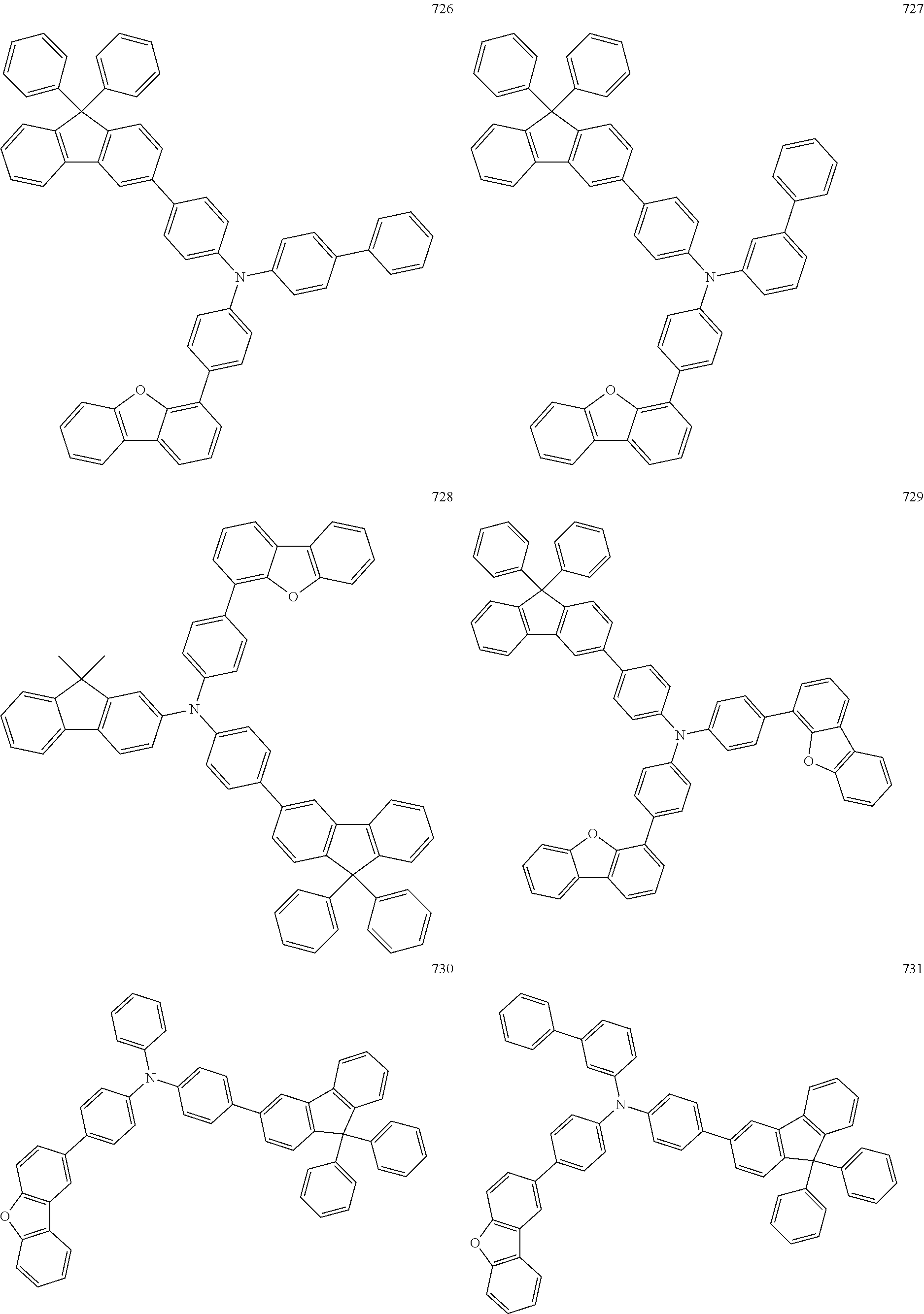

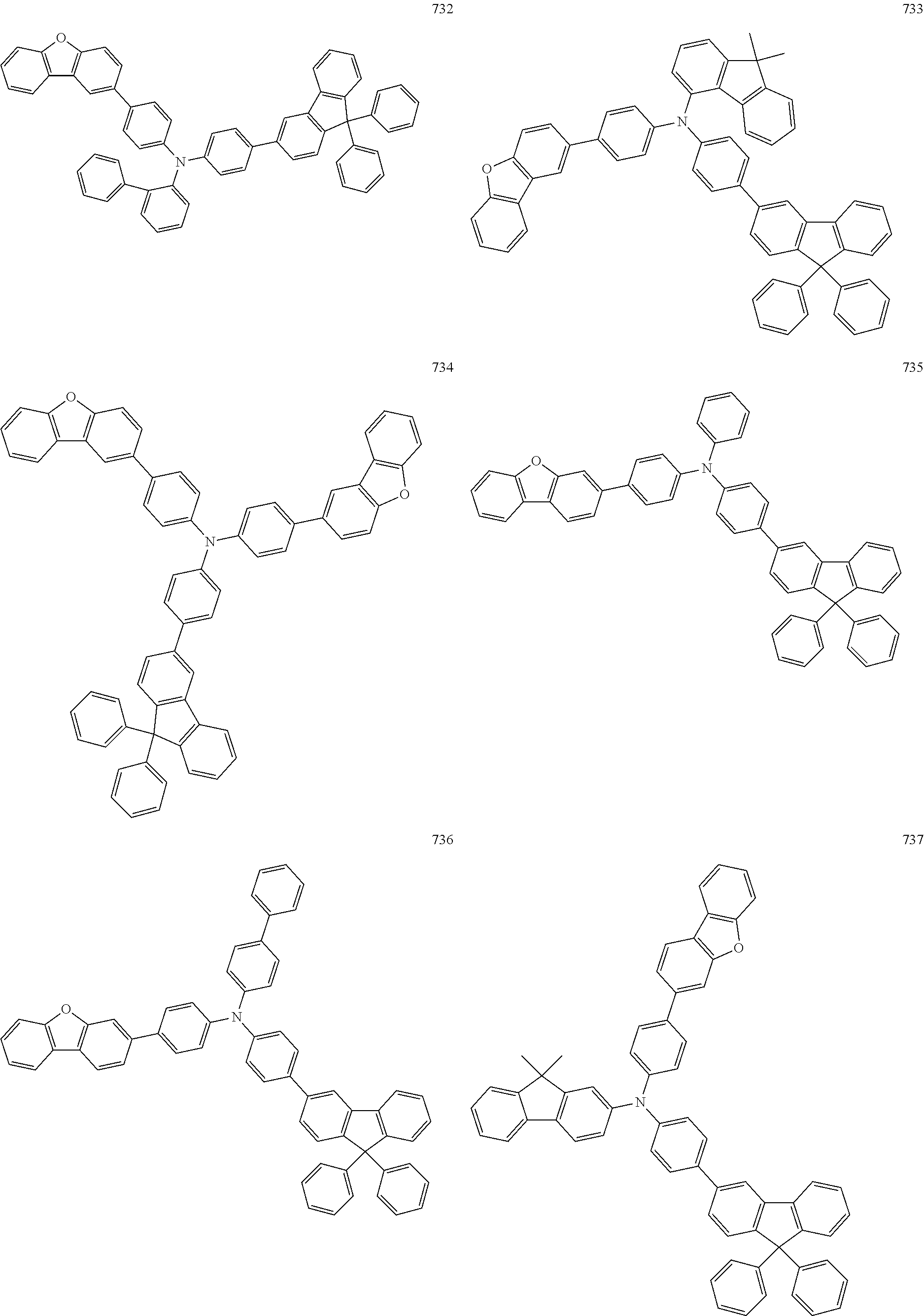

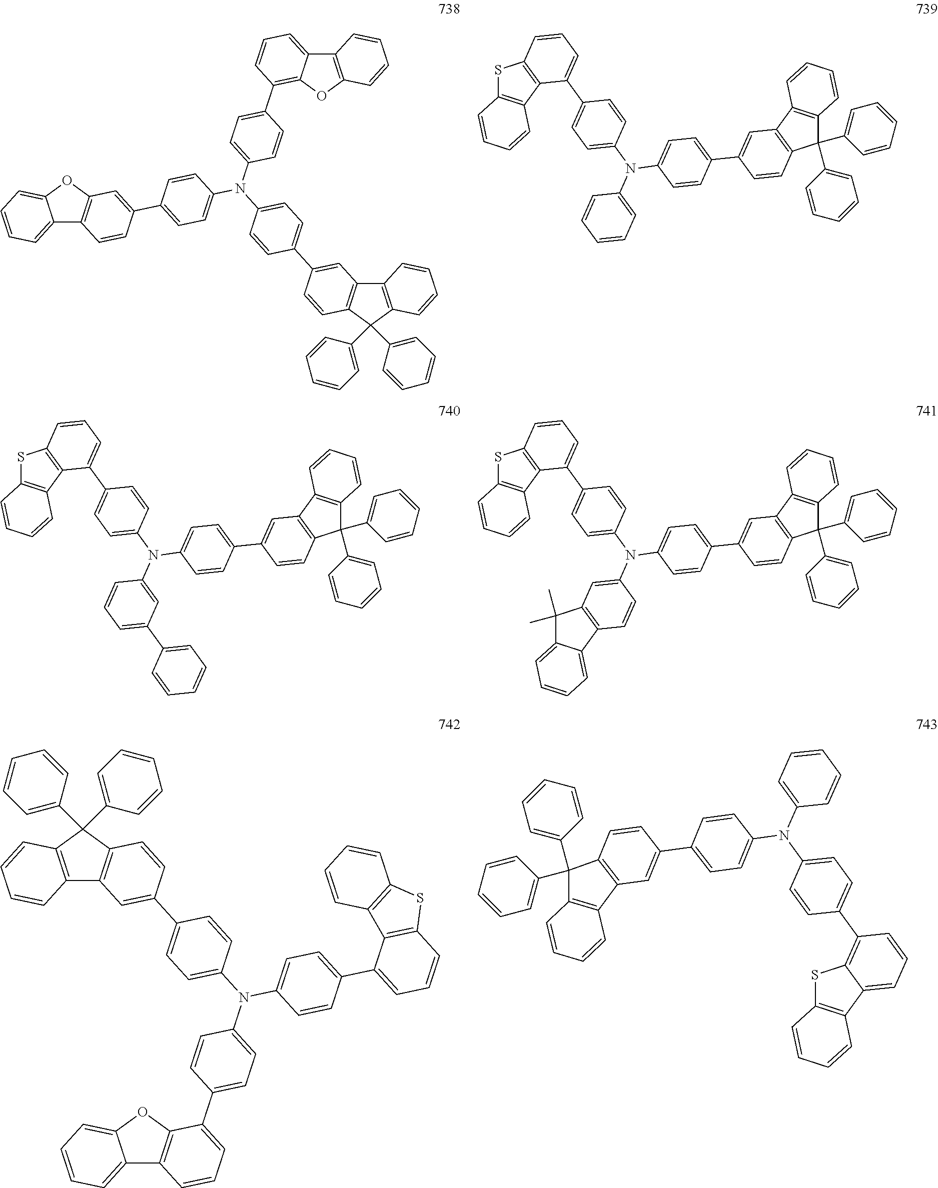

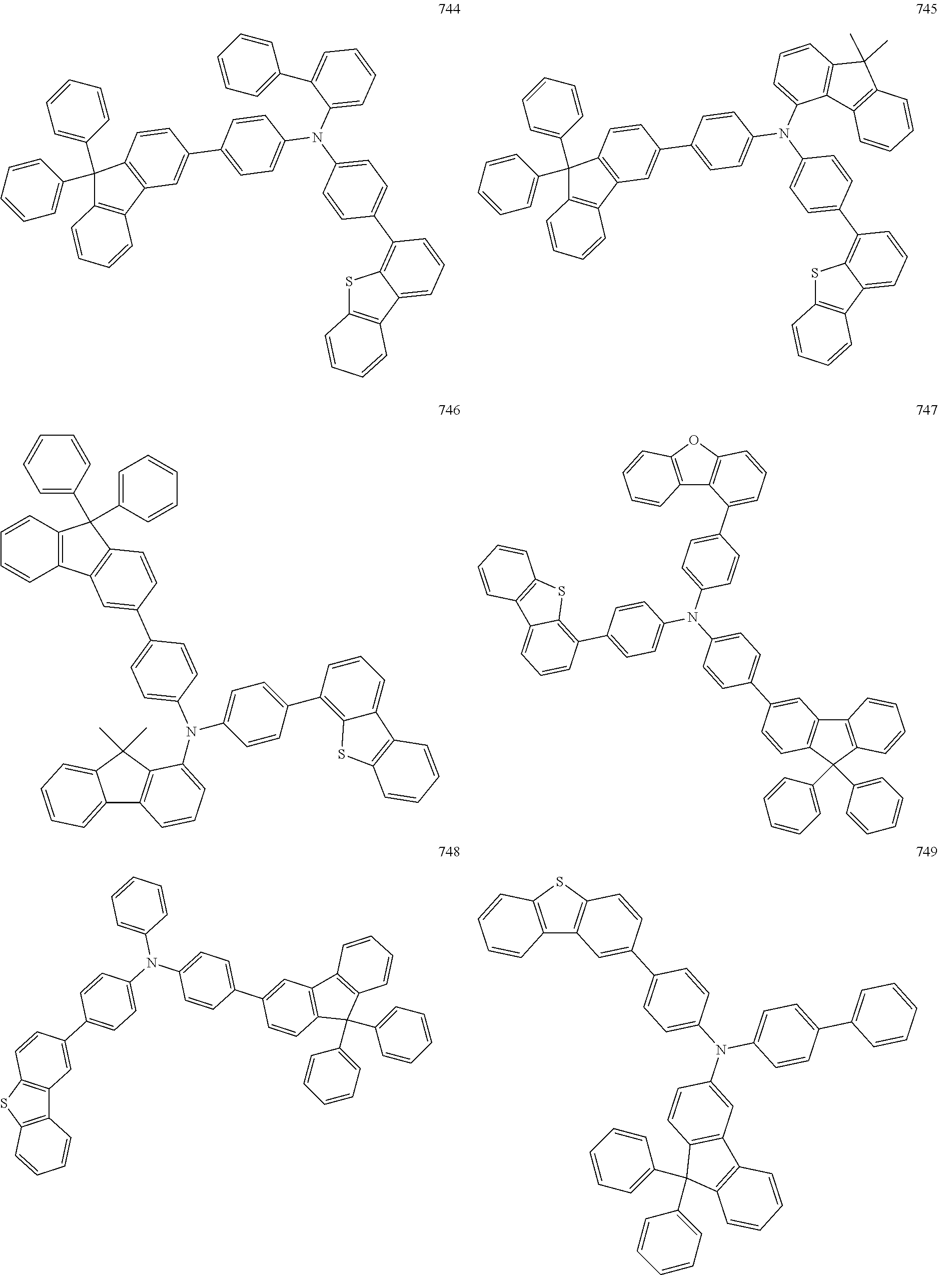

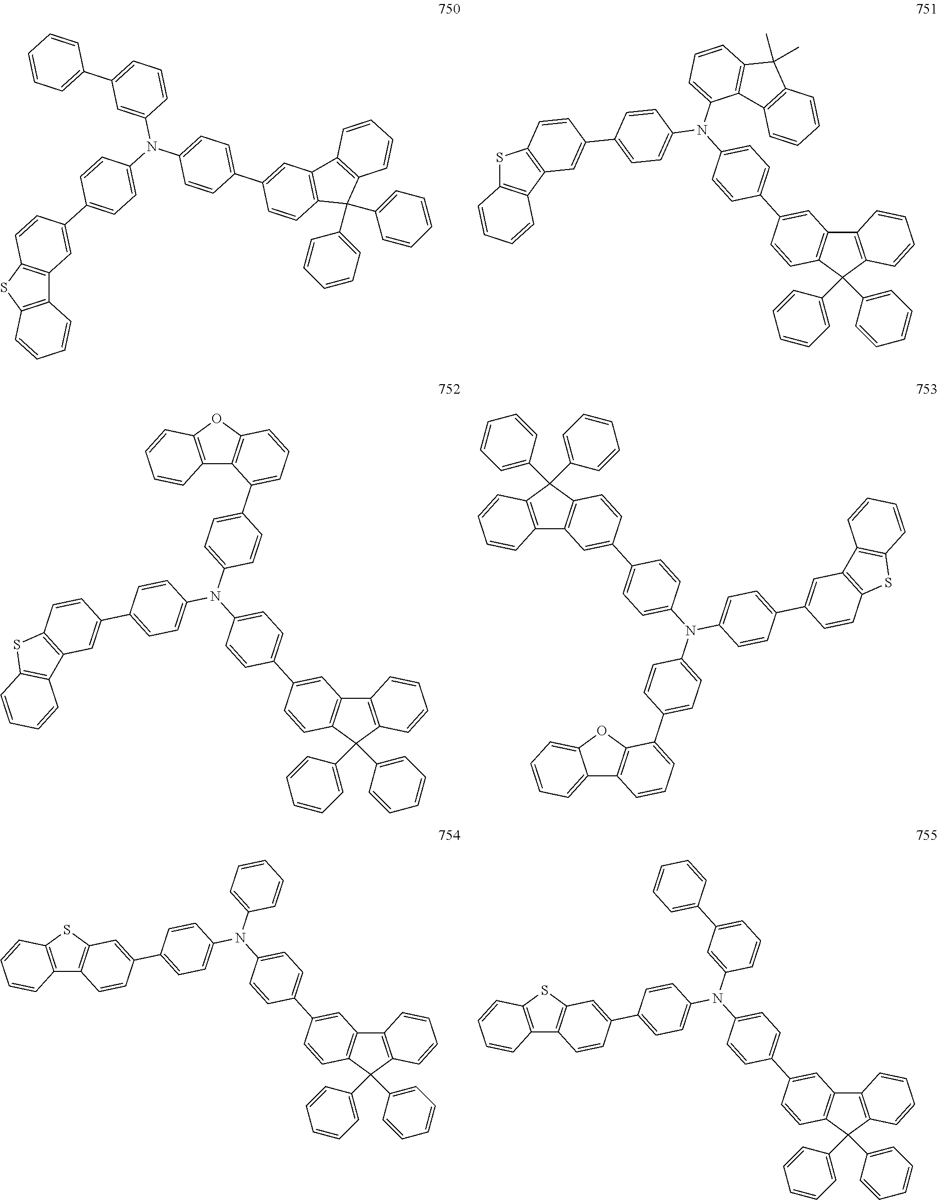

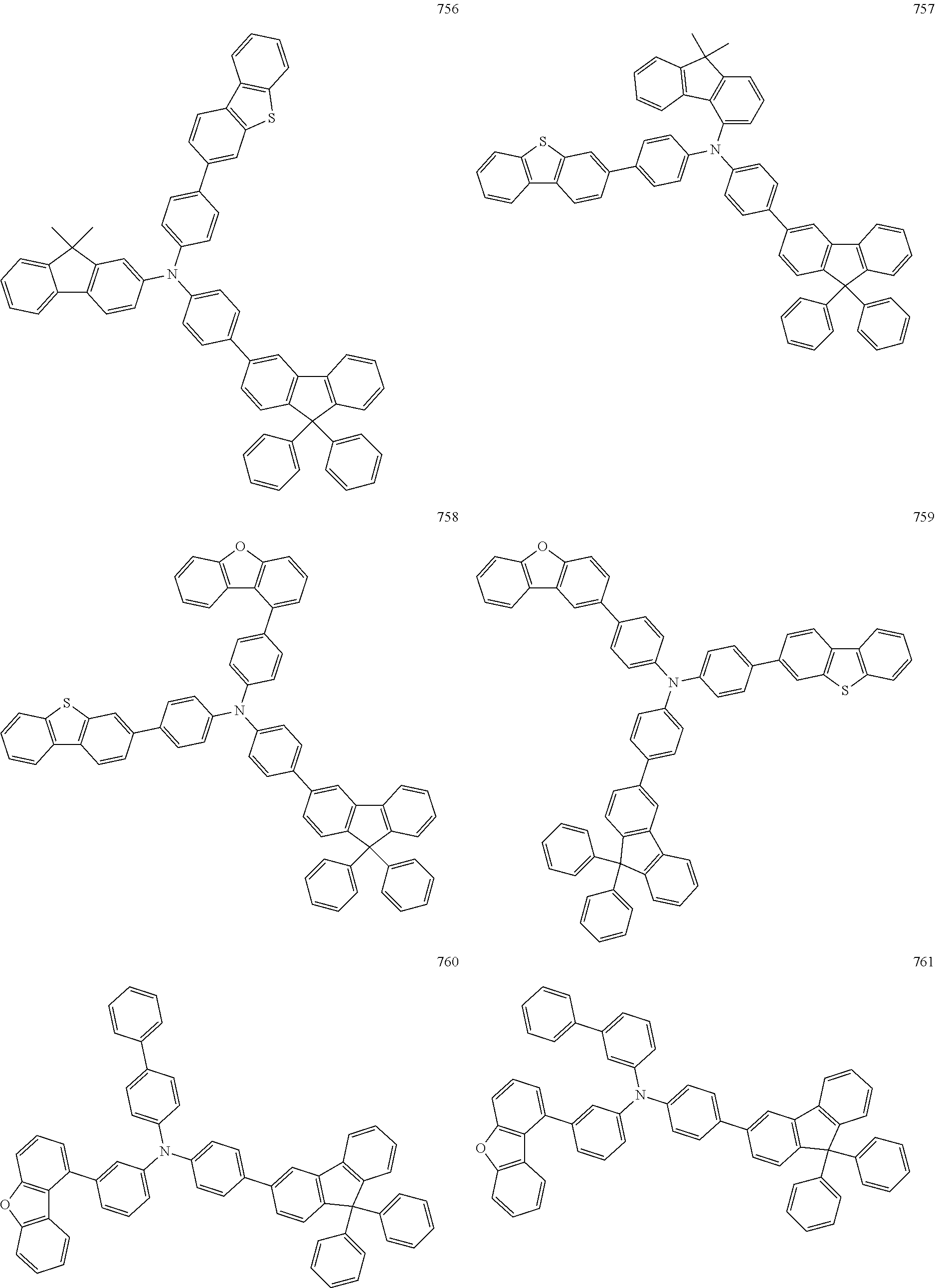

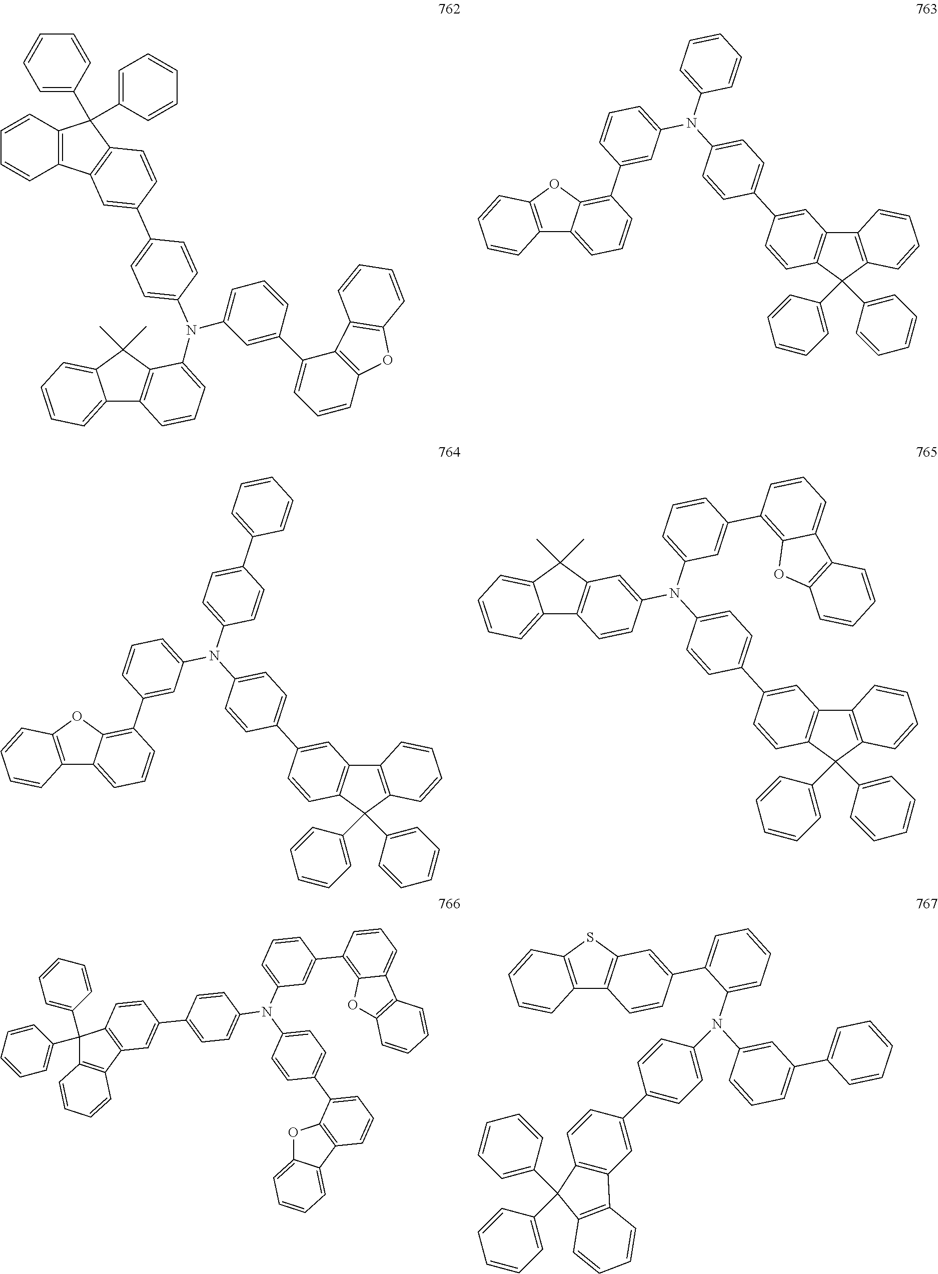

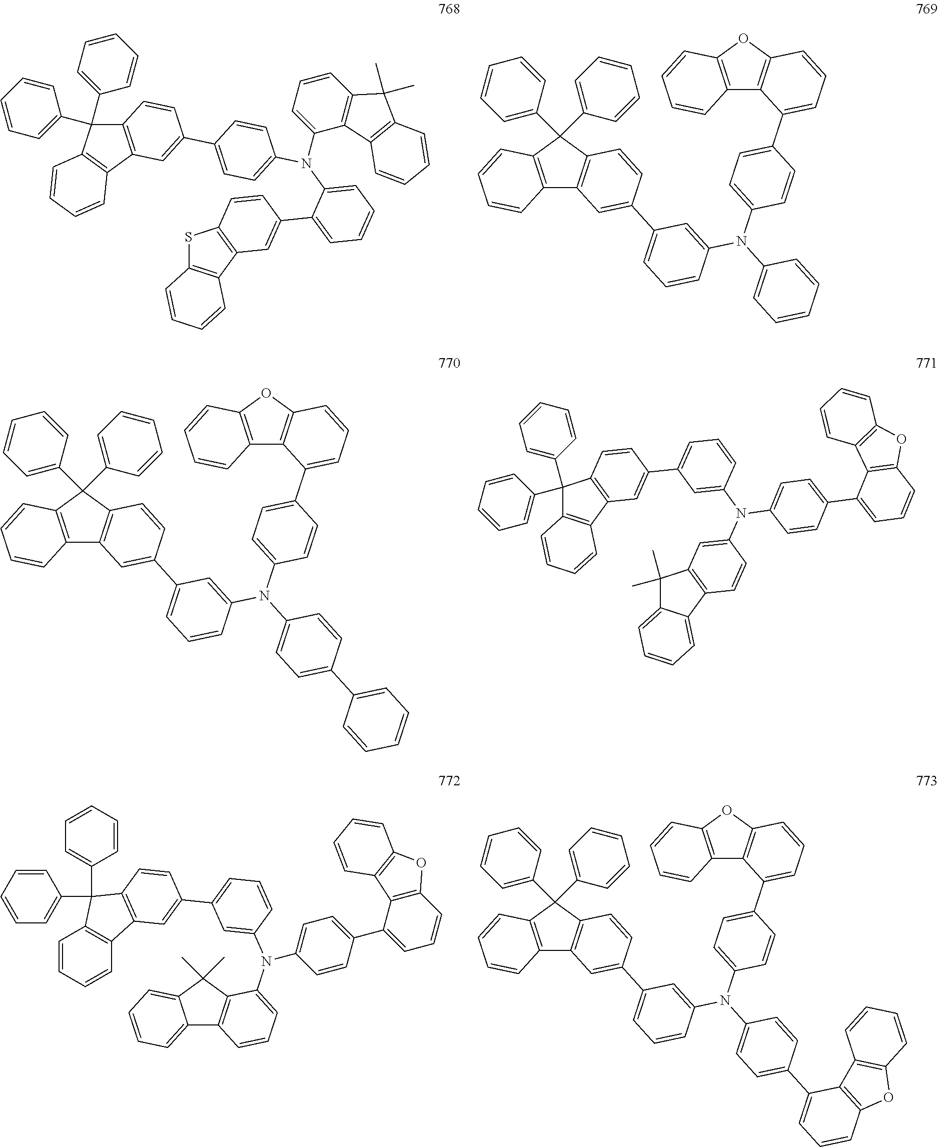

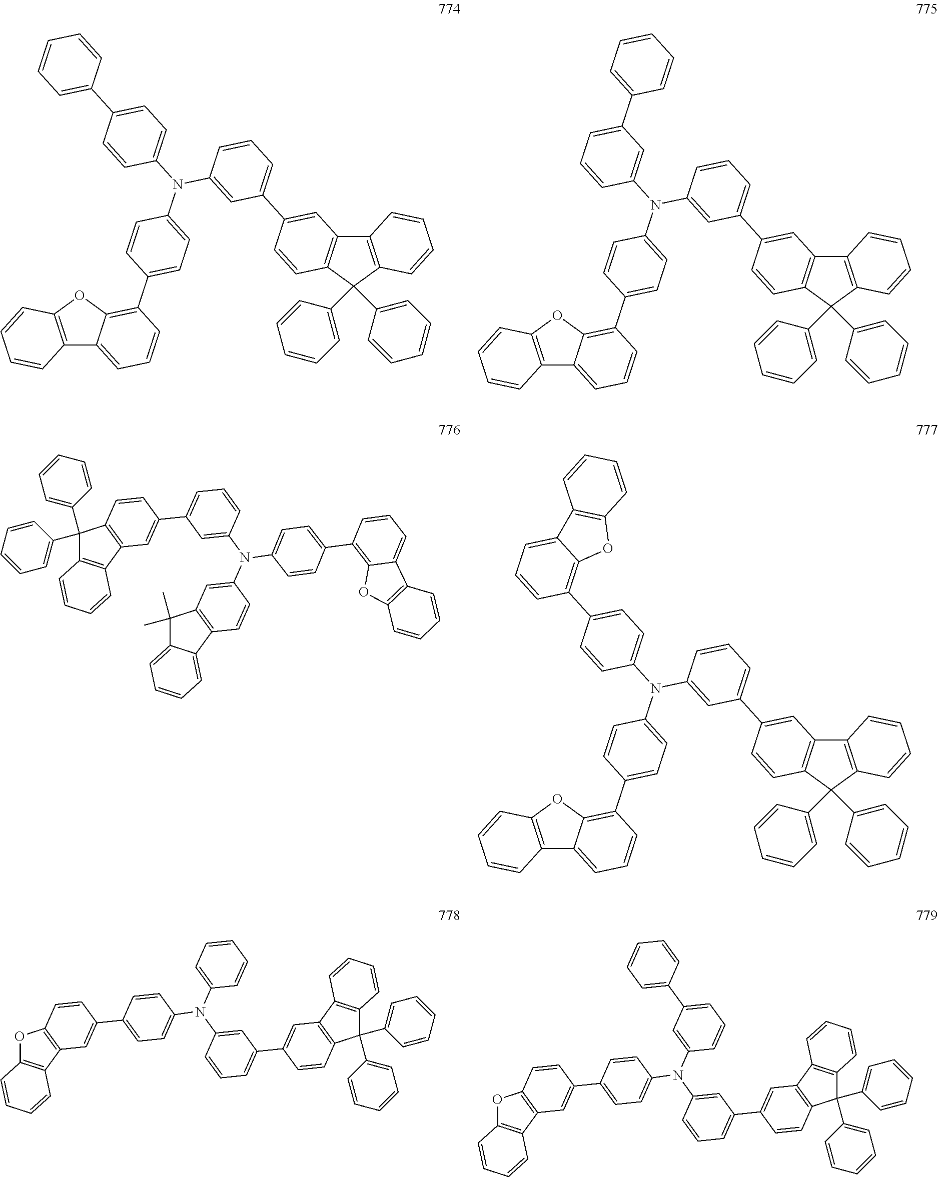

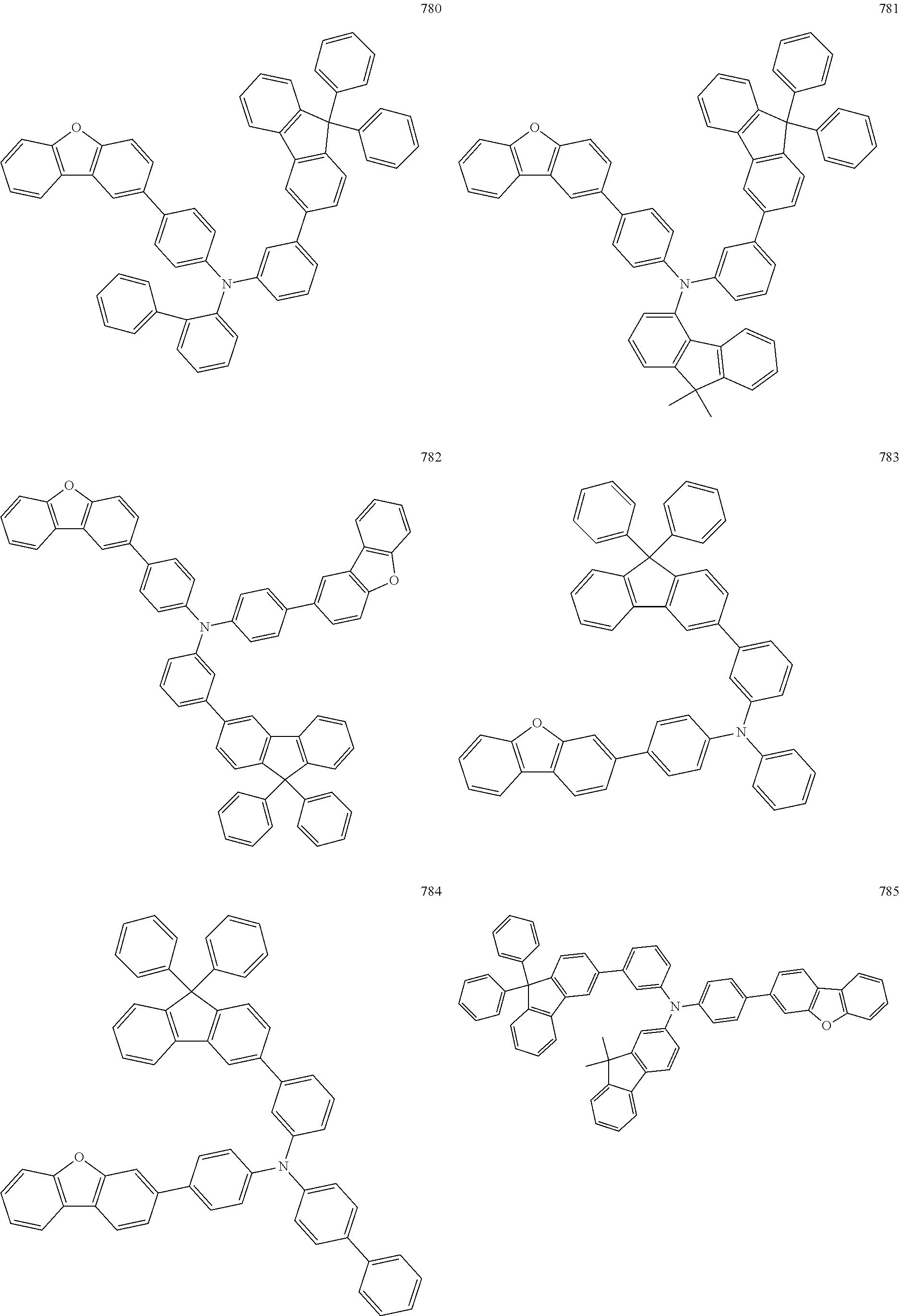

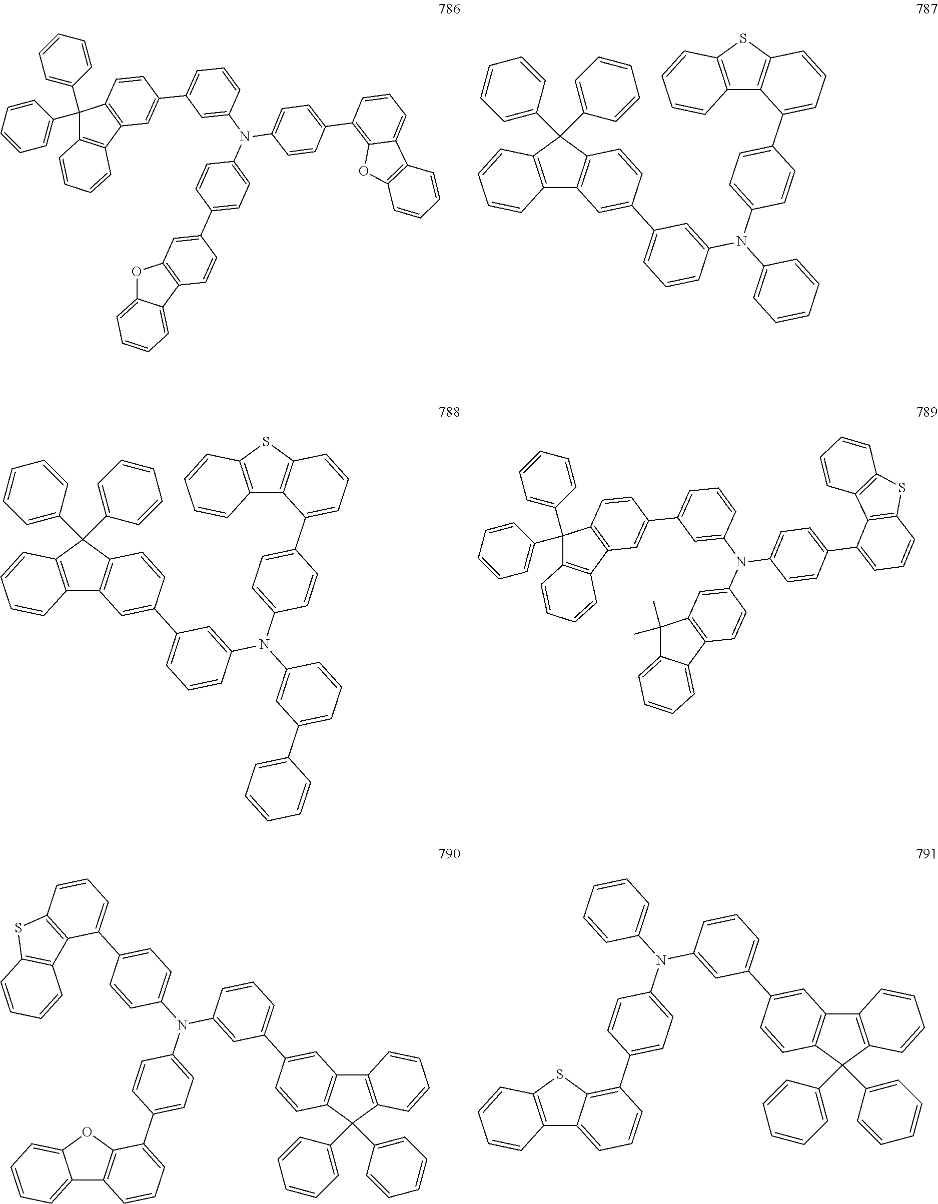

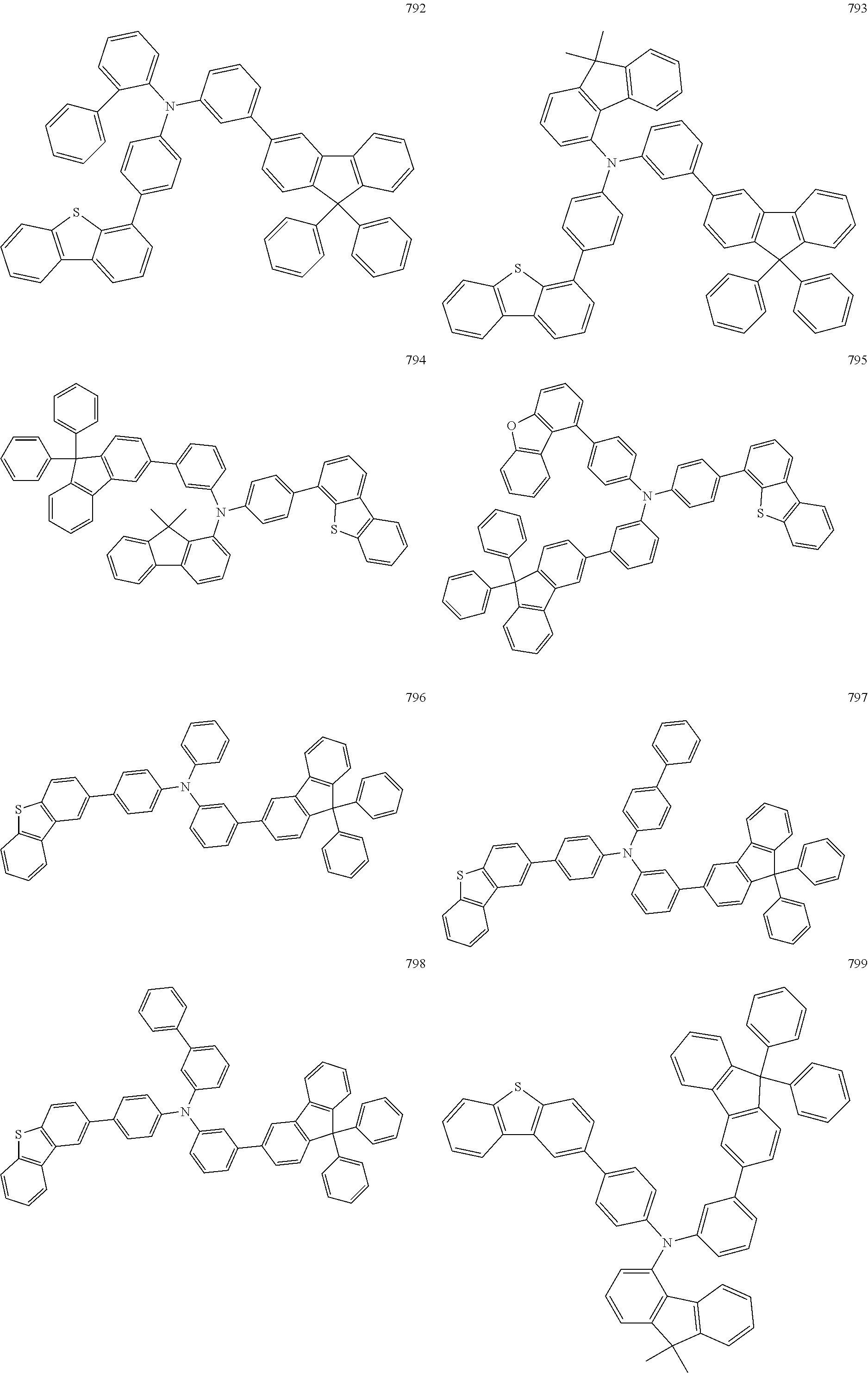

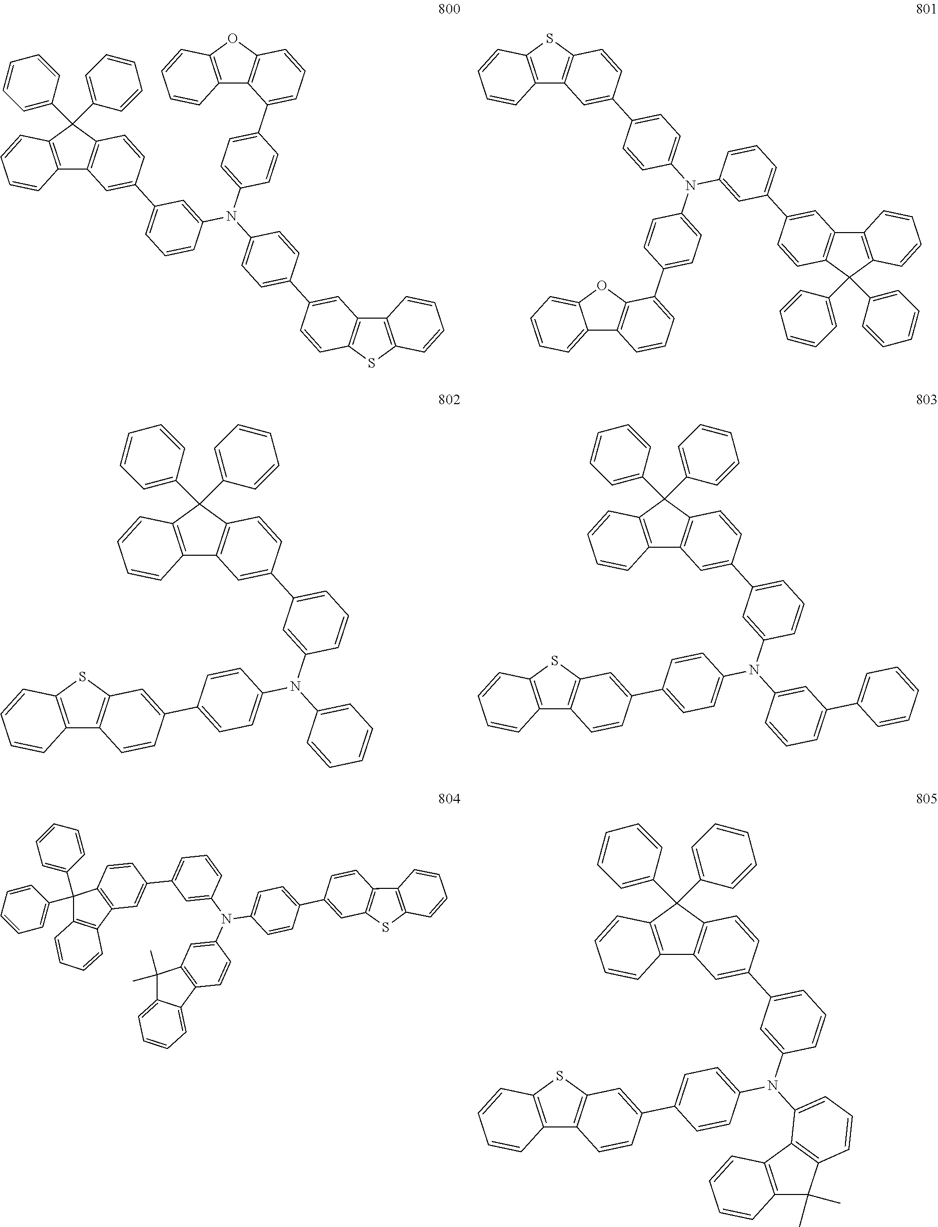

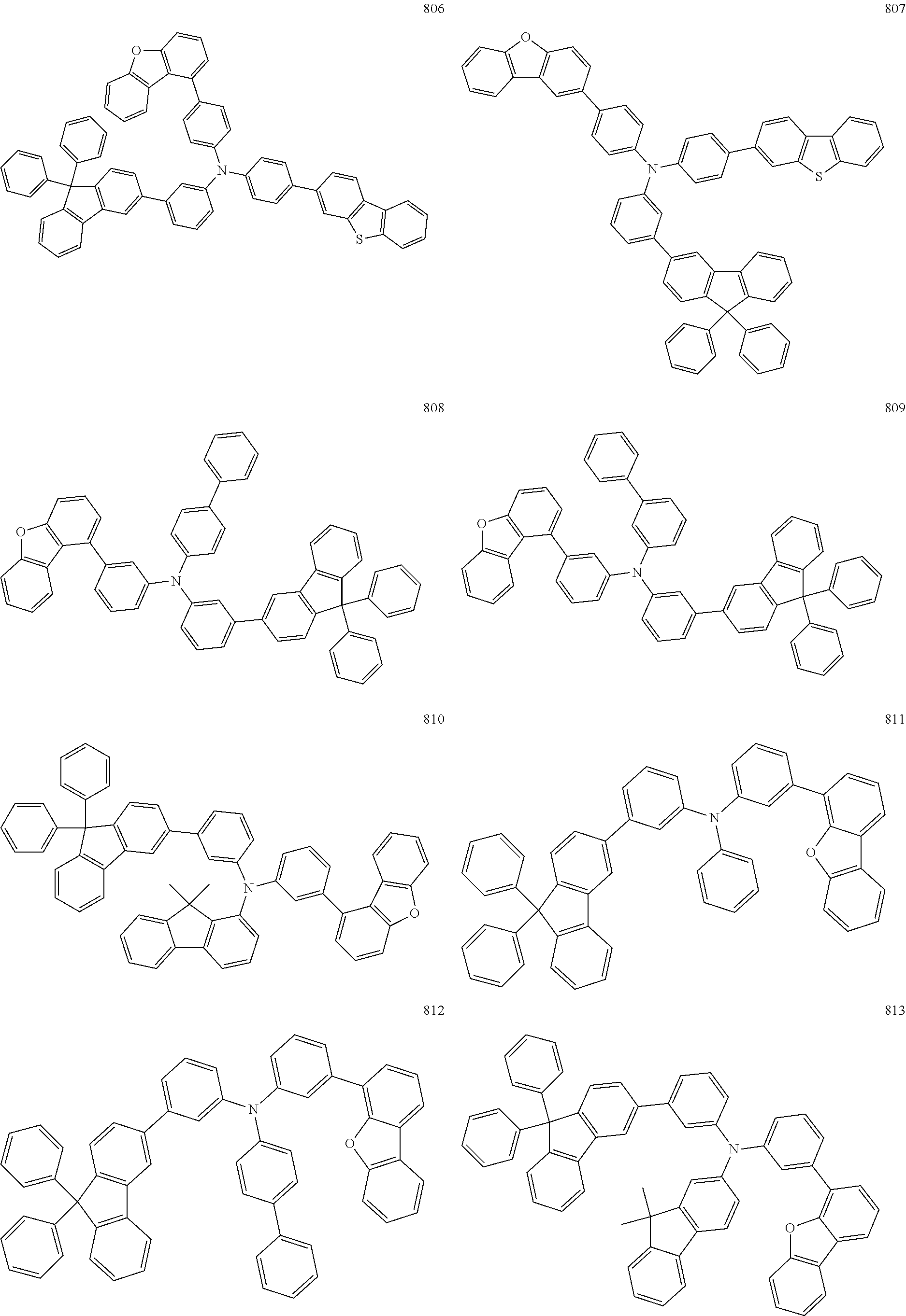

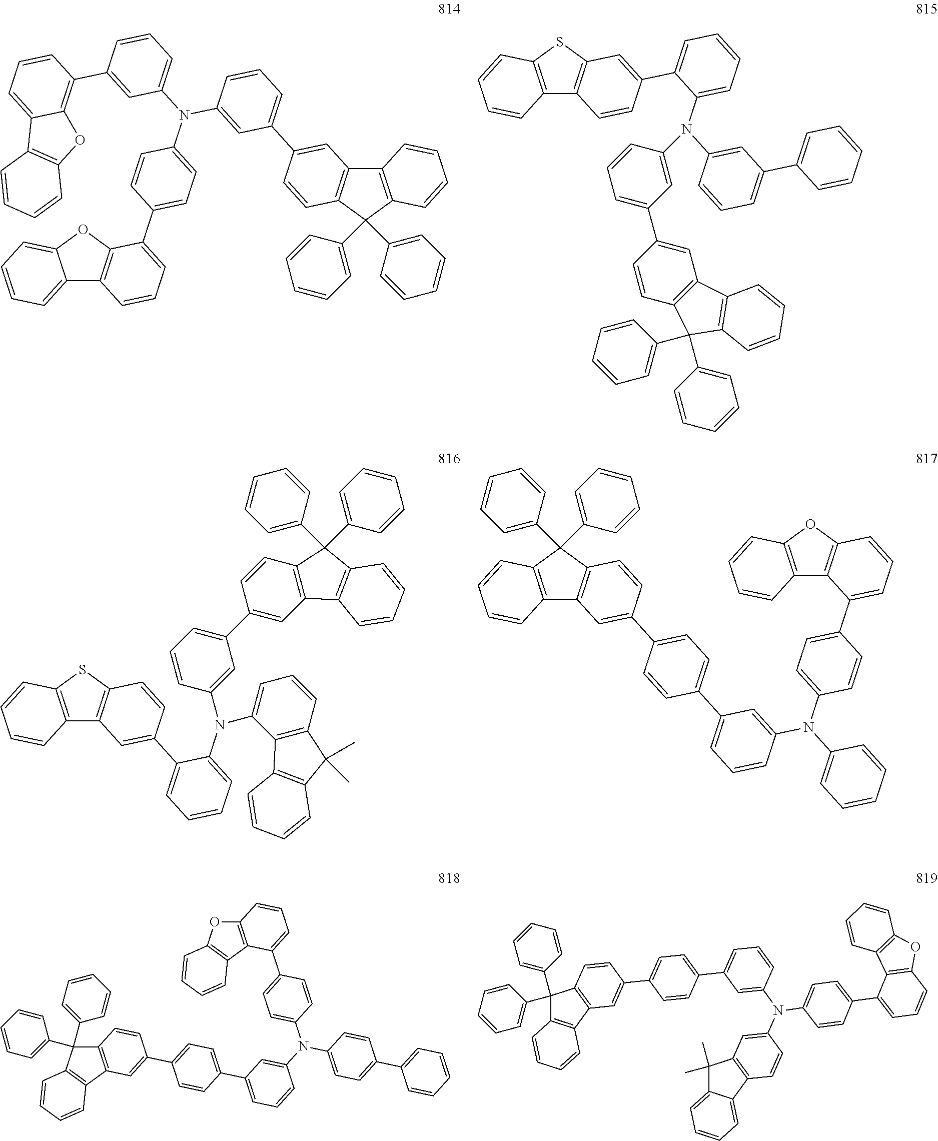

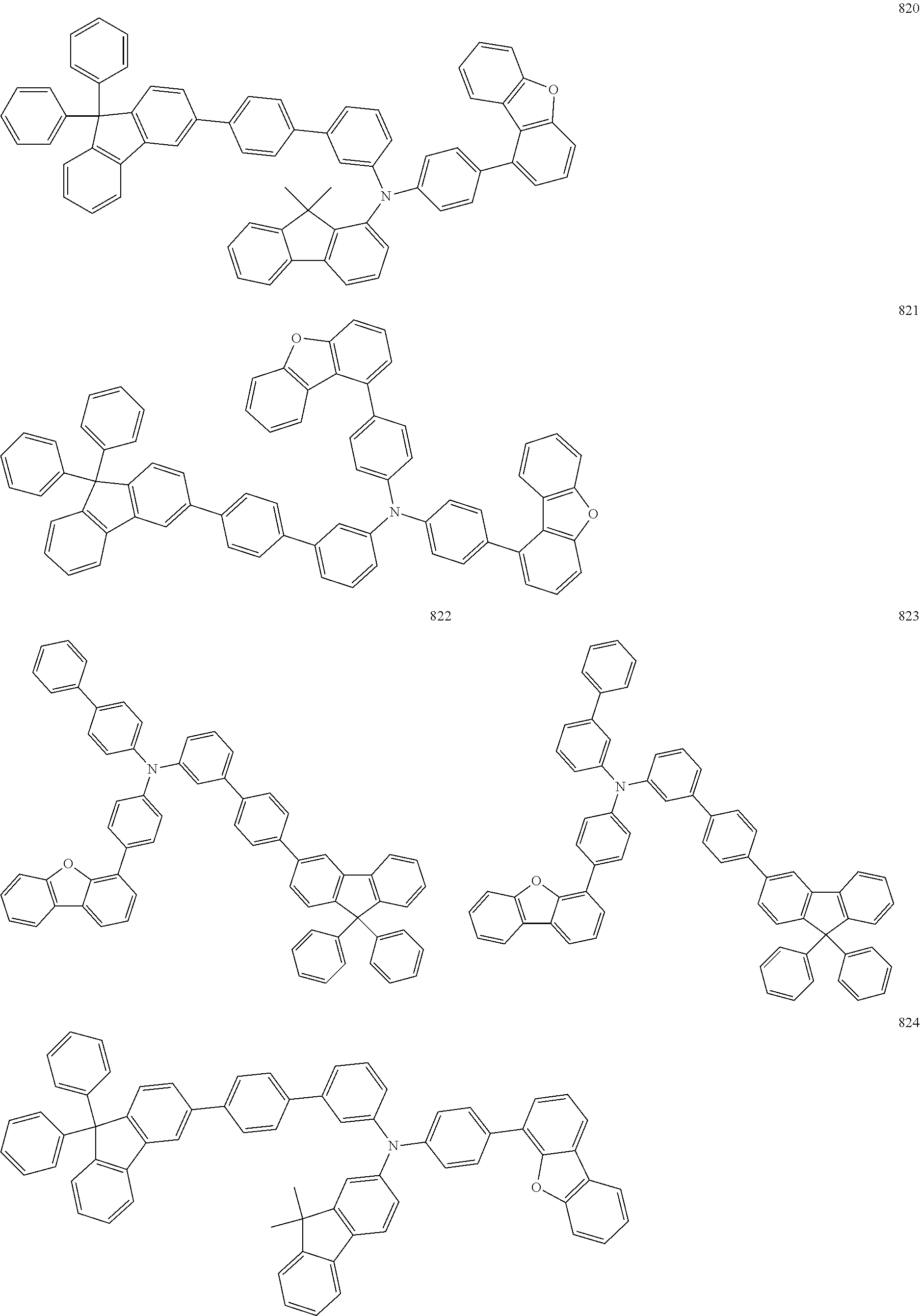

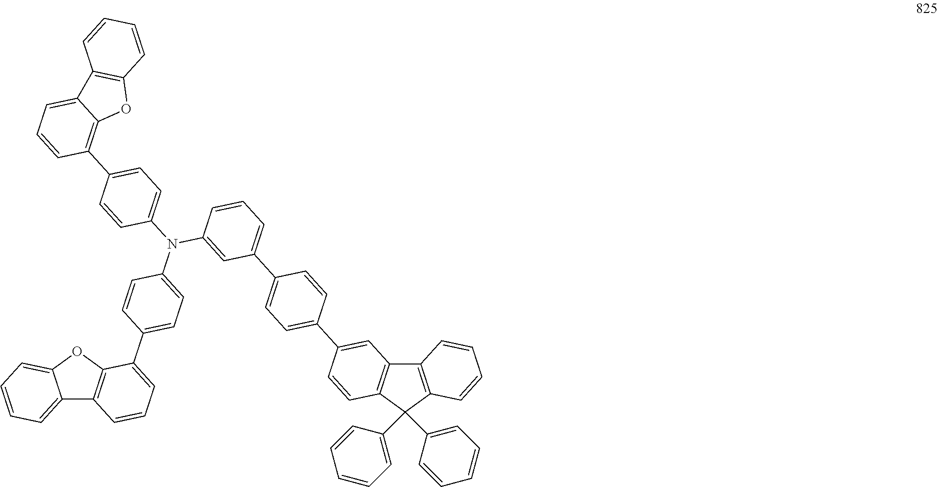 [SMILES: c1ccc(C2(c3ccccc3)c3ccccc3-c3cc(-c4ccc(-c5cccc(N(c6ccc(-c7cccc8c7oc7ccccc78)cc6)c6ccc(-c7cccc8c7oc7ccccc78)cc6)c5)cc4)ccc32)cc1]

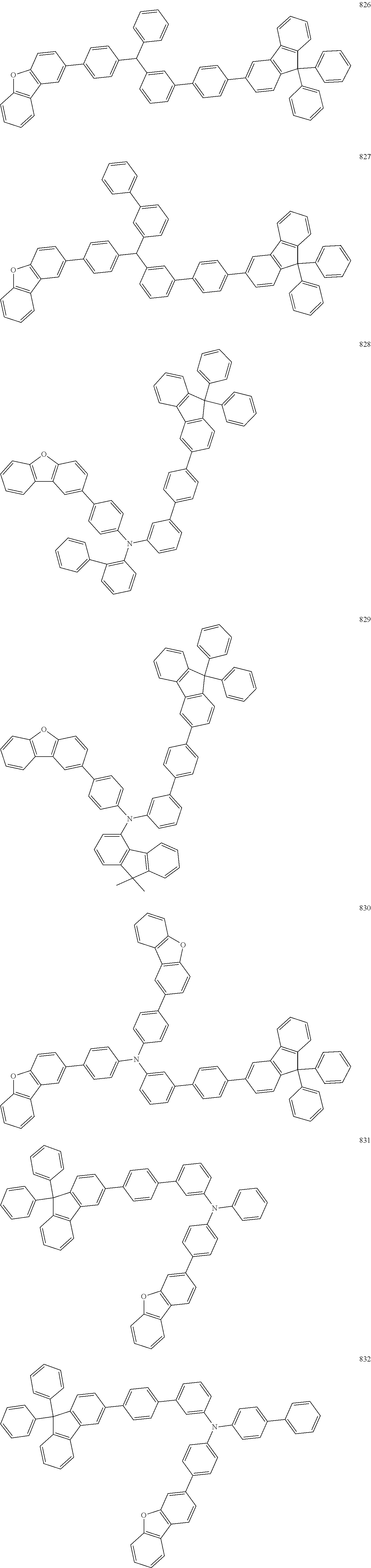

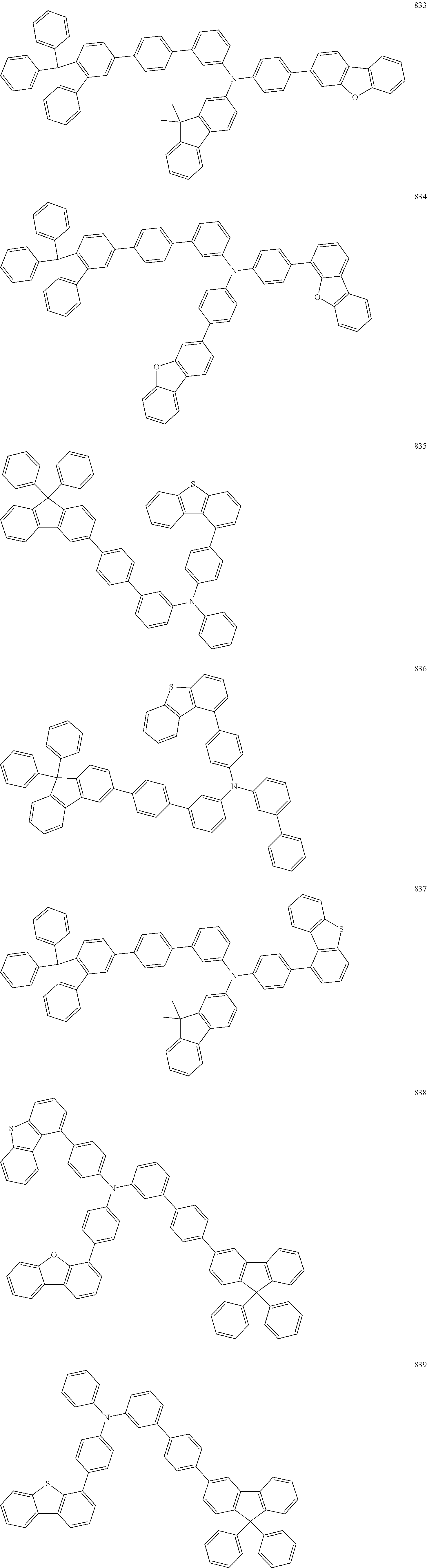

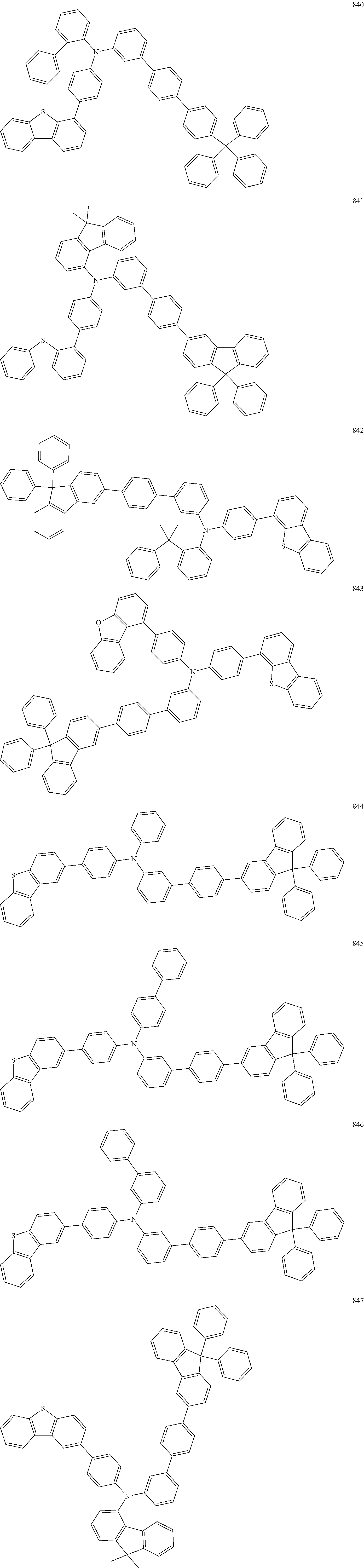

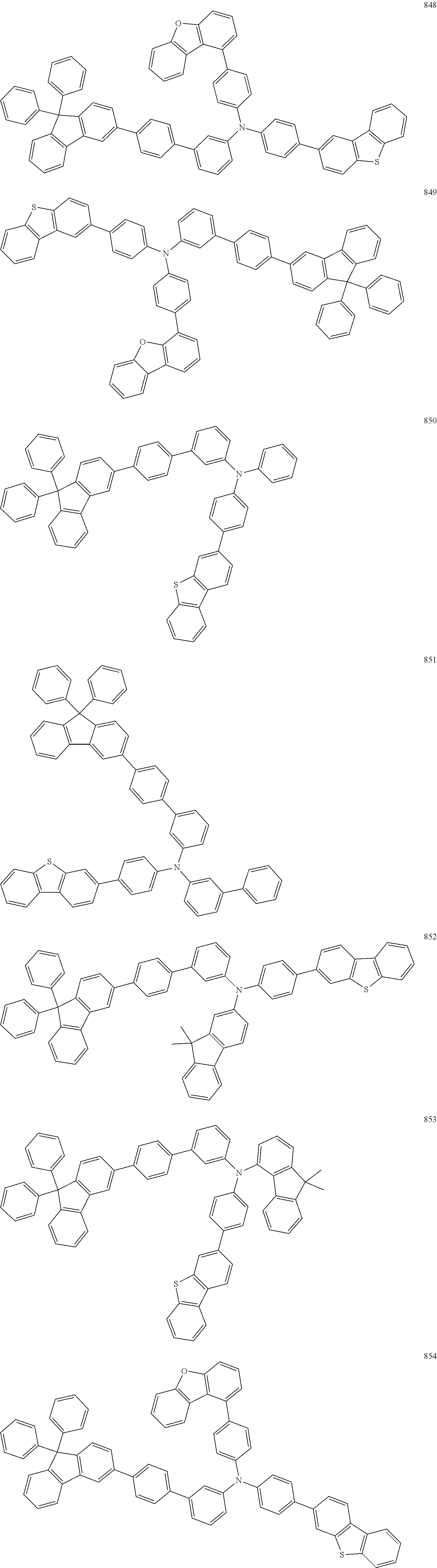

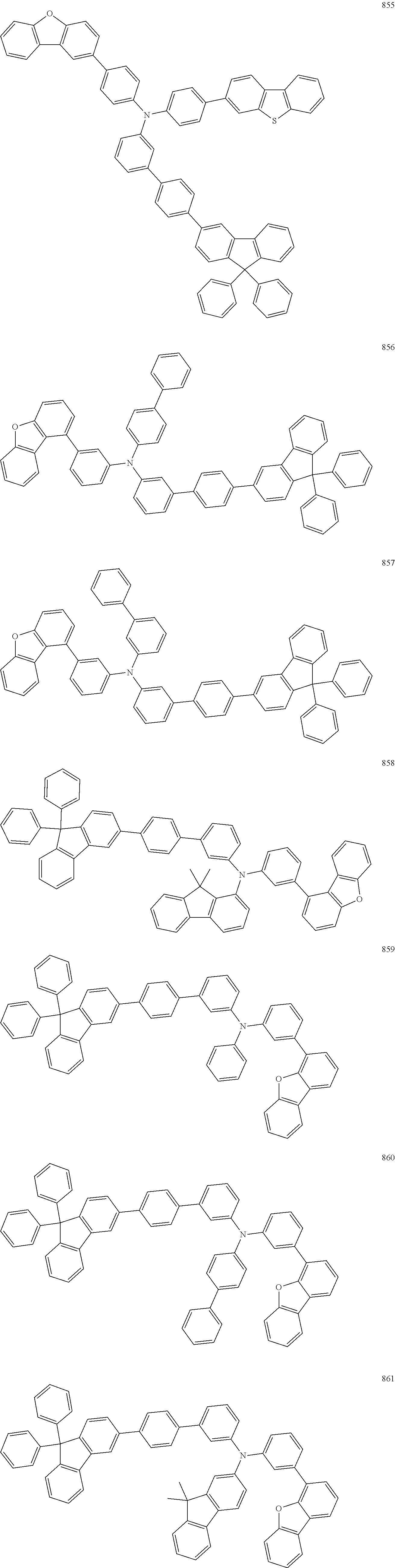

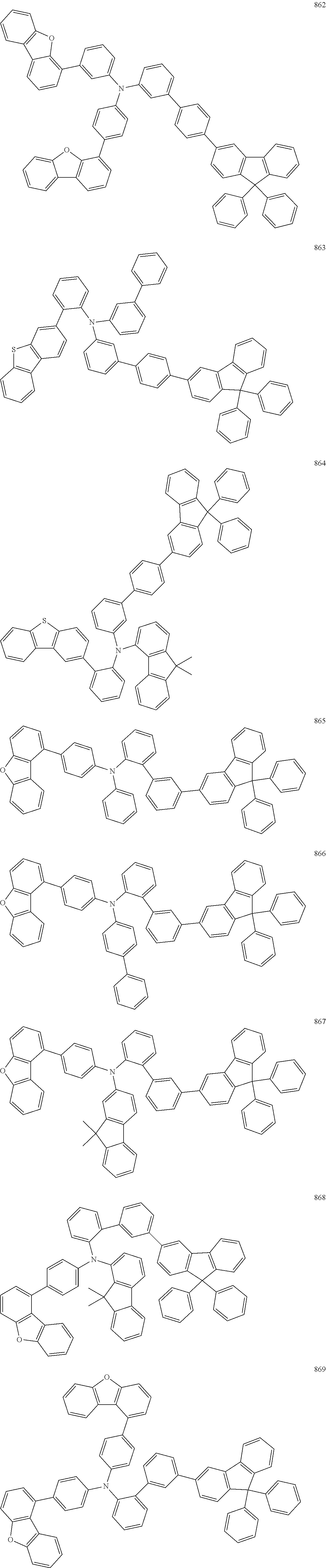

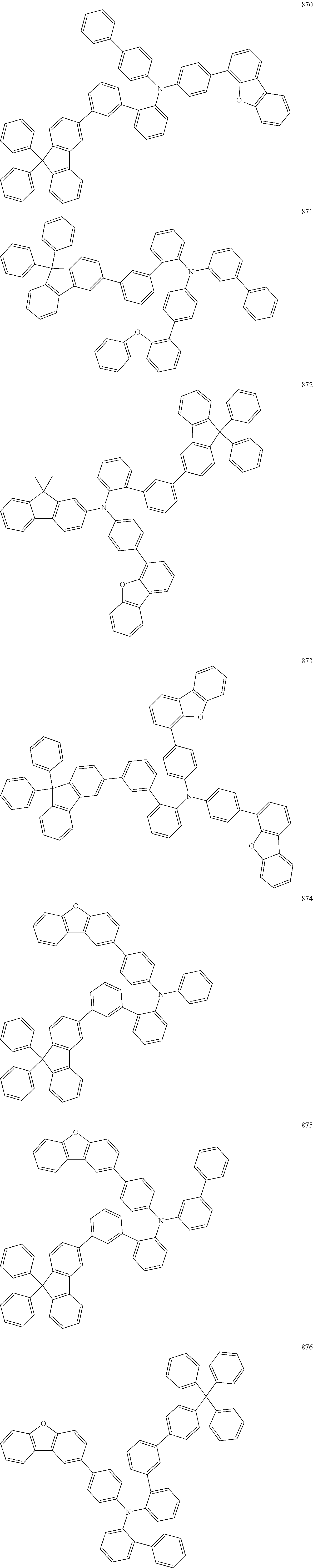

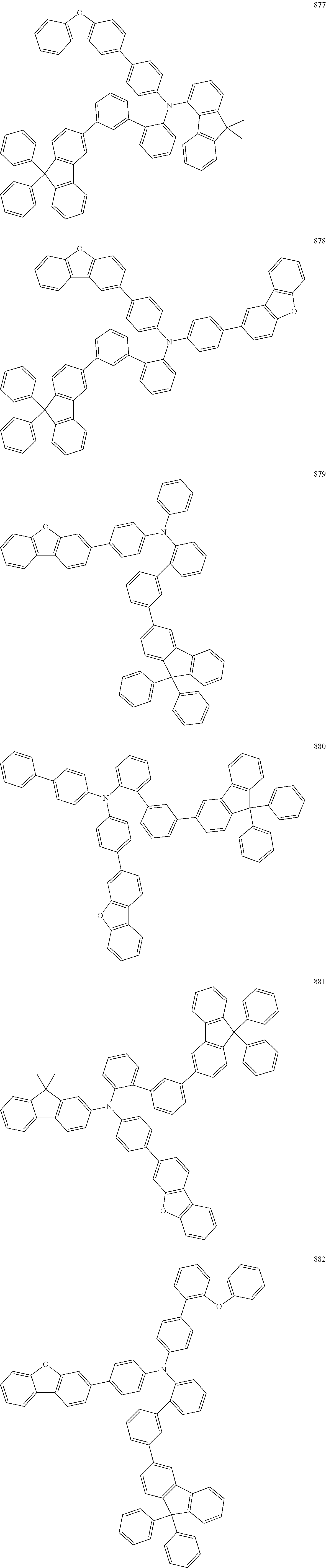

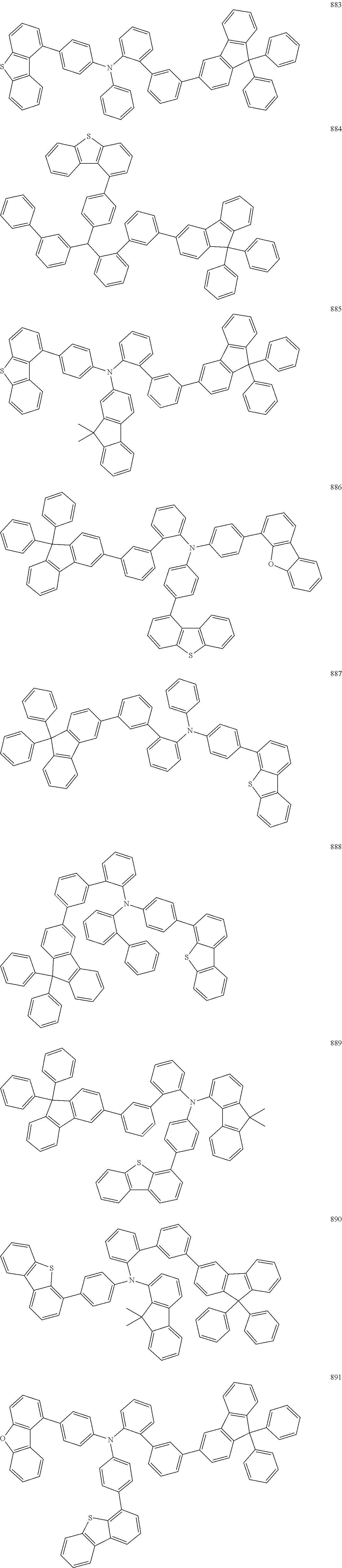

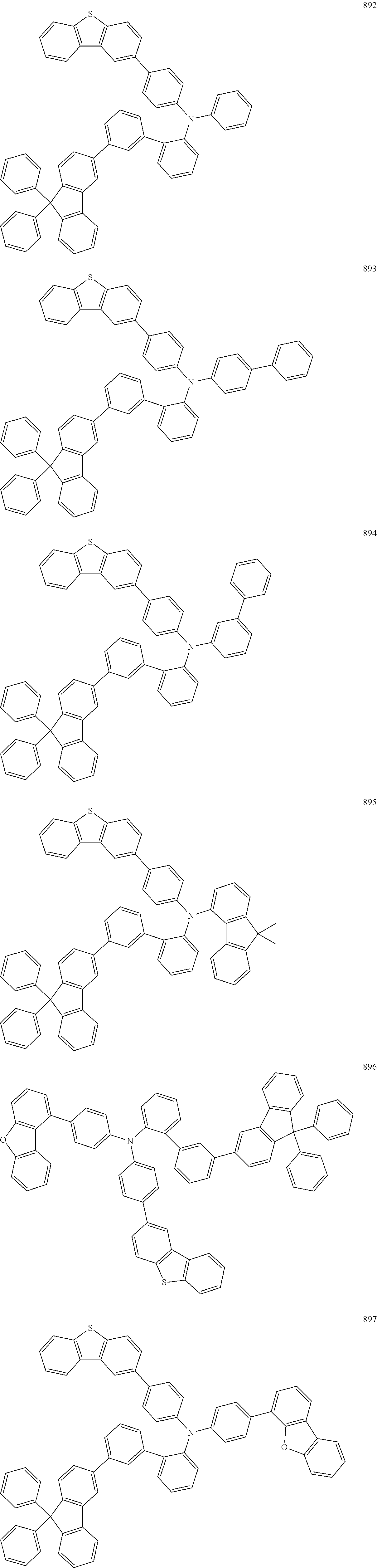

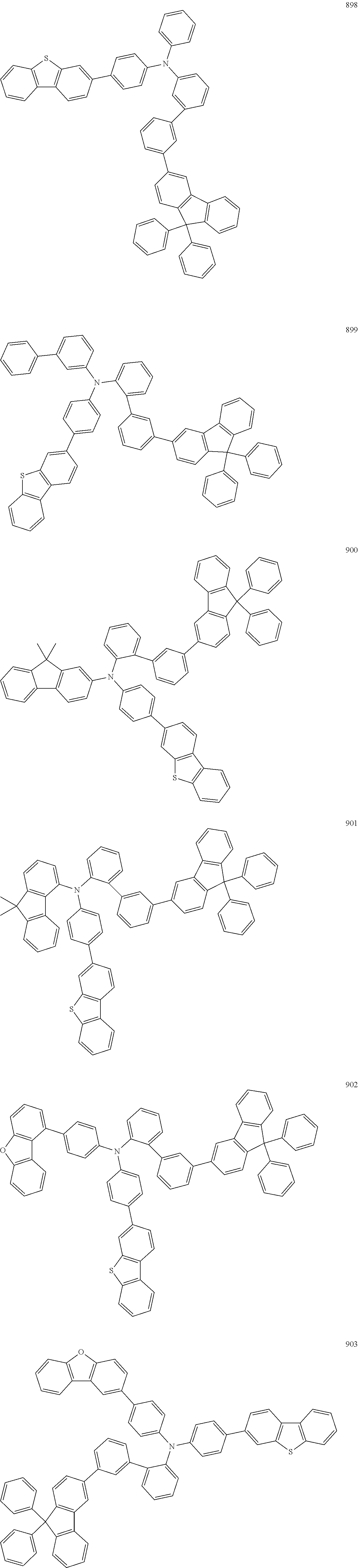

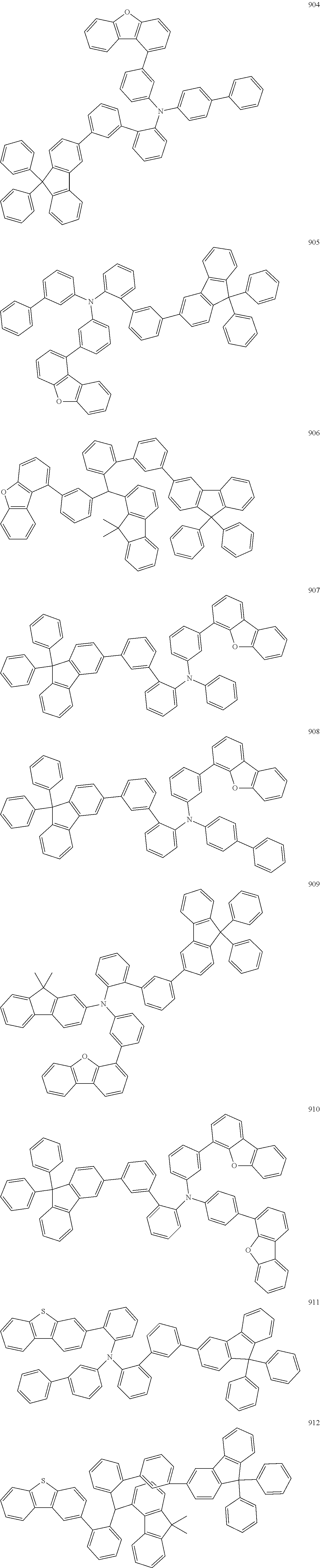

The compounds of the formula (1) can be prepared by means of customary synthesis methods in organic chemistry, for example Buchwald coupling reactions and Suzuki coupling reactions. The compounds of the invention are thus fundamentally synthesized by methods that are very well known to the person skilled in the art in the field. First of all, the fluorene is converted by means of Suzuki coupling. In a second step, the product from the first reaction is converted to the ultimate product by means of a Buchwald reaction.

A preferred synthesis route for the compounds according to the present application is shown below. The person skilled in the art will be able to modify this synthesis route within the scope of his common art knowledge.

The application thus provides a process for preparing a compound of the formula (1) by means of Suzuki and Buchwald coupling.

The invention therefore further provides oligomers, polymers or dendrimers containing one or more compounds of formula (1), wherein the bond(s) to the polymer, oligomer or dendrimer may be localized at any desired positions substituted by R¹, R², R³, R⁴, R⁵, R⁶, R⁷, R⁸, R¹¹ or R¹² in formula (1). According to the linkage of the compound of formula (1), the compound is part of a side chain of the oligomer or polymer or part of the main chain. An oligomer in the context of this invention is understood to mean a compound formed from at least three monomer units. A polymer in the context of the invention is understood to mean a compound formed from at least ten monomer units. The polymers, oligomers or dendrimers of the invention may be conjugated, partly conjugated or nonconjugated. The oligomers or polymers of the invention may be linear, branched or dendritic. In the structures having linear linkage, the units of formula (1) may be joined directly to one another, or they may be joined to one another via a bivalent group, for example via a substituted or unsubstituted alkylene group, via a heteroatom or via a bivalent aromatic or heteroaromatic group. In branched and dendritic structures, it is possible, for example, for three or more units of formula (1) to be joined via a trivalent or higher-valency group, for example via a trivalent or higher-valency aromatic or heteroaromatic group, to give a branched or dendritic oligomer or polymer.

For the repeat units of formula (1) in oligomers, dendrimers and polymers, the same preferences apply as described above for compounds of formula (1).

For preparation of the oligomers or polymers, the monomers of the invention are homopolymerized or copolymerized with further monomers. Suitable and preferred comonomers are selected from fluorenes, spirobifluorenes, paraphenylenes, carbazoles, thiophenes, dihydrophenanthrenes, cis- and trans-indenofluorenes, ketones, phenanthrenes or else two or more of these units. The polymers, oligomers and dendrimers typically contain still further units, for example emitting (fluorescent or phosphorescent) units, for example vinyltriarylamines or phosphorescent metal complexes, and/or charge transport units, especially those based on triarylamines.

The polymers, oligomers and dendrimers of the invention have advantageous properties, especially high lifetimes, high efficiencies and good color coordinates.

The polymers and oligomers of the invention are generally prepared by polymerization of one or more monomer types, of which at least one monomer leads to repeat units of the formula (1) in the polymer. Suitable polymerization reactions are known to those skilled in the art and are described in the literature. Particularly suitable and preferred polymerization reactions which lead to C—C and C—N couplings are as follows:

(A) SUZUKI polymerization;
(B) YAMAMOTO polymerization;
(C) STILLE polymerization; and
(D) HARTWIG-BUCHWALD polymerization.

How the polymerization can be conducted by these methods and how the polymers can then be separated from the reaction medium and purified is known to those skilled in the art and is described in detail in the literature.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, alpha-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, or mixtures of these solvents.

The invention therefore further provides a formulation, especially a solution, dispersion or emulsion, comprising at least one compound of formula (1) or at least one polymer, oligomer or dendrimer containing at least one unit of formula (1) and at least one solvent, preferably an organic solvent. The way in which such solutions can be prepared is known to those skilled in the art.

The compound of formula (1) is suitable for use in an electronic device, especially an organic electroluminescent device (OLED). Depending on the substitution, the compound of the formula (1) can be used in different functions and layers. Preference is given to use as a hole-transporting material in a hole-transporting layer and/or in an electron blocker layer and/or as matrix material in an emitting layer, more preferably in combination with a phosphorescent emitter.

The invention therefore further provides for the use of a compound of formula (1) in an electronic device. This electronic device is preferably selected from the group consisting of organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and more preferably organic light-emitting diodes (OLEDs).

The compounds of the invention are thus particularly suitable for use in organic electroluminescent devices, which also include OLEDs, OLECs, OLETs, QFQDs and O-lasers.

The invention further provides an electronic device comprising at least one compound of formula (1). This electronic device is preferably selected from the abovementioned devices.

Particular preference is given to an organic electroluminescent device, comprising anode, cathode and at least one emitting layer, characterized in that at least one organic layer comprising at least one compound of formula (1) is present in the device. Preference is given to an organic electroluminescent device, comprising anode, cathode and at least one emitting layer, characterized in that at least one organic layer in the device, selected from hole-transporting and emitting layers, comprises at least one compound of formula (1).

A hole-transporting layer is understood here to mean all layers disposed between anode and emitting layer, preferably hole injection layer, hole transport layer and electron blocker layer. A hole injection layer is understood here to mean a layer that directly adjoins the anode. A hole transport layer is understood here to mean a layer which is between the anode and emitting layer but does not directly adjoin the anode, and preferably does not directly adjoin the emitting layer either. An electron blocker layer is understood here to mean a layer which is between the anode and emitting layer and directly adjoins the emitting layer. An electron blocker layer preferably has a high-energy LUMO and hence prevents electrons from exiting from the emitting layer.

Apart from the cathode, anode and emitting layer, the electronic device may comprise further layers. These are selected, for example, from in each case one or more hole injection layers, hole transport layers, hole blocker layers, electron transport layers, electron injection layers, electron blocker layers, exciton blocker layers, interlayers, charge generation layers and/or organic or inorganic p/n junctions. However, it should be pointed out that not every one of these layers need necessarily be present and the choice of layers always depends on the compounds used and especially also on whether the device is a fluorescent or phosphorescent electroluminescent device. The sequence of layers in the electronic device is preferably as follows:

-anode-
-hole injection layer-
-hole transport layer-
-optionally further hole transport layers-
-emitting layer-
-optionally hole blocker layer-
-electron transport layer-
-electron injection layer-
-cathode-.

At the same time, it should be pointed out again that not all the layers mentioned need be present and/or that further layers may additionally be present.

The organic electroluminescent device of the invention may contain two or more emitting layers. More preferably, these emission layers have several emission maxima between 380 nm and 750 nm overall, such that the overall result is white emission; in other words, various emitting compounds which may fluoresce or phosphoresce and which emit blue, green, yellow, orange or red light are used in the emitting layers. Especially preferred are three-layer systems, i.e. systems having three emitting layers, wherein one of the three layers in each case shows blue emission, one of the three layers in each case shows green emission, and one of the three layers in each case shows orange or red emission. The compounds of the invention here are preferably present in a hole-transporting layer or in the emitting layer. It should be noted that, for the production of white light, rather than a plurality of colour-emitting emitter compounds, an emitter compound used individually which emits over a broad wavelength range may also be suitable.

It is preferable that the compound of the formula (1) is used as hole transport material. The emitting layer here may be a fluorescent emitting layer, or it may be a phosphorescent emitting layer. The emitting layer is preferably a blue-fluorescing layer or a green-phosphorescing layer. Particular preference is given to the use of the compounds of the invention as hole-conducting electron blocker material in an electron blocker layer.

When the device containing the compound of the formula (1) contains a phosphorescent emitting layer, it is preferable that this layer contains two or more, preferably exactly two, different matrix materials (mixed matrix system). Preferred embodiments of mixed matrix systems are described in detail further down.

If the compound of formula (1) is used as hole transport material in a hole transport layer, a hole injection layer or an electron blocker layer, the compound can be used as pure material, i.e. in a proportion of 100%, in the hole transport layer, or it can be used in combination with one or more further compounds.

In a preferred embodiment, a hole-transporting layer or an electron blocker layer comprising the compound of the formula (1) additionally comprises one or more further hole-transporting compounds. These further hole-transporting compounds are preferably selected from triarylamine compounds, more preferably from monotriarylamine compounds. They are most preferably selected from the preferred embodiments of hole transport materials that are specified further down. In the preferred embodiment described, the compound of the formula (1) and the one or more further hole-transporting compounds are preferably each present in a proportion of at least 10%, more preferably each in a proportion of at least 20%.

In a preferred embodiment, a hole-transporting layer or an electron blocker layer comprising the compound of the formula (1) additionally comprises one or more p-dopants. p-Dopants used according to the present invention are preferably those organic electron acceptor compounds capable of oxidizing one or more of the other compounds in the mixture.

Particularly preferred as p-dopants are quinodimethane compounds, azaindenofluorenediones, azaphenalenes, azatriphenylenes, I₂, metal halides, preferably transition metal halides, metal oxides, preferably metal oxides comprising at least one transition metal or a metal from main group 3, and transition metal complexes, preferably complexes of Cu, Co, Ni, Pd and Pt with ligands containing at least one oxygen atom as binding site. Preference is further given to transition metal oxides as dopants, preferably oxides of rhenium, molybdenum and tungsten, more preferably Re₂O₇, MoO₃, WO₃ and ReO₃. Still further preference is given to complexes of bismuth in the (III) oxidation state, more particularly bismuth(II) complexes with electron-deficient ligands, more particularly carboxylate ligands.

The p-dopants are preferably in substantially homogeneous distribution in the p-doped layers. This can be achieved, for example, by co-evaporation of the p-dopant and the hole transport material matrix. The p-dopant is preferably present in a proportion of 1% to 10% in the p-doped layer.

Preferred p-dopants are especially the following compounds:

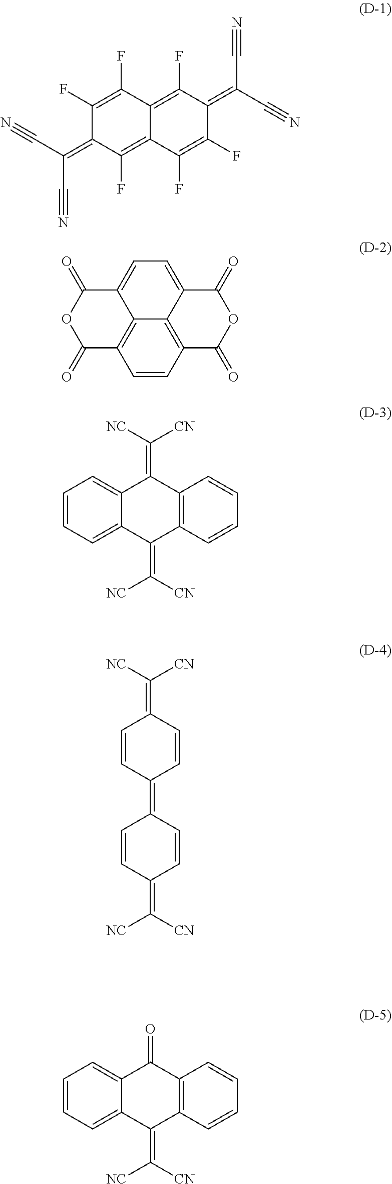

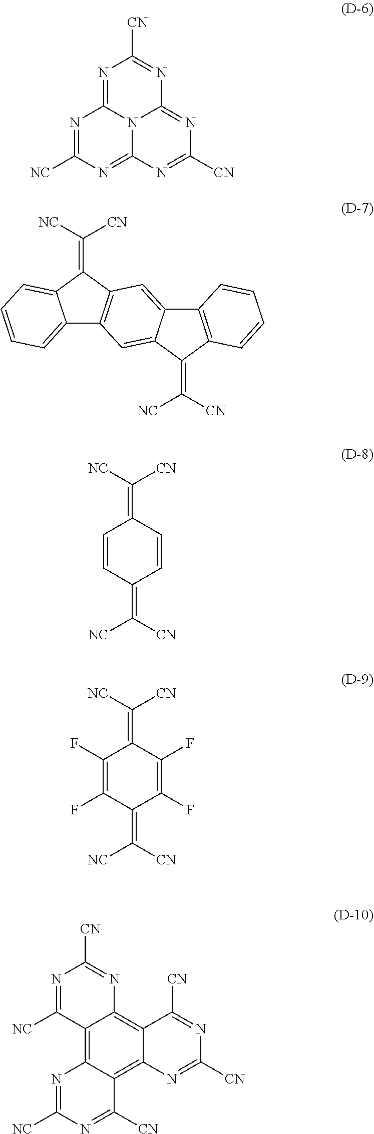 [SMILES: N#CC1=NC2=NC(C#N)=NC3=NC(C#N)=NC(=N1)N23 ; N#CC(C#N)=C1c2ccccc2-c2cc3c(cc21)-c1ccccc1C3=C(C#N)C#N ; N#CC(C#N)=c1ccc(=C(C#N)C#N)cc1 ; N#CC(C#N)=c1c(F)c(F)c(=C(C#N)C#N)c(F)c1F ; N#Cc1nc(C#N)c2c(n1)c1c(C#N)nc(C#N)nc1c1c(C#N)nc(C#N)nc21]

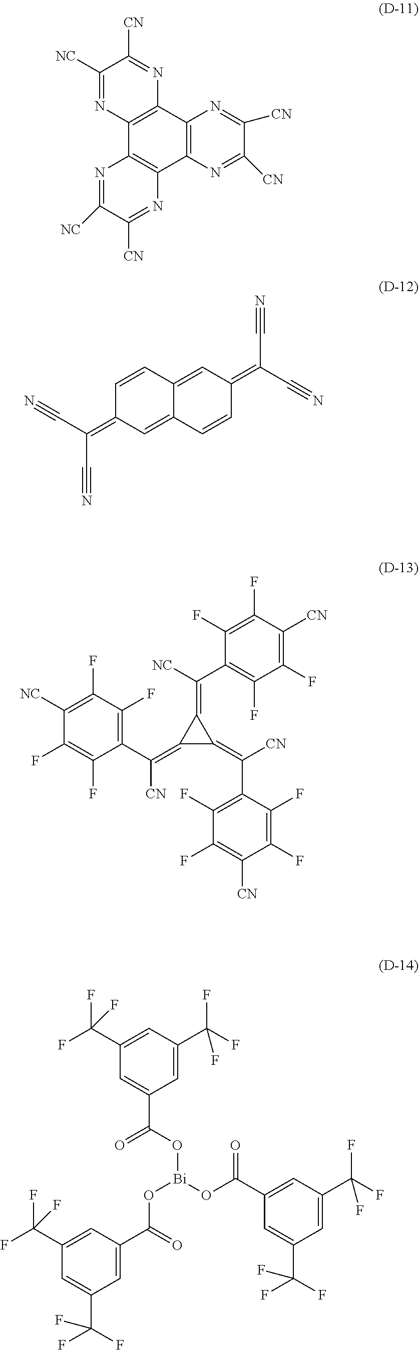

In a preferred embodiment, a hole injection layer that conforms to one of the following embodiments is present in the device: a) it contains a triarylamine and a p-dopant; or b) it contains a single electron-deficient material (electron acceptor). In a preferred embodiment of embodiment a), the triarylamine is a monotriarylamine, especially one of the preferred triarylamine derivatives mentioned further down. In a preferred embodiment of embodiment b), the electron-deficient material is a hexaazatriphenylene derivative as described in US 2007/0092755.

The compound of the formula (1) may be present in a hole injection layer, in a hole transport layer and/or in an electron blocker layer of the device. When the compound is present in a hole injection layer or in a hole transport layer, it has preferably been p-doped, meaning that it is in mixed form with a p-dopant, as described above, in the layer.

More preferably, the compound of the formula (1) is present in an electron blocker layer. In this case, it is preferably not p-doped. Further preferably, in this case, it is preferably in the form of a single compound in the layer without addition of a further compound.

Preference is given to using the compound of the formula (1) in the electron blocker layer of the device, where the device has green emission. The device in that case preferably contains at least one fluorescent or phosphorescent emitter that emits green light, preference being given to green-emitting phosphorescent emitters.

In an alternative preferred embodiment, the compound of the formula (1) is used in an emitting layer as matrix material in combination with one or more emitting compounds, preferably phosphorescent emitting compounds. The phosphorescent emitting compounds here are preferably selected from red-phosphorescing and green-phosphorescing compounds.

The proportion of the matrix material in the emitting layer in this case is between 50.0% and 99.9% by volume, preferably between 80.0% and 99.5% by volume, and more preferably between 85.0% and 97.0% by volume.

Correspondingly, the proportion of the emitting compound is between 0.1% and 50.0% by volume, preferably between 0.5% and 20.0% by volume, and more preferably between 3.0% and 15.0% by volume.

An emitting layer of an organic electroluminescent device may also contain systems comprising a plurality of matrix materials (mixed matrix systems) and/or a plurality of emitting compounds. In this case too, the emitting compounds are generally those compounds having the smaller proportion in the system and the matrix materials are those compounds having the greater proportion in the system. In individual cases, however, the proportion of a single matrix material in the system may be less than the proportion of a single emitting compound.

It is preferable that the compounds of formula (1) are used as a component of mixed matrix systems, preferably for phosphorescent emitters. The mixed matrix systems preferably comprise two or three different matrix materials, more preferably two different matrix materials. Preferably, in this case, one of the two materials is a material having hole-transporting properties and the other material is a material having electron-transporting properties. It is further preferable when one of the materials is selected from compounds having a large energy differential between HOMO and LUMO (wide-bandgap materials). The compound of the formula (1) in a mixed matrix system is preferably the matrix material having hole-transporting properties. Correspondingly, when the compound of the formula (1) is used as matrix material for a phosphorescent emitter in the emitting layer of an OLED, a second matrix compound having electron-transporting properties is present in the emitting layer. The two different matrix materials may be present here in a ratio of 1:50 to 1:1, preferably 1:20 to 1:1, more preferably 1:10 to 1:1 and most preferably 1:4 to 1:1.

The desired electron-transporting and hole-transporting properties of the mixed matrix components may, however, also be combined mainly or entirely in a single mixed matrix component, in which case the further mixed matrix component(s) fulfil(s) other functions.

The inventive compounds of the formula (1) may also be used together with other materials in the form of a solid-state mixture, in order to be vapour-deposited from a source for production of a layer of an organic electronic device. The additional component is preferably a hole transport material, an electron blocker material or a matrix material. Such solid-state mixtures are also referred to as premixed systems.

The present application therefore also relates to solid-state mixtures comprising a compound of the formula (1) and at least one further hole transport material, an electron blocker material or a matrix material.

The present application also relates to a process for producing a layer of an organic electronic device by evaporation of a solid-state mixture comprising a compound of the formula (1) and at least one further hole transport material, an electron blocker material or a matrix material.

The present application further provides mixtures or compositions comprising one or more compounds of the formula (1) and at least one further material selected from the group consisting of the hole transport materials, hole injection materials, p-dopants, electron blocker materials, matrix materials, emitters and electron transport materials. The materials used are well known to the person skilled in the art and may in principle all be used for this purpose. Especially suitable for this purpose are the materials that have been mentioned elsewhere in the present application. The emitter may be a fluorescent or phosphorescent emitter, preference being given to phosphorescent emitters. The matrix materials typically include the hole-conducting and electron-conducting matrix materials, but also the bipolar matrix materials and what are called the wide-bandgap materials, i.e. those materials that are used as matrix material and have a wide bandgap (i.e. HOMO-LUMO separation), which is preferably not less than 3.0 eV.

Preference is given to using the following material classes in the abovementioned layers of the device:

Phosphorescent Emitters:

The term “phosphorescent emitters” typically encompasses compounds where the emission of light is effected through a spin-forbidden transition, for example a transition from an excited triplet state or a state having a higher spin quantum number, for example a quintet state.

Suitable phosphorescent emitters are especially compounds which, when suitably excited, emit light, preferably in the visible region, and also contain at least one atom of atomic number greater than 20, preferably greater than 38, and less than 84, more preferably greater than 56 and less than 80. Preference is given to using, as phosphorescent emitters, compounds containing copper, molybdenum, tungsten, rhenium, ruthenium, osmium, rhodium, iridium, palladium, platinum, silver, gold or europium, especially compounds containing iridium, platinum or copper.

In the context of the present invention, all luminescent iridium, platinum or copper complexes are considered to be phosphorescent compounds.

In general, all phosphorescent complexes as used for phosphorescent OLEDs according to the prior art and as known to those skilled in the art in the field of organic electroluminescent devices are suitable for use in the devices of the invention. Further examples of suitable phosphorescent emitters are shown in the following table:

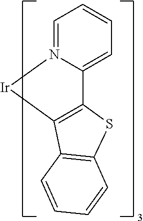

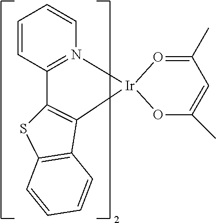

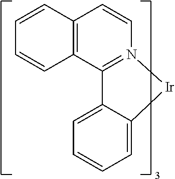

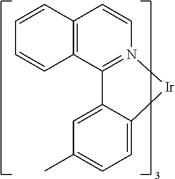

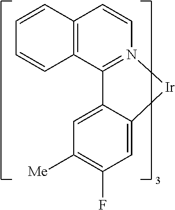

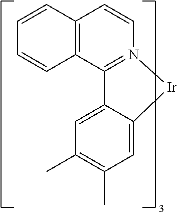

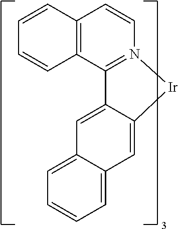

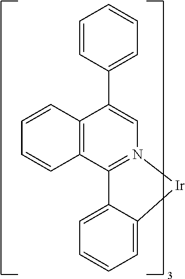

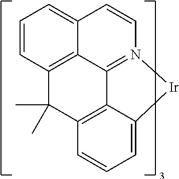

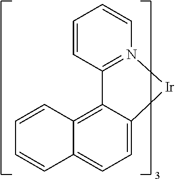

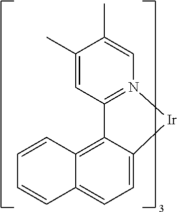

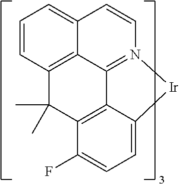

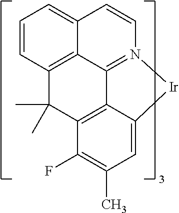

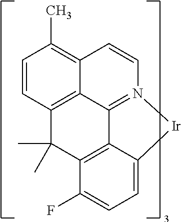

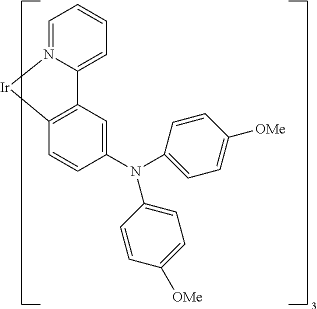

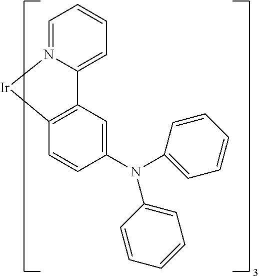

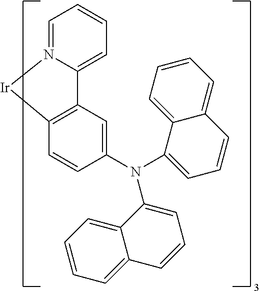

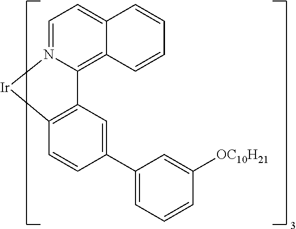

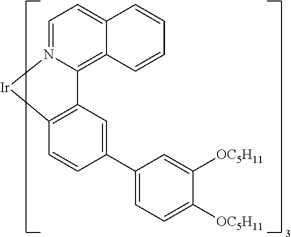

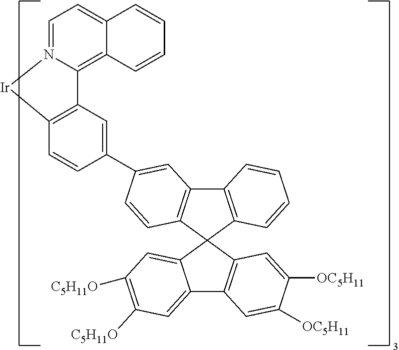

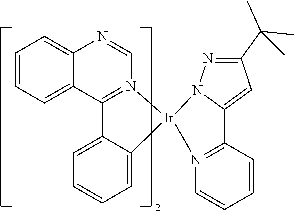

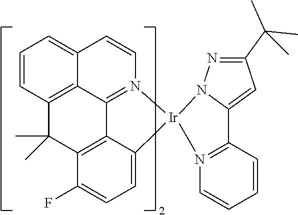

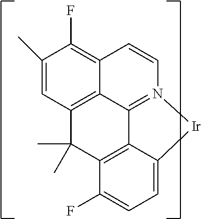

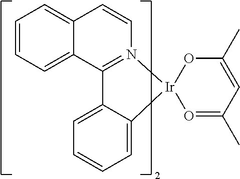

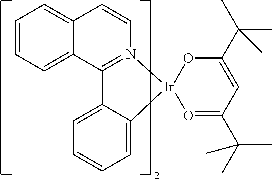

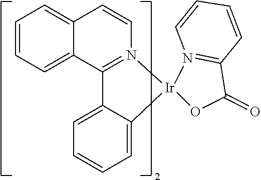

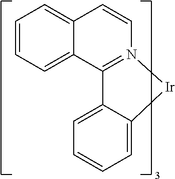

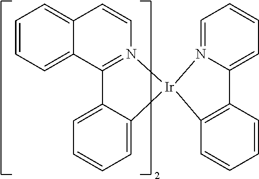

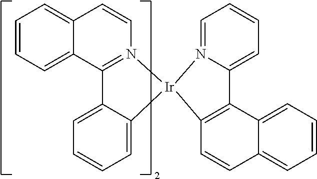

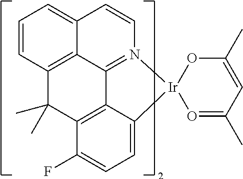

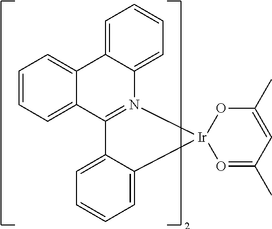

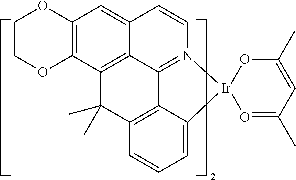

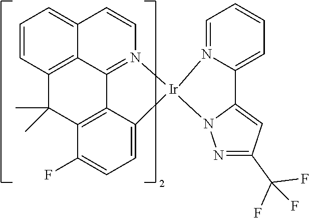

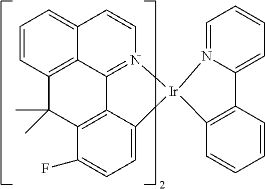

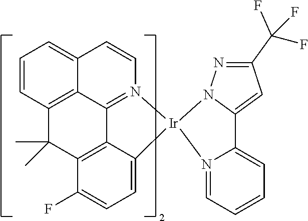

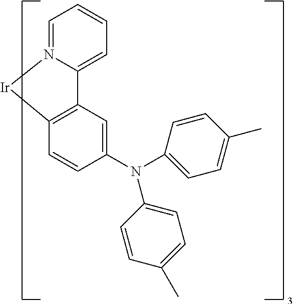

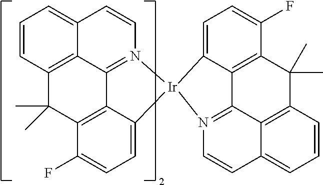

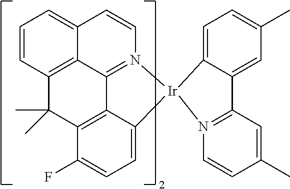

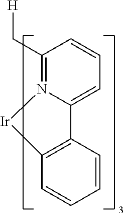

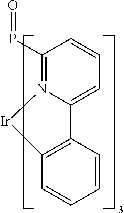

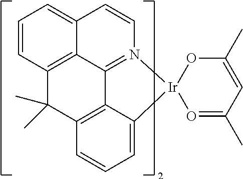

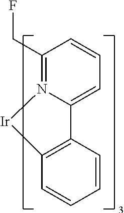

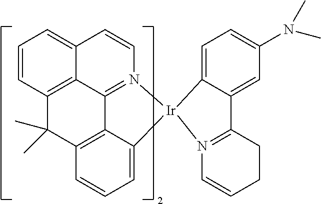

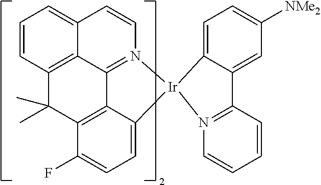

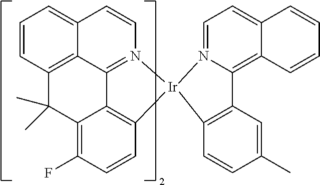

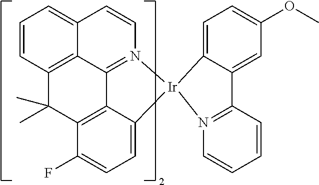

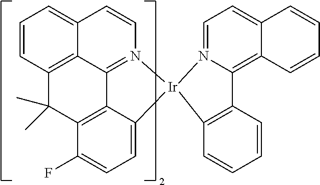

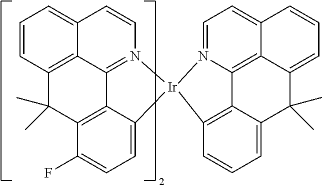

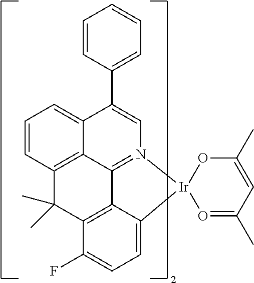

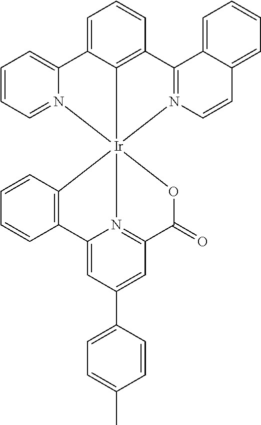

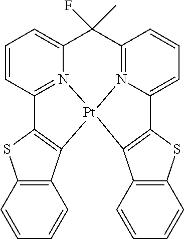

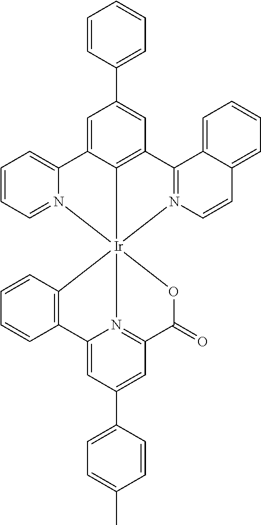

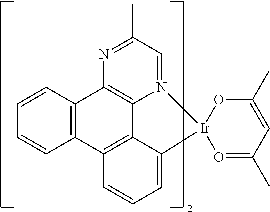

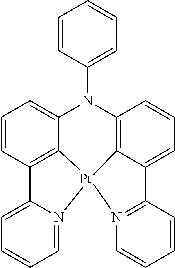

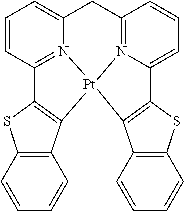

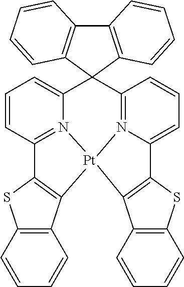

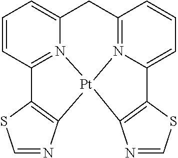

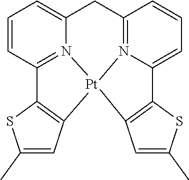

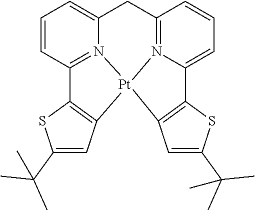

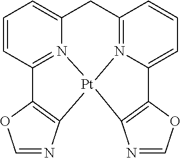

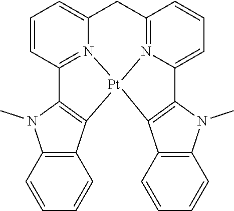

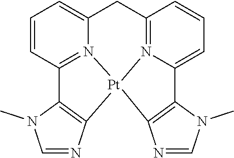

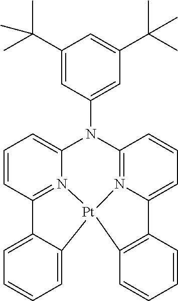

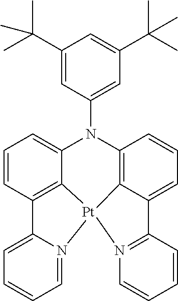

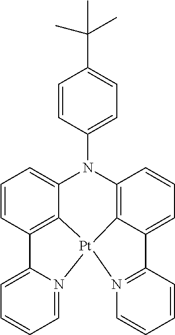

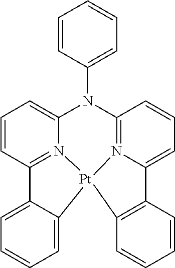

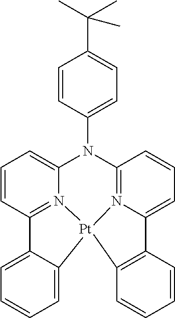

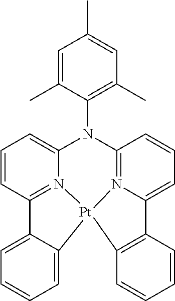

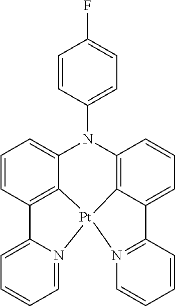

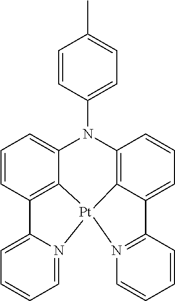

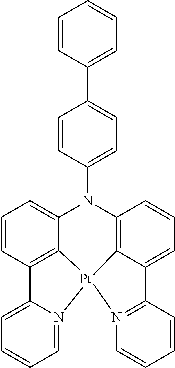

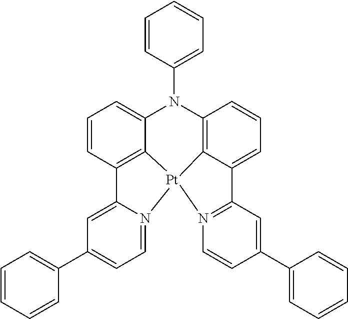

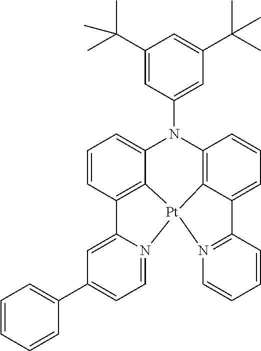

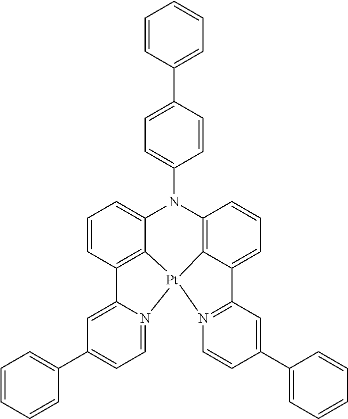

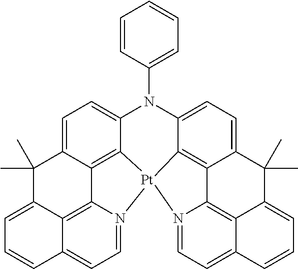

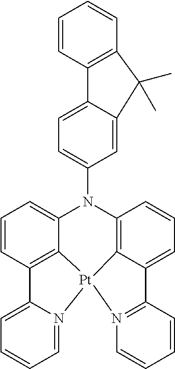

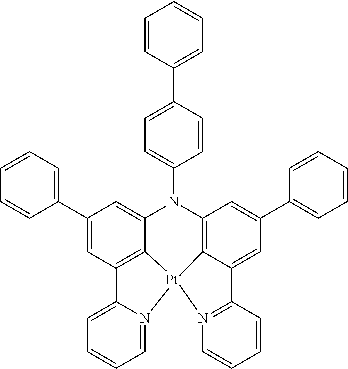

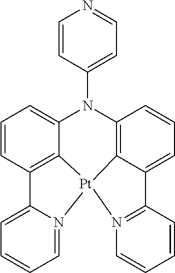

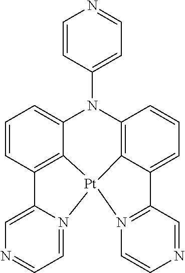

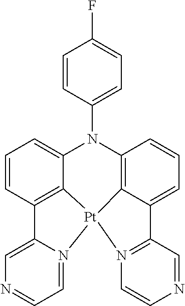

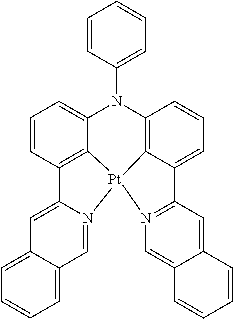

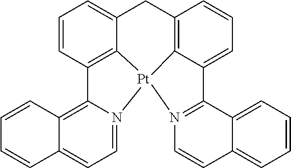

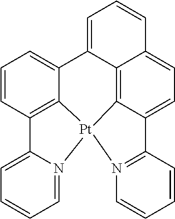

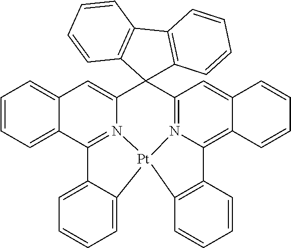

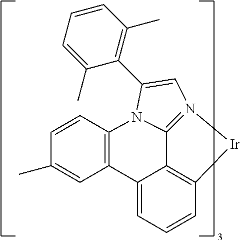

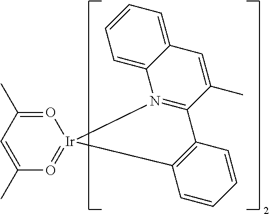

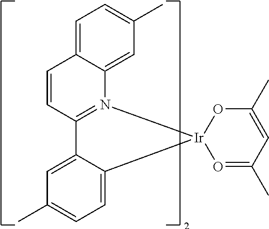

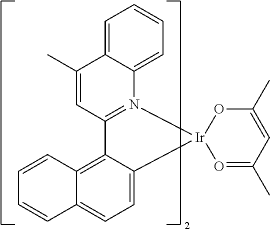

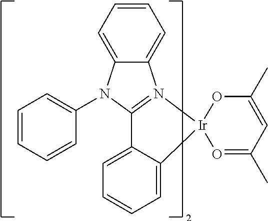

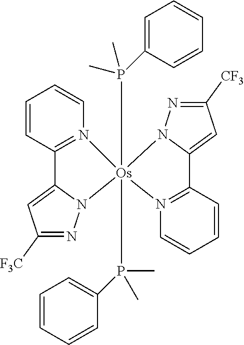

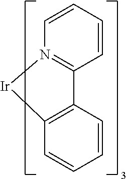

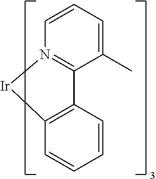

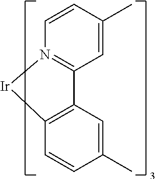

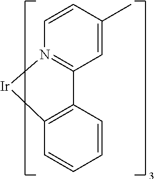

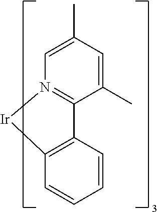

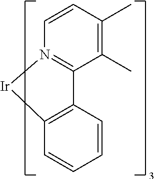

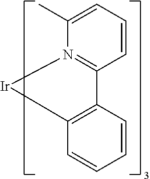

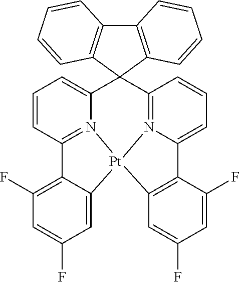

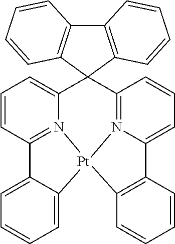

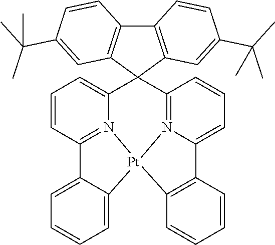

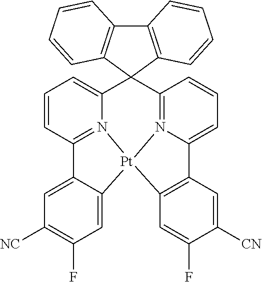

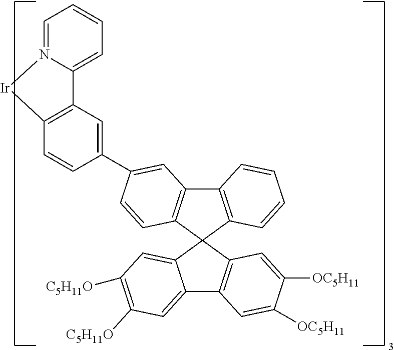

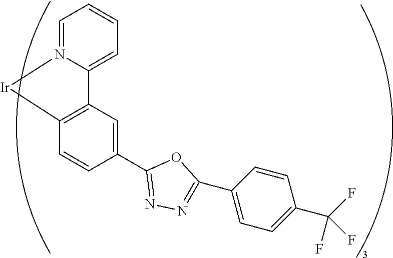

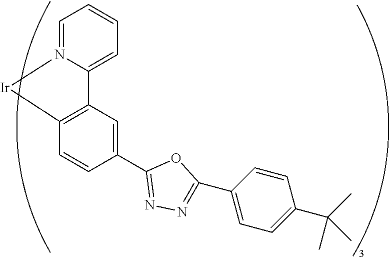

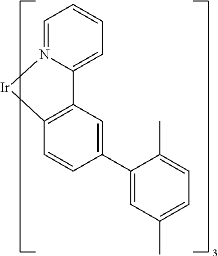

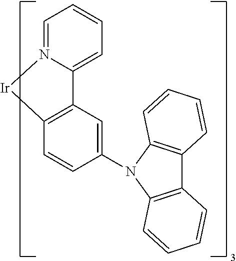

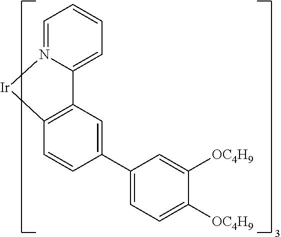

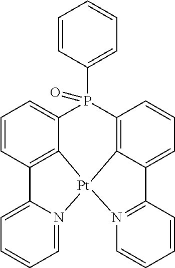

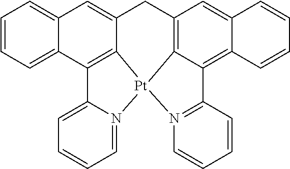

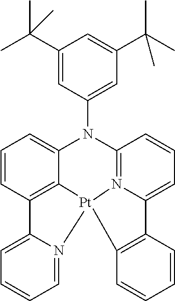

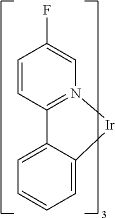

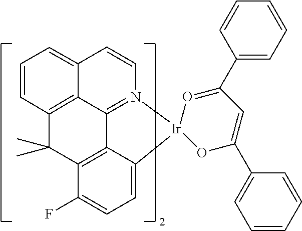

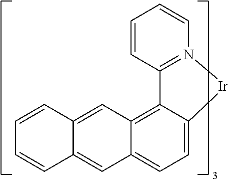

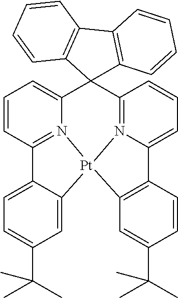

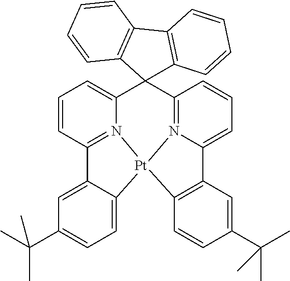

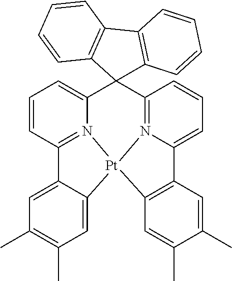

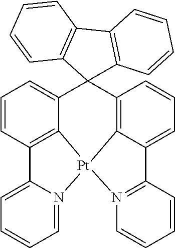

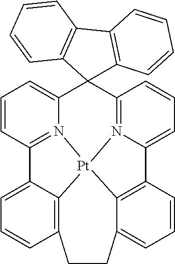

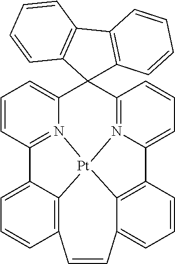

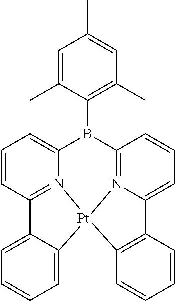

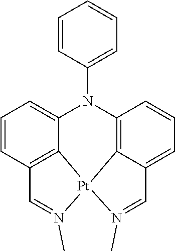

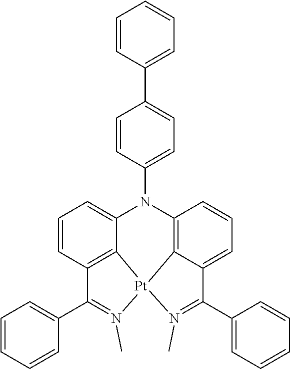

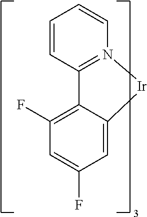

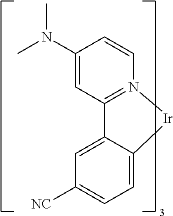

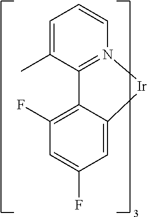

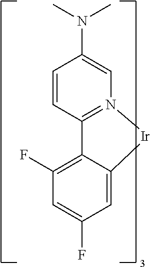

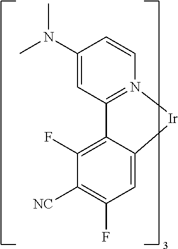

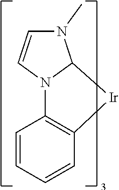

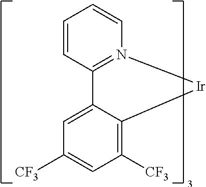

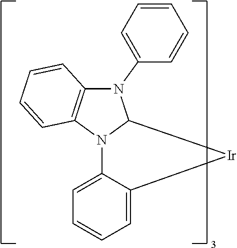

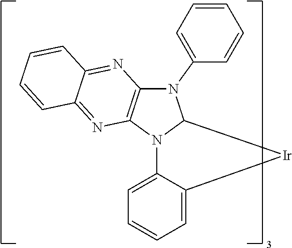

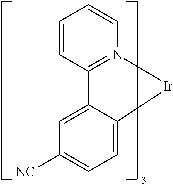

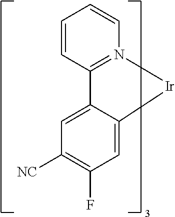

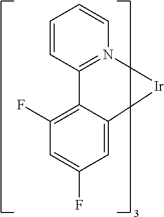

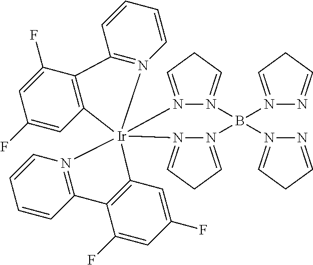

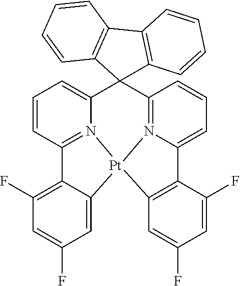

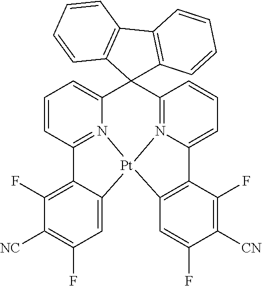

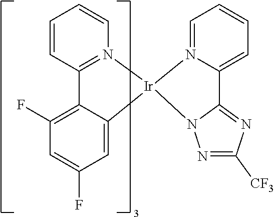

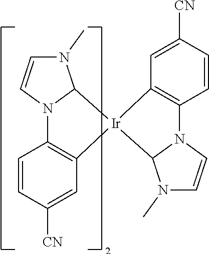

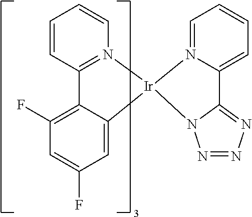

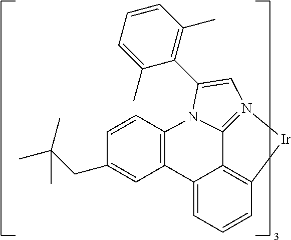

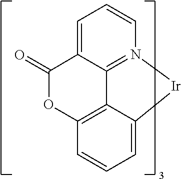

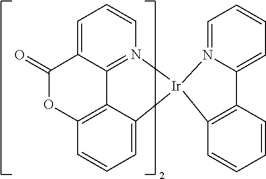

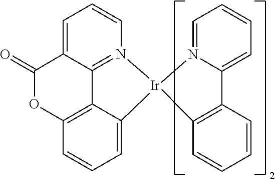

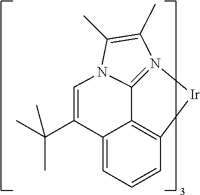

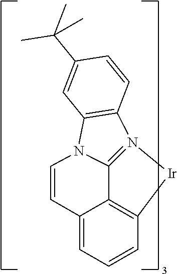

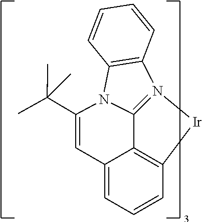

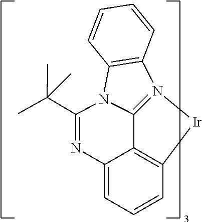

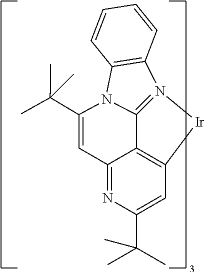

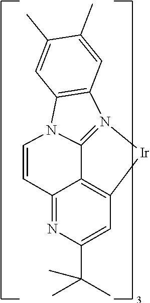

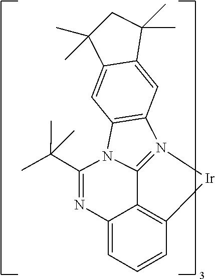

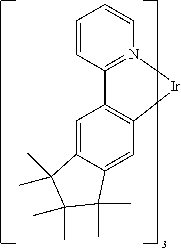

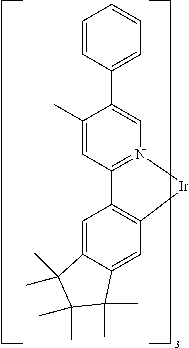

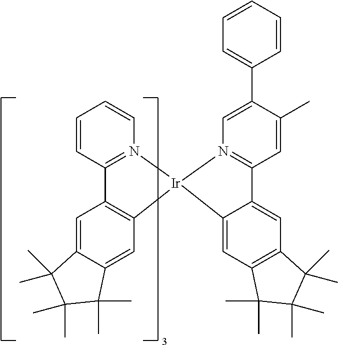

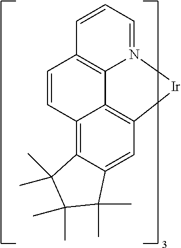

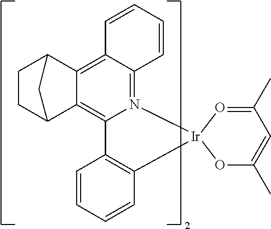

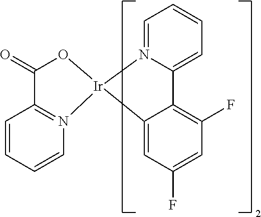

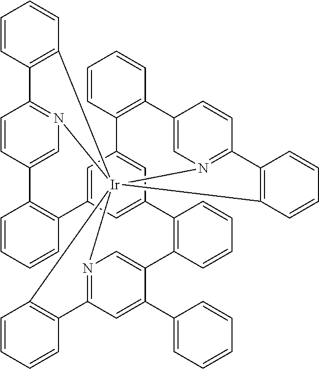

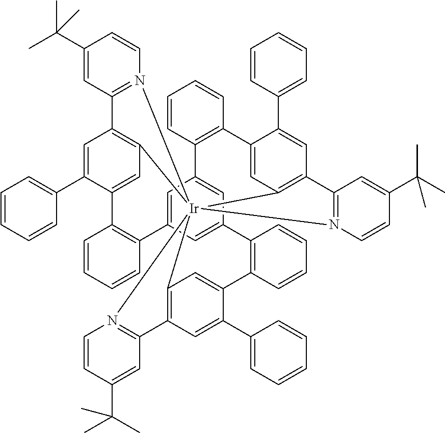

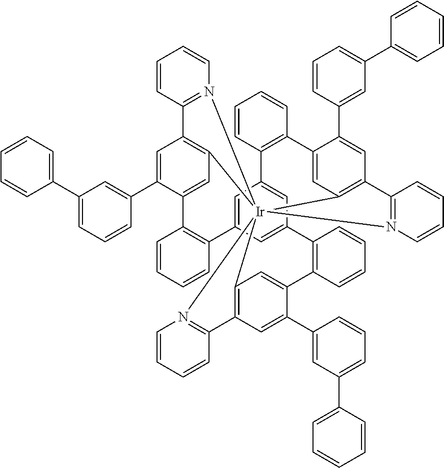

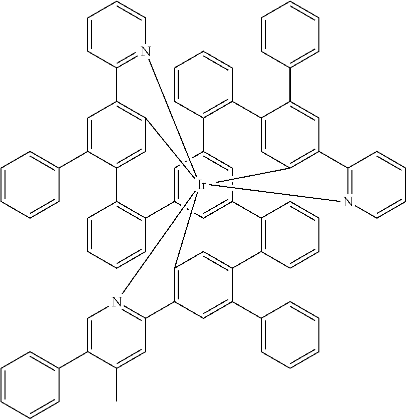

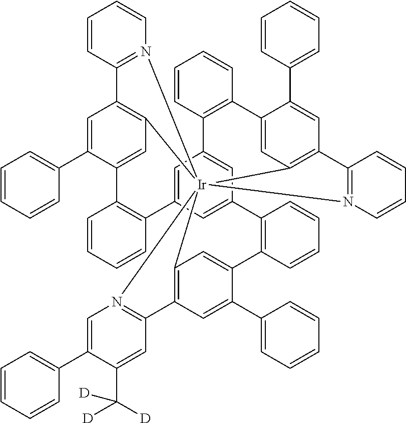

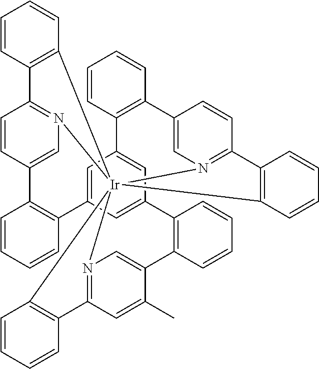

Fluorescent Emitters:

Preferred fluorescent emitting compounds are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. An aromatic anthraceneamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9,10 position. Aromatic pyreneamines, pyrenediamines, chryseneamines and chrysenediamines are defined analogously, where the diarylamino groups are bonded to the pyrene preferably in the 1 position or 1,6 position. Further preferred emitting compounds are indenofluoreneamines or -diamines, benzoindenofluoreneamines or -diamines, and dibenzoindenofluoreneamines or -diamines, and indenofluorene derivatives having fused aryl groups. Likewise preferred are pyrenearylamines. Likewise preferred are benzoindenofluoreneamines, benzofluoreneamines, extended benzoindenofluorenes, phenoxazines, and fluorene derivatives joined to furan units or to thiophene units. Examples of fluorescent emitters are depicted in the following table:

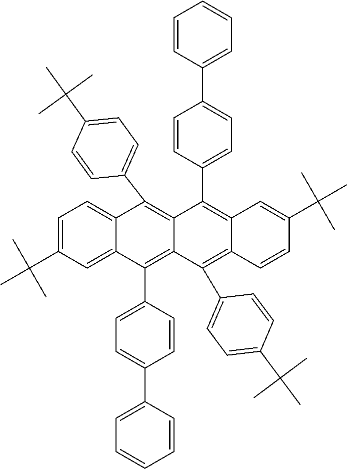 [SMILES: CC(C)(C)c1ccc(-c2c3ccc(C(C)(C)C)cc3c(-c3ccc(-c4ccccc4)cc3)c3c(-c4ccc(C(C)(C)C)cc4)c4ccc(C(C)(C)C)cc4c(-c4ccc(-c5ccccc5)cc4)c23)cc1]

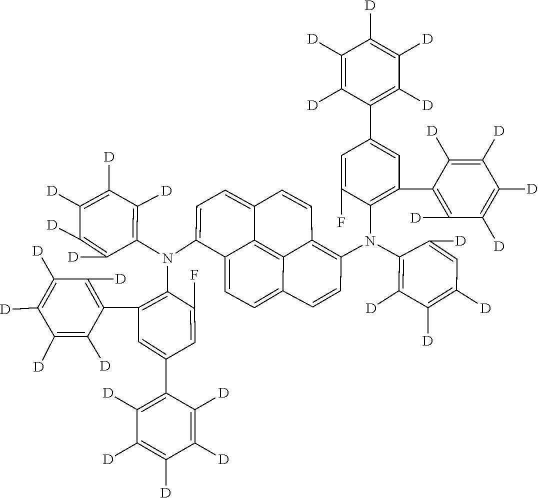 [SMILES: [2H]c1cc([2H])c(N(c2c(F)cc(-c3c([2H])c([2H])c([2H])c([2H])c3[2H])cc2-c2c([2H])c([2H])c([2H])c([2H])c2[2H])c2ccc3ccc4c(N(c5c(F)cc(-c6c([2H])c([2H])c([2H])c([2H])c6[2H])cc5-c5c([2H])c([2H])c([2H])c([2H])c5[2H])c5c([2H])c([2H])c([2H])c([2H])c5[2H])ccc5ccc2c3c54)c([2H])c1[2H]]

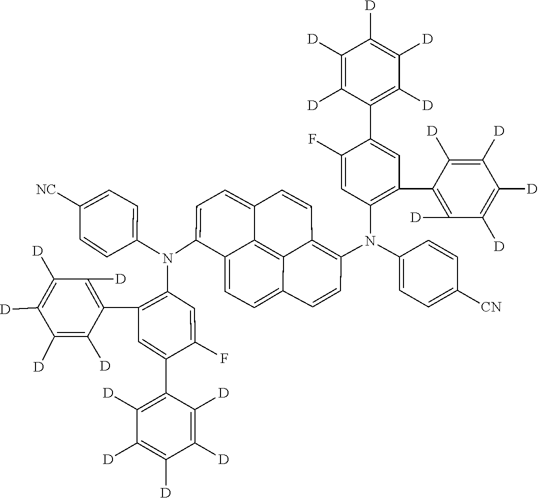 [SMILES: [2H]c1c([2H])c([2H])c(-c2cc(-c3c([2H])c([2H])c([2H])c([2H])c3[2H])c(N(c3ccc(C#N)cc3)c3ccc4ccc5c(N(c6ccc(C#N)cc6)c6cc(F)c(-c7c([2H])c([2H])c([2H])c([2H])c7[2H])cc6-c6c([2H])c([2H])c([2H])c([2H])c6[2H])ccc6ccc3c4c65)cc2F)c([2H])c1[2H]]

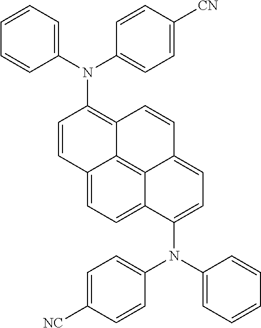 [SMILES: N#Cc1ccc(N(c2ccccc2)c2ccc3ccc4c(N(c5ccccc5)c5ccc(C#N)cc5)ccc5ccc2c3c54)cc1]

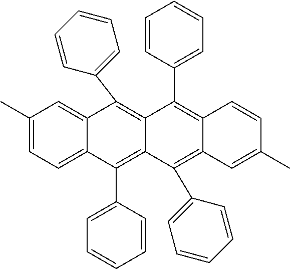

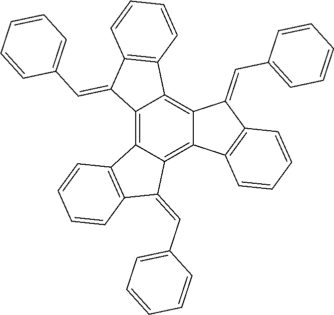 [SMILES: C(=C1/c2ccccc2-c2c1c1c(c3c2/C(=C/c2ccccc2)c2ccccc2-3)/C(=C/c2ccccc2)c2ccccc2-1)/c1ccccc1]

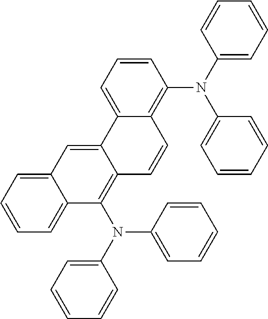 [SMILES: c1ccc(N(c2ccccc2)c2cccc3c2ccc2c(N(c4ccccc4)c4ccccc4)c4ccccc4cc23)cc1]

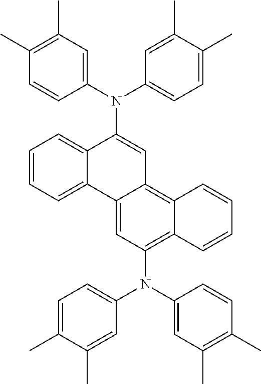

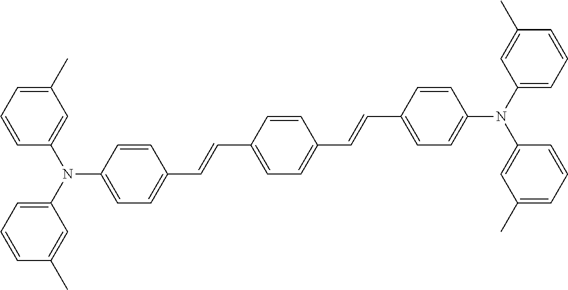 [SMILES: Cc1cccc(N(c2ccc(/C=C/c3ccc(/C=C/c4ccc(N(c5cccc(C)c5)c5cccc(C)c5)cc4)cc3)cc2)c2cccc(C)c2)c1]

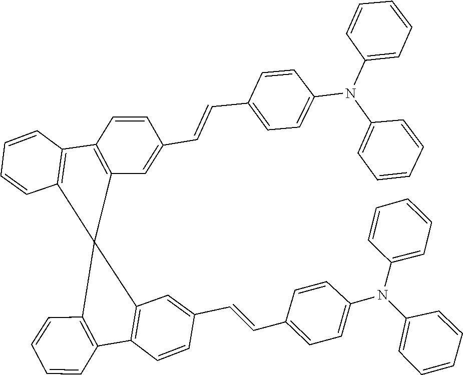 [SMILES: C(=C\c1ccc2c(c1)C1(c3ccccc3-2)c2ccccc2-c2ccc(/C=C/c3ccc(N(c4ccccc4)c4ccccc4)cc3)cc21)/c1ccc(N(c2ccccc2)c2ccccc2)cc1]

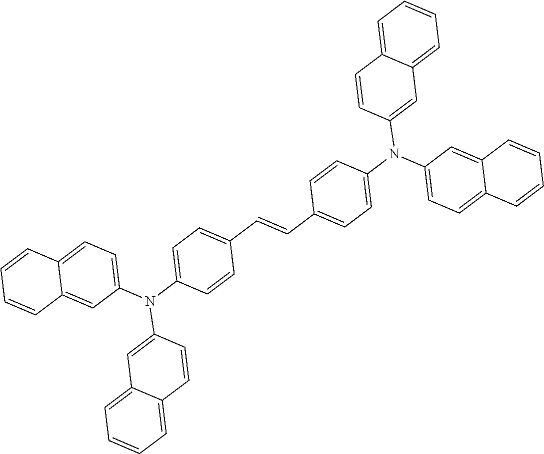 [SMILES: C(=C\c1ccc(N(c2ccc3ccccc3c2)c2ccc3ccccc3c2)cc1)/c1ccc(N(c2ccc3ccccc3c2)c2ccc3ccccc3c2)cc1]

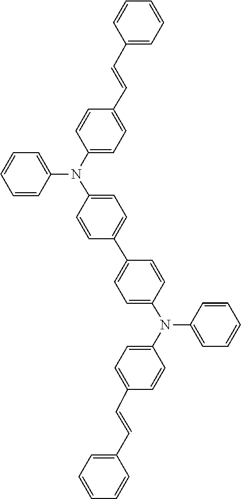 [SMILES: C(=C\c1ccc(N(c2ccccc2)c2ccc(-c3ccc(N(c4ccccc4)c4ccc(/C=C/c5ccccc5)cc4)cc3)cc2)cc1)/c1ccccc1]

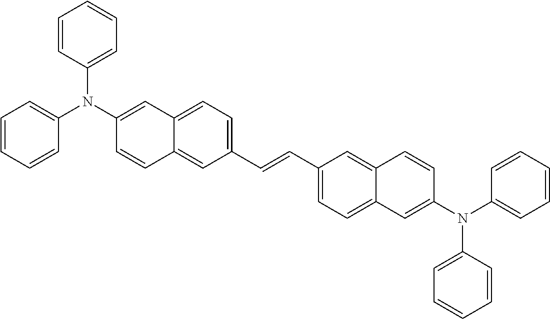 [SMILES: C(=C\c1ccc2cc(N(c3ccccc3)c3ccccc3)ccc2c1)/c1ccc2cc(N(c3ccccc3)c3ccccc3)ccc2c1]

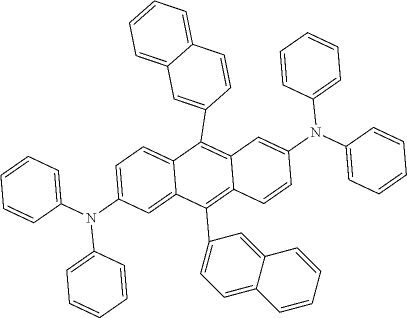 [SMILES: c1ccc(N(c2ccccc2)c2ccc3c(-c4ccc5ccccc5c4)c4cc(N(c5ccccc5)c5ccccc5)ccc4c(-c4ccc5ccccc5c4)c3c2)cc1]

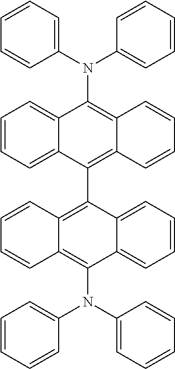 [SMILES: c1ccc(N(c2ccccc2)c2c3ccccc3c(-c3c4ccccc4c(N(c4ccccc4)c4ccccc4)c4ccccc34)c3ccccc23)cc1]

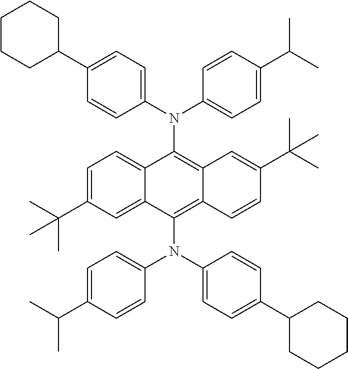 [SMILES: CC(C)c1ccc(N(c2ccc(C3CCCCC3)cc2)c2c3ccc(C(C)(C)C)cc3c(N(c3ccc(C(C)C)cc3)c3ccc(C4CCCCC4)cc3)c3ccc(C(C)(C)C)cc23)cc1]

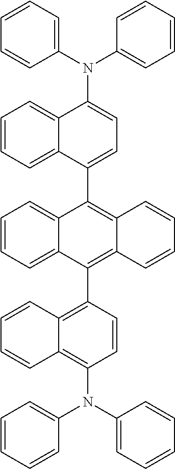 [SMILES: c1ccc(N(c2ccccc2)c2ccc(-c3c4ccccc4c(-c4ccc(N(c5ccccc5)c5ccccc5)c5ccccc45)c4ccccc34)c3ccccc23)cc1]

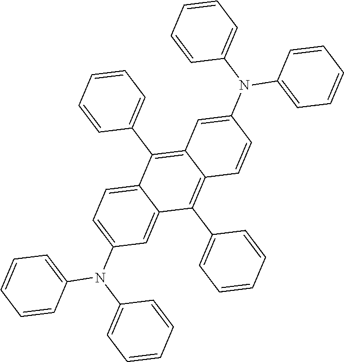 [SMILES: c1ccc(-c2c3ccc(N(c4ccccc4)c4ccccc4)cc3c(-c3ccccc3)c3ccc(N(c4ccccc4)c4ccccc4)cc23)cc1]

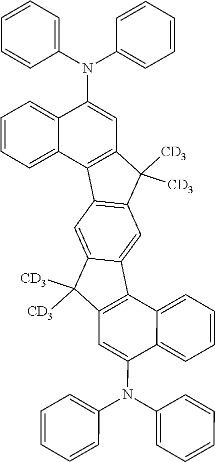 [SMILES: [2H]C([2H])([2H])C1(C([2H])([2H])[2H])c2cc3c(cc2-c2c1cc(N(c1ccccc1)c1ccccc1)c1ccccc21)C(C([2H])([2H])[2H])(C([2H])([2H])[2H])c1cc(N(c2ccccc2)c2ccccc2)c2ccccc2c1-3]

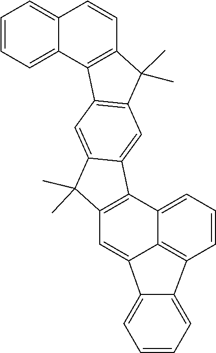 [SMILES: CC1(C)c2cc3c(cc2-c2c1ccc1ccccc21)C(C)(C)c1cc2c4c(cccc4c1-3)-c1ccccc1-2]

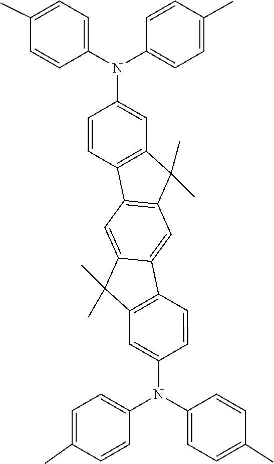

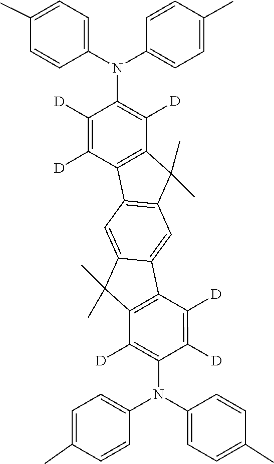

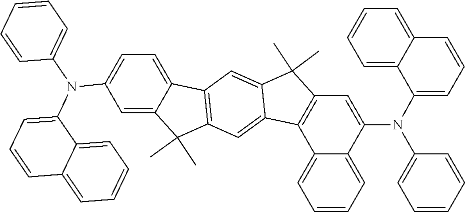 [SMILES: CC1(C)c2cc(N(c3ccccc3)c3cccc4ccccc34)ccc2-c2cc3c(cc21)-c1c(cc(N(c2ccccc2)c2cccc4ccccc24)c2ccccc12)C3(C)C]

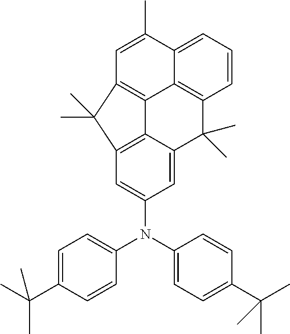 [SMILES: Cc1cc2c3c4c(cccc14)C(C)(C)c1cc(N(c4ccc(C(C)(C)C)cc4)c4ccc(C(C)(C)C)cc4)cc(c1-3)C2(C)C]

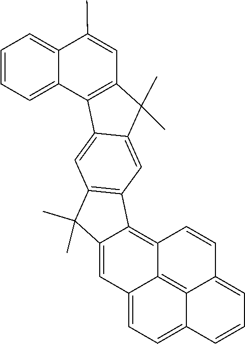 [SMILES: Cc1cc2c(c3ccccc13)-c1cc3c(cc1C2(C)C)-c1c(cc2ccc4cccc5ccc1c2c45)C3(C)C]

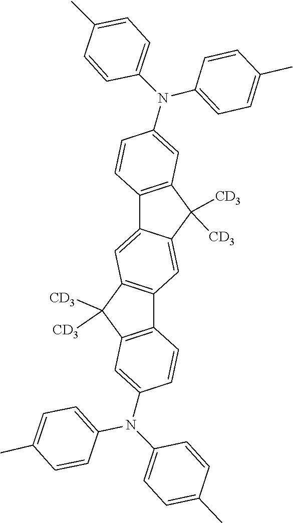

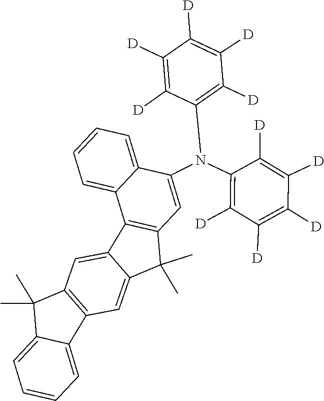 [SMILES: [2H]c1c([2H])c([2H])c(N(c2c([2H])c([2H])c([2H])c([2H])c2[2H])c2cc3c(c4ccccc24)-c2cc4c(cc2C3(C)C)-c2ccccc2C4(C)C)c([2H])c1[2H]]

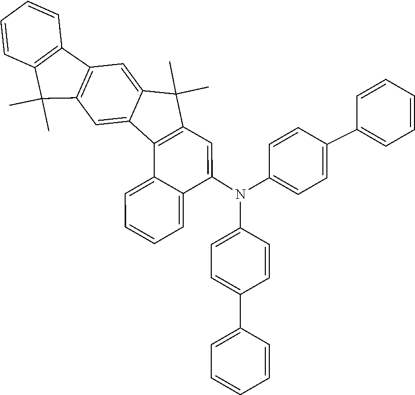 [SMILES: CC1(C)c2ccccc2-c2cc3c(cc21)-c1c(cc(N(c2ccc(-c4ccccc4)cc2)c2ccc(-c4ccccc4)cc2)c2ccccc12)C3(C)C]

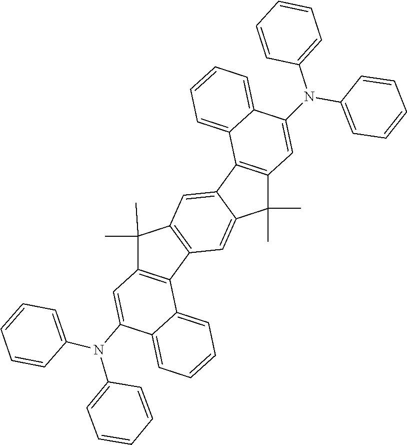 [SMILES: CC1(C)c2cc3c(cc2-c2c1cc(N(c1ccccc1)c1ccccc1)c1ccccc21)C(C)(C)c1cc(N(c2ccccc2)c2ccccc2)c2ccccc2c1-3]

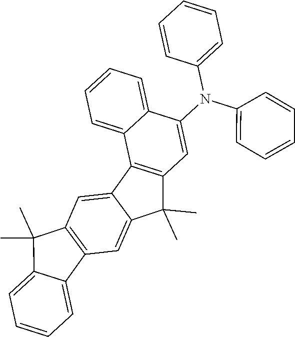 [SMILES: CC1(C)c2ccccc2-c2cc3c(cc21)-c1c(cc(N(c2ccccc2)c2ccccc2)c2ccccc12)C3(C)C]

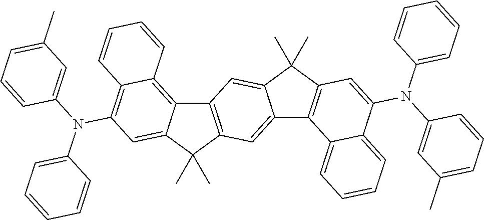 [SMILES: Cc1cccc(N(c2ccccc2)c2cc3c(c4ccccc24)-c2cc4c(cc2C3(C)C)-c2c(cc(N(c3ccccc3)c3cccc(C)c3)c3ccccc23)C4(C)C)c1]

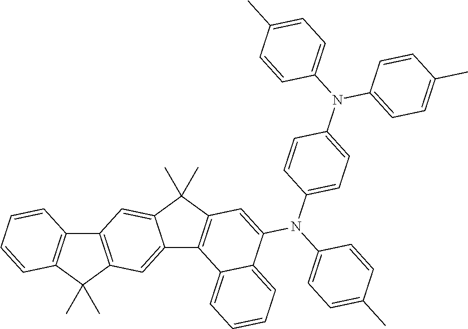 [SMILES: Cc1ccc(N(c2ccc(C)cc2)c2ccc(N(c3ccc(C)cc3)c3cc4c(c5ccccc35)-c3cc5c(cc3C4(C)C)-c3ccccc3C5(C)C)cc2)cc1]

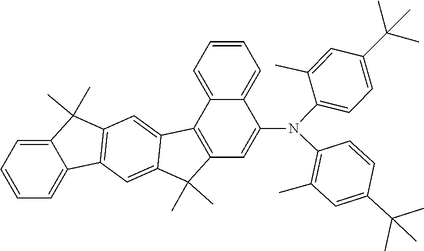 [SMILES: Cc1cc(C(C)(C)C)ccc1N(c1ccc(C(C)(C)C)cc1C)c1cc2c(c3ccccc13)-c1cc3c(cc1C2(C)C)-c1ccccc1C3(C)C]

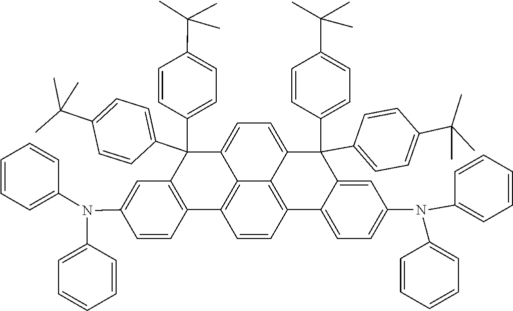 [SMILES: CC(C)(C)c1ccc(C2(c3ccc(C(C)(C)C)cc3)c3cc(N(c4ccccc4)c4ccccc4)ccc3-c3ccc4c5c(ccc2c35)C(c2ccc(C(C)(C)C)cc2)(c2ccc(C(C)(C)C)cc2)c2cc(N(c3ccccc3)c3ccccc3)ccc2-4)cc1]

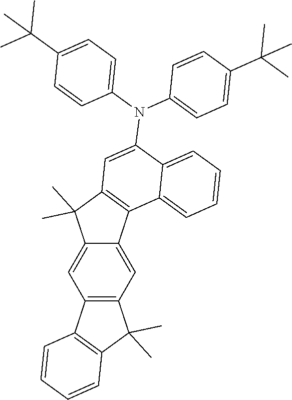 [SMILES: CC(C)(C)c1ccc(N(c2ccc(C(C)(C)C)cc2)c2cc3c(c4ccccc24)-c2cc4c(cc2C3(C)C)-c2ccccc2C4(C)C)cc1]

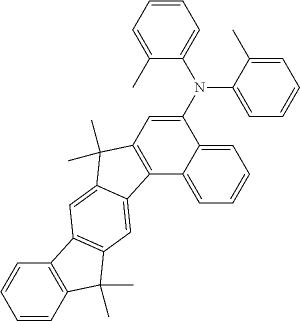 [SMILES: Cc1ccccc1N(c1ccccc1C)c1cc2c(c3ccccc13)-c1cc3c(cc1C2(C)C)-c1ccccc1C3(C)C]

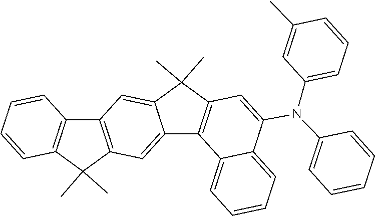 [SMILES: Cc1cccc(N(c2ccccc2)c2cc3c(c4ccccc24)-c2cc4c(cc2C3(C)C)-c2ccccc2C4(C)C)c1]

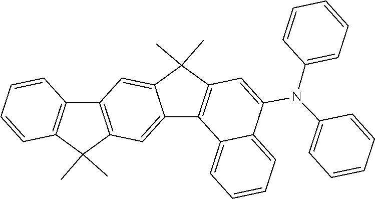 [SMILES: CC1(C)c2ccccc2-c2cc3c(cc21)-c1c(cc(N(c2ccccc2)c2ccccc2)c2ccccc12)C3(C)C]

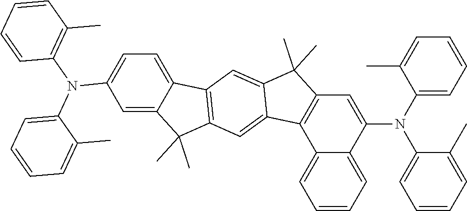 [SMILES: Cc1ccccc1N(c1ccc2c(c1)C(C)(C)c1cc3c(cc1-2)C(C)(C)c1cc(N(c2ccccc2C)c2ccccc2C)c2ccccc2c1-3)c1ccccc1C]

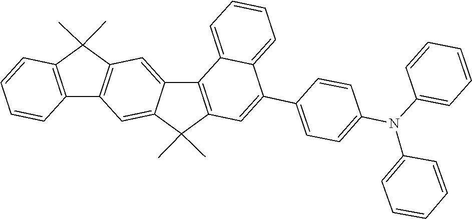 [SMILES: CC1(C)c2ccccc2-c2cc3c(cc21)-c1c(cc(-c2ccc(N(c4ccccc4)c4ccccc4)cc2)c2ccccc12)C3(C)C]

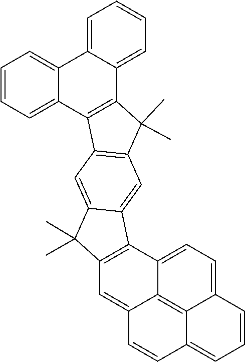 [SMILES: CC1(C)c2cc3c(cc2-c2c1cc1ccc4cccc5ccc2c1c45)C(C)(C)c1c-3c2ccccc2c2ccccc12]

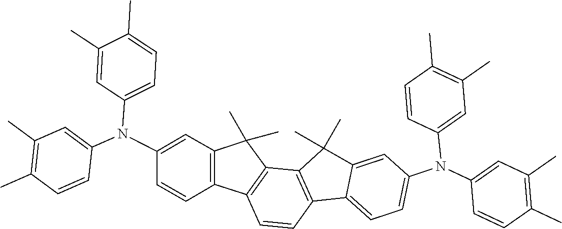

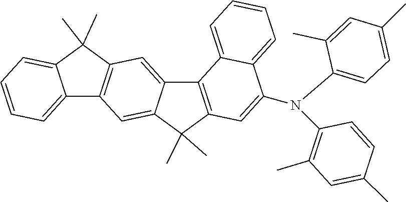 [SMILES: Cc1ccc(N(c2ccc(C)cc2C)c2cc3c(c4ccccc24)-c2cc4c(cc2C3(C)C)-c2ccccc2C4(C)C)c(C)c1]

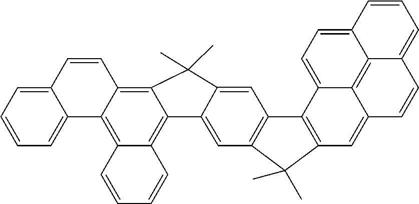 [SMILES: CC1(C)c2cc3c(cc2-c2c1cc1ccc4cccc5ccc2c1c45)C(C)(C)c1c-3c2ccccc2c2c1ccc1ccccc12]

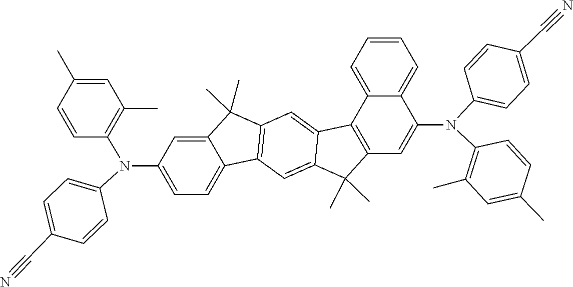 [SMILES: Cc1ccc(N(c2ccc(C#N)cc2)c2ccc3c(c2)C(C)(C)c2cc4c(cc2-3)C(C)(C)c2cc(N(c3ccc(C#N)cc3)c3ccc(C)cc3C)c3ccccc3c2-4)c(C)c1]

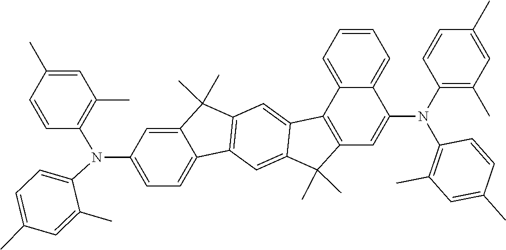

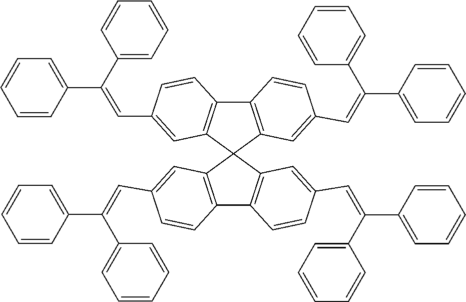 [SMILES: C(=C(c1ccccc1)c1ccccc1)c1ccc2c(c1)C1(c3cc(C=C(c4ccccc4)c4ccccc4)ccc3-2)c2cc(C=C(c3ccccc3)c3ccccc3)ccc2-c2ccc(C=C(c3ccccc3)c3ccccc3)cc21]

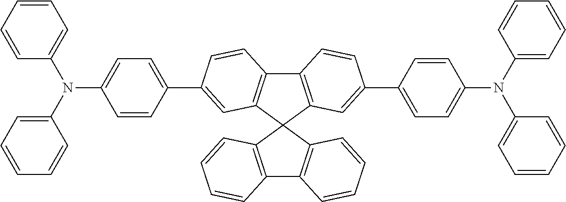 [SMILES: c1ccc(N(c2ccccc2)c2ccc(-c3ccc4c(c3)C3(c5ccccc5-c5ccccc53)c3cc(-c5ccc(N(c6ccccc6)c6ccccc6)cc5)ccc3-4)cc2)cc1]

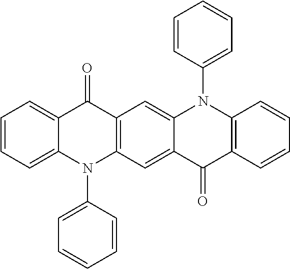 [SMILES: O=c1c2ccccc2n(-c2ccccc2)c2cc3c(=O)c4ccccc4n(-c4ccccc4)c3cc12]

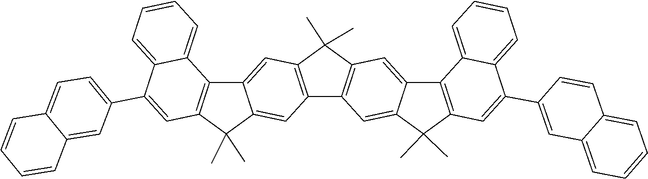 [SMILES: CC1(C)c2cc3c(cc2-c2cc4c(cc21)-c1c(cc(-c2ccc5ccccc5c2)c2ccccc12)C4(C)C)C(C)(C)c1cc(-c2ccc4ccccc4c2)c2ccccc2c1-3]

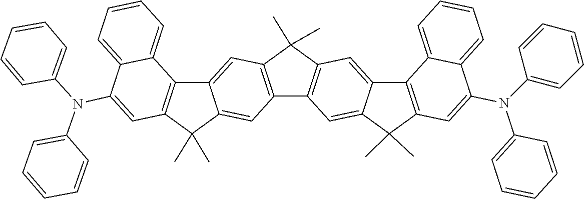 [SMILES: CC1(C)c2cc3c(cc2-c2cc4c(cc21)-c1c(cc(N(c2ccccc2)c2ccccc2)c2ccccc12)C4(C)C)C(C)(C)c1cc(N(c2ccccc2)c2ccccc2)c2ccccc2c1-3]

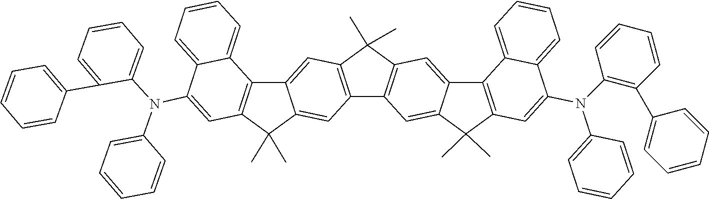 [SMILES: CC1(C)c2cc3c(cc2-c2cc4c(cc21)-c1c(cc(N(c2ccccc2)c2ccccc2-c2ccccc2)c2ccccc12)C4(C)C)C(C)(C)c1cc(N(c2ccccc2)c2ccccc2-c2ccccc2)c2ccccc2c1-3]

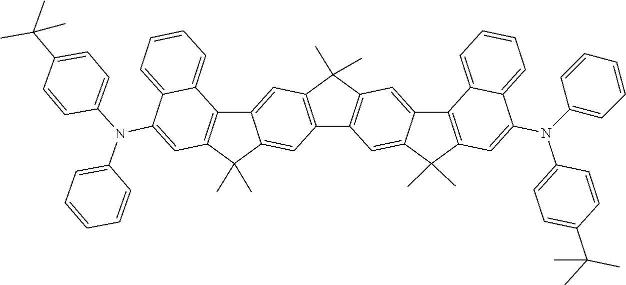 [SMILES: CC(C)(C)c1ccc(N(c2ccccc2)c2cc3c(c4ccccc24)-c2cc4c(cc2C3(C)C)-c2cc3c(cc2C4(C)C)-c2c(cc(N(c4ccccc4)c4ccc(C(C)(C)C)cc4)c4ccccc24)C3(C)C)cc1]

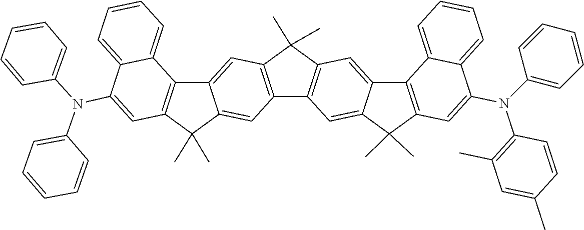 [SMILES: Cc1ccc(N(c2ccccc2)c2cc3c(c4ccccc24)-c2cc4c(cc2C3(C)C)-c2cc3c(cc2C4(C)C)-c2c(cc(N(c4ccccc4)c4ccccc4)c4ccccc24)C3(C)C)c(C)c1]

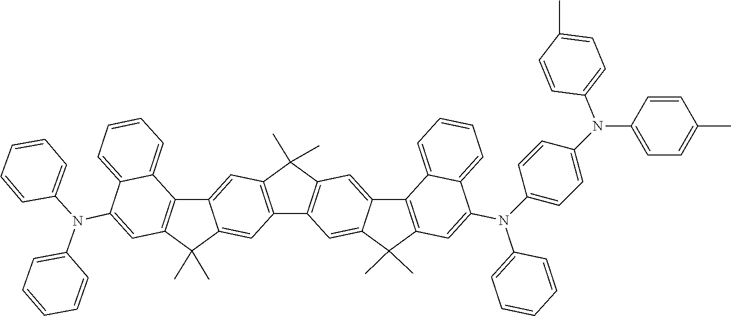 [SMILES: Cc1ccc(N(c2ccc(C)cc2)c2ccc(N(c3ccccc3)c3cc4c(c5ccccc35)-c3cc5c(cc3C4(C)C)-c3cc4c(cc3C5(C)C)-c3c(cc(N(c5ccccc5)c5ccccc5)c5ccccc35)C4(C)C)cc2)cc1]

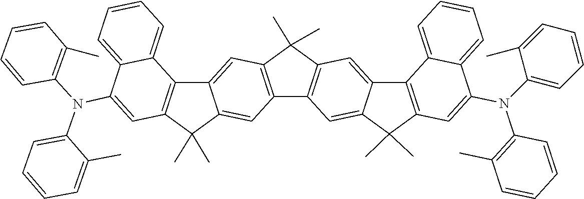 [SMILES: Cc1ccccc1N(c1ccccc1C)c1cc2c(c3ccccc13)-c1cc3c(cc1C2(C)C)-c1cc2c(cc1C3(C)C)-c1c(cc(N(c3ccccc3C)c3ccccc3C)c3ccccc13)C2(C)C]

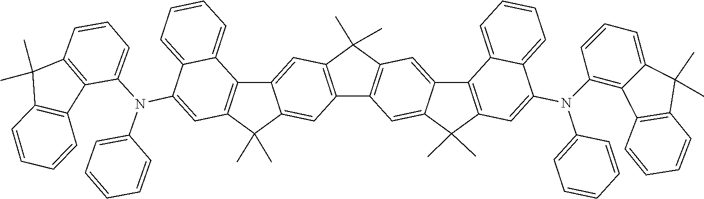

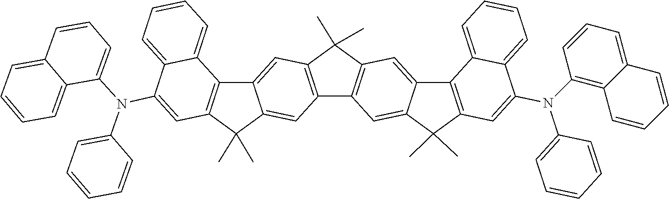 [SMILES: CC1(C)c2cc3c(cc2-c2cc4c(cc21)-c1c(cc(N(c2ccccc2)c2cccc5ccccc25)c2ccccc12)C4(C)C)C(C)(C)c1cc(N(c2ccccc2)c2cccc4ccccc24)c2ccccc2c1-3]

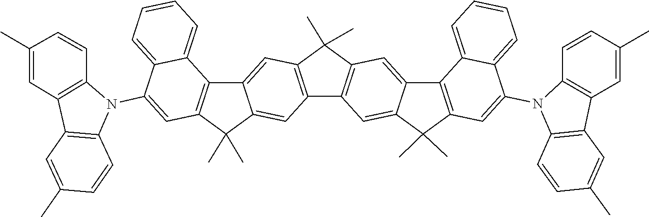

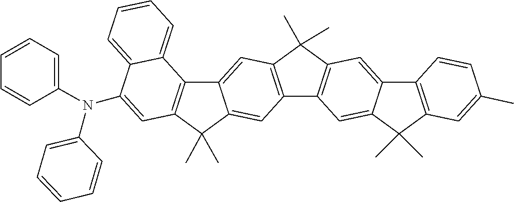 [SMILES: Cc1ccc2c(c1)C(C)(C)c1cc3c(cc1-2)C(C)(C)c1cc2c(cc1-3)C(C)(C)c1cc(N(c3ccccc3)c3ccccc3)c3ccccc3c1-2]

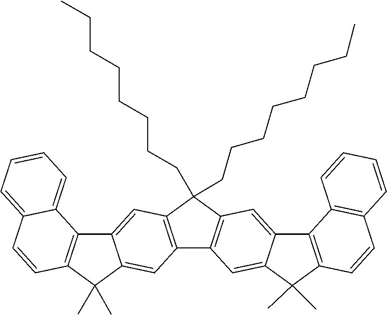 [SMILES: CCCCCCCCC1(CCCCCCCC)c2cc3c(cc2-c2cc4c(cc21)-c1c(ccc2ccccc12)C4(C)C)C(C)(C)c1ccc2ccccc2c1-3]

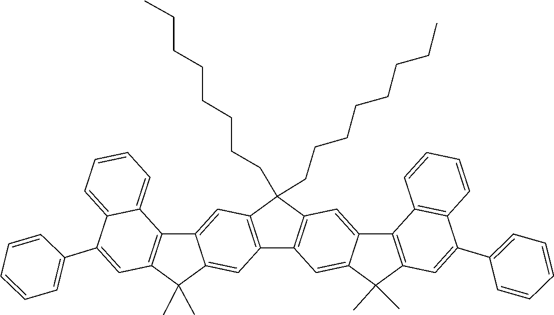 [SMILES: CCCCCCCCC1(CCCCCCCC)c2cc3c(cc2-c2cc4c(cc21)-c1c(cc(-c2ccccc2)c2ccccc12)C4(C)C)C(C)(C)c1cc(-c2ccccc2)c2ccccc2c1-3]

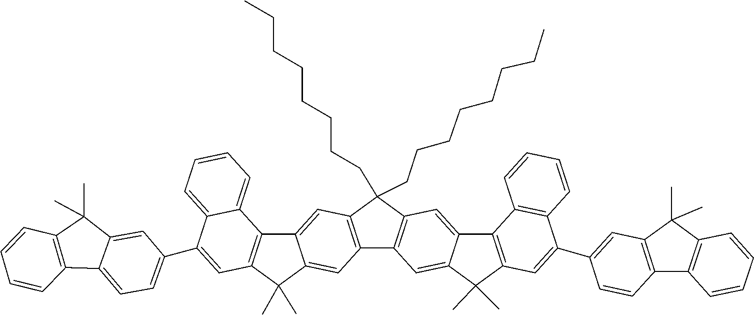 [SMILES: CCCCCCCCC1(CCCCCCCC)c2cc3c(cc2-c2cc4c(cc21)-c1c(cc(-c2ccc5c(c2)C(C)(C)c2ccccc2-5)c2ccccc12)C4(C)C)C(C)(C)c1cc(-c2ccc4c(c2)C(C)(C)c2ccccc2-4)c2ccccc2c1-3]

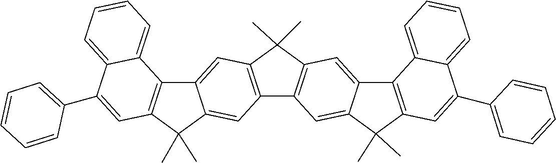 [SMILES: CC1(C)c2cc3c(cc2-c2cc4c(cc21)-c1c(cc(-c2ccccc2)c2ccccc12)C4(C)C)C(C)(C)c1cc(-c2ccccc2)c2ccccc2c1-3]

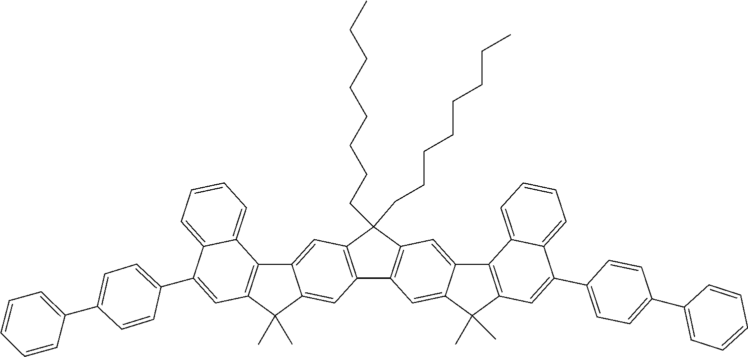 [SMILES: CCCCCCCCC1(CCCCCCCC)c2cc3c(cc2-c2cc4c(cc21)-c1c(cc(-c2ccc(-c5ccccc5)cc2)c2ccccc12)C4(C)C)C(C)(C)c1cc(-c2ccc(-c4ccccc4)cc2)c2ccccc2c1-3]

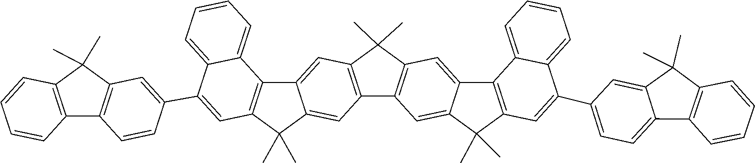 [SMILES: CC1(C)c2ccccc2-c2ccc(-c3cc4c(c5ccccc35)-c3cc5c(cc3C4(C)C)-c3cc4c(cc3C5(C)C)-c3c(cc(-c5ccc6c(c5)C(C)(C)c5ccccc5-6)c5ccccc35)C4(C)C)cc21]

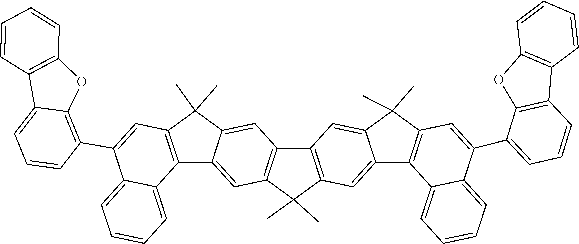 [SMILES: CC1(C)c2cc3c(cc2-c2cc4c(cc21)-c1c(cc(-c2cccc5c2oc2ccccc25)c2ccccc12)C4(C)C)C(C)(C)c1cc(-c2cccc4c2oc2ccccc24)c2ccccc2c1-3]

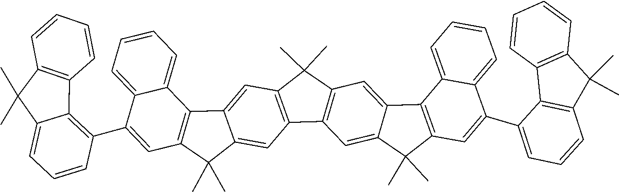

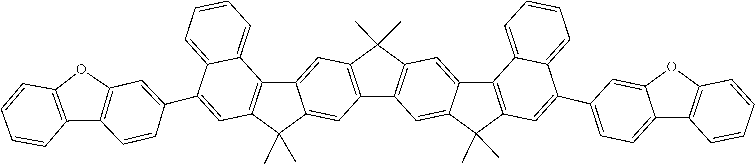 [SMILES: CC1(C)c2cc3c(cc2-c2cc4c(cc21)-c1c(cc(-c2ccc5c(c2)oc2ccccc25)c2ccccc12)C4(C)C)C(C)(C)c1cc(-c2ccc4c(c2)oc2ccccc24)c2ccccc2c1-3]

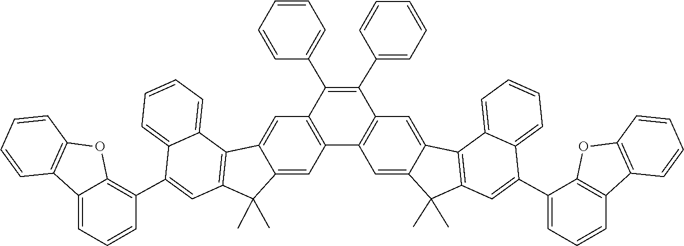 [SMILES: CC1(C)c2cc3c(cc2-c2c1cc(-c1cccc4c1oc1ccccc14)c1ccccc21)c(-c1ccccc1)c(-c1ccccc1)c1cc2c(cc13)C(C)(C)c1cc(-c3cccc4c3oc3ccccc34)c3ccccc3c1-2]

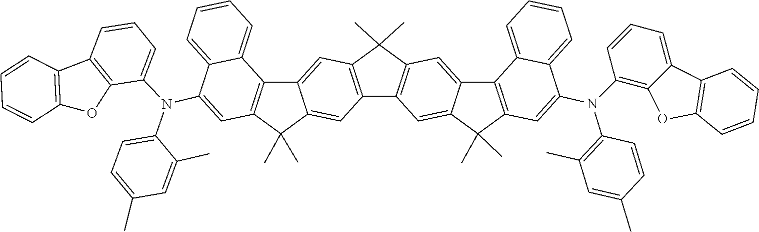 [SMILES: Cc1ccc(N(c2cc3c(c4ccccc24)-c2cc4c(cc2C3(C)C)-c2cc3c(cc2C4(C)C)-c2c(cc(N(c4ccc(C)cc4C)c4cccc5c4oc4ccccc45)c4ccccc24)C3(C)C)c2cccc3c2oc2ccccc23)c(C)c1]

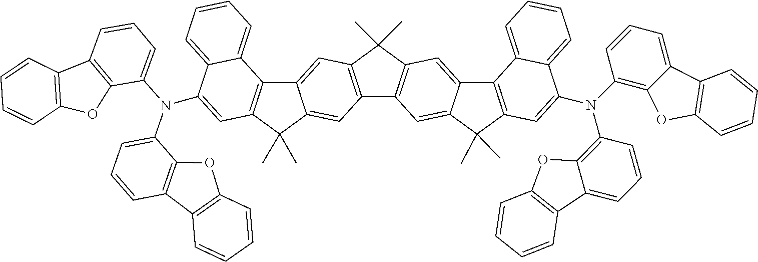 [SMILES: CC1(C)c2cc3c(cc2-c2cc4c(cc21)-c1c(cc(N(c2cccc5c2oc2ccccc25)c2cccc5c2oc2ccccc25)c2ccccc12)C4(C)C)C(C)(C)c1cc(N(c2cccc4c2oc2ccccc24)c2cccc4c2oc2ccccc24)c2ccccc2c1-3]

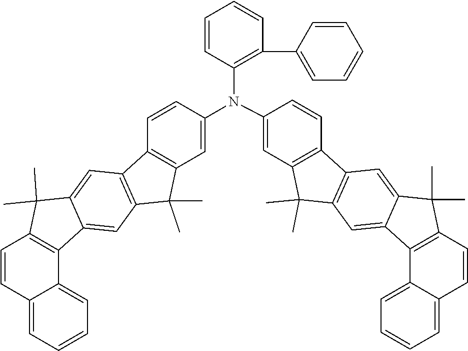 [SMILES: CC1(C)c2cc(N(c3ccc4c(c3)C(C)(C)c3cc5c(cc3-4)C(C)(C)c3ccc4ccccc4c3-5)c3ccccc3-c3ccccc3)ccc2-c2cc3c(cc21)-c1c(ccc2ccccc12)C3(C)C]

Matrix Materials for Fluorescent Emitters:

Preferred matrix materials for fluorescent emitters are selected from the classes of the oligoarylenes (e.g. 2,2′,7,7′-tetraphenylspirobifluorene), especially the oligoarylenes containing fused aromatic groups, the oligoarylenevinylenes, the polypodal metal complexes, the hole-conducting compounds, the electron-conducting compounds, especially ketones, phosphine oxides and sulfoxides; the atropisomers, the boronic acid derivatives or the benzanthracenes. Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoarylene in the context of this invention shall be understood to mean a compound in which at least three aryl or arylene groups are bonded to one another. Preferred matrix materials for fluorescent emitters are depicted in the following table:

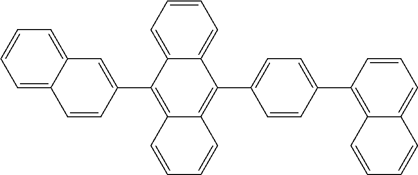 [SMILES: c1ccc2cc(-c3c4ccccc4c(-c4ccc(-c5cccc6ccccc56)cc4)c4ccccc34)ccc2c1]

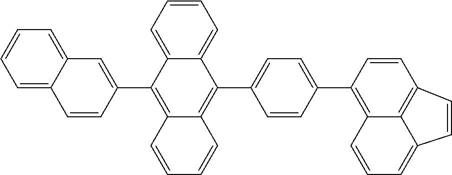 [SMILES: C1=Cc2ccc(-c3ccc(-c4c5ccccc5c(-c5ccc6ccccc6c5)c5ccccc45)cc3)c3cccc1c23]

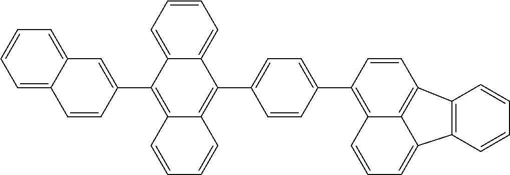 [SMILES: c1ccc2c(c1)-c1cccc3c(-c4ccc(-c5c6ccccc6c(-c6ccc7ccccc7c6)c6ccccc56)cc4)ccc-2c13]

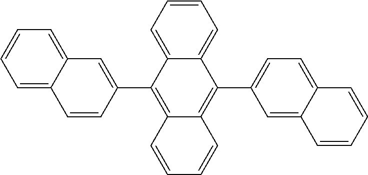 [SMILES: c1ccc2cc(-c3c4ccccc4c(-c4ccc5ccccc5c4)c4ccccc34)ccc2c1]

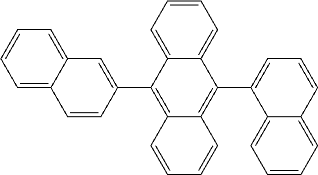 [SMILES: c1ccc2cc(-c3c4ccccc4c(-c4cccc5ccccc45)c4ccccc34)ccc2c1]

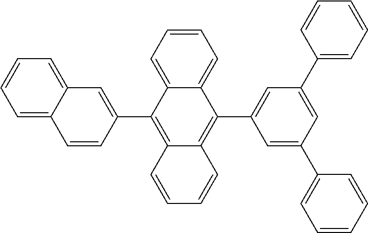 [SMILES: c1ccc(-c2cc(-c3ccccc3)cc(-c3c4ccccc4c(-c4ccc5ccccc5c4)c4ccccc34)c2)cc1]

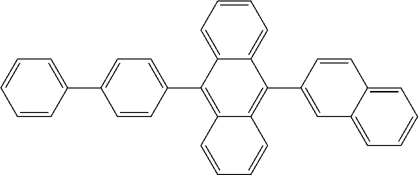 [SMILES: c1ccc(-c2ccc(-c3c4ccccc4c(-c4ccc5ccccc5c4)c4ccccc34)cc2)cc1]

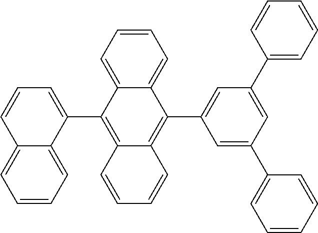 [SMILES: c1ccc(-c2cc(-c3ccccc3)cc(-c3c4ccccc4c(-c4cccc5ccccc45)c4ccccc34)c2)cc1]

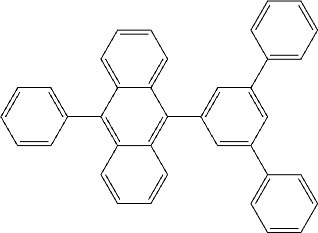 [SMILES: c1ccc(-c2cc(-c3ccccc3)cc(-c3c4ccccc4c(-c4ccccc4)c4ccccc34)c2)cc1]

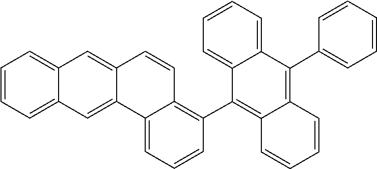 [SMILES: c1ccc(-c2c3ccccc3c(-c3cccc4c3ccc3cc5ccccc5cc34)c3ccccc23)cc1]

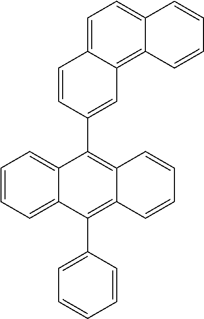 [SMILES: c1ccc(-c2c3ccccc3c(-c3ccc4ccc5ccccc5c4c3)c3ccccc23)cc1]

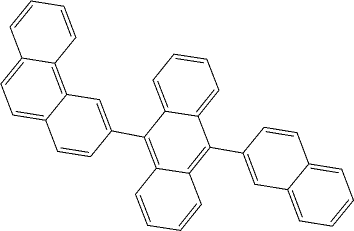 [SMILES: c1ccc2cc(-c3c4ccccc4c(-c4ccc5ccc6ccccc6c5c4)c4ccccc34)ccc2c1]

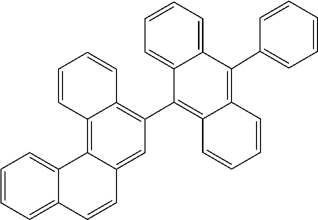 [SMILES: c1ccc(-c2c3ccccc3c(-c3cc4ccc5ccccc5c4c4ccccc34)c3ccccc23)cc1]

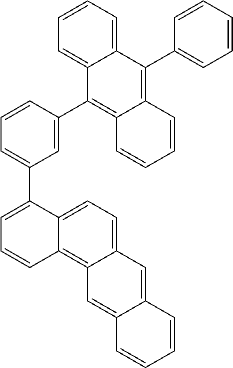 [SMILES: c1ccc(-c2c3ccccc3c(-c3cccc(-c4cccc5c4ccc4cc6ccccc6cc45)c3)c3ccccc23)cc1]

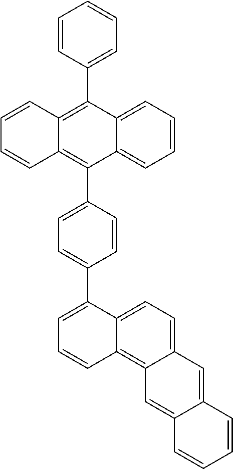 [SMILES: c1ccc(-c2c3ccccc3c(-c3ccc(-c4cccc5c4ccc4cc6ccccc6cc45)cc3)c3ccccc23)cc1]

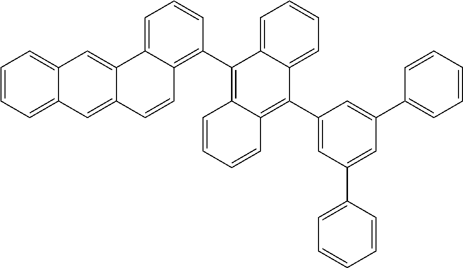 [SMILES: c1ccc(-c2cc(-c3ccccc3)cc(-c3c4ccccc4c(-c4cccc5c4ccc4cc6ccccc6cc45)c4ccccc34)c2)cc1]

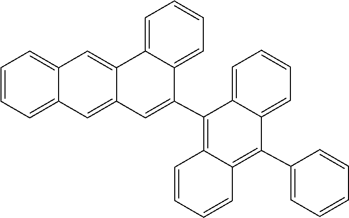 [SMILES: c1ccc(-c2c3ccccc3c(-c3cc4cc5ccccc5cc4c4ccccc34)c3ccccc23)cc1]

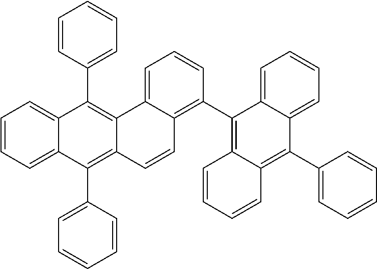 [SMILES: c1ccc(-c2c3ccccc3c(-c3cccc4c3ccc3c(-c5ccccc5)c5ccccc5c(-c5ccccc5)c34)c3ccccc23)cc1]

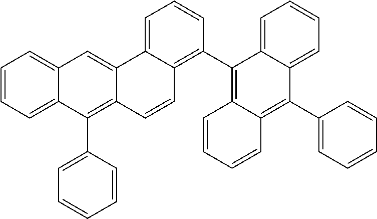 [SMILES: c1ccc(-c2c3ccccc3c(-c3cccc4c3ccc3c(-c5ccccc5)c5ccccc5cc34)c3ccccc23)cc1]

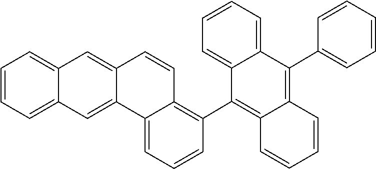 [SMILES: c1ccc(-c2c3ccccc3c(-c3cccc4c3ccc3cc5ccccc5cc34)c3ccccc23)cc1]

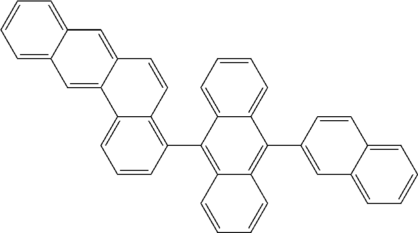 [SMILES: c1ccc2cc(-c3c4ccccc4c(-c4cccc5c4ccc4cc6ccccc6cc45)c4ccccc34)ccc2c1]

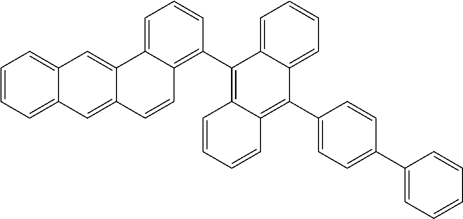 [SMILES: c1ccc(-c2ccc(-c3c4ccccc4c(-c4cccc5c4ccc4cc6ccccc6cc45)c4ccccc34)cc2)cc1]

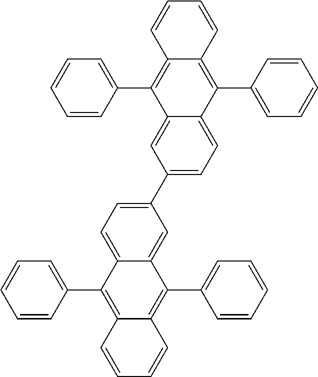 [SMILES: c1ccc(-c2c3ccccc3c(-c3ccccc3)c3cc(-c4ccc5c(-c6ccccc6)c6ccccc6c(-c6ccccc6)c5c4)ccc23)cc1]

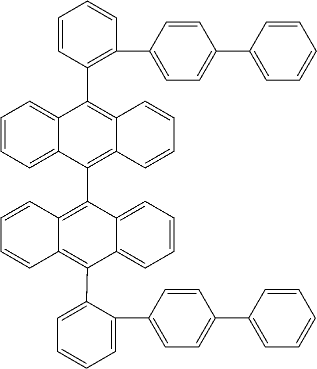 [SMILES: c1ccc(-c2ccc(-c3ccccc3-c3c4ccccc4c(-c4c5ccccc5c(-c5ccccc5-c5ccc(-c6ccccc6)cc5)c5ccccc45)c4ccccc34)cc2)cc1]

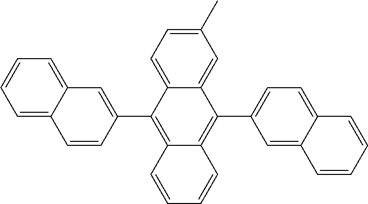 [SMILES: Cc1ccc2c(-c3ccc4ccccc4c3)c3ccccc3c(-c3ccc4ccccc4c3)c2c1]

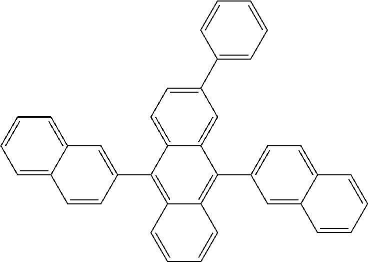 [SMILES: c1ccc(-c2ccc3c(-c4ccc5ccccc5c4)c4ccccc4c(-c4ccc5ccccc5c4)c3c2)cc1]

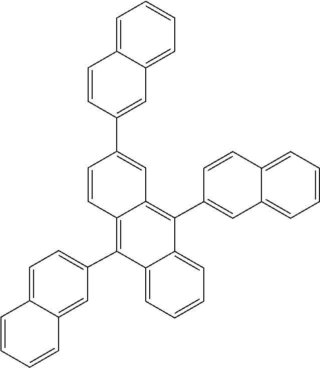 [SMILES: c1ccc2cc(-c3ccc4c(-c5ccc6ccccc6c5)c5ccccc5c(-c5ccc6ccccc6c5)c4c3)ccc2c1]

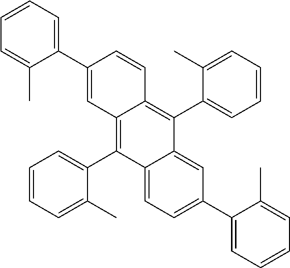 [SMILES: Cc1ccccc1-c1ccc2c(-c3ccccc3C)c3cc(-c4ccccc4C)ccc3c(-c3ccccc3C)c2c1]

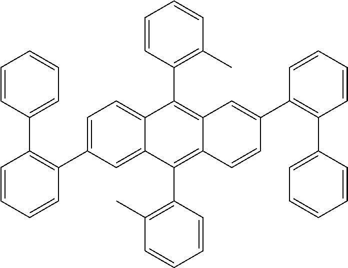 [SMILES: Cc1ccccc1-c1c2ccc(-c3ccccc3-c3ccccc3)cc2c(-c2ccccc2C)c2ccc(-c3ccccc3-c3ccccc3)cc12]

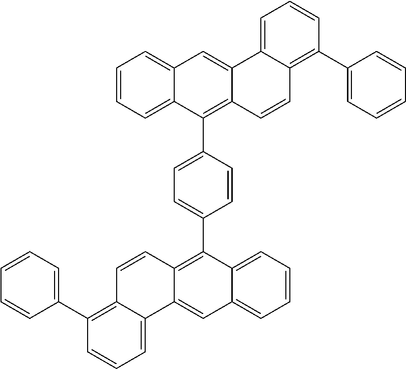 [SMILES: c1ccc(-c2cccc3c2ccc2c(-c4ccc(-c5c6ccccc6cc6c5ccc5c(-c7ccccc7)cccc56)cc4)c4ccccc4cc23)cc1]

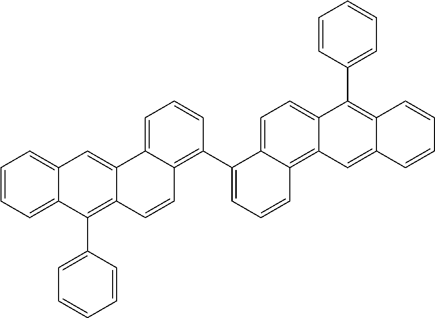 [SMILES: c1ccc(-c2c3ccccc3cc3c2ccc2c(-c4cccc5c4ccc4c(-c6ccccc6)c6ccccc6cc45)cccc23)cc1]

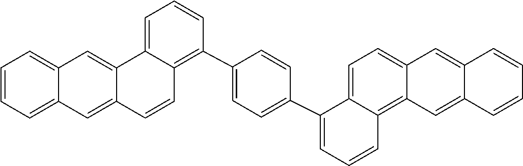 [SMILES: c1ccc2cc3c(ccc4c(-c5ccc(-c6cccc7c6ccc6cc8ccccc8cc67)cc5)cccc43)cc2c1]

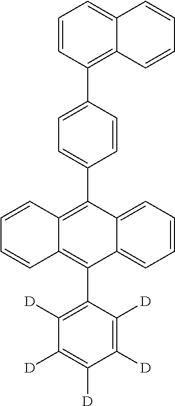 [SMILES: [2H]c1c([2H])c([2H])c(-c2c3ccccc3c(-c3ccc(-c4cccc5ccccc45)cc3)c3ccccc23)c([2H])c1[2H]]

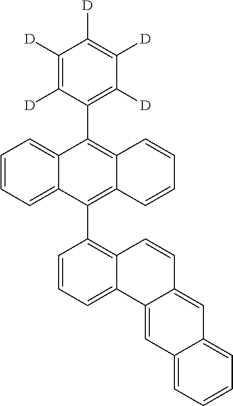 [SMILES: [2H]c1c([2H])c([2H])c(-c2c3ccccc3c(-c3cccc4c3ccc3cc5ccccc5cc34)c3ccccc23)c([2H])c1[2H]]

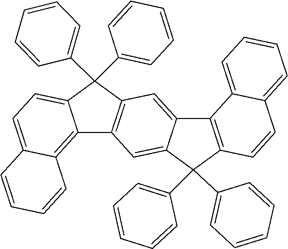 [SMILES: c1ccc(C2(c3ccccc3)c3cc4c(cc3-c3c2ccc2ccccc32)C(c2ccccc2)(c2ccccc2)c2ccc3ccccc3c2-4)cc1]

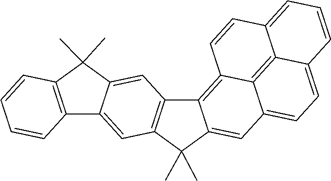 [SMILES: CC1(C)c2ccccc2-c2cc3c(cc21)-c1c(cc2ccc4cccc5ccc1c2c45)C3(C)C]

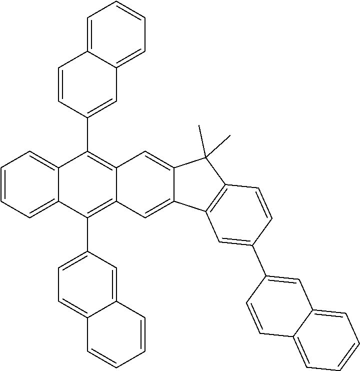 [SMILES: CC1(C)c2ccc(-c3ccc4ccccc4c3)cc2-c2cc3c(-c4ccc5ccccc5c4)c4ccccc4c(-c4ccc5ccccc5c4)c3cc21]

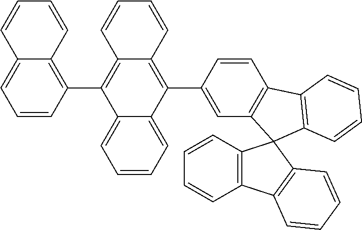 [SMILES: c1ccc2c(c1)-c1ccccc1C21c2ccccc2-c2ccc(-c3c4ccccc4c(-c4cccc5ccccc45)c4ccccc34)cc21]

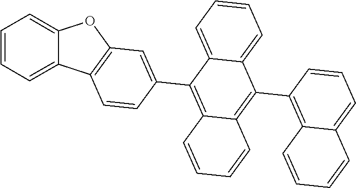 [SMILES: c1ccc2c(-c3c4ccccc4c(-c4ccc5c(c4)oc4ccccc45)c4ccccc34)cccc2c1]

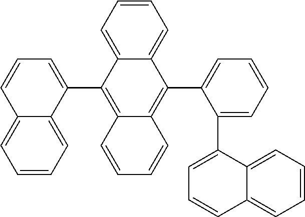 [SMILES: c1ccc(-c2c3ccccc3c(-c3cccc4ccccc34)c3ccccc23)c(-c2cccc3ccccc23)c1]

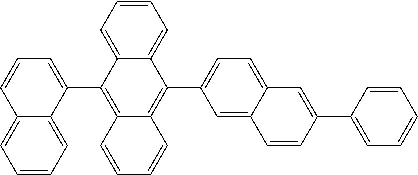 [SMILES: c1ccc(-c2ccc3cc(-c4c5ccccc5c(-c5cccc6ccccc56)c5ccccc45)ccc3c2)cc1]

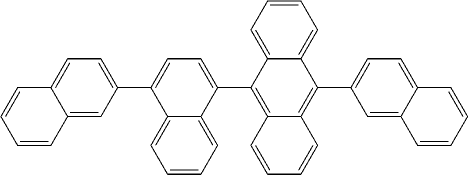 [SMILES: c1ccc2cc(-c3ccc(-c4c5ccccc5c(-c5ccc6ccccc6c5)c5ccccc45)c4ccccc34)ccc2c1]

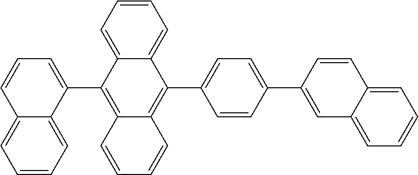 [SMILES: c1ccc2cc(-c3ccc(-c4c5ccccc5c(-c5cccc6ccccc56)c5ccccc45)cc3)ccc2c1]

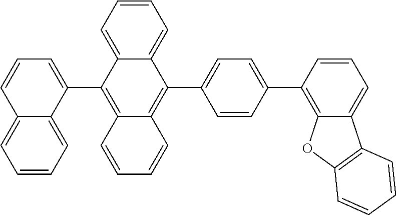 [SMILES: c1ccc2c(-c3c4ccccc4c(-c4ccc(-c5cccc6c5oc5ccccc56)cc4)c4ccccc34)cccc2c1]

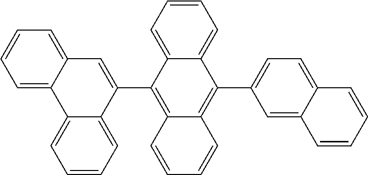 [SMILES: c1ccc2cc(-c3c4ccccc4c(-c4cc5ccccc5c5ccccc45)c4ccccc34)ccc2c1]

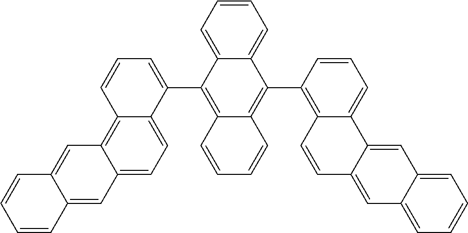 [SMILES: c1ccc2cc3c(ccc4c(-c5c6ccccc6c(-c6cccc7c6ccc6cc8ccccc8cc67)c6ccccc56)cccc43)cc2c1]

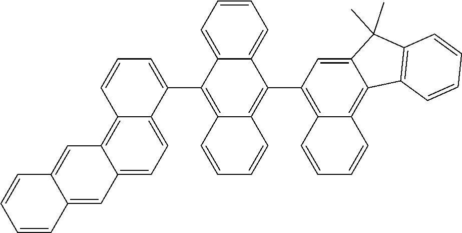 [SMILES: CC1(C)c2ccccc2-c2c1cc(-c1c3ccccc3c(-c3cccc4c3ccc3cc5ccccc5cc34)c3ccccc13)c1ccccc21]

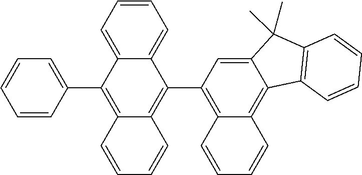 [SMILES: CC1(C)c2ccccc2-c2c1cc(-c1c3ccccc3c(-c3ccccc3)c3ccccc13)c1ccccc21]

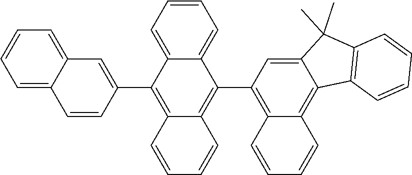 [SMILES: CC1(C)c2ccccc2-c2c1cc(-c1c3ccccc3c(-c3ccc4ccccc4c3)c3ccccc13)c1ccccc21]

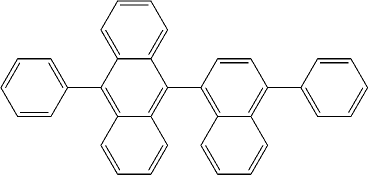 [SMILES: c1ccc(-c2ccc(-c3c4ccccc4c(-c4ccccc4)c4ccccc34)c3ccccc23)cc1]

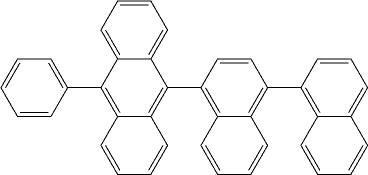 [SMILES: c1ccc(-c2c3ccccc3c(-c3ccc(-c4cccc5ccccc45)c4ccccc34)c3ccccc23)cc1]

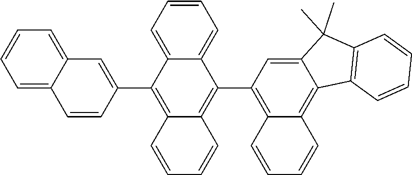 [SMILES: CC1(C)c2ccccc2-c2c1cc(-c1c3ccccc3c(-c3ccc4ccccc4c3)c3ccccc13)c1ccccc21]

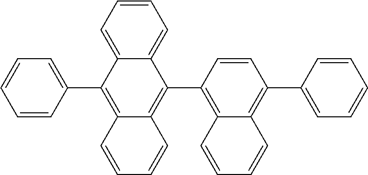 [SMILES: c1ccc(-c2ccc(-c3c4ccccc4c(-c4ccccc4)c4ccccc34)c3ccccc23)cc1]

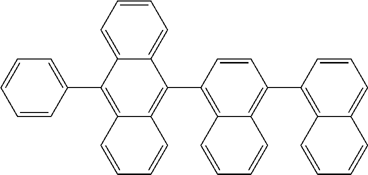 [SMILES: c1ccc(-c2c3ccccc3c(-c3ccc(-c4cccc5ccccc45)c4ccccc34)c3ccccc23)cc1]

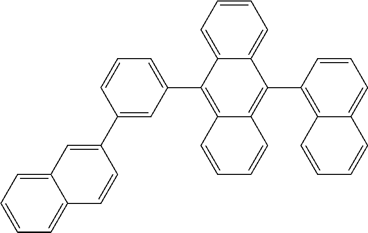 [SMILES: c1cc(-c2ccc3ccccc3c2)cc(-c2c3ccccc3c(-c3cccc4ccccc34)c3ccccc23)c1]

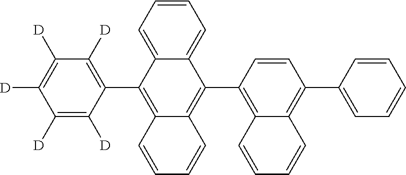 [SMILES: [2H]c1c([2H])c([2H])c(-c2c3ccccc3c(-c3ccc(-c4ccccc4)c4ccccc34)c3ccccc23)c([2H])c1[2H]]

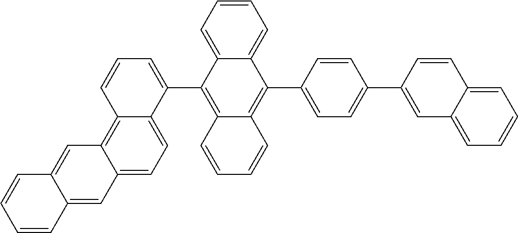 [SMILES: c1ccc2cc(-c3ccc(-c4c5ccccc5c(-c5cccc6c5ccc5cc7ccccc7cc56)c5ccccc45)cc3)ccc2c1]

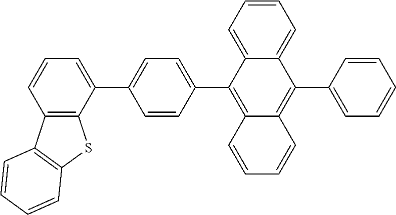 [SMILES: c1ccc(-c2c3ccccc3c(-c3ccc(-c4cccc5c4sc4ccccc45)cc3)c3ccccc23)cc1]

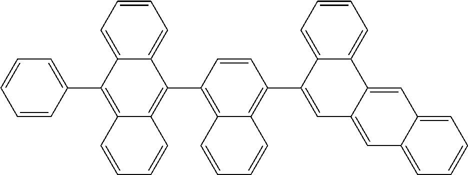 [SMILES: c1ccc(-c2c3ccccc3c(-c3ccc(-c4cc5cc6ccccc6cc5c5ccccc45)c4ccccc34)c3ccccc23)cc1]

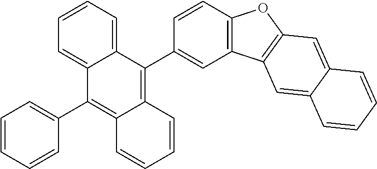 [SMILES: c1ccc(-c2c3ccccc3c(-c3ccc4oc5cc6ccccc6cc5c4c3)c3ccccc23)cc1]

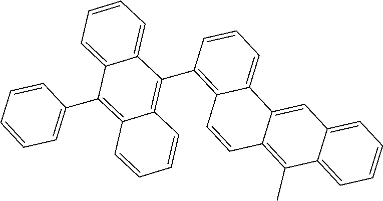 [SMILES: Cc1c2ccccc2cc2c1ccc1c(-c3c4ccccc4c(-c4ccccc4)c4ccccc34)cccc12]

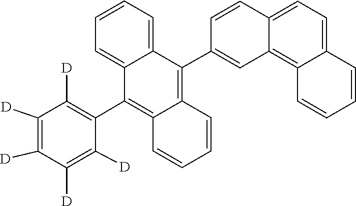 [SMILES: [2H]c1c([2H])c([2H])c(-c2c3ccccc3c(-c3ccc4ccc5ccccc5c4c3)c3ccccc23)c([2H])c1[2H]]

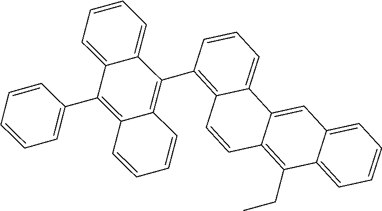 [SMILES: CCc1c2ccccc2cc2c1ccc1c(-c3c4ccccc4c(-c4ccccc4)c4ccccc34)cccc12]

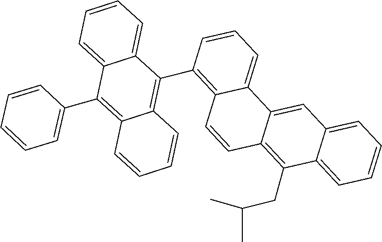 [SMILES: CC(C)Cc1c2ccccc2cc2c1ccc1c(-c3c4ccccc4c(-c4ccccc4)c4ccccc34)cccc12]

Matrix materials for phosphorescent emitters:

Preferred matrix materials for phosphorescent emitters are, as well as the compounds of the formula (1), aromatic ketones, aromatic phosphine oxides or aromatic sulfoxides or sulfones, triarylamines, carbazole derivatives, e.g. CBP (N,N-biscarbazolylbiphenyl), indolocarbazole derivatives, indenocarbazole derivatives, azacarbazole derivatives, bipolar matrix materials, silanes, azaboroles or boronic esters, triazine derivatives, zinc complexes, diazasilole or tetraazasilole derivatives, diazaphosphole derivatives, bridged carbazole derivatives, triphenylene derivatives, or lactams.

Suitable matrix materials for phosphorescent emitting compounds are ketones, phosphine oxides, sulfoxides and sulfones, for example according to WO 2004/013080, WO 2004/093207, WO 2006/005627 or WO 2010/006680, triarylamines, carbazole derivatives, e.g. CBP (N,N-biscarbazolylbiphenyl), m-CBP or the carbazole derivatives disclosed in WO 2005/039246, US 2005/0069729, JP 2004/288381, EP 1205527, WO 2008/086851 or US 2009/0134784, bridged carbazole derivatives, for example according to US 2009/0136779, WO 2010/050778, WO 2011/042107 or WO 2011/088877, biscarbazole derivatives, indolocarbazole derivatives, for example according to WO 2007/063754 or WO 2008/056746, indenocarbazole derivatives, for example according to WO 2010/136109 or WO 2011/000455, azacarbazoles, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, diazasilole derivatives, for example according to WO 2010/054729, diazaphosphole derivatives, for example according to WO 2010/054730, triazine derivatives, for example according to WO 2010/015306, WO 2007/063754 or WO 2008/056746, zinc complexes, for example according to EP 652273 or WO 2009/062578, dibenzofuran derivatives, for example according to WO 2009/148015, dibenzothiophene derivatives or triphenylene derivatives, or imidazo-imidazole derivatives, for example according to US 2016/0190480, US 2019/0273211 or WO 2011/160757.

Examples of suitable matrix materials are the compounds depicted below.

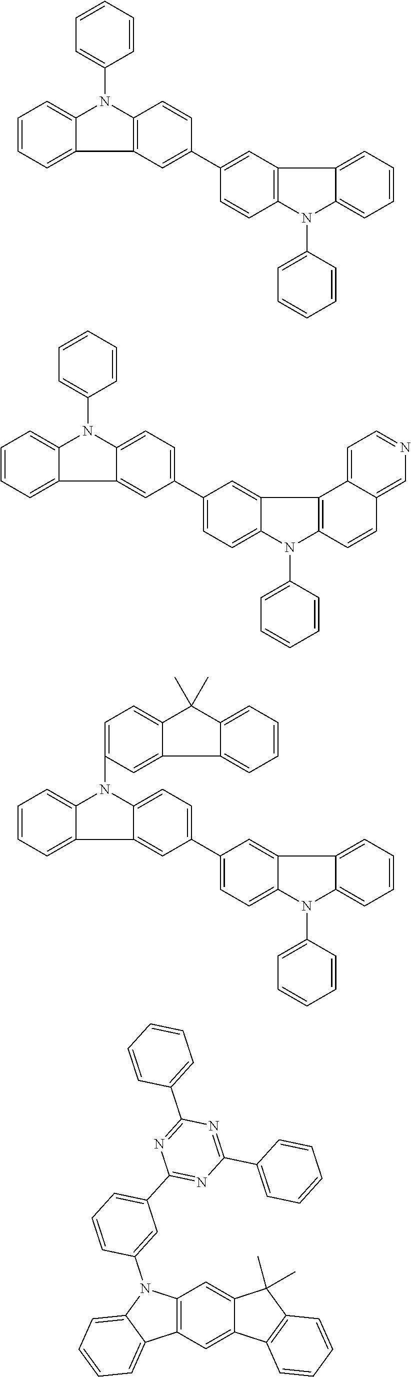 [SMILES: c1ccc(-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4c6ccncc6ccc4n5-c4ccccc4)ccc32)cc1 ; CC1(C)c2ccccc2-c2cc(-n3c4ccccc4c4cc(-c5ccc6c(c5)c5ccccc5n6-c5ccccc5)ccc43)ccc21 ; CC1(C)c2ccccc2-c2cc3c4ccccc4n(-c4cccc(-c5nc(-c6ccccc6)nc(-c6ccccc6)n5)c4)c3cc21]

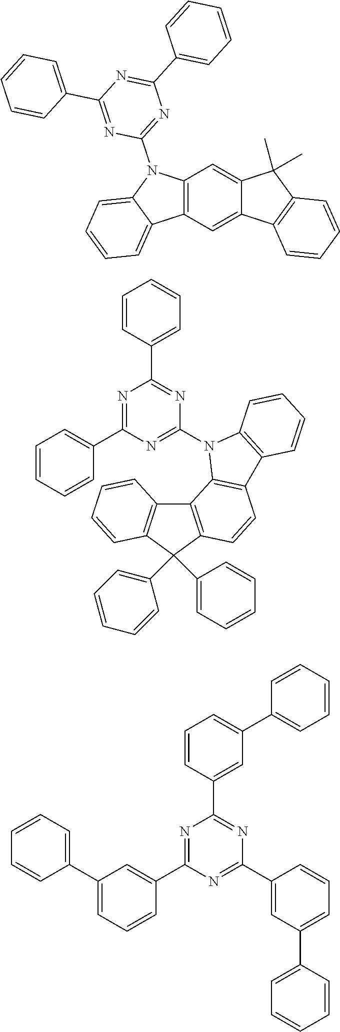

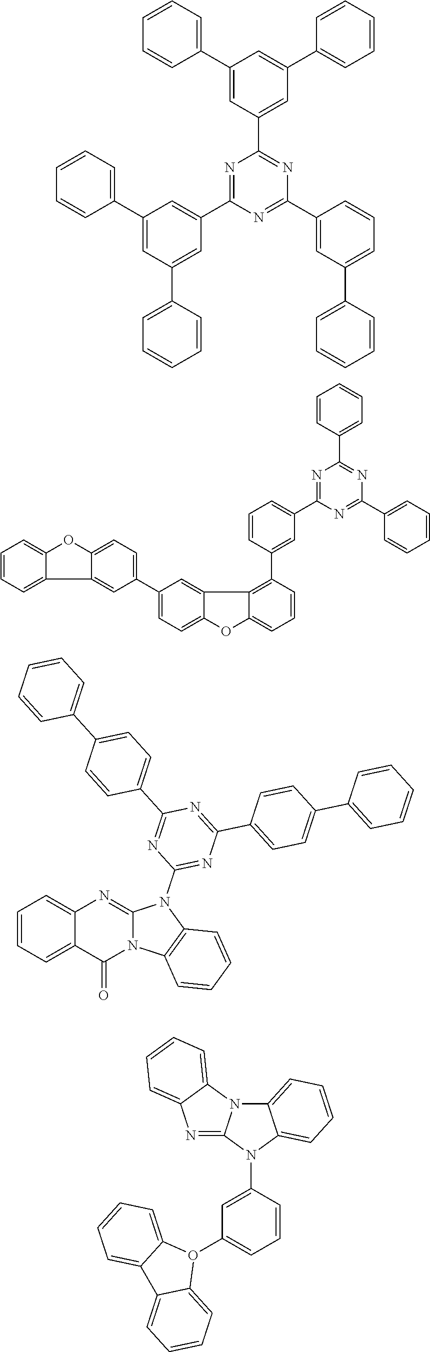

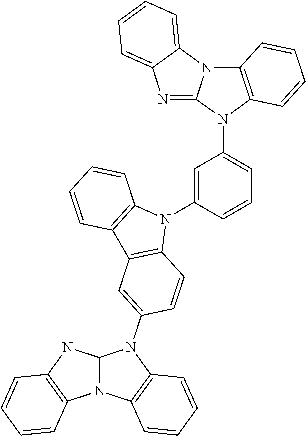 [SMILES: c1cc(-n2c3ccccc3c3cc(N4c5ccccc5N5c6ccccc6NC45)ccc32)cc(-n2c3ccccc3n3c4ccccc4nc23)c1]

Electron-Transporting Materials:

Suitable electron-transporting materials are, for example, the compounds disclosed in Y. Shirota et al., Chem. Rev. 2007, 107(4), 953-1010, or other materials used in these layers according to the prior art.

Materials used for the electron transport layer may be any materials that are used as electron transport materials in the electron transport layer according to the prior art. Especially suitable are aluminium complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives. Preferred electron-transporting compounds are shown in the following table:

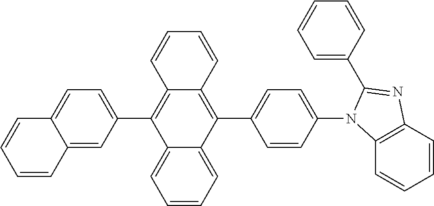 [SMILES: c1ccc(-c2nc3ccccc3n2-c2ccc(-c3c4ccccc4c(-c4ccc5ccccc5c4)c4ccccc34)cc2)cc1]

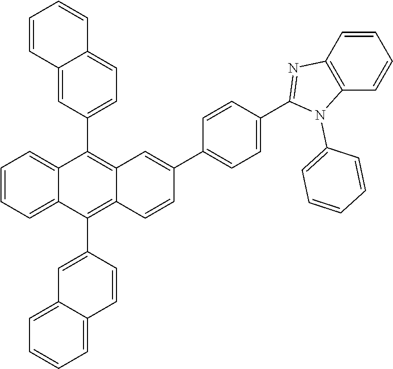 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

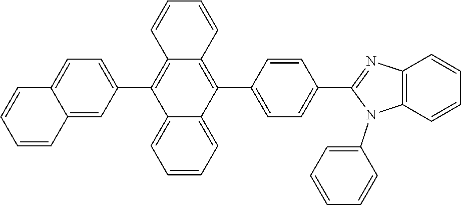 [SMILES: c1ccc(-n2c(-c3ccc(-c4c5ccccc5c(-c5ccc6ccccc6c5)c5ccccc45)cc3)nc3ccccc32)cc1]

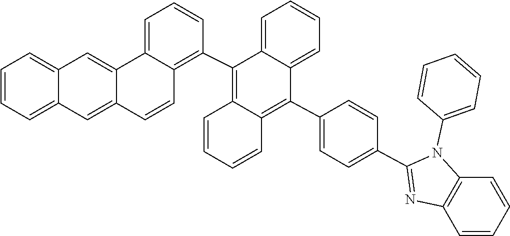 [SMILES: c1ccc(-n2c(-c3ccc(-c4c5ccccc5c(-c5cccc6c5ccc5cc7ccccc7cc56)c5ccccc45)cc3)nc3ccccc32)cc1]

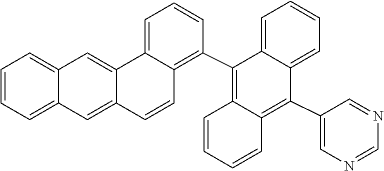 [SMILES: c1ccc2cc3c(ccc4c(-c5c6ccccc6c(-c6cncnc6)c6ccccc56)cccc43)cc2c1]

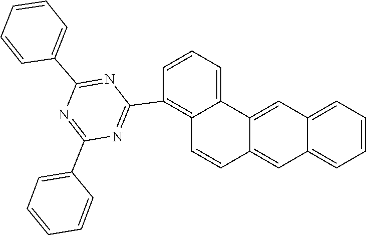 [SMILES: c1ccc(-c2nc(-c3ccccc3)nc(-c3cccc4c3ccc3cc5ccccc5cc34)n2)cc1]

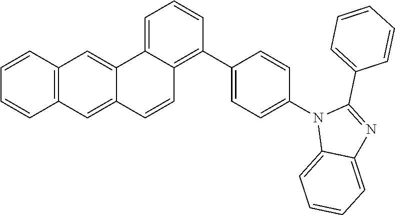 [SMILES: c1ccc(-c2nc3ccccc3n2-c2ccc(-c3cccc4c3ccc3cc5ccccc5cc34)cc2)cc1]

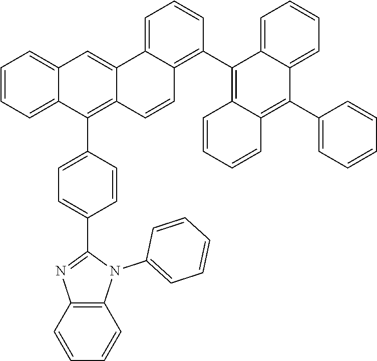 [SMILES: c1ccc(-c2c3ccccc3c(-c3cccc4c3ccc3c(-c5ccc(-c6nc7ccccc7n6-c6ccccc6)cc5)c5ccccc5cc34)c3ccccc23)cc1]

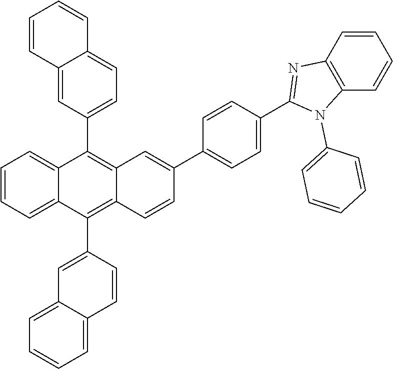 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

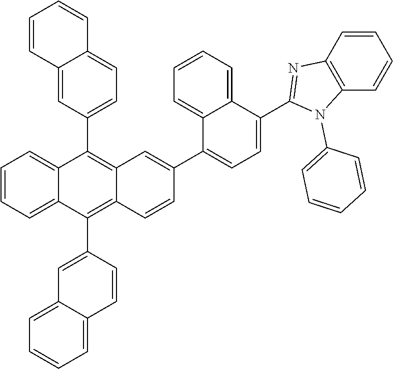 [SMILES: c1ccc(-n2c(-c3ccc(-c4ccc5c(-c6ccc7ccccc7c6)c6ccccc6c(-c6ccc7ccccc7c6)c5c4)c4ccccc34)nc3ccccc32)cc1]

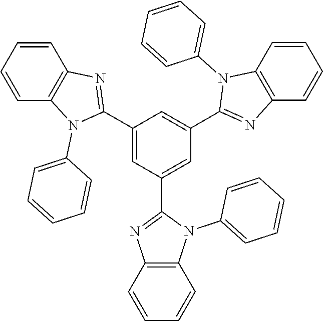 [SMILES: c1ccc(-n2c(-c3cc(-c4nc5ccccc5n4-c4ccccc4)cc(-c4nc5ccccc5n4-c4ccccc4)c3)nc3ccccc32)cc1]

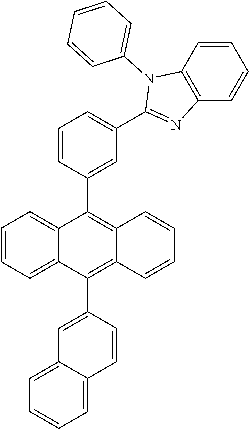 [SMILES: c1ccc(-n2c(-c3cccc(-c4c5ccccc5c(-c5ccc6ccccc6c5)c5ccccc45)c3)nc3ccccc32)cc1]

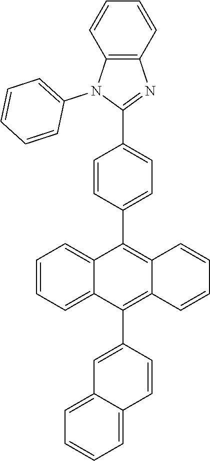 [SMILES: c1ccc(-n2c(-c3ccc(-c4c5ccccc5c(-c5ccc6ccccc6c5)c5ccccc45)cc3)nc3ccccc32)cc1]

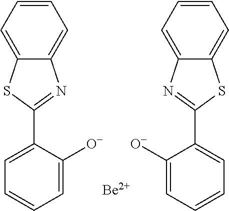

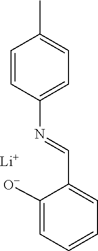 [SMILES: [Li+] ; Cc1ccc(/N=C/c2ccccc2[O-])cc1]

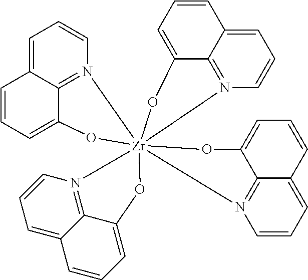

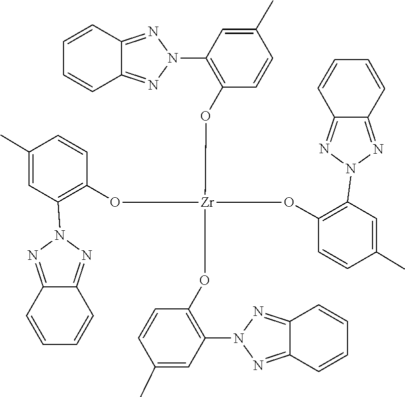

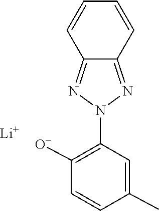 [SMILES: [Li+] ; Cc1ccc([O-])c(-n2nc3ccccc3n2)c1]

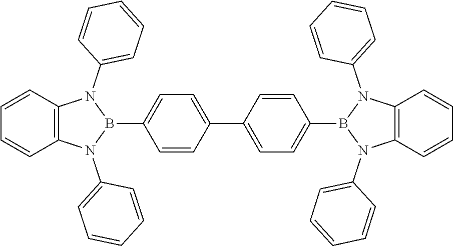 [SMILES: c1ccc(N2B(c3ccc(-c4ccc(B5N(c6ccccc6)c6ccccc6N5c5ccccc5)cc4)cc3)N(c3ccccc3)c3ccccc32)cc1]

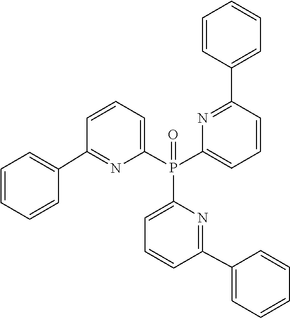 [SMILES: O=P(c1cccc(-c2ccccc2)n1)(c1cccc(-c2ccccc2)n1)c1cccc(-c2ccccc2)n1]

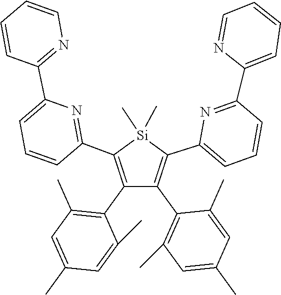 [SMILES: Cc1cc(C)c(C2=C(c3cccc(-c4ccccn4)n3)[Si](C)(C)C(c3cccc(-c4ccccn4)n3)=C2c2c(C)cc(C)cc2C)c(C)c1]

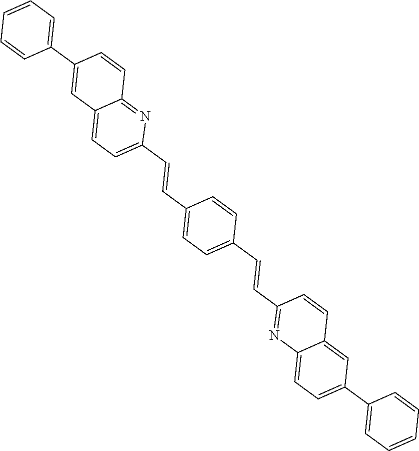 [SMILES: C(=C\c1ccc2cc(-c3ccccc3)ccc2n1)/c1ccc(/C=C/c2ccc3cc(-c4ccccc4)ccc3n2)cc1]

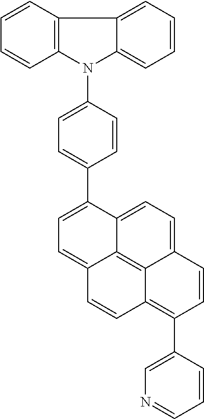 [SMILES: c1cncc(-c2ccc3ccc4c(-c5ccc(-n6c7ccccc7c7ccccc76)cc5)ccc5ccc2c3c54)c1]

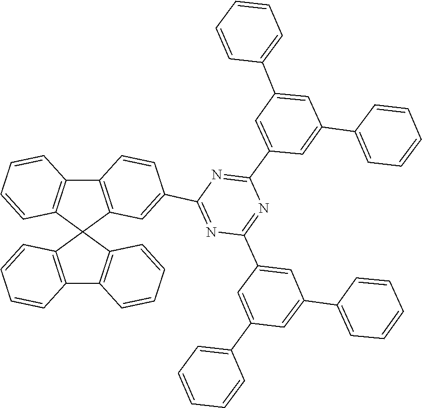 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

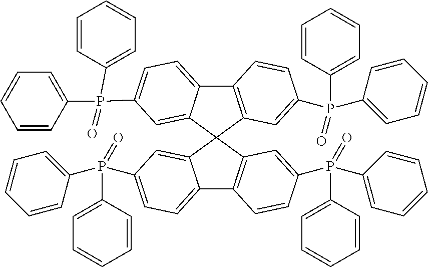 [SMILES: O=P(c1ccccc1)(c1ccccc1)c1ccc2c(c1)C1(c3cc(P(=O)(c4ccccc4)c4ccccc4)ccc3-2)c2cc(P(=O)(c3ccccc3)c3ccccc3)ccc2-c2ccc(P(=O)(c3ccccc3)c3ccccc3)cc21]

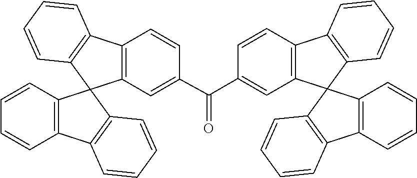 [SMILES: O=C(c1ccc2c(c1)C1(c3ccccc3-c3ccccc31)c1ccccc1-2)c1ccc2c(c1)C1(c3ccccc3-c3ccccc31)c1ccccc1-2]

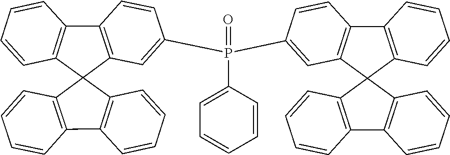 [SMILES: O=P(c1ccccc1)(c1ccc2c(c1)C1(c3ccccc3-c3ccccc31)c1ccccc1-2)c1ccc2c(c1)C1(c3ccccc3-c3ccccc31)c1ccccc1-2]

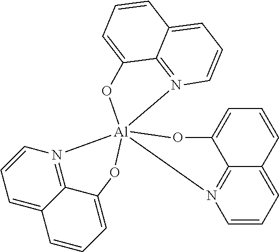

Hole-Transporting Materials:

Further compounds which, in addition to the compounds of the formula (1), are used with preference in hole-transporting layers of the OLEDs of the invention are indenofluoreneamine derivatives, amine derivatives, hexaazatriphenylene derivatives, amine derivatives with fused aromatic systems, monobenzoindenofluoreneamines, dibenzoindenofluoreneamines, spirobifluoreneamines, fluoreneamines, spirodibenzopyranamines, dihydroacridine derivatives, spirodibenzofurans and spirodibenzothiophenes, phenanthrenediarylamines, spirotribenzotropolones, spirobifluorenes having meta-phenyldiamine groups, spirobisacridines, xanthenediarylamines, and 9,10-dihydroanthracene spiro compounds having diarylamino groups. Preferred hole-transporting compounds are shown in the following table:

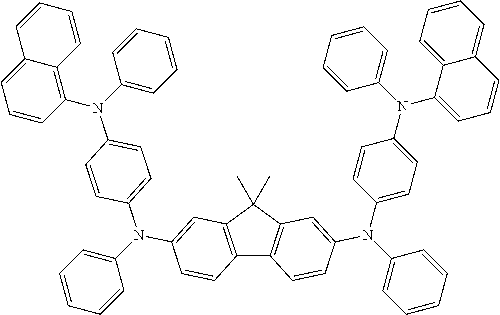 [SMILES: CC1(C)c2cc(N(c3ccccc3)c3ccc(N(c4ccccc4)c4cccc5ccccc45)cc3)ccc2-c2ccc(N(c3ccccc3)c3ccc(N(c4ccccc4)c4cccc5ccccc45)cc3)cc21]

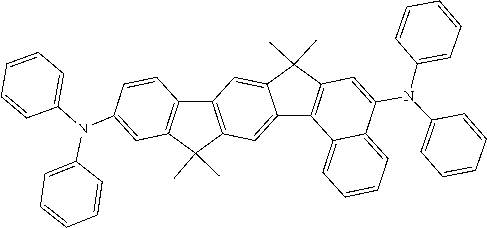 [SMILES: CC1(C)c2cc(N(c3ccccc3)c3ccccc3)ccc2-c2cc3c(cc21)-c1c(cc(N(c2ccccc2)c2ccccc2)c2ccccc12)C3(C)C]

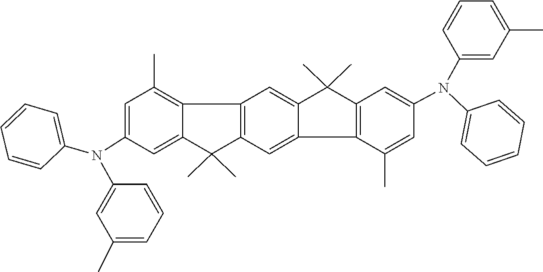 [SMILES: Cc1cccc(N(c2ccccc2)c2cc(C)c3c(c2)C(C)(C)c2cc4c(cc2-3)C(C)(C)c2cc(N(c3ccccc3)c3cccc(C)c3)cc(C)c2-4)c1]

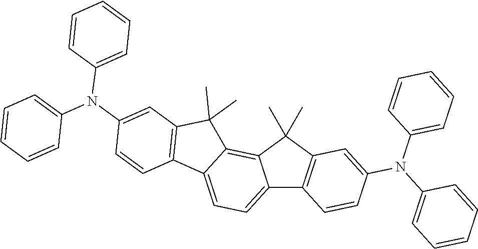 [SMILES: CC1(C)c2cc(N(c3ccccc3)c3ccccc3)ccc2-c2ccc3c(c21)C(C)(C)c1cc(N(c2ccccc2)c2ccccc2)ccc1-3]

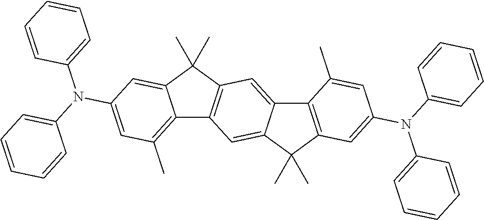 [SMILES: Cc1cc(N(c2ccccc2)c2ccccc2)cc2c1-c1cc3c(cc1C2(C)C)-c1c(C)cc(N(c2ccccc2)c2ccccc2)cc1C3(C)C]

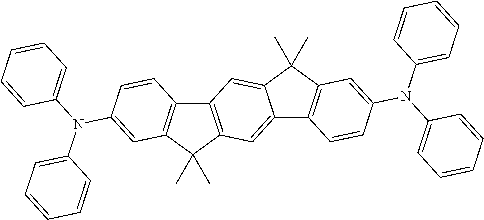 [SMILES: CC1(C)c2cc(N(c3ccccc3)c3ccccc3)ccc2-c2cc3c(cc21)-c1ccc(N(c2ccccc2)c2ccccc2)cc1C3(C)C]

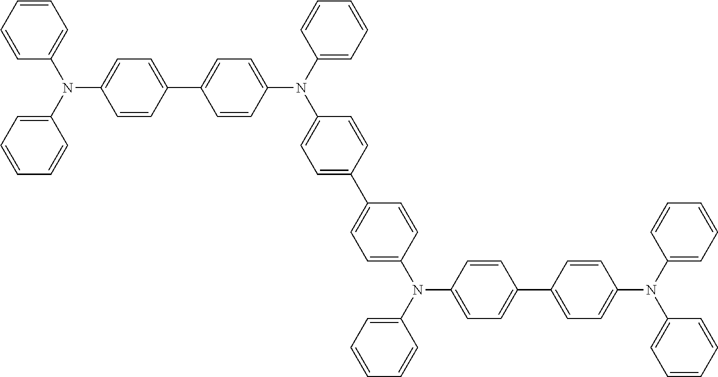 [SMILES: c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1]

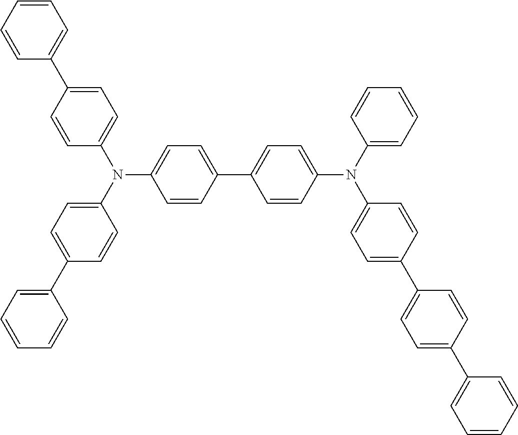 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

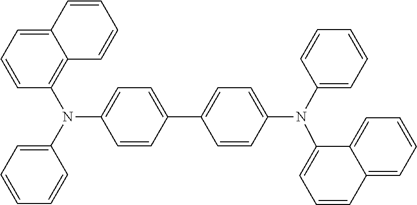 [SMILES: c1ccc(N(c2ccc(-c3ccc(N(c4ccccc4)c4cccc5ccccc45)cc3)cc2)c2cccc3ccccc23)cc1]

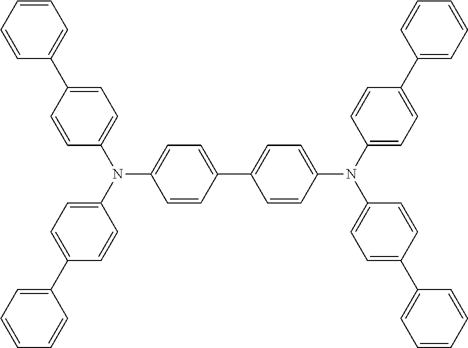 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

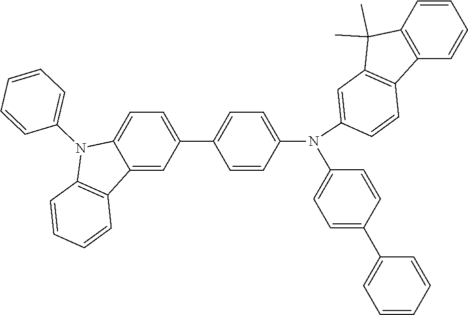 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

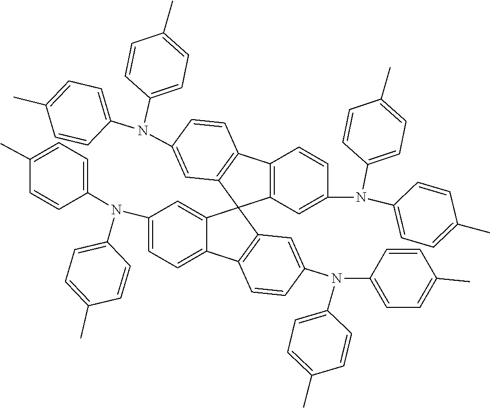

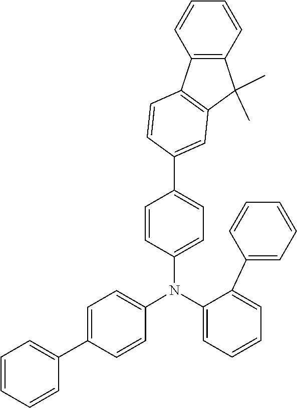 [SMILES: CC1(C)c2ccccc2-c2ccc(-c3ccc(N(c4ccc(-c5ccccc5)cc4)c4ccccc4-c4ccccc4)cc3)cc21]

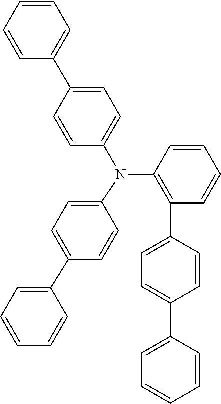 [SMILES: c1ccc(-c2ccc(-c3ccccc3N(c3ccc(-c4ccccc4)cc3)c3ccc(-c4ccccc4)cc3)cc2)cc1]

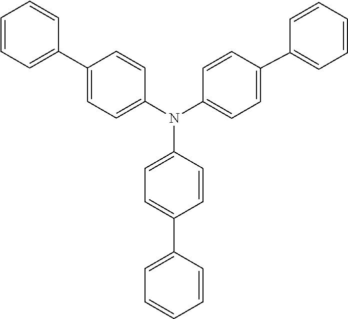 [SMILES: c1ccc(-c2ccc(N(c3ccc(-c4ccccc4)cc3)c3ccc(-c4ccccc4)cc3)cc2)cc1]

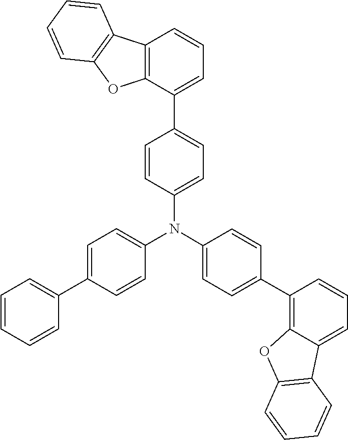 [SMILES: c1ccc(-c2ccc(N(c3ccc(-c4cccc5c4oc4ccccc45)cc3)c3ccc(-c4cccc5c4oc4ccccc45)cc3)cc2)cc1]

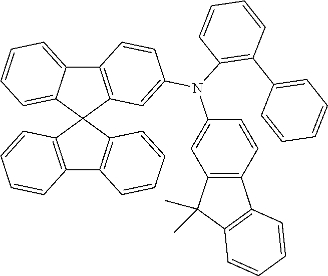 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

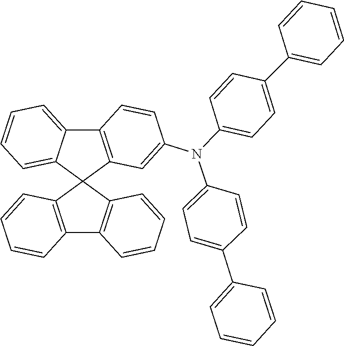 [SMILES: c1ccc(-c2ccc(N(c3ccc(-c4ccccc4)cc3)c3ccc4c(c3)C3(c5ccccc5-c5ccccc53)c3ccccc3-4)cc2)cc1]

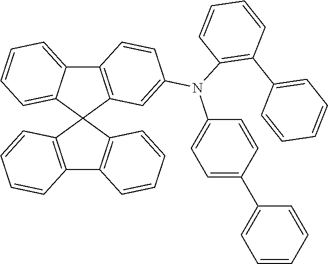 [SMILES: c1ccc(-c2ccc(N(c3ccc4c(c3)C3(c5ccccc5-c5ccccc53)c3ccccc3-4)c3ccccc3-c3ccccc3)cc2)cc1]

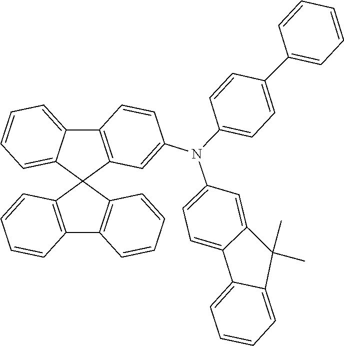 [SMILES: CC1(C)c2ccccc2-c2ccc(N(c3ccc(-c4ccccc4)cc3)c3ccc4c(c3)C3(c5ccccc5-c5ccccc53)c3ccccc3-4)cc21]

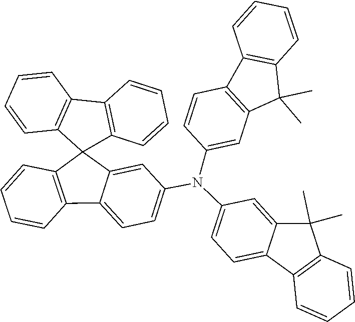 [SMILES: CC1(C)c2ccccc2-c2ccc(N(c3ccc4c(c3)C(C)(C)c3ccccc3-4)c3ccc4c(c3)C3(c5ccccc5-c5ccccc53)c3ccccc3-4)cc21]

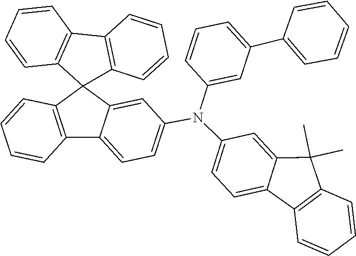 [SMILES: CC1(C)c2ccccc2-c2ccc(N(c3cccc(-c4ccccc4)c3)c3ccc4c(c3)C3(c5ccccc5-c5ccccc53)c3ccccc3-4)cc21]

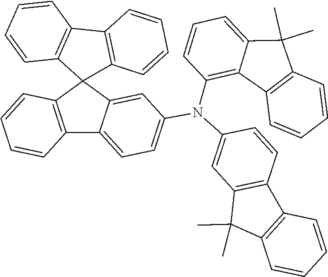 [SMILES: CC1(C)c2ccccc2-c2ccc(N(c3ccc4c(c3)C3(c5ccccc5-c5ccccc53)c3ccccc3-4)c3cccc4c3-c3ccccc3C4(C)C)cc21]

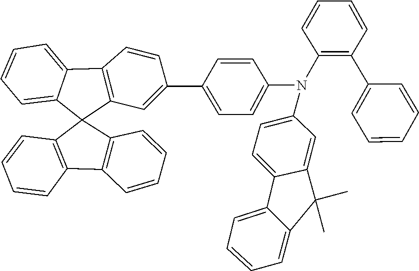 [SMILES: CC1(C)c2ccccc2-c2ccc(N(c3ccc(-c4ccc5c(c4)C4(c6ccccc6-c6ccccc64)c4ccccc4-5)cc3)c3ccccc3-c3ccccc3)cc21]

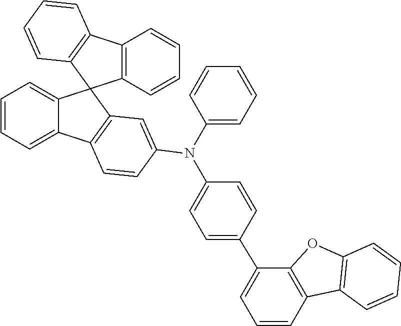 [SMILES: c1ccc(N(c2ccc(-c3cccc4c3oc3ccccc34)cc2)c2ccc3c(c2)C2(c4ccccc4-c4ccccc42)c2ccccc2-3)cc1]

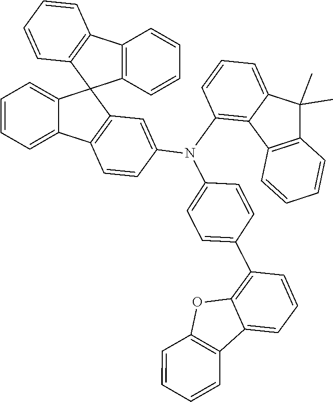 [SMILES: CC1(C)c2ccccc2-c2c(N(c3ccc(-c4cccc5c4oc4ccccc45)cc3)c3ccc4c(c3)C3(c5ccccc5-c5ccccc53)c3ccccc3-4)cccc21]

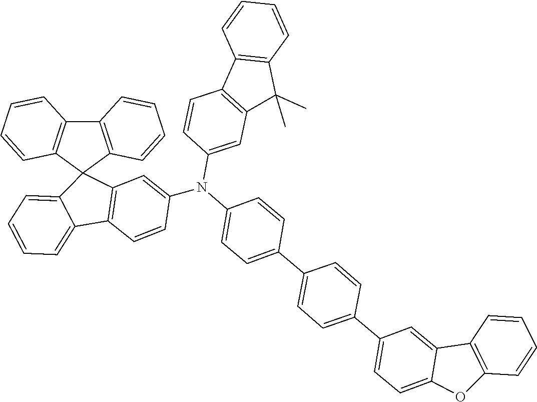 [SMILES: CC1(C)c2ccccc2-c2ccc(N(c3ccc(-c4ccc(-c5ccc6oc7ccccc7c6c5)cc4)cc3)c3ccc4c(c3)C3(c5ccccc5-c5ccccc53)c3ccccc3-4)cc21]

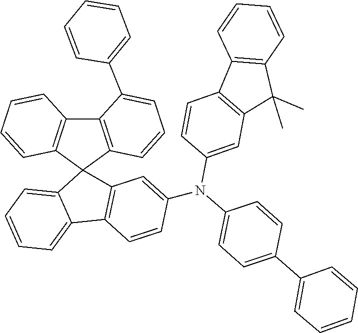 [SMILES: CC1(C)c2ccccc2-c2ccc(N(c3ccc(-c4ccccc4)cc3)c3ccc4c(c3)C3(c5ccccc5-4)c4ccccc4-c4c(-c5ccccc5)cccc43)cc21]

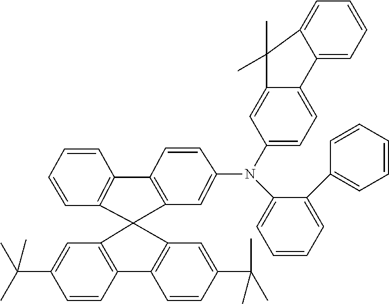 [SMILES: CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2]

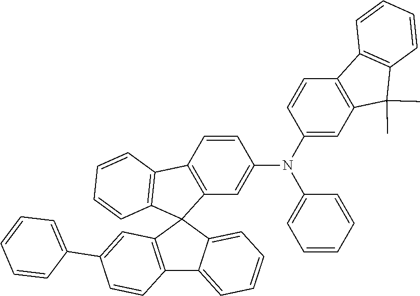 [SMILES: CC1(C)c2ccccc2-c2ccc(N(c3ccccc3)c3ccc4c(c3)C3(c5ccccc5-c5ccc(-c6ccccc6)cc53)c3ccccc3-4)cc21]

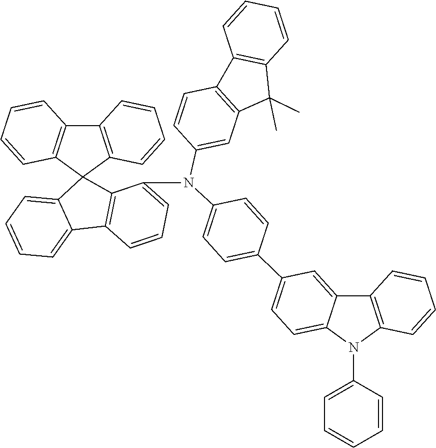 [SMILES: CC1(C)c2ccccc2-c2ccc(N(c3ccc(-c4ccc5c(c4)c4ccccc4n5-c4ccccc4)cc3)c3cccc4c3C3(c5ccccc5-c5ccccc53)c3ccccc3-4)cc21]

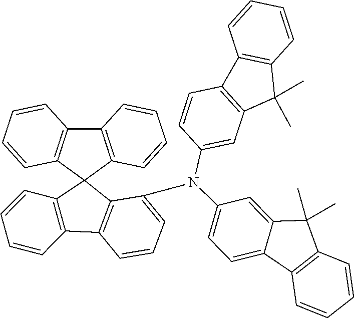 [SMILES: CC1(C)c2ccccc2-c2ccc(N(c3ccc4c(c3)C(C)(C)c3ccccc3-4)c3cccc4c3C3(c5ccccc5-c5ccccc53)c3ccccc3-4)cc21]

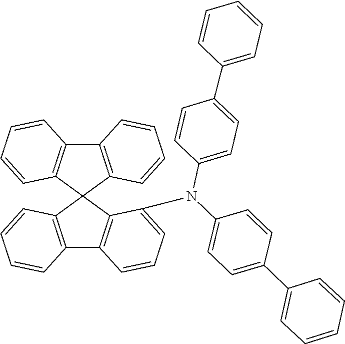 [SMILES: c1ccc(-c2ccc(N(c3ccc(-c4ccccc4)cc3)c3cccc4c3C3(c5ccccc5-c5ccccc53)c3ccccc3-4)cc2)cc1]

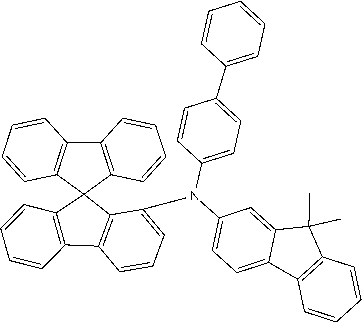 [SMILES: CC1(C)c2ccccc2-c2ccc(N(c3ccc(-c4ccccc4)cc3)c3cccc4c3C3(c5ccccc5-c5ccccc53)c3ccccc3-4)cc21]

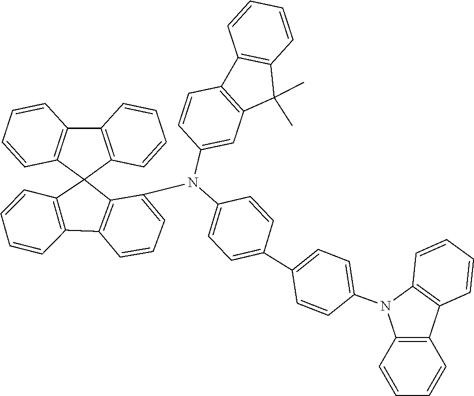 [SMILES: CC1(C)c2ccccc2-c2ccc(N(c3ccc(-c4ccc(-n5c6ccccc6c6ccccc65)cc4)cc3)c3cccc4c3C3(c5ccccc5-c5ccccc53)c3ccccc3-4)cc21]

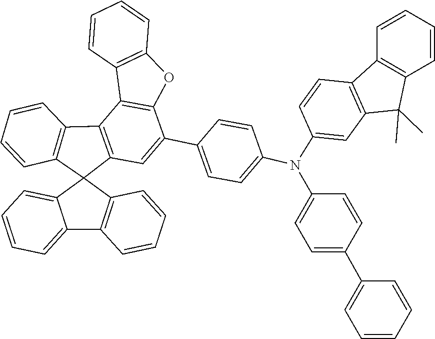 [SMILES: CC1(C)c2ccccc2-c2ccc(N(c3ccc(-c4ccccc4)cc3)c3ccc(-c4cc5c(c6c4oc4ccccc46)-c4ccccc4C54c5ccccc5-c5ccccc54)cc3)cc21]

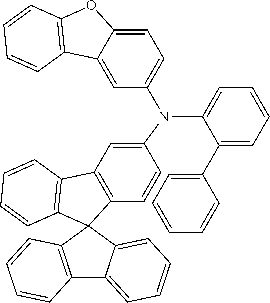 [SMILES: c1ccc(-c2ccccc2N(c2ccc3c(c2)-c2ccccc2C32c3ccccc3-c3ccccc32)c2ccc3oc4ccccc4c3c2)cc1]

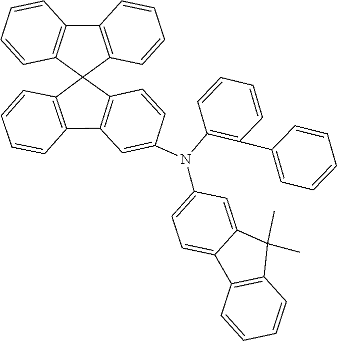 [SMILES: CC1(C)c2ccccc2-c2ccc(N(c3ccc4c(c3)-c3ccccc3C43c4ccccc4-c4ccccc43)c3ccccc3-c3ccccc3)cc21]

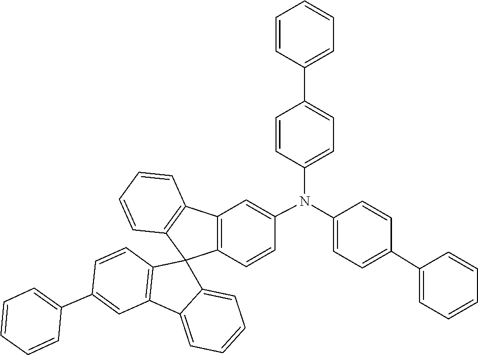 [SMILES: c1ccc(-c2ccc(N(c3ccc(-c4ccccc4)cc3)c3ccc4c(c3)-c3ccccc3C43c4ccccc4-c4cc(-c5ccccc5)ccc43)cc2)cc1]

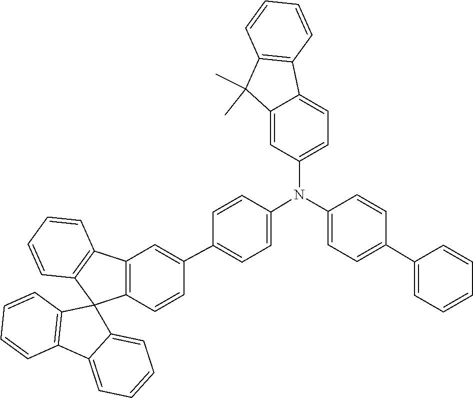 [SMILES: CC1(C)c2ccccc2-c2ccc(N(c3ccc(-c4ccccc4)cc3)c3ccc(-c4ccc5c(c4)-c4ccccc4C54c5ccccc5-c5ccccc54)cc3)cc21]

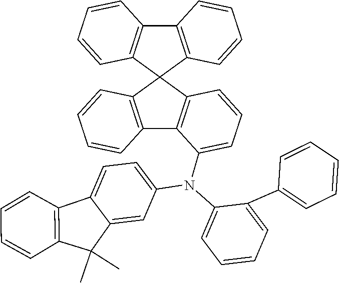 [SMILES: CC1(C)c2ccccc2-c2ccc(N(c3ccccc3-c3ccccc3)c3cccc4c3-c3ccccc3C43c4ccccc4-c4ccccc43)cc21]

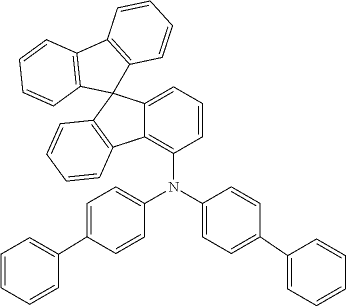 [SMILES: c1ccc(-c2ccc(N(c3ccc(-c4ccccc4)cc3)c3cccc4c3-c3ccccc3C43c4ccccc4-c4ccccc43)cc2)cc1]

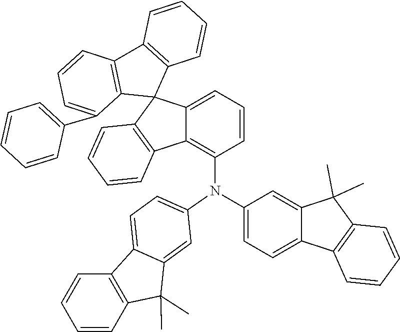 [SMILES: CC1(C)c2ccccc2-c2ccc(N(c3ccc4c(c3)C(C)(C)c3ccccc3-4)c3cccc4c3-c3ccccc3C43c4ccccc4-c4cccc(-c5ccccc5)c43)cc21]

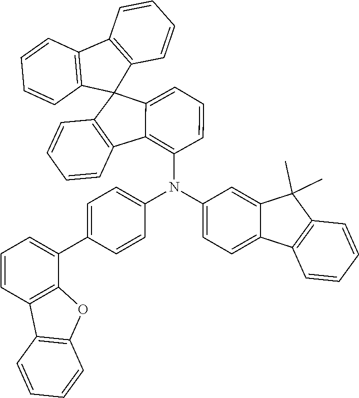 [SMILES: CC1(C)c2ccccc2-c2ccc(N(c3ccc(-c4cccc5c4oc4ccccc45)cc3)c3cccc4c3-c3ccccc3C43c4ccccc4-c4ccccc43)cc21]

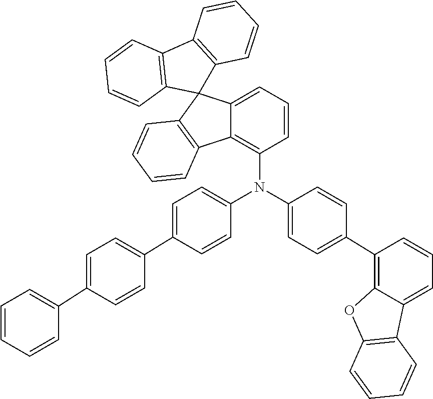 [SMILES: c1ccc(-c2ccc(-c3ccc(N(c4ccc(-c5cccc6c5oc5ccccc56)cc4)c4cccc5c4-c4ccccc4C54c5ccccc5-c5ccccc54)cc3)cc2)cc1]

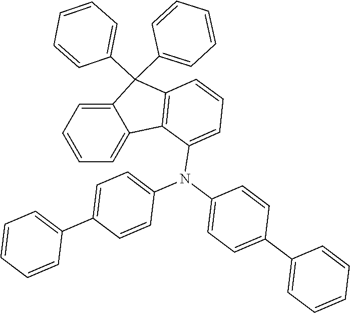 [SMILES: c1ccc(-c2ccc(N(c3ccc(-c4ccccc4)cc3)c3cccc4c3-c3ccccc3C4(c3ccccc3)c3ccccc3)cc2)cc1]

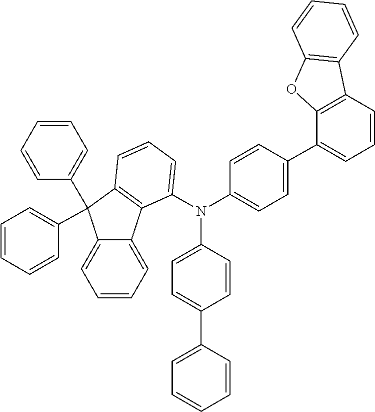 [SMILES: c1ccc(-c2ccc(N(c3ccc(-c4cccc5c4oc4ccccc45)cc3)c3cccc4c3-c3ccccc3C4(c3ccccc3)c3ccccc3)cc2)cc1]

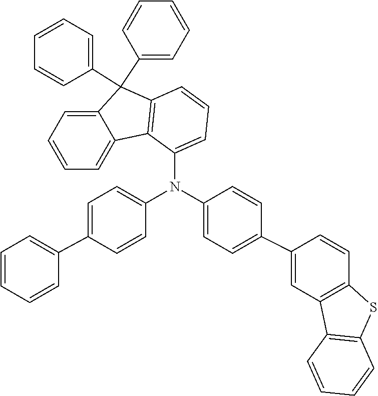 [SMILES: c1ccc(-c2ccc(N(c3ccc(-c4ccc5sc6ccccc6c5c4)cc3)c3cccc4c3-c3ccccc3C4(c3ccccc3)c3ccccc3)cc2)cc1]

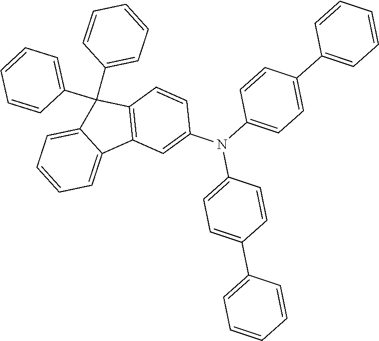 [SMILES: c1ccc(-c2ccc(N(c3ccc(-c4ccccc4)cc3)c3ccc4c(c3)-c3ccccc3C4(c3ccccc3)c3ccccc3)cc2)cc1]

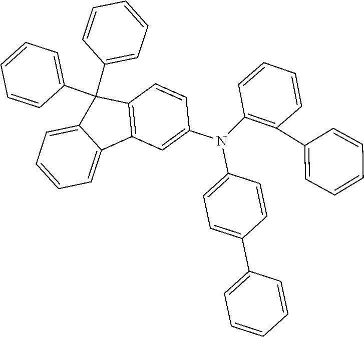 [SMILES: c1ccc(-c2ccc(N(c3ccc4c(c3)-c3ccccc3C4(c3ccccc3)c3ccccc3)c3ccccc3-c3ccccc3)cc2)cc1]

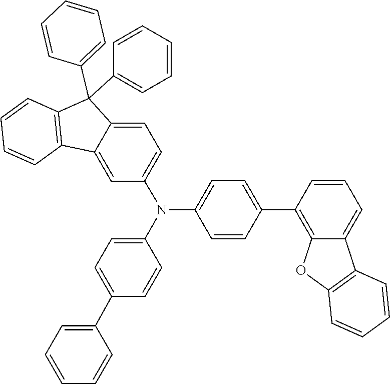 [SMILES: c1ccc(-c2ccc(N(c3ccc(-c4cccc5c4oc4ccccc45)cc3)c3ccc4c(c3)-c3ccccc3C4(c3ccccc3)c3ccccc3)cc2)cc1]

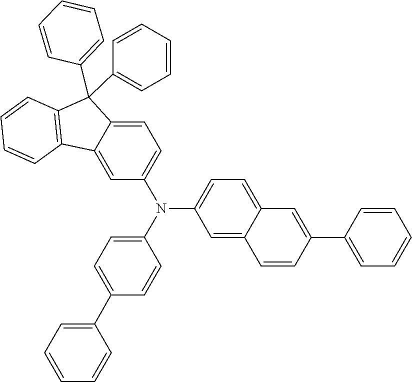 [SMILES: c1ccc(-c2ccc(N(c3ccc4c(c3)-c3ccccc3C4(c3ccccc3)c3ccccc3)c3ccc4cc(-c5ccccc5)ccc4c3)cc2)cc1]

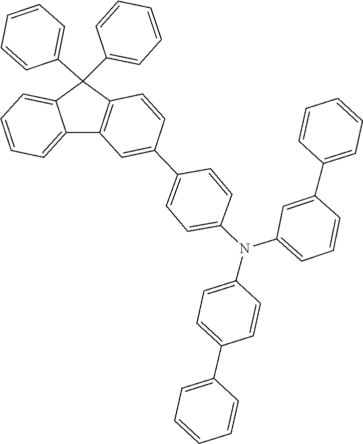 [SMILES: c1ccc(-c2ccc(N(c3ccc(-c4ccc5c(c4)-c4ccccc4C5(c4ccccc4)c4ccccc4)cc3)c3cccc(-c4ccccc4)c3)cc2)cc1]

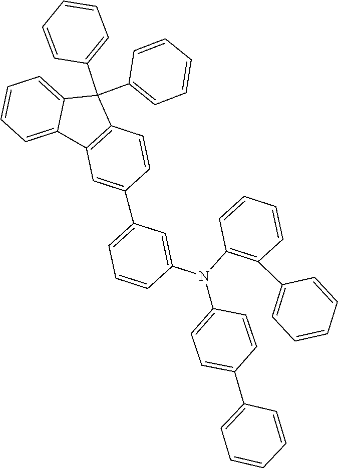 [SMILES: c1ccc(-c2ccc(N(c3cccc(-c4ccc5c(c4)-c4ccccc4C5(c4ccccc4)c4ccccc4)c3)c3ccccc3-c3ccccc3)cc2)cc1]

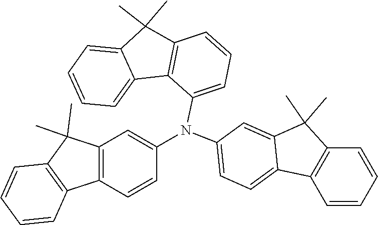 [SMILES: CC1(C)c2ccccc2-c2ccc(N(c3ccc4c(c3)C(C)(C)c3ccccc3-4)c3cccc4c3-c3ccccc3C4(C)C)cc21]

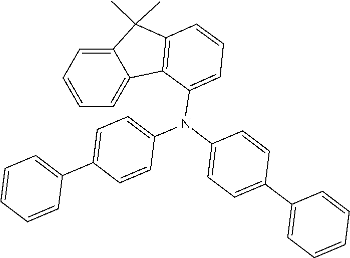 [SMILES: CC1(C)c2ccccc2-c2c(N(c3ccc(-c4ccccc4)cc3)c3ccc(-c4ccccc4)cc3)cccc21]

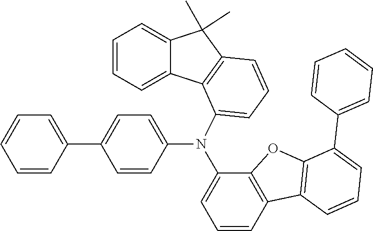 [SMILES: CC1(C)c2ccccc2-c2c(N(c3ccc(-c4ccccc4)cc3)c3cccc4c3oc3c(-c5ccccc5)cccc34)cccc21]

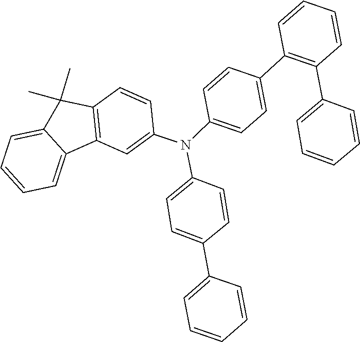 [SMILES: CC1(C)c2ccccc2-c2cc(N(c3ccc(-c4ccccc4)cc3)c3ccc(-c4ccccc4-c4ccccc4)cc3)ccc21]

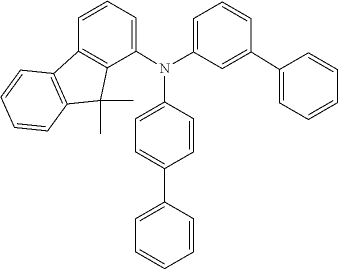 [SMILES: CC1(C)c2ccccc2-c2cccc(N(c3ccc(-c4ccccc4)cc3)c3cccc(-c4ccccc4)c3)c21]

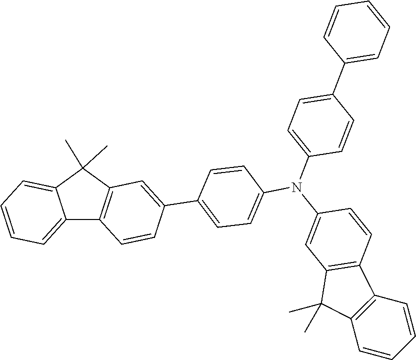 [SMILES: CC1(C)c2ccccc2-c2ccc(-c3ccc(N(c4ccc(-c5ccccc5)cc4)c4ccc5c(c4)C(C)(C)c4ccccc4-5)cc3)cc21]

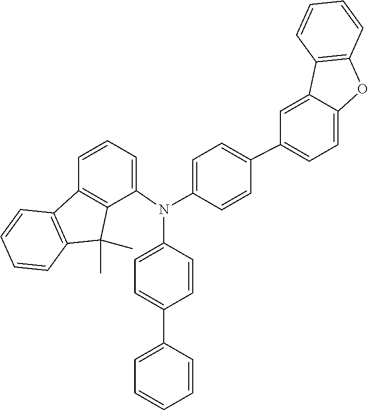 [SMILES: CC1(C)c2ccccc2-c2cccc(N(c3ccc(-c4ccccc4)cc3)c3ccc(-c4ccc5oc6ccccc6c5c4)cc3)c21]

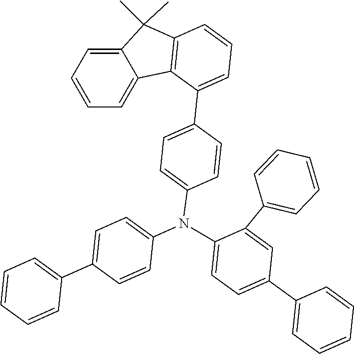 [SMILES: CC1(C)c2ccccc2-c2c(-c3ccc(N(c4ccc(-c5ccccc5)cc4)c4ccc(-c5ccccc5)cc4-c4ccccc4)cc3)cccc21]

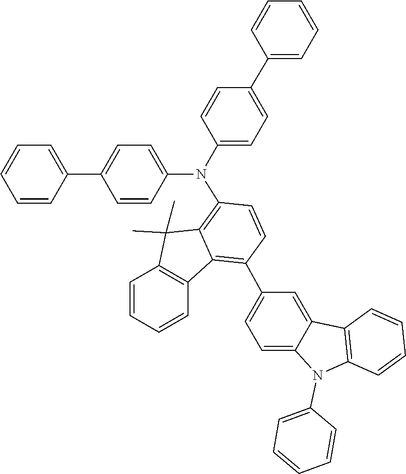 [SMILES: CC1(C)c2ccccc2-c2c(-c3ccc4c(c3)c3ccccc3n4-c3ccccc3)ccc(N(c3ccc(-c4ccccc4)cc3)c3ccc(-c4ccccc4)cc3)c21]

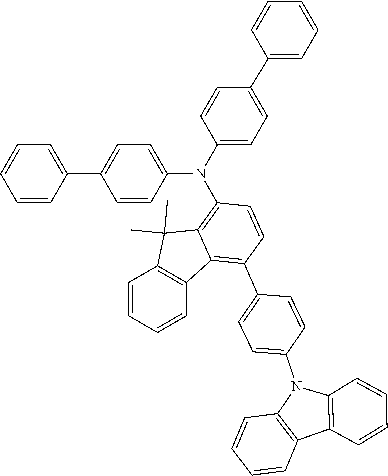 [SMILES: CC1(C)c2ccccc2-c2c(-c3ccc(-n4c5ccccc5c5ccccc54)cc3)ccc(N(c3ccc(-c4ccccc4)cc3)c3ccc(-c4ccccc4)cc3)c21]

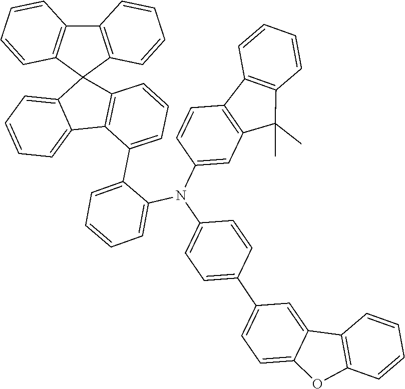 [SMILES: CC1(C)c2ccccc2-c2ccc(N(c3ccc(-c4ccc5oc6ccccc6c5c4)cc3)c3ccccc3-c3cccc4c3-c3ccccc3C43c4ccccc4-c4ccccc43)cc21]

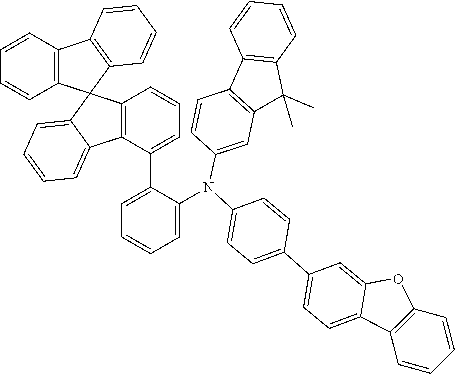 [SMILES: CC1(C)c2ccccc2-c2ccc(N(c3ccc(-c4ccc5c(c4)oc4ccccc45)cc3)c3ccccc3-c3cccc4c3-c3ccccc3C43c4ccccc4-c4ccccc43)cc21]

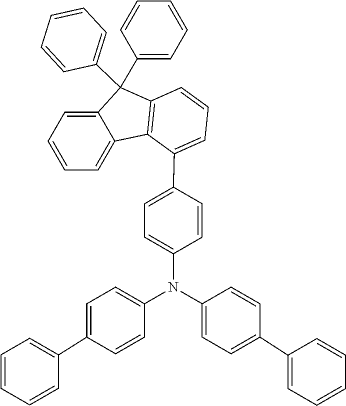 [SMILES: c1ccc(-c2ccc(N(c3ccc(-c4ccccc4)cc3)c3ccc(-c4cccc5c4-c4ccccc4C5(c4ccccc4)c4ccccc4)cc3)cc2)cc1]

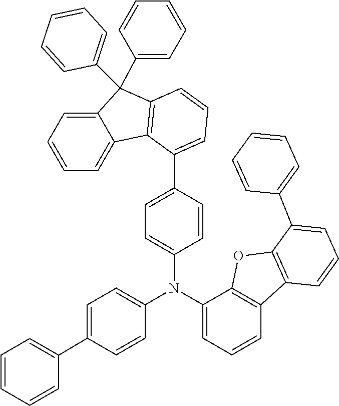 [SMILES: c1ccc(-c2ccc(N(c3ccc(-c4cccc5c4-c4ccccc4C5(c4ccccc4)c4ccccc4)cc3)c3cccc4c3oc3c(-c5ccccc5)cccc34)cc2)cc1]

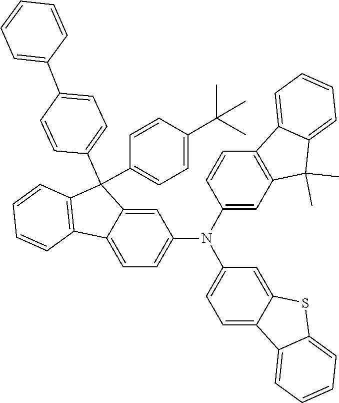 [SMILES: CC(C)(C)c1ccc(C2(c3ccc(-c4ccccc4)cc3)c3ccccc3-c3ccc(N(c4ccc5c(c4)C(C)(C)c4ccccc4-5)c4ccc5c(c4)sc4ccccc45)cc32)cc1]

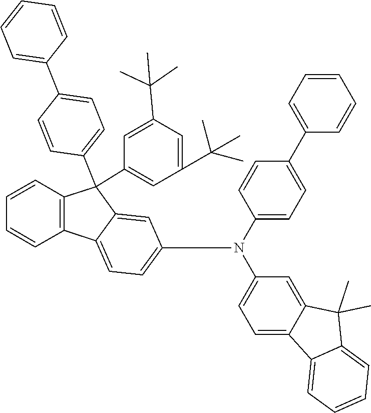 [SMILES: CC(C)(C)c1cc(C(C)(C)C)cc(C2(c3ccc(-c4ccccc4)cc3)c3ccccc3-c3ccc(N(c4ccc(-c5ccccc5)cc4)c4ccc5c(c4)C(C)(C)c4ccccc4-5)cc32)c1]

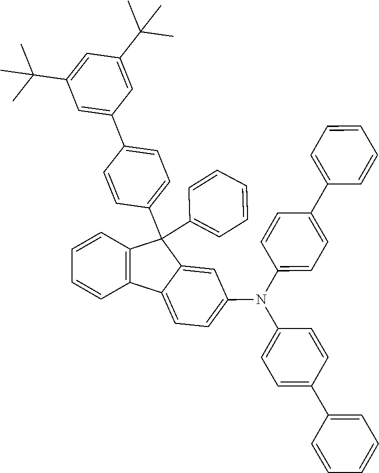 [SMILES: CC(C)(C)c1cc(-c2ccc(C3(c4ccccc4)c4ccccc4-c4ccc(N(c5ccc(-c6ccccc6)cc5)c5ccc(-c6ccccc6)cc5)cc43)cc2)cc(C(C)(C)C)c1]

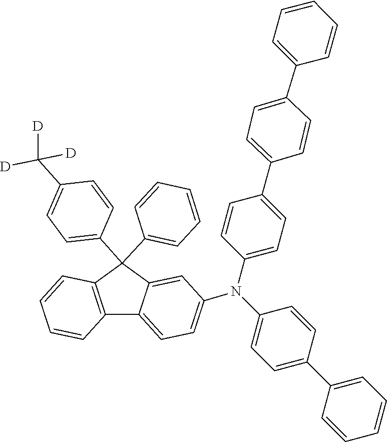 [SMILES: [2H]C([2H])([2H])c1ccc(C2(c3ccccc3)c3ccccc3-c3ccc(N(c4ccc(-c5ccccc5)cc4)c4ccc(-c5ccc(-c6ccccc6)cc5)cc4)cc32)cc1]

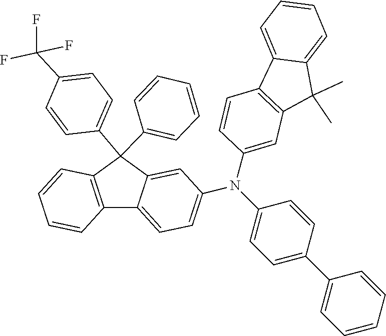 [SMILES: CC1(C)c2ccccc2-c2ccc(N(c3ccc(-c4ccccc4)cc3)c3ccc4c(c3)C(c3ccccc3)(c3ccc(C(F)(F)F)cc3)c3ccccc3-4)cc21]

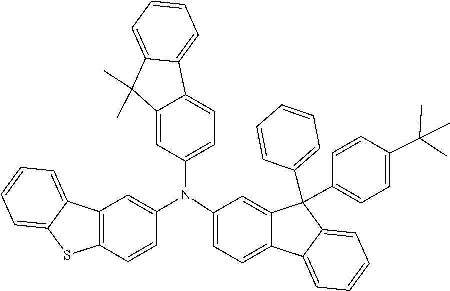 [SMILES: CC(C)(C)c1ccc(C2(c3ccccc3)c3ccccc3-c3ccc(N(c4ccc5c(c4)C(C)(C)c4ccccc4-5)c4ccc5sc6ccccc6c5c4)cc32)cc1]

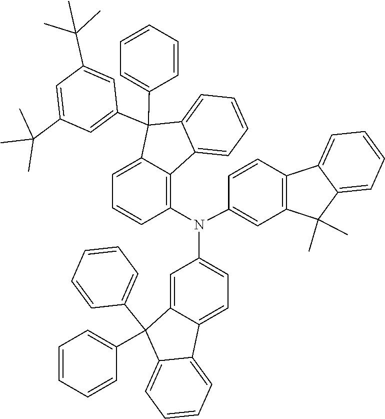 [SMILES: CC(C)(C)c1cc(C(C)(C)C)cc(C2(c3ccccc3)c3ccccc3-c3c(N(c4ccc5c(c4)C(C)(C)c4ccccc4-5)c4ccc5c(c4)C(c4ccccc4)(c4ccccc4)c4ccccc4-5)cccc32)c1]

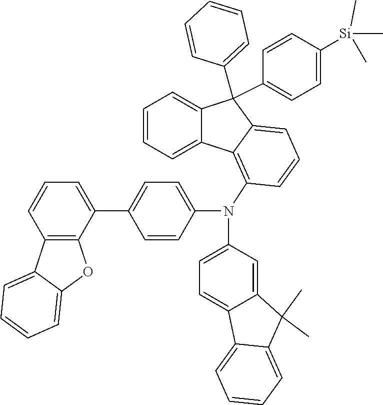 [SMILES: CC1(C)c2ccccc2-c2ccc(N(c3ccc(-c4cccc5c4oc4ccccc45)cc3)c3cccc4c3-c3ccccc3C4(c3ccccc3)c3ccc([Si](C)(C)C)cc3)cc21]

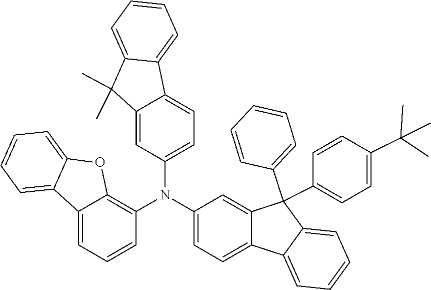 [SMILES: CC(C)(C)c1ccc(C2(c3ccccc3)c3ccccc3-c3ccc(N(c4ccc5c(c4)C(C)(C)c4ccccc4-5)c4cccc5c4oc4ccccc45)cc32)cc1]

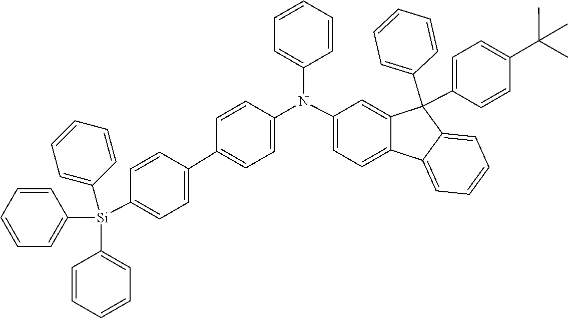 [SMILES: CC(C)(C)c1ccc(C2(c3ccccc3)c3ccccc3-c3ccc(N(c4ccccc4)c4ccc(-c5ccc([Si](c6ccccc6)(c6ccccc6)c6ccccc6)cc5)cc4)cc32)cc1]

The following compounds HT-1 to HT-68 are suitable for use in a layer having a hole-transporting function, especially in a hole injection layer, a hole transport layer and/or an electron blocker layer, or for use in an emitting layer as matrix material, especially as matrix material in an emitting layer comprising one or more phosphorescent emitters: Most preferred is the use in a hole injection layer, hole transport layer and/or electron blocker layer. The compounds may be used either in combination with a compound of the invention in one layer or in a separate layer, since the compounds themselves already have very good hole injection properties and hole-transporting and electron-blocking properties, and can considerably improve the performance data (e.g. efficiency, lifetime and voltage) of organic electroluminescent devices.

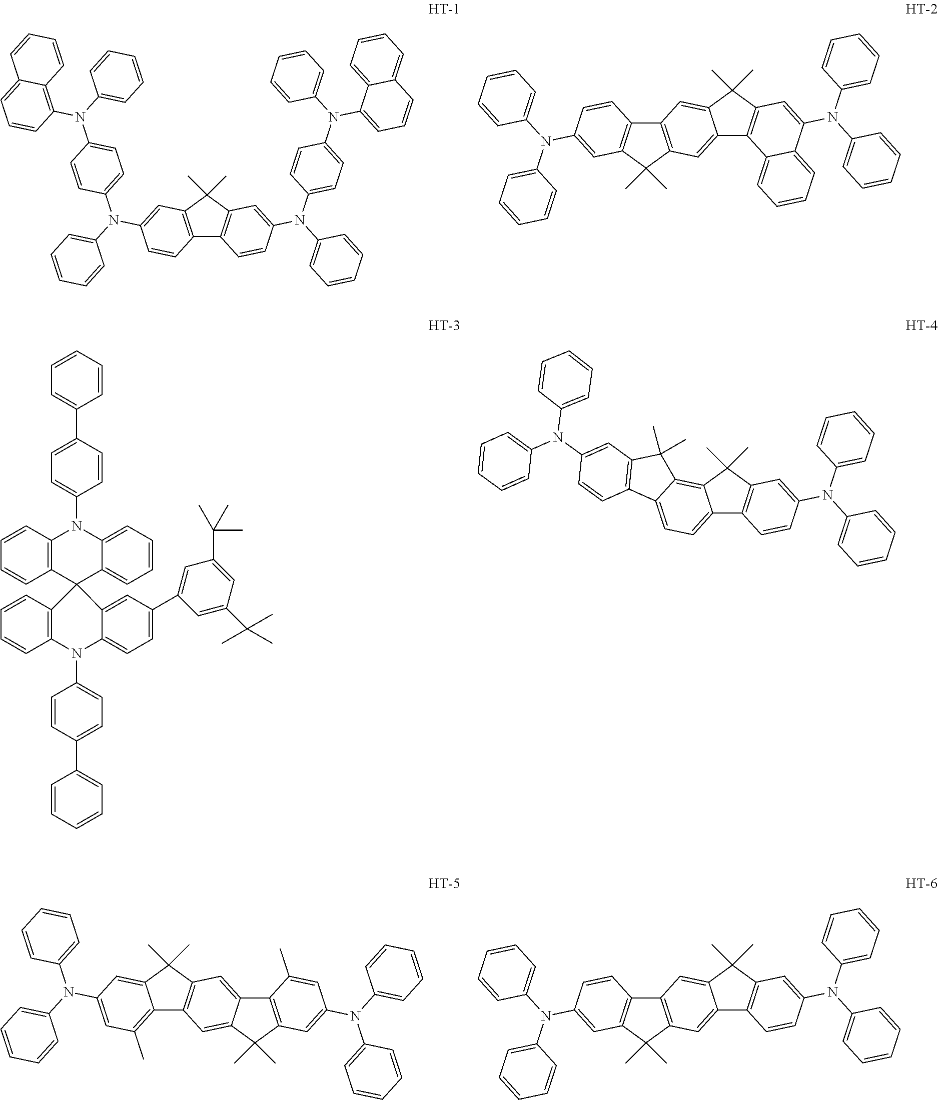

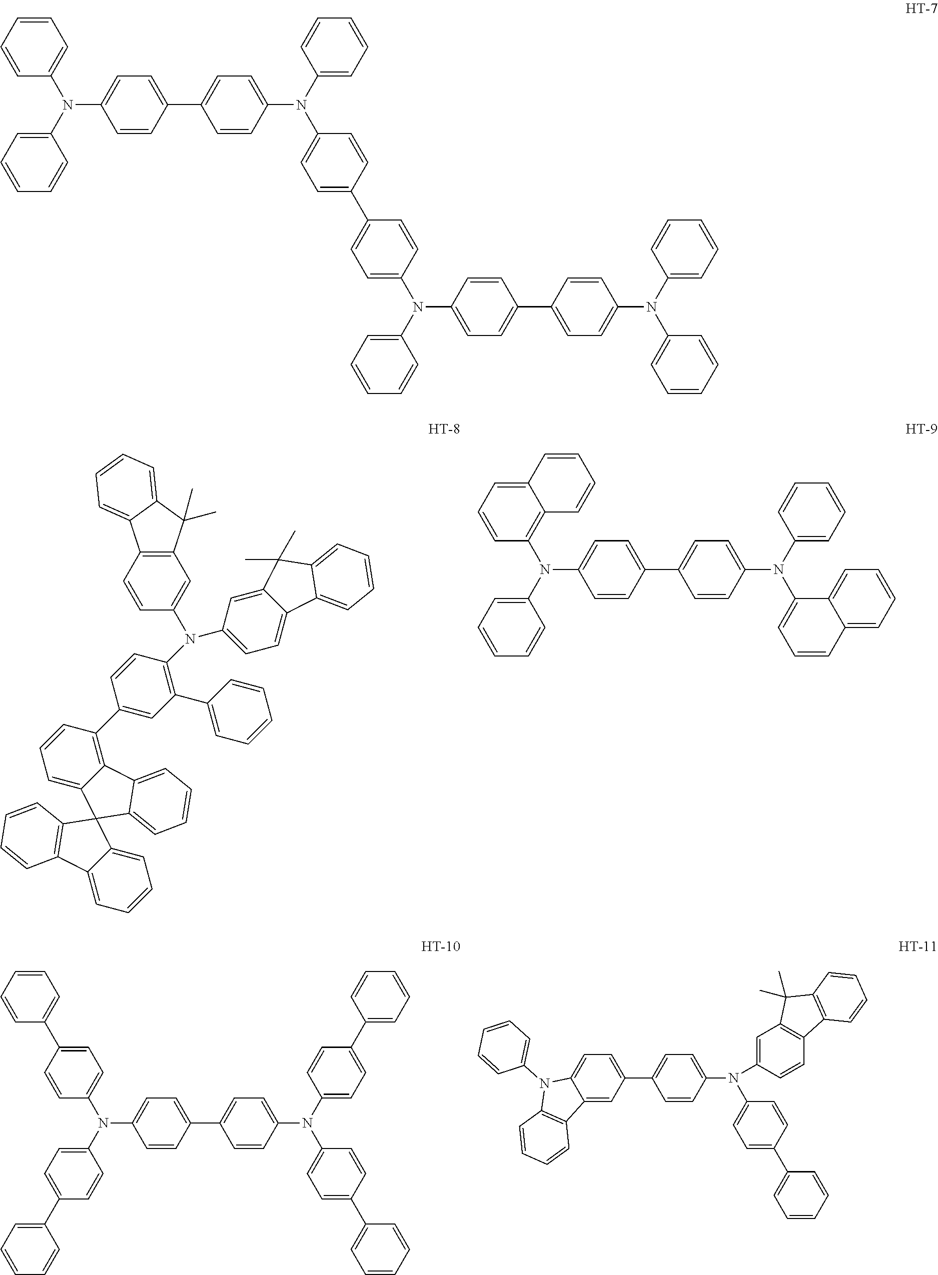

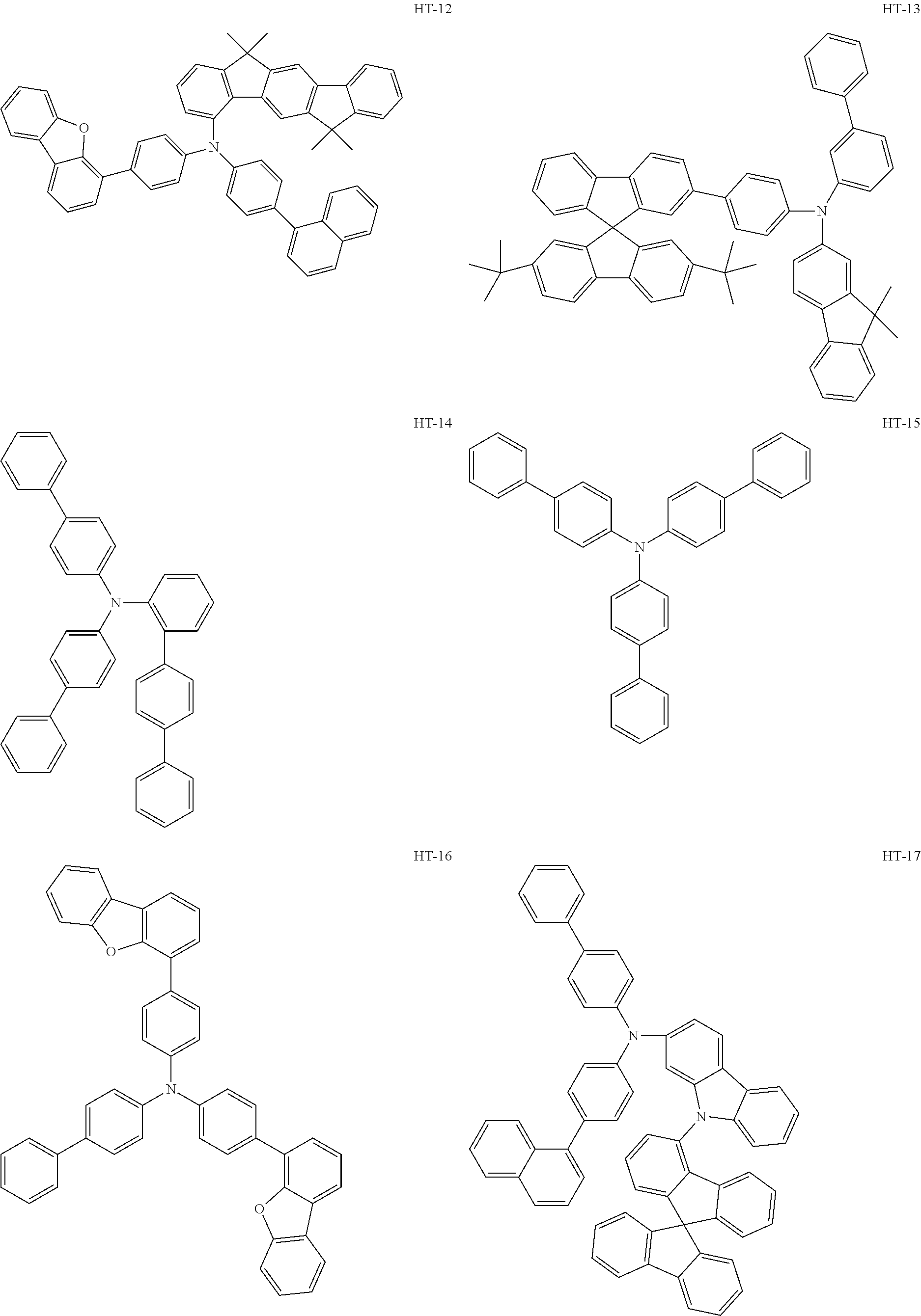

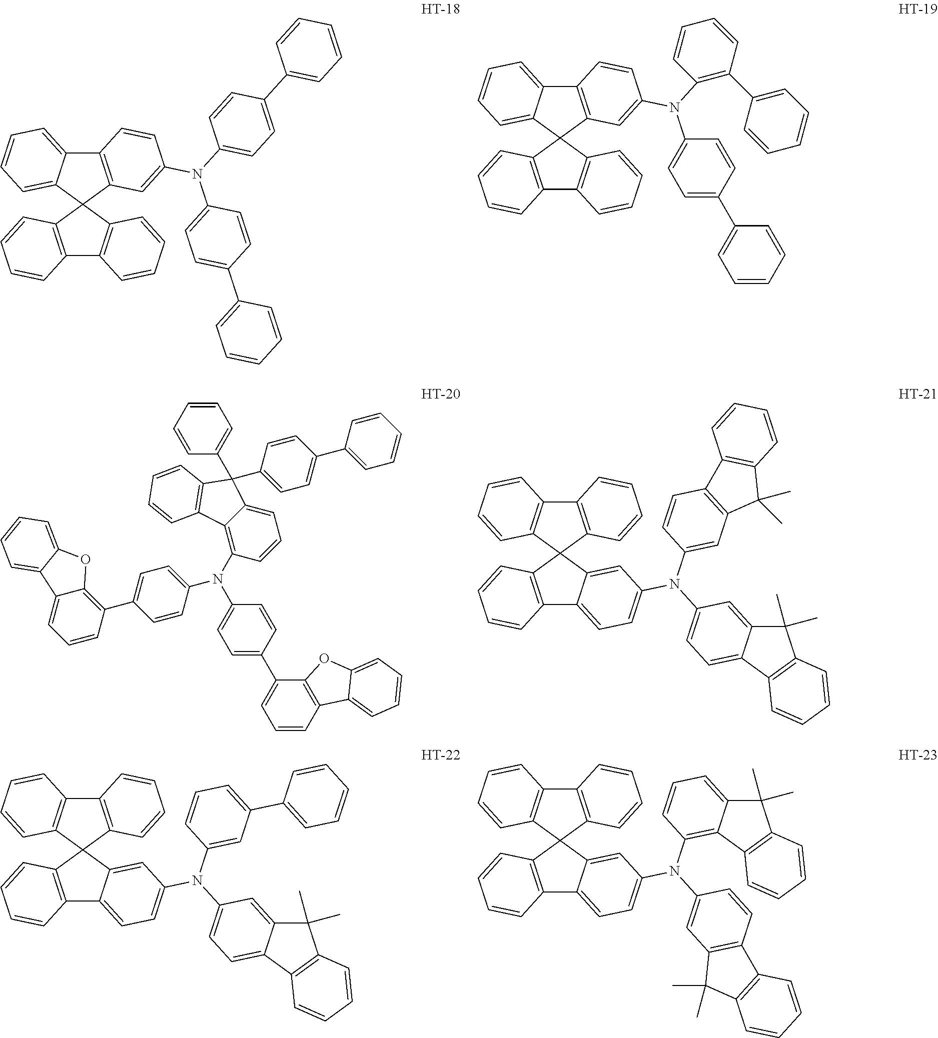

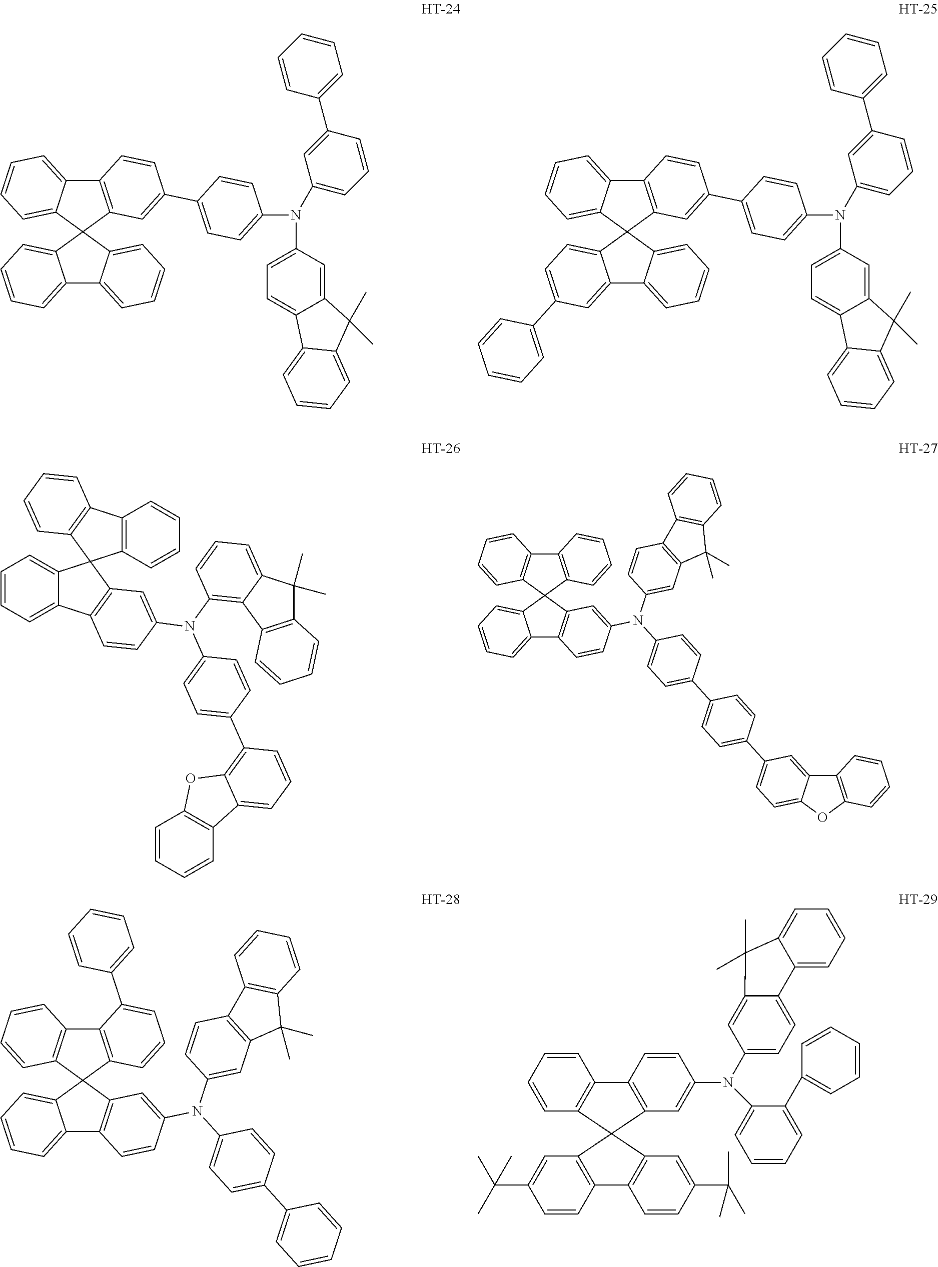

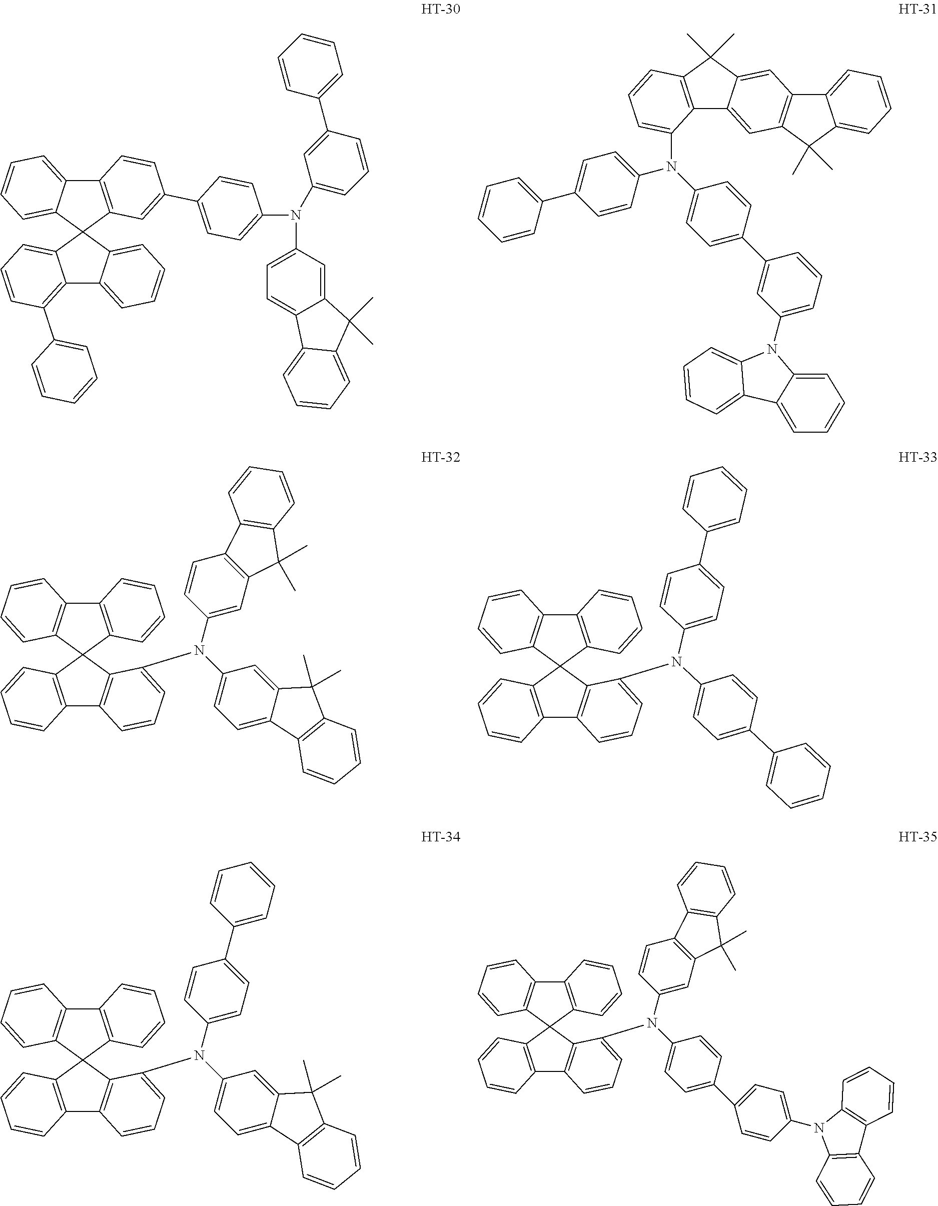

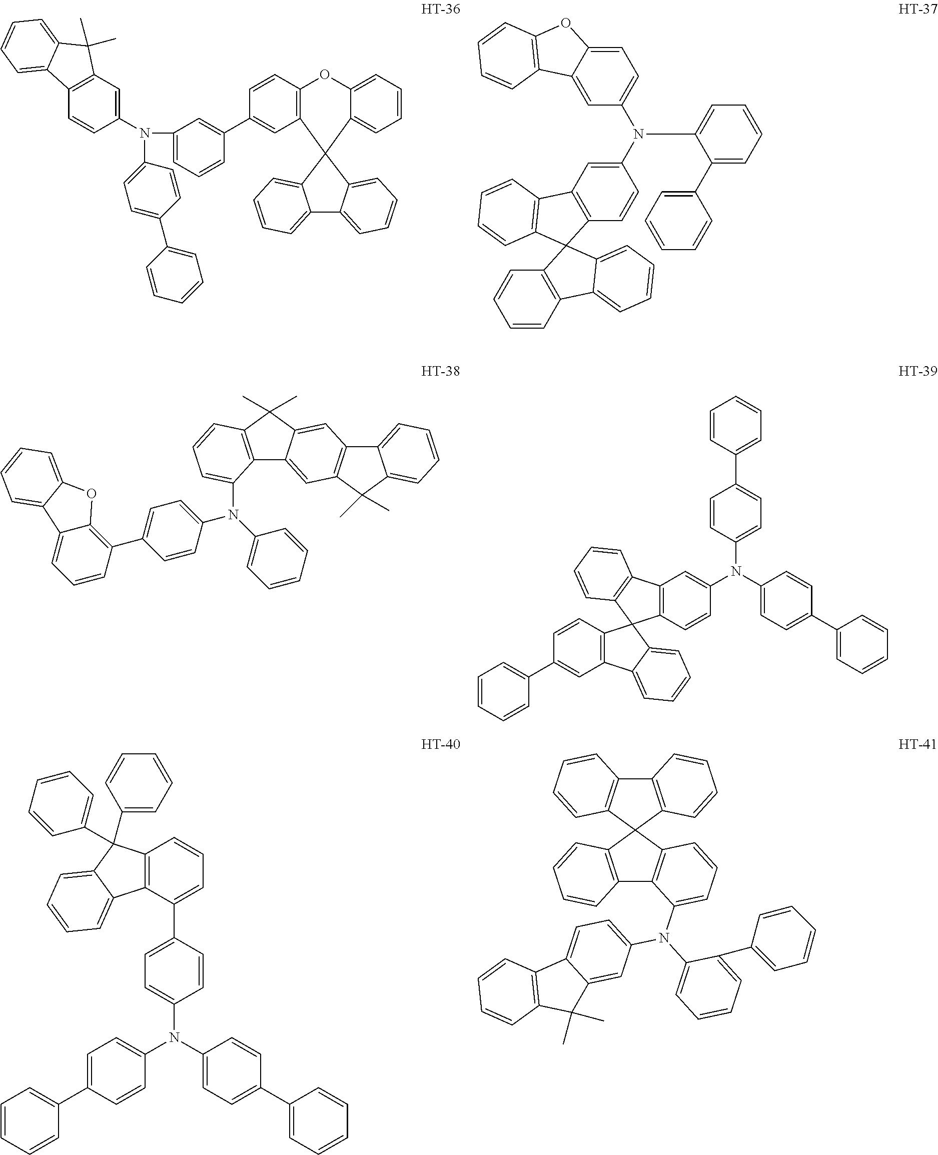

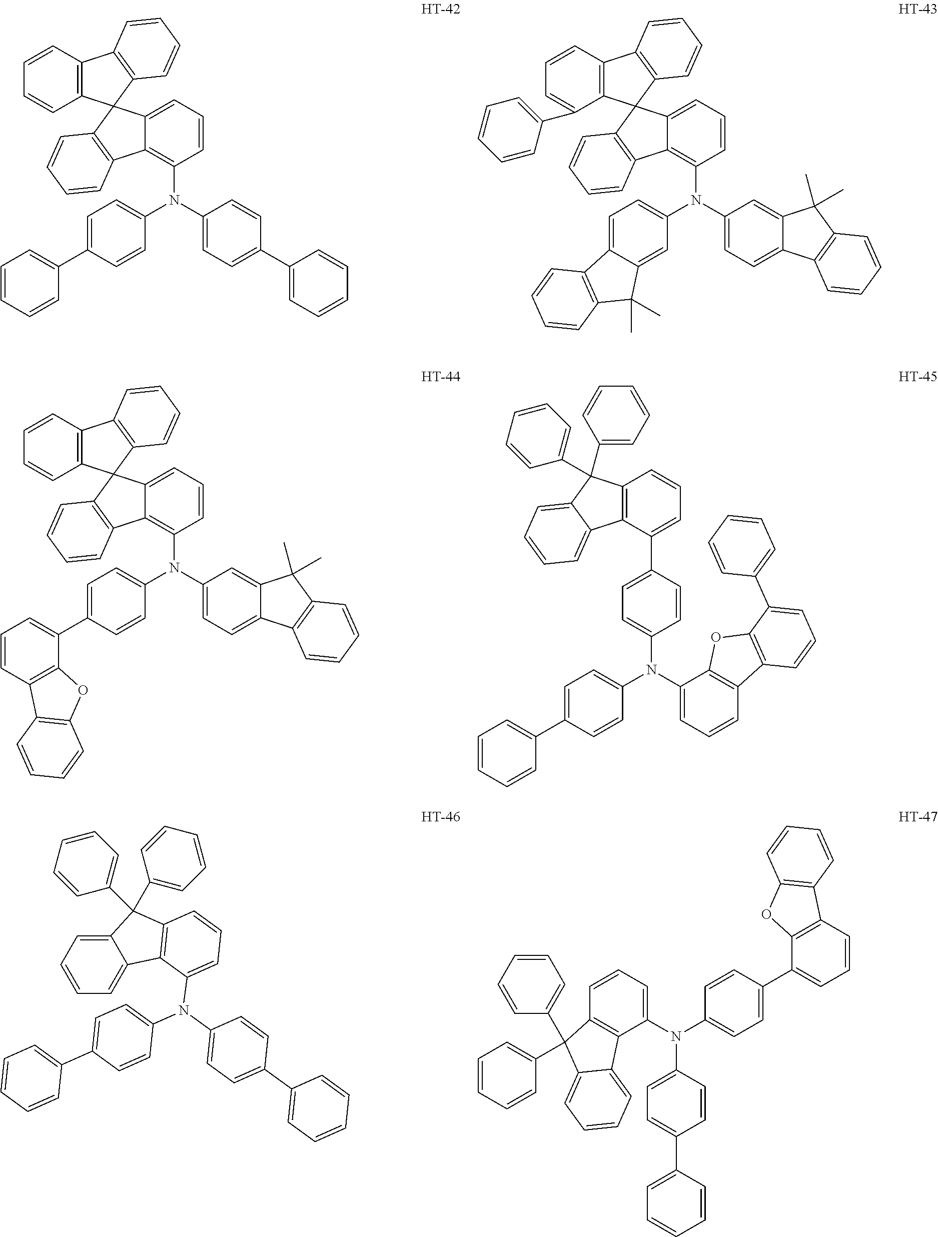

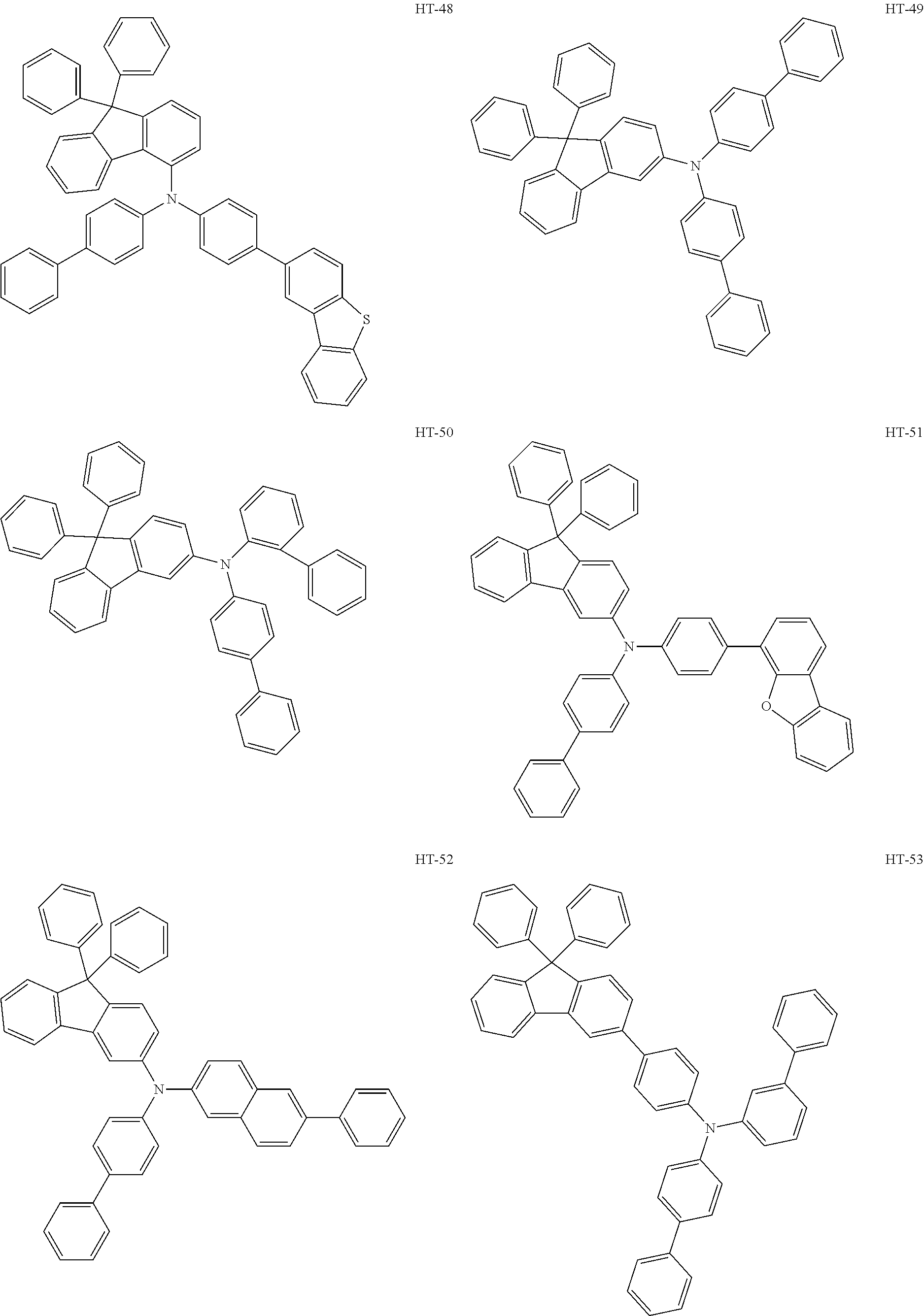

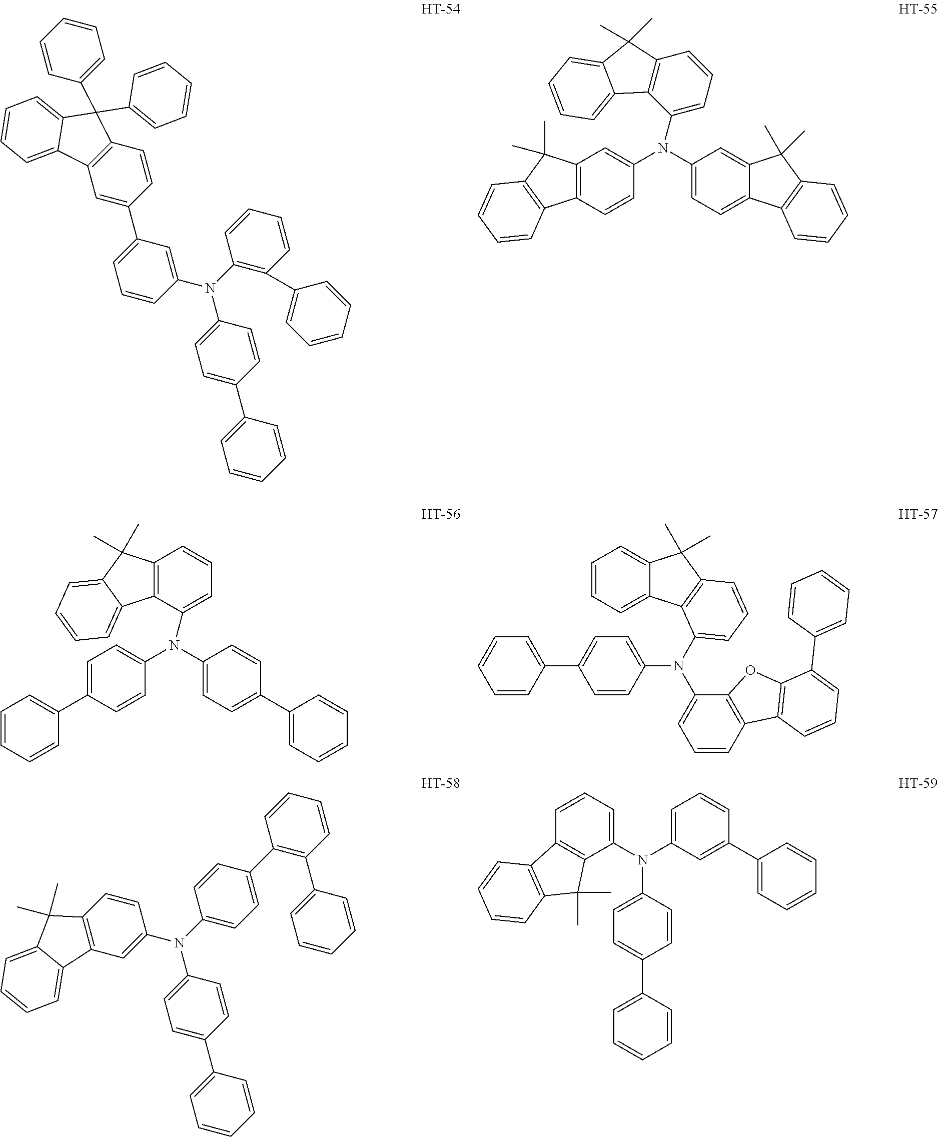

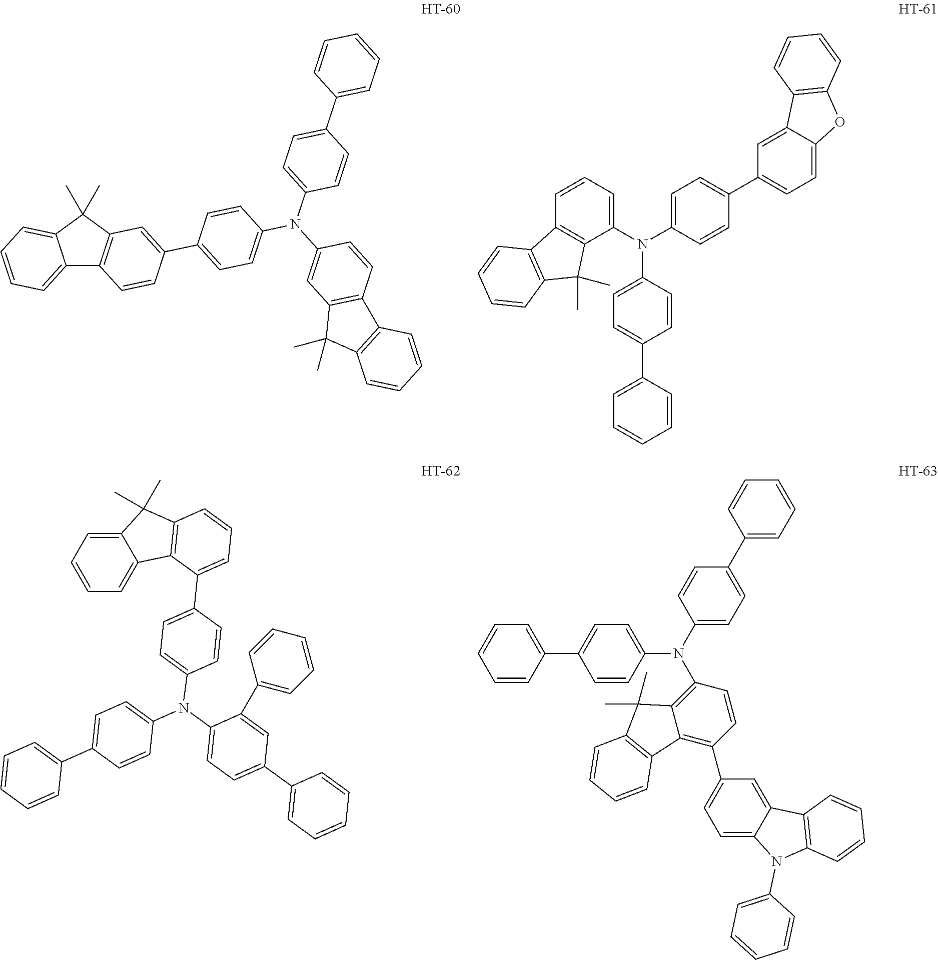 [SMILES: CC1(C)c2ccccc2-c2ccc(-c3ccc(N(c4ccc(-c5ccccc5)cc4)c4ccc5c(c4)C(C)(C)c4ccccc4-5)cc3)cc21 ; CC1(C)c2ccccc2-c2cccc(N(c3ccc(-c4ccccc4)cc3)c3ccc(-c4ccc5oc6ccccc6c5c4)cc3)c21 ; CC1(C)c2ccccc2-c2c(-c3ccc(N(c4ccc(-c5ccccc5)cc4)c4ccc(-c5ccccc5)cc4-c4ccccc4)cc3)cccc21 ; CC1(C)c2ccccc2-c2c(-c3ccc4c(c3)c3ccccc3n4-c3ccccc3)ccc(N(c3ccc(-c4ccccc4)cc3)c3ccc(-c4ccccc4)cc3)c21]

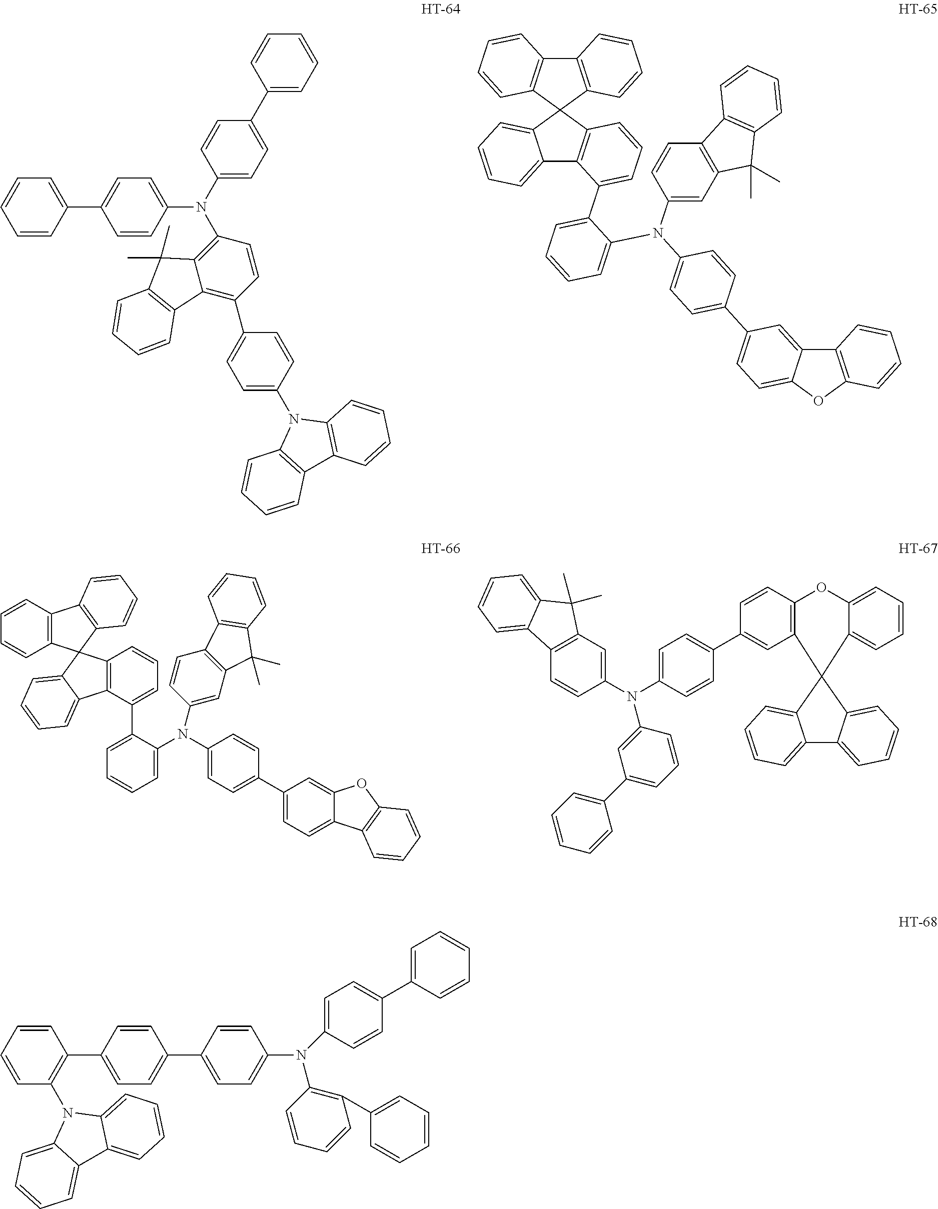

The compounds HT-1 to HT-68 are therefore generally of excellent suitability for the abovementioned uses in OLEDs of any design and composition, not just in OLEDs according to the present application.

Processes for producing these compounds and further relevant disclosure relating to the use of these compounds is disclosed in the following publications: WO 2021/074106, WO 2018/069167, WO 2020/127145, WO 2019/048443, WO 2012/034627, WO 2019/020654, WO 2014/079527, WO 2013/120577, and WO 2015/158411. The compounds show good performance data in OLEDs, especially good lifetime and good efficiency.

Cathode:

Preferred cathodes of the electronic device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Anode:

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (organic solar cell) or the emission of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

In a preferred embodiment, the electronic device is characterized in that one or more layers are coated by a sublimation process. In this case, the materials are applied by vapour deposition in vacuum sublimation systems at an initial pressure of less than 10-5 mbar, preferably less than 10 mbar. In this case, however, it is also possible that the initial pressure is even lower, for example less than 10-7 mbar.

Preference is likewise given to an electronic device, characterized in that one or more layers are coated by the OVPD (organic vapour phase deposition) method or with the aid of a carrier gas sublimation. In this case, the materials are applied at a pressure between 10-5 mbar and 1 bar. A special case of this method is the OVJP (organic vapour jet printing) method, in which the materials are applied directly by a nozzle and thus structured (for example M. S. Arnold et al., Appl. Phys. Lett. 2008, 92, 053301).

Preference is additionally given to an electronic device, characterized in that one or more layers are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble compounds of formula (1) are needed. High solubility can be achieved by suitable substitution of the compounds.

It is further preferable that an electronic device of the invention is produced by applying one or more layers from solution and one or more layers by a sublimation method.

After application of the layers, according to the use, the device is structured, contact-connected and finally sealed, in order to rule out damaging effects of water and air.

According to the invention, the electronic devices comprising one or more compounds of formula (1) can be used in displays, as light sources in lighting applications and as light sources in medical and/or cosmetic applications.

The compounds of the invention and the organic electroluminescent devices of the invention feature the following advantages over the prior art:
1. The compounds of the invention are of very good suitability for use in the electron blocker layer (EBL) of an organic electronic device. Devices comprising the compounds of the invention for this purpose feature very good lifetimes. In addition, the efficiencies and operating voltages of such devices are at a very good level. Particularly good performance data are achieved when the electronic device has green emission, i.e. the device emits green light on account of fluorescent or phosphorescent emission, preferably phosphorescent emission.
2. The compounds of the invention are of very good suitability for use in a hole transport layer or a hole injection layer in electronic devices, for example in organic electroluminescent devices, especially on account of their high hole mobility.
3. The compounds of the invention have a relatively low sublimation temperature, high thermal stability, and high oxidation stability and a high glass transition temperature, which is advantageous for processability, for example from solution or from the gas phase, and also for use in electronic devices. On the basis of their performance data and the other physical properties mentioned, the compounds are eminently suitable for commercial use, for commercial production and for mass production.
4. The use of the compounds of the invention in electronic devices as a hole-conducting matrix or as a hole transport or hole injection material leads to high efficiencies, low operating voltages and long lifetimes.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Thus, any feature disclosed in the present invention, unless stated otherwise, should be considered as an example of a generic series or as an equivalent or similar feature.

All features of the present invention may be combined with one another in any manner, unless particular features and/or steps are mutually exclusive. This is especially true of preferred features of the present invention. Equally, features of non-essential combinations may be used separately (and not in combination).

It should also be pointed out that many of the features, and especially those of the preferred embodiments of the present invention, should themselves be regarded as inventive and not merely as some of the embodiments of the present invention. For these features, independent protection may be sought in addition to or as an alternative to any currently claimed invention.

The technical teaching disclosed with the present invention may be abstracted and combined with other examples.

The invention is illustrated in more detail by the examples which follow, without any intention of restricting it thereby.

### EXAMPLES

### A) Synthesis Examples

Quite generally, the compounds of the invention are synthesized by methods that are very well known to the person skilled in the art in the field. First of all, the fluorene is converted by means of Suzuki coupling. In a second step, the product from the first reaction is converted to the ultimate product by means of a Buchwald reaction.

### a)₃-(4-Chlorophenyl)-9,9-dimethyl-9H-fluorene

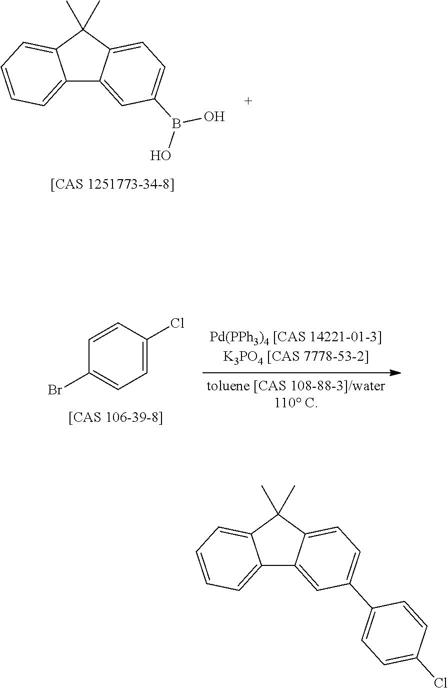 [SMILES: CC1(C)c2ccccc2-c2cc(B(O)O)ccc21 ; Clc1ccc(Br)cc1 ; CC1(C)c2ccccc2-c2cc(-c3ccc(Cl)cc3)ccc21]

39.5 g (166 mmol, 1.10 eq) of (9,9-dimethyl-9H-fluoren-3-yl)boronic acid [CAS 1251773-34-8], 30.6 g (160 mmol, 1.00 eq) of 1-bromo-4-chlorobenzene [CAS 106-39-8] and 102 g (480 mmol; 3.00 eq) of potassium phosphate [CAS 7778-53-2] are dissolved in 2000 ml of toluene [CAS 108-88-3] and 200 ml of water. After inertization in an argon stream for 45 minutes, 3.70 g (3.20 mmol, 2.00 mol %) of tetrakis(triphenylphosphine)palladium is added and the mixture is heated to reflux for 16 hours. After cooling to room temperature, the organic phase is removed and the aqueous phase is extracted with ethyl acetate. The combined organic phases are washed with water and dried over Na₂SO₄. After the solvent has been removed under reduced pressure, the solids obtained are taken up in methylene chloride and precipitated by addition of ethanol. After this operation has been performed repeatedly, 40.5 g (133 mmol, 83% of theory) of the product is obtained.

The following are obtained analogously:

| No. | Reactant 1 | Reactant 2 | Product | Yield |
|---|---|---|---|---|
| 1a | 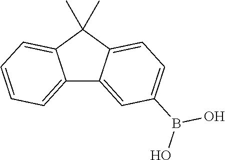 [SMILES: CC1(C)c2ccccc2-c2cc(B(O)O)ccc21] | 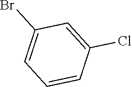 | 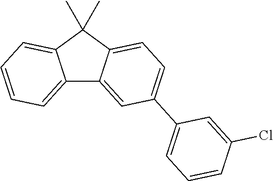 [SMILES: CC1(C)c2ccccc2-c2cc(-c3cccc(Cl)c3)ccc21] | 80% |
| | [CAS 1251773-34-8] | [CAS 108-37-2] | | |
| 2a | 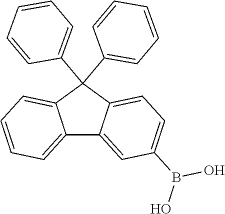 [SMILES: OB(O)c1ccc2c(c1)-c1ccccc1C2(c1ccccc1)c1ccccc1] | 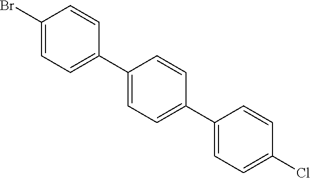 [SMILES: Clc1ccc(-c2ccc(-c3ccc(Br)cc3)cc2)cc1] | 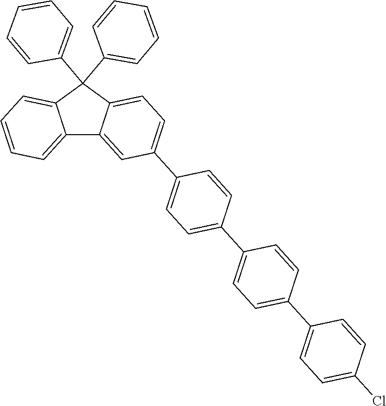 [SMILES: Clc1ccc(-c2ccc(-c3ccc(-c4ccc5c(c4)-c4ccccc4C5(c4ccccc4)c4ccccc4)cc3)cc2)cc1] | 68% |
| | [CAS 1635401-03-4] | [CAS 2413379-08-3] | | |
| 3a | 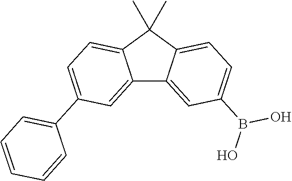 [SMILES: CC1(C)c2ccc(B(O)O)cc2-c2cc(-c3ccccc3)ccc21] | 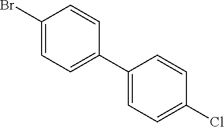 [SMILES: Clc1ccc(-c2ccc(Br)cc2)cc1] | 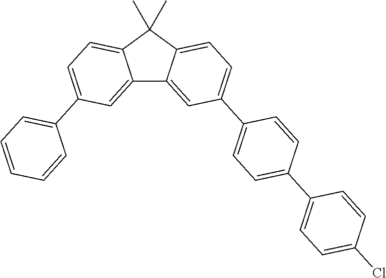 [SMILES: CC1(C)c2ccc(-c3ccccc3)cc2-c2cc(-c3ccc(-c4ccc(Cl)cc4)cc3)ccc21] | 72% |
| | [CAS 1319162-39-4] | [CAS 23055-77-8] | | |
| 4a | 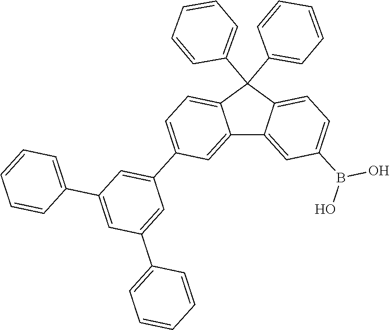 [SMILES: OB(O)c1ccc2c(c1)-c1cc(-c3cc(-c4ccccc4)cc(-c4ccccc4)c3)ccc1C2(c1ccccc1)c1ccccc1] | 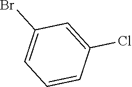 | 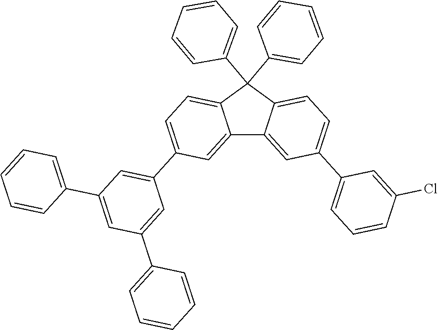 [SMILES: Clc1cccc(-c2ccc3c(c2)-c2cc(-c4cc(-c5ccccc5)cc(-c5ccccc5)c4)ccc2C3(c2ccccc2)c2ccccc2)c1] | 55% |
| | [CAS 1319162-42-9] | [CAS 108-37-2] | | |
| 5a | 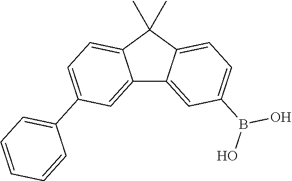 [SMILES: CC1(C)c2ccc(B(O)O)cc2-c2cc(-c3ccccc3)ccc21] | 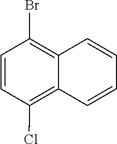 [SMILES: Clc1ccc(Br)c2ccccc12] | 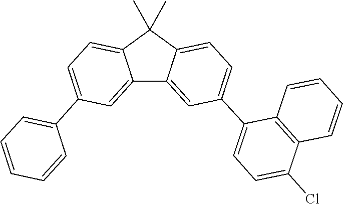 [SMILES: CC1(C)c2ccc(-c3ccccc3)cc2-c2cc(-c3ccc(Cl)c4ccccc34)ccc21] | 61% |
| | [CAS 1319162-39-4] | [CAS 53220-82-9] | | |
| 6a | 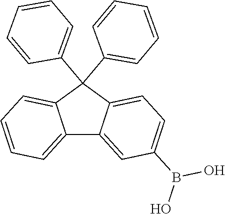 [SMILES: OB(O)c1ccc2c(c1)-c1ccccc1C2(c1ccccc1)c1ccccc1] | 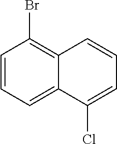 [SMILES: Clc1cccc2c(Br)cccc12] | 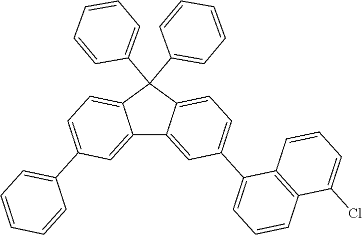 [SMILES: Clc1cccc2c(-c3ccc4c(c3)-c3cc(-c5ccccc5)ccc3C4(c3ccccc3)c3ccccc3)cccc12] | 64% |
| | [CAS 1635401-03-4] | [CAS 77332-65-1] | | |
| 7a | 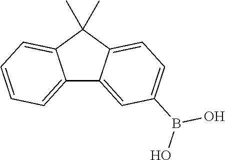 [SMILES: CC1(C)c2ccccc2-c2cc(B(O)O)ccc21] | 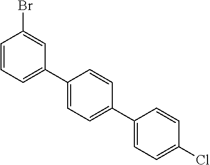 [SMILES: Clc1ccc(-c2ccc(-c3cccc(Br)c3)cc2)cc1] | 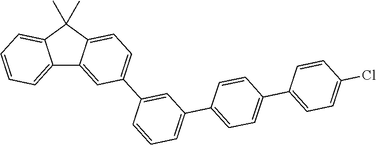 [SMILES: CC1(C)c2ccccc2-c2cc(-c3cccc(-c4ccc(-c5ccc(Cl)cc5)cc4)c3)ccc21] | 70% |
| | [CAS 1251773-34-8] | [CAS 915031-07-1] | | |
| 8a | 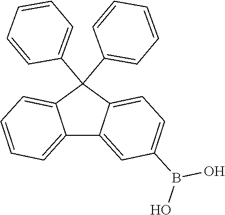 [SMILES: OB(O)c1ccc2c(c1)-c1ccccc1C2(c1ccccc1)c1ccccc1] | 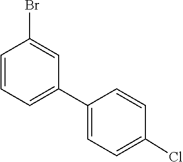 [SMILES: Clc1ccc(-c2cccc(Br)c2)cc1] | 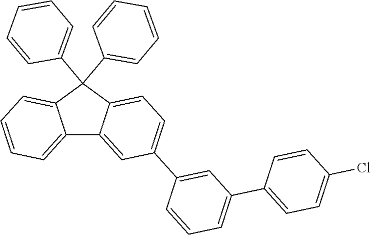 [SMILES: Clc1ccc(-c2cccc(-c3ccc4c(c3)-c3ccccc3C4(c3ccccc3)c3ccccc3)c2)cc1] | 79% |
| | [CAS 1635401-03-4] | [CAS 164334-69-4] | | |
| 9a | 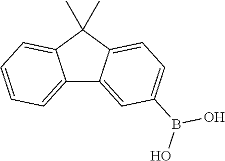 [SMILES: CC1(C)c2ccccc2-c2cc(B(O)O)ccc21] | 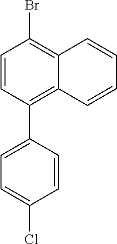 [SMILES: Clc1ccc(-c2ccc(Br)c3ccccc23)cc1] | 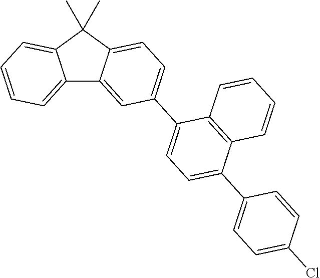 [SMILES: CC1(C)c2ccccc2-c2cc(-c3ccc(-c4ccc(Cl)cc4)c4ccccc34)ccc21] | 69% |
| | [CAS 1251773-34-8] | [CAS 2403429-47-8] | | |
| 10a | 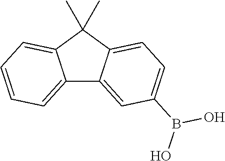 [SMILES: CC1(C)c2ccccc2-c2cc(B(O)O)ccc21] | 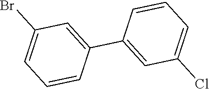 [SMILES: Clc1cccc(-c2cccc(Br)c2)c1] | 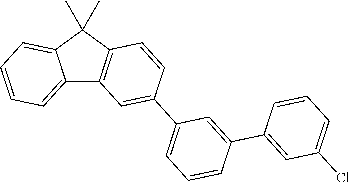 [SMILES: CC1(C)c2ccccc2-c2cc(-c3cccc(-c4cccc(Cl)c4)c3)ccc21] | 71% |
| | [CAS 1251773-34-8] | [CAS 844856-42-4] | | |

### b) N-[4-(9,9-dimethyl-9H-fluoren-3-yl)phenyl]-N-(4-{8-oxatricyclo[7.4.0.02,7]trideca1(9),2(7),3,5,10,12-hexaen-6-yl}phenyl)-[1,1′-biphenyl]-4-amine

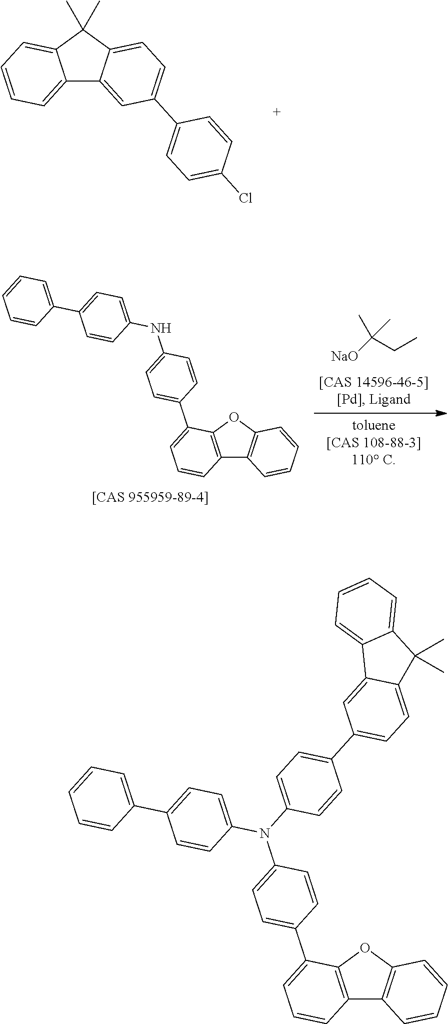

An initial charge of 40.0 g (131 mmol; 1.00 eq.) of 3-(4-chlorophenyl)-9,9-dimethyl-9H-fluorene, 54.0 g (131 mmol; 1.00 eq.) of N-(4-{8-oxatricyclo[7.4.0.0^{2,7}]trideca-1(9),2(7),3,5,10,12-hexaen-6-yl}phenyl)-[1,1′-biphenyl]-4-amine [CAS 955959-89-4] and 15.9 g (144 mmol; 1.10 eq.) of sodium tert-pentoxide [CAS 14593-46-5] in 2000 ml of toluene [CAS 108-88-3] is inertized in an argon stream for 30 minutes. Thereafter, 1.62 mg (3.94 mmol; 3 mol %) of dicyclohexyl-(2′,6′-dimethoxybiphenyl-2-yl)phosphine (SPhos) [CAS 657408-07-6] and 886 mg (3.94 mmol; 3 mol %) of palladium acetate [CAS 3375-31-3] are added and the mixture is heated to reflux for 18 hours. After completion of conversion and cooling to room temperature, water is added to the reaction. After separation of the phases and extraction of the aqueous phase with toluene [CAS 108-88-3], the combined organic phases are concentrated and heptane is added. The precipitated solids are isolated. Purification by means of Soxhlet extraction, recrystallization and vacuum sublimation gives the desired product (52.0 g; 77.1 mmol; 59% of theory).

The following are obtained analogously:

| No | Reactant 1 | Reactant 2 | Product | Yield |
|---|---|---|---|---|
| 1b | 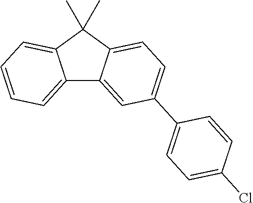 [SMILES: CC1(C)c2ccccc2-c2cc(-c3ccc(Cl)cc3)ccc21] | 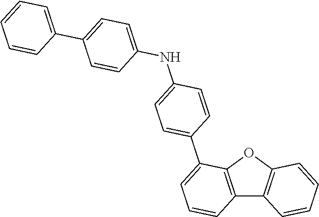 [SMILES: c1ccc(-c2ccc(Nc3ccc(-c4cccc5c4oc4ccccc45)cc3)cc2)cc1] | 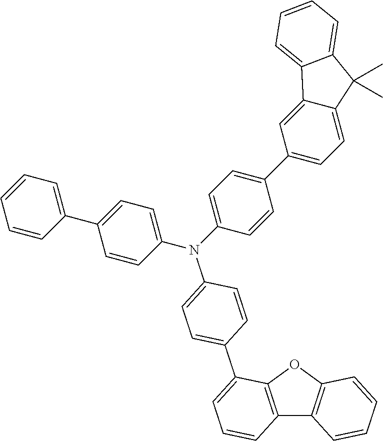 [SMILES: CC1(C)c2ccccc2-c2cc(-c3ccc(N(c4ccc(-c5ccccc5)cc4)c4ccc(-c5cccc6c5oc5ccccc56)cc4)cc3)ccc21] | 56% |
| | | [CAS 955959-89-4] | | |
| 2b | 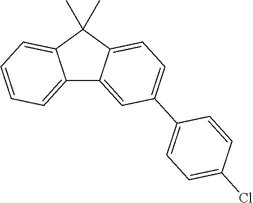 [SMILES: CC1(C)c2ccccc2-c2cc(-c3ccc(Cl)cc3)ccc21] | 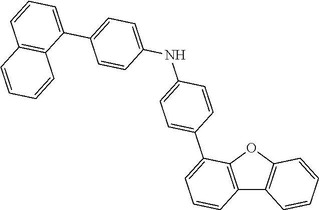 [SMILES: c1ccc2c(-c3ccc(Nc4ccc(-c5cccc6c5oc5ccccc56)cc4)cc3)cccc2c1] | 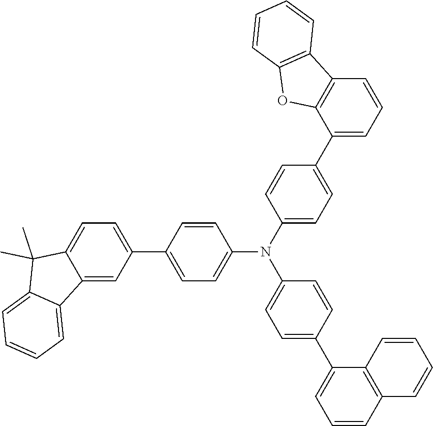 [SMILES: CC1(C)c2ccccc2-c2cc(-c3ccc(N(c4ccc(-c5cccc6ccccc56)cc4)c4ccc(-c5cccc6c5oc5ccccc56)cc4)cc3)ccc21] | 62% |
| | | [CAS 2097371-73-6] | | |
| 3b | 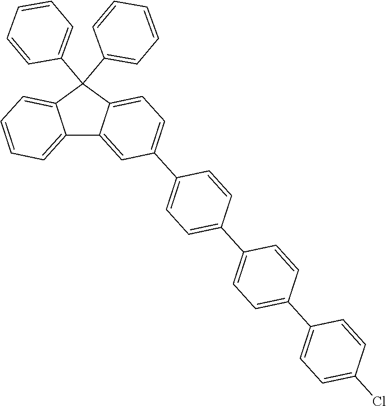 [SMILES: Clc1ccc(-c2ccc(-c3ccc(-c4ccc5c(c4)-c4ccccc4C5(c4ccccc4)c4ccccc4)cc3)cc2)cc1] | 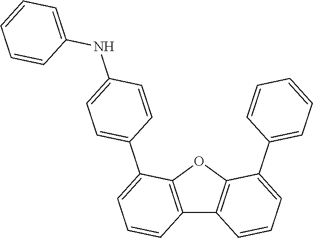 [SMILES: c1ccc(Nc2ccc(-c3cccc4c3oc3c(-c5ccccc5)cccc34)cc2)cc1] | 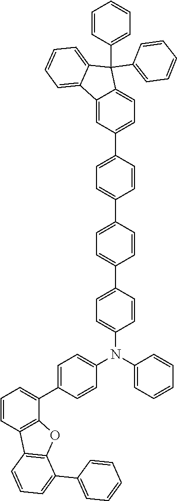 [SMILES: c1ccc(-c2cccc3c2oc2c(-c4ccc(N(c5ccccc5)c5ccc(-c6ccc(-c7ccc(-c8ccc9c(c8)-c8ccccc8C9(c8ccccc8)c8ccccc8)cc7)cc6)cc5)cc4)cccc23)cc1] | 48% |
| | | [CAS 2376742-43-5] | | |
| 4b | 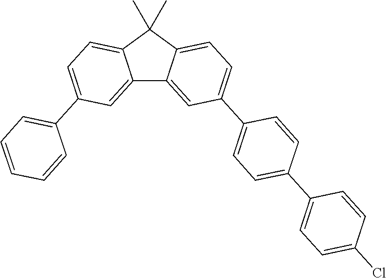 [SMILES: CC1(C)c2ccc(-c3ccccc3)cc2-c2cc(-c3ccc(-c4ccc(Cl)cc4)cc3)ccc21] | 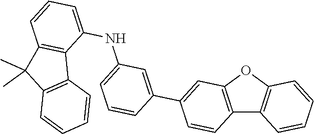 [SMILES: CC1(C)c2ccccc2-c2c(Nc3cccc(-c4ccc5c(c4)oc4ccccc45)c3)cccc21] | 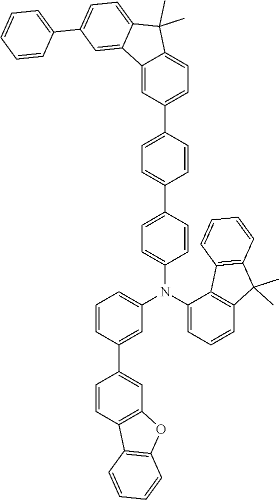 [SMILES: CC1(C)c2ccc(-c3ccccc3)cc2-c2cc(-c3ccc(-c4ccc(N(c5cccc(-c6ccc7c(c6)oc6ccccc67)c5)c5cccc6c5-c5ccccc5C6(C)C)cc4)cc3)ccc21] | 42% |
| | | [CAS 2378356-50-2] | | |
| 5b | 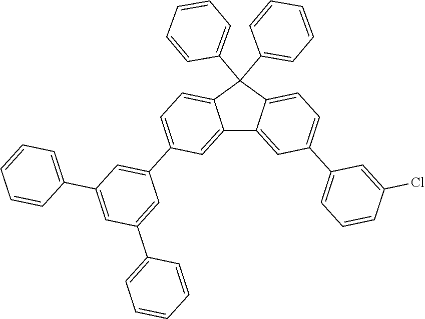 [SMILES: Clc1cccc(-c2ccc3c(c2)-c2cc(-c4cc(-c5ccccc5)cc(-c5ccccc5)c4)ccc2C3(c2ccccc2)c2ccccc2)c1] | 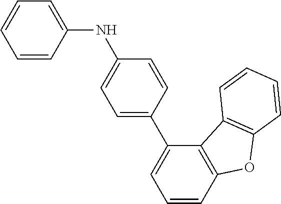 [SMILES: c1ccc(Nc2ccc(-c3cccc4oc5ccccc5c34)cc2)cc1] | 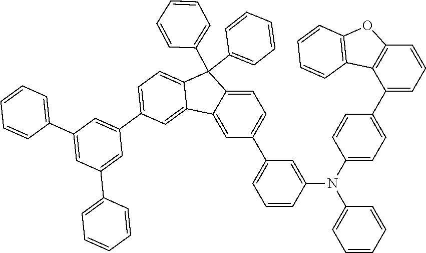 [SMILES: c1ccc(-c2cc(-c3ccccc3)cc(-c3ccc4c(c3)-c3cc(-c5cccc(N(c6ccccc6)c6ccc(-c7cccc8oc9ccccc9c78)cc6)c5)ccc3C4(c3ccccc3)c3ccccc3)c2)cc1] | 55% |
| | | [CAS 2244106-56-5] | | |
| 6b | 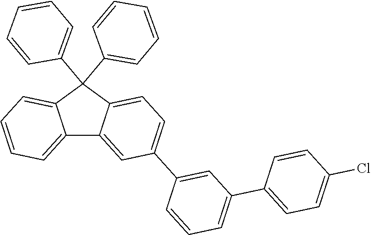 [SMILES: Clc1ccc(-c2cccc(-c3ccc4c(c3)-c3ccccc3C4(c3ccccc3)c3ccccc3)c2)cc1] | 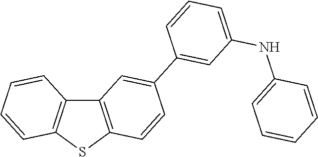 [SMILES: c1ccc(Nc2cccc(-c3ccc4sc5ccccc5c4c3)c2)cc1] | 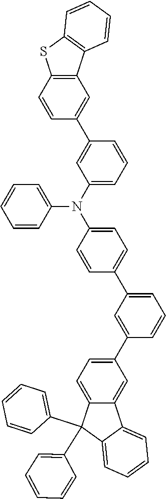 [SMILES: c1ccc(N(c2ccc(-c3cccc(-c4ccc5c(c4)-c4ccccc4C5(c4ccccc4)c4ccccc4)c3)cc2)c2cccc(-c3ccc4sc5ccccc5c4c3)c2)cc1] | 58% |
| | | [CAS 2173402-40-7] | | |
| 7b | 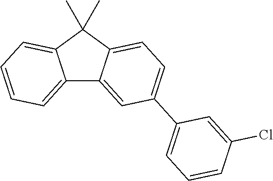 [SMILES: CC1(C)c2ccccc2-c2cc(-c3cccc(Cl)c3)ccc21] | 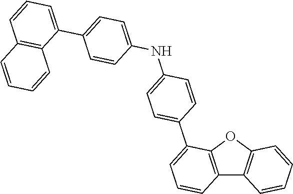 [SMILES: c1ccc2c(-c3ccc(Nc4ccc(-c5cccc6c5oc5ccccc56)cc4)cc3)cccc2c1] | 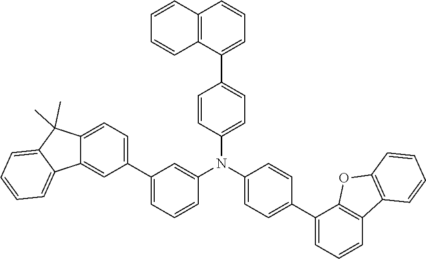 [SMILES: CC1(C)c2ccccc2-c2cc(-c3cccc(N(c4ccc(-c5cccc6ccccc56)cc4)c4ccc(-c5cccc6c5oc5ccccc56)cc4)c3)ccc21] | 63% |
| | | [CAS 2097371-73-6] | | |
| 8b | 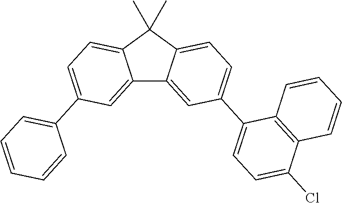 [SMILES: CC1(C)c2ccc(-c3ccccc3)cc2-c2cc(-c3ccc(Cl)c4ccccc34)ccc21] | 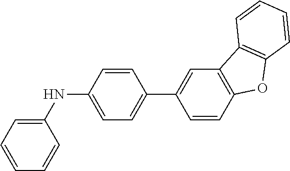 [SMILES: c1ccc(Nc2ccc(-c3ccc4oc5ccccc5c4c3)cc2)cc1] | 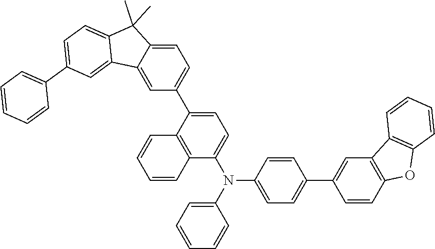 [SMILES: CC1(C)c2ccc(-c3ccccc3)cc2-c2cc(-c3ccc(N(c4ccccc4)c4ccc(-c5ccc6oc7ccccc7c6c5)cc4)c4ccccc34)ccc21] | 51% |
| | | [CAS 1381976-37-9] | | |
| 9b | 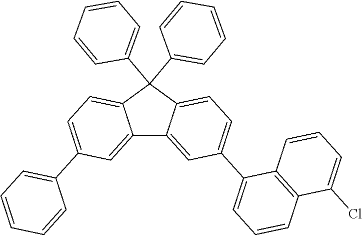 [SMILES: Clc1cccc2c(-c3ccc4c(c3)-c3cc(-c5ccccc5)ccc3C4(c3ccccc3)c3ccccc3)cccc12] | 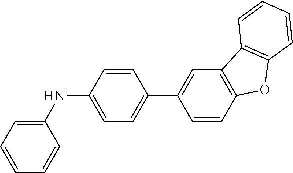 [SMILES: c1ccc(Nc2ccc(-c3ccc4oc5ccccc5c4c3)cc2)cc1] | 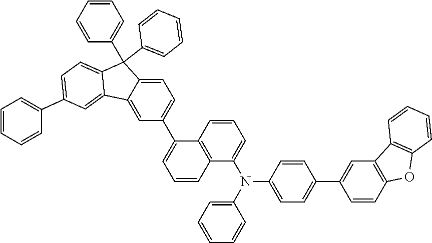 [SMILES: c1ccc(-c2ccc3c(c2)-c2cc(-c4cccc5c(N(c6ccccc6)c6ccc(-c7ccc8oc9ccccc9c8c7)cc6)cccc45)ccc2C3(c2ccccc2)c2ccccc2)cc1] | 57% |
| | | [CAS 1381976-37-9] | | |
| 10 b | 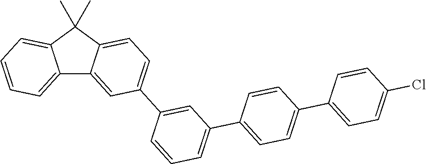 [SMILES: CC1(C)c2ccccc2-c2cc(-c3cccc(-c4ccc(-c5ccc(Cl)cc5)cc4)c3)ccc21] | 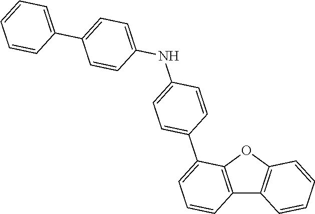 [SMILES: c1ccc(-c2ccc(Nc3ccc(-c4cccc5c4oc4ccccc45)cc3)cc2)cc1] | 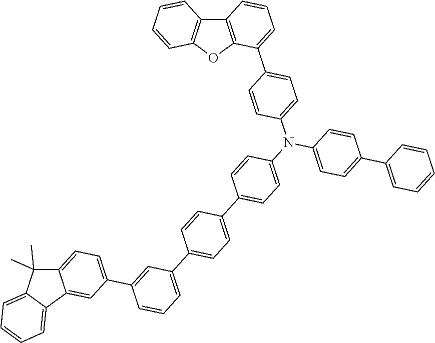 [SMILES: CC1(C)c2ccccc2-c2cc(-c3cccc(-c4ccc(-c5ccc(N(c6ccc(-c7ccccc7)cc6)c6ccc(-c7cccc8c7oc7ccccc78)cc6)cc5)cc4)c3)ccc21] | 45% |
| | | [CAS 955959-89-4] | | |
| 11 b | 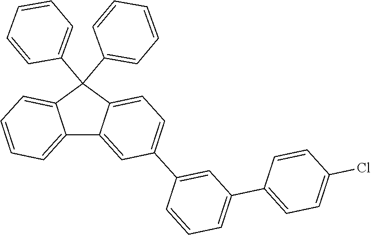 [SMILES: Clc1ccc(-c2cccc(-c3ccc4c(c3)-c3ccccc3C4(c3ccccc3)c3ccccc3)c2)cc1] | 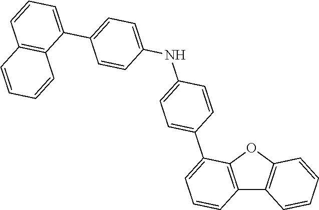 [SMILES: c1ccc2c(-c3ccc(Nc4ccc(-c5cccc6c5oc5ccccc56)cc4)cc3)cccc2c1] | 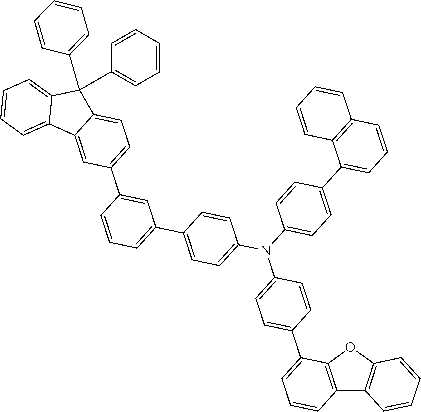 [SMILES: c1ccc(C2(c3ccccc3)c3ccccc3-c3cc(-c4cccc(-c5ccc(N(c6ccc(-c7cccc8ccccc78)cc6)c6ccc(-c7cccc8c7oc7ccccc78)cc6)cc5)c4)ccc32)cc1] | 60% |
| | | [CAS 2097371-73-6] | | |
| 13 b | 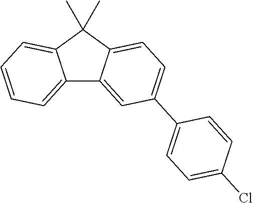 [SMILES: CC1(C)c2ccccc2-c2cc(-c3ccc(Cl)cc3)ccc21] | 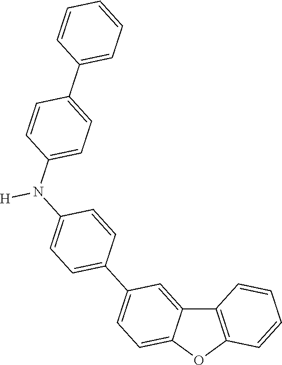 [SMILES: c1ccc(-c2ccc(Nc3ccc(-c4ccc5oc6ccccc6c5c4)cc3)cc2)cc1] | 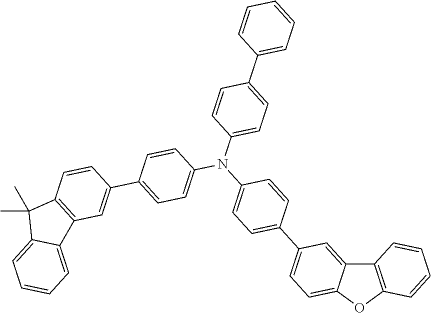 [SMILES: CC1(C)c2ccccc2-c2cc(-c3ccc(N(c4ccc(-c5ccccc5)cc4)c4ccc(-c5ccc6oc7ccccc7c6c5)cc4)cc3)ccc21] | 55% |
| | | [CAS 1547491-60-0] | | |
| 14 b | 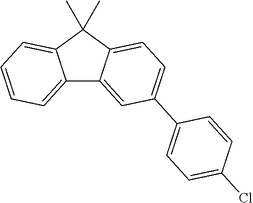 [SMILES: CC1(C)c2ccccc2-c2cc(-c3ccc(Cl)cc3)ccc21] | 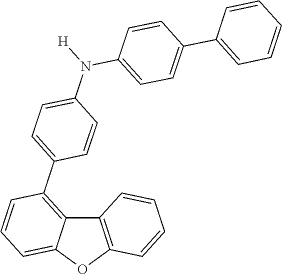 [SMILES: c1ccc(-c2ccc(Nc3ccc(-c4cccc5oc6ccccc6c45)cc3)cc2)cc1] | 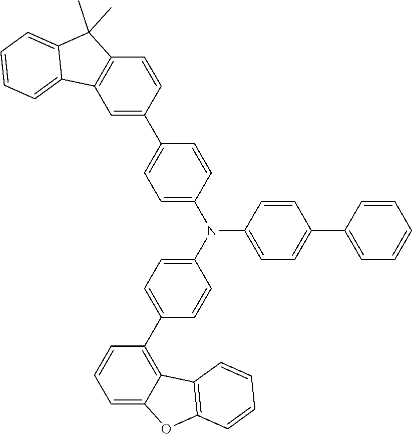 [SMILES: CC1(C)c2ccccc2-c2cc(-c3ccc(N(c4ccc(-c5ccccc5)cc4)c4ccc(-c5cccc6oc7ccccc7c56)cc4)cc3)ccc21] | 52% |
| | | [CAS 1922919-50-3] | | |
| 15 b | 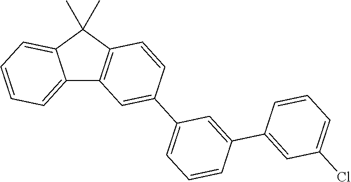 [SMILES: CC1(C)c2ccccc2-c2cc(-c3cccc(-c4cccc(Cl)c4)c3)ccc21] | 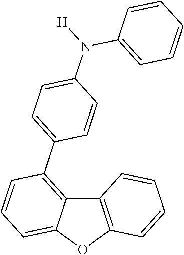 [SMILES: c1ccc(Nc2ccc(-c3cccc4oc5ccccc5c34)cc2)cc1] | 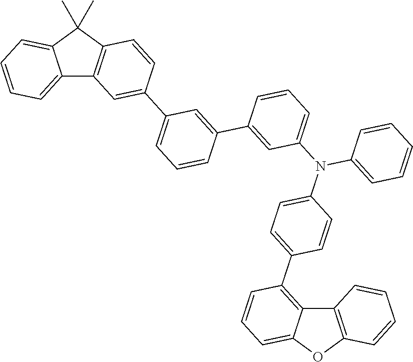 [SMILES: CC1(C)c2ccccc2-c2cc(-c3cccc(-c4cccc(N(c5ccccc5)c5ccc(-c6cccc7oc8ccccc8c67)cc5)c4)c3)ccc21] | 63% |
| | | [CAS 2244106-56-5] | | |
| 16 b | 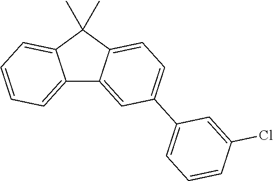 [SMILES: CC1(C)c2ccccc2-c2cc(-c3cccc(Cl)c3)ccc21] | 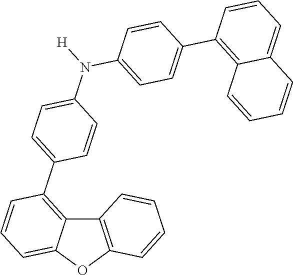 [SMILES: c1ccc2c(-c3ccc(Nc4ccc(-c5cccc6oc7ccccc7c56)cc4)cc3)cccc2c1] | 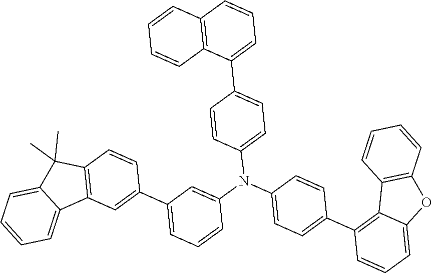 [SMILES: CC1(C)c2ccccc2-c2cc(-c3cccc(N(c4ccc(-c5cccc6ccccc56)cc4)c4ccc(-c5cccc6oc7ccccc7c56)cc4)c3)ccc21] | 60% |
| | | [CAS 2244205-92-1] | | |

The synthesis of the comparative compound, FIMA1, is disclosed in WO2014/015935 A2 (compound 2-8 in example 2).

### B) Device Examples

Examples E1 to E5 which follow (see table 1) present the use of the materials of the invention in OLEDs.

Pretreatment for Examples E1-E5 Glass plates coated with structured ITO (indium tin oxide) of thickness 50 nm are treated prior to coating with an oxygen plasma, followed by an argon plasma. These plasma-treated glass plates form the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminium layer of thickness 100 nm. The exact structure of the OLEDs can be found in table 1. The materials required for production of the OLEDs are shown in table 2. The data of the OLEDs are listed in table 3.

All materials are applied by thermal vapour deposition in a vacuum chamber. In this case, the emission layer always consists of at least one matrix material (host material) and an emitting dopant (emitter) which is added to the matrix material(s) in a particular proportion by volume by co-evaporation. Details given in such a form as IC1:IC2:TEG1 (59%:29%:12%) mean here that the material IC1 is present in the layer in a proportion by volume of 49%, IC2 in a proportion of 44% and TEG1 in a proportion of 7%.

Analogously, the electron transport layer may also consist of a mixture of two materials.

The OLEDs are characterized in a standard manner. For this purpose, the electroluminescence spectra, the current efficiency (CE, measured in cd/A) and the external quantum efficiency (EQE, measured in %) are determined as a function of luminance, calculated from current-voltage-luminance characteristics assuming Lambertian emission characteristics, as is the lifetime. The electroluminescence spectra are determined at a luminance of 1000 cd/m², and the CIE 1931 x and y colour coordinates are calculated therefrom. The parameter U1000 in table 3 refers to the voltage which is required for a luminance of 1000 cd/m. CE1000 and EQE1000 respectively denote the current efficiency and external quantum efficiency that are attained at 1000 cd/m².

The lifetime LT is defined as the time after which the luminance drops from the starting luminance to a certain proportion L1 in the course of operation with constant current density j0. A figure of L1=80% in table 3 means that the lifetime reported in the LT column corresponds to the time after which the luminance falls to 80% of its starting value.

The inventive compounds EG1, EG2, EG3, EG4 are used in examples E2, E3, E4 and E5 as electron blocker material in phosphorescent green OLEDs. The results are compared with the comparative example E1. Table 3 summarizes the performance data of the OLEDs.

TABLE 1
Structure of the OLEDs

| | HIL | HTL | EBL | EML | HBL | ETL | EIL |
|---|---|---|---|---|---|---|---|
| Ex. | thickness | thickness | thickness | thickness | thickness | thickness | thickness |
| E1 | HATCN | SpMA1 | FIMA1 | IC1:IC2:TEG1 | ST2 | ST2:LiQ (50%:50%) | LIQ |
| | 5 nm | 230 nm | 10 nm | (59%:29%:12%) 30 nm | 10 nm | 30 nm | 1 nm |
| E2 | HATCN | SpMA1 | EG1 | IC1:IC2:TEG1 | ST2 | ST2:LiQ (50%:50%) | LIQ |
| | 5 nm | 230 nm | 10 nm | (59%:29%:12%) 30 nm | 10 nm | 30 nm | 1 nm |
| E3 | HATCN | SpMA1 | EG2 | IC1:IC2:TEG1 | ST2 | ST2:LiQ (50%:50%) | LIQ |
| | 5 nm | 230 nm | 10 nm | (59%:29%:12%) 30 nm | 10 nm | 30 nm | 1 nm |
| E4 | HATCN | SpMA1 | EG3 | IC1:IC2:TEG1 | ST2 | ST2:LiQ (50%:50%) | LIQ |
| | 5 nm | 230 nm | 10 nm | (59%:29%:12%) 30 nm | 10 nm | 30 nm | 1 nm |
| E5 | HATCN | SpMA1 | EG4 | IC1:IC2:TEG1 | ST2 | ST2:LiQ (50%:50%) | LIQ |
| | 5 nm | 230 nm | 10 nm | (59%:29%:12%) 30 nm | 10 nm | 30 nm | 1 nm |

TABLE 2
Structural formulae of the materials for the OLEDs

| | |
|---|---|
| 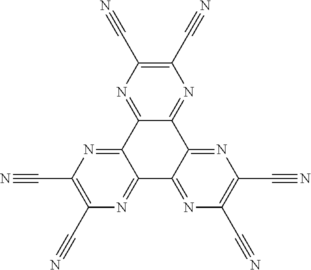 [SMILES: N#Cc1nc2c3nc(C#N)c(C#N)nc3c3nc(C#N)c(C#N)nc3c2nc1C#N] | 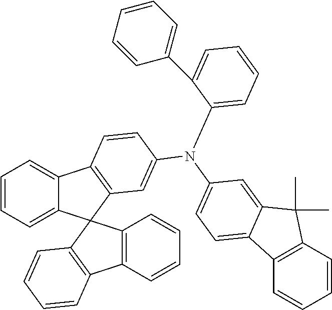 [SMILES: CC1(C)c2ccccc2-c2ccc(N(c3ccc4c(c3)C3(c5ccccc5-c5ccccc53)c3ccccc3-4)c3ccccc3-c3ccccc3)cc21] |
| HATCN | SpMA1 |
| 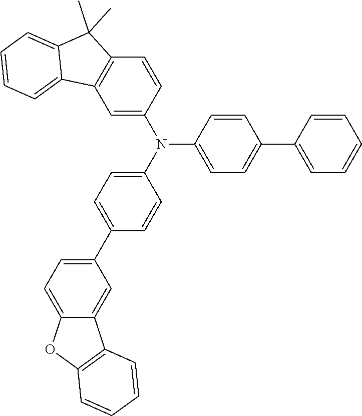 [SMILES: CC1(C)c2ccccc2-c2cc(N(c3ccc(-c4ccccc4)cc3)c3ccc(-c4ccc5oc6ccccc6c5c4)cc3)ccc21] | 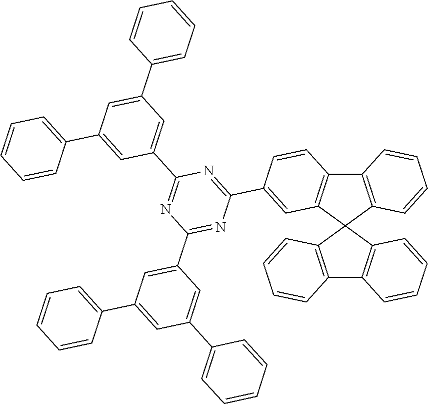 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1] |
| FIMA1 | ST2 |
| 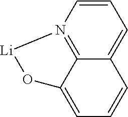 | 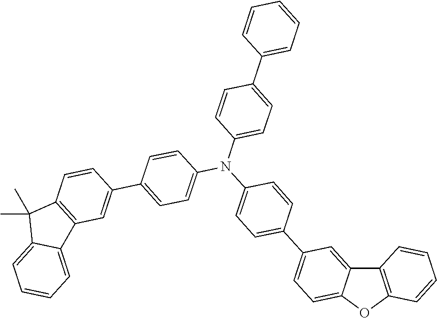 [SMILES: CC1(C)c2ccccc2-c2cc(-c3ccc(N(c4ccc(-c5ccccc5)cc4)c4ccc(-c5ccc6oc7ccccc7c6c5)cc4)cc3)ccc21] |
| LiQ | EG1 |
| 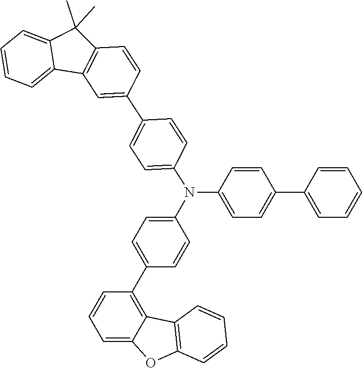 [SMILES: CC1(C)c2ccccc2-c2cc(-c3ccc(N(c4ccc(-c5ccccc5)cc4)c4ccc(-c5cccc6oc7ccccc7c56)cc4)cc3)ccc21] | 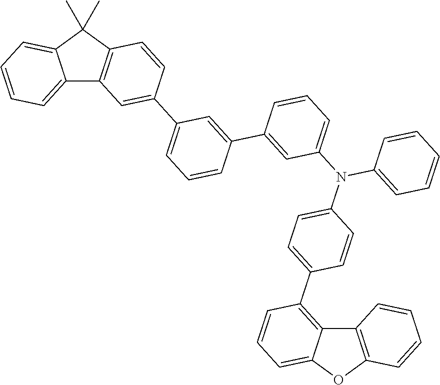 [SMILES: CC1(C)c2ccccc2-c2cc(-c3cccc(-c4cccc(N(c5ccccc5)c5ccc(-c6cccc7oc8ccccc8c67)cc5)c4)c3)ccc21] |
| EG2 | EG3 |
| 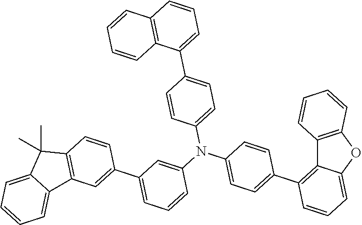 [SMILES: CC1(C)c2ccccc2-c2cc(-c3cccc(N(c4ccc(-c5cccc6ccccc56)cc4)c4ccc(-c5cccc6oc7ccccc7c56)cc4)c3)ccc21] | 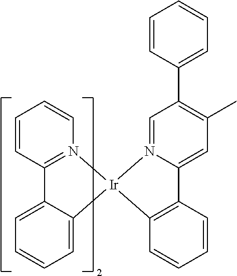 |
| EG4 | TEG1 |
| 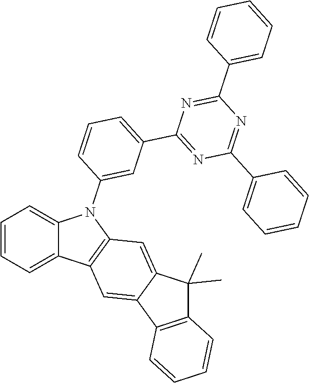 [SMILES: CC1(C)c2ccccc2-c2cc3c4ccccc4n(-c4cccc(-c5nc(-c6ccccc6)nc(-c6ccccc6)n5)c4)c3cc21] | 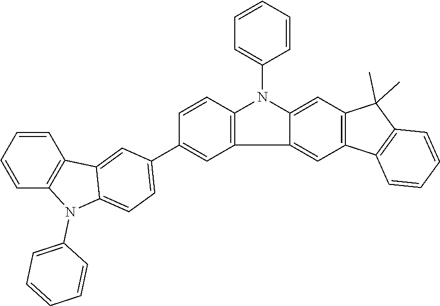 [SMILES: CC1(C)c2ccccc2-c2cc3c4cc(-c5ccc6c(c5)c5ccccc5n6-c5ccccc5)ccc4n(-c4ccccc4)c3cc21] |
| IC1 | IC2 |

TABLE 3
Data of the OLEDs

| | U1000 | SE1000 | EQE 1000 | CIE x/y at | j₀ | L1 | LT |
|---|---|---|---|---|---|---|---|
| Ex. | (V) | (cd/A) | (%) | 1000 cd/m² | (mA/cm²) | (%) | (h) |
| E1 | 3.3 | 69 | 19.8 | 0.36/0.61 | 20 | 80 | 310 |
| E2 | 3.3 | 71 | 20.8 | 0.36/0.62 | 20 | 80 | 328 |
| E3 | 3.3 | 77 | 21.0 | 0.36/0.62 | 20 | 80 | 316 |
| E4 | 3.3 | 80 | 21.5 | 0.36/0.62 | 20 | 80 | 298 |
| E5 | 3.3 | 81 | 21.5 | 0.36/0.62 | 20 | 80 | 305 |

It is found that OLEDs containing the compounds of the invention have very good performance data; in particular, they show distinctly improved efficiencies over the prior art. Moreover, the voltages and lifetimes are at a very high level.

1.-19. (canceled)

20. Compound of formula (1) 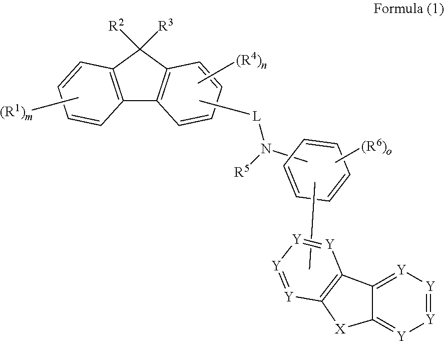
where the symbols used are as follows:
X is O or S;
Y is the same or different at each instance and is CR⁷ or N;
L is a divalent aromatic ring system having 6 to 40 aromatic ring atoms;
R¹, R⁴, R⁶ and R⁷ are the same or different at each instance and are selected from H, D, F, Cl, Br, I, C(═O)R¹¹, CN, Si(R¹¹)₃, N(R¹¹)₂, P(═O)(R¹¹)₂, OR¹¹, S(═O)R¹¹, S(═O)₂R¹¹, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ radicals may be joined to one another and may form a ring and/or two or more R⁴ radicals may be joined to one another and may form a ring and/or two or more R⁶ radicals may be joined to one another and may form a ring and/or two or more R⁷ radicals may be joined to one another and may form a ring; where the alkyl, alkoxy, alkenyl and alkynyl groups mentioned and the aromatic ring systems and heteroaromatic ring systems mentioned may each be substituted by R¹¹ radicals; and where one or more CH₂ groups in the alkyl, alkoxy, alkenyl and alkynyl groups may be replaced by —R¹¹C═CR¹¹—, —C≡C—, Si(R¹¹)₂, C═O, C═NR¹¹, —C(═O)O—, —C(═O)NR¹¹—, NR¹¹, P(═O)(R¹¹), —O—, —S—, SO or SO₂;
R² and R³ are the same or different at each instance and are selected from straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the two R² and R³ radicals may be joined to one another and may form a ring; where the alkyl, alkoxy, alkenyl and alkynyl groups mentioned and the aromatic ring systems and heteroaromatic ring systems mentioned may each be substituted by R¹¹ radicals; and where one or more CH₂ groups in the alkyl, alkoxy, alkenyl and alkynyl groups may be replaced by —R¹¹C═CR¹¹—, —C≡C—, Si(R¹¹)₂, C═O, C═NR¹¹, —C(═O)O—, —C(═O)NR¹¹—, NR¹¹, P(═O)(R¹¹), —O—, —S—, SO or SO₂; if the two R² and R³ radicals form a ring, the result is a spiro compound;
R⁵ is an aromatic ring system which has 6 to 40 aromatic ring atoms and may be substituted by one or more R¹¹ radicals, or a heteroaromatic ring system which has 5 to 40 aromatic ring atoms and may be substituted by one or more R¹¹ radicals;
R¹¹ is the same or different at each instance and is selected from H, D, F, Cl, Br, I, C(═O)R¹², CN, Si(R¹²)₃, N(R¹²)₂, P(═O)(R²)₂, OR¹², S(═O)R¹²,
S(═O)₂R¹², straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹¹ radicals may be joined to one another and may form a ring; where the alkyl, alkoxy, alkenyl and alkynyl groups mentioned and the aromatic ring systems and heteroaromatic ring systems mentioned are each substituted by R¹² radicals; and where one or more CH₂ groups in the alkyl, alkoxy, alkenyl and alkynyl groups mentioned may be replaced by —R¹²C═CR¹²—, —C≡C—, Si(R¹²)₂, C═O, C═NR¹², —C(═O)O—, —C(═O)NR¹²—, NR¹², P(═O)(R²), —O—, —S—, SO or SO₂; where two or more R¹¹ radicals may not form a ring with one another;
R¹² is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where the alkyl, alkoxy, alkenyl and alkynyl groups, aromatic ring systems and heteroaromatic ring systems mentioned may be substituted by one or more radicals selected from F and CN;
m is 0, 1, 2, 3 or 4;
n is 0, 1, 2 or 3;
o is 0, 1, 2 or 3.

21. Compound according to claim 20, characterized in that L is selected from the following formulae: 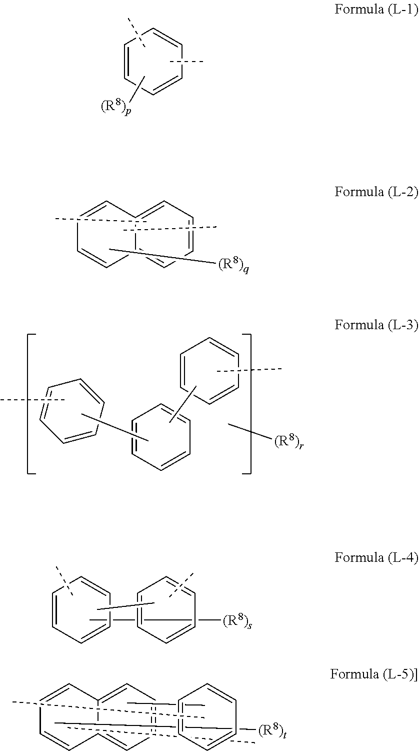
where:
R⁸ is the same or different at each instance and is selected from H, D, F, Cl, Br, I, C(═O)R¹¹, CN, Si(R¹¹)₃, N(R¹¹)₂, P(═O)(R¹¹)₂, OR¹¹, S(═O)R¹¹, S(═O)₂R¹¹, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more Re radicals may be joined to one another and may form a ring; where the alkyl, alkoxy, alkenyl and alkynyl groups mentioned and the aromatic ring systems and heteroaromatic ring systems mentioned may each be substituted by R¹¹ radicals; and where one or more CH₂ groups in the alkyl, alkoxy, alkenyl and alkynyl groups mentioned may be replaced by —R¹¹C═CR¹¹—, —C≡C—, Si(R¹¹)₂, C═O, C═NR¹¹, —C(═O)O—, —C(═O)NR¹¹—, NR¹¹, P(═O)(R¹¹), —O—, —S—, SO or SO₂;
p is 0, 1, 2, 3 or 4;
q is 0, 1, 2, 3, 4, 5 or 6;
r is 0, 1, 2, 3, 4, 5, 7, 8, 9, 10, 11 or 12;
s is 0, 1, 2, 3, 4, 5, 7 or 8;
t is 0, 1, 2, 3, 4, 5, 7, 8, 9 or 10.

22. Compound according to claim 20, characterized in that L is selected from the following formulae:

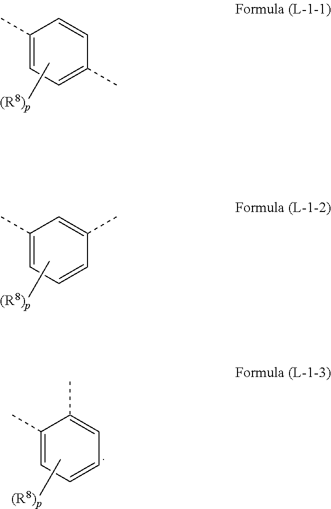

23. Compound according to claim 20, characterized in that L is selected from the following formulae:

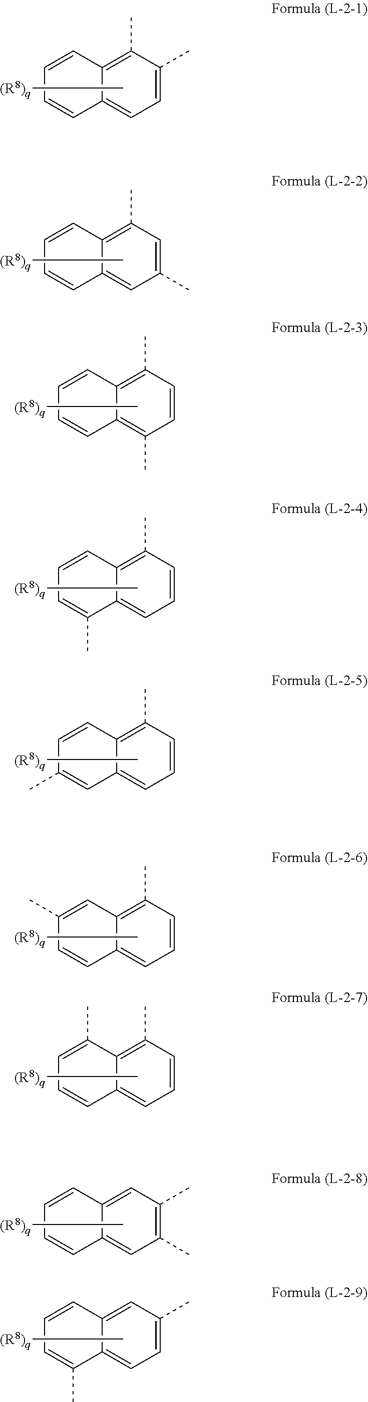

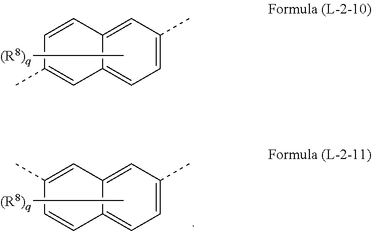

24. Compound according to claim 20, characterized in that L is selected from the following formulae:

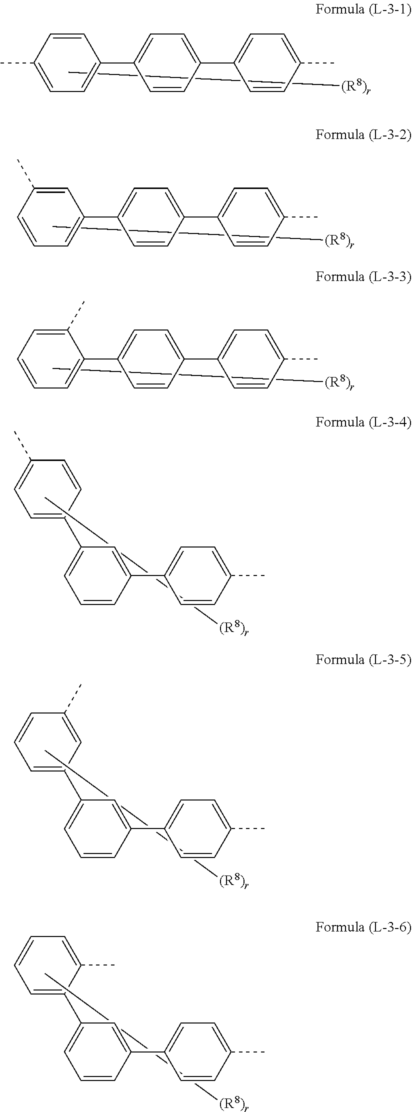

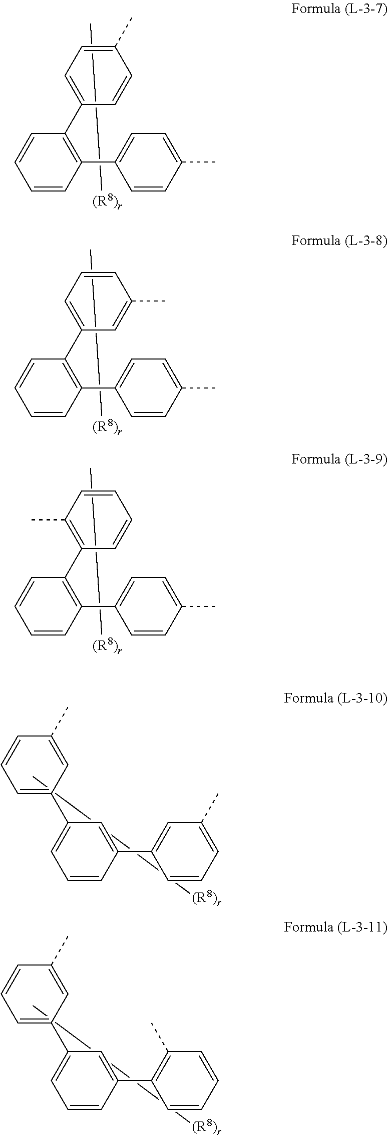

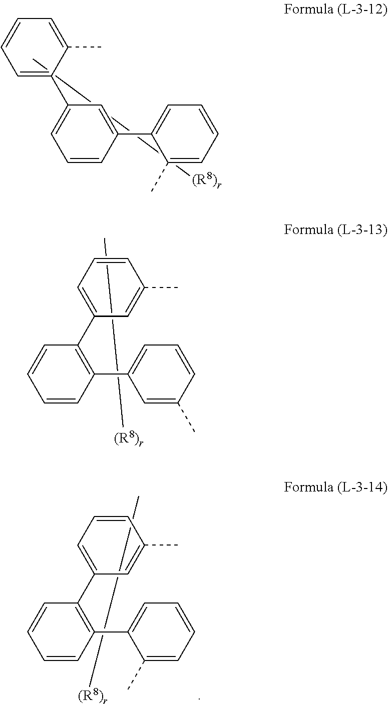

25. Compound according to claim 20, characterized in that L is selected from the following formulae:

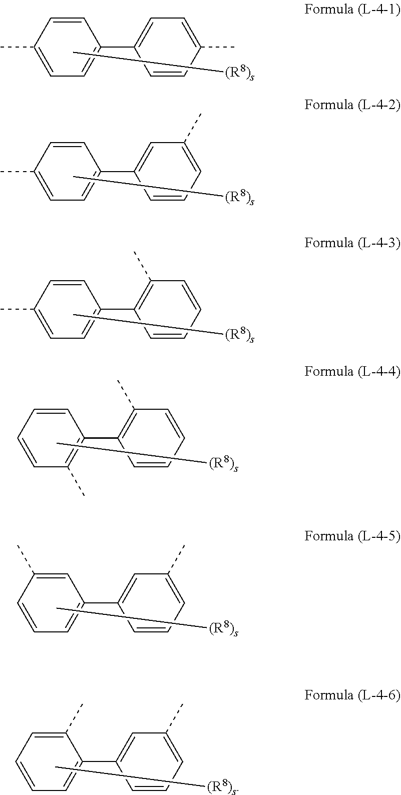

26. Compound according to claim 20, characterized in that the compound is selected from the following formulae:

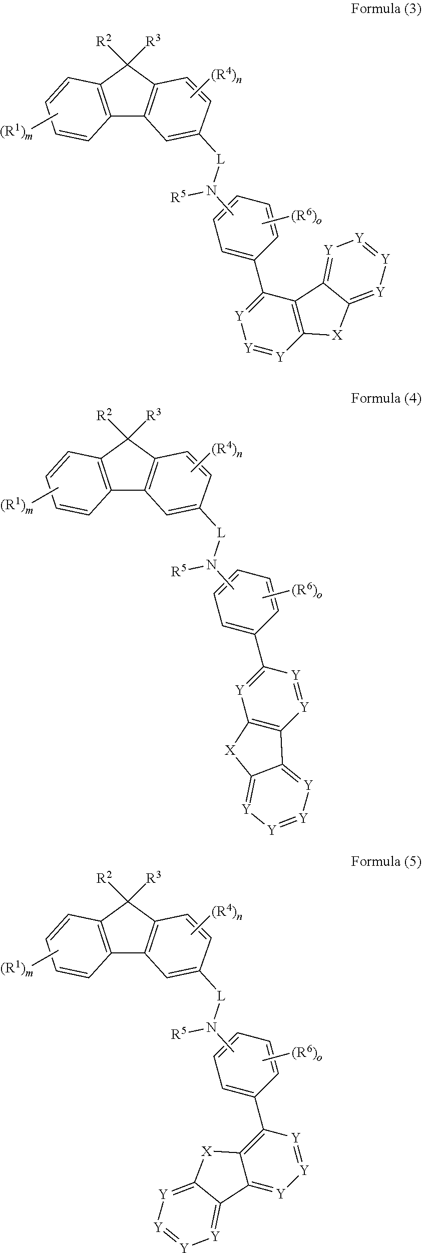

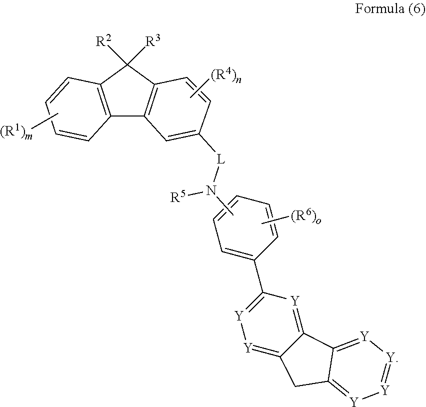

27. Compound according to claim 20, characterized in that it is a monoamine compound.

28. Compound according to claim 20, characterized in that R² and R³ are the same or different at each instance and are selected from straight-chain alkyl groups having 1 to 10 carbon atoms, branched or cyclic alkyl groups having 3 to 10 carbon atoms or aromatic ring systems having 6 to 18 aromatic atoms; where the two R² and R³ radicals may be joined to one another and may form a ring; where the alkyl groups mentioned and the aromatic ring systems mentioned may each be substituted by R¹¹ radicals; if the two R² and R³ radicals form a ring, the result is a spiro compound, preferably a spirobifluorene.

29. Compound according to claim 20, characterized in that the R⁵ radicals are selected from the following formulae: 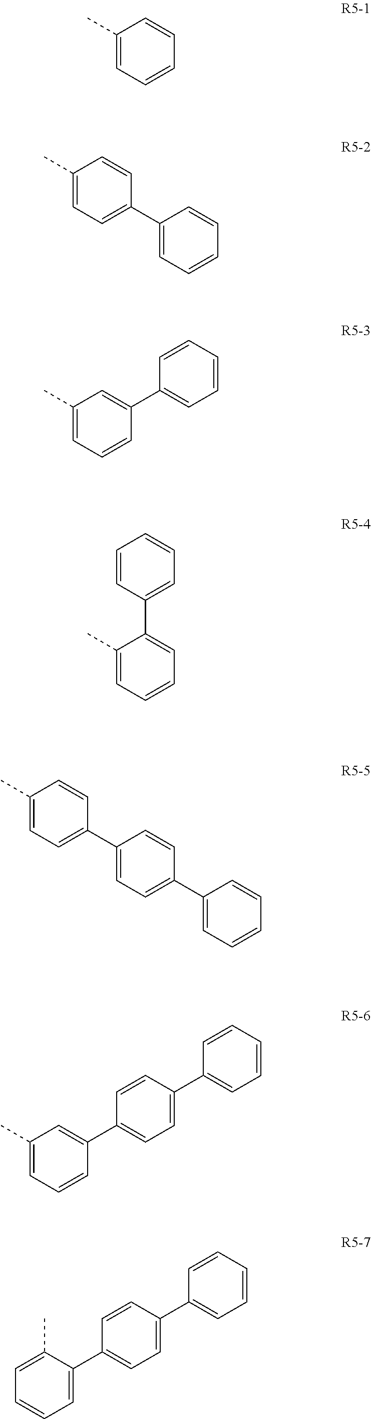 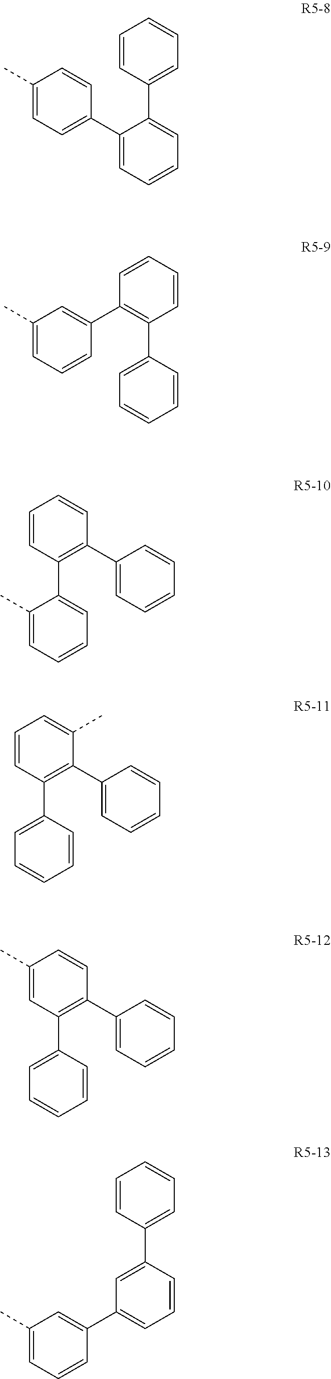 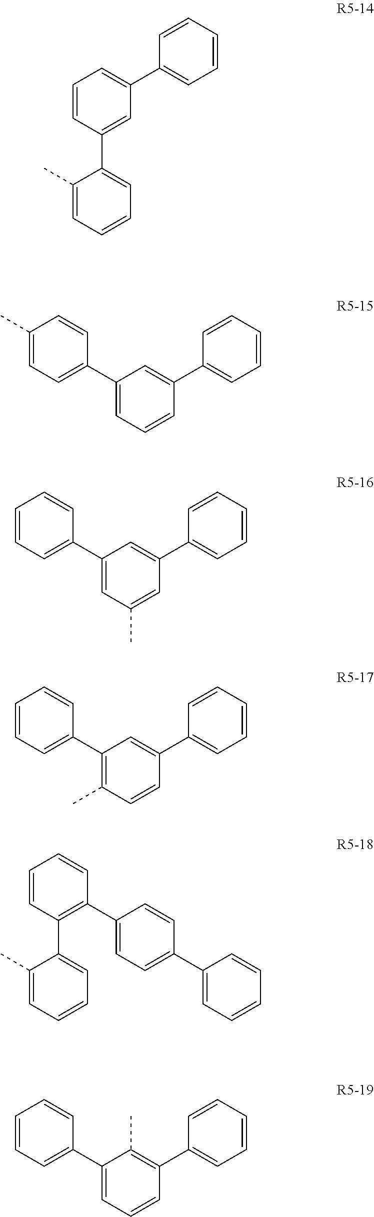 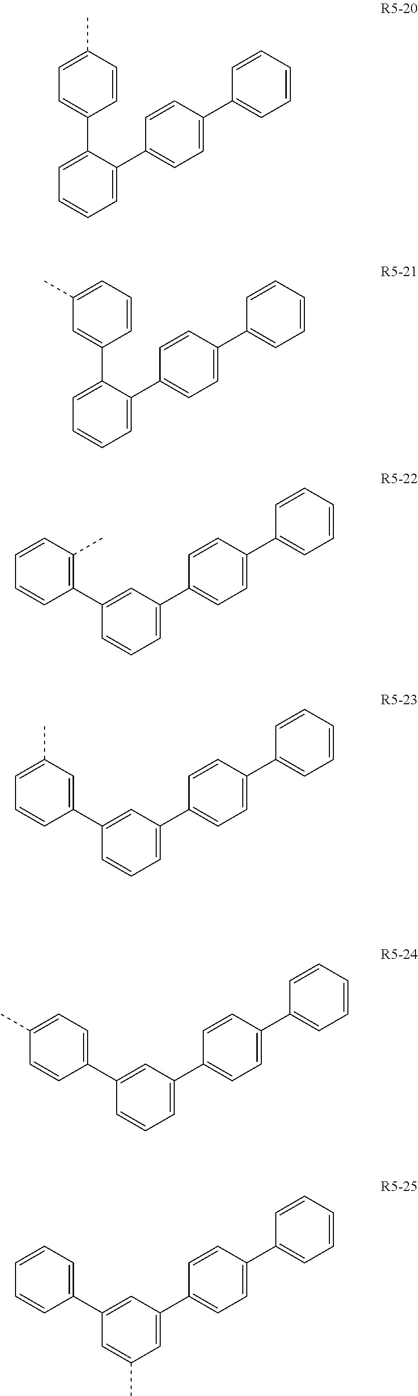 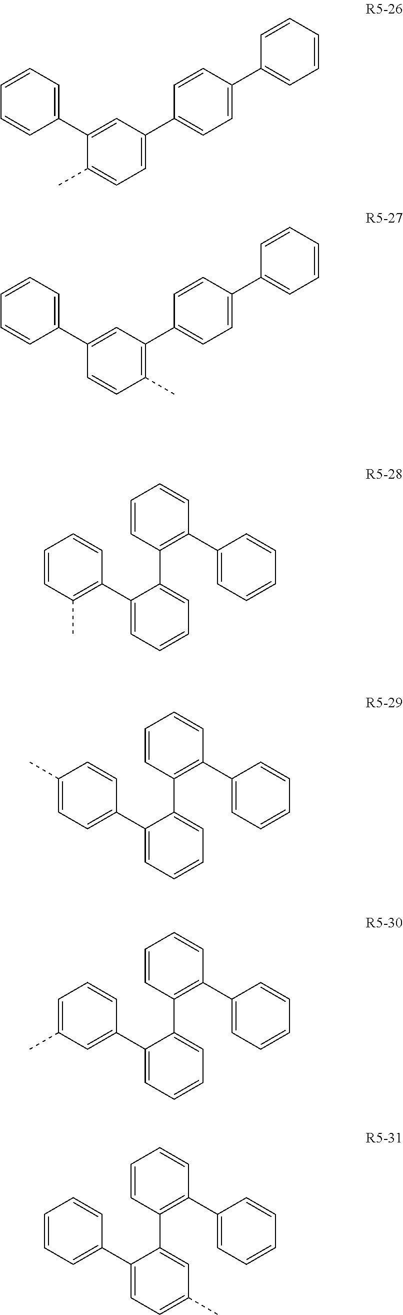 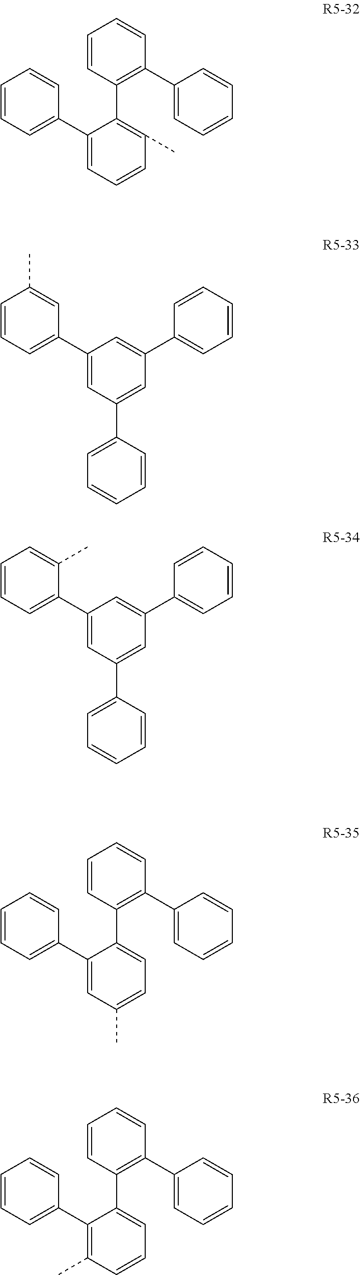 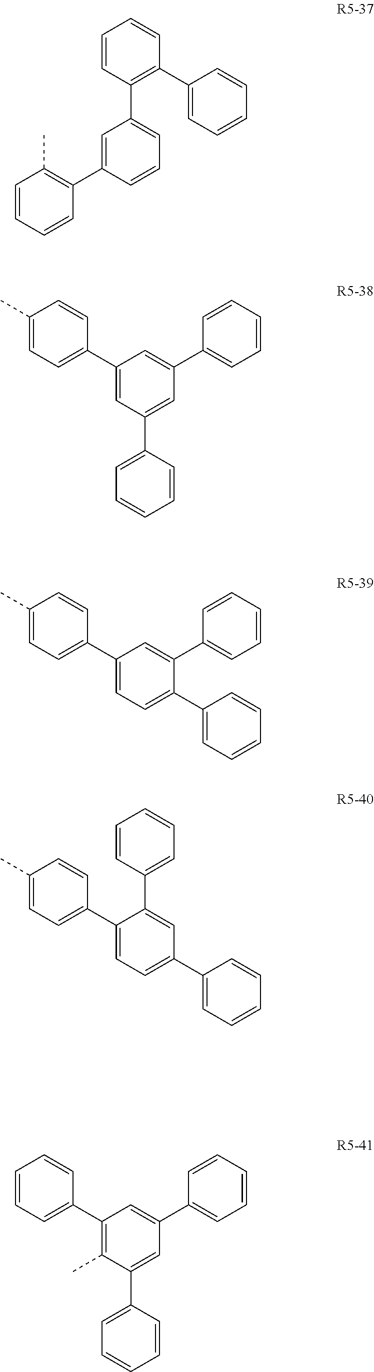 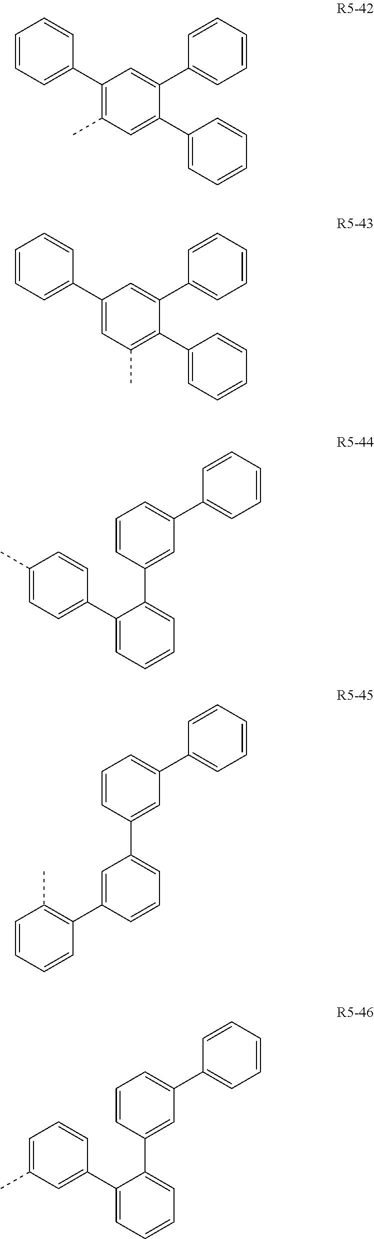 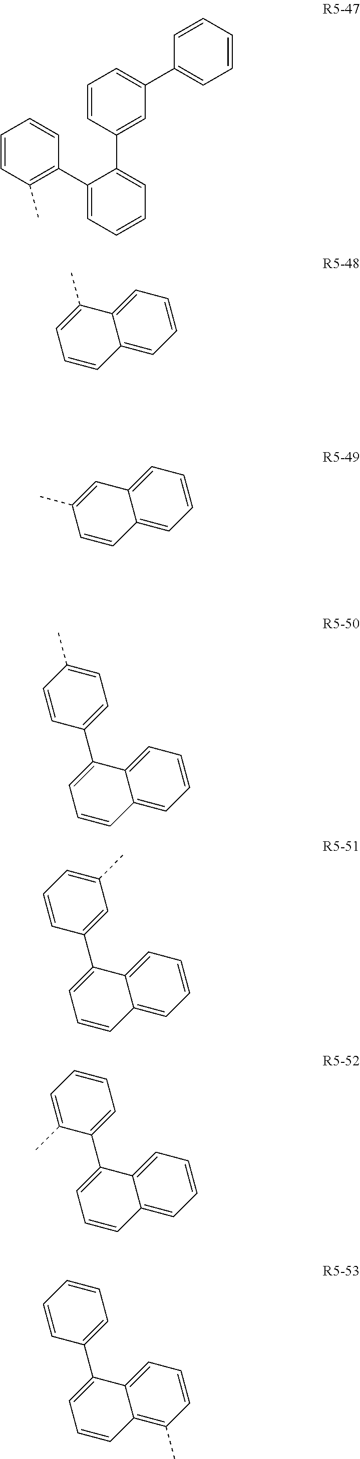 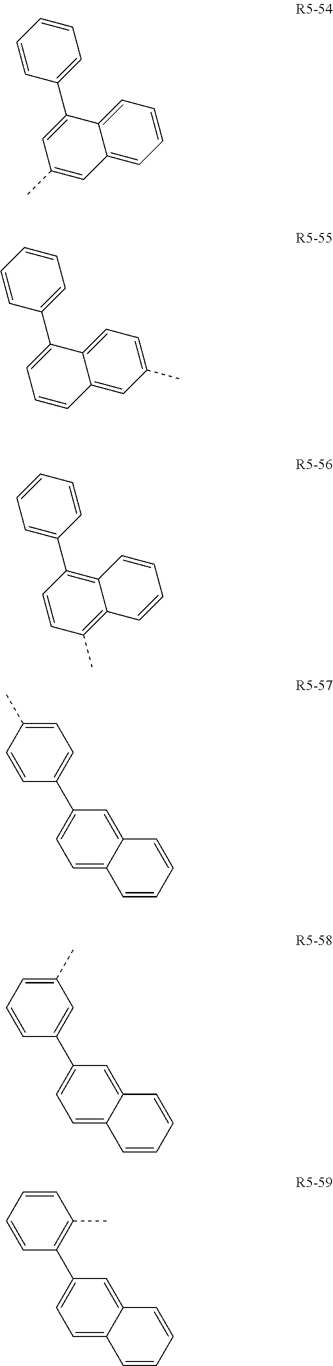 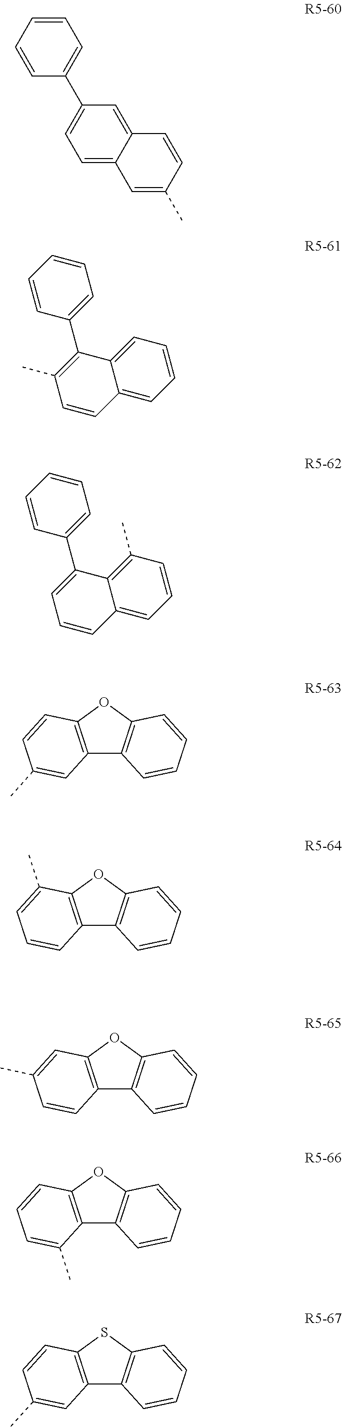 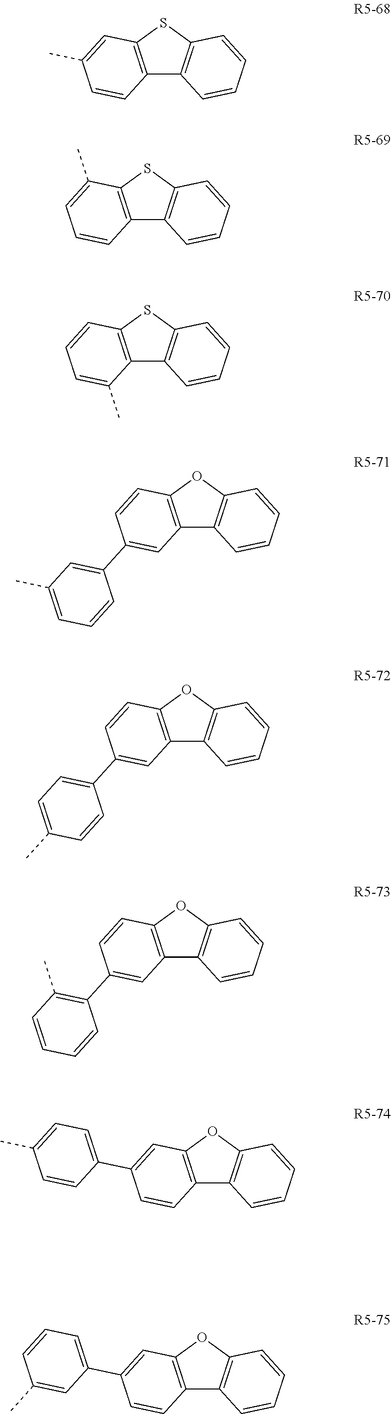 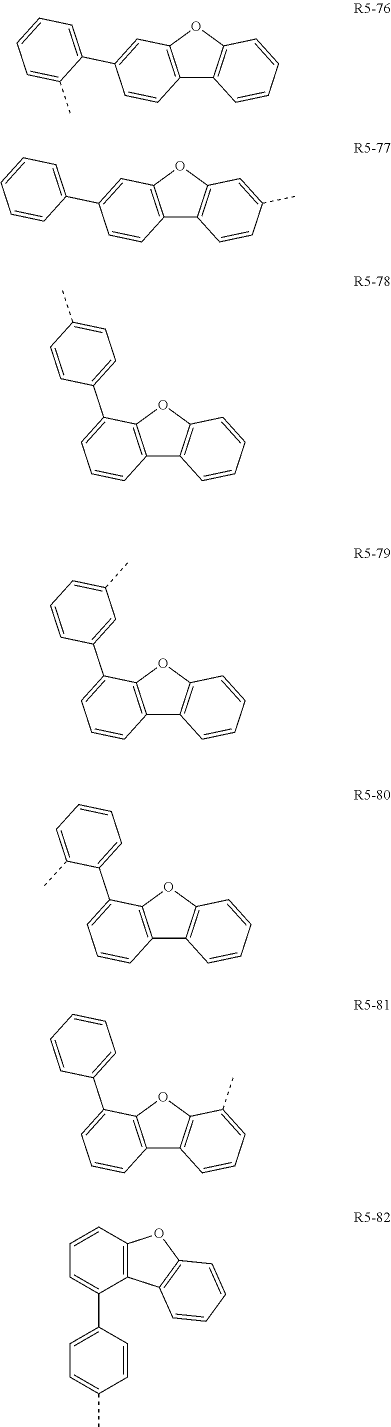 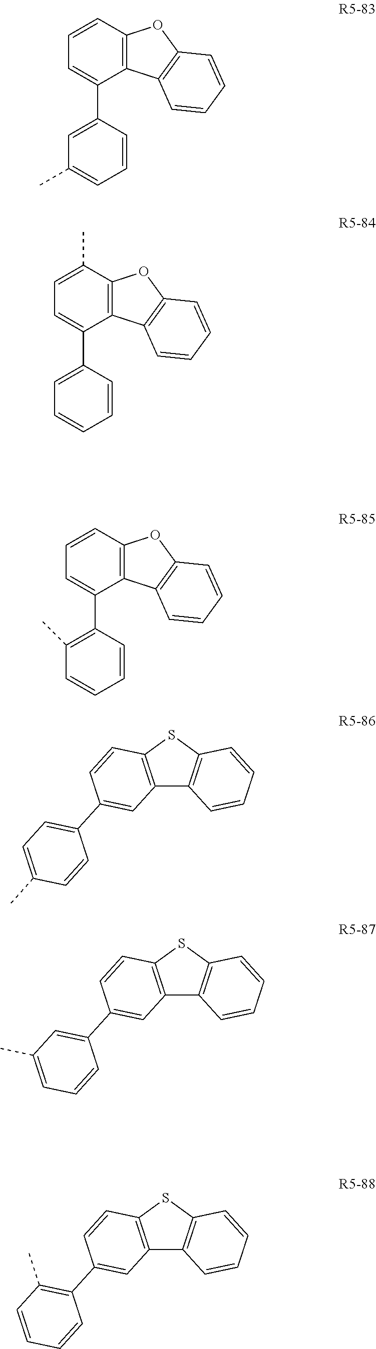 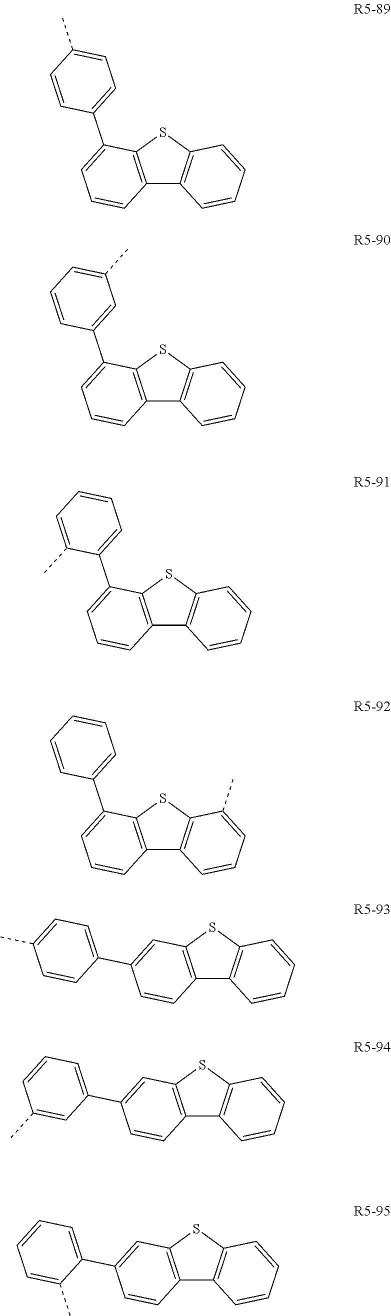 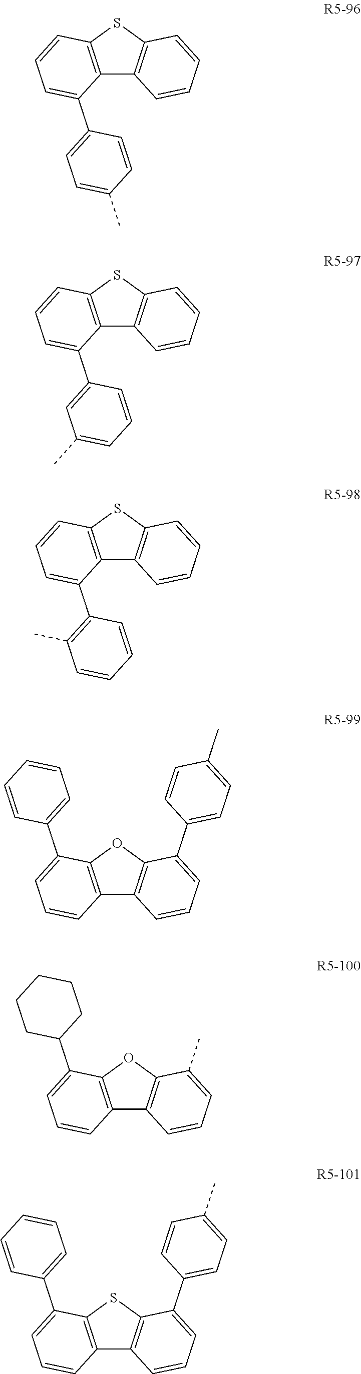 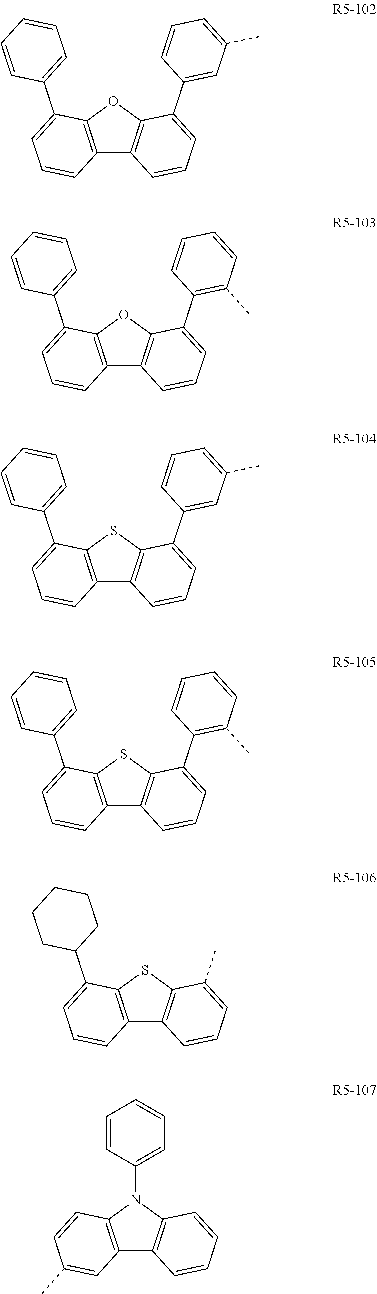 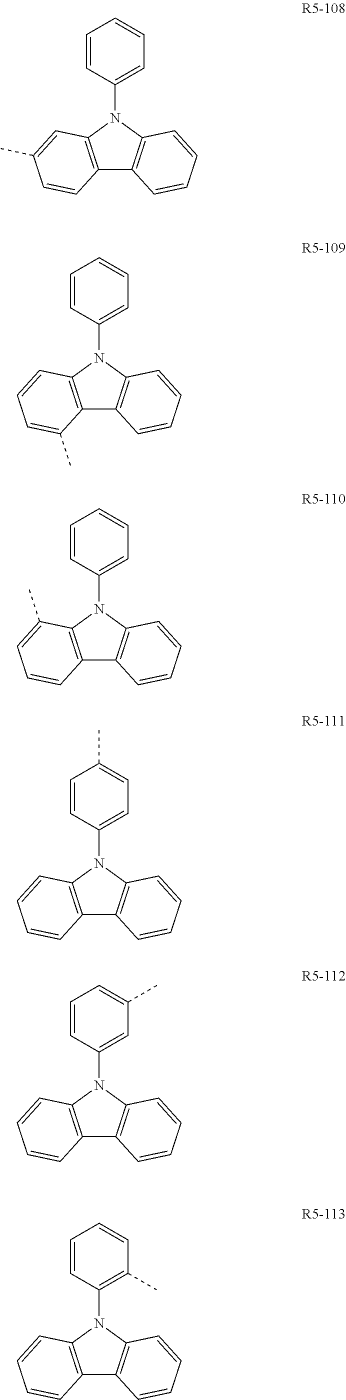 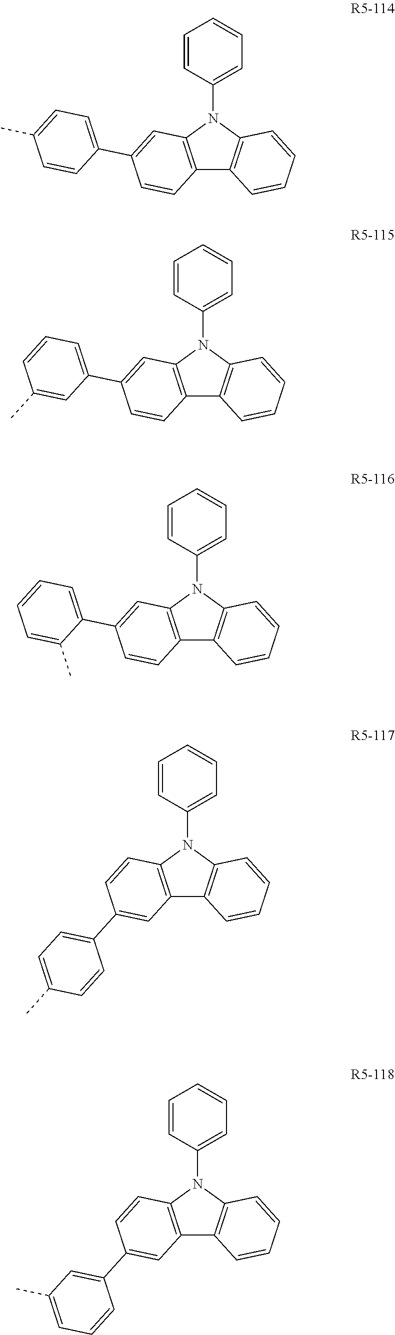 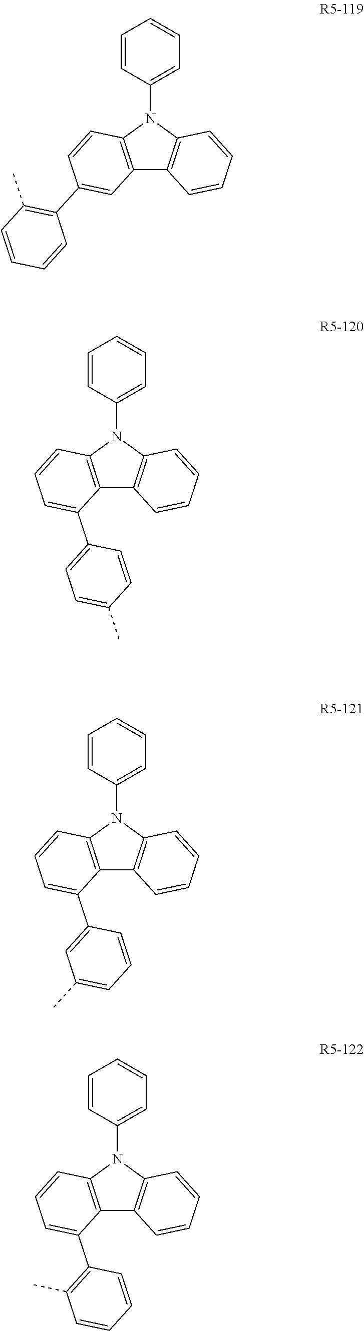 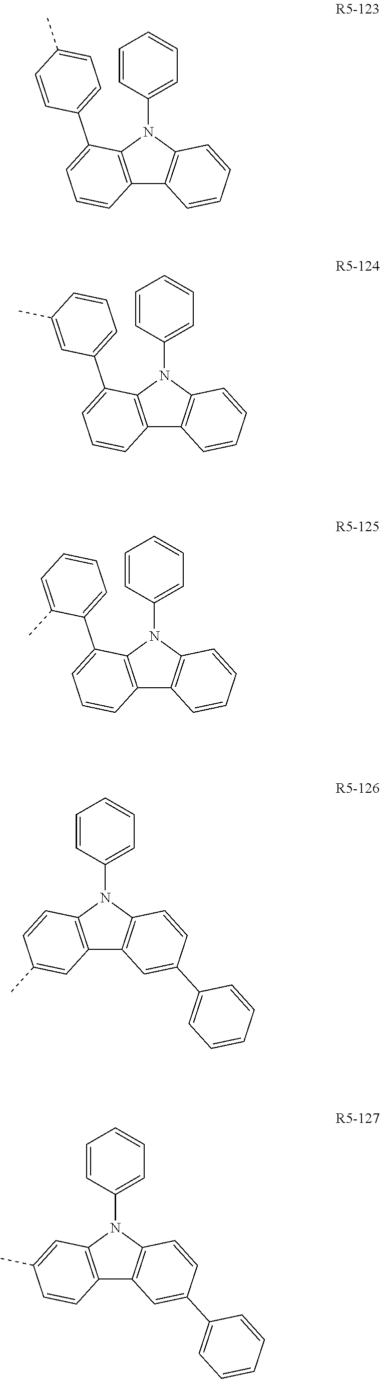 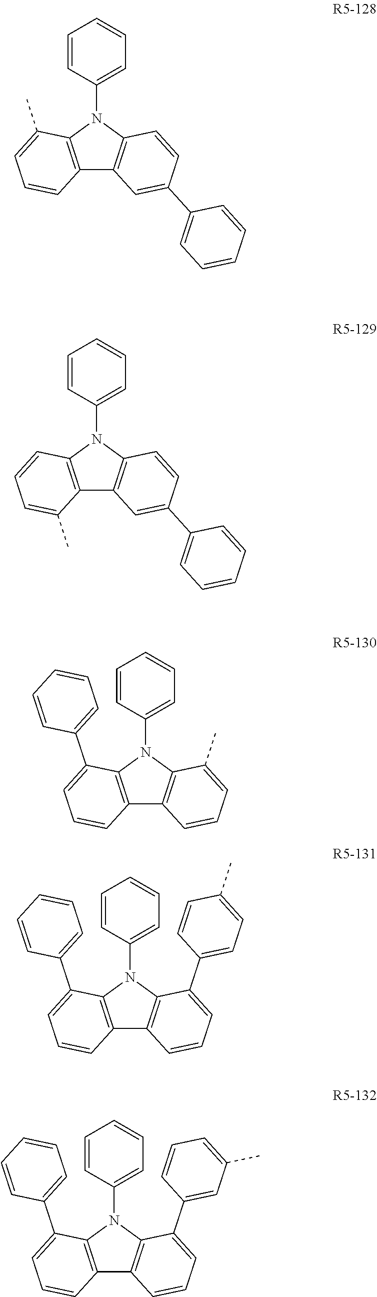 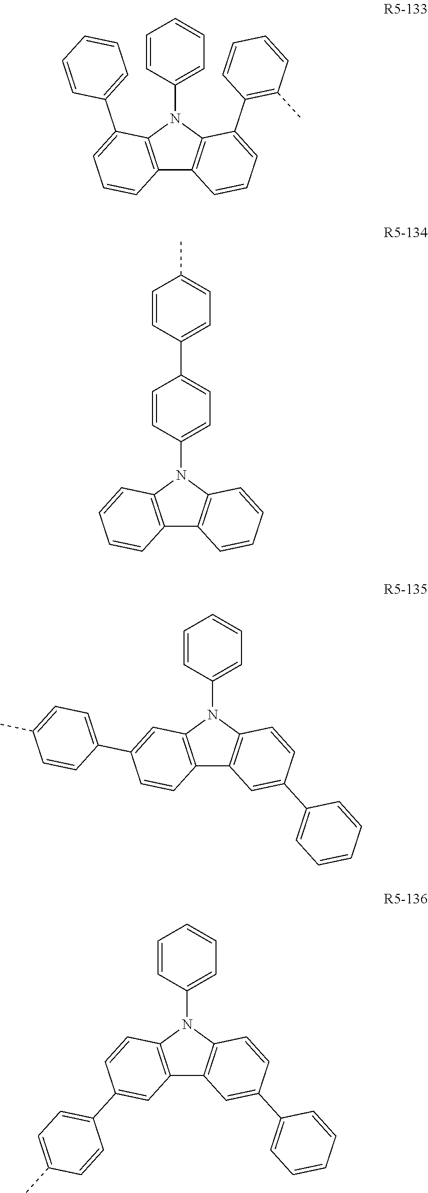 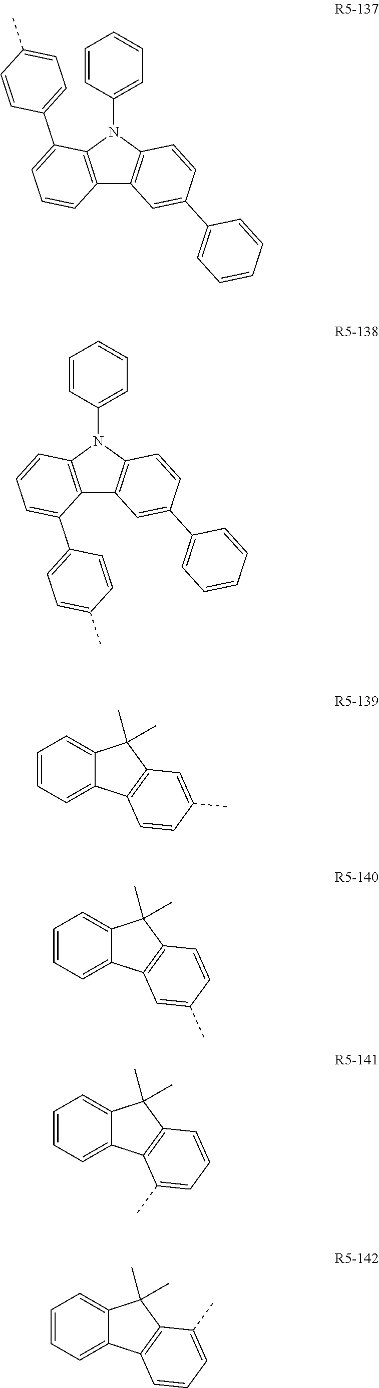 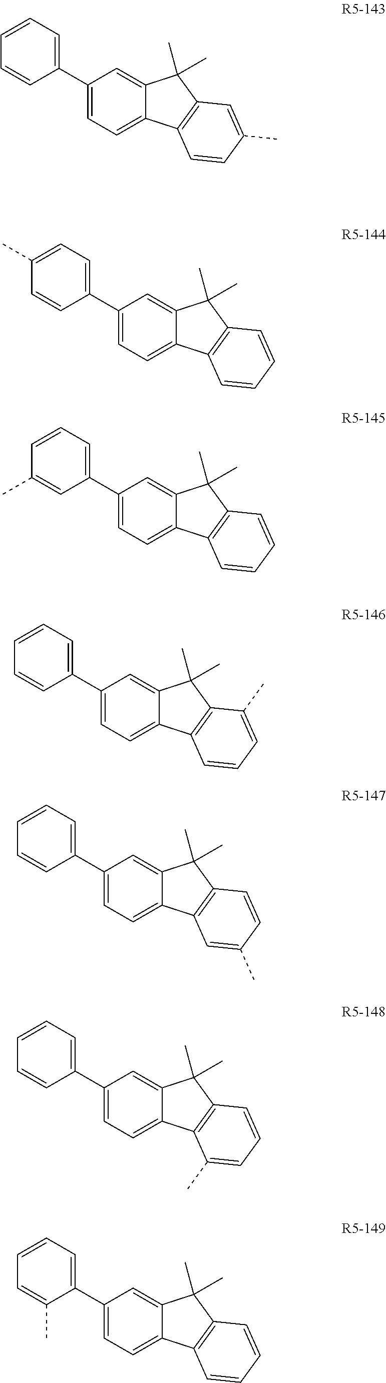 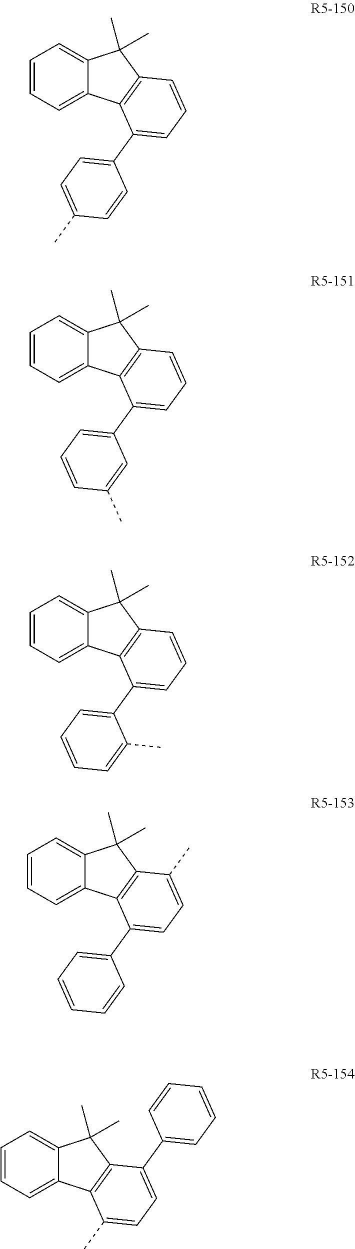 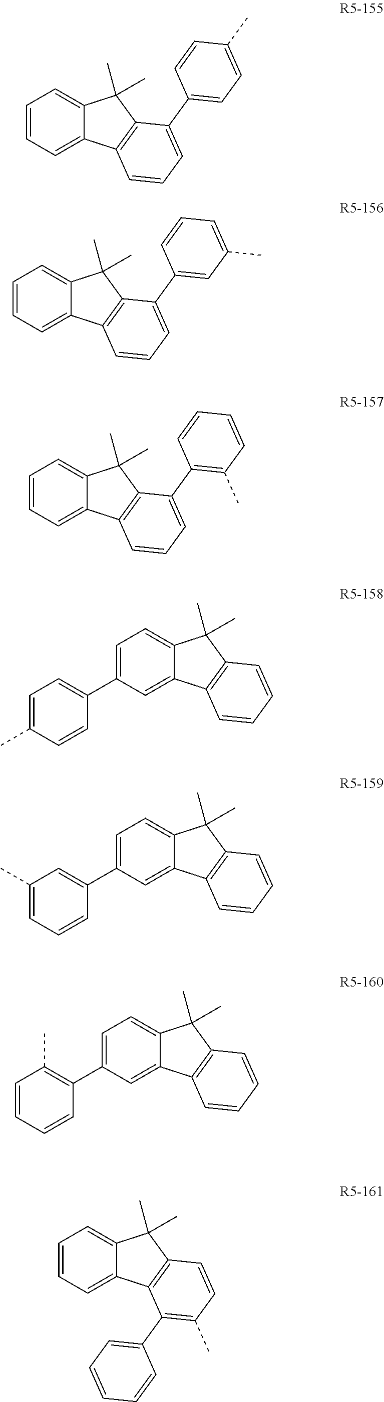 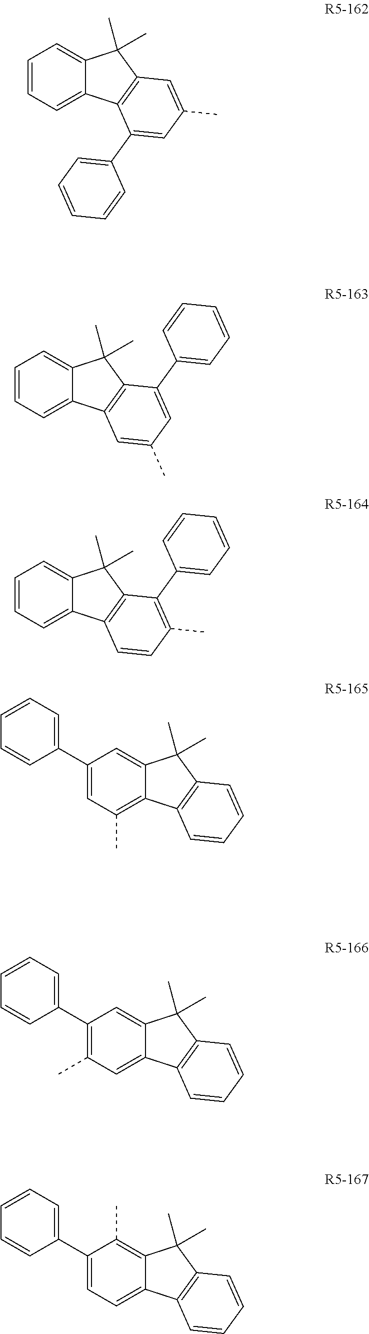 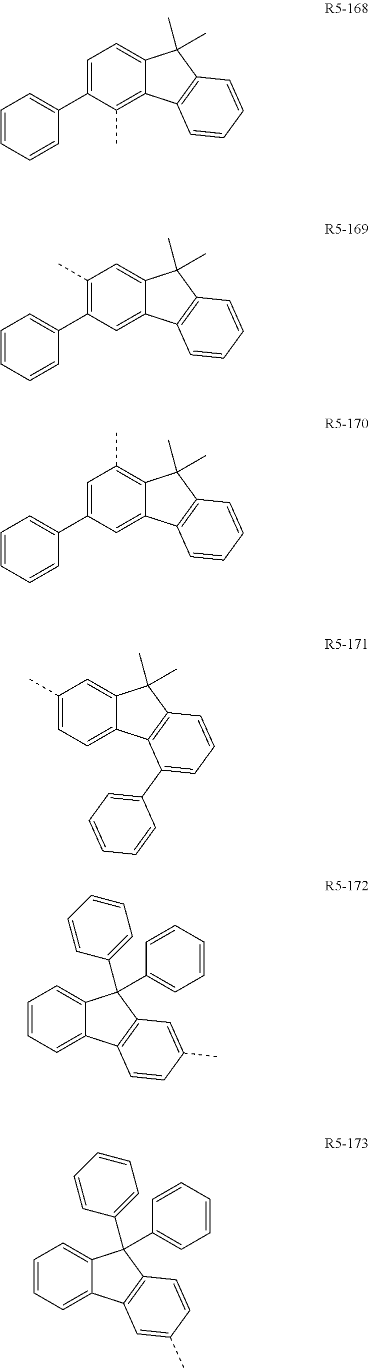 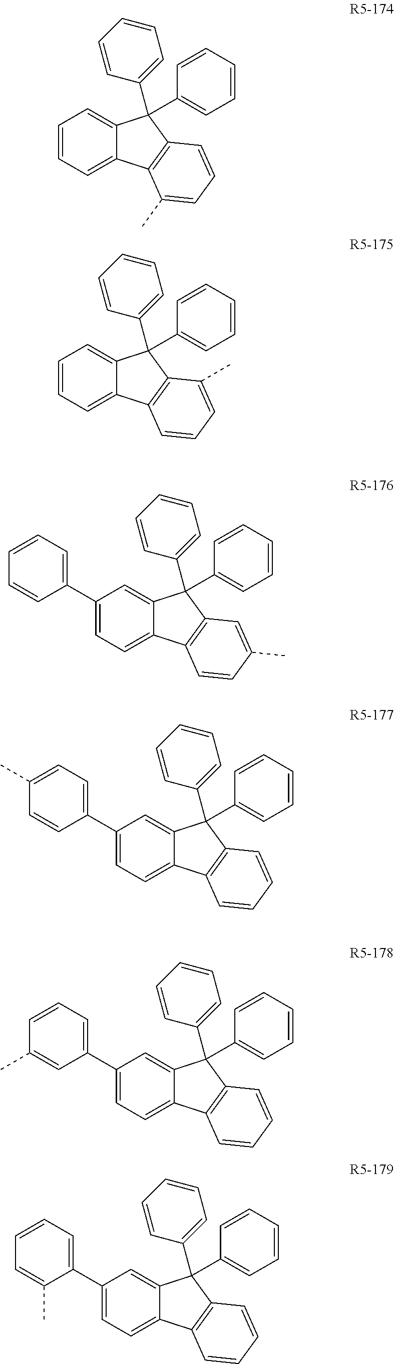 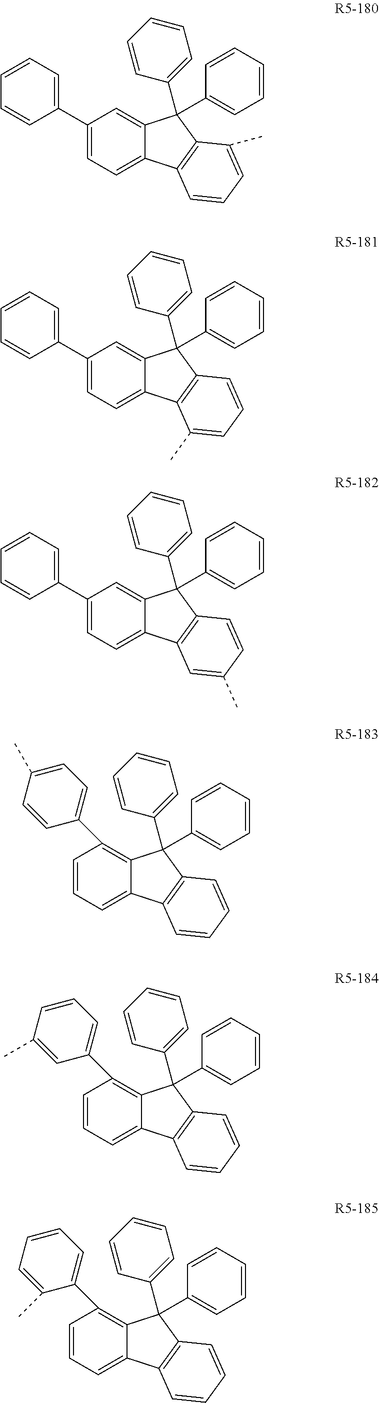 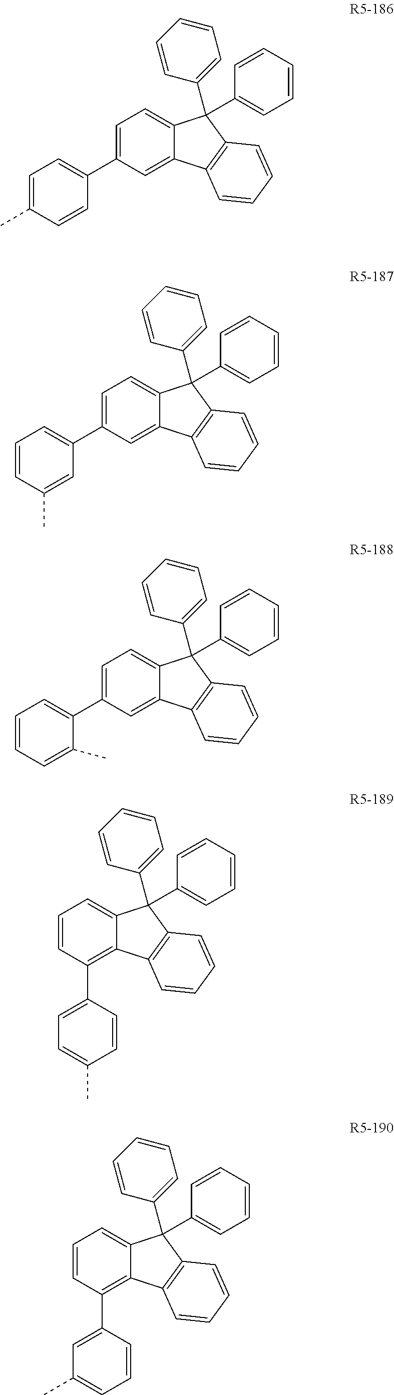 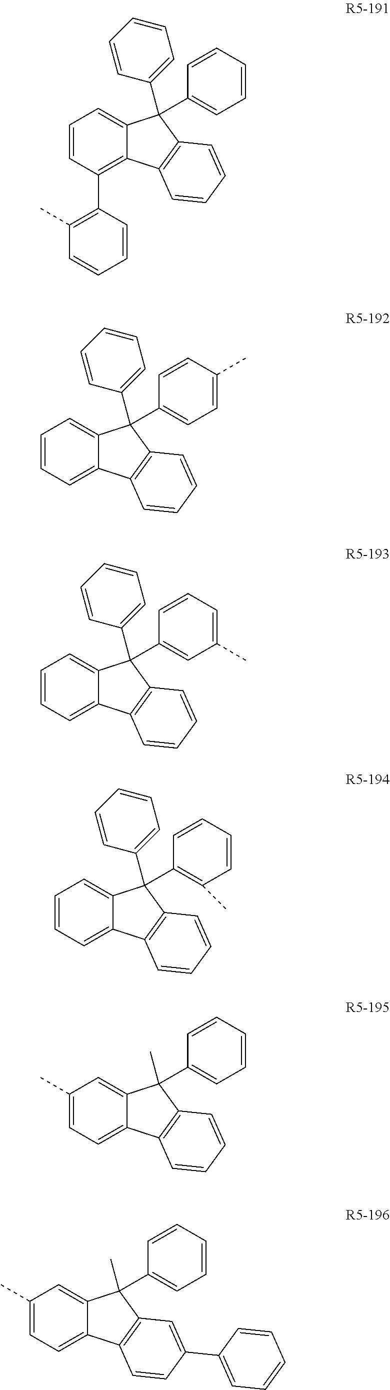 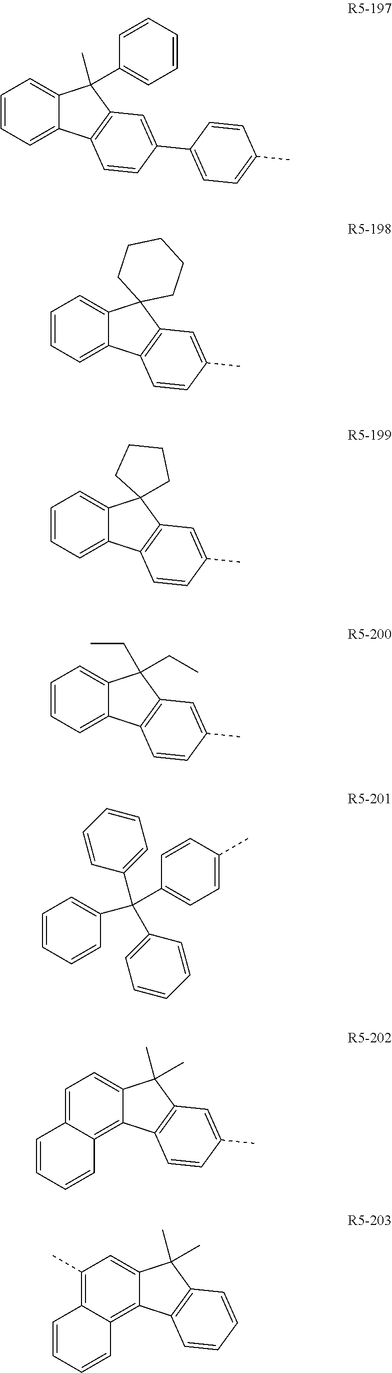 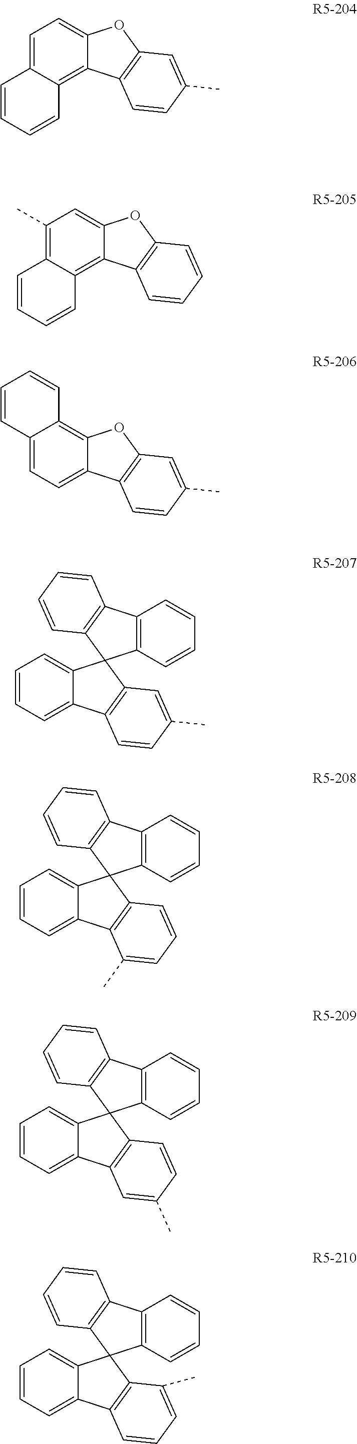 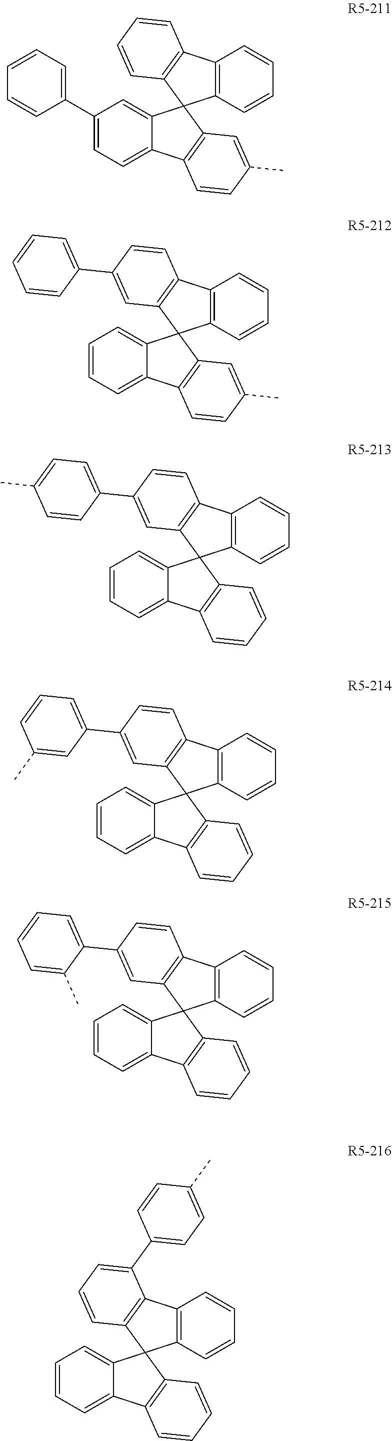 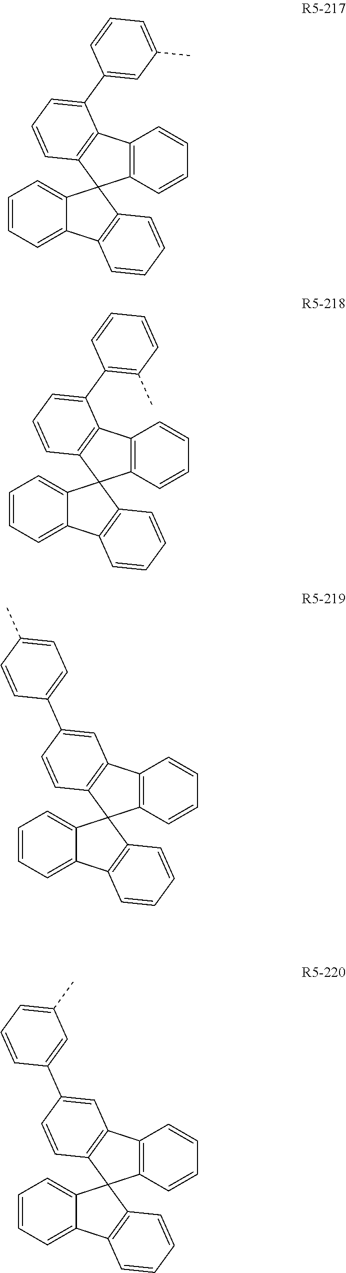 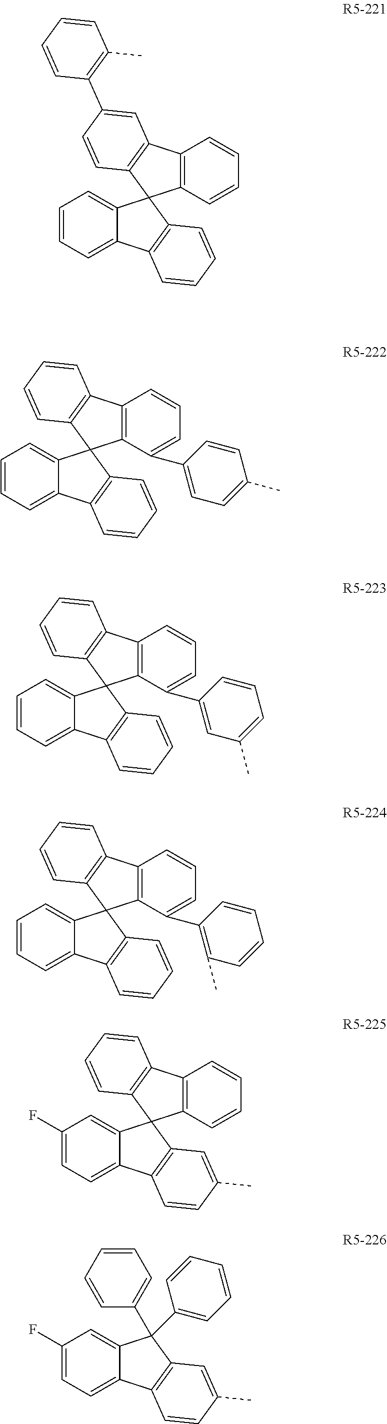 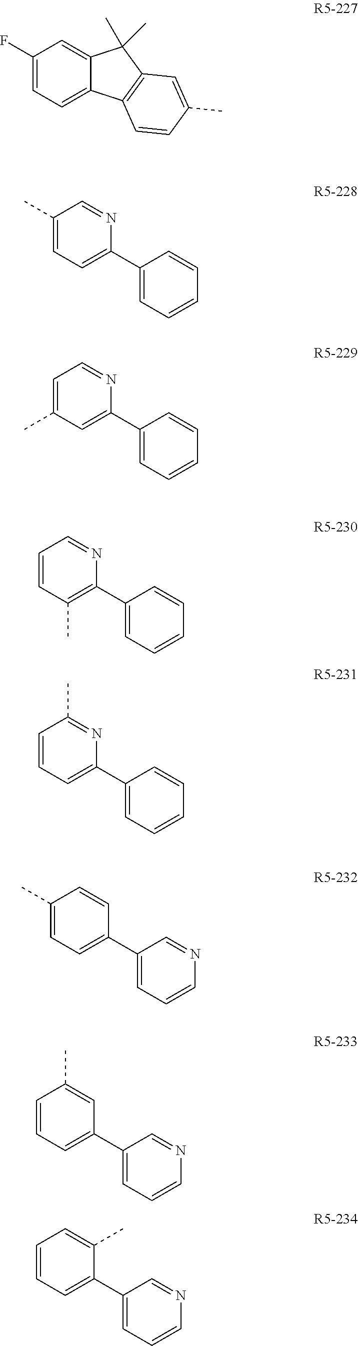 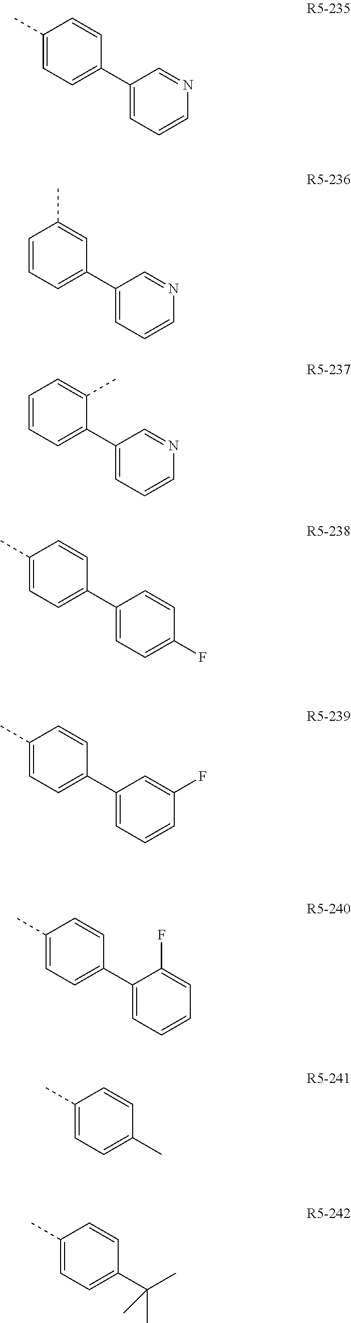 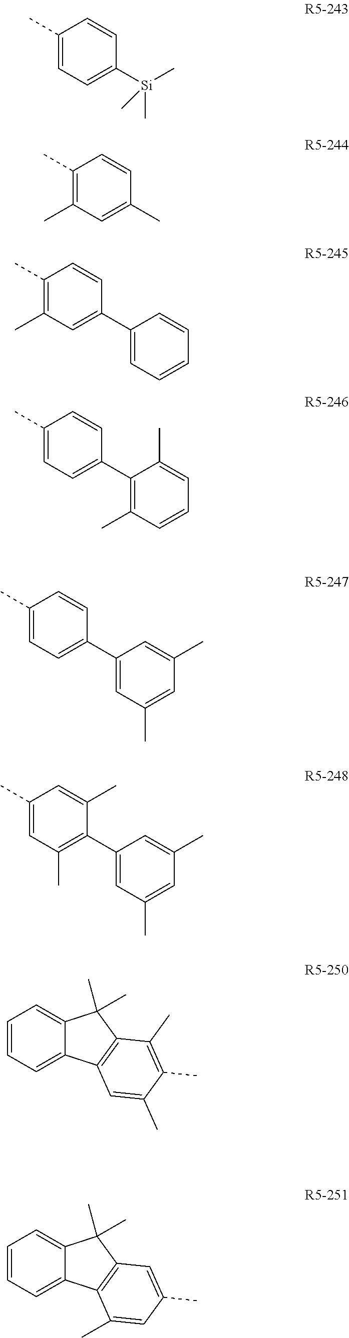 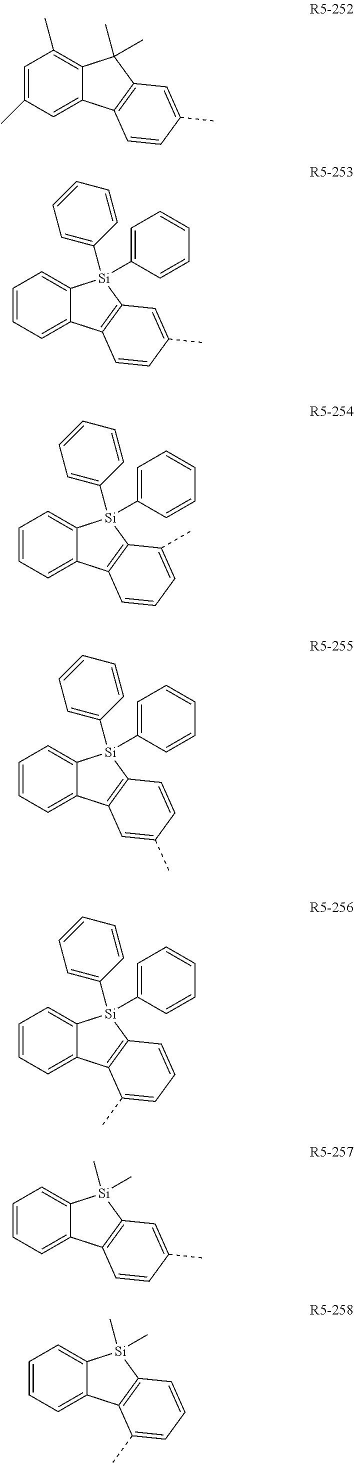 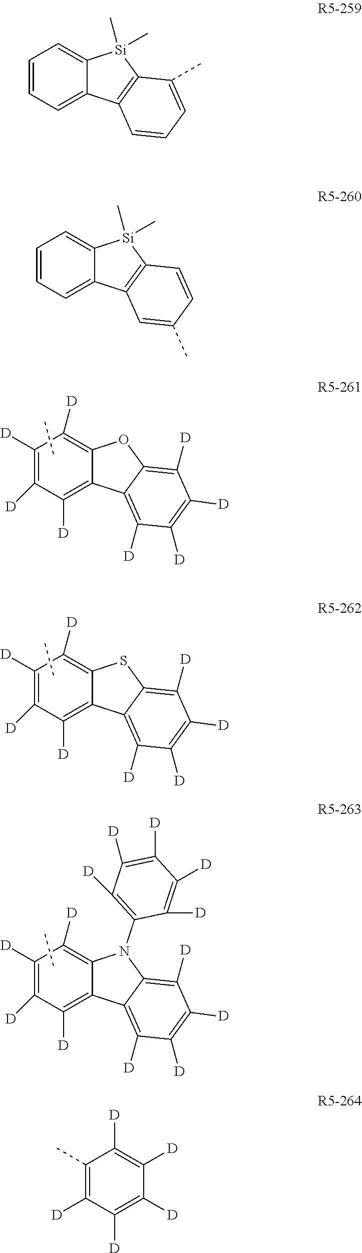 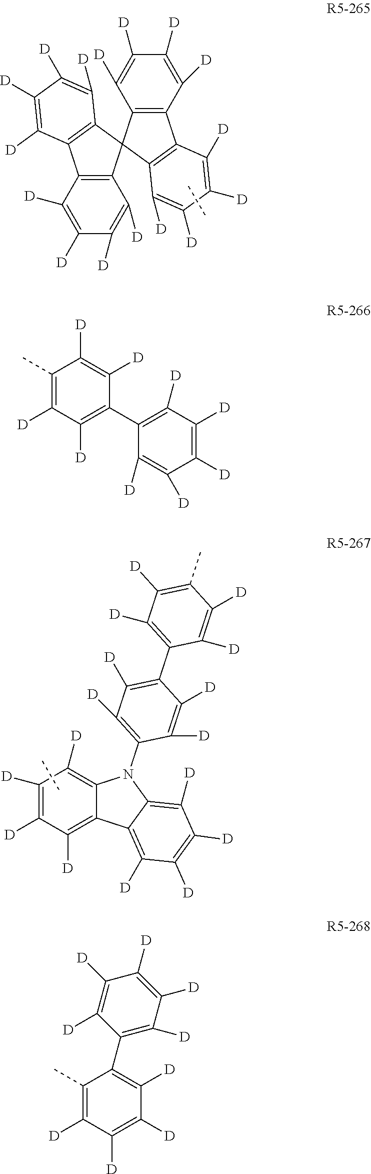 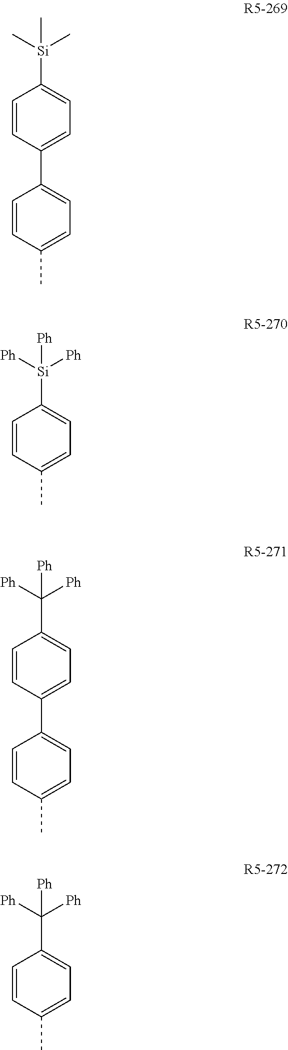
where the R⁵ radicals mentioned may be substituted by R¹¹ radicals at the positions shown as unsubstituted, and where the dotted bond is the bond to the amine nitrogen atom.

30. Compound according to claim 20, characterized in that m, n and o are 0.

31. Process for preparing a compound according to claim 20 with the aid of Suzuki coupling or the Buchwald reaction.

32. Oligomer, polymer or dendrimer containing one or more compounds according to claim 20, wherein the bond(s) to the polymer, oligomer or dendrimer may be localized at any desired positions substituted by R¹, R², R³, R⁴, R⁵, R⁶, R⁷, R⁸, R¹¹ or R¹² in formula (1).

33. Composition comprising one or more compounds of the formula (1) and at least one further material selected from the group consisting of the hole transport materials, hole injection materials, p-dopants, electron blocker materials, matrix materials, emitters and electron transport materials.

34. Formulation comprising at least one compound according to claim 20, and at least one solvent.

35. Electronic device comprising at least one compound according to claim 20.

36. Electronic device according to claim 35, characterized in that it is an organic electroluminescent device and comprises anode, cathode and at least one emitting layer, and in that the compound is present in an electron-blocking layer or in a hole-transporting layer or in an emitting layer of the device.

37. Organic electroluminescent device according to claim 36, characterized in that the compound is present in a hole-transporting layer which is a hole transport layer or an electron blocker layer.

38. Use of a compound according to claim 20 in an electronic device.
